(19) United States
(12) Patent Application Publication
(10) Pub. No.: US 2025/0009904 A1
(43) Pub. Date: Jan. 9, 2025

(54) RNA-Editing Compositions and Methods of Use

(71) Applicant: Shape Therapeutics Inc., Seattle, WA (US)

(72) Inventors: Yiannis SAVVA, Seattle, WA (US); Jason Thaddeus DEAN, Seattle, WA (US); Brian John BOOTH, West Roxbury, MA (US); Richard Thomas SULLIVAN, Seattle, WA (US); Adrian Wrangham BRIGGS, Seattle, WA (US); Lina Rajili BAGEPALLI, Cambridge, MA (US); Yazmin Ines ROVIRA GONZALEZ, Seattle, WA (US); Lan GUO, Seattle, WA (US)

(21) Appl. No.: 18/641,539

(22) Filed: Apr. 22, 2024

Related U.S. Application Data
(63) Continuation of application No. PCT/US2022/078740, filed on Oct. 26, 2022.
(60) Provisional application No. 63/271,889, filed on Oct. 26, 2021.
(60) Provisional application No. 63/277,707, filed on Nov. 10, 2021.
(60) Provisional application No. 63/284,737, filed on Dec. 1, 2021.
(60) Provisional application No. 63/296,955, filed on Jan. 6, 2022.
(60) Provisional application No. 63/303,659, filed on Jan. 27, 2022.
(60) Provisional application No. 63/306,809, filed on Feb. 4, 2022.
(60) Provisional application No. 63/327,380, filed on Apr. 5, 2022.
(60) Provisional application No. 63/345,069, filed on May 24, 2022.

(51) Int. Cl. A61K 48/00; C12N 9/78; C12N 15/11

(52) U.S. Cl. CPC A61K 48/005; C12N 9/78; C12N 15/111; C12N 2310/20; C12N 2310/531; C12N 2310/533; C12Y 305/04004

(57) ABSTRACT

Provided herein are engineered guides configured, upon hybridization to target RNA molecules, to form double stranded RNA substrates comprising (i) a region comprising at least one structural feature; and (ii) a first internal loop and a second internal loop, wherein the double stranded RNA substrates recruit RNA editing entities and facilitate chemical modifications of base nucleotides in the target RNA molecules. Also provided herein are compositions, vectors, and cells comprising the engineered guides disclosed herein. Also provided herein are methods of introducing the engineered guides described herein into cells and methods of treating a disease or condition in a subject in need thereof comprising administering to the subject the engineered guides, polynucleotides encoding the engineered guides, delivery vehicles comprising such engineered guides or such polynucleotides, or pharmaceutical compositions comprising any one of these as described herein.

### CROSS REFERENCE

This application is a continuation of PCT/US2022/078740, filed Oct. 26, 2022, which claims priority under 35 U.S.C. § 119 from Provisional Application Ser. No. 63/271,889, filed Oct. 26, 2021, Provisional Application Ser. No. 63/277,707, filed Nov. 10, 2021, Provisional Application Ser. No. 63/284,737, filed Dec. 1, 2021, Provisional Application Ser. No. 63/296,955, filed Jan. 6, 2022, Provisional Application Ser. No. 63/303,659, filed Jan. 27, 2022, Provisional Application Ser. No. 63/306,809, filed Feb. 4, 2022, Provisional Application Ser. No. 63/327,380, filed Apr. 5, 2022, and Provisional Application Ser. No. 63/345,069, filed May 24, 2022, the disclosures of which are incorporated herein by reference in their entirety.

### SEQUENCE LISTING

The instant application contains a Sequence Listing which has been submitted electronically in ST.26 (xml) format and is hereby incorporated by reference in its entirety. Said ST.26 (xml) copy, created on Apr. 15, 2024, is named 199235_743301_xml and is 1,576,960 bytes in size.

### SUMMARY

Disclosed herein is an engineered guide RNA or a polynucleotide sequence encoding the engineered guide RNA, wherein upon hybridization of the engineered guide RNA to a sequence of a target RNA, the engineered guide RNA and the sequence of the target RNA form a guide-target RNA scaffold, wherein the guide-target RNA scaffold comprises: (i) a region that comprises at least one structural feature selected from the group consisting of: a bulge, a wobble base pair, an internal loop, a mismatch, a hairpin, and any combination thereof, wherein, upon contacting the guide-target RNA scaffold with an RNA editing entity, the RNA editing entity edits an on-target adenosine in the target RNA within the guide-target RNA scaffold; and (ii) a first internal loop and a second internal loop that flank opposing ends of the region of the guide-target RNA scaffold of (i), wherein the first internal loop is 5′ of the region that comprises the at least one structural feature and the second internal loop is a 3′ of the region that comprises the at least one structural feature, and wherein the first internal loop and the second internal loop facilitate an increase in the amount of the editing of the on-target adenosine in the target RNA, relative to an otherwise comparable engineered guide RNA lacking the first internal loop and the second internal loop. In some embodiments, the first internal loop and the second internal loop facilitate a decrease in the amount of off-target adenosine editing in the target RNA, relative to an otherwise comparable engineered guide RNA lacking the first internal loop and the second internal loop. In some embodiments, the first internal loop is a symmetric internal loop and the second internal loop is a symmetric internal loop. In some embodiments, the first internal loop and the second internal loop are symmetric internal loops that independently are 5/5, 6/6, 7/7, 8/8, 9/9, 10/10, 11/11, 12/12, 13/13, 14/14, 15/15, 16/16, 17/17, 18/18, 19/19, or 20/20 symmetric internal loops, wherein the first number is the number of nucleotides contributed to the symmetric internal loop from the engineered guide RNA side of the guide-target RNA scaffold and the second number is the number of nucleotides contributed to the symmetric internal loop from the target RNA side of the guide-target RNA scaffold. In some embodiments, the first internal loop is an asymmetric internal loop and the second internal loop is an asymmetric internal loop. In some embodiments, the first internal loop and the second internal loop are asymmetric internal loops that independently are 5/6, 5/7, 5/8, 5/9, 5/10, 5/11, 5/12, 5/13, 5/14, 5/15, 5/16, 5/17, 5/18, 5/19, 5/20, 6/5, 6/7, 6/8, 6/9, 6/10, 6/11, 6/12, 6/13, 6/14, 6/15, 6/16, 6/17, 6/18, 6/19, 6/20, 7/5, 7/6, 7/8, 7/9, 7/10, 7/11, 7/12, 7/13, 7/14, 7/15, 7/16, 7/17, 7/18, 7/19, 7/20, 8/5, 8/6, 8/7, 8/9, 8/10, 8/11, 8/12, 8/13, 8/14, 8/15, 8/16, 8/17, 8/18, 8/19, 8/20, 9/5, 9/6, 9/7, 9/8, 9/10, 9/11, 9/12, 9/13, 9/14, 9/15, 9/16, 9/17, 9/18, 9/19, 9/20, 10/5, 10/6, 10/7, 10/8, 10/9, 10/11, 10/12, 10/13, 10/14, 10/15, 10/16, 10/17, 10/18, 10/19, 10/20, 11/5, 11/6, 11/7, 11/8, 11/9, 11/10, 11/12, 11/13, 11/14, 11/15, 11/16, 11/17, 11/18, 11/19, 11/20, 12/5, 12/6, 12/7, 12/8, 12/9, 12/10, 12/11, 12/13, 12/14, 12/15, 12/16, 12/17, 12/18, 12/19, 12/20, 13/5, 13/6, 13/7, 13/8, 13/9, 13/10, 13/11, 13/12, 13/14, 13/15, 13/16, 13/17, 13/18, 13/19, 13/20, 14/5, 14/6, 14/7, 14/8, 14/9, 14/10, 14/11, 14/12, 14/13, 14/15, 14/16, 14/17, 14/18, 14/19, 14/20, 15/5, 15/6, 15/7, 15/8, 15/9, 15/10, 15/11, 15/12, 15/13, 15/14, 15/16, 15/17, 15/18, 15/19, 15/20, 16/5, 16/6, 16/7, 16/8, 16/9, 16/10, 16/11, 16/12, 16/13, 16/14, 16/15, 16/17, 16/18, 16/19, 16/20, 17/5, 17/6, 17/7, 17/8, 17/9, 17/10, 17/11, 17/12, 17/13, 17/14, 17/15, 17/16, 17/18, 17/19, 17/20, 18/5, 18/6, 18/7, 18/8, 18/9, 18/10, 18/11, 18/12, 18/13, 18/14, 18/15, 18/16, 18/17, 18/19, 18/20, 19/5, 19/6, 19/7, 19/8, 19/9, 19/10, 19/11, 19/12, 19/13, 19/14, 19/15, 19/16, 19/17, 19/18, 19/20, 20/5, 20/6, 20/7, 20/8, 20/9, 20/10, 20/11, 20/12, 20/13, 20/14, 20/15, 20/16, 20/17, 20/18, or 20/19 asymmetric internal loops, wherein the first number is the number of nucleotides contributed to the asymmetric internal loop from the engineered guide RNA side of the guide-target RNA scaffold and the second number is the number of nucleotides contributed to the asymmetric internal loop from the target RNA side of the guide-target RNA scaffold. In some embodiments, the first internal loop is a symmetric internal loop and the second internal loop is an asymmetric internal loop. In some embodiments, the first internal loop is a symmetric internal loop that is a 5/5, 6/6, 7/7, 8/8, 9/9, 10/10, 11/11, 12/12, 13/13, 14/14, 15/15, 16/16, 17/17, 18/18, 19/19, or 20/20 symmetric internal loop; and wherein the second internal loop is an asymmetric internal loop that is a 5/6, 5/7, 5/8, 5/9, 5/10, 5/11, 5/12, 5/13, 5/14, 5/15, 5/16, 5/17, 5/18, 5/19, 5/20, 6/5, 6/7, 6/8, 6/9, 6/10, 6/11, 6/12, 6/13, 6/14, 6/15, 6/16, 6/17, 6/18, 6/19, 6/20, 7/5, 7/6, 7/8, 7/9, 7/10, 7/11, 7/12, 7/13, 7/14, 7/15, 7/16, 7/17, 7/18, 7/19, 7/20, 8/5, 8/6, 8/7, 8/9, 8/10, 8/11, 8/12, 8/13, 8/14, 8/15, 8/16, 8/17, 8/18, 8/19, 8/20, 9/5, 9/6, 9/7, 9/8, 9/10, 9/11, 9/12, 9/13, 9/14, 9/15, 9/16, 9/17, 9/18, 9/19, 9/20, 10/5, 10/6, 10/7, 10/8, 10/9, 10/11, 10/12, 10/13, 10/14, 10/15, 10/16, 10/17, 10/18, 10/19, 10/20, 11/5, 11/6, 11/7, 11/8, 11/9, 11/10, 11/12, 11/13, 11/14, 11/15, 11/16, 11/17, 11/18, 11/19, 11/20, 12/5, 12/6, 12/7, 12/8, 12/9, 12/10, 12/11, 12/13, 12/14, 12/15, 12/16, 12/17, 12/18, 12/19, 12/20, 13/5, 13/6, 13/7, 13/8, 13/9, 13/10, 13/11, 13/12, 13/14, 13/15, 13/16, 13/17, 13/18, 13/19, 13/20, 14/5, 14/6, 14/7, 14/8, 14/9, 14/10, 14/11, 14/12, 14/13, 14/15, 14/16, 14/17, 14/18, 14/19, 14/20, 15/5, 15/6, 15/7, 15/8, 15/9, 15/10, 15/11, 15/12, 15/13, 15/14, 15/16, 15/17, 15/18, 15/19, 15/20, 16/5, 16/6, 16/7, 16/8, 16/9, 16/10, 16/11, 16/12, 16/13, 16/14, 16/15, 16/17, 16/18, 16/19, 16/20, 17/5, 17/6, 17/7, 17/8, 17/9, 17/10, 17/11, 17/12, 17/13, 17/14, 17/15, 17/16, 17/18, 17/19, 17/20, 18/5, 18/6, 18/7, 18/8, 18/9, 18/10, 18/11, 18/12, 18/13, 18/14, 18/15, 18/16, 18/17, 18/19, 18/20, 19/5, 19/6, 19/7, 19/8, 19/9, 19/10, 19/11, 19/12, 19/13, 19/14, 19/15, 19/16, 19/17, 19/18, 19/20, 20/5, 20/6, 20/7, 20/8, 20/9, 20/10, 20/11, 20/12, 20/13, 20/14, 20/15, 20/16, 20/17, 20/18, or 20/19 asymmetric internal loop, wherein the first number is the number of nucleotides contributed to the symmetric internal loop or the asymmetric internal loop from the engineered guide RNA side of the guide-target RNA scaffold and the second number is the number of nucleotides contributed to the symmetric internal loop or the asymmetric internal loop from the target RNA side of the guide-target RNA scaffold. In some embodiments, the first internal loop is an asymmetric internal loop and the second internal loop is a symmetric internal loop. In some embodiments, the first internal loop is an asymmetric internal loop that is a 5/6, 5/7, 5/8, 5/9, 5/10, 5/11, 5/12, 5/13, 5/14, 5/15, 5/16, 5/17, 5/18, 5/19, 5/20, 6/5, 6/7, 6/8, 6/9, 6/10, 6/11, 6/12, 6/13, 6/14, 6/15, 6/16, 6/17, 6/18, 6/19, 6/20, 7/5, 7/6, 7/8, 7/9, 7/10, 7/11, 7/12, 7/13, 7/14, 7/15, 7/16, 7/17, 7/18, 7/19, 7/20, 8/5, 8/6, 8/7, 8/9, 8/10, 8/11, 8/12, 8/13, 8/14, 8/15, 8/16, 8/17, 8/18, 8/19, 8/20, 9/5, 9/6, 9/7, 9/8, 9/10, 9/11, 9/12, 9/13, 9/14, 9/15, 9/16, 9/17, 9/18, 9/19, 9/20, 10/5, 10/6, 10/7, 10/8, 10/9, 10/11, 10/12, 10/13, 10/14, 10/15, 10/16, 10/17, 10/18, 10/19, 10/20, 11/5, 11/6, 11/7, 11/8, 11/9, 11/10, 11/12, 11/13, 11/14, 11/15, 11/16, 11/17, 11/18, 11/19, 11/20, 12/5, 12/6, 12/7, 12/8, 12/9, 12/10, 12/11, 12/13, 12/14, 12/15, 12/16, 12/17, 12/18, 12/19, 12/20, 13/5, 13/6, 13/7, 13/8, 13/9, 13/10, 13/11, 13/12, 13/14, 13/15, 13/16, 13/17, 13/18, 13/19, 13/20, 14/5, 14/6, 14/7, 14/8, 14/9, 14/10, 14/11, 14/12, 14/13, 14/15, 14/16, 14/17, 14/18, 14/19, 14/20, 15/5, 15/6, 15/7, 15/8, 15/9, 15/10, 15/11, 15/12, 15/13, 15/14, 15/16, 15/17, 15/18, 15/19, 15/20, 16/5, 16/6, 16/7, 16/8, 16/9, 16/10, 16/11, 16/12, 16/13, 16/14, 16/15, 16/17, 16/18, 16/19, 16/20, 17/5, 17/6, 17/7, 17/8, 17/9, 17/10, 17/11, 17/12, 17/13, 17/14, 17/15, 17/16, 17/18, 17/19, 17/20, 18/5, 18/6, 18/7, 18/8, 18/9, 18/10, 18/11, 18/12, 18/13, 18/14, 18/15, 18/16, 18/17, 18/19, 18/20, 19/5, 19/6, 19/7, 19/8, 19/9, 19/10, 19/11, 19/12, 19/13, 19/14, 19/15, 19/16, 19/17, 19/18, 19/20, 20/5, 20/6, 20/7, 20/8, 20/9, 20/10, 20/11, 20/12, 20/13, 20/14, 20/15, 20/16, 20/17, 20/18, or 20/19 asymmetric internal loop; and wherein the second internal loop is a symmetric internal loop that is a 5/5, 6/6, 7/7, 8/8, 9/9, 10/10, 11/11, 12/12, 13/13, 14/14, 15/15, 16/16, 17/17, 18/18, 19/19, or 20/20 symmetric internal loop, wherein the first number is the number of nucleotides contributed to the symmetric internal loop or the asymmetric internal loop from the engineered guide RNA side of the guide-target RNA scaffold and the second number is the number of nucleotides contributed to the symmetric internal loop or the asymmetric internal loop from the target RNA side of the guide-target RNA scaffold. In some embodiments, the first internal loop and the second internal loop comprise the same number of bases. In some embodiments, the first internal loop and the second internal loop comprise a different number of bases. In some embodiments, the first internal loop comprises a greater number of bases than the second internal loop. In some embodiments, the second internal loop comprises a greater number of bases than the first internal loop. In some embodiments, the first internal loop and the second internal loop independently comprise at least about 5 bases to at least about 20 bases of the engineered guide RNA and at least about 5 bases to at least about 20 bases of the target RNA. In some embodiments, the engineered guide RNA comprises a cytosine that, when the engineered guide RNA is hybridized to the target RNA, is present in the guide-target RNA scaffold opposite the on-target adenosine that is edited by the RNA editing entity, thereby forming an A/C mismatch in the guide-target RNA scaffold. In some embodiments, the first internal loop and the second internal loop are positioned the same number of bases from the A/C mismatch with respect to the base of the first internal loop and the base of the second internal loop that is most proximal to the A/C mismatch. In some embodiments, the first internal loop is positioned at least about 5 bases away from the A/C mismatch with respect to the base of the first internal loop that is most proximal to the A/C mismatch. In some embodiments, the first internal loop is positioned from about 1 bases away from the A/C mismatch to about 30 bases away from the A/C mismatch with respect to the base of the first internal loop that is most proximal to the A/C mismatch; optionally wherein the first internal loop is positioned 6 bases, 10 bases, 12 bases, or 15 bases away from the A/C mismatch with respect to the base of the first internal loop that is most proximal to the A/C mismatch. In some embodiments, the second internal loop is positioned at least about 12 bases away from the A/C mismatch with respect to the base of the second internal loop that is most proximal to the A/C mismatch. In some embodiments, the second internal loop is positioned from about 12 bases away from the A/C mismatch to about 40 bases away from the A/C mismatch with respect to the base of the second internal loop that is most proximal to the A/C mismatch; optionally wherein the second internal loop is positioned 24 bases, 30 bases, 33 bases, or 34 bases away from the A/C mismatch with respect to the base of the second internal loop that is most proximal to the A/C mismatch. In some embodiments, the target RNA is an mRNA selected from the group consisting of: ABCA4, APP, CFTR, DMPK, DUX4, GAPDH, GBA, GRN, HEXA, LIPA, LRRK2, MAPT, PINK1, PMP22, SERPINA1, SNCA, or SOD1, a fragment of any one of these, and any combination thereof. In some embodiments, the target RNA is ABCA4, and wherein the ABCA4 comprises a target mutation for RNA editing selected from the group consisting of: G6320A; G5714A; G5882A; and any combination thereof. In some embodiments, the first internal loop is positioned from about 5 bases away from the A/C mismatch to about 15 bases away from the A/C mismatch with respect to the base of the first internal loop that is most proximal to the A/C mismatch. In some embodiments, the first internal loop is positioned 15 bases away from the A/C mismatch with respect to the base of the first internal loop that is most proximal to the A/C mismatch. In some embodiments, the second internal loop is positioned from about 12 bases away from the A/C mismatch to about 40 bases away from the A/C mismatch with respect to the base of the second internal loop that is most proximal to the A/C mismatch. In some embodiments, the second internal loop is positioned 33 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 1-105, 2729-2761, or 2772-2843. In some embodiments, the target RNA is APP, and wherein a target mutation is introduced into the APP RNA, wherein a polypeptide encoded by the APP RNA after modification comprises a polypeptide mutation selected from the group consisting of: K670E, K670R, K670G, M671V, A673V, A673T, D672G, E682G, H684R, K687R, K687E, K687G, I712X, T714X, and any combination thereof. In some embodiments, the first internal loop is positioned from about 5 bases away from the A/C mismatch to about 20 bases away from the A/C mismatch with respect to the base of the first internal loop that is most proximal to the A/C mismatch. In some embodiments, the first internal loop is positioned 10 bases away from the A/C mismatch with respect to the base of the first internal loop that is most proximal to the A/C mismatch. In some embodiments, the second internal loop is positioned from about 15 bases away from the A/C mismatch to about 40 bases away from the A/C mismatch with respect to the base of the second internal loop that is most proximal to the A/C mismatch. In some embodiments, the second internal loop is positioned 33 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 112-114. In some embodiments, the target RNA is SERPINA1, and wherein the SERPINA encodes a polypeptide that comprises an E342K mutation. In some embodiments, the first internal loop is positioned from about 5 bases away from the A/C mismatch to about 20 bases away from the A/C mismatch with respect to the base of the first internal loop that is most proximal to the A/C mismatch. In some embodiments, the first internal loop is positioned 12 bases away from the A/C mismatch with respect to the base of the first internal loop that is most proximal to the A/C mismatch. In some embodiments, the second internal loop is positioned from about 12 bases away from the A/C mismatch to about 40 bases away from the A/C mismatch with respect to the base of the second internal loop that is most proximal to the A/C mismatch. In some embodiments, the second internal loop is positioned 24 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 2762-2768 or 3083-3086. In some embodiments, the target RNA is LRRK2, and wherein the LRRK2 encodes a polypeptide with a polypeptide mutation selected from the group consisting of: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q1111H, 11122V, A1151T, L1165P, 11192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, Q2490NfsX3, and any combination thereof. In some embodiments, the first internal loop is positioned from about 7 bases away from the A/C mismatch to about 30 bases away from the A/C mismatch with respect to the base of the first internal loop that is most proximal to the A/C mismatch. In some embodiments, the first internal loop is positioned 10 bases away from the A/C mismatch with respect to the base of the first internal loop that is most proximal to the A/C mismatch. In some embodiments, the second internal loop is positioned from about 18 bases away from the A/C mismatch to about 34 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 118-167, 2686-2728, 2769-2771, 2844-3078, or 3081-3082. In some embodiments, the target RNA is SNCA, and wherein the SNCA comprises a target mutation for RNA editing selected from the group consisting of: translation initiation site (TIS) ATG to GTG in Codon 1 and Codon 5; AUG at position 265 in Exon 2. In some embodiments, the first internal loop is positioned from about 6 bases away from the A/C mismatch to about 20 bases away from the A/C mismatch with respect to the base of the first internal loop that is most proximal to the A/C mismatch. In some embodiments, the first internal loop is positioned 6 bases away from the A/C mismatch with respect to the base of the first internal loop that is most proximal to the A/C mismatch. In some embodiments, the second internal loop is positioned from about 15 bases away from the A/C mismatch to about 38 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 2480-2681. In some embodiments, the target RNA is MAPT, and wherein the MAPT comprises a target mutation for RNA editing at the translation initiation site (TIS). In some embodiments, the first internal loop is positioned from about 5 bases away from the A/C mismatch to about 15 bases away from the A/C mismatch with respect to the base of the first internal loop that is most proximal to the A/C mismatch. In some embodiments, the first internal loop is positioned 15 bases away from the A/C mismatch with respect to the base of the first internal loop that is most proximal to the A/C mismatch. In some embodiments, the second internal loop is positioned from about 12 bases away from the A/C mismatch to about 40 bases away from the A/C mismatch with respect to the base of the second internal loop that is most proximal to the A/C mismatch. In some embodiments, the second internal loop is positioned 33 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 115-117, 1519-2479 or 2682-2685. In some embodiments, the target RNA is DUX4. In some embodiments, the first internal loop is positioned from about 1 base away from the A/C mismatch to about 20 bases away from the A/C mismatch with respect to the base of the first internal loop that is most proximal to the A/C mismatch. In some embodiments, the first internal loop is positioned 6 bases away from the A/C mismatch with respect to the base of the first internal loop that is most proximal to the A/C mismatch. In some embodiments, the second internal loop is positioned from about 15 bases away from the A/C mismatch to about 40 bases away from the A/C mismatch with respect to the base of the second internal loop that is most proximal to the A/C mismatch. In some embodiments, the second internal loop is positioned 33 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 172-1518. In some embodiments, the target RNA is GRN. In some embodiments, the first internal loop is positioned from about 5 bases away from the A/C mismatch to about 20 bases away from the A/C mismatch with respect to the base of the first internal loop that is most proximal to the A/C mismatch. In some embodiments, the first internal loop is positioned 12 bases away from the A/C mismatch with respect to the base of the first internal loop that is most proximal to the A/C mismatch. In some embodiments, the second internal loop is positioned from about 18 bases away from the A/C mismatch to about 38 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch. In some embodiments, the engineered guide RNA comprises a length of at least about 60 bases. In some embodiments, the engineered guide RNA comprises a length of about 65 bases to about 150 bases. In some embodiments, the at least one structural feature comprises a bulge. In some embodiments, the bulge comprises an asymmetric bulge. In some embodiments, the bulge comprises a symmetric bulge. In some embodiments, the bulge independently comprises about 1 base to about 4 bases of the engineered guide RNA and about 0 bases to about 4 bases of the target RNA. In some embodiments, the at least one structural feature comprises an internal loop. In some embodiments, the internal loop comprises an asymmetric internal loop. In some embodiments, the internal loop comprises a symmetric internal loop. In some embodiments, the internal loop independently comprises about 5 to about 10 bases of either the engineered guide RNA or the target RNA. In some embodiments, the at least one structural feature comprises a hairpin. In some embodiments, the hairpin comprises a length of about 3 bases to about 15 bases in length. In some embodiments, the RNA editing entity is endogenous to a mammalian cell. In some embodiments, the RNA editing entity is an adenosine deaminase acting on RNA (ADAR) enzyme, a catalytically active fragment thereof, or a fusion polypeptide thereof. In some embodiments, the RNA editing entity is the ADAR enzyme. In some embodiments, the ADAR enzyme comprises human ADAR (hADAR). In some embodiments, the ADAR enzyme comprises ADAR1 or ADAR2. In some embodiments, the target RNA is an mRNA or pre-mRNA. In some embodiments, the engineered guide RNA further comprises at least one chemical modification. In some embodiments, the at least one chemical modification comprises a 2′-O-methyl group on a ribose sugar of a nucleotide of the engineered guide RNA. In some embodiments, the at least one chemical modification comprises a phosphothioate modification of a backbone of the engineered guide RNA. In some embodiments, the engineered guide comprises a total polynucleotide length of at least about 65 bases. In some embodiments, the engineered guide comprises a total polynucleotide length range of about 65 bases to about 100 bases. In some embodiments, the engineered guide RNA is a circular guide RNA.

Also disclosed herein is an engineered guide RNA or a polynucleotide sequence encoding the engineered guide RNA, wherein upon hybridization, the engineered guide RNA and the sequence of the target RNA form a guide-target RNA scaffold, wherein the guide-target RNA scaffold comprises: (i) a micro-footprint that comprises at least one structural feature selected from the group consisting of: a bulge, an internal loop, a mismatch, a wobble base pair, a hairpin, and any combination thereof, wherein, upon contacting the guide-target RNA scaffold with an RNA editing entity, the RNA editing entity edits an on-target adenosine in the target RNA within the guide-target RNA scaffold; and (ii) a barbell macro-footprint that comprises a first internal loop that is 5′ of the micro-footprint and a second internal loop that is 3′ of the micro-footprint, wherein the barbell macro-footprint facilitates an increase in the amount of the editing of the on-target adenosine in the target RNA, relative to an otherwise comparable engineered guide RNA lacking the barbell macro-footprint. In some embodiments, the first internal loop and the second internal loop facilitate a decrease in the amount of off-target adenosine editing in the target RNA, relative to an otherwise comparable engineered guide RNA lacking the first internal loop and the second internal loop. In some embodiments, the first internal loop is a symmetric internal loop and the second internal loop is a symmetric internal loop. In some embodiments, the first internal loop and the second internal loop are symmetric internal loops that independently are 5/5, 6/6, 7/7, 8/8, 9/9, 10/10, 11/11, 12/12, 13/13, 14/14, 15/15, 16/16, 17/17, 18/18, 19/19, or 20/20 symmetric internal loops, wherein the first number is the number of nucleotides contributed to the symmetric internal loop from the engineered guide RNA side of the guide-target RNA scaffold and the second number is the number of nucleotides contributed to the symmetric internal loop from the target RNA side of the guide-target RNA scaffold. In some embodiments, the first internal loop is an asymmetric internal loop and the second internal loop is an asymmetric internal loop. In some embodiments, the first internal loop and the second internal loop are asymmetric internal loops that independently are 5/6, 5/7, 5/8, 5/9, 5/10, 5/11, 5/12, 5/13, 5/14, 5/15, 5/16, 5/17, 5/18, 5/19, 5/20, 6/5, 6/7, 6/8, 6/9, 6/10, 6/11, 6/12, 6/13, 6/14, 6/15, 6/16, 6/17, 6/18, 6/19, 6/20, 7/5, 7/6, 7/8, 7/9, 7/10, 7/11, 7/12, 7/13, 7/14, 7/15, 7/16, 7/17, 7/18, 7/19, 7/20, 8/5, 8/6, 8/7, 8/9, 8/10, 8/11, 8/12, 8/13, 8/14, 8/15, 8/16, 8/17, 8/18, 8/19, 8/20, 9/5, 9/6, 9/7, 9/8, 9/10, 9/11, 9/12, 9/13, 9/14, 9/15, 9/16, 9/17, 9/18, 9/19, 9/20, 10/5, 10/6, 10/7, 10/8, 10/9, 10/11, 10/12, 10/13, 10/14, 10/15, 10/16, 10/17, 10/18, 10/19, 10/20, 11/5, 11/6, 11/7, 11/8, 11/9, 11/10, 11/12, 11/13, 11/14, 11/15, 11/16, 11/17, 11/18, 11/19, 11/20, 12/5, 12/6, 12/7, 12/8, 12/9, 12/10, 12/11, 12/13, 12/14, 12/15, 12/16, 12/17, 12/18, 12/19, 12/20, 13/5, 13/6, 13/7, 13/8, 13/9, 13/10, 13/11, 13/12, 13/14, 13/15, 13/16, 13/17, 13/18, 13/19, 13/20, 14/5, 14/6, 14/7, 14/8, 14/9, 14/10, 14/11, 14/12, 14/13, 14/15, 14/16, 14/17, 14/18, 14/19, 14/20, 15/5, 15/6, 15/7, 15/8, 15/9, 15/10, 15/11, 15/12, 15/13, 15/14, 15/16, 15/17, 15/18, 15/19, 15/20, 16/5, 16/6, 16/7, 16/8, 16/9, 16/10, 16/11, 16/12, 16/13, 16/14, 16/15, 16/17, 16/18, 16/19, 16/20, 17/5, 17/6, 17/7, 17/8, 17/9, 17/10, 17/11, 17/12, 17/13, 17/14, 17/15, 17/16, 17/18, 17/19, 17/20, 18/5, 18/6, 18/7, 18/8, 18/9, 18/10, 18/11, 18/12, 18/13, 18/14, 18/15, 18/16, 18/17, 18/19, 18/20, 19/5, 19/6, 19/7, 19/8, 19/9, 19/10, 19/11, 19/12, 19/13, 19/14, 19/15, 19/16, 19/17, 19/18, 19/20, 20/5, 20/6, 20/7, 20/8, 20/9, 20/10, 20/11, 20/12, 20/13, 20/14, 20/15, 20/16, 20/17, 20/18, or 20/19 asymmetric internal loops, wherein the first number is the number of nucleotides contributed to the asymmetric internal loop from the engineered guide RNA side of the guide-target RNA scaffold and the second number is the number of nucleotides contributed to the asymmetric internal loop from the target RNA side of the guide-target RNA scaffold. In some embodiments, the first internal loop is a symmetric internal loop and the second internal loop is an asymmetric internal loop. In some embodiments, the first internal loop is a symmetric internal loop that is a 5/5, 6/6, 7/7, 8/8, 9/9, 10/10, 11/11, 12/12, 13/13, 14/14, 15/15, 16/16, 17/17, 18/18, 19/19, or 20/20 symmetric internal loop; and wherein the second internal loop is an asymmetric internal loop that is a 5/6, 5/7, 5/8, 5/9, 5/10, 5/11, 5/12, 5/13, 5/14, 5/15, 5/16, 5/17, 5/18, 5/19, 5/20, 6/5, 6/7, 6/8, 6/9, 6/10, 6/11, 6/12, 6/13, 6/14, 6/15, 6/16, 6/17, 6/18, 6/19, 6/20, 7/5, 7/6, 7/8, 7/9, 7/10, 7/11, 7/12, 7/13, 7/14, 7/15, 7/16, 7/17, 7/18, 7/19, 7/20, 8/5, 8/6, 8/7, 8/9, 8/10, 8/11, 8/12, 8/13, 8/14, 8/15, 8/16, 8/17, 8/18, 8/19, 8/20, 9/5, 9/6, 9/7, 9/8, 9/10, 9/11, 9/12, 9/13, 9/14, 9/15, 9/16, 9/17, 9/18, 9/19, 9/20, 10/5, 10/6, 10/7, 10/8, 10/9, 10/11, 10/12, 10/13, 10/14, 10/15, 10/16, 10/17, 10/18, 10/19, 10/20, 11/5, 11/6, 11/7, 11/8, 11/9, 11/10, 11/12, 11/13, 11/14, 11/15, 11/16, 11/17, 11/18, 11/19, 11/20, 12/5, 12/6, 12/7, 12/8, 12/9, 12/10, 12/11, 12/13, 12/14, 12/15, 12/16, 12/17, 12/18, 12/19, 12/20, 13/5, 13/6, 13/7, 13/8, 13/9, 13/10, 13/11, 13/12, 13/14, 13/15, 13/16, 13/17, 13/18, 13/19, 13/20, 14/5, 14/6, 14/7, 14/8, 14/9, 14/10, 14/11, 14/12, 14/13, 14/15, 14/16, 14/17, 14/18, 14/19, 14/20, 15/5, 15/6, 15/7, 15/8, 15/9, 15/10, 15/11, 15/12, 15/13, 15/14, 15/16, 15/17, 15/18, 15/19, 15/20, 16/5, 16/6, 16/7, 16/8, 16/9, 16/10, 16/11, 16/12, 16/13, 16/14, 16/15, 16/17, 16/18, 16/19, 16/20, 17/5, 17/6, 17/7, 17/8, 17/9, 17/10, 17/11, 17/12, 17/13, 17/14, 17/15, 17/16, 17/18, 17/19, 17/20, 18/5, 18/6, 18/7, 18/8, 18/9, 18/10, 18/11, 18/12, 18/13, 18/14, 18/15, 18/16, 18/17, 18/19, 18/20, 19/5, 19/6, 19/7, 19/8, 19/9, 19/10, 19/11, 19/12, 19/13, 19/14, 19/15, 19/16, 19/17, 19/18, 19/20, 20/5, 20/6, 20/7, 20/8, 20/9, 20/10, 20/11, 20/12, 20/13, 20/14, 20/15, 20/16, 20/17, 20/18, or 20/19 asymmetric internal loop, wherein the first number is the number of nucleotides contributed to the symmetric internal loop or the asymmetric internal loop from the engineered guide RNA side of the guide-target RNA scaffold and the second number is the number of nucleotides contributed to the symmetric internal loop or the asymmetric internal loop from the target RNA side of the guide-target RNA scaffold. In some embodiments, the first internal loop is an asymmetric internal loop and the second internal loop is a symmetric internal loop. In some embodiments, the first internal loop is an asymmetric internal loop that is a 5/6, 5/7, 5/8, 5/9, 5/10, 5/11, 5/12, 5/13, 5/14, 5/15, 5/16, 5/17, 5/18, 5/19, 5/20, 6/5, 6/7, 6/8, 6/9, 6/10, 6/11, 6/12, 6/13, 6/14, 6/15, 6/16, 6/17, 6/18, 6/19, 6/20, 7/5, 7/6, 7/8, 7/9, 7/10, 7/11, 7/12, 7/13, 7/14, 7/15, 7/16, 7/17, 7/18, 7/19, 7/20, 8/5, 8/6, 8/7, 8/9, 8/10, 8/11, 8/12, 8/13, 8/14, 8/15, 8/16, 8/17, 8/18, 8/19, 8/20, 9/5, 9/6, 9/7, 9/8, 9/10, 9/11, 9/12, 9/13, 9/14, 9/15, 9/16, 9/17, 9/18, 9/19, 9/20, 10/5, 10/6, 10/7, 10/8, 10/9, 10/11, 10/12, 10/13, 10/14, 10/15, 10/16, 10/17, 10/18, 10/19, 10/20, 11/5, 11/6, 11/7, 11/8, 11/9, 11/10, 11/12, 11/13, 11/14, 11/15, 11/16, 11/17, 11/18, 11/19, 11/20, 12/5, 12/6, 12/7, 12/8, 12/9, 12/10, 12/11, 12/13, 12/14, 12/15, 12/16, 12/17, 12/18, 12/19, 12/20, 13/5, 13/6, 13/7, 13/8, 13/9, 13/10, 13/11, 13/12, 13/14, 13/15, 13/16, 13/17, 13/18, 13/19, 13/20, 14/5, 14/6, 14/7, 14/8, 14/9, 14/10, 14/11, 14/12, 14/13, 14/15, 14/16, 14/17, 14/18, 14/19, 14/20, 15/5, 15/6, 15/7, 15/8, 15/9, 15/10, 15/11, 15/12, 15/13, 15/14, 15/16, 15/17, 15/18, 15/19, 15/20, 16/5, 16/6, 16/7, 16/8, 16/9, 16/10, 16/11, 16/12, 16/13, 16/14, 16/15, 16/17, 16/18, 16/19, 16/20, 17/5, 17/6, 17/7, 17/8, 17/9, 17/10, 17/11, 17/12, 17/13, 17/14, 17/15, 17/16, 17/18, 17/19, 17/20, 18/5, 18/6, 18/7, 18/8, 18/9, 18/10, 18/11, 18/12, 18/13, 18/14, 18/15, 18/16, 18/17, 18/19, 18/20, 19/5, 19/6, 19/7, 19/8, 19/9, 19/10, 19/11, 19/12, 19/13, 19/14, 19/15, 19/16, 19/17, 19/18, 19/20, 20/5, 20/6, 20/7, 20/8, 20/9, 20/10, 20/11, 20/12, 20/13, 20/14, 20/15, 20/16, 20/17, 20/18, or 20/19 asymmetric internal loop; and wherein the second internal loop is a symmetric internal loop that is a 5/5, 6/6, 7/7, 8/8, 9/9, 10/10, 11/11, 12/12, 13/13, 14/14, 15/15, 16/16, 17/17, 18/18, 19/19, or 20/20 symmetric internal loop, wherein the first number is the number of nucleotides contributed to the symmetric internal loop or the asymmetric internal loop from the engineered guide RNA side of the guide-target RNA scaffold and the second number is the number of nucleotides contributed to the symmetric internal loop or the asymmetric internal loop from the target RNA side of the guide-target RNA scaffold. In some embodiments, the first internal loop and the second internal loop comprise the same number of bases. In some embodiments, the first internal loop and the second internal loop comprise a different number of bases. In some embodiments, the first internal loop comprises a greater number of bases than the second internal loop. In some embodiments, the second internal loop comprises a greater number of bases than the first internal loop. In some embodiments, the first internal loop and the second internal loop independently comprise at least about 5 bases to at least about 20 bases of the engineered guide RNA and at least about 5 bases to at least about 20 bases of the target RNA. In some embodiments, the engineered guide RNA comprises a cytosine that, when the engineered guide RNA is hybridized to the target RNA, is present in the guide-target RNA scaffold opposite the on-target adenosine that is edited by the RNA editing entity, thereby forming an A/C mismatch in the guide-target RNA scaffold. In some embodiments, the first internal loop and the second internal loop are positioned the same number of bases from the A/C mismatch with respect to the base of the first internal loop and the base of the second internal loop that is most proximal to the A/C mismatch. In some embodiments, the first internal loop is positioned at least about 5 bases away from the A/C mismatch with respect to the base of the first internal loop that is most proximal to the A/C mismatch. In some embodiments, the first internal loop is positioned from about 1 bases away from the A/C mismatch to about 30 bases away from the A/C mismatch with respect to the base of the first internal loop that is most proximal to the A/C mismatch; optionally wherein the first internal loop is positioned 6 bases, 10 bases, 12 bases, or 15 bases away from the A/C mismatch with respect to the base of the first internal loop that is most proximal to the A/C mismatch. In some embodiments, the second internal loop is positioned at least about 12 bases away from the A/C mismatch with respect to the base of the second internal loop that is most proximal to the A/C mismatch. In some embodiments, the second internal loop is positioned from about 12 bases away from the A/C mismatch to about 40 bases away from the A/C mismatch with respect to the base of the second internal loop that is most proximal to the A/C mismatch; optionally wherein the second internal loop is positioned 24 bases, 30 bases, 33 bases, or 34 bases away from the A/C mismatch with respect to the base of the second internal loop that is most proximal to the A/C mismatch. In some embodiments, the target RNA is an mRNA selected from the group consisting of: ABCA4, APP, CFTR, DMPK, DUX4, GAPDH, GBA, GRN, HEXA, LIPA, LRRK2, MAPT, PINK1, PMP22, SERPINA1, SNCA, or SOD1, a fragment of any one of these, and any combination thereof. In some embodiments, the target RNA is ABCA4, and wherein the ABCA4 comprises a target mutation for RNA editing selected from the group consisting of: G6320A; G5714A; G5882A; and any combination thereof. In some embodiments, the first internal loop is positioned from about 5 bases away from the A/C mismatch to about 15 bases away from the A/C mismatch with respect to the base of the first internal loop that is most proximal to the A/C mismatch. In some embodiments, the first internal loop is positioned 15 bases away from the A/C mismatch with respect to the base of the first internal loop that is most proximal to the A/C mismatch. In some embodiments, the second internal loop is positioned from about 12 bases away from the A/C mismatch to about 40 bases away from the A/C mismatch with respect to the base of the second internal loop that is most proximal to the A/C mismatch. In some embodiments, the second internal loop is positioned 33 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 1-105, 2729-2761, or 2772-2843. In some embodiments, the target RNA is APP, and wherein a target mutation is introduced into the APP RNA, wherein a polypeptide encoded by the APP RNA after modification comprises a polypeptide mutation selected from the group consisting of: K670E, K670R, K670G, M671V, A673V, A673T, D672G, E682G, H684R, K687R, K687E, K687G, I712X, T714X, and any combination thereof. In some embodiments, the first internal loop is positioned from about 5 bases away from the A/C mismatch to about 20 bases away from the A/C mismatch with respect to the base of the first internal loop that is most proximal to the A/C mismatch. In some embodiments, the first internal loop is positioned 10 bases away from the A/C mismatch with respect to the base of the first internal loop that is most proximal to the A/C mismatch. In some embodiments, the second internal loop is positioned from about 15 bases away from the A/C mismatch to about 40 bases away from the A/C mismatch with respect to the base of the second internal loop that is most proximal to the A/C mismatch. In some embodiments, the second internal loop is positioned 33 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 112-114. In some embodiments, the target RNA is SERPINA1, and wherein the SERPINA encodes a polypeptide that comprises an E342K mutation. In some embodiments, the first internal loop is positioned from about 5 bases away from the A/C mismatch to about 20 bases away from the A/C mismatch with respect to the base of the first internal loop that is most proximal to the A/C mismatch. In some embodiments, the first internal loop is positioned 12 bases away from the A/C mismatch with respect to the base of the first internal loop that is most proximal to the A/C mismatch. In some embodiments, the second internal loop is positioned from about 12 bases away from the A/C mismatch to about 40 bases away from the A/C mismatch with respect to the base of the second internal loop that is most proximal to the A/C mismatch. In some embodiments, the second internal loop is positioned 24 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 2762-2768 or 3083-3086. In some embodiments, the target RNA is LRRK2, and wherein the LRRK2 encodes a polypeptide with a polypeptide mutation selected from the group consisting of: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, Q2490NfsX3, and any combination thereof. In some embodiments, the first internal loop is positioned from about 7 bases away from the A/C mismatch to about 30 bases away from the A/C mismatch with respect to the base of the first internal loop that is most proximal to the A/C mismatch. In some embodiments, the first internal loop is positioned 10 bases away from the A/C mismatch with respect to the base of the first internal loop that is most proximal to the A/C mismatch. In some embodiments, the second internal loop is positioned from about 18 bases away from the A/C mismatch to about 34 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 118-167, 2686-2728, 2769-2771, 2844-3078, or 3081-3082. In some embodiments, the target RNA is SNCA, and wherein the SNCA comprises a target mutation for RNA editing selected from the group consisting of: translation initiation site (TIS) ATG to GTG in Codon 1 and Codon 5; AUG at position 265 in Exon 2. In some embodiments, the first internal loop is positioned from about 6 bases away from the A/C mismatch to about 20 bases away from the A/C mismatch with respect to the base of the first internal loop that is most proximal to the A/C mismatch. In some embodiments, the first internal loop is positioned 6 bases away from the A/C mismatch with respect to the base of the first internal loop that is most proximal to the A/C mismatch. In some embodiments, the second internal loop is positioned from about 15 bases away from the A/C mismatch to about 38 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 2480-2681. In some embodiments, the target RNA is MAPT, and wherein the MAPT comprises a target mutation for RNA editing at the translation initiation site (TIS). In some embodiments, the first internal loop is positioned from about 5 bases away from the A/C mismatch to about 15 bases away from the A/C mismatch with respect to the base of the first internal loop that is most proximal to the A/C mismatch. In some embodiments, the first internal loop is positioned 15 bases away from the A/C mismatch with respect to the base of the first internal loop that is most proximal to the A/C mismatch. In some embodiments, the second internal loop is positioned from about 12 bases away from the A/C mismatch to about 40 bases away from the A/C mismatch with respect to the base of the second internal loop that is most proximal to the A/C mismatch. In some embodiments, the second internal loop is positioned 33 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 115-117, 1519-2479 or 2682-2685. In some embodiments, the target RNA is DUX4. In some embodiments, the first internal loop is positioned from about 1 base away from the A/C mismatch to about 20 bases away from the A/C mismatch with respect to the base of the first internal loop that is most proximal to the A/C mismatch. In some embodiments, the first internal loop is positioned 6 bases away from the A/C mismatch with respect to the base of the first internal loop that is most proximal to the A/C mismatch. In some embodiments, the second internal loop is positioned from about 15 bases away from the A/C mismatch to about 40 bases away from the A/C mismatch with respect to the base of the second internal loop that is most proximal to the A/C mismatch. In some embodiments, the second internal loop is positioned 33 bases away from the A/C mismatch with respect to the base of the second internal loop that is most proximal to the A/C mismatch. In some embodiments, the engineered guide RNA comprises a polynucleotide sequence with at least 80%, 85%, 90%, 92%, 95%, 97%, or 99% sequence identity to any one of SEQ ID NO: 172-1518. In some embodiments, the target RNA is GRN. In some embodiments, the first internal loop is positioned from about 5 bases away from the A/C mismatch to about 20 bases away from the A/C mismatch with respect to the base of the first internal loop that is most proximal to the A/C mismatch. In some embodiments, the first internal loop is positioned 12 bases away from the A/C mismatch with respect to the base of the first internal loop that is most proximal to the A/C mismatch. In some embodiments, the second internal loop is positioned from about 18 bases away from the A/C mismatch to about 38 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch. In some embodiments, the engineered guide RNA comprises a length of at least about 60 bases. In some embodiments, the engineered guide RNA comprises a length of about 65 bases to about 150 bases. In some embodiments, the at least one structural feature comprises a bulge. In some embodiments, the bulge comprises an asymmetric bulge. In some embodiments, the bulge comprises a symmetric bulge. In some embodiments, the bulge independently comprises about 1 base to about 4 bases of the engineered guide RNA and about 0 bases to about 4 bases of the target RNA. In some embodiments, the at least one structural feature comprises an internal loop. In some embodiments, the internal loop comprises an asymmetric internal loop. In some embodiments, the internal loop comprises a symmetric internal loop. In some embodiments, the internal loop independently comprises about 5 to about 10 bases of either the engineered guide RNA or the target RNA. In some embodiments, the at least one structural feature comprises a hairpin. In some embodiments, the hairpin comprises a length of about 3 bases to about 15 bases in length. In some embodiments, the RNA editing entity is endogenous to a mammalian cell. In some embodiments, the RNA editing entity is an adenosine deaminase acting on RNA (ADAR) enzyme, a catalytically active fragment thereof, or a fusion polypeptide thereof. In some embodiments, the RNA editing entity is the ADAR enzyme. In some embodiments, the ADAR enzyme comprises human ADAR (hADAR). In some embodiments, the ADAR enzyme comprises ADAR1 or ADAR2. In some embodiments, the target RNA is an mRNA or pre-mRNA. In some embodiments, the engineered guide RNA further comprises at least one chemical modification. In some embodiments, the at least one chemical modification comprises a 2′-O-methyl group on a ribose sugar of a nucleotide of the engineered guide RNA. In some embodiments, the at least one chemical modification comprises a phosphothioate modification of a backbone of the engineered guide RNA. In some embodiments, the engineered guide comprises a total polynucleotide length of at least about 65 bases. In some embodiments, the engineered guide comprises a total polynucleotide length range of about 65 bases to about 100 bases. In some embodiments, the engineered guide RNA is a circular guide RNA.

Also disclosed herein is a polynucleotide encoding an engineered guide RNA as described herein.

Also disclosed herein is a delivery vehicle comprising an engineered guide RNA as described herein or a polynucleotide as described herein. In some embodiments, the delivery vehicle is selected from the group consisting of: a delivery vector, a liposome, a particle, and any combination thereof. In some embodiments, the delivery vehicle is a delivery vector, wherein the delivery vector comprises a viral vector. In some embodiments, the viral vector comprises an adeno-associated viral (AAV) vector or derivative thereof. In some embodiments, the AAV vector or derivative thereof is from an adeno-associated virus having a serotype selected from the group consisting of: AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, and AAVhu68. In some embodiments, the AAV vector or derivative thereof is a recombinant AAV (rAAV) vector, a hybrid AAV vector, a chimeric AAV vector, a single stranded AAV (ss-AAV), a self-complementary AAV (scAAV) vector, or any combination thereof.

Also disclosed herein is a pharmaceutical composition comprising: (a) an engineered guide RNA as described herein, a polynucleotide as described herein, or a delivery vehicle as described herein; and (b) a pharmaceutically acceptable excipient, diluent, or carrier.

Also disclosed herein is a method of treating a disease in a subject in need thereof, the method comprising: administering to the subject an effective amount of an engineered guide RNA as described herein, a polynucleotide as described herein, a delivery vehicle as described herein, or a pharmaceutical composition as described herein, wherein the effective amount is sufficient to treat the disease in the subject. In some embodiments, the administering is intrathecal, intraocular, intravitreal, retinal, intravenous, intramuscular, intraventricular, intracerebral, intracerebellar, intracerebroventricular, intraperenchymal, subcutaneous, or a combination thereof. In some embodiments, the disease is macular degeneration. In some embodiments, the disease is Stargardt Disease. In some embodiments, the disease comprises a neurological disease. In some embodiments, the neurological disease comprises Parkinson's disease, Alzheimer's disease, a Tauopathy, or dementia. In some embodiments, the disease comprises a liver disease. In some embodiments, the liver disease comprises liver cirrhosis. In some embodiments, the liver disease comprises alpha-1 antitrypsin deficiency (AAT deficiency). In some embodiments, the target RNA is ABCA4. In some embodiments, the ABCA4 comprises a target mutation for RNA editing selected from the group consisting of: G6320A; G5714A; G5882A; and any combination thereof. In some embodiments, the subject is diagnosed with the disease.

Also disclosed herein is a method of improving an editing efficiency of an engineered guide RNA configured to facilitate an edit of an adenosine in a target RNA via an RNA editing entity, wherein upon hybridization, the engineered guide RNA and the sequence of the target RNA form a guide-target RNA scaffold, wherein the guide-target RNA scaffold comprises: a region that comprises at least one structural feature selected from the group consisting of: a bulge, a wobble base pair, an internal loop, a mismatch, a hairpin, and any combination thereof, wherein, upon contacting the guide-target RNA scaffold with an RNA editing entity, the RNA editing entity edits an on-target adenosine in the target RNA within the guide-target RNA scaffold, the method comprising inserting a first sequence and a second sequence into the engineered guide RNA that, when the engineered guide RNA is hybridized to the target RNA, form a first internal loop and a second internal loop, respectively, on opposing ends of the region of the guide-target RNA scaffold; wherein the first internal loop and the second internal loop facilitate an increase in the amount of the editing of the on-target adenosine in the target RNA relative to an otherwise comparable engineered guide RNA lacking the first internal loop and the second internal loop, thereby improving the editing efficiency of the engineered guide RNA.

Also disclosed herein is an engineered guide RNA as described herein, a polynucleotide as described herein, a delivery vehicle as described herein, or a pharmaceutical composition as described herein, for use in treatment of a disease. In some embodiments, the medicament is administered via the intrathecal, intraocular, intravitreal, retinal, intravenous, intramuscular, intraventricular, intracerebral, intracerebellar, intracerebroventricular, intraperenchymal, or subcutaneous routes or a combination thereof. In some embodiments, the disease is macular degeneration. In some embodiments, the disease is Stargardt Disease. In some embodiments, the disease comprises a neurological disease. In some embodiments, the neurological disease comprises Parkinson's disease, Alzheimer's disease, a Tauopathy, or dementia. In some embodiments, the disease comprises a liver disease. In some embodiments, the liver disease comprises liver cirrhosis. In some embodiments, the liver disease comprises alpha-1 antitrypsin deficiency (AAT deficiency). In some embodiments, the target RNA is ABCA4. In some embodiments, the ABCA4 comprises a target mutation for RNA editing selected from the group consisting of: G6320A; G5714A; G5882A; and any combination thereof.

### BRIEF DESCRIPTION OF THE DRAWINGS

Novel features of the present disclosure are set forth with particularity in the appended claims. A better understanding of the features and advantages of the present disclosure will be obtained by reference to the following detailed description that sets forth illustrative embodiments and the accompanying drawings of which:

FIG. 1 shows various guide RNA engineering. (1) Micro-footprint positioning; (2) Micro-footprint within a macro-footprint (left bell coordinates: LB, generally with the coordinates recited with respect to mismatch; right bell coordinates: RB, generally with the coordinates recited with respect to mismatch); and (3) Guide shortening.

FIG. 2 shows an exemplary micro-footprint having a (+) RNA editing region and a (−) RNA editing region with complementarity within a macro-footprint, which includes two internal symmetrical loops, each of which flanking opposing ends of the micro-footprint. The left/first internal symmetrical loop is −6 bases from the target adenosine (A-C) mismatch, while the right/second internal symmetrical loop is +30 bases from the A-C mismatch.

FIG. 3 shows guide-target RNA scaffolds formed by engineered RNA guides of varying lengths of ABCA4 edited by ADAR1 (in duplicate), where 0.85.65 guides for both biological replicates demonstrated an RNA editing percentage of about 15%. As shown in FIG. 3, the 0.85.65 guide drives higher RNA editing efficiency and also mediates specificity, relative to comparable guides screened in FIG. 3.

FIG. 4 shows varying guide-target RNA scaffolds formed by engineered RNA guides, where the second internal symmetrical loop (or right bell) is located at varying distances from the target adenosine to be edited while the left barbell is left at position −5 relative to the mismatch. Guides 0.85.65 (−5, +33) and 0.85.65 (−5, +27) demonstrated an RNA editing percentage of more than about 15%.

FIG. 5 shows the ADAR profiles depicting ADAR-mediated RNA editing percentage for each of the engineered RNA guides (0.85.65 (−5, +33) and 0.85.65 (−5,+27)) and control without the first and second internal symmetrical loops (0.85.65).

FIG. 6 shows varying guide-target RNA scaffolds formed by engineered RNA guides for 0.85.65, where the first internal symmetrical loop (or left bell) is located at varying distances from the mismatch.

FIG. 7 shows varying guide-target RNA scaffolds formed by engineered RNA guides, where the first internal symmetrical loop (or left bell) is located at varying distances from the mismatch. Exemplary guides 0.85.65 (−10, +33) and 0.85.65 (−9,+33) demonstrate an ADAR-mediated RNA editing percentage of more than about 30%.

FIG. 8 shows the target positions and ADAR-mediated RNA editing percentage for each of the exemplary engineered RNA guides (0.85.65 (−5, +33), 0.85.65 (−9,+33), and 0.85.65 (−10,+33), where GAA is edited at the second base position, which is identified as the zero target position denoted as “0” on the x-axis. Each of the exemplary guides demonstrate an ADAR-mediated RNA editing percentage of more than about 40% at the zero target position.

FIG. 9 shows specific biological replicate and average RNA editing percentages for varying guide-target RNA scaffolds formed by engineered RNA guides via ADAR, where exemplary guides have an ADAR-mediated RNA editing percentage of about 20% or greater (e.g., 0.100.65, 0.100.67, 0.100.70, 0.100.72) As shown in FIG. 9, the positioning of the mismatch modulates RNA editing, with the 100.70 guide representing a superior configuration.).

FIG. 10 shows varying guide-target RNA scaffolds formed by engineered RNA guides, where the second internal symmetrical loop (or right bell) is located at varying distances from the mismatch based on guide 0.100.70.

FIG. 11 shows exemplary guide-target RNA scaffolds formed by engineered RNA guides of edited ABCA4, where the second internal symmetrical loop (or right bell) is located at varying distances from the mismatch. Guide 0.100.70 (−5, +32) demonstrated an ADAR-mediated RNA editing percentage of more than about 17%, which is the ADAR-mediated RNA editing percentage of guide 0.100.70 without the first and second internal symmetrical loops.

FIG. 12 shows varying guide-target RNA scaffolds formed by engineered RNA guides, where the first internal symmetrical loop (or left bell) is located at varying distances from the mismatch based on guide 0.100.70.

FIG. 13 shows exemplary guide-target RNA scaffolds formed by engineered RNA guides of edited ABCA4, where the first internal symmetrical loop (or left bell) is located at varying distances from the mismatch. Exemplary guides 0.100.70 (−5, +33), 0.100.70 (−6, +33), 0.100.70 (−9, +33), 0.100.70 (−11, +33), 0.100.70 (−12, +33), 0.100.70 (−13, +33), 0.100.70 (−14, +33), and 0.100.70 (−15, +33) demonstrated an ADAR-mediated RNA editing percentage of more than about 34%, which is the ADAR-mediated RNA editing percentage of guide 0.100.70 without the first and second internal symmetrical loops.

FIG. 14 shows the target positions and ADAR-mediated RNA editing percentage for each of the exemplary engineered RNA guides 0.100.70 (−5, +33), 0.100.70 (−9, +33), 0.100.70 (−13, +33), 0.100.70 (−14, +33), and 0.100.70 (−15, +33), and 0.100.70 without the first and second internal symmetrical loops, where the edited base position is identified as the zero target position. Each of the exemplary guides except for 0.100.70 demonstrate an ADAR-mediated RNA editing percentage of more than about 30% at the zero target position or target 0 position.

FIG. 15 shows exemplary guide-target RNA scaffolds formed by engineered RNA guides of edited ABCA4 based on a first internal symmetrical loop (LB) located −9 bases or −15 bases from the mismatch and a second internal symmetrical loop (RB) located +33 bases from the mismatch, where the total length of the guide is varied via 2nt precise deletions from the 5′ end of the guide as well as the length upstream of the mismatch (e.g., 0 position).

FIG. 16 shows exemplary guide-target RNA scaffolds formed by engineered RNA guides of FIG. 15, where the first internal symmetrical loop (or left bell) and the second internal symmetrical loop (or right bell) are maintained at positions −9 and +33, respectively, with varying lengths from the mismatch. Exemplary guides 0.92.62 (−9, +33), 0.94.64 (−9, +33), and 0.96.66 (−9, +33) demonstrated an ADAR-mediated RNA editing percentage of more than about 38%, which is the RNA editing percentage of guide 0.100.70 (−9, +33).

FIG. 17 shows the target positions and ADAR-mediated RNA editing percentage for each of the exemplary engineered RNA guides 0.92.62 (−9, +33), 0.94.64 (−9, +33), and 0.96.66 (−9, +33), where the edited base position is identified as the zero target position. Each of the exemplary guides demonstrate an ADAR-mediated RNA editing percentage of more than about 40% at the zero target position.

FIG. 18 shows exemplary guide-target RNA scaffolds formed by engineered RNA guides of FIG. 15, where the first internal symmetrical loop (or left bell) and the second internal symmetrical loop (or right bell) are maintained at positions −15 and +33, respectively, with varying lengths from the mismatch. Exemplary guides 0.90.60 (−15, +33), 0.92.62 (−15, +33), and 0.94.64 (−15, +33) demonstrated an ADAR-mediated RNA editing percentage of more than about 46%, which is the ADAR-mediated RNA editing percentage of guide 0.96.66 (−15, +33).

FIG. 19 shows the target positions and ADAR-mediated RNA editing percentage for each of the exemplary engineered RNA guides 0.90.60 (−15, +33), 0.92.62 (−15, +33), and 0.94.64 (−15, +33), where the edited base position is identified as the zero target position or target 0 position. Each of the exemplary guides demonstrate an ADAR-mediated RNA editing percentage of more than about 46% at the zero target position or target 0 position.

FIG. 20 shows the target positions and ADAR-mediated RNA editing percentage for exemplary engineered RNA guides pre-improvement (guide 0.100.80 without first and second internal loops) and post-improvement (guide 0.92.62 (−15, +33) with first and second internal loops), where the edited base position is identified as the zero target position. The pre-improvement guide (0.100.80) illustrates an ADAR-mediated RNA editing percentage of about 12% at the zero target position, and the post-improvement guide (0.92.62 (−15, +33)) illustrates an ADAR-mediated RNA editing percentage of about 58% at the zero target position.

FIG. 21 shows exemplary guide-target RNA scaffolds formed by engineered RNA guides, where the target RNAs are ABCA4 with first and second internal loops (−9, +33) and GAPDH with an A-C mismatch (GAPDH 100.70 A-C), with the micro-footprint (GAPDH Shaker mimicry), and GAPDH with first and second internal loops (GAPDH Shaker mimicry −9, +33).

FIG. 22 shows the ADAR-mediated RNA editing percentages of GAPDH engineered RNA guides of FIG. 21 and controls (No transfection; GFP plasmid) for biological replicates. Exemplary GAPDH RNA guides 0.100.70 (−9, +33) demonstrated an ADAR-mediated RNA editing percentage of more than about 25%, which is the ADAR-mediated RNA editing percentage of GAPDH RNA guide 0.100.70 (−9, +33).

FIG. 23 shows the ADAR-mediated RNA editing percentages of GAPDH engineered RNA guides of FIG. 21. Exemplary GAPDH with the A-C mismatch (top panel), with the micro-footprint (middle panel), and GAPDH with first and second internal loops (bottom panel), where the GAPDH engineered RNA guide with the macro-footprint (bottom panel) illustrates an ADAR-mediated RNA editing percentage of more than about 20% at the zero target position.

FIG. 24 shows exemplary guide-target RNA scaffolds formed by engineered RNA guides, where the target RNA is Rab7a with an A-C mismatch (Rab7a 100.70 A-C; SEQ ID NO:109), with the micro-footprint (Rab7a 100.70 Shaker mimicry; SEQ ID NO:110), and Rab7a with first and second internal loops (Rab7a Shaker mimicry −9,+33; SEQ ID NO111).

FIG. 25 shows the ADAR-mediated RNA editing percentages of Rab7a engineered RNA guides of FIG. 24 and controls (No transfection; GFP plasmid) for biological replicates. Exemplary Rab7a RNA guide 0.100.70 (−9, +33) demonstrated an ADAR-mediated RNA editing percentage of about 30%, where the controls demonstrated an ADAR-mediated RNA editing percentage of about 5%.

FIG. 26 shows the target positions and ADAR-mediated RNA editing percentages of Rab7a engineered RNA guides of FIG. 24, where the edited base position is identified as the zero target position or target 0 position. The Rab7a shaker mimicry guide (0.100.70 (−9, +33)) illustrates an ADAR-mediated RNA editing percentage of more than about 20% at the zero target position with fewer off-target edits.

FIG. 27 shows exemplary guide-target RNA scaffolds formed by engineered RNA guides, where the target RNA is APP with an A-C mismatch (APP 100.70 A-C; SEQ ID NO:112), with the micro-footprint (APP 100.70 Shaker mimicry; SEQ ID NO:113), and APP with first and second internal loops (APP Shaker mimicry −9,+33; SEQ ID NO:114).

FIG. 28 shows the ADAR-mediated RNA editing percentages of APP engineered RNA guides of FIG. 27 and controls (No transfection; GFP plasmid) for biological replicates. Exemplary APP RNA guide 0.100.70 (−9, +33) demonstrated an ADAR-mediated RNA editing percentage of about 13%, where the controls demonstrated an ADAR-mediated RNA editing percentage of about 2%.

FIG. 29 shows the target positions and ADAR-mediated RNA editing percentages of APP engineered RNA guides of FIG. 27, where the edited base position is identified as the zero target position or target 0 position. The APP shaker mimicry guide (0.100.70 (−9, +33)) illustrates an ADAR-mediated RNA editing percentage of more than about 20% at the zero target position with fewer off-target edits than the A-C mismatch or Shaker mimicry MAPT RNA guides.

FIG. 30 shows exemplary guide-target RNA scaffolds formed by engineered RNA guides, where the target RNA is MAPT with an A-C mismatch (MAPT 100.70 A-C, with the micro-footprint (MAPT 100.70 Shaker mimicry), and MAPT with first and second internal loops (MAPT Shaker mimicry −9,+33).

FIG. 31 shows the ADAR-mediated RNA editing percentages of MAPT engineered RNA guides of FIG. 30 and controls (No transfection; GFP plasmid) for biological replicates. Exemplary MAPT RNA guide 0.100.70 (−9, +33) demonstrated an ADAR-mediated RNA editing percentage of more than about 40%, where the controls demonstrated an ADAR-mediated RNA editing percentage of less than about 5%.

FIG. 32 shows the target positions and ADAR-mediated RNA editing percentages of MAPT engineered RNA guides of FIG. 30, where the edited base position is identified as the zero target position or target 0 position. The MAPT shaker mimicry guide (0.100.70 (−9, +33)) illustrates an ADAR-mediated RNA editing percentage of more than about 40% at the zero target position with fewer off-target edits than the A-C mismatch or Shaker mimicry MAPT RNA guides.

FIG. 33 shows a summary of how a library for screening longer self-annealing RNA structures was generated.

FIG. 34 shows a comparison of cell-free RNA editing using the high throughput described here versus in-cell RNA editing facilitated via the same engineered guide RNA sequence at various timepoints.

FIG. 35 shows heatmaps of all self-annealing RNA structures tested for 4 micro-footprints (A/C mismatch, 2108, 871, and 919) formed within varying placement of a barbell macro-footprint. The y-axis shows all candidate engineered guide RNAs tested and the x-axis shows the target sequence positions, with position 0 representing the target adenosine to be edited.

FIG. 36 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side).

FIG. 37 depicts on-target editing (x-axis) vs. specificity (y-axis) of various guide RNAs via ADAR against LRRK2 (left most), APP/GRN/SNCA (top row, left to right), and DUX4/ABCA4/SERPINA1 (bottom row, left to right).

FIGS. 38A-D show LRRK2 RNA editing profiles of various engineered guide RNAs of the present disclosure via ADAR.

FIG. 39 shows the LRRK2 ADAR-mediated RNA editing profile of an engineered guide RNA of the present disclosure, which forms a barbell macro-footprint and a micro-footprint in the guide-target RNA scaffold.

FIG. 40 depicts an illustration of a strategy to minimize +1 editing by modulating structures within the guide-target RNA complex.

FIG. 41 depicts tiling of symmetrical internal loops within the guide-target RNA complex to minimize +1 editing.

FIG. 42 depicts engineering of guides that edit the target adenosine of ABCA4 with minimal +1 editing by utilizing symmetrical internal loops within the guide-target RNA complex.

FIG. 43 is a summary of showing the progression of engineering of a candidate guide RNA to minimize +1 editing by utilizing symmetrical internal loops within the guide-target RNA complex.

FIGS. 44A-44C depict the ADAR-mediated RNA editing efficiency of guide RNAs designed through machine learning targeting LRRK2 in an in-cell editing model, each having a barbell macro-footprint with symmetrical internal loops at positions −20 and +26.

FIG. 45 shows LRRK2 target RNA editing for a control engineered guide and exemplary engineered guide 919 via ADAR1 and ADAR1+ADAR2.

FIG. 46 shows LRRK2 target RNA editing for exemplary engineered guide 1976 and exemplary engineered guide 2397 via ADAR1 and ADAR1+ADAR2.

FIG. 47 shows LRRK2 target RNA editing for exemplary engineered guide 871 and exemplary engineered guide 610 via ADAR1 and ADAR1+ADAR2.

FIG. 48 shows LRRK2 target RNA editing for exemplary engineered guides ML generative 0703 and ML generative 0719 designed by machine learning via ADAR1 and ADAR1+ADAR2.

FIG. 49 shows LRRK2 target RNA editing for exemplary engineered guides ML generative 0728 and ML generative 0732 designed by machine learning via ADAR1 and ADAR1+ADAR2.

FIG. 50 shows LRRK2 target RNA editing for exemplary engineered guides ML generative 0733 and ML generative 0742 designed by machine learning via ADAR1 and ADAR1+ADAR2.

FIG. 51 shows LRRK2 target RNA editing for exemplary engineered guides ML generative 0743 and ML generative 0745 designed by machine learning via ADAR1 and ADAR1+ADAR2.

FIG. 52 shows LRRK2 target RNA editing for exemplary engineered guides ML generative 0766 and ML generative 0769 designed by machine learning via ADAR1 and ADAR1+ADAR2.

FIG. 53 shows LRRK2 target RNA editing for exemplary engineered guides ML generative 0766 and ML generative 0769 designed by machine learning via ADAR1 and ADAR1+ADAR2.

FIG. 54 shows LRRK2 target RNA editing for exemplary engineered guides ML exhaustive 0049 and ML exhaustive 0069 designed by machine learning via ADAR1 and ADAR1+ADAR2.

FIG. 55 shows LRRK2 target RNA editing for exemplary engineered guides ML exhaustive 0090 and ML exhaustive 0139 designed by machine learning via ADAR1 and ADAR1+ADAR2.

FIG. 56 shows LRRK2 target RNA editing for exemplary engineered guides ML generative 0274 and ML generative 0325 designed by machine learning via ADAR1 and ADAR1+ADAR2.

FIG. 57 shows LRRK2 target RNA editing for exemplary engineered guides ML generative 0332 and ML generative 0559 designed by machine learning via ADAR1 and ADAR1+ADAR2.

FIG. 58 shows LRRK2 target RNA editing for exemplary engineered guides ML generative 0639 and ML generative 0643 designed by machine learning via ADAR1 and ADAR1+ADAR2.

FIG. 59 shows LRRK2 target RNA editing for exemplary engineered guides ML generative 0644 and ML generative 0690 designed by machine learning via ADAR1 and ADAR1+ADAR2.

FIG. 60 shows LRRK2 target RNA editing for exemplary engineered guides ML generative 0699 and ML generative 0701 designed by machine learning via ADAR1 and ADAR1+ADAR2.

FIG. 61 shows LRRK2 target RNA editing for exemplary engineered guides ML exhaustive 0395 and ML exhaustive 0453 designed by machine learning via ADAR1 and ADAR1+ADAR2.

FIG. 62 shows LRRK2 target RNA editing for exemplary engineered guides ML exhaustive 0464 and ML exhaustive 1042 designed by machine learning via ADAR1 and ADAR1+ADAR2.

FIG. 63 shows LRRK2 target RNA editing for exemplary engineered guides ML generative 0002 and ML generative 0013 designed by machine learning via ADAR1 and ADAR1+ADAR2.

FIG. 64 shows LRRK2 target RNA editing for exemplary engineered guides ML generative 0016 and ML generative 0043 designed by machine learning via ADAR1 and ADAR1+ADAR2.

FIG. 65 shows LRRK2 target RNA editing for exemplary engineered guides ML generative 0058 and ML generative 0071 designed by machine learning via ADAR1 and ADAR1+ADAR2.

FIG. 66 shows LRRK2 target RNA editing for exemplary engineered guides ML generative 0130 and ML generative 0156 designed by machine learning via ADAR1 and ADAR1+ADAR2.

FIG. 67 shows LRRK2 target RNA editing for exemplary engineered guides ML generative 0176 and ML generative 0218 designed by machine learning via ADAR1 and ADAR1+ADAR2.

FIG. 68 shows LRRK2 target RNA editing for exemplary engineered guides ML exhaustive 1045 and ML exhaustive 1540 designed by machine learning via ADAR1 and ADAR1+ADAR2.

FIG. 69 shows LRRK2 target RNA editing for exemplary engineered guides ML exhaustive 0315 and ML exhaustive 0414 designed by machine learning via ADAR1 and ADAR1+ADAR2.

FIG. 70 shows LRRK2 target RNA editing for exemplary engineered guide ML exhaustive 0013 designed by machine learning via ADAR1 and ADAR1+ADAR2.

FIG. 71A-71B depict selection of two exemplary LRRK2 guide RNAs designed through machine learning for further engineering.

FIG. 72 shows a plot of editing specificity of LRRK2 exhaustive guide RNAs designed through machine learning via ADAR1, ADAR2, or ADAR1+ADAR2.

FIG. 73 shows exemplary LRRK2 exhaustive guide RNAs designed through machine learning that display specificity for ADAR2.

FIGS. 74A and 74B show the top performing guide RNAs that display specificity for ADAR1+ADAR2.

FIGS. 75A and 75B show the top performing guide RNAs that display specificity for ADAR2.

FIGS. 76A and 76B show the top performing guide RNAs that display specificity for ADAR1.

FIG. 77 depicts a comparison between ML-derived gRNAs and gRNAs generated using in vitro high throughput screening (HTS) methods.

FIG. 78 depicts an overview of the engineering of guide RNAs produced from high-throughput screening.

FIGS. 79A and 79B depict cell-free and in-cell editing of exemplary LRRK2 guide610 without a barbell macro-footprint (FIG. 79A) and with a barbell macro-footprint (FIG. 79B) via ADAR.

FIGS. 80A-80C show engineering of the macro-footprint position for an exemplary guide610 targeting LRRK2. FIG. 80A shows tiling of the macro-footprint positioning for the exemplary guide with respect to the A/C mismatch, and how this tiling affects editing via ADAR1 and ADAR1+ADAR2. FIG. 80B shows the percent editing for the guide variants via ADAR1. FIG. 80C shows the percent editing for the guide variants via ADAR1+ADAR2.

FIGS. 81A-81C show engineering of right barbell coordinates for an exemplary guide610 targeting LRRK2. As shown in FIG. 81A, the coordinate of the right barbell was tiled between the following coordinates with respect to the A/C mismatch: +22. +23, +24, +25, +26, +28, +30, +32, and +34, and the effect of each position on ADAR1 and ADAR1+ADAR2 editing was determined. FIG. 81B shows the percent editing for the exemplary guide variants via ADAR1. FIG. 81C shows the percent editing for the exemplary guide variants via ADAR1+ADAR2.

FIGS. 82A and 82B show engineering of left barbell coordinates for an exemplary guide targeting LRRK2. As shown in FIG. 82A, the coordinate of the left barbell was tiled between the following coordinates with respect to the A/C mismatch: −10, −12, −14, −16, −18, −20, −22, and −24, and the effect of each position on ADAR1 and ADAR1+ADAR2 editing was determined. FIG. 82B shows the percent editing for the exemplary guide variants via ADAR1.

FIGS. 83A and 83B show engineering of guide length for an exemplary guide targeting LRRK2. FIG. 83A depicts the effect of guide length on ADAR1 and ADAR1+ADAR2 editing.

FIG. 83B shows the percent editing for the exemplary guide variants of varying length via ADAR1. The y-axis shows all candidate engineered guide RNAs tested and the x-axis shows the target sequence positions, with position 0 representing the target adenosine to be edited.

FIGS. 84A and 84B show in cell and cell-free editing of LRRK2 by exemplary guide RNA 2063 variants without a barbell (FIG. 84A) and having a barbell (FIG. 84B) via ADAR1 and ADAR1+ADAR2. The y-axis shows all candidate engineered guide RNAs tested and the x-axis shows the target sequence positions, with position 0 representing the target adenosine to be edited.

FIGS. 85A and 85B show in cell and cell-free editing of LRRK2 by exemplary guide RNA 1590 variants without a barbell (FIG. 85A) and having a barbell (FIG. 85B) via ADAR1 and ADAR1+ADAR2. The y-axis shows all candidate engineered guide RNAs tested and the x-axis shows the target sequence positions, with position 0 representing the target adenosine to be edited.

FIGS. 86A-86C show in cell and cell-free editing of LRRK2 by exemplary guide RNA 2397 variants without a barbell (FIG. 86A) and having a barbell (FIG. 86B and FIG. 86C) via ADAR1 and ADAR1+ADAR2. The y-axis shows all candidate engineered guide RNAs tested and the x-axis shows the target sequence positions, with position 0 representing the target adenosine to be edited.

FIGS. 87A-87C show engineering of the macro-footprint positioning for exemplary guide 2397 RNA variants. FIG. 87A depicts a summary of the RNA editing efficiencies for the exemplary guide 2397 RNA variants, while FIG. 87B and FIG. 87C depict the editing efficiency by position for each exemplary guide RNA via ADAR1 (FIG. 87B) and ADAR1+ADAR2 (FIG. 87C). The y-axis shows all candidate engineered guide RNAs tested and the x-axis shows the target sequence positions, with position 0 representing the target adenosine to be edited.

FIGS. 88A-88C show engineering of the right barbell coordinate for exemplary guide 2397 RNA variants. FIG. 88A depicts a summary of the RNA editing efficiencies for the exemplary guide 2397 RNA variants, while FIG. 88B and FIG. 88C depict the editing efficiency by position for each exemplary guide RNA via ADAR1 (FIG. 88B) and ADAR1+ADAR2 (FIG. 88C). The y-axis shows all candidate engineered guide RNAs tested and the x-axis shows the target sequence positions, with position 0 representing the target adenosine to be edited.

FIG. 89 depicts engineering of the left barbell coordinate for exemplary guide 2397 RNA variants.

FIGS. 90A and 90B show in cell and cell-free editing of LRRK2 by exemplary guide RNA 1321 variants without a barbell (FIG. 90A) and having a barbell (FIG. 90B) via ADAR1 and ADAR1+ADAR2. The y-axis shows all candidate engineered guide RNAs tested and the x-axis shows the target sequence positions, with position 0 representing the target adenosine to be edited.

FIGS. 91A and 91B show in cell and cell-free editing of LRRK2 by exemplary guide RNA 295 variants without a barbell (FIG. 91A) and having a barbell (FIG. 91B) via ADAR1 and ADAR1+ADAR2. The y-axis shows all candidate engineered guide RNAs tested and the x-axis shows the target sequence positions, with position 0 representing the target adenosine to be edited.

FIGS. 92A and 92B show in cell and cell-free editing of LRRK2 by exemplary guide RNA 730 variants without a barbell (FIG. 92A) and having a barbell (FIG. 92B) via ADAR1 and ADAR1+ADAR2. The y-axis shows all candidate engineered guide RNAs tested and the x-axis shows the target sequence positions, with position 0 representing the target adenosine to be edited.

FIGS. 93A and 93B show in cell and cell-free editing of LRRK2 by exemplary guide RNA 708 variants without a barbell (FIG. 93A) and having a barbell (FIG. 93B) via ADAR1 and ADAR1+ADAR2. The y-axis shows all candidate engineered guide RNAs tested and the x-axis shows the target sequence positions, with position 0 representing the target adenosine to be edited.

FIGS. 94A and 94B show in cell and cell-free editing of LRRK2 by exemplary guide RNA 351 variants without a barbell (FIG. 94A) and having a barbell (FIG. 94B) via ADAR1 and ADAR1+ADAR2. The y-axis shows all candidate engineered guide RNAs tested and the x-axis shows the target sequence positions, with position 0 representing the target adenosine to be edited.

FIGS. 95A and 95B show in cell and cell-free editing of LRRK2 by exemplary guide RNA 1326 variants without a barbell (FIG. 95A) and having a barbell (FIG. 95B) via ADAR1 and ADAR1+ADAR2. The y-axis shows all candidate engineered guide RNAs tested and the x-axis shows the target sequence positions, with position 0 representing the target adenosine to be edited.

FIGS. 96A-960 show in cell and cell-free editing of LRRK2 by exemplary guide RNA 871 variants without a barbell (FIG. 96A) and having barbells (FIG. 96B-FIG. 960) via ADAR1 and ADAR1+ADAR2. The y-axis shows all candidate engineered guide RNAs tested and the x-axis shows the target sequence positions, with position 0 representing the target adenosine to be edited.

FIGS. 97A-97C show engineering of the macro-footprint positioning for exemplary guide 871 RNA variants. FIG. 97A depicts a summary of the RNA editing efficiencies for the exemplary guide 871 RNA variants, while FIG. 97B and FIG. 97C depict the editing efficiency by position for each exemplary guide RNA via ADAR1 (FIG. 97B) and ADAR1+ADAR2 (FIG. 97C). The y-axis shows all candidate engineered guide RNAs tested and the x-axis shows the target sequence positions, with position 0 representing the target adenosine to be edited.

FIGS. 98A-98C show engineering of the right barbell coordinate for exemplary guide 871 RNA variants. FIG. 98A depicts a summary of the RNA editing efficiencies for the exemplary guide 871 RNA variants, while FIG. 98B and FIG. 98C depict the editing efficiency by position for each exemplary guide RNA via ADAR1 (FIG. 98B) and ADAR1+ADAR2 (FIG. 98C). The y-axis shows all candidate engineered guide RNAs tested and the x-axis shows the target sequence positions, with position 0 representing the target adenosine to be edited.

FIGS. 99A-99C show engineering of the left barbell coordinate for exemplary guide 871 RNA variants. FIG. 99A depicts a summary of the RNA editing efficiencies for the exemplary guide 871 RNA variants, while FIG. 99B and FIG. 99C depict the editing efficiency by position for each exemplary guide RNA via ADAR1 (FIG. 99B) and ADAR1+ADAR2 (FIG. 99C). The y-axis shows all candidate engineered guide RNAs tested and the x-axis shows the target sequence positions, with position 0 representing the target adenosine to be edited.

FIGS. 100A-100C show engineering of the guide length for exemplary guide 871 RNA variants. FIG. 100A depicts a summary of the RNA editing efficiencies for the exemplary guide 871 RNA variants, while FIG. 100B and FIG. 100C depict the editing efficiency by position for each exemplary guide RNA via ADAR1 (FIG. 100B) and ADAR1+ADAR2 (FIG. 100C). The y-axis shows all candidate engineered guide RNAs tested and the x-axis shows the target sequence positions, with position 0 representing the target adenosine to be edited.

FIGS. 101A-11T show in cell and cell-free editing of LRRK2 by exemplary guide RNA 919 variants. FIG. 101A provides a summary of the in cell editing data for the exemplary guide 919 variants via ADAR1 and ADAR1+ADAR2. FIG. 101B-FIG. 101T depict the editing efficiency by position for each exemplary guide 919 RNA via ADAR1 and ADAR1+ADAR2. The y-axis shows all candidate engineered guide RNAs tested and the x-axis shows the target sequence positions, with position 0 representing the target adenosine to be edited.

FIGS. 102A-102C show engineering of the macro-footprint positioning for exemplary guide 919 RNA variants. FIG. 102A depicts a summary of the RNA editing efficiencies for the exemplary guide 919 RNA variants, while FIG. 102B and FIG. 102C depict the editing efficiency by position for each exemplary guide RNA via ADAR1 (FIG. 102B) and ADAR1+ADAR2 (FIG. 102C). The y-axis shows all candidate engineered guide RNAs tested and the x-axis shows the target sequence positions, with position 0 representing the target adenosine to be edited.

FIGS. 103A-103C show engineering of the right barbell coordinate for exemplary guide 919 RNA variants. FIG. 103A depicts a summary of the RNA editing efficiencies for the exemplary guide 919 RNA variants, while FIG. 103B and FIG. 103C depict the editing efficiency by position for each exemplary guide RNA via ADAR1 (FIG. 103B) and ADAR1+ADAR2 (FIG. 103C). The y-axis shows all candidate engineered guide RNAs tested and the x-axis shows the target sequence positions, with position 0 representing the target adenosine to be edited.

FIGS. 104A-104C show engineering of the left barbell coordinate for exemplary guide 919 RNA variants. FIG. 104A depicts a summary of the RNA editing efficiencies for the exemplary guide 919 RNA variants, while FIG. 104B and FIG. 104C depict the editing efficiency by position for each exemplary guide RNA via ADAR1 (FIG. 104B) and ADAR1+ADAR2 (FIG. 104C). The y-axis shows all candidate engineered guide RNAs tested and the x-axis shows the target sequence positions, with position 0 representing the target adenosine to be edited.

FIGS. 105A-105C show engineering of the guide length for exemplary guide 919 RNA variants. FIG. 105A depicts a summary of the RNA editing efficiencies for the exemplary guide 919 RNA variants, while FIG. 105B and FIG. 105C depict the editing efficiency by position for each exemplary guide RNA via ADAR1 (FIG. 105B) and ADAR1+ADAR2 (FIG. 105C). The y-axis shows all candidate engineered guide RNAs tested and the x-axis shows the target sequence positions, with position 0 representing the target adenosine to be edited.

FIGS. 106A-106C show in cell and cell-free editing of LRRK2 by exemplary guide RNA 844 variants via ADAR1 and ADAR1+ADAR2. The y-axis shows all candidate engineered guide RNAs tested and the x-axis shows the target sequence positions, with position 0 representing the target adenosine to be edited.

FIGS. 107A-107C show in cell and cell-free editing of LRRK2 by exemplary guide RNA 1976 variants via ADAR1 and ADAR1+ADAR2. The y-axis shows all candidate engineered guide RNAs tested and the x-axis shows the target sequence positions, with position 0 representing the target adenosine to be edited.

FIGS. 108A-108C show engineering of the macro-footprint positioning for exemplary guide 1976 RNA variants. FIG. 108A depicts a summary of the RNA editing efficiencies for the exemplary guide 1976 RNA variants, while FIG. 108B and FIG. 108C depict the editing efficiency by position for each exemplary guide RNA via ADAR1 (FIG. 108B) and ADAR1+ADAR2 (FIG. 108C). The y-axis shows all candidate engineered guide RNAs tested and the x-axis shows the target sequence positions, with position 0 representing the target adenosine to be edited.

FIGS. 109A-109C show engineering of the right barbell coordinate for exemplary guide 1976 RNA variants. FIG. 109A depicts a summary of the RNA editing efficiencies for the exemplary guide 1976 RNA variants, while FIG. 109B and FIG. 109C depict the editing efficiency by position for each exemplary guide RNA via ADAR1 (FIG. 109B) and ADAR1+ADAR2 (FIG. 109C). The y-axis shows all candidate engineered guide RNAs tested and the x-axis shows the target sequence positions, with position 0 representing the target adenosine to be edited.

FIG. 110 depicts engineering of the left barbell coordinate for exemplary guide 1976 RNA variants.

FIG. 111 shows in cell and cell-free editing of LRRK2 by an exemplary guide RNA 1700. The y-axis shows all candidate engineered guide RNAs tested and the x-axis shows the target sequence positions, with position 0 representing the target adenosine to be edited.

FIGS. 112A-112E show in cell and cell-free editing of LRRK2 by exemplary guide RNA 860 variants. The y-axis shows all candidate engineered guide RNAs tested and the x-axis shows the target sequence positions, with position 0 representing the target adenosine to be edited.

FIG. 113 shows in cell and cell-free editing of LRRK2 by an exemplary guide RNA 2108. The y-axis shows all candidate engineered guide RNAs tested and the x-axis shows the target sequence positions, with position 0 representing the target adenosine to be edited.

FIG. 114 depicts a comparison of editing efficiency between exemplary guide RNA variants targeting LRRK2.

FIG. 115 depicts an scAAV vector map for in vitro screening of LRRK2 guide RNA variant produced herein when expressed in an AAV vector.

FIGS. 116A and 116B depict editing efficiencies of exemplary LRRK2 guide provided herein when transfected as an scAAV vector plasmid (FIG. 116A) or transduced as an scAAVDJ virus (FIG. 116B) via ADAR.

FIGS. 117A and 117B depict editing of ABCA4 mRNA using exemplary guide RNAs as described here via ADAR.

FIG. 118 illustrates an exemplary guide RNA capable of facilitating ADAR-mediated editing of a target adenosine in an ABCA4 mRNA having a 5′G next to the target adenosine.

FIG. 119 depicts tiling of the position of the barbell macro-footprint along the guide-target RNA scaffold to identify those engineered guide RNAs that facilitate the highest level of SERPINA1 editing.

FIG. 120 shows design of the right and left barbell coordinates, as well as engineering of guide length for the exemplary engineered guide 06566 RNA.

FIG. 121 depicts in cell ADAR-mediated editing of exemplary engineered guide RNAs targeting SERPINA1 using a GFP editing reporting screen. The y-axis shows all candidate engineered guide RNAs tested and the x-axis shows the target sequence positions, with position 0 representing the target adenosine to be edited.

FIG. 122 depicts the ADAR-mediated editing efficiency for an exemplary engineered guide SERP-AC-AA_95-50_-10_25 targeting SERPINA1.

FIG. 123 depicts the ADAR-mediated editing efficiency for an exemplary engineered guide SERP-AC-AA_95-50_-8_28 targeting SERPINA1.

FIG. 124 depicts the ADAR-mediated editing efficiency for an exemplary engineered guide SERP-AC-AA_95-50_-10_26 targeting SERPINA1.

FIG. 125 depicts the ADAR-mediated editing efficiency for an exemplary engineered guide SERP-100.50-position_-20 adenosine scan control targeting SERPINA1.

FIG. 126 depicts the ADAR1-mediated editing efficiency for an AAV vector encoding an exemplary engineered guide targeting ABCA4 G1961E in human cells.

FIG. 127 depicts a workflow for screening exemplary guide RNAs targeting LRRK2 in a broken GFP reporter system.

FIG. 128 depicts the editing efficiency of the exemplary guides targeting LRRK2 in the broken GFP reporter system via exogenous or endogenous ADAR.

FIG. 129 shows the change in body weight for subjects administered an AAV vector encoding an exemplary engineered guide RNA targeting SERPINA1 and control vector over the course of the 28 day study.

FIG. 130 illustrates transduction of an engineered guide RNA payload in the liver after administration of an AAV vector encoding an exemplary engineered guide RNA targeting SERPINA1 and control vector, as measured by expression of an mCherry reporter.

FIG. 131 depicts normalized quantitation of an engineered guide RNA after administration of an AAV vector encoding an exemplary engineered guide RNA targeting SERPINA1 and control vector.

FIG. 132 depicts quantitation of the amount of target adenosine editing of a SERPINA1 RNA via ADAR through administration of an AAV vector encoding an engineered guide RNA targeting SERPINA1, as compared to the level of editing of the control AAV vector.

FIG. 133 provides a comparison between linear and circularized versions of exemplary guide RNAs targeting LRRK2.

FIG. 134A-FIG. 134B depict engineering of the length of circularized LRRK2 guide RNAs by increasing the length of the circularized guide RNA by an additional 15 nucleotides (FIG. 134A), 30 nucleotides (FIG. 134A), and 100 nucleotides (FIG. 134B).

FIG. 135 depicts the effect of deletion of selected uridines from an engineered circularized guide RNA targeting LRRK2 on editing of a target LRRK2 RNA.

FIG. 136A-FIG. 136D illustrate the in vivo editing of a target LRRK2 RNA upon administration of an scAAV vector encoding an engineered guide RNA targeting LRRK2. FIG. 136A and FIG. 136C depict the in vivo editing efficiencies for the scAAV vector encoding the engineered guide RNA targeting LRRK2, as measured in the brain (FIG. 136A) and liver (FIG. 136C). FIG. 136B and FIG. 136D illustrate quantitation of engineered guide RNA expression, as compared to expression of the GAPDH control, in the brain (FIG. 136B) and liver (FIG. 136D).

FIG. 137 shows results of a library screen of SERPINA1 targeting guides. The ADAR1 fraction edited is depicted on the Y-axis and the specificity score is depicted on the X-axis.

### DETAILED DESCRIPTION

Engineered Guides with Barbell Macro-Footprints

Disclosed herein are engineered guide RNAs for site-specific editing of an adenosine of a target RNA via an adenosine deaminase enzyme. Engineered guide RNAs of the present disclosure comprise a micro-footprint sequence and a barbell macro-footprint sequence that each comprise latent structures, such that when the engineered guide RNA is hybridized to the target RNA, the latent structures manifest. A latent structure, when manifested, produces at least one structural feature selected from the group consisting of: a bulge, an internal loop, a mismatch, a hairpin, and any combination thereof. In some embodiments, the engineered guide RNA of the disclosure, upon hybridization of the engineered guide RNA and the sequence of the target RNA form a guide-target RNA scaffold, comprising (i) a region that comprises at least one structural feature; and (ii) a first internal loop (also referred to as a “left bell” or “LB”) and a second internal loop (also referred to as a “right bell” or “RB”) that flank opposing ends of the region of the guide-target RNA scaffold, where the engineered guide RNA facilitates an increase in the amount of the targeted edit of the adenosine of the target RNA via the adenosine deaminase enzyme RNA editing entity, relative to an otherwise comparable engineered guide RNA lacking the first internal loop and the second internal loop.

As described herein, a “micro-footprint” sequence refers to a sequence with latent structures that, when manifested, facilitate editing of the adenosine of a target RNA via an adenosine deaminase enzyme. A macro-footprint can serve to guide an RNA editing entity (e.g., ADAR) and direct its activity towards a micro-footprint. In some embodiments, included within the micro-footprint sequence is a nucleotide that is positioned such that, when the guide RNA is hybridized to the target RNA, the nucleotide opposes the adenosine to be edited by the adenosine deaminase and does not base pair with the adenosine to be edited. This nucleotide is referred to herein as the “mismatched position” or “mismatch” and can be a cytosine. Micro-footprint sequences as described herein have upon hybridization of the engineered guide RNA and target RNA, at least one structural feature selected from the group consisting of: a bulge, an internal loop, a mismatch, a hairpin, and any combination thereof. Engineered guide RNAs with superior micro-footprint sequences can be selected based on their ability to facilitate editing of a specific target RNA. Engineered guide RNAs selected for their ability to facilitate editing of a specific target are capable of adopting various micro-footprint latent structures, which can vary on a target-by-target basis.

Guide RNAs of the present disclosure further comprise a macro-footprint. In some embodiments, the macro-footprint comprises a barbell macro-footprint. A micro-footprint can serve to guide an RNA editing enzyme and direct its activity towards the target adenosine to be edited. A “barbell” as described herein refers to a pair of internal loop latent structures that manifest upon hybridization of the guide RNA to the target RNA. In some embodiments, each internal loop is positioned towards the 5′ end or the 3′ end of the guide-target RNA scaffold formed upon hybridization of the guide RNA and the target RNA. In some embodiments, each internal loop flanks opposing sides of the micro-footprint sequence. Insertion of a barbell macro-footprint sequence flanking opposing sides of the micro-footprint sequence, upon hybridization of the guide RNA to the target RNA, results in formation of barbell internal loops on opposing sides of the micro-footprint, which in turn comprises at least one structural feature that facilitates editing of a specific target RNA.

The present disclosure demonstrates that the presence of barbells flanking the micro-footprint can improve one or more aspects of editing. For example, the presence of a barbell macro-footprint in addition to a micro-footprint can result in a higher amount of on target adenosine editing, relative to an otherwise comparable guide RNA lacking the barbells. Additionally, and or alternatively, the presence of a barbell macro-footprint in addition to a micro-footprint can result in a lower amount of local off-target adenosine editing, relative to an otherwise comparable guide RNA lacking the barbells. Further, while the effect of various micro-footprint structural features can vary on a target-by-target basis based on selection in a high throughput screen, the present disclosure demonstrates that the increase in the one or more aspects of editing provided by the barbell macro-footprint structures is independent of the particular target RNA. Thus, the present disclosure provides a facile method of improving editing of guide RNAs previously selected to facilitate editing of a target RNA of interest. For example, the barbell macro-footprint and the micro-footprint of the disclosure can provide an increased amount of on target adenosine editing relative to an otherwise comparable guide RNA lacking the barbells. In other embodiments, the presence of the barbell macro-footprint in addition to the micro-footprint described here can result in a lower amount of local off-target adenosine editing, relative to an otherwise comparable guide RNA, upon hybridization of the guide RNA and target RNA to form a guide-target RNA scaffold lacking the barbells.

A macro-footprint of the present disclosure, such as a barbell macro-footprint, can be comprised in an engineered guide RNA designed to target any number of target RNAs. Target RNAs encompassed by the present disclosure include, but are not limited to, ABCA4, APP, CFTR, DMPK, DUX4, GAPDH, GBA, GRN, HEXA, LIPA, LRRK2, MAPT, PINK1, PMP22, RAB7A, SERPINA1, SNCA, SOD1, a fragment of any one of these, or any combination thereof.

### Engineered Guide RNAS

Provided herein are engineered guide RNAs (and engineered polynucleotides encoding an engineered guide RNA of the present disclosure) comprising a micro-footprint sequence and a barbell macro-footprint sequence for site-specific editing of a target RNA via an adenosine deaminase enzyme. As used herein, the term “engineered” in reference to a guide RNA or polynucleotide encoding the same refers to a non-naturally occurring guide RNA or polynucleotide encoding the same. An engineered guide RNA as disclosed herein comprises a targeting domain with complementarity to a target RNA, where hybridization of the targeting domain of the engineered guide RNA to the target RNA produces latent structures in a guide-target RNA scaffold that manifest upon hybridization into structural features as described herein. A “guide-target RNA scaffold” as described herein can also be referred to as a “double stranded RNA substrate.” As disclosed herein, inclusion of a barbell macro-footprint sequence in the targeting domain to produce a pair of internal loop structural features flanking the micro-footprint sequence improves one or more aspects of editing, as compared to an otherwise comparable guide RNA lacking the barbell macro-footprint sequence (and hence the pair of internal loop structural features).

### Barbell Macro-Footprints

Disclosed herein are barbell macro-footprint sequences that, upon hybridization to a target RNA, produce a pair of internal loop structural features, where the presence of the internal loop structural features improves one or more aspects of editing, as compared to an otherwise comparable guide RNA lacking the pair of internal loop structural features. In some instances, inclusion of a barbell macro-footprint sequence improves an amount of editing of an adenosine of interest (e.g., an on-target adenosine), relative to an amount of editing of on-target adenosine in a comparable guide RNA lacking the barbell macro-footprint sequence. In some instances, inclusion of a barbell macro-footprint sequence decreases an amount of editing of adenosines other than the adenosine of interest (e.g., decreases off-target adenosine), relative to an amount of off-target adenosine in a comparable guide RNA lacking the barbell macro-footprint sequence.

As disclosed herein, a “macro-footprint” sequence can be positioned such that it flanks a micro-footprint sequence. Further, while a macro-footprint sequence can flank a micro-footprint sequence, additional latent structures can be incorporated that flank either end of the macro-footprint as well. In some embodiments, such additional latent structures are included as part of the macro-footprint. In some embodiments, such additional latent structures are separate, distinct, or both separate and distinct from the macro-footprint.

In some embodiments, a macro-footprint sequence can comprise a barbell macro-footprint sequence comprising latent structures that, when manifested, produce a first internal loop and a second internal loop.

In some examples, a first internal loop is positioned “near the 5′ end of the guide-target RNA scaffold” and a second internal loop is positioned near the 3′ end of the guide-target RNA scaffold. The length of the dsRNA comprises a 5′ end and a 3′ end, where up to half of the length of the guide-target RNA scaffold at the 5′ end can be considered to be “near the 5′ end” while up to half of the length of the guide-target RNA scaffold at the 3′ end can be considered “near the 3′ end.” Non-limiting examples of the 5′ end can include about 50% or less of the total length of the dsRNA at the 5′ end, about 45%, about 40%, about 35%, about 30%, about 25%, about 20%, about 15%, about 10%, or about 5%. Non-limiting examples of the 3′ end can include about 50% or less of the total length of the dsRNA at the 3′ end about 45%, about 40%, about 35%, about 30%, about 25%, about 20%, about 15%, about 10%, or about 5%.

The engineered guide RNAs of the disclosure comprising a barbell macro-footprint sequence (that manifests as a first internal loop and a second internal loop) can improve RNA editing efficiency, increase the amount or percentage of RNA editing generally, as well as for on-target nucleotide editing, such as on-target adenosine. In some embodiments, the engineered guide RNAs of the disclosure comprising a first internal loop and a second internal loop can also facilitate a decrease in the amount of or reduce off-target nucleotide editing, such as off-target adenosine or unintended adenosine editing. The decrease or reduction in some examples can be of the number of off-target edits or the percentage of off-target edits.

Each of the first and second internal loops of the barbell macro-footprint can independently be symmetrical or asymmetrical, where symmetry is determined by the number of bases or nucleotides of the engineered guide RNA and the number of bases or nucleotides of the target RNA, that together form each of the first and second internal loops.

As described herein, a double stranded RNA (dsRNA) substrate (e.g., a guide-target RNA scaffold) is formed upon hybridization of an engineered guide RNA of the present disclosure to a target RNA. An internal loop can be a symmetrical internal loop or an asymmetrical internal loop. A “symmetrical internal loop” is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetric internal loop of the present disclosure can be designated as a 5/5, 6/6, 7/7, 8/8, 9/9, 10/10, 11/11, 12/12, 13/13, 14/14, 15/15, 16/16, 17/17, 18/18, 19/19, 20/20, etc. symmetric internal loop, where the first number is the number of nucleotides contributed to the symmetric internal loop from the engineered guide RNA side of the guide-target RNA scaffold and the second number is the number of nucleotides contributed to the symmetric internal loop from the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 15 nucleotides on the engineered polynucleotide side of the guide-target RNA scaffold target and 15 nucleotides on the target RNA side of the guide-target RNA scaffold. A symmetrical internal loop of the present disclosure can be formed by 20 nucleotides on the engineered polynucleotide side of the guide-target RNA scaffold target and 20 nucleotides on the target RNA side of the guide-target RNA scaffold. A symmetrical internal loop of the present disclosure can be formed by 30 nucleotides on the engineered polynucleotide side of the guide-target RNA scaffold target and 30 nucleotides on the target RNA side of the guide-target RNA scaffold. A symmetrical internal loop of the present disclosure can be formed by 40 nucleotides on the engineered polynucleotide side of the guide-target RNA scaffold target and 40 nucleotides on the target RNA side of the guide-target RNA scaffold. A symmetrical internal loop of the present disclosure can be formed by 50 nucleotides on the engineered polynucleotide side of the guide-target RNA scaffold target and 50 nucleotides on the target RNA side of the guide-target RNA scaffold. A symmetrical internal loop of the present disclosure can be formed by 60 nucleotides on the engineered polynucleotide side of the guide-target RNA scaffold target and 60 nucleotides on the target RNA side of the guide-target RNA scaffold. A symmetrical internal loop of the present disclosure can be formed by 70 nucleotides on the engineered polynucleotide side of the guide-target RNA scaffold target and 70 nucleotides on the target RNA side of the guide-target RNA scaffold. A symmetrical internal loop of the present disclosure can be formed by 80 nucleotides on the engineered polynucleotide side of the guide-target RNA scaffold target and 80 nucleotides on the target RNA side of the guide-target RNA scaffold. A symmetrical internal loop of the present disclosure can be formed by 90 nucleotides on the engineered polynucleotide side of the guide-target RNA scaffold target and 90 nucleotides on the target RNA side of the guide-target RNA scaffold. A symmetrical internal loop of the present disclosure can be formed by 100 nucleotides on the engineered polynucleotide side of the guide-target RNA scaffold target and 100 nucleotides on the target RNA side of the guide-target RNA scaffold. A symmetrical internal loop of the present disclosure can be formed by 110 nucleotides on the engineered polynucleotide side of the guide-target RNA scaffold target and 110 nucleotides on the target RNA side of the guide-target RNA scaffold. A symmetrical internal loop of the present disclosure can be formed by 120 nucleotides on the engineered polynucleotide side of the guide-target RNA scaffold target and 120 nucleotides on the target RNA side of the guide-target RNA scaffold. A symmetrical internal loop of the present disclosure can be formed by 130 nucleotides on the engineered polynucleotide side of the guide-target RNA scaffold target and 130 nucleotides on the target RNA side of the guide-target RNA scaffold. A symmetrical internal loop of the present disclosure can be formed by 140 nucleotides on the engineered polynucleotide side of the guide-target RNA scaffold target and 140 nucleotides on the target RNA side of the guide-target RNA scaffold. A symmetrical internal loop of the present disclosure can be formed by 150 nucleotides on the engineered polynucleotide side of the guide-target RNA scaffold target and 150 nucleotides on the target RNA side of the guide-target RNA scaffold. A symmetrical internal loop of the present disclosure can be formed by 200 nucleotides on the engineered polynucleotide side of the guide-target RNA scaffold target and 200 nucleotides on the target RNA side of the guide-target RNA scaffold. A symmetrical internal loop of the present disclosure can be formed by 250 nucleotides on the engineered polynucleotide side of the guide-target RNA scaffold target and 250 nucleotides on the target RNA side of the guide-target RNA scaffold. A symmetrical internal loop of the present disclosure can be formed by 300 nucleotides on the engineered polynucleotide side of the guide-target RNA scaffold target and 300 nucleotides on the target RNA side of the guide-target RNA scaffold. A symmetrical internal loop of the present disclosure can be formed by 350 nucleotides on the engineered polynucleotide side of the guide-target RNA scaffold target and 350 nucleotides on the target RNA side of the guide-target RNA scaffold. A symmetrical internal loop of the present disclosure can be formed by 400 nucleotides on the engineered polynucleotide side of the guide-target RNA scaffold target and 400 nucleotides on the target RNA side of the guide-target RNA scaffold. A symmetrical internal loop of the present disclosure can be formed by 450 nucleotides on the engineered polynucleotide side of the guide-target RNA scaffold target and 450 nucleotides on the target RNA side of the guide-target RNA scaffold. A symmetrical internal loop of the present disclosure can be formed by 500 nucleotides on the engineered polynucleotide side of the guide-target RNA scaffold target and 500 nucleotides on the target RNA side of the guide-target RNA scaffold. A symmetrical internal loop of the present disclosure can be formed by 600 nucleotides on the engineered polynucleotide side of the guide-target RNA scaffold target and 600 nucleotides on the target RNA side of the guide-target RNA scaffold. A symmetrical internal loop of the present disclosure can be formed by 700 nucleotides on the engineered polynucleotide side of the guide-target RNA scaffold target and 700 nucleotides on the target RNA side of the guide-target RNA scaffold. A symmetrical internal loop of the present disclosure can be formed by 800 nucleotides on the engineered polynucleotide side of the guide-target RNA scaffold target and 800 nucleotides on the target RNA side of the guide-target RNA scaffold. A symmetrical internal loop of the present disclosure can be formed by 900 nucleotides on the engineered polynucleotide side of the guide-target RNA scaffold target and 900 nucleotides on the target RNA side of the guide-target RNA scaffold. A symmetrical internal loop of the present disclosure can be formed by 1000 nucleotides on the engineered polynucleotide side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

As described herein, a double stranded RNA (dsRNA) substrate (e.g., a guide-target RNA scaffold) is formed upon hybridization of an engineered guide RNA of the present disclosure to a target RNA. An internal loop can be a symmetrical internal loop or an asymmetrical internal loop. An “asymmetrical internal loop” is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetric internal loop of the present disclosure can be designated as a 5/6, 5/7, 5/8, 5/9, 5/10, 5/11, 5/12, 5/13, 5/14, 5/15, 5/16, 5/17, 5/18, 5/19, 5/20, 6/5, 6/7, 6/8, 6/9, 6/10, 6/11, 6/12, 6/13, 6/14, 6/15, 6/16, 6/17, 6/18, 6/19, 6/20, 7/5, 7/6, 7/8, 7/9, 7/10, 7/11, 7/12, 7/13, 7/14, 7/15, 7/16, 7/17, 7/18, 7/19, 7/20, 8/5, 8/6, 8/7, 8/9, 8/10, 8/11, 8/12, 8/13, 8/14, 8/15, 8/16, 8/17, 8/18, 8/19, 8/20, 9/5, 9/6, 9/7, 9/8, 9/10, 9/11, 9/12, 9/13, 9/14, 9/15, 9/16, 9/17, 9/18, 9/19, 9/20, 10/5, 10/6, 10/7, 10/8, 10/9, 10/11, 10/12, 10/13, 10/14, 10/15, 10/16, 10/17, 10/18, 10/19, 10/20, 11/5, 11/6, 11/7, 11/8, 11/9, 11/10, 11/12, 11/13, 11/14, 11/15, 11/16, 11/17, 11/18, 11/19, 11/20, 12/5, 12/6, 12/7, 12/8, 12/9, 12/10, 12/11, 12/13, 12/14, 12/15, 12/16, 12/17, 12/18, 12/19, 12/20, 13/5, 13/6, 13/7, 13/8, 13/9, 13/10, 13/11, 13/12, 13/14, 13/15, 13/16, 13/17, 13/18, 13/19, 13/20, 14/5, 14/6, 14/7, 14/8, 14/9, 14/10, 14/11, 14/12, 14/13, 14/15, 14/16, 14/17, 14/18, 14/19, 14/20, 15/5, 15/6, 15/7, 15/8, 15/9, 15/10, 15/11, 15/12, 15/13, 15/14, 15/16, 15/17, 15/18, 15/19, 15/20, 16/5, 16/6, 16/7, 16/8, 16/9, 16/10, 16/11, 16/12, 16/13, 16/14, 16/15, 16/17, 16/18, 16/19, 16/20, 17/5, 17/6, 17/7, 17/8, 17/9, 17/10, 17/11, 17/12, 17/13, 17/14, 17/15, 17/16, 17/18, 17/19, 17/20, 18/5, 18/6, 18/7, 18/8, 18/9, 18/10, 18/11, 18/12, 18/13, 18/14, 18/15, 18/16, 18/17, 18/19, 18/20, 19/5, 19/6, 19/7, 19/8, 19/9, 19/10, 19/11, 19/12, 19/13, 19/14, 19/15, 19/16, 19/17, 19/18, 19/20, 20/5, 20/6, 20/7, 20/8, 20/9, 20/10, 20/11, 20/12, 20/13, 20/14, 20/15, 20/16, 20/17, 20/18, 20/19, etc, asymmetric internal loop, the first number is the number of nucleotides contributed to the asymmetric internal loop from the engineered guide RNA side of the guide-target RNA scaffold and the second number is the number of nucleotides contributed to the asymmetric internal loop from the target RNA side of the guide-target RNA scaffold. An asymmetrical internal loop of the present disclosure can be formed by from 5 to 150 nucleotides on the engineered polynucleotide side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered polynucleotide side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered polynucleotide side of the guide-target RNA scaffold. An asymmetrical internal loop of the present disclosure can be formed by 5 nucleotides on the target RNA side of the guide-target RNA scaffold and 100 nucleotides on the engineered polynucleotide side of the guide-target RNA scaffold. An asymmetrical internal loop of the present disclosure can be formed by 5 nucleotides on the target RNA side of the guide-target RNA scaffold and 150 nucleotides on the engineered polynucleotide side of the guide-target RNA scaffold. An asymmetrical internal loop of the present disclosure can be formed by 5 nucleotides on the target RNA side of the guide-target RNA scaffold and 200 nucleotides on the engineered polynucleotide side of the guide-target RNA scaffold. An asymmetrical internal loop of the present disclosure can be formed by 5 nucleotides on the target RNA side of the guide-target RNA scaffold and 300 nucleotides on the engineered polynucleotide side of the guide-target RNA scaffold. An asymmetrical internal loop of the present disclosure can be formed by 5 nucleotides on the target RNA side of the guide-target RNA scaffold and 400 nucleotides on the engineered polynucleotide side of the guide-target RNA scaffold. An asymmetrical internal loop of the present disclosure can be formed by 5 nucleotides on the target RNA side of the guide-target RNA scaffold and 500 nucleotides on the engineered polynucleotide side of the guide-target RNA scaffold. An asymmetrical internal loop of the present disclosure can be formed by 5 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 5 nucleotides on the engineered polynucleotide side of the guide-target RNA scaffold. An asymmetrical internal loop of the present disclosure can be formed by 500 nucleotides on the target RNA side of the guide-target RNA scaffold and 5 nucleotides on the engineered polynucleotide side of the guide-target RNA scaffold. An asymmetrical internal loop of the present disclosure can be formed by 400 nucleotides on the target RNA side of the guide-target RNA scaffold and 5 nucleotides on the engineered polynucleotide side of the guide-target RNA scaffold. An asymmetrical internal loop of the present disclosure can be formed by 300 nucleotides on the target RNA side of the guide-target RNA scaffold and 5 nucleotides on the engineered polynucleotide side of the guide-target RNA scaffold. An asymmetrical internal loop of the present disclosure can be formed by 200 nucleotides on the target RNA side of the guide-target RNA scaffold and 5 nucleotides on the engineered polynucleotide side of the guide-target RNA scaffold. An asymmetrical internal loop of the present disclosure can be formed by 150 nucleotides on the target RNA side of the guide-target RNA scaffold and 5 nucleotides on the engineered polynucleotide side of the guide-target RNA scaffold. An asymmetrical internal loop of the present disclosure can be formed by 100 nucleotides on the target RNA side of the guide-target RNA scaffold and 5 nucleotides on the engineered polynucleotide side of the guide-target RNA scaffold. An asymmetrical internal loop of the present disclosure can be formed by 50 nucleotides on the target RNA side of the guide-target RNA scaffold and 5 nucleotides on the engineered polynucleotide side of the guide-target RNA scaffold. An asymmetrical internal loop of the present disclosure can be formed by 50 nucleotides on the target RNA side of the guide-target RNA scaffold and 100 nucleotides on the engineered polynucleotide side of the guide-target RNA scaffold. An asymmetrical internal loop of the present disclosure can be formed by 50 nucleotides on the target RNA side of the guide-target RNA scaffold and 150 nucleotides on the engineered polynucleotide side of the guide-target RNA scaffold. An asymmetrical internal loop of the present disclosure can be formed by 50 nucleotides on the target RNA side of the guide-target RNA scaffold and 200 nucleotides on the engineered polynucleotide side of the guide-target RNA scaffold. An asymmetrical internal loop of the present disclosure can be formed by 50 nucleotides on the target RNA side of the guide-target RNA scaffold and 300 nucleotides on the engineered polynucleotide side of the guide-target RNA scaffold. An asymmetrical internal loop of the present disclosure can be formed by 50 nucleotides on the target RNA side of the guide-target RNA scaffold and 400 nucleotides on the engineered polynucleotide side of the guide-target RNA scaffold. An asymmetrical internal loop of the present disclosure can be formed by 50 nucleotides on the target RNA side of the guide-target RNA scaffold and 500 nucleotides on the engineered polynucleotide side of the guide-target RNA scaffold. An asymmetrical internal loop of the present disclosure can be formed by 50 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 50 nucleotides on the engineered polynucleotide side of the guide-target RNA scaffold. An asymmetrical internal loop of the present disclosure can be formed by 500 nucleotides on the target RNA side of the guide-target RNA scaffold and 50 nucleotides on the engineered polynucleotide side of the guide-target RNA scaffold. An asymmetrical internal loop of the present disclosure can be formed by 400 nucleotides on the target RNA side of the guide-target RNA scaffold and 50 nucleotides on the engineered polynucleotide side of the guide-target RNA scaffold. An asymmetrical internal loop of the present disclosure can be formed by 300 nucleotides on the target RNA side of the guide-target RNA scaffold and 50 nucleotides on the engineered polynucleotide side of the guide-target RNA scaffold. An asymmetrical internal loop of the present disclosure can be formed by 200 nucleotides on the target RNA side of the guide-target RNA scaffold and 50 nucleotides on the engineered polynucleotide side of the guide-target RNA scaffold. An asymmetrical internal loop of the present disclosure can be formed by 150 nucleotides on the target RNA side of the guide-target RNA scaffold and 50 nucleotides on the engineered polynucleotide side of the guide-target RNA scaffold. An asymmetrical internal loop of the present disclosure can be formed by 100 nucleotides on the target RNA side of the guide-target RNA scaffold and 50 nucleotides on the engineered polynucleotide side of the guide-target RNA scaffold. An asymmetrical internal loop of the present disclosure can be formed by 100 nucleotides on the target RNA side of the guide-target RNA scaffold and 150 nucleotides on the engineered polynucleotide side of the guide-target RNA scaffold. An asymmetrical internal loop of the present disclosure can be formed by 100 nucleotides on the target RNA side of the guide-target RNA scaffold and 200 nucleotides on the engineered polynucleotide side of the guide-target RNA scaffold. An asymmetrical internal loop of the present disclosure can be formed by 100 nucleotides on the target RNA side of the guide-target RNA scaffold and 300 nucleotides on the engineered polynucleotide side of the guide-target RNA scaffold. An asymmetrical internal loop of the present disclosure can be formed by 100 nucleotides on the target RNA side of the guide-target RNA scaffold and 400 nucleotides on the engineered polynucleotide side of the guide-target RNA scaffold. An asymmetrical internal loop of the present disclosure can be formed by 100 nucleotides on the target RNA side of the guide-target RNA scaffold and 500 nucleotides on the engineered polynucleotide side of the guide-target RNA scaffold. An asymmetrical internal loop of the present disclosure can be formed by 100 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 100 nucleotides on the engineered polynucleotide side of the guide-target RNA scaffold. An asymmetrical internal loop of the present disclosure can be formed by 500 nucleotides on the target RNA side of the guide-target RNA scaffold and 100 nucleotides on the engineered polynucleotide side of the guide-target RNA scaffold. An asymmetrical internal loop of the present disclosure can be formed by 400 nucleotides on the target RNA side of the guide-target RNA scaffold and 100 nucleotides on the engineered polynucleotide side of the guide-target RNA scaffold. An asymmetrical internal loop of the present disclosure can be formed by 300 nucleotides on the target RNA side of the guide-target RNA scaffold and 100 nucleotides on the engineered polynucleotide side of the guide-target RNA scaffold. An asymmetrical internal loop of the present disclosure can be formed by 200 nucleotides on the target RNA side of the guide-target RNA scaffold and 100 nucleotides on the engineered polynucleotide side of the guide-target RNA scaffold. An asymmetrical internal loop of the present disclosure can be formed by 150 nucleotides on the target RNA side of the guide-target RNA scaffold and 100 nucleotides on the engineered polynucleotide side of the guide-target RNA scaffold. An asymmetrical internal loop of the present disclosure can be formed by 150 nucleotides on the target RNA side of the guide-target RNA scaffold and 200 nucleotides on the engineered polynucleotide side of the guide-target RNA scaffold. An asymmetrical internal loop of the present disclosure can be formed by 150 nucleotides on the target RNA side of the guide-target RNA scaffold and 300 nucleotides on the engineered polynucleotide side of the guide-target RNA scaffold. An asymmetrical internal loop of the present disclosure can be formed by 150 nucleotides on the target RNA side of the guide-target RNA scaffold and 400 nucleotides on the engineered polynucleotide side of the guide-target RNA scaffold. An asymmetrical internal loop of the present disclosure can be formed by 150 nucleotides on the target RNA side of the guide-target RNA scaffold and 500 nucleotides on the engineered polynucleotide side of the guide-target RNA scaffold. An asymmetrical internal loop of the present disclosure can be formed by 150 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 150 nucleotides on the engineered polynucleotide side of the guide-target RNA scaffold. An asymmetrical internal loop of the present disclosure can be formed by 500 nucleotides on the target RNA side of the guide-target RNA scaffold and 5 nucleotides on the engineered polynucleotide side of the guide-target RNA scaffold. An asymmetrical internal loop of the present disclosure can be formed by 400 nucleotides on the target RNA side of the guide-target RNA scaffold and 150 nucleotides on the engineered polynucleotide side of the guide-target RNA scaffold. An asymmetrical internal loop of the present disclosure can be formed by 300 nucleotides on the target RNA side of the guide-target RNA scaffold and 150 nucleotides on the engineered polynucleotide side of the guide-target RNA scaffold. An asymmetrical internal loop of the present disclosure can be formed by 200 nucleotides on the target RNA side of the guide-target RNA scaffold and 300 nucleotides on the engineered polynucleotide side of the guide-target RNA scaffold. An asymmetrical internal loop of the present disclosure can be formed by 200 nucleotides on the target RNA side of the guide-target RNA scaffold and 400 nucleotides on the engineered polynucleotide side of the guide-target RNA scaffold. An asymmetrical internal loop of the present disclosure can be formed by 200 nucleotides on the target RNA side of the guide-target RNA scaffold and 500 nucleotides on the engineered polynucleotide side of the guide-target RNA scaffold. An asymmetrical internal loop of the present disclosure can be formed by 200 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 200 nucleotides on the engineered polynucleotide side of the guide-target RNA scaffold. An asymmetrical internal loop of the present disclosure can be formed by 500 nucleotides on the target RNA side of the guide-target RNA scaffold and 200 nucleotides on the engineered polynucleotide side of the guide-target RNA scaffold. An asymmetrical internal loop of the present disclosure can be formed by 400 nucleotides on the target RNA side of the guide-target RNA scaffold and 200 nucleotides on the engineered polynucleotide side of the guide-target RNA scaffold. An asymmetrical internal loop of the present disclosure can be formed by 300 nucleotides on the target RNA side of the guide-target RNA scaffold and 200 nucleotides on the engineered polynucleotide side of the guide-target RNA scaffold. An asymmetrical internal loop of the present disclosure can be formed by 300 nucleotides on the target RNA side of the guide-target RNA scaffold and 400 nucleotides on the engineered polynucleotide side of the guide-target RNA scaffold. An asymmetrical internal loop of the present disclosure can be formed by 300 nucleotides on the target RNA side of the guide-target RNA scaffold and 500 nucleotides on the engineered polynucleotide side of the guide-target RNA scaffold. An asymmetrical internal loop of the present disclosure can be formed by 300 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 300 nucleotides on the engineered polynucleotide side of the guide-target RNA scaffold. An asymmetrical internal loop of the present disclosure can be formed by 500 nucleotides on the target RNA side of the guide-target RNA scaffold and 300 nucleotides on the engineered polynucleotide side of the guide-target RNA scaffold. An asymmetrical internal loop of the present disclosure can be formed by 400 nucleotides on the target RNA side of the guide-target RNA scaffold and 300 nucleotides on the engineered polynucleotide side of the guide-target RNA scaffold. An asymmetrical internal loop of the present disclosure can be formed by 400 nucleotides on the target RNA side of the guide-target RNA scaffold and 500 nucleotides on the engineered polynucleotide side of the guide-target RNA scaffold. An asymmetrical internal loop of the present disclosure can be formed by 400 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 400 nucleotides on the engineered polynucleotide side of the guide-target RNA scaffold. An asymmetrical internal loop of the present disclosure can be formed by 500 nucleotides on the target RNA side of the guide-target RNA scaffold and 400 nucleotides on the engineered polynucleotide side of the guide-target RNA scaffold. An asymmetrical internal loop of the present disclosure can be formed by 500 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 500 nucleotides on the engineered polynucleotide side of the guide-target RNA scaffold. Thus, an asymmetrical internal loop can be a structural feature formed from latent structure provided by an engineered latent guide RNA.

In some embodiments, a first internal loop or a second internal loop can independently comprise a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the engineered guide RNA and a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the target RNA.

In some embodiments, an engineered guide RNA comprising a barbell macro-footprint (e.g., a latent structure that manifests as a first internal loop and a second internal loop) comprises a cytosine in a micro-footprint sequence in between the macro-footprint sequence that, when the engineered guide RNA is hybridized to the target RNA, is present in the guide-target RNA scaffold opposite an adenosine that is edited by the RNA editing entity (e.g., an on-target adenosine). In such embodiments, the cytosine of the micro-footprint is comprised in an A/C mismatch with the on-target adenosine of the target RNA in the guide-target RNA scaffold.

A first internal loop and a second internal loop of the barbell macro-footprint can be positioned a certain distance from the A/C mismatch, with respect to the base of the first internal loop and the base of the second internal loop that is the most proximal to the A/C mismatch. In some embodiments, the first internal loop and the second internal loop can be positioned the same number of bases from the A/C mismatch, with respect to the base of the first internal loop and the base of the second internal loop that is the most proximal to the A/C mismatch. In some embodiments, the first internal loop and the second internal loop can be positioned a different number of bases from the A/C mismatch, with respect to the base of the first internal loop and the base of the second internal loop that is the most proximal to the A/C mismatch.

In some embodiments, the first internal loop of the barbell or the second internal loop of the barbell can be positioned at least about 5 bases (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases) away from the A/C mismatch with respect to the base of the first internal loop or the second internal loop that is the most proximal to the A/C mismatch. In some embodiments, the first internal loop of the barbell or the second internal loop of the barbell can be positioned at most about 50 bases away from the A/C mismatch (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5) with respect to the base of the first internal loop or the second internal loop that is the most proximal to the A/C mismatch.

In some embodiments, the first internal loop can be positioned from about 1 base away from the A/C mismatch to about 30 bases away from the A/C mismatch (e.g., 1-20, 7-30, 5-20, 6-20, 5-15) with respect to the base of the first internal loop that is most proximal to the A/C mismatch. In some embodiments, the first internal loop can be positioned from about 5 bases away from the A/C mismatch to about 15 bases away from the A/C mismatch (e.g., 5-15, 6-14, 7-13, 8-12, 9-11) with respect to the base of the first internal loop that is most proximal to the A/C mismatch. In some examples, the first internal loop can be positioned from about 9 bases away from the A/C mismatch to about 15 bases away from the A/C mismatch (e.g., 10-14, 11-13) with respect to the base of the first internal loop that is the most proximal to the A/C mismatch.

In some embodiments, the second internal loop can be positioned from about 12 bases away from the A/C mismatch to about 40 bases away from the A/C mismatch (e.g., 13-39, 14-38, 15-40, 15-38, 15-37, 16-36, 17-35, 18-38, 18-34, 19-33, 20-32, 21-31, 22-30, 23-29, 24-28, 25-27) with respect to the base of the second internal loop that is the most proximal to the A/C mismatch. In some embodiments, the second internal loop can be positioned from about 20 bases away from the A/C mismatch to about 33 bases away from the A/C mismatch with respect to the base of the second internal loop that is most proximal to the A/C mismatch. In some embodiments, the second internal loop can be positioned 24, 26, 28, or 30 bases away from the A/C mismatch with respect to the base of the second internal loop that is most proximal to the A/C mismatch. In some embodiments, the first internal loop can be positioned at least about 1 base away from the on-target adenosine of the target RNA. In some embodiments, the first internal loop can be positioned about 1 base away from the on-target adenosine of the target RNA to about 30 bases away from the on-target adenosine of the target RNA. In some embodiments, the first internal loop can be positioned about 1 base away from the on-target adenosine of the target RNA to about 20 bases away from the on-target adenosine of the target RNA. In some embodiments, the first internal loop can be positioned at least about 5 bases away from the on-target adenosine of the target RNA. In some embodiments, the first internal loop can be positioned about 5 bases away from the on-target adenosine of the target RNA to about 15 bases away from the on-target adenosine of the target RNA. In some embodiments, the first internal loop can be positioned at least about 6 bases away from the on-target adenosine of the target RNA. In some embodiments, the first internal loop can be positioned about 6 bases away from the on-target adenosine of the target RNA to about 20 bases away from the on-target adenosine of the target RNA. In some embodiments, the first internal loop can be positioned at least about 7 bases away from the on-target adenosine of the target RNA. In some embodiments, the first internal loop can be positioned about 7 bases away from the on-target adenosine of the target RNA to about 30 bases away from the on-target adenosine of the target RNA. In some embodiments, the first internal loop can be positioned about 9 bases away from the on-target adenosine of the target RNA to about 15 bases away from the on-target adenosine of the target RNA. In some embodiments, the first internal loop can be positioned about 6 bases away from the on-target adenosine of the target RNA. In some embodiments, the first internal loop can be positioned about 10 bases away from the on-target adenosine of the target RNA. In some embodiments, the first internal loop can be positioned about 12 bases away from the on-target adenosine of the target RNA. In some embodiments, the first internal loop can be positioned about 15 bases away from the on-target adenosine of the target RNA. In some embodiments, the second internal loop can be positioned at least about 12 bases away from the on-target adenosine of the target RNA. In some embodiments, the second internal loop can be positioned about 12 bases away from the on-target adenosine of the target RNA to about 40 bases away from the on-target adenosine of the target RNA. In some embodiments, the second internal loop can be positioned at least about 15 bases away from the on-target adenosine of the target RNA. In some embodiments, the second internal loop can be positioned about 15 bases away from the on-target adenosine of the target RNA to about 40 bases away from the on-target adenosine of the target RNA. In some embodiments, the second internal loop can be positioned about 15 bases away from the on-target adenosine of the target RNA to about 38 bases away from the on-target adenosine of the target RNA. In some embodiments, the second internal loop can be positioned at least about 18 bases away from the on-target adenosine of the target RNA. In some embodiments, the second internal loop can be positioned about 18 bases away from the on-target adenosine of the target RNA to about 38 bases away from the on-target adenosine of the target RNA. In some embodiments, the second internal loop can be positioned about 18 bases away from the on-target adenosine of the target RNA to about 35 bases away from the on-target adenosine of the target RNA. In some embodiments, the second internal loop can be positioned about 20 bases away from the on-target adenosine of the target RNA to about 33 bases away from the on-target adenosine of the target RNA. In some embodiments, the second internal loop can be positioned about 24 bases away from the on-target adenosine of the target RNA. In some embodiments, the second internal loop can be positioned about 33 bases away from the on-target adenosine of the target RNA. In some embodiments, the second internal loop can be positioned about 34 bases away from the on-target adenosine of the target RNA. In some embodiments, the first internal loop can be positioned about 7 bases away from the on-target adenosine of the target RNA to about 30 bases away from the on-target adenosine of the target RNA and the second internal loop can be positioned about 18 bases away from the on-target adenosine of the target RNA to about 34 bases away from the on-target adenosine of the target RNA. In some embodiments, the first internal loop can be positioned about 5 bases away from the on-target adenosine of the target RNA to about 15 bases away from the on-target adenosine of the target RNA and the second internal loop can be positioned about 12 bases away from the on-target adenosine of the target RNA to about 40 bases away from the on-target adenosine of the target RNA. In some embodiments, the first internal loop can be positioned about 6 bases away from the on-target adenosine of the target RNA to about 20 bases away from the on-target adenosine of the target RNA and the second internal loop can be positioned about 15 bases away from the on-target adenosine of the target RNA to about 38 bases away from the on-target adenosine of the target RNA. In some embodiments, the first internal loop can be positioned about 5 bases away from the on-target adenosine of the target RNA to about 20 bases away from the on-target adenosine of the target RNA and the second internal loop can be positioned about 18 bases away from the on-target adenosine of the target RNA to about 38 bases away from the on-target adenosine of the target RNA. In some embodiments, the first internal loop can be positioned about 5 bases away from the on-target adenosine of the target RNA to about 15 bases away from the on-target adenosine of the target RNA and the second internal loop can be positioned about 18 bases away from the on-target adenosine of the target RNA to about 38 bases away from the on-target adenosine of the target RNA. In some embodiments, the first internal loop can be positioned about 1 base away from the on-target adenosine of the target RNA to about 20 bases away from the on-target adenosine of the target RNA and the second internal loop can be positioned about 12 bases away from the on-target adenosine of the target RNA to about 40 bases away from the on-target adenosine of the target RNA. In some embodiments, the first internal loop can be positioned about 5 bases away from the on-target adenosine of the target RNA to about 20 bases away from the on-target adenosine of the target RNA and the second internal loop can be positioned about 12 bases away from the on-target adenosine of the target RNA to about 40 bases away from the on-target adenosine of the target RNA. In some embodiments, the first internal loop can be positioned about 5 bases away from the on-target adenosine of the target RNA to about 20 bases away from the on-target adenosine of the target RNA and the second internal loop can be positioned about 15 bases away from the on-target adenosine of the target RNA to about 40 bases away from the on-target adenosine of the target RNA. In some embodiments, the first internal loop can be positioned about 10 bases away from the on-target adenosine of the target RNA and the second internal loop can be positioned about 34 bases away from the on-target adenosine of the target RNA. In some embodiments, the first internal loop can be positioned about 15 bases away from the on-target adenosine of the target RNA and the second internal loop can be positioned about 33 bases away from the on-target adenosine of the target RNA. In some embodiments, the first internal loop can be positioned about 6 bases away from the on-target adenosine of the target RNA and the second internal loop can be positioned about 34 bases away from the on-target adenosine of the target RNA. In some embodiments, the first internal loop can be positioned about 12 bases away from the on-target adenosine of the target RNA and the second internal loop can be positioned about 34 bases away from the on-target adenosine of the target RNA. In some embodiments, the first internal loop can be positioned about 6 bases away from the on-target adenosine of the target RNA and the second internal loop can be positioned about 33 bases away from the on-target adenosine of the target RNA. In some embodiments, the first internal loop can be positioned about 12 bases away from the on-target adenosine of the target RNA and the second internal loop can be positioned about 24 bases away from the on-target adenosine of the target RNA. In some embodiments, the first internal loop can be positioned about 10 bases away from the on-target adenosine of the target RNA and the second internal loop can be positioned about 33 bases away from the on-target adenosine of the target RNA. In some embodiments, the engineered guide RNA can comprise a cytosine that, when the engineered guide RNA is hybridized to the target RNA, is present in the guide-target RNA scaffold opposite the on-target adenosine that is edited by the RNA editing entity, thereby forming an A/C mismatch in the double stranded RNA substrate. In some embodiments, the first internal loop and the second internal loop can be positioned the same number of bases from the A/C mismatch with respect to the base of the first internal loop and the base of the second internal loop that is most proximal to the A/C mismatch. In some embodiments, the first internal loop can be positioned at least about 1 base away from the A/C mismatch with respect to the base of the first internal loop that is most proximal to the A/C mismatch. In some embodiments, the first internal loop can be positioned about 1 base away from the A/C mismatch to about 20 bases away from the A/C mismatch with respect to the base of the first internal loop that is most proximal to the A/C mismatch. In some embodiments, the first internal loop can be positioned at least about 5 bases away from the A/C mismatch with respect to the base of the first internal loop that is most proximal to the A/C mismatch. In some embodiments, the first internal loop can be positioned about 5 bases away from the A/C mismatch to about 20 bases away from the A/C mismatch with respect to the base of the first internal loop that is most proximal to the A/C mismatch. In some embodiments, the first internal loop can be positioned about 5 bases away from the A/C mismatch to about 15 bases away from the A/C mismatch with respect to the base of the first internal loop that is most proximal to the A/C mismatch. In some embodiments, the first internal loop can be positioned at least about 6 bases away from the A/C mismatch with respect to the base of the first internal loop that is most proximal to the A/C mismatch. In some embodiments, the first internal loop can be positioned about 6 bases away from the A/C mismatch to about 20 bases away from the A/C mismatch with respect to the base of the first internal loop that is most proximal to the A/C mismatch. In some embodiments, the first internal loop can be positioned at least about 7 bases away from the A/C mismatch with respect to the base of the first internal loop that is most proximal to the A/C mismatch. In some embodiments, the first internal loop can be positioned about 7 bases away from the A/C mismatch to about 30 bases away from the A/C mismatch with respect to the base of the first internal loop that is most proximal to the A/C mismatch. In some embodiments, the first internal loop can be positioned about 9 bases away from the A/C mismatch to about 15 bases away from the A/C mismatch with respect to the base of the first internal loop that is most proximal to the A/C mismatch. In some embodiments, the second internal loop can be positioned at least about 12 bases away from the A/C mismatch with respect to the base of the second internal loop that is most proximal to the A/C mismatch. In some embodiments, the second internal loop can be positioned about 12 bases away from the A/C mismatch to about 40 bases away from the A/C mismatch with respect to the base of the second internal loop that is most proximal to the A/C mismatch. In some embodiments, the second internal loop can be positioned at least about 15 bases away from the A/C mismatch with respect to the base of the second internal loop that is most proximal to the A/C mismatch. In some embodiments, the second internal loop can be positioned about 15 bases away from the A/C mismatch to about 40 bases away from the A/C mismatch with respect to the base of the second internal loop that is most proximal to the A/C mismatch. In some embodiments, the second internal loop can be positioned about 15 bases away from the A/C mismatch to about 38 bases away from the A/C mismatch with respect to the base of the second internal loop that is most proximal to the A/C mismatch. In some embodiments, the second internal loop can be positioned at least about 18 bases away from the A/C mismatch with respect to the base of the second internal loop that is most proximal to the A/C mismatch. In some embodiments, the second internal loop can be positioned about 18 bases away from the A/C mismatch to about 38 bases away from the A/C mismatch with respect to the base of the second internal loop that is most proximal to the A/C mismatch. In some embodiments, the second internal loop can be positioned about 18 bases away from the A/C mismatch to about 35 bases away from the A/C mismatch with respect to the base of the second internal loop that is most proximal to the A/C mismatch. In some embodiments, the second internal loop can be positioned about 20 bases away from the A/C mismatch to about 33 bases away from the A/C mismatch with respect to the base of the second internal loop that is most proximal to the A/C mismatch. In some embodiments, the second internal loop can be positioned about 24 bases away from the A/C mismatch with respect to the base of the second internal loop that is most proximal to the A/C mismatch. In some embodiments, the second internal loop can be positioned about 33 bases away from the A/C mismatch with respect to the base of the second internal loop that is most proximal to the A/C mismatch. In some embodiments, the second internal loop can be positioned about 34 bases away from the A/C mismatch with respect to the base of the second internal loop that is most proximal to the A/C mismatch. In some embodiments, the first internal loop can be positioned about 10 bases away from the A/C mismatch with respect to the base of the first internal loop that is most proximal to the A/C mismatch and the second internal loop can be positioned about 34 bases away from the A/C mismatch with respect to the base of the second internal loop that is most proximal to the A/C mismatch. In some embodiments, the first internal loop can be positioned about 15 bases away from the A/C mismatch with respect to the base of the first internal loop that is most proximal to the A/C mismatch and the second internal loop can be positioned about 33 bases away from the A/C mismatch with respect to the base of the second internal loop that is most proximal to the A/C mismatch. In some embodiments, the first internal loop can be positioned about 6 bases away from the A/C mismatch with respect to the base of the first internal loop that is most proximal to the A/C mismatch and the second internal loop can be positioned about 34 bases away from the A/C mismatch with respect to the base of the second internal loop that is most proximal to the A/C mismatch. In some embodiments, the first internal loop can be positioned about 12 bases away from the A/C mismatch with respect to the base of the first internal loop that is most proximal to the A/C mismatch and the second internal loop can be positioned about 34 bases away from the A/C mismatch with respect to the base of the second internal loop that is most proximal to the A/C mismatch. In some embodiments, the first internal loop can be positioned about 6 bases away from the A/C mismatch with respect to the base of the first internal loop that is most proximal to the A/C mismatch and the second internal loop can be positioned about 33 bases away from the A/C mismatch with respect to the base of the second internal loop that is most proximal to the A/C mismatch. In some embodiments, the first internal loop can be positioned about 12 bases away from the A/C mismatch with respect to the base of the first internal loop that is most proximal to the A/C mismatch and the second internal loop can be positioned about 24 bases away from the A/C mismatch with respect to the base of the second internal loop that is most proximal to the A/C mismatch. In some embodiments, the first internal loop can be positioned about 10 bases away from the A/C mismatch with respect to the base of the first internal loop that is most proximal to the A/C mismatch and the second internal loop can be positioned about 33 bases away from the A/C mismatch with respect to the base of the second internal loop that is most proximal to the A/C mismatch.

In some embodiments, a target RNA can be an ABCA4 RNA. In this example, an engineered guide RNA comprising a barbell macro-footprint sequence, upon hybridization with the ABCA4 mRNA, forms a guide-target RNA scaffold with the ABCA4 RNA. The ABCA4 guide-target RNA scaffold when present comprises a right internal loop (e.g., a right barbell) and a left internal loop (e.g., a left barbell) manifested from the barbell macro-footprint sequence.

In some embodiments, a guide RNA targeting ABCA4 can comprise a first internal loop and a second internal loop independently positioned as follows: the first internal loop is positioned at a distance of about 2 bases or greater upstream of the on-target adenosine (e.g., 3, 4, 5, 6, 7, 8, 9, 10, 11, 12, 13, 14, 15, 16, 17, 18, 19, 20), about 20 bases or fewer upstream of the on-target adenosine (e.g., 19, 18, 17, 16, 15, 14, 13, 12, 11, 10, 9, 8, 7, 6, 5, 4, 3, 2), or from about 2 bases to about 20 bases upstream of the on-target adenosine (e.g., 3-19, 4-18, 5-17, 6-16, 7-15, 8-14, 9-13, 10-12); and the second internal loop is positioned at a distance of about 12 bases or greater downstream of the on-target adenosine (e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40), about 40 bases or fewer downstream of the on-target adenosine (e.g., 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2), or from about 12 bases to about 40 bases downstream of the on-target adenosine (e.g., 13-39, 14-38, 15-37, 16-36, 17-35, 18-34, 19-33, 20-32, 21-31, 22-30, 23-29, 24-28, 25-27).

In some embodiments, a guide RNA targeting ABCA4 can comprise: a first internal loop positioned about 5 bases upstream of the on-target adenosine and a second internal loop positioned about 27 bases downstream of the on-target adenosine; a first internal loop positioned about 5 bases upstream of the on-target adenosine and a second internal loop positioned about 32 bases downstream of the on-target adenosine; a first internal loop positioned about 5 bases upstream of the on-target adenosine and a second internal loop positioned about 33 bases downstream of the on-target adenosine; a first internal loop positioned about 9 bases upstream of the on-target adenosine and a second internal loop positioned about 33 bases downstream of the on-target adenosine; a first internal loop positioned about 10 bases upstream of the on-target adenosine and a second internal loop positioned about 33 bases downstream of the on-target adenosine; a first internal loop positioned about 13 bases upstream of the on-target adenosine and a second internal loop positioned about 33 bases downstream of the on-target adenosine; a first internal loop positioned about 14 bases upstream of the on-target adenosine and a second internal loop positioned about 33 bases downstream of the on-target adenosine; or a first internal loop positioned about 15 bases upstream of the on-target adenosine and a second internal loop positioned about 33 bases downstream of the on-target adenosine.

In some embodiments, an engineered guide RNA targeting ABCA4 can comprise a first internal loop positioned at a distance of about 15 bases upstream of the target adenosine to be edited and a second internal loop positioned at a distance of about 33 bases downstream of the target adenosine to be edited. Said engineered guide RNAs can exhibit superior on-target editing and low off-target editing, resulting in less than about 3% off-target editing. Thus, an engineered guide RNA targeting ABCA4 and forming a barbell macro-footprint, where the first internal loop is at the −15 position and the second internal loop is at the +33 position can be highly efficient and specific, with about 40% or more on-target editing and less than about 3% off target editing by ADAR.

In some embodiments, a target RNA can be an GAPDH RNA. In this example, an engineered guide RNA comprising a barbell macro-footprint sequence, upon hybridization with the GAPDH mRNA, forms a guide-target RNA scaffold with the GAPDH RNA. The GAPDH guide-target RNA scaffold when present comprises a right internal loop (e.g., a right barbell) and a left internal loop (e.g., a left barbell) manifested from the barbell macro-footprint sequence.

In some embodiments, a guide RNA targeting GAPDH can comprise a first internal loop and a second internal loop independently positioned as follows: the first internal loop is positioned at a distance of about 2 bases or greater upstream of the on-target adenosine (e.g., 3, 4, 5, 6, 7, 8, 9, 10, 11, 12, 13, 14, 15, 16, 17, 18, 19, 20), about 20 bases or fewer upstream of the on-target adenosine (e.g., 19, 18, 17, 16, 15, 14, 13, 12, 11, 10, 9, 8, 7, 6, 5, 4, 3, 2), or from about 2 bases to about 20 bases upstream of the on-target adenosine (e.g., 3-19, 4-18, 5-17, 6-16, 7-15, 8-14, 9-13, 10-12); and the second internal loop is positioned at a distance of about 12 bases or greater downstream of the on-target adenosine (e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40), about 40 bases or fewer downstream of the on-target adenosine (e.g., 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2), or from about 12 bases to about 40 bases downstream of the on-target adenosine (e.g., 13-39, 14-38, 15-37, 16-36, 17-35, 18-34, 19-33, 20-32, 21-31, 22-30, 23-29, 24-28, 25-27).

In some embodiments, a guide RNA targeting GAPDH can comprise: a first internal loop positioned about 5 bases upstream of the on-target adenosine and a second internal loop positioned about 27 bases downstream of the on-target adenosine; a first internal loop positioned about 5 bases upstream of the on-target adenosine and a second internal loop positioned about 32 bases downstream of the on-target adenosine; a first internal loop positioned about 5 bases upstream of the on-target adenosine and a second internal loop positioned about 33 bases downstream of the on-target adenosine; a first internal loop positioned about 9 bases upstream of the on-target adenosine and a second internal loop positioned about 33 bases downstream of the on-target adenosine; a first internal loop positioned about 10 bases upstream of the on-target adenosine and a second internal loop positioned about 33 bases downstream of the on-target adenosine; a first internal loop positioned about 13 bases upstream of the on-target adenosine and a second internal loop positioned about 33 bases downstream of the on-target adenosine; a first internal loop positioned about 14 bases upstream of the on-target adenosine and a second internal loop positioned about 33 bases downstream of the on-target adenosine; or a first internal loop positioned about 15 bases upstream of the on-target adenosine and a second internal loop positioned about 33 bases downstream of the on-target adenosine.

In some embodiments, a target RNA can be an MAPT RNA. In this example, an engineered guide RNA comprising a barbell macro-footprint sequence, upon hybridization with the MAPT mRNA, forms a guide-target RNA scaffold with the MAPT RNA. The MAPT guide-target RNA scaffold when present comprises a right internal loop (e.g., a right barbell) and a left internal loop (e.g., a left barbell) manifested from the barbell macro-footprint sequence.

In some embodiments, a guide RNA targeting MAPT can comprise a first internal loop and a second internal loop independently positioned as follows: the first internal loop is positioned at a distance of about 2 bases or greater upstream of the on-target adenosine (e.g., 3, 4, 5, 6, 7, 8, 9, 10, 11, 12, 13, 14, 15, 16, 17, 18, 19, 20), about 20 bases or fewer upstream of the on-target adenosine (e.g., 19, 18, 17, 16, 15, 14, 13, 12, 11, 10, 9, 8, 7, 6, 5, 4, 3, 2), or from about 2 bases to about 20 bases upstream of the on-target adenosine (e.g., 3-19, 4-18, 5-17, 6-16, 7-15, 8-14, 9-13, 10-12); and the second internal loop is positioned at a distance of about 12 bases or greater downstream of the on-target adenosine (e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40), about 40 bases or fewer downstream of the on-target adenosine (e.g., 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2), or from about 12 bases to about 40 bases downstream of the on-target adenosine (e.g., 13-39, 14-38, 15-37, 16-36, 17-35, 18-34, 19-33, 20-32, 21-31, 22-30, 23-29, 24-28, 25-27), or 24 bases, 26 bases, 28 bases, or 30 bases downstream of the on-target adenosine of the target RNA.

In some embodiments, a guide RNA targeting MAPT can comprise: a first internal loop positioned about 5 bases upstream of the on-target adenosine and a second internal loop positioned about 27 bases downstream of the on-target adenosine; a first internal loop positioned about 5 bases upstream of the on-target adenosine and a second internal loop positioned about 32 bases downstream of the on-target adenosine; a first internal loop positioned about 5 bases upstream of the on-target adenosine and a second internal loop positioned about 33 bases downstream of the on-target adenosine; a first internal loop positioned about 9 bases upstream of the on-target adenosine and a second internal loop positioned about 33 bases downstream of the on-target adenosine; a first internal loop positioned about 10 bases upstream of the on-target adenosine and a second internal loop positioned about 33 bases downstream of the on-target adenosine; a first internal loop positioned about 13 bases upstream of the on-target adenosine and a second internal loop positioned about 33 bases downstream of the on-target adenosine; a first internal loop positioned about 14 bases upstream of the on-target adenosine and a second internal loop positioned about 33 bases downstream of the on-target adenosine; or a first internal loop positioned about 15 bases upstream of the on-target adenosine and a second internal loop positioned about 33 bases downstream of the on-target adenosine.

In some embodiments, a target RNA can be an LRRK2 RNA. In this example, an engineered guide RNA comprising a barbell macro-footprint sequence, upon hybridization with the LRRK2 mRNA, forms a guide-target RNA scaffold with the LRRK2 RNA. The LRRK2 guide-target RNA scaffold when present comprises a right internal loop (e.g., a right barbell) and a left internal loop (e.g., a left barbell) manifested from the barbell macro-footprint sequence.

In some embodiments, a guide RNA targeting LRRK2 can comprise a first internal loop and a second internal loop independently positioned as follows: the first internal loop is positioned at a distance of about 2 bases or greater upstream of the on-target adenosine (e.g., 3, 4, 5, 6, 7, 8, 9, 10, 11, 12, 13, 14, 15, 16, 17, 18, 19, 20), about 20 bases or fewer upstream of the on-target adenosine (e.g., 19, 18, 17, 16, 15, 14, 13, 12, 11, 10, 9, 8, 7, 6, 5, 4, 3, 2), or from about 2 bases to about 20 bases upstream of the on-target adenosine (e.g., 3-19, 4-18, 5-17, 6-16, 7-15, 8-14, 9-13, 10-12); and the second internal loop is positioned at a distance of about 12 bases or greater downstream of the on-target adenosine (e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40), about 40 bases or fewer downstream of the on-target adenosine (e.g., 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2), from about 12 bases to about 40 bases downstream of the on-target adenosine (e.g., 13-39, 14-38, 15-37, 16-36, 17-35, 18-34, 19-33, 20-32, 21-31, 22-30, 23-29, 24-28, 25-27), or 24 bases, 26 bases, 28 bases, or 30 bases downstream of the on-target adenosine of the target RNA.

In some embodiments, a guide RNA targeting LRRK2 can comprise: a first internal loop positioned about 5 bases upstream of the on-target adenosine and a second internal loop positioned about 27 bases downstream of the on-target adenosine; a first internal loop positioned about 5 bases upstream of the on-target adenosine and a second internal loop positioned about 32 bases downstream of the on-target adenosine; a first internal loop positioned about 5 bases upstream of the on-target adenosine and a second internal loop positioned about 33 bases downstream of the on-target adenosine; a first internal loop positioned about 9 bases upstream of the on-target adenosine and a second internal loop positioned about 33 bases downstream of the on-target adenosine; a first internal loop positioned about 10 bases upstream of the on-target adenosine and a second internal loop positioned about 33 bases downstream of the on-target adenosine; a first internal loop positioned about 13 bases upstream of the on-target adenosine and a second internal loop positioned about 33 bases downstream of the on-target adenosine; a first internal loop positioned about 14 bases upstream of the on-target adenosine and a second internal loop positioned about 33 bases downstream of the on-target adenosine; or a first internal loop positioned about 15 bases upstream of the on-target adenosine and a second internal loop positioned about 33 bases downstream of the on-target adenosine.

In some embodiments, an engineered guide RNA targeting LRRK2 can comprise a first internal loop positioned at a distance of about 10 bases upstream of the on-target adenosine and a second internal loop positioned at a distance of about 34 bases downstream of the on-target adenosine.

In some embodiments, the first barbell or second barbell can be symmetrical internal loops that comprise 8 bases. In some embodiments, either the first barbell or the second barbell may not comprise 8 bases.

In some embodiments, the first barbell or second barbell can be symmetrical internal loops that comprise 6 bases. In some embodiments, either the first barbell or the second barbell may not comprise 6 bases.

### Micro-Footprint Sequences

As disclosed herein, an engineered guide RNA comprising a barbell macro-footprint sequence comprises a micro-footprint sequence in between the barbell macro-footprint that, upon hybridization to the target RNA, produces latent structures that manifest as one or more structural features.

In some embodiments, the present disclosure provides for engineered guide RNAs comprising a barbell macro-footprint. In some embodiments, the present disclosure provides for engineered guide RNAs comprising a micro-footprint. In some embodiments, the present disclosure provides for engineered guide RNAs comprising a macro-footprint and a micro-footprint, where the macro-footprint includes barbells (or internal loops) near the 5′ and 3′ ends of the guide-target RNA scaffold and the micro-footprint includes other structural features including, but not limited to, mismatches, symmetric internal loops, asymmetric internal loops, symmetric bulges, or asymmetric bulges. For example, an engineered guide RNA disclosed herein can have a macro-footprint and a micro-footprint of A/G mismatches at local off-target adenosines. An engineered guide RNA disclosed herein may have a macro-footprint and a micro-footprint of I/O asymmetric bulges (formed by an A in the target RNA and deletion of a U in the engineered guide RNA) at local off-target adenosines. An engineered guide RNA disclosed herein can have a macro-footprint of barbells (including an internal loop near the 5′ end of the guide-target RNA scaffold and an internal loop near the 3′ end of the guide-target RNA scaffold) and a micro-footprint of A/G mismatches at local off-target adenosines. An engineered guide RNA disclosed herein may have a macro-footprint of barbells (including an internal loop near the 5′ end of the guide-target RNA scaffold and an internal loop near the 3′ end of the guide-target RNA scaffold) and a micro-footprint of I/O asymmetric bulges (formed by an A in the target RNA and deletion of a U in the engineered guide RNA) at local off-target adenosines. In some embodiments, an engineered guide RNA disclosed herein may have a macro-footprint of barbells (including an internal loop near the 5′ end of the guide-target RNA scaffold and an internal loop near the 3′ end of the guide-target RNA scaffold) and a micro-footprint of a 5/5 symmetric loop, 1/1 G/G mismatch, and a 3/3 symmetric bulge to boost on-target adenosine editing while also reducing local off-target adenosine editing. In some embodiments, the barbell macro-footprint is engineered to form an internal loop at the −14 position and an internal loop at the +22 position relative to the target adenosine (position 0). In some embodiments, the barbell macro-footprint is engineered to form an internal loop at the −20 position and an internal loop at the +26 position relative to the target adenosine (position 0).

A micro-footprint sequence of a guide RNA comprising latent structures (e.g., a “latent structure guide RNA”) can comprise a portion of sequence that, upon hybridization to a target RNA, forms at least a portion of a structural feature, other than a single A/C mismatch feature at the target adenosine to be edited. A structural feature of the latent guide RNA is thus latent, in that the structural feature forms, forms only upon, or substantially forms, upon hybridization of the guide RNA to the target RNA. In some embodiments, a latent structural feature formed upon hybridization to a target RNA includes at least two contiguous nucleotides of the guide RNA. In some instances, a latent structural feature can include a mismatch that is in addition to the A/C mismatch feature at the target adenosine to be edited, with this additional mismatch providing an increase in an amount of editing of the target RNA in the presence of the RNA editing entity, relative to an otherwise comparable guide RNA lacking the additional mismatch. In some embodiments, the engineered guide RNAs disclosed herein lack an RNA editing entity recruiting domain that is formed and present in the absence of binding to the target RNA. A guide-target RNA scaffold, as disclosed herein, can be a resulting double stranded RNA duplex formed upon hybridization of a guide RNA to a target RNA, where the guide RNA prior to hybridizing to the target RNA comprise a portion of sequence that, upon hybridization to a target RNA, forms at least a portion of a structural feature, other than a single A/C mismatch feature at the target adenosine to be edited. Accordingly, a guide-target RNA scaffold has structural features formed within the double stranded RNA duplex. For example, the guide-target RNA scaffold can have two or more features selected from the group consisting of a bulge, mismatch, internal loop, hairpin, wobble base pair, and any combination thereof. In some embodiments, engineered guide RNAs with latent structure lack an RNA editing entity recruiting domain that is formed and present in the absence of binding to the target RNA. In some embodiments, engineered guide RNAs with latent structure further comprise a recruiting domain that is formed and present in the absence of binding to the target RNA.

In some examples, an engineered guide RNA disclosed herein, when present in an aqueous solution and not bound to the target RNA molecule, does not recruit an RNA editing entity. In some examples, (i) the engineered guide RNA, when present in an aqueous solution and not bound to the target RNA molecule, does not comprise any bulges, internal loops, or hairpins; (ii) the engineered guide RNA, when present in an aqueous solution and not bound to the target RNA molecule, does not comprise any bulges, internal loops, or hairpins that recruit a human ADAR1 with a dissociation constant lower than about 100 nM, 200 nM, 300 nM, 400 nM, 500 nM, 600 nM, 700 nM, 800 nM, 900 nM, or 1,000 nM as determined by an in vitro assay; (iii) the engineered guide RNA, upon at least partially binding to the target RNA molecule and thereby forming a guide-target RNA scaffold, is configured to adopt a structural feature (along with the target RNA) that recruits an RNA editing entity; or (iv) any combination thereof. In some examples, the engineered guide RNA, when present in an aqueous solution and not bound to the target RNA molecule, if it binds to the RNA editing entity, does so with a dissociation constant of about greater than or equal to about 100 nM, 200 nM, 300 nM, 400 nM, 500 nM, 600 nM, 700 nM, 800 nM, 900 nM, or 1,000 nM. In some examples, the engineered guide RNA, when present in an aqueous solution and not bound to the target RNA molecule, if it binds to the RNA editing entity, does so with a dissociation constant of about greater than or equal to about 500 nM. In some examples, the engineered guide RNAs disclosed herein, when present in an aqueous solution and not bound to the target RNA molecule, lack a structural feature described herein. In some examples, the engineered guide RNAs disclosed herein, when present in an aqueous solution and not bound to the target RNA molecule does not comprise any bulges, internal loops, or hairpins. In some examples, the engineered guide RNAs disclosed herein, when present in an aqueous solution and not bound to the target RNA molecule, can be linear and do not comprise any structural features.

In some examples, an engineered guide RNA can be configured to facilitate an editing of a base of a nucleotide or polynucleotide of a region of a target RNA by a subject RNA editing entity. In order to facilitate editing, an engineered guide RNA of the disclosure can recruit an RNA editing entity (e.g., an adenosine deaminase).

In cases where an RNA editing entity recruiting domain formed and present in the absence of binding to a target RNA is not included in an engineered guide RNA, the engineered guide RNA can be still capable of associating with a subject RNA editing entity (e.g., ADAR) to facilitate editing of a target RNA, modulate expression of a polypeptide encoded by a subject target RNA, or both. This can be achieved through the presence of structural features that manifest from latent structures formed upon hybridization of the guide RNA and target RNA. Structural features can comprise any one of a: mismatch, symmetrical bulge, asymmetrical bulge, symmetrical internal loop, asymmetrical internal loop, hairpins, wobble base pairs, a structured motif, circularized RNA, chemical modification, or any combination thereof. In an aspect, a double stranded RNA (dsRNA) substrate (e.g., a guide-target RNA scaffold), is formed upon hybridization of an engineered guide RNA of the present disclosure to a target RNA. The resulting dsRNA is also referred to herein as a “guide-target RNA scaffold.” Described herein is a feature, which corresponds to one of several structural features that can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a hairpin comprising a non-targeting domain). Engineered guide RNAs of the present disclosure can have from 1 to 50 features. For example, engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 5 to 25, from 5 to 30, from 5 to 35, from 5 to 40, from 5 to 45, from 5 to 50, from 1 to 2, from 1 to 3, from 1 to 4, from 1 to 5, from 1 to 6, from 1 to 7, from 1 to 8, from 1 to 9, from 1 to 10, from 1 to 11, from 1 to 12, from 1 to 13, from 1 to 14, from 1 to 15, from 1 to 16, from 1 to 17, from 1 to 18, from 1 to 19, from 1 to 20, from 1 to 21, from 1 to 22, from 1 to 23, from 1 to 24, from 1 to 25, from 1 to 26, from 1 to 27, from 1 to 28, from 1 to 29, from 1 to 30, from 1 to 31, from 1 to 32, from 1 to 33, from 1 to 34, from 1 to 35, from 1 to 36, from 1 to 37, from 1 to 38, from 1 to 39, from 1 to 40, from 1 to 41, from 1 to 42, from 1 to 43, from 1 to 44, from 1 to 45, from 1 to 46, from 1 to 47, from 1 to 48, from 1 to 49, from 4 to 5, from 2 to 10, from 20 to 40, from 10 to 40, from 20 to 50, from 30 to 50, from 4 to 7, or from 8 to 10 features. In some instances, an engineered guide RNA can have at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 features.

As disclosed herein, a “structured motif” comprises two or more features in a dsRNA substrate (e.g., a guide-target RNA scaffold).

As described herein, a double stranded RNA (dsRNA) substrate (e.g., a guide-target RNA scaffold) is formed upon hybridization of an engineered guide RNA of the present disclosure to a target RNA. As disclosed herein, a “mismatch” refers to a single nucleotide in a guide RNA that is unpaired to an opposing single nucleotide in a target RNA within the guide-target RNA scaffold. A mismatch can comprise any two single nucleotides that do not base pair, are not complementary, or both. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch can be an A/C mismatch as described above (e.g., an A/C mismatch comprised in the micro-footprint sequence). An A/C mismatch can comprise a C in an engineered guide RNA of the present disclosure opposite an A in a target RNA. An A/C mismatch can comprise an A in an engineered guide RNA of the present disclosure opposite an C in a target RNA. In an embodiment, a G/G mismatch can comprise a G in an engineered guide RNA of the present disclosure opposite a G in a target RNA. In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA. In some embodiments, a mismatch comprises a G/G mismatch. In further embodiments, a mismatch comprises an A/C mismatch, wherein the A can be in the target RNA and the C can be in the targeting sequence of the engineered guide RNA. In other embodiments, the A in the A/C mismatch can be the base of the nucleotide in the target RNA edited by a subject RNA editing entity.

In some examples, a structural feature is present when an engineered guide RNA is in association with a target RNA. A structural feature of an engineered guide RNA can form a substantially linear two-dimensional structure. A structural feature of an engineered guide RNA can comprise a linear region, a stem-loop, a cruciform, a toe hold, a mismatch bulge, or any combination thereof. In some instances, a structural feature can comprise a stem, a hairpin loop, a pseudoknot, a bulge, an internal loop, a multiloop, a G-quadruplex, or any combination thereof. In some examples, an engineered guide RNA can adopt an A-form, a B-form, a Z-form, or any combination thereof. In some embodiments, a linear engineered guide RNA can comprise ribozyme domain. In some embodiments, a linear engineered guide RNA may not comprise a ribozyme domain.

In some cases, a structural feature can be a hairpin. In some cases, an engineered guide RNA can lack a hairpin domain (e.g., the engineered guide RNA does not form an intramolecular hairpin in the absence of hybridization to a target RNA). In other cases, an engineered guide RNA can contain a hairpin domain or more than one hairpin domain. A hairpin can be located anywhere in a guide RNA. As disclosed herein, a “hairpin” includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and a non-base paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can be a recruitment hairpin or a non-recruitment hairpin. A hairpin can be located anywhere within the engineered guide RNAs of the present disclosure. In some embodiments, one or more hairpins can be present at the 3′ end of an engineered guide RNAs of the present disclosure, at the 5′ end of an engineered guide RNAs of the present disclosure or within the targeting sequence of an engineered guide RNAs of the present disclosure, or any combination thereof.

As disclosed herein, a hairpin can refer to a recruitment hairpin, a non-recruitment hairpin, or any combination thereof. A “recruitment hairpin,” as disclosed herein, refers to a hairpin which recruits or recruits at least in part an RNA editing entity (e.g., an ADAR). In yet another aspect, a structural feature comprises a non-recruitment hairpin. A non-recruitment hairpin, as disclosed herein, does not have a primary function of recruiting an RNA editing entity. A non-recruitment hairpin, in some instances, does not recruit an RNA editing entity. A non-recruitment hairpin can exhibit functionality that improves localization of the engineered guide RNAs to the target RNA. In some embodiments, a non-recruitment hairpin exhibits functionality that improves localization of the engineered guide RNAs to the region of the target RNA for hybridization. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be coded for in constructs comprising engineered guide RNAs. Latent structure guide RNAs can have a barbell macro-footprint sequence and at least some elements of a micro-footprint sequence. Further, pre-formed structures, such as a U7 hairpin, an smOPT sequence, or both, can be added to or bolted on to the latent structure guide RNAs to form engineered guide RNAs. Engineered guide RNAs can be expressed from constructs comprising, for example, a promoter (e.g., U1, U6, U7) and optionally operably linked to a terminator sequence (e.g., U7 terminator sequence or a U7 truncated terminator sequence). The Sm binding domain can comprise an RNA-binding domain that recognizes with high specificity, RNA sequences comprising U-rich portions. In some instances, an Sm binding domain can be responsible for both protein oligomerization and specific RNA binding.

A hairpin of the present disclosure can be of any length. In an aspect, a hairpin can be from about 10-500 or more nucleotides. In some cases, a hairpin can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides. In other cases, a hairpin can also comprise 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

In another aspect, a structural feature comprises a wobble base. A “wobble base pair” refers to two bases that weakly pair. For example, a wobble base pair of the present disclosure can refer to a G paired with a U. Thus, a wobble base pair can be a structural feature formed from latent structure provided by an engineered latent guide RNA.

In some cases, a structural feature can be a bulge. As disclosed herein, a double stranded RNA (dsRNA) substrate (e.g., guide-target RNA scaffold) is formed upon hybridization of an engineered guide RNA of the present disclosure to a target RNA. As disclosed herein, a “bulge” refers to the structure substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can have from 0 to 4 contiguous nucleotides on the guide RNA side of the guide-target RNA scaffold and 1 to 4 contiguous nucleotides on the target RNA side of the guide-target RNA scaffold or a bulge can have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. In some cases, the bulge comprising contiguous nucleotides in either the engineered guide RNA or the target RNA that are not complementary to their positional counterparts on the opposite strand is flanked on both sides with hybridized, complementary dsRNA regions. Thus, a bulge can be a structural feature formed from latent structure provided by an engineered latent guide RNA. In some cases, the bulge comprising contiguous nucleotides in either the engineered guide RNA or the target RNA that are not complementary to their positional counterparts on the opposite strand is flanked on both sides with hybridized, complementary dsRNA regions. A bulge can be located at any location of a guide RNA other than the last nucleotides of either the 5′ end or the 3′ end. In some cases, a bulge can be located from about 30 to about 70 nucleotides from a 5′ hydroxyl or the 3′ hydroxyl.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

In some embodiments, selective editing of the target A is achieved by positioning the target A between two bulges (e.g., positioned between a 5′ end bulge and a 3′ end bulge, based on the engineered guide RNA). In some embodiments, the two bulges are both symmetrical bulges. In some embodiments, the two bulges each are formed by 2 nucleotides on the engineered guide RNA side of the guide-target RNA scaffold and 2 nucleotides on the target RNA side of the guide-target RNA scaffold. In some embodiments, the two bulges each are formed by 3 nucleotides on the engineered guide RNA side of the guide-target RNA scaffold and 3 nucleotides on the target RNA side of the guide-target RNA scaffold. In some embodiments, the two bulges each are formed by 4 nucleotides on the engineered guide RNA side of the guide-target RNA scaffold and 4 nucleotides on the target RNA side of the guide-target RNA scaffold. In some embodiments, the target A is position between the two bulges, and is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201,202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides from a bulge (e.g., from a 5′ end bulge or a 3′ end bulge). In some embodiments, additional structural features are located between the bulges (e.g., between the 5′ end bulge and the 3′ end bulge). In some embodiments, a mismatch in a bulge comprises a nucleotide base for editing in the target RNA (e.g., an A/C mismatch in the bulge, wherein part of the bulge in the engineered guide RNA comprises a C mismatched to an A in the part of the bulge in the target RNA, and the A is edited).

In an aspect, a double stranded RNA (dsRNA) substrate (e.g., guide-target RNA scaffold) can be formed upon hybridization of an engineered guide RNA of the present disclosure to a target RNA. A bulge can be a symmetrical bulge or an asymmetrical bulge. A “symmetrical bulge” is formed when the same number of nucleotides is present on each side of the bulge. A symmetrical bulge can have from 2 to 4 nucleotides on the engineered guide RNA side of the guide-target RNA scaffold or the target RNA side of the guide-target RNA scaffold. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold and 2 nucleotides on the target RNA side of the guide-target RNA scaffold. A symmetrical bulge of the present disclosure can be formed by 3 nucleotides on the engineered guide RNA side of the guide-target RNA scaffold and 3 nucleotides on the target RNA side of the guide-target RNA scaffold. A symmetrical bulge of the present disclosure can be formed by 4 nucleotides on the engineered guide RNA side of the guide-target RNA scaffold and 4 nucleotides on the target RNA side of the guide-target RNA scaffold. Thus, a symmetrical bulge can be a structural feature formed from latent structure provided by an engineered latent guide RNA.

In some cases, a double stranded RNA (dsRNA) substrate (e.g., guide-target RNA scaffold) can be formed upon hybridization of an engineered guide RNA of the present disclosure to a target RNA. A bulge can be a symmetrical bulge or an asymmetrical bulge. An “asymmetrical bulge” is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. An asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

In an aspect, a double stranded RNA (dsRNA) substrate (e.g., guide-target RNA scaffold) can be formed upon hybridization of an engineered guide RNA of the present disclosure to a target RNA. An internal loop can be a symmetrical internal loop or an asymmetrical internal loop. As disclosed herein, an “internal loop” refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can help with base flipping of the target A in the target RNA to be edited.

In some embodiments, selective editing of the target A is achieved by positioning the target A between two loops (e.g., positioned between a 5′ end loop and a 3′ end loop, based on the engineered guide RNA). In some embodiments, one side of the internal loop, either on the target RNA side or the engineered polynucleotide side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1000 nucleotides. Thus, an internal loop can be a structural feature formed from latent structure provided by an engineered latent guide RNA.

As described herein, a double stranded RNA (dsRNA) substrate (e.g., a guide-target RNA scaffold) is formed upon hybridization of an engineered guide RNA of the present disclosure to a target RNA. An internal loop can be a symmetrical internal loop or an asymmetrical internal loop. A “symmetrical internal loop” is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 15 nucleotides on the engineered polynucleotide side of the guide-target RNA scaffold target and 15 nucleotides on the target RNA side of the guide-target RNA scaffold. A symmetrical internal loop of the present disclosure can be formed by 20 nucleotides on the engineered polynucleotide side of the guide-target RNA scaffold target and 20 nucleotides on the target RNA side of the guide-target RNA scaffold. A symmetrical internal loop of the present disclosure can be formed by 30 nucleotides on the engineered polynucleotide side of the guide-target RNA scaffold target and 30 nucleotides on the target RNA side of the guide-target RNA scaffold. A symmetrical internal loop of the present disclosure can be formed by 40 nucleotides on the engineered polynucleotide side of the guide-target RNA scaffold target and 40 nucleotides on the target RNA side of the guide-target RNA scaffold. A symmetrical internal loop of the present disclosure can be formed by 50 nucleotides on the engineered polynucleotide side of the guide-target RNA scaffold target and 50 nucleotides on the target RNA side of the guide-target RNA scaffold. A symmetrical internal loop of the present disclosure can be formed by 60 nucleotides on the engineered polynucleotide side of the guide-target RNA scaffold target and 60 nucleotides on the target RNA side of the guide-target RNA scaffold. A symmetrical internal loop of the present disclosure can be formed by 70 nucleotides on the engineered polynucleotide side of the guide-target RNA scaffold target and 70 nucleotides on the target RNA side of the guide-target RNA scaffold. A symmetrical internal loop of the present disclosure can be formed by 80 nucleotides on the engineered polynucleotide side of the guide-target RNA scaffold target and 80 nucleotides on the target RNA side of the guide-target RNA scaffold. A symmetrical internal loop of the present disclosure can be formed by 90 nucleotides on the engineered polynucleotide side of the guide-target RNA scaffold target and 90 nucleotides on the target RNA side of the guide-target RNA scaffold. A symmetrical internal loop of the present disclosure can be formed by 100 nucleotides on the engineered polynucleotide side of the guide-target RNA scaffold target and 100 nucleotides on the target RNA side of the guide-target RNA scaffold. A symmetrical internal loop of the present disclosure can be formed by 110 nucleotides on the engineered polynucleotide side of the guide-target RNA scaffold target and 110 nucleotides on the target RNA side of the guide-target RNA scaffold. A symmetrical internal loop of the present disclosure can be formed by 120 nucleotides on the engineered polynucleotide side of the guide-target RNA scaffold target and 120 nucleotides on the target RNA side of the guide-target RNA scaffold. A symmetrical internal loop of the present disclosure can be formed by 130 nucleotides on the engineered polynucleotide side of the guide-target RNA scaffold target and 130 nucleotides on the target RNA side of the guide-target RNA scaffold. A symmetrical internal loop of the present disclosure can be formed by 140 nucleotides on the engineered polynucleotide side of the guide-target RNA scaffold target and 140 nucleotides on the target RNA side of the guide-target RNA scaffold. A symmetrical internal loop of the present disclosure can be formed by 150 nucleotides on the engineered polynucleotide side of the guide-target RNA scaffold target and 150 nucleotides on the target RNA side of the guide-target RNA scaffold. A symmetrical internal loop of the present disclosure can be formed by 200 nucleotides on the engineered polynucleotide side of the guide-target RNA scaffold target and 200 nucleotides on the target RNA side of the guide-target RNA scaffold. A symmetrical internal loop of the present disclosure can be formed by 250 nucleotides on the engineered polynucleotide side of the guide-target RNA scaffold target and 250 nucleotides on the target RNA side of the guide-target RNA scaffold. A symmetrical internal loop of the present disclosure can be formed by 300 nucleotides on the engineered polynucleotide side of the guide-target RNA scaffold target and 300 nucleotides on the target RNA side of the guide-target RNA scaffold. A symmetrical internal loop of the present disclosure can be formed by 350 nucleotides on the engineered polynucleotide side of the guide-target RNA scaffold target and 350 nucleotides on the target RNA side of the guide-target RNA scaffold. A symmetrical internal loop of the present disclosure can be formed by 400 nucleotides on the engineered polynucleotide side of the guide-target RNA scaffold target and 400 nucleotides on the target RNA side of the guide-target RNA scaffold. A symmetrical internal loop of the present disclosure can be formed by 450 nucleotides on the engineered polynucleotide side of the guide-target RNA scaffold target and 450 nucleotides on the target RNA side of the guide-target RNA scaffold. A symmetrical internal loop of the present disclosure can be formed by 500 nucleotides on the engineered polynucleotide side of the guide-target RNA scaffold target and 500 nucleotides on the target RNA side of the guide-target RNA scaffold. A symmetrical internal loop of the present disclosure can be formed by 600 nucleotides on the engineered polynucleotide side of the guide-target RNA scaffold target and 600 nucleotides on the target RNA side of the guide-target RNA scaffold. A symmetrical internal loop of the present disclosure can be formed by 700 nucleotides on the engineered polynucleotide side of the guide-target RNA scaffold target and 700 nucleotides on the target RNA side of the guide-target RNA scaffold. A symmetrical internal loop of the present disclosure can be formed by 800 nucleotides on the engineered polynucleotide side of the guide-target RNA scaffold target and 800 nucleotides on the target RNA side of the guide-target RNA scaffold. A symmetrical internal loop of the present disclosure can be formed by 900 nucleotides on the engineered polynucleotide side of the guide-target RNA scaffold target and 900 nucleotides on the target RNA side of the guide-target RNA scaffold. A symmetrical internal loop of the present disclosure can be formed by 1000 nucleotides on the engineered polynucleotide side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

In some embodiments, the target A is positioned between the two loops, and is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides from a loop (e.g., from a 5′ end loop or a 3′ end loop). In some embodiments, additional structural features are located between the loops (e.g., between the 5′ end loop and the 3′ end loop). In some embodiments, a mismatch in a loop comprises a nucleotide base for editing in the target RNA (e.g., an A/C mismatch in the loop, wherein part of the loop in the engineered guide RNA comprises a C mismatched to an A in the part of the loop in the target RNA, and the A is edited).

As disclosed herein, a double-stranded RNA (dsRNA) substrate (e.g., a guide-target RNA scaffold) is formed upon hybridization of an engineered guide RNA of the present disclosure to a target RNA. An internal loop can be a symmetrical internal loop or an asymmetrical internal loop. An “asymmetrical internal loop” is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered polynucleotide side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered polynucleotide side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered polynucleotide side of the guide-target RNA scaffold. An asymmetrical internal loop of the present disclosure can be formed by 5 nucleotides on the target RNA side of the guide-target RNA scaffold and 100 nucleotides on the engineered polynucleotide side of the guide-target RNA scaffold. An asymmetrical internal loop of the present disclosure can be formed by 5 nucleotides on the target RNA side of the guide-target RNA scaffold and 150 nucleotides on the engineered polynucleotide side of the guide-target RNA scaffold. An asymmetrical internal loop of the present disclosure can be formed by 5 nucleotides on the target RNA side of the guide-target RNA scaffold and 200 nucleotides on the engineered polynucleotide side of the guide-target RNA scaffold. An asymmetrical internal loop of the present disclosure can be formed by 5 nucleotides on the target RNA side of the guide-target RNA scaffold and 300 nucleotides on the engineered polynucleotide side of the guide-target RNA scaffold. An asymmetrical internal loop of the present disclosure can be formed by 5 nucleotides on the target RNA side of the guide-target RNA scaffold and 400 nucleotides on the engineered polynucleotide side of the guide-target RNA scaffold. An asymmetrical internal loop of the present disclosure can be formed by 5 nucleotides on the target RNA side of the guide-target RNA scaffold and 500 nucleotides on the engineered polynucleotide side of the guide-target RNA scaffold. An asymmetrical internal loop of the present disclosure can be formed by 5 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 5 nucleotides on the engineered polynucleotide side of the guide-target RNA scaffold. An asymmetrical internal loop of the present disclosure can be formed by 500 nucleotides on the target RNA side of the guide-target RNA scaffold and 5 nucleotides on the engineered polynucleotide side of the guide-target RNA scaffold. An asymmetrical internal loop of the present disclosure can be formed by 400 nucleotides on the target RNA side of the guide-target RNA scaffold and 5 nucleotides on the engineered polynucleotide side of the guide-target RNA scaffold. An asymmetrical internal loop of the present disclosure can be formed by 300 nucleotides on the target RNA side of the guide-target RNA scaffold and 5 nucleotides on the engineered polynucleotide side of the guide-target RNA scaffold. An asymmetrical internal loop of the present disclosure can be formed by 200 nucleotides on the target RNA side of the guide-target RNA scaffold and 5 nucleotides on the engineered polynucleotide side of the guide-target RNA scaffold. An asymmetrical internal loop of the present disclosure can be formed by 150 nucleotides on the target RNA side of the guide-target RNA scaffold and 5 nucleotides on the engineered polynucleotide side of the guide-target RNA scaffold. An asymmetrical internal loop of the present disclosure can be formed by 100 nucleotides on the target RNA side of the guide-target RNA scaffold and 5 nucleotides on the engineered polynucleotide side of the guide-target RNA scaffold. An asymmetrical internal loop of the present disclosure can be formed by 50 nucleotides on the target RNA side of the guide-target RNA scaffold and 5 nucleotides on the engineered polynucleotide side of the guide-target RNA scaffold. An asymmetrical internal loop of the present disclosure can be formed by 50 nucleotides on the target RNA side of the guide-target RNA scaffold and 100 nucleotides on the engineered polynucleotide side of the guide-target RNA scaffold. An asymmetrical internal loop of the present disclosure can be formed by 50 nucleotides on the target RNA side of the guide-target RNA scaffold and 150 nucleotides on the engineered polynucleotide side of the guide-target RNA scaffold. An asymmetrical internal loop of the present disclosure can be formed by 50 nucleotides on the target RNA side of the guide-target RNA scaffold and 200 nucleotides on the engineered polynucleotide side of the guide-target RNA scaffold. An asymmetrical internal loop of the present disclosure can be formed by 50 nucleotides on the target RNA side of the guide-target RNA scaffold and 300 nucleotides on the engineered polynucleotide side of the guide-target RNA scaffold. An asymmetrical internal loop of the present disclosure can be formed by 50 nucleotides on the target RNA side of the guide-target RNA scaffold and 400 nucleotides on the engineered polynucleotide side of the guide-target RNA scaffold. An asymmetrical internal loop of the present disclosure can be formed by 50 nucleotides on the target RNA side of the guide-target RNA scaffold and 500 nucleotides on the engineered polynucleotide side of the guide-target RNA scaffold. An asymmetrical internal loop of the present disclosure can be formed by 50 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 50 nucleotides on the engineered polynucleotide side of the guide-target RNA scaffold. An asymmetrical internal loop of the present disclosure can be formed by 500 nucleotides on the target RNA side of the guide-target RNA scaffold and 50 nucleotides on the engineered polynucleotide side of the guide-target RNA scaffold. An asymmetrical internal loop of the present disclosure can be formed by 400 nucleotides on the target RNA side of the guide-target RNA scaffold and 50 nucleotides on the engineered polynucleotide side of the guide-target RNA scaffold. An asymmetrical internal loop of the present disclosure can be formed by 300 nucleotides on the target RNA side of the guide-target RNA scaffold and 50 nucleotides on the engineered polynucleotide side of the guide-target RNA scaffold. An asymmetrical internal loop of the present disclosure can be formed by 200 nucleotides on the target RNA side of the guide-target RNA scaffold and 50 nucleotides on the engineered polynucleotide side of the guide-target RNA scaffold. An asymmetrical internal loop of the present disclosure can be formed by 150 nucleotides on the target RNA side of the guide-target RNA scaffold and 50 nucleotides on the engineered polynucleotide side of the guide-target RNA scaffold. An asymmetrical internal loop of the present disclosure can be formed by 100 nucleotides on the target RNA side of the guide-target RNA scaffold and 50 nucleotides on the engineered polynucleotide side of the guide-target RNA scaffold. An asymmetrical internal loop of the present disclosure can be formed by 100 nucleotides on the target RNA side of the guide-target RNA scaffold and 150 nucleotides on the engineered polynucleotide side of the guide-target RNA scaffold. An asymmetrical internal loop of the present disclosure can be formed by 100 nucleotides on the target RNA side of the guide-target RNA scaffold and 200 nucleotides on the engineered polynucleotide side of the guide-target RNA scaffold. An asymmetrical internal loop of the present disclosure can be formed by 100 nucleotides on the target RNA side of the guide-target RNA scaffold and 300 nucleotides on the engineered polynucleotide side of the guide-target RNA scaffold. An asymmetrical internal loop of the present disclosure can be formed by 100 nucleotides on the target RNA side of the guide-target RNA scaffold and 400 nucleotides on the engineered polynucleotide side of the guide-target RNA scaffold. An asymmetrical internal loop of the present disclosure can be formed by 100 nucleotides on the target RNA side of the guide-target RNA scaffold and 500 nucleotides on the engineered polynucleotide side of the guide-target RNA scaffold. An asymmetrical internal loop of the present disclosure can be formed by 100 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 100 nucleotides on the engineered polynucleotide side of the guide-target RNA scaffold. An asymmetrical internal loop of the present disclosure can be formed by 500 nucleotides on the target RNA side of the guide-target RNA scaffold and 100 nucleotides on the engineered polynucleotide side of the guide-target RNA scaffold. An asymmetrical internal loop of the present disclosure can be formed by 400 nucleotides on the target RNA side of the guide-target RNA scaffold and 100 nucleotides on the engineered polynucleotide side of the guide-target RNA scaffold. An asymmetrical internal loop of the present disclosure can be formed by 300 nucleotides on the target RNA side of the guide-target RNA scaffold and 100 nucleotides on the engineered polynucleotide side of the guide-target RNA scaffold. An asymmetrical internal loop of the present disclosure can be formed by 200 nucleotides on the target RNA side of the guide-target RNA scaffold and 100 nucleotides on the engineered polynucleotide side of the guide-target RNA scaffold. An asymmetrical internal loop of the present disclosure can be formed by 150 nucleotides on the target RNA side of the guide-target RNA scaffold and 100 nucleotides on the engineered polynucleotide side of the guide-target RNA scaffold. An asymmetrical internal loop of the present disclosure can be formed by 150 nucleotides on the target RNA side of the guide-target RNA scaffold and 200 nucleotides on the engineered polynucleotide side of the guide-target RNA scaffold. An asymmetrical internal loop of the present disclosure can be formed by 150 nucleotides on the target RNA side of the guide-target RNA scaffold and 300 nucleotides on the engineered polynucleotide side of the guide-target RNA scaffold. An asymmetrical internal loop of the present disclosure can be formed by 150 nucleotides on the target RNA side of the guide-target RNA scaffold and 400 nucleotides on the engineered polynucleotide side of the guide-target RNA scaffold. An asymmetrical internal loop of the present disclosure can be formed by 150 nucleotides on the target RNA side of the guide-target RNA scaffold and 500 nucleotides on the engineered polynucleotide side of the guide-target RNA scaffold. An asymmetrical internal loop of the present disclosure can be formed by 150 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 150 nucleotides on the engineered polynucleotide side of the guide-target RNA scaffold. An asymmetrical internal loop of the present disclosure can be formed by 500 nucleotides on the target RNA side of the guide-target RNA scaffold and 5 nucleotides on the engineered polynucleotide side of the guide-target RNA scaffold. An asymmetrical internal loop of the present disclosure can be formed by 400 nucleotides on the target RNA side of the guide-target RNA scaffold and 150 nucleotides on the engineered polynucleotide side of the guide-target RNA scaffold. An asymmetrical internal loop of the present disclosure can be formed by 300 nucleotides on the target RNA side of the guide-target RNA scaffold and 150 nucleotides on the engineered polynucleotide side of the guide-target RNA scaffold. An asymmetrical internal loop of the present disclosure can be formed by 200 nucleotides on the target RNA side of the guide-target RNA scaffold and 300 nucleotides on the engineered polynucleotide side of the guide-target RNA scaffold. An asymmetrical internal loop of the present disclosure can be formed by 200 nucleotides on the target RNA side of the guide-target RNA scaffold and 400 nucleotides on the engineered polynucleotide side of the guide-target RNA scaffold. An asymmetrical internal loop of the present disclosure can be formed by 200 nucleotides on the target RNA side of the guide-target RNA scaffold and 500 nucleotides on the engineered polynucleotide side of the guide-target RNA scaffold. An asymmetrical internal loop of the present disclosure can be formed by 200 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 200 nucleotides on the engineered polynucleotide side of the guide-target RNA scaffold. An asymmetrical internal loop of the present disclosure can be formed by 500 nucleotides on the target RNA side of the guide-target RNA scaffold and 200 nucleotides on the engineered polynucleotide side of the guide-target RNA scaffold. An asymmetrical internal loop of the present disclosure can be formed by 400 nucleotides on the target RNA side of the guide-target RNA scaffold and 200 nucleotides on the engineered polynucleotide side of the guide-target RNA scaffold. An asymmetrical internal loop of the present disclosure can be formed by 300 nucleotides on the target RNA side of the guide-target RNA scaffold and 200 nucleotides on the engineered polynucleotide side of the guide-target RNA scaffold. An asymmetrical internal loop of the present disclosure can be formed by 300 nucleotides on the target RNA side of the guide-target RNA scaffold and 400 nucleotides on the engineered polynucleotide side of the guide-target RNA scaffold. An asymmetrical internal loop of the present disclosure can be formed by 300 nucleotides on the target RNA side of the guide-target RNA scaffold and 500 nucleotides on the engineered polynucleotide side of the guide-target RNA scaffold. An asymmetrical internal loop of the present disclosure can be formed by 300 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 300 nucleotides on the engineered polynucleotide side of the guide-target RNA scaffold. An asymmetrical internal loop of the present disclosure can be formed by 500 nucleotides on the target RNA side of the guide-target RNA scaffold and 300 nucleotides on the engineered polynucleotide side of the guide-target RNA scaffold. An asymmetrical internal loop of the present disclosure can be formed by 400 nucleotides on the target RNA side of the guide-target RNA scaffold and 300 nucleotides on the engineered polynucleotide side of the guide-target RNA scaffold. An asymmetrical internal loop of the present disclosure can be formed by 400 nucleotides on the target RNA side of the guide-target RNA scaffold and 500 nucleotides on the engineered polynucleotide side of the guide-target RNA scaffold. An asymmetrical internal loop of the present disclosure can be formed by 400 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 400 nucleotides on the engineered polynucleotide side of the guide-target RNA scaffold. An asymmetrical internal loop of the present disclosure can be formed by 500 nucleotides on the target RNA side of the guide-target RNA scaffold and 400 nucleotides on the engineered polynucleotide side of the guide-target RNA scaffold. An asymmetrical internal loop of the present disclosure can be formed by 500 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 500 nucleotides on the engineered polynucleotide side of the guide-target RNA scaffold. Thus, an asymmetrical internal loop can be a structural feature formed from latent structure provided by an engineered latent guide RNA.

Structural features that comprise a loop can be of any size 5 bases or greater. In some cases, a loop comprises at least: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 600, 700, 800, 900, or 1000 bases. In some cases, a loop comprises at least about 5-10, 5-15, 10-20, 15-25, 20-30, 5-30, 5-40, 5-50, 5-60, 5-70, 5-80, 5-90, 5-100, 5-110, 5-120, 5-130, 5-140, 5-150, 5-200, 5-250, 5-300, 5-350, 5-400, 5-450, 5-500, 5-600, 5-700, 5-800, 5-900, 5-1000, 20-50, 20-60, 20-70, 20-80, 20-90, 20-100, 20-110, 20-120, 20-130, 20-140, 20-150, 30-40, 30-50, 30-60, 30-70, 30-80, 30-90, 30-100, 30-110, 30-120, 30-130, 30-140, 30-150, 30-200, 30-250, 30-300, 30-350, 30-400, 30-450, 30-500, 30-600, 30-700, 30-800, 30-900, 30-1000, 40-50, 40-60, 40-70, 40-80, 40-90, 40-100, 40-110, 40-120, 40-130, 40-140, 40-150, 40-200, 40-250, 40-300, 40-350, 40-400, 40-450, 40-500, 40-600, 40-700, 40-800, 40-900, 40-1000, 50-60, 50-70, 50-80, 50-90, 50-100, 50-110, 50-120, 50-130, 50-140, 50-150, 50-200, 50-250, 50-300, 50-350, 50-400, 50-450, 50-500, 50-600, 50-700, 50-800, 50-900, 50-1000, 60-70, 60-80, 60-90, 60-100, 60-110, 60-120, 60-130, 60-140, 60-150, 60-200, 60-250, 60-300, 60-350, 60-400, 60-450, 60-500, 60-600, 60-700, 60-800, 60-900, 60-1000, 70-80, 70-90, 70-100, 70-110, 70-120, 70-130, 70-140, 70-150, 70-200, 70-250, 70-300, 70-350, 70-400, 70-450, 70-500, 70-600, 70-700, 70-800, 70-900, 70-1000, 80-90, 80-100, 80-110, 80-120, 80-130, 80-140, 80-150, 80-200, 80-250, 80-300, 80-350, 80-400, 80-450, 80-500, 80-600, 80-700, 80-800, 80-900, 80-1000, 90-100, 90-110, 90-120, 90-130, 90-140, 90-150, 90-200, 90-250, 90-300, 90-350, 90-400, 90-450, 90-500, 90-600, 90-700, 90-800, 90-900, 90-1000, 100-110, 100-120, 100-130, 100-140, 100-150, 100-200, 100-250, 100-300, 100-350, 100-400, 100-450, 100-500, 100-600, 100-700, 100-800, 100-900, 100-1000, 110-120, 110-130, 110-140, 110-150, 110-200, 110-250, 110-300, 110-350, 110-400, 110-450, 110-500, 110-600, 110-700, 110-800, 110-900, 110-1000, 120-130, 120-140, 120-150, 120-200, 120-250, 120-300, 120-350, 120-400, 120-450, 120-500, 120-600, 120-700, 120-800, 120-900, 120-1000, 130-140, 130-150, 130-200, 130-250, 130-300, 130-350, 130-400, 130-450, 130-500, 130-600, 130-700, 130-800, 130-900, 130-1000, 140-150, 140-200, 140-250, 140-300, 140-350, 140-400, 140-450, 140-500, 140-600, 140-700, 140-800, 140-900, 140-1000, 150-200, 150-250, 150-300, 150-350, 150-400, 150-450, 150-500, 150-600, 150-700, 150-800, 150-900, 150-1000, 200-250, 200-300, 200-350, 200-400, 200-450, 200-500, 200-600, 200-700, 200-800, 200-900, 200-1000, 250-300, 250-350, 250-400, 250-450, 250-500, 250-600, 250-700, 250-800, 250-900, 250-1000, 300-350, 300-400, 300-450, 300-500, 300-600, 300-700, 300-800, 300-900, 300-1000, 350-400, 350-450, 350-500, 350-600, 350-700, 350-800, 350-900, 350-1000, 400-450, 400-500, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, or 900-1000 bases in total.

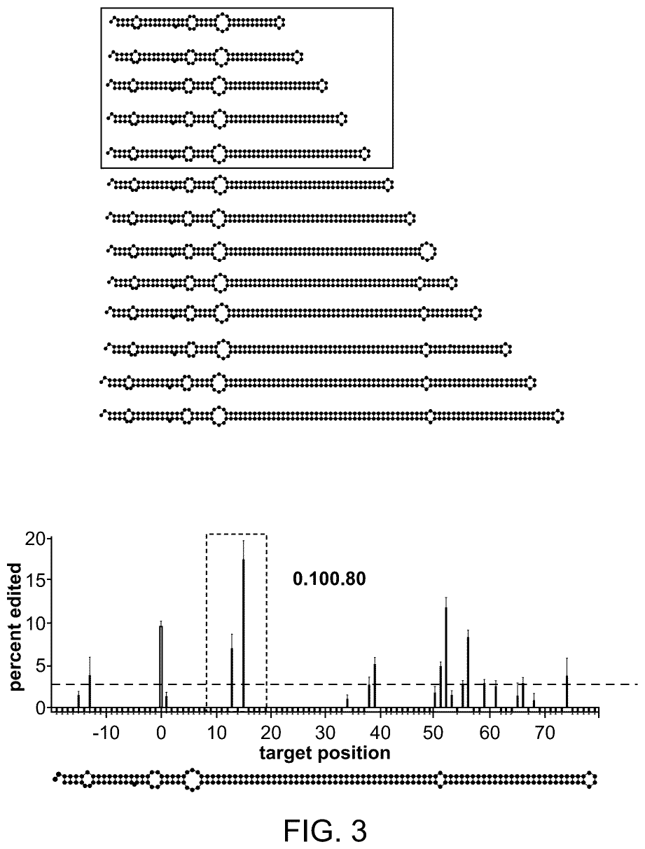

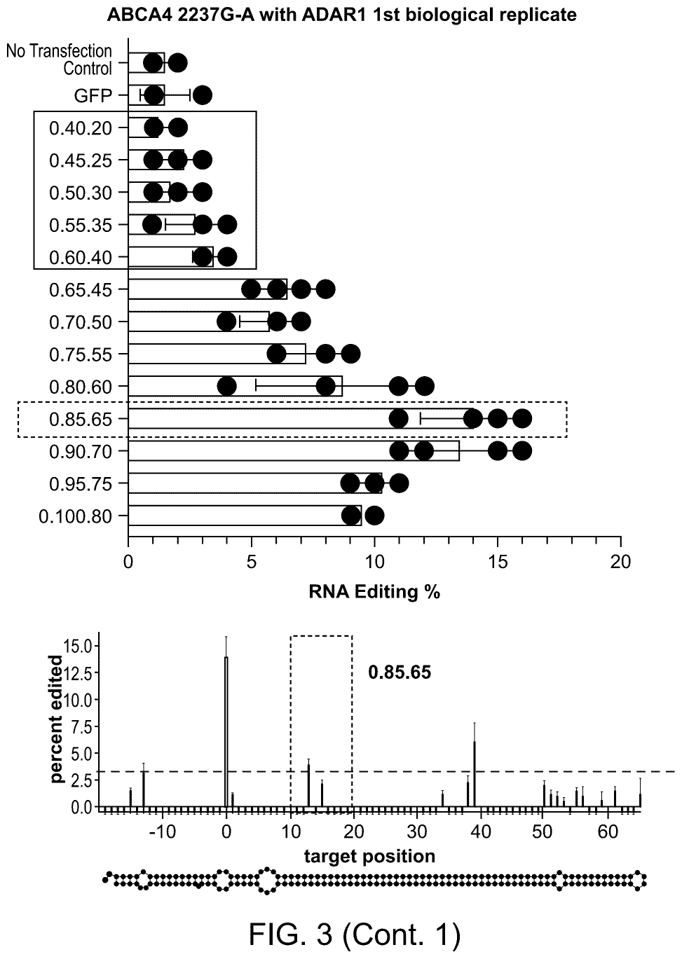

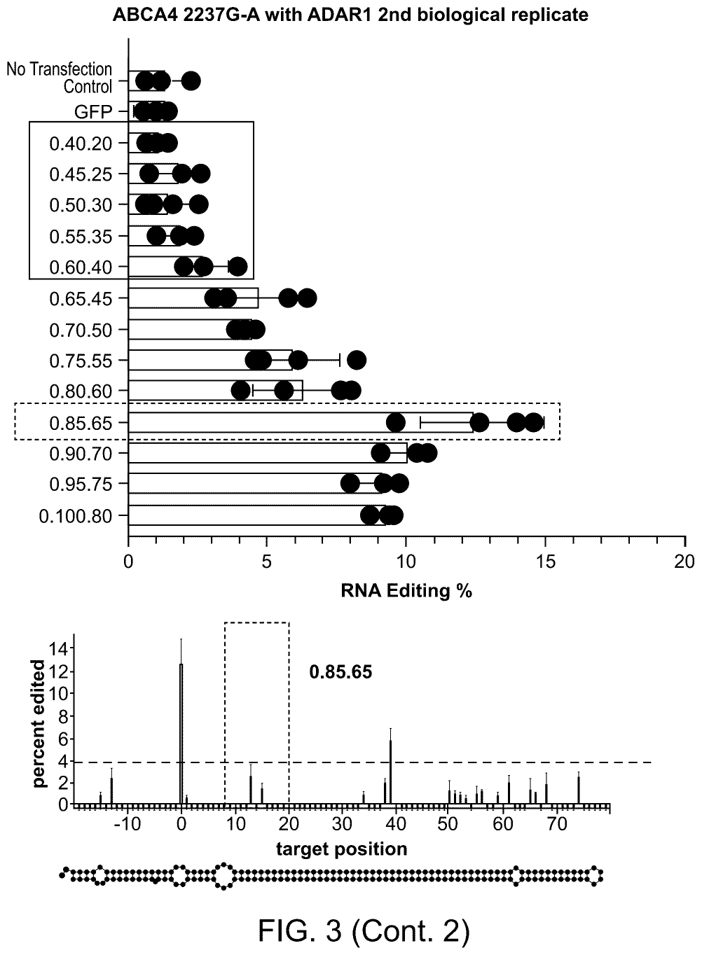

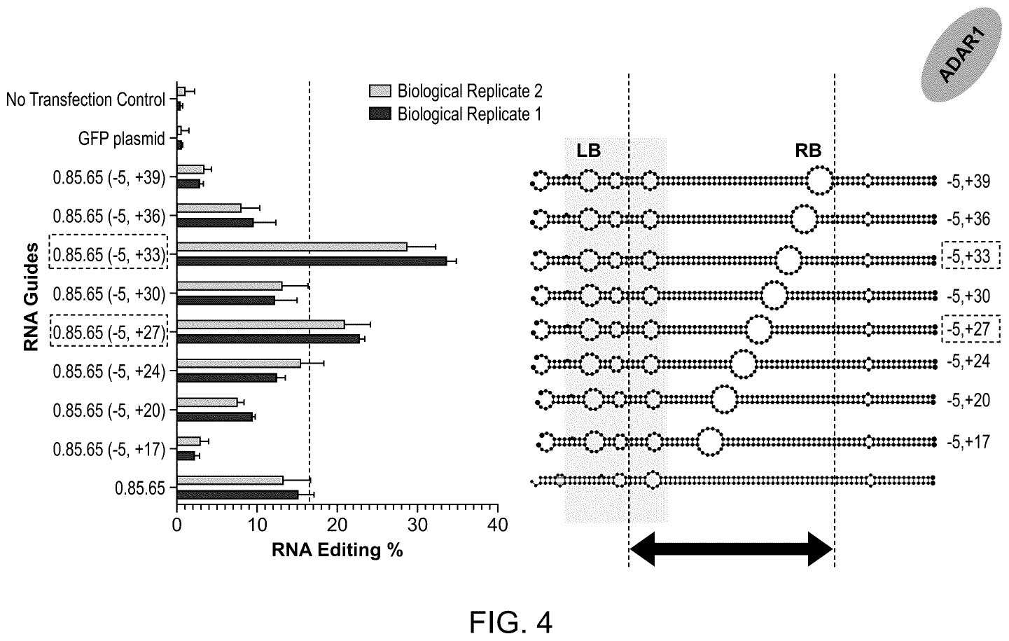

In some examples, a double stranded RNA (dsRNA) substrate (e.g., a guide-target RNA scaffold) is formed upon hybridization of an engineered guide of the present disclosure to a target RNA. In some examples, the guide-target RNA scaffold comprises structural features mimicking the structural features of a naturally occurring ADAR substrate. In some examples, the naturally occurring ADAR substrate can be a Drosophila ADAR substrate. In some examples, the naturally occurring Drosophila ADAR substrate can be as depicted in FIGS. 3 and 4 and comprises two bulges. The specific nucleotide interactions forming the structural features of the Drosophila substrate are annotated on the sequences listed in FIG. 4 and include (1) an A to C mismatch, (2) a G mismatch of a 5′ G, (3) two wobble base pairs, (4) a mismatch at the −7 position and an asymmetrical bulge at the +11 position (2/1—target/guide), and (5) an asymmetrical bulge at the +6 position (1/0—target/guide). In some examples, the structural features of the guide-target RNA scaffold mimic the structural features of a Drosophila substrate in that the double stranded substrate comprises one or more (e.g., 1, 2, 3, 4, 5, 6 or 7) of the structural features also present in the Drosophila substrate. In some examples, the one or more structural features in the guide-target RNA scaffold share at least 70%, 80%, 85%, 90%, 95%, 98%, 99% or 100% sequence homology, length with one or more (e.g., 1, 2, 3, 4, 5, 6, or 7) structural features, or both of the naturally occurring Drosophila substrate. In some examples, the one or more structural features in the double stranded substrate share no sequence homology or less than 50% sequence homology with one or more structural features of the Drosophila substrate. In some examples, the one or more features in the double stranded substrate can be positioned (relative to each other) the same or similarly as the structural features of the natural ADAR substrate.

In some cases, a structural feature can be a structured motif. As disclosed herein, a “structured motif” comprises two or more structural features in a guide-target RNA scaffold. A structured motif can comprise any combination of structural features, such as described herein, to generate an ideal substrate for ADAR editing at a precise location(s). These structured motifs could be artificially engineered to maximized ADAR editing, can be modeled to recapitulate known ADAR substrates, or both.

### Targeting Domains

Engineered guide RNAs disclosed herein comprising a barbell macro-footprint sequence and at least some elements of a micro-footprint sequence can be engineered in any way suitable for RNA editing. In some examples, a micro-footprint sequence and a macro-footprint sequence as described herein can be included as part of a targeting sequence that allows the engineered guide RNA to hybridize to a region of a target RNA molecule. A targeting sequence can also be referred to as a “targeting domain” or a “targeting region”.

In some cases, a targeting sequence of an engineered guide RNA allows the engineered guide RNA to target an RNA sequence through base pairing, such as Watson Crick base pairing. In some examples, the targeting sequence can be located at either the N-terminus or C-terminus of the engineered guide RNA. In some cases, the targeting sequence can be located at both termini. The targeting sequence can be of any length. In some cases, the targeting sequence can be at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In some cases, the targeting sequence can be no greater than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 200 nucleotides in length. In some examples, an engineered guide RNA comprises a targeting sequence that can be about 75-100, 80-110, 90-120, or 95-115 nucleotides in length. In some examples, an engineered guide comprises a targeting sequence that can be about 100 nucleotides in length.

In some examples, the target RNA sequence can be an mRNA molecule. In some examples, the mRNA molecule comprises a premature stop codon. In some examples, the mRNA comprises 1, 2, 3, 4 or 5 premature stop codons. In some examples, the stop codon can be an amber stop codon (UAG), an ochre stop codon (UAA), or an opal stop codon (UGA), or a combination thereof. In some examples, the premature stop codon can be a consequence of a point mutation. In some examples, the premature stop codon causes translation termination of an expression product expressed by the mRNA molecule. In some examples, the premature stop codon can be produced by a point mutation on an mRNA molecule in combination with two additional nucleotides. In some examples, the two additional nucleotides can be (i) a U and (ii) an A or a G, on a 5′ and a 3′ end of the point mutation.

In some examples, the target RNA sequence can be a pre-mRNA molecule. In some examples, the pre-mRNA molecule comprises a splice site mutation. In some examples, the splice site mutation facilitates unintended splicing of a pre-mRNA molecule. In some examples, the splice site mutation results in mistranslation, truncation, or both mistranslation and truncation of a protein encoded by the pre-mRNA molecule.

In some examples, the target RNA molecule can be a pre-mRNA or mRNA molecule encoded by an ABCA4, APP, SERPINA1, HEXA, LRRK2, SNCA, CFTR, APP, GBA, PINK1 or LIPA gene, a fragment of any of these, or any combination thereof. In some examples, the target RNA molecule encodes an ABCA4, APP, SERPINA1, HEXA, LRRK2, SNCA, CFTR, APP, GBA, PINK1, Tau, or LIPA protein, a fragment of any of these, or a combination thereof. In some examples, the DNA encoding the RNA molecule comprises a mutation relative to an otherwise identical reference DNA molecule. In some examples, the RNA molecule comprises a mutation relative to an otherwise identical reference RNA molecule. In some examples, the protein encoded for by the target RNA molecule comprises a mutation relative to an otherwise identical reference protein.

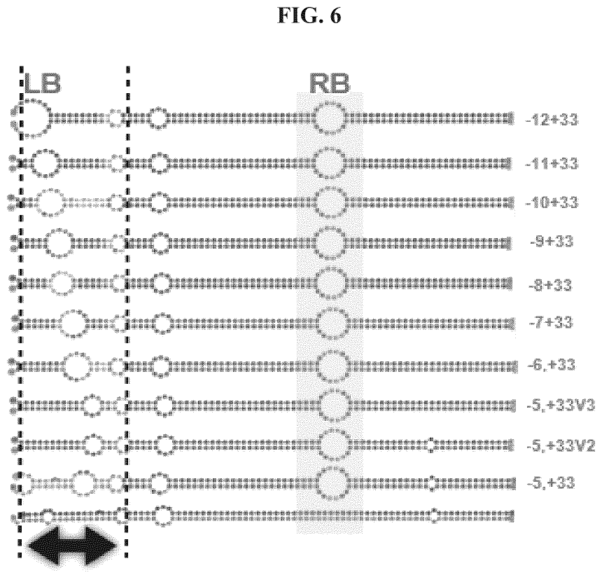

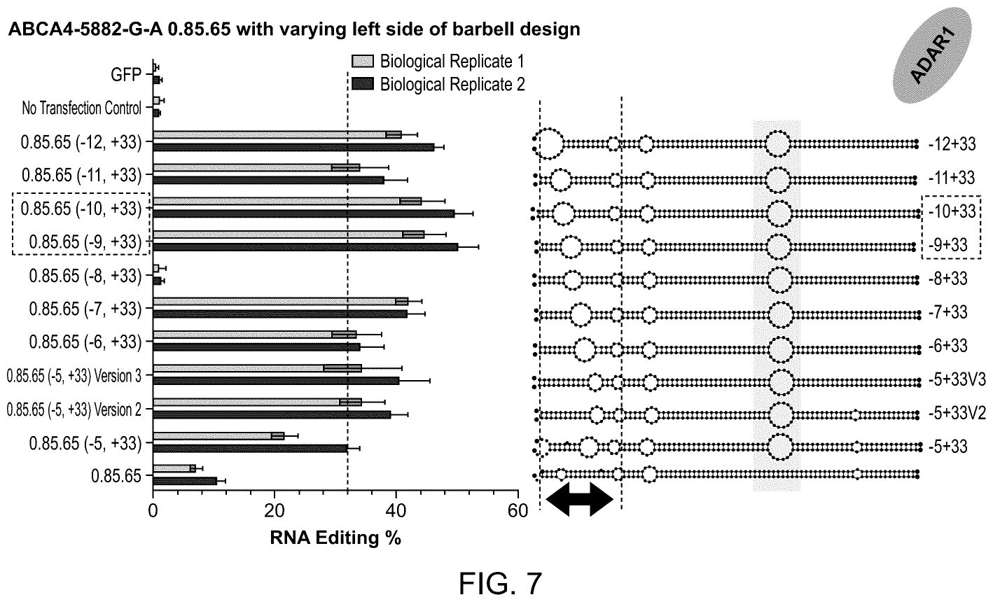

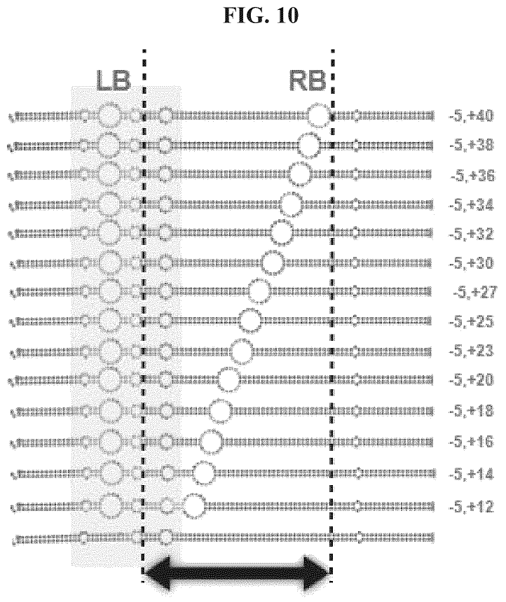

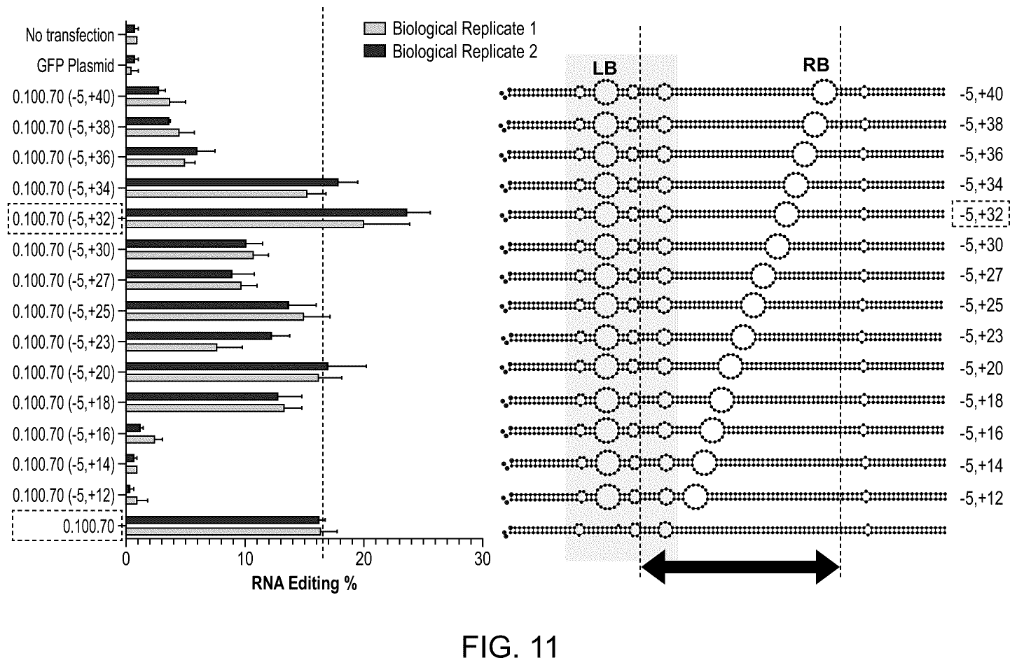

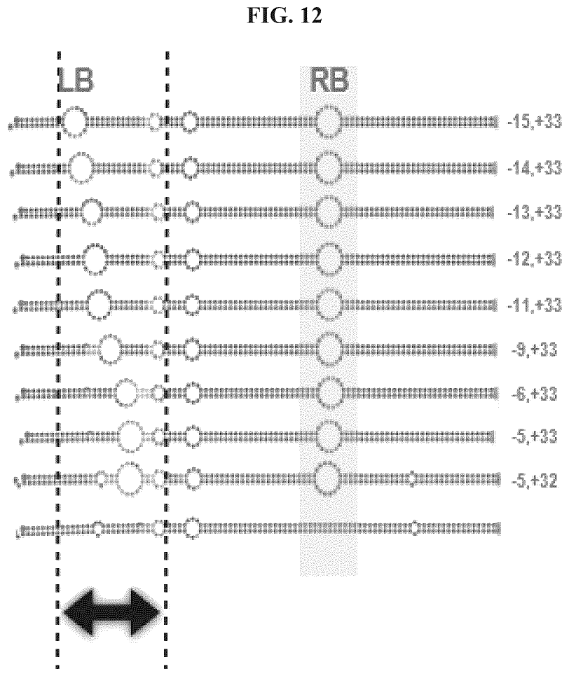

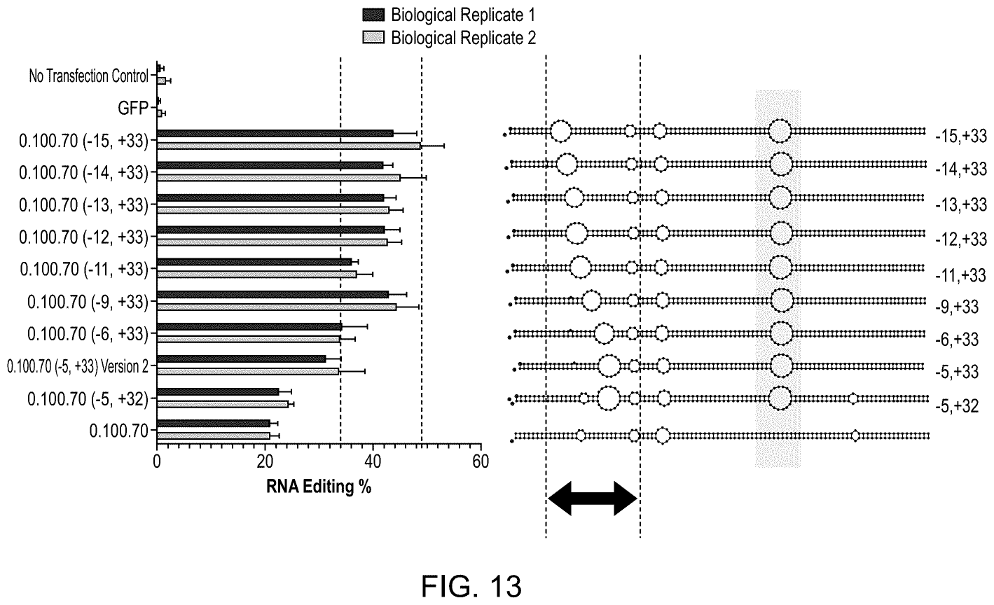

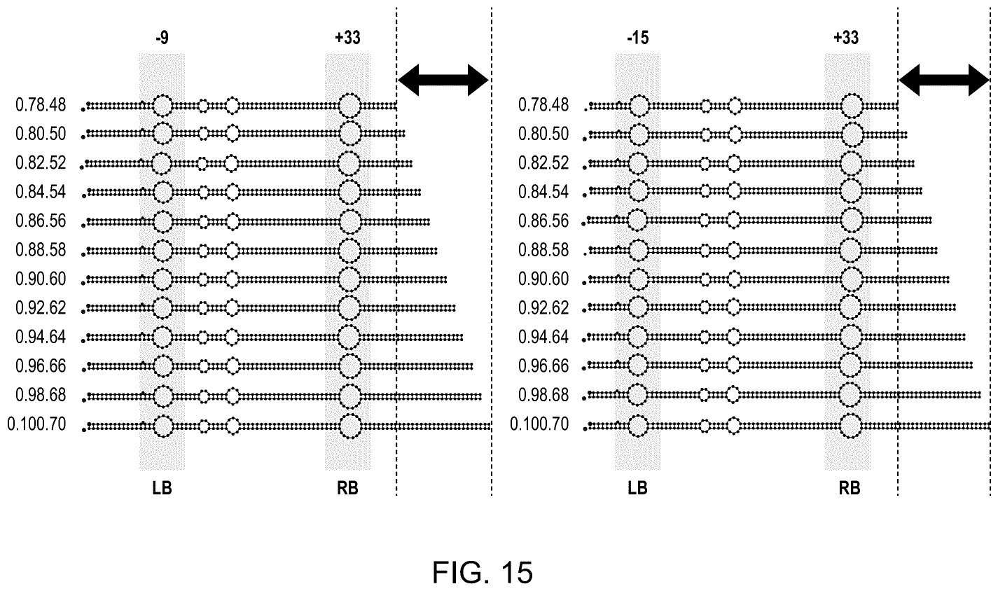

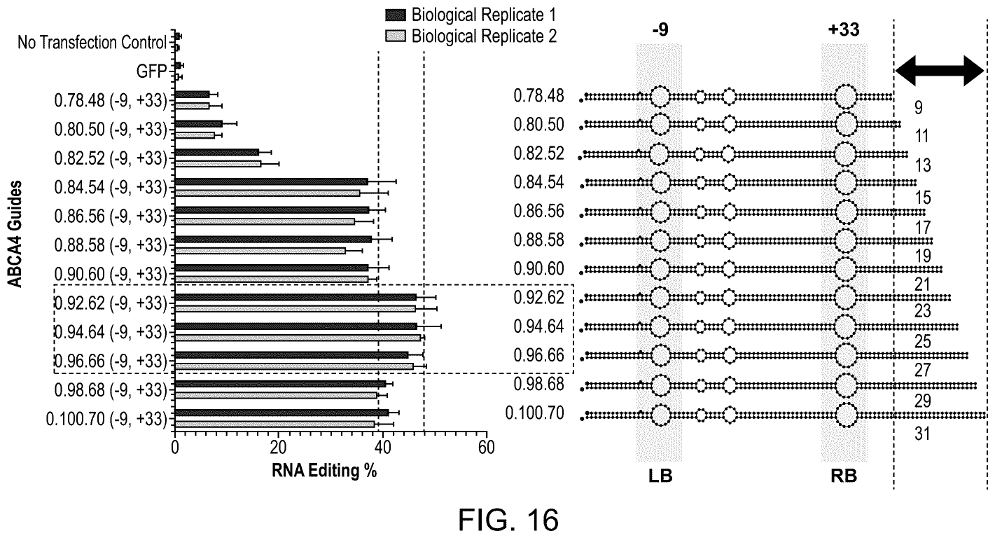

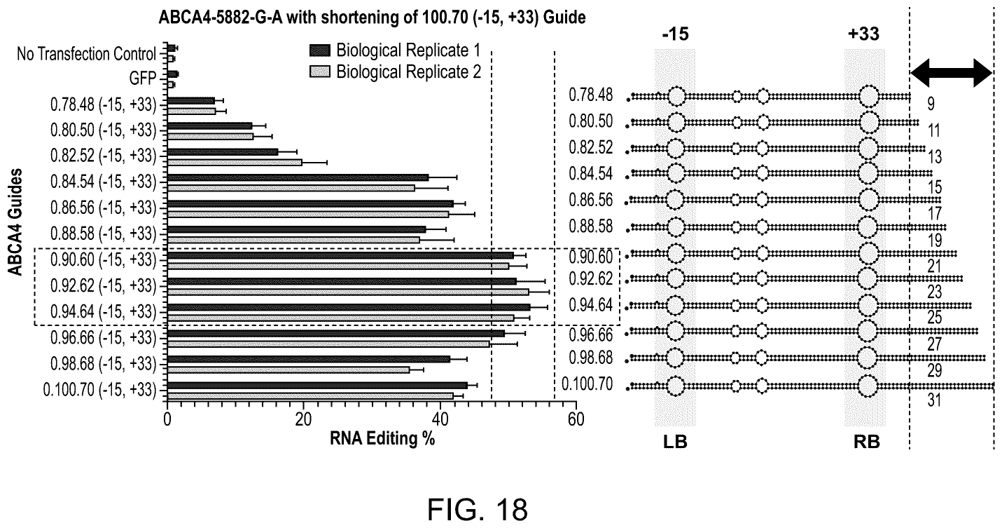

In some examples, the target RNA molecule can be encoded by, at least in part, an ABCA4 gene. In some examples, the ABCA4 gene comprises a mutation. In some examples, the mutation comprises a substitution of a G with an A at nucleotide position 5882 in an ABCA4 gene. In some examples, the mutation comprises a G with an A at nucleotide position 5714 in a ABCA4 gene. In some examples, the mutation comprises a substitution of a G with an A at nucleotide position 6320 in an ABCA4 gene. In some examples, the mutation causes or contributes to macular degeneration in a subject to which the engineered guide RNA is administered. In some examples, the macular degeneration can be Stargardt macular degeneration. In some examples the target RNA molecule comprises an adenosine with a 5′ G. In some examples, the adenosine with the 5′ G can be the base intended for chemical modification by the RNA editing entity. In some examples, the RNA editing entity can be an ADAR, and the ADAR chemically modifies the adenosine with the 5′ G after recruitment by the guide-target RNA scaffold. In some embodiments, an engineered guide RNA for targeting an ABCA4 mRNA can be any engineered guide depicted in FIG. 3, FIG. 4, FIG. 6, FIG. 7, FIG. 10, FIG. 11, FIG. 12, FIG. 13, FIG. 15, FIG. 16, or FIG. 18.

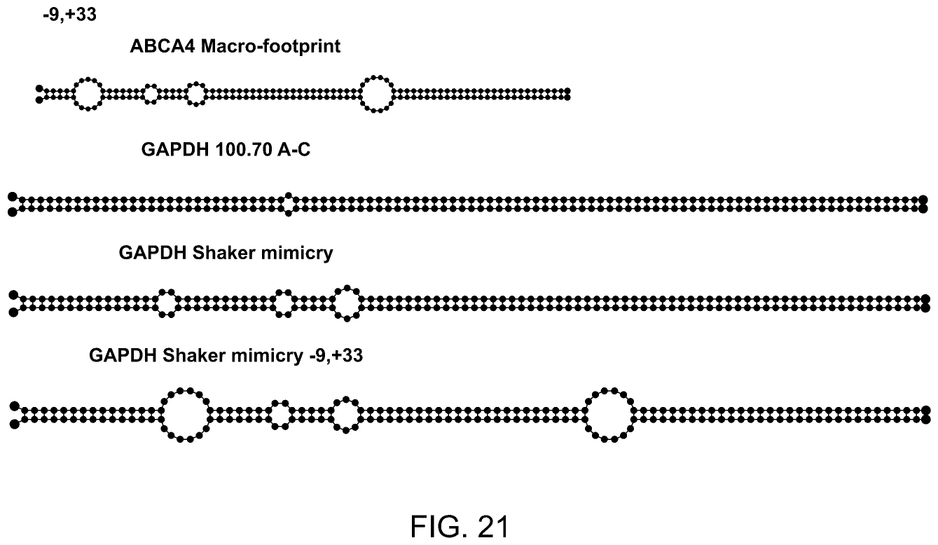

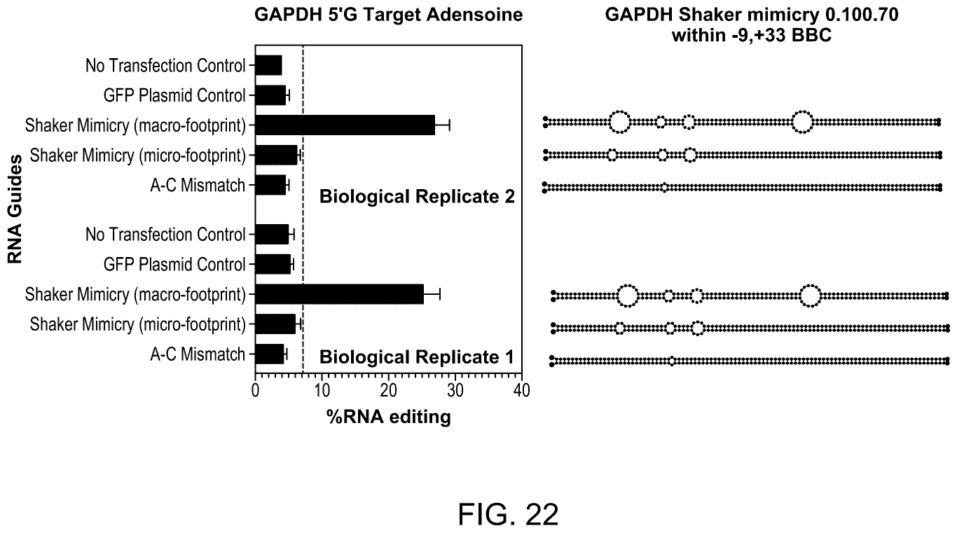

In some examples, the target RNA molecule can be encoded by, at least in part, a GAPDH gene. In some examples, the GAPDH gene comprises a mutation. In some examples, the mutation comprises a substitution of a G with an A at a nucleotide in a GAPDH gene. In some examples, the mutation causes or contributes to a neurological disease in a subject to which the engineered guide RNA is administered. In some examples, the neurological disease can be Alzheimer's disease. In some examples the target RNA molecule comprises an adenosine with a 5′ G. In some examples, the adenosine with the 5′ G can be the base intended for chemical modification by the RNA editing entity. In some examples, the RNA editing entity can be an ADAR, and the ADAR chemically modifies the adenosine with the 5′ G after recruitment by the double stranded substrate. In some embodiments, an engineered guide RNA for targeting a GAPDH mRNA can be any guide depicted in FIG. 21 or FIG. 22.

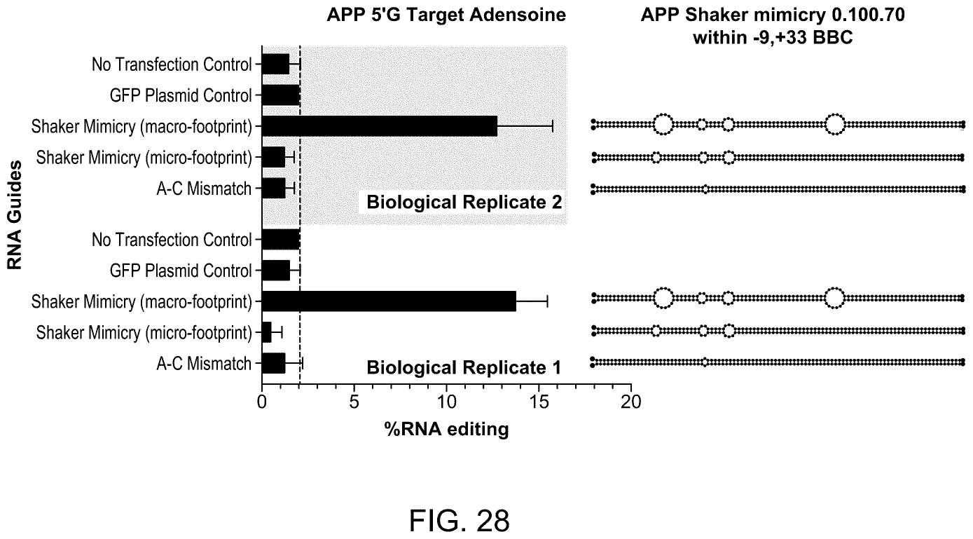

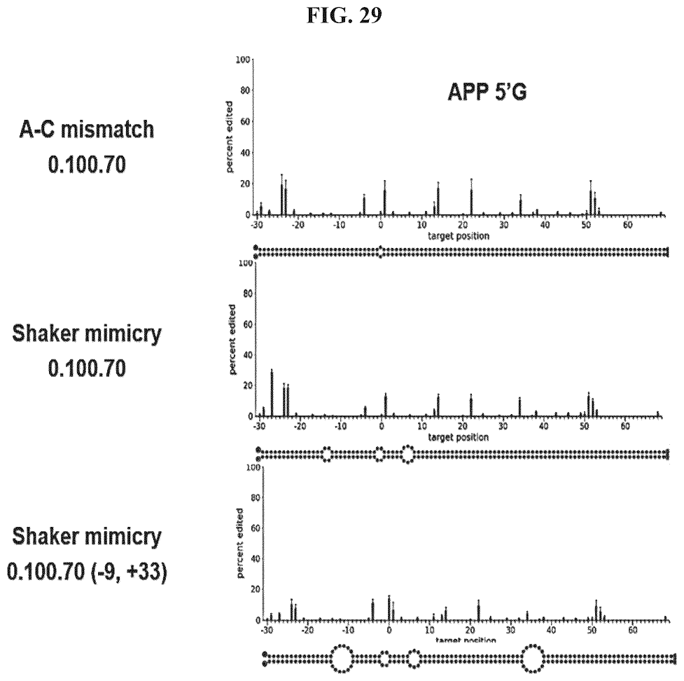

In some examples, the target RNA molecule can be encoded by, at least in part, an APP gene. In some examples, the APP gene comprises a mutation. In some examples, the mutation results in a mutation in an APP protein selected from the group consisting of: K670E, K670R, K670G, M671V, A673V, A673T, D672G, E682G, H684R, K687R, K687E, K687G, I712X, T714X, and any combination thereof. In some examples, the mutation causes or contributes to a neurological disease in a subject to which the engineered guide RNA is administered. In some examples, the neurological disease can be Alzheimer's disease, Parkinson's disease, corticobasal degeneration, dementia with Lewy bodies, Lewy body variant of Alzheimer's disease, Parkinson's disease with dementia, Pick's disease, progressive supranuclear palsy, dementia, fronto-temporal dementia with Parkinsonism linked to tau mutations on chromosome 17, or any combination thereof. In some examples the target RNA molecule comprises an adenosine with a 5′ G. In some examples, the adenosine with the 5′ G can be the base intended for chemical modification by the RNA editing entity. In some examples, the RNA editing entity can be an ADAR, and the ADAR chemically modifies the adenosine with the 5′ G after recruitment by the double stranded substrate. In some embodiments, an engineered guide RNA for targeting an APP mRNA can be any guide depicted in FIG. 28 or FIG. 29.

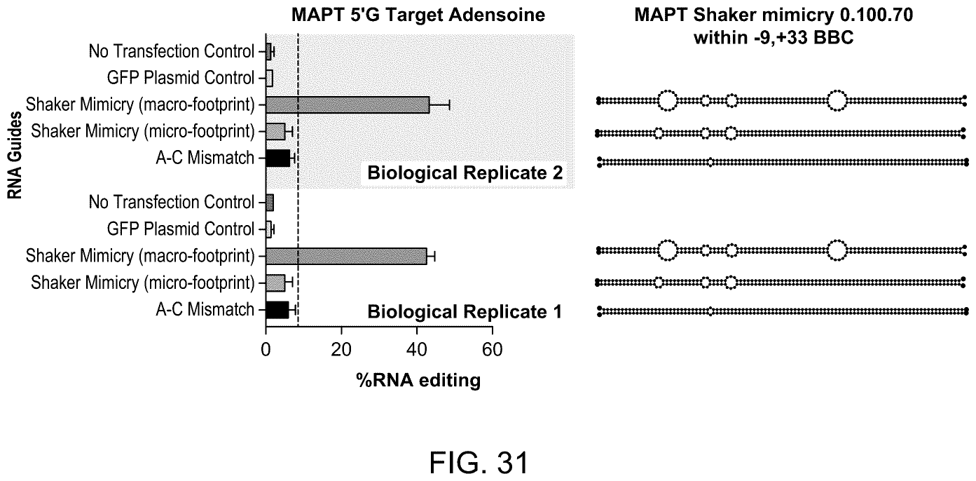

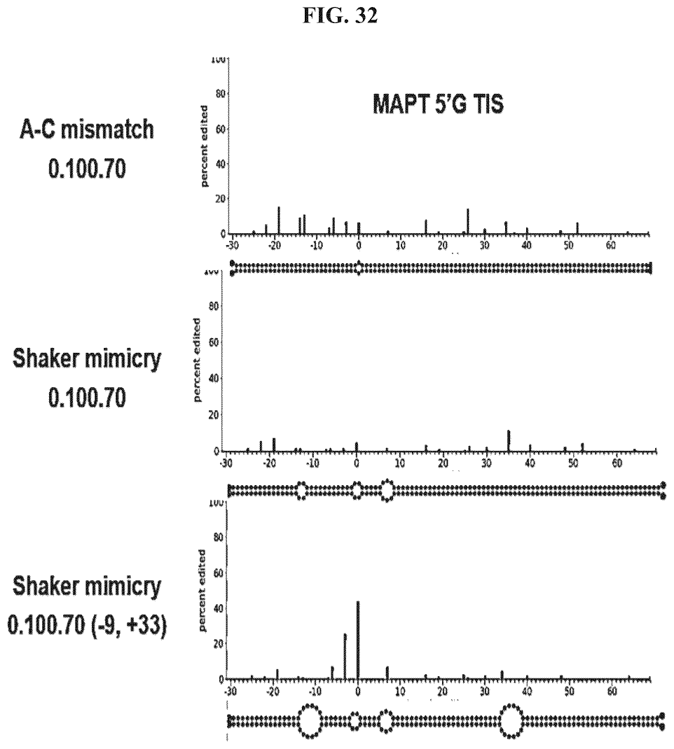

In some examples, the target RNA molecule can be encoded by, at least in part, a MAPT gene. In some examples, the MAPT gene comprises a mutation. In some examples, the mutation results in a mutation in a MAPT protein. In some examples, the mutation causes or contributes to a neurological disease in a subject to which the engineered guide RNA is administered. In some examples, the neurological disease can be Alzheimer's disease, Parkinson's disease, corticobasal degeneration, dementia with Lewy bodies, Lewy body variant of Alzheimer's disease, Parkinson's disease with dementia, Pick's disease, progressive supranuclear palsy, dementia, fronto-temporal dementia with Parkinsonism linked to tau mutations on chromosome 17, or any combination thereof. In some examples the target RNA molecule comprises an adenosine with a 5′ G. In some examples, the adenosine with the 5′ G can be the base intended for chemical modification by the RNA editing entity. In some examples, the RNA editing entity can be an ADAR, and the ADAR chemically modifies the adenosine with the 5′ G after recruitment by the double stranded substrate. In some embodiments, an engineered guide RNA for targeting a MAPT mRNA can be any guide depicted in FIG. 31 or FIG. 32.

In some cases, a targeting sequence comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that is bound by the targeting sequence can have a single base mismatch. In other cases, the targeting sequence of an engineered guide RNA comprises at least about 1, 2, 3, 4, 5, 6, 7, 8, 9, 10, 11, 12, 13, 14, 15, 16, 20, 30, 40 or up to about 50 base mismatches. In other cases, the targeting sequence of an engineered guide RNA comprises no more than about 1, 2, 3, 4, 5, 6, 7, 8, 9, 10, 11, 12, 13, 14, 15, 16, 20, 30, 40 or 50 base mismatches. In some examples, nucleotide mismatches are associated with structural features provided herein. In some examples, a targeting sequence comprises at least about 1, 2, 3, 4, 5, 6, 7, 8, 9, 10, 11, 12, 13, 14, or up to about 15 nucleotides that differ in complementarity from a wildtype RNA of a subject target RNA. In some examples, a targeting sequence comprises no more than about 1, 2, 3, 4, 5, 6, 7, 8, 9, 10, 11, 12, 13, 14, or 15 nucleotides that differ in complementarity from a wildtype RNA of a subject target RNA. In some cases, a targeting sequence comprises at least 50 nucleotides having complementarity to a target RNA. In some cases, a targeting sequence comprises from 50 to 150 nucleotides having complementarity to a target RNA. In some cases, a targeting sequence comprises from 50 to 200 nucleotides having complementarity to a target RNA. In some cases, a targeting sequence comprises from 50 to 250 nucleotides having complementarity to a target RNA. In some cases, a targeting sequence comprises from 50 to 300 nucleotides having complementarity to a target RNA. In some cases, a targeting sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 nucleotides having complementarity to a target RNA. In some cases, a targeting sequence comprises more than 50 nucleotides total and has at least 50 nucleotides having complementarity to a target RNA. In some cases, a targeting sequence comprises from 50 to 400 nucleotides total and has from 50 to 150 nucleotides having complementarity to a target RNA. In some cases, a targeting sequence comprises from 50 to 400 nucleotides total and has from 50 to 200 nucleotides having complementarity to a target RNA. In some cases, a targeting sequence comprises from 50 to 400 nucleotides total and has from 50 to 250 nucleotides having complementarity to a target RNA. In some cases, a targeting sequence comprises from 50 to 400 nucleotides total and has from 50 to 300 nucleotides having complementarity to a target RNA. In some cases, the at least 50 nucleotides having complementarity to a target RNA are separated by a structural feature described herein (e.g., one or more mismatches, one or more bulges, or one or more loops, one or more hairpins, or any combination thereof). In some cases, the 50 to 150 nucleotides having complementarity to a target RNA are separated by a structural feature described herein (e.g., one or more mismatches, one or more bulges, or one or more loops, one or more hairpins, or any combination thereof). In some cases, the 50 to 200 nucleotides having complementarity to a target RNA are separated by a structural feature described herein (e.g., one or more mismatches, one or more bulges, or one or more loops, one or more hairpins, or any combination thereof). In some cases, the 50 to 250 nucleotides having complementarity to a target RNA are separated by a structural feature described herein (e.g., one or more mismatches, one or more bulges, or one or more loops, one or more hairpins, or any combination thereof). In some cases, the 50 to 300 nucleotides having complementarity to a target RNA are separated by a structural feature described herein (e.g., one or more mismatches, one or more bulges, or one or more loops, one or more hairpins, or any combination thereof). For example, a targeting sequence can comprise a total of 54 nucleotides wherein, sequentially, 25 nucleotides are complementarity to a target RNA, 4 nucleotides form a bulge, and 25 nucleotides are complementarity to a target RNA. As another example, a targeting sequence comprises a total of 118 nucleotides wherein, sequentially, 25 nucleotides are complementarity to a target RNA, 4 nucleotides form a bulge, 25 nucleotides are complementarity to a target RNA, 14 nucleotides form an internal loop, and 50 nucleotides are complementary to a target RNA.

In some cases, an engineered guide RNA can comprise multiple targeting sequences. In some instances, one or more target sequence domains in the engineered guide RNA can bind to one or more regions of a target RNA. For example, a first targeting sequence can be configured to be at least partially complementary to a first region of a target RNA (e.g., a first exon of a pre-mRNA), while a second targeting sequence can be configured to be at least partially complementary to a second region of a target RNA (e.g., a second exon of a pre-mRNA). In some instances, multiple target sequences can be operatively linked to provide continuous hybridization of multiple regions of a target RNA. In some instances, multiple target sequences can provide non-continuous hybridization of multiple regions of a target RNA. A “non-continuous” overlap or hybridization refers to hybridization of a first region of a target RNA by a first targeting sequence, along with hybridization of a second region of a target RNA by a second targeting sequence, where the first region and the second region of the target RNA are discontinuous (e.g., where there is intervening sequence between the first and the second region of the target RNA). For example, a targeting sequence can be configured to bind to a portion of a first exon and can comprise an internal asymmetric loop (e.g., an oligo tether) that is configured to bind to a portion of a second exon, while the intervening sequence between the portion of exon 1 and the portion of exon 2 is not hybridized by either the targeting sequence or the oligo tether. Use of an engineered guide RNA as described herein configured for non-continuous hybridization can provide a number of benefits. For instance, such a guide can potentially target pre-mRNA during transcription (or shortly thereafter), which can then facilitate chemical modification using a deaminase (e.g., ADAR) co-transcriptionally and thus increase the overall efficiency of the chemical modification. Further, the use of oligo tethers to provide non-continuous hybridization while skipping intervening sequence can result in shorter, more specific guide RNA with fewer off-target editing.

In some instances, an engineered guide RNA configured for non-continuous hybridization to a target RNA (e.g., an engineered guide RNA comprising a targeting sequence with an oligo tether) can be configured to bind distinct regions or a target RNA separated by intervening sequence. In some instances, the intervening sequence can be at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 bases. In some instances, the targeting sequence and oligo tether can target distinct non-continuous regions of the same intron or exon. In some instances, the targeting sequence and oligo tether can target distinct non-continuous regions of adjacent exons or introns. In some instances, the targeting sequence and oligo tether can target distinct non-continuous regions of distal exons or introns.

### Circularized Guide RNA

In some instances, an engineered guide RNA can be circularized. A circularized engineered guide RNA can be produced from a precursor engineered polynucleotide. In some cases, a precursor engineered polynucleotide can be a precursor engineered linear polynucleotide. In some cases, a precursor engineered polynucleotide can be linear. For example, a precursor engineered polynucleotide can be a linear mRNA transcribed from a plasmid. In another example, a precursor engineered polynucleotide can be constructed to be a linear polynucleotide with domains such as a ribozyme domain and a ligation domain that allow for circularization in a cell. The linear polynucleotide with the ligation and ribozyme domains can be transfected into a cell where it can circularize via endogenous cellular enzymes. In some cases, a precursor engineered polynucleotide can be circular. In some cases, a precursor engineered polynucleotide can comprise DNA, RNA or both. In some cases, a precursor engineered polynucleotide can comprise a precursor engineered guide RNA. In some cases, a precursor engineered guide RNA can be used to produce an engineered guide RNA.

A circular or looped engineered guide polynucleotide, such as an engineered guide RNA can be formed directly or indirectly by forming a linkage (such as a covalent linkage) between more than one end of a RNA sequence, such as a 5′ end and a 3′ end. An RNA sequence can comprise an engineered guide RNA (such as a recruiting domain, targeting domain, or both). A linkage can be formed by employing an enzyme, such as a ligase. A suitable ligase (or synthetase) can include a ligase that forms a covalent bond. A covalent bond can include a carbon-oxygen bond, a carbon-sulfur bond, a carbon-nitrogen bond, a carbon-carbon bond, a phosphoric ester bond, or any combination thereof. A linkage can also be formed by employing a recombinase. An enzyme can be recruited to an RNA sequence to form a linkage. A circular or looped RNA can be formed by ligating more than one end of an RNA sequence using a linkage element. In some embodiments, a linkage can be formed by a ligation reaction. In some instances, a linkage can be formed by a homologous recombination reaction. A linkage element can employ click chemistry to form a circular or looped RNA. A linkage element can be an azide-based linkage. A circular or looped RNA can be formed by genetically encoding or chemically synthesizing the circular or looped RNA.

A circular or looped RNA can be formed by employing a self-cleaving entity, such as a ribozyme, tRNA, aptamer, catalytically active fragment of any of these, or any combination thereof. For example, a ribozyme, a tRNA, an aptamer, a catalytically active fragment of any of these, or any combination thereof can be added to a 3′ end, a 5′ end, or both of a precursor engineered RNA. In another example, a ribozyme, a tRNA, an aptamer, a catalytically active fragment of any of these, or any combination thereof can be added to a 3′ terminal end, a 5′ terminal end, or both of a precursor engineered RNA. A self-cleaving ribozyme can comprise, for example, an RNase P RNA a Hammerhead ribozyme (e.g. a Schistosoma mansoni ribozyme), a glmS ribozyme, an HDV-like ribozyme, an R2 element, a peptidyl transferase 23S rRNA, a GIR1 branching ribozyme, a leadzyme, a group II intron, a hairpin ribozyme, a VS ribozyme, a CPEB3 ribozyme, a CoTC ribozyme, or a group I intron. In some cases, the self-cleaving ribozyme can be a trans-acting ribozyme that joins one RNA end on which it is present to a separate RNA end. In some embodiments, an aptamer can be added to each end of the engineered guide RNA. A ligase can be contacted with the aptamers at each end of the engineered guide RNA to form a covalent linkage between the aptamers thereby forming a circular engineered guide RNA. In some cases, a self-cleaving element or an aptamer can be configured to facilitate self-circularization of an engineered polynucleotide or a pro-polynucleotide (e.g. from a precursor engineered polypeptide) after transcription in a cell. In some instances, circularization of a guide RNA can be shown by PCR. For example, primers can by developed that bind to the end of a guide RNA and are directed outward such that a product is only formed when guides are circularized.

In some cases, circularization can occur by back-slicing and ligation of an exon. For example, an RNA can be engineered from 5′ to 3′ to comprise a forward complementary sequence intron, an exon (which can comprise the guide sequence), followed by a reverse complementary sequence intron. Once transcribed, the complementary sequence introns can hybridize and form dsRNA. The internal exon containing the guide sequence can be removed by splicing and ligated by an endogenous ligase to form a circular guide. In one example, an engineered guide RNA can initiate circularization in a cell by autocatalytic reactions of encoded ribozymes. After cleavage by one or more ribozymes, the linear polynucleotide will undergo intracellular RNA ligation of the 5′ and the 3′ end of ligation sequences by an endogenous ligase to circularize the guide RNA.

A suitable self-cleaving molecule can include a ribozyme. For example, a ribozyme domain can create an autocatalytic RNA. A ribozyme can comprise an RNase P, an rRNA (such as a Peptidyl transferase 23S rRNA), Leadzyme, Group I intron ribozyme, Group II intron ribozyme, a GIR1 branching ribozyme, a glmS ribozyme, a hairpin ribozyme, a Hammerhead ribozyme, an HDV ribozyme, a Twister ribozyme, a Twister sister ribozyme, a VS ribozyme, a Pistol ribozyme, a Hatchet ribozyme, a viroid, or any combination thereof. A ribozyme can include a P3 twister U2A ribozyme. A ribozyme can comprise 5′ GCCATCAGTCGCCGGTCCCAAGCCCGGATAAAATGGGAGGGGGCGGGAAACCGCCT 3′ (SEQ ID NO: 3125). A ribozyme can comprise 5′ GCCAUCAGUCGCCGGUCCCAAGCCCGGAUAAAAUGGGAGGGGGCGGGAAACCGCC U 3′ (SEQ ID NO: 3126). A ribozyme can comprise at least about: 70%, 75%, 80%, 85%, 90%, 95%, or 100% sequence homology to 5′ GCCATCAGTCGCCGGTCCCAAGCCCGGATAAAATGGGAGGGGGCGGGAAACCGCCT 3′ (SEQ ID NO: 3125). A ribozyme can comprise at least about: 70%, 75%, 80%, 85%, 90%, 95%, or 100% sequence homology to 5′ GCCAUCAGUCGCCGGUCCCAAGCCCGGAUAAAAUGGGAGGGGGCGGGAAACCGCC U 3′ (SEQ ID NO: 3126). A ribozyme can include a P1 Twister Ribozyme. A ribozyme can include 5′ AACACTGCCAATGCCGGTCCCAAGCCCGGATAAAAGTGGAGGGTACAGTCCACGC 3′ (SEQ ID NO: 3127). A ribozyme can include 5′ AACACUGCCAAUGCCGGUCCCAAGCCCGGAUAAAAGUGGAGGGUACAGUCCACGC 3′ (SEQ ID NO: 3128). A ribozyme can comprise at least about: 70%, 75%, 80%, 85%, 90%, 95%, or 100% sequence homology to 5′ AACACTGCCAATGCCGGTCCCAAGCCCGGATAAAAGTGGAGGGTACAGTCCACGC 3′ (SEQ ID NO: 3127). A ribozyme can comprise at least about: 70%, 75%, 80%, 85%, 90%, 95%, or 100% sequence homology to 5′ AACACUGCCAAUGCCGGUCCCAAGCCCGGAUAAAAGUGGAGGGUACAGUCCACGC 3′ (SEQ ID NO: 3128).

A ligation domain can facilitate a linkage, covalent or non-covalent, of a first nucleotide to a second nucleotide. In some embodiments, a ligation domain can recruit a ligating entity to facilitate a ligation reaction. In some cases, a ligation domain can recruit a recombining entity to facilitate a homologous recombination. In some instances, a first ligation domain can facilitate a linkage, covalent or non-covalent, to a second ligation domain. In some embodiments, a first ligation domain can facilitate the complementary pairing of a second ligation domain. In some cases, a ligation domain can comprise 5′ AACCATGCCGACTGATGGCAG 3′ (SEQ ID NO: 3129). In some embodiments, a ligation domain can comprise 5′ GATGTCAGGTGCGGCTGACTACCGTC 3′ (SEQ ID NO: 3130). In some cases, a ligation domain can comprise 5′ AACCAUGCCGACUGAUGGCAG 3′ (SEQ ID NO: 3131). In some cases, a ligation domain can comprise 5′ GAUGUCAGGUGCGGCUGACUACCGUC 3′ (SEQ ID NO: 3132). In some cases, a ligation domain can comprise at least about: 70%, 75%, 80%, 85%, 90%, 95%, or 100% sequence homology to 5′AACCATGCCGACTGATGGCAG 3′ (SEQ ID NO: 3129). In some cases, a ligation domain can comprise at least about: 70%, 75%, 80%, 85%, 90%, 95%, or 100% sequence homology to 5′ GATGTCAGGTGCGGCTGACTACCGTC 3′ (SEQ ID NO: 3130). In some cases, a ligation domain can comprise at least about: 70%, 75%, 80%, 85%, 90%, 95%, or 100% sequence homology to 5′ AACCAUGCCGACUGAUGGCAG 3′ (SEQ ID NO: 3131). In some cases, a ligation domain can comprise at least about: 70%, 75%, 80%, 85%, 90%, 95%, or 100% sequence homology to 5′ GAUGUCAGGUGCGGCUGACUACCGUC 3′ (SEQ ID NO: 3132).

### Bolt-on Recruiting Domains

In some embodiments, an engineered guide RNA having a barbell macro-footprint sequence can further comprise an RNA editing entity recruiting domain configured to be formed and present in the absence of hybridization to a target RNA.

A “recruiting domain” can be referred to herein interchangeably as a “recruiting sequence” or a “recruiting region.” In some examples, an engineered guide RNA can be configured to facilitate editing of a base of a nucleotide of a polynucleotide of a region of a target RNA, modulation expression of a polypeptide encoded by the target RNA, or both. In some cases, an engineered guide RNA can be configured to facilitate an editing of a base of a nucleotide or polynucleotide of a region of an RNA by an RNA editing entity. In order to facilitate editing, an engineered guide RNA of the disclosure can be configured to recruit an RNA editing entity. Some embodiments provide for an RNA editing entity comprising an ADAR protein, where the ADAR protein can be selected from the group consisting of an ADAR1 (e.g., human or mouse), an ADAR2 (e.g., human or mouse), and any combination thereof. Various RNA editing entity recruiting domains can be utilized. In some examples, a recruiting domain comprises: Glutamate ionotropic receptor AMPA type subunit 2 (GluR2) or Alu. In some embodiments of the disclosure, the RNA editing entity can have an ADAR protein. An ADAR protein can be selected from the group consisting of: an ADAR1, an ADAR2, and a combination of ADAR1 and ADAR2. Other embodiments can be directed to an RNA editing entity selected from the group consisting of: a human ADAR1, a mouse ADAR1, a human ADAR2, a mouse ADAR2, and any combination thereof.

In some examples, more than one recruiting domain can be included in an engineered guide RNA of the disclosure. In examples where a recruiting domain can be present, the recruiting domain can be utilized to position the RNA editing entity to effectively react with a target RNA after the targeting sequence, for example an antisense sequence, hybridizes to a target RNA. In some cases, a recruiting domain can allow for transient binding of the RNA editing entity to the engineered guide RNA. In some examples, the recruiting domain allows for permanent binding of the RNA editing entity to the engineered guide RNA. A recruiting domain can be of any length. In some cases, a recruiting domain can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting domain can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting domain can be about 45 nucleotides in length. In some cases, at least a portion of a recruiting domain comprises at least 1 to about 75 nucleotides. In some cases, at least a portion of a recruiting domain comprises about 45 nucleotides to about 60 nucleotides.

In some embodiments, a recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 70%, 80%, 85%, 90%, 95%, 98%, 99%, or 100% identity, length, or both to: GUGGAAUAGUAUAACAAUAUGCUAAAUGUUGUUAUAGUAUCCCAC (SEQ ID NO:1). In some cases, a recruiting domain can comprise at least about 80% sequence homology to at least about 10, 15, 20, 25, or 30 nucleotides of SEQ ID NO:1. In some examples, a recruiting domain can comprise at least about 90%, 95%, 96%, 97%, 98%, or 99% sequence homology, length, or both to SEQ ID NO:1.

Any number of recruiting domains can be found in an engineered RNA of the present disclosure. In some examples, at least about 1, 2, 3, 4, 5, 6, 7, 8, 9, or up to about 10 recruiting domains can be included in an engineered RNA. Recruiting domains can be located at any position of an engineered guide RNA. In some cases, a recruiting domain can be on an N-terminus, middle, or C-terminus of a polynucleotide. A recruiting domain can be upstream or downstream of a targeting sequence. In some cases, a recruiting domain flanks a targeting sequence of a guide. A recruiting sequence can comprise all ribonucleotides or deoxyribonucleotides, although a recruiting domain comprising both ribo- and deoxy-ribonucleotides can in some cases not be excluded.

### Multiplexed Therapy

In some cases, the present disclosure encompasses multiplexed therapy, including multiplexed editing of multiple target RNAs, editing of multiple target sites within a target RNA (e.g., for missense mutation correction and functional protein restoration), editing of RNA and knockdown, or any combination thereof. In some cases, use of vectors that contains multiple targeting guide RNAs can allow for simultaneous targeting of multiple gene targets or multiple sites within the same gene target. Any combination of gene targets disclosed herein can be targeted using a multiplexed approach to treat a particular genetic disease. As the compositions can be applied to gene expression knockdown, they could also include a combination of start-site editing to reduce expression, steric hinderance because the guide could block ribosomal activity, increased degradation of the targeted mRNA, or any combination thereof. The compositions and methods disclosed herein, thus, may suppress expression in an ADAR-dependent and ADAR-independent manner.

In some embodiments, a multiplex approach can be used to target Abeta, Tau, or SNCA target RNAs. Both Abeta and Tau or SNCA are implicated in Alzheimer's disease initiation/progression. The compositions and methods disclosed herein can target both and, thus, may involve a multiplexed targeting approach. A multiplexed targeting approach can target 2, 3, 4, 5, 6, or more proteinopathies by independent mechanisms of action. For example, mRNA base editing using the engineered guide RNAs of the present disclosure can edit one or more cleavage sites of an APP protein preventing or substantially reducing Abeta fragment formation and mRNA base editing using the engineered guide RNAs of the present disclosure can knockdown Tau protein formation. In some cases, mRNA base editing using the guide RNAs of the present disclosure can edit one or more cleavage sites of an APP protein preventing or substantially reducing Abeta fragment formation and an additional therapeutic agent (e.g., an additional RNA polynucleotide, such as a siRNA, a shRNA, a miRNA, a piRNA, or an antisense oligonucleotide), which can knockdown Tau protein formation.

In some embodiments, a vector of the present disclosure may be a multiplex vector that contain multiple engineered guide polynucleotides targeting multiple target RNAs. In other cases, different engineered guide polynucleotide targeting different target RNAs can be maintained on different vectors. Vectors encoding for compositions that can (a) facilitate an edit to a target RNA (e.g., a cleavage site of a protein target, such as is the case in targeting the beta secretase cleavage site in APP, to a TIS or UTR region for protein knockdown, to a missense mutation to restore the wild-type sequence and thus restore protein expression, or other targeting strategies described herein), (c) reduce or regulate the activity of a protein target produced, (d) correct a mutation and thereby restore functional protein expression, or (e) any combination thereof. A vector or a multiplex vector or vectors can be formulated in unit dose form. A multiplex vector can be configured to modulate more than one protein target implicated in a neurodegenerative disease. A vector can reduce an amount of the protein target by (i) performing an edit to a sequence that encodes for the protein, (ii) performing an edit to a sequence that does not encode for the protein, (iii) sterically hindering a promoter region associated with the protein target, or (iv) any combination thereof.

In some cases, polynucleotide base editing can be used in conjunction with an additional method of knocking down gene expression of either the same gene targeted by the polynucleotide base editing or an additional gene implicated in a disease, such as a neurodegenerative disease. For example, mRNA base editing (e.g. using the engineered guide RNAs disclosed herein) can be used in conjunction with an RNA polynucleotide that associates with an mRNA sequence to minimize expression of a targeted gene. Examples of such RNA polynucleotides capable of minimizing expression of a targeted gene include small interfering RNA (siRNA), short hairpin RNA (shRNA), microRNA (miRNA), piwi-interacting RNA (piRNA), or an anti-sense oligonucleotide (ASO). In some cases, the ASO comprises a variant oligonucleotide structure that stabilizes the oligonucleotide and/or minimizes nuclease activity on the nucleotide. Examples of such variants oligonucleotides include morpholino oligomers. Thus, the present disclosure provides for compositions of engineered guides RNAs in combination with one or more additional therapeutic agents selected from small interfering RNA (siRNA), short hairpin RNA (shRNA), microRNA (miRNA), piwi-interacting RNA (piRNA), and an antisense oligonucleotide (ASO).

An mRNA base editing approach using an engineered polynucleotide, such as guide RNA, of the present disclosure can be combined with an antibody-based approach (such as anti-Abeta antibodies). Disadvantages to employing an antibody-based approach alone can include low/inefficient transfer across the blood-brain-barrier and development of ARIA (neuroinflammation) in patients treated with these therapies constrain the therapeutic dose. It is likely that more than one Abeta species (including soluble Abeta) contribute to disease progression. Thus, multiple different antibodies to the different species can be needed. Antibodies that may be combined with the engineered guide RNAs disclosed herein for combination treatment of a subject in need thereof can include bapineuzumab, solanezumab, gantenerumab, crenezumab, ponezumab, aducanumab, BAN2401, or any combination thereof.

An mRNA base editing approach using the engineered polynucleotides of the present disclosure can be combined with a secretase inhibitor approach (such as a beta secretase and γ-secretase inhibitors). Both enzymes appear to have proteolytic activity necessary for maintenance of synaptic function/neuronal health and thus severely or completely reducing function can led to poor long-term outcomes. Utilizing a combined approach of an mRNA base editing and secretase inhibitor approach can permit reducing dosing of the secretase inhibitor as compared to a solitary approach of delivering the secretase inhibitor alone. Inhibitors that may be combined with the engineered guide RNAs disclosed herein for combination treatment of a subject in need thereof can include verubecestat, atabecestat, lanabecestat, elenbecestat, umibecestat, avagacestat, semagacestat, or any combination thereof.

### Pharmaceutical Compositions

An engineered guide RNA described herein (e.g., a guide RNA comprising abarbell macro-footprint sequence and at least some elements of a micro-footprint sequence) or a polynucleotide encoding the same can be formulated with a pharmaceutically acceptable carrier for administration to a subject (e.g., a human or a non-human animal).

The compositions described herein (e.g., compositions comprising an engineered guide RNA or polynucleotide encoding an engineered guide RNA of the disclosure) can be formulated with a pharmaceutically acceptable carrier, diluent, or excipient for administration to a subject (e.g., a human or a non-human animal). A pharmaceutically acceptable carrier can include, but is not limited to, phosphate buffered saline solution, water, emulsions (e.g., an oil/water emulsion or a water/oil emulsions), glycerol, liquid polyethylene glycols, aprotic solvents such (e.g., dimethylsulfoxide, N-methylpyrrolidone, or mixtures thereof), and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. Additional examples of carriers, diluents, excipients, stabilizers, and adjuvants consistent with the compositions of the present disclosure can be found in, for example, Remington's Pharmaceutical Sciences, 21st Ed., Mack Publ. Co., Easton, Pa. (2005), incorporated herein by reference in its entirety.

### Chemical Modification

An engineered guide RNA as described herein for use in treating a disease or condition in a subject comprises at least one chemical modification. In some embodiments, the engineered guide RNA comprises at least one, two, three, four, five, six, seven, eight, nine, ten, 11, 12, 13, 14, 15, 16, 17, 18, 19, 20, 30, 50, 100, or more chemical modifications. In some cases, the engineered guide RNAs disclosed herein with barbell macro-footprints can be manufactured, chemically modified, and delivered directly to a subject in need thereof as RNA (without a vector, such as an AAV).

Exemplary chemical modifications comprise any one of: 5′ adenylate, 5′ guanosine-triphosphate cap, 5′ N7-Methylguanosine-triphosphate cap, 5′ triphosphate cap, 3′ phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3phosphorothioate or any combinations thereof.

A chemical modification can be made at any location of the engineered guide RNA. In some cases, a modification may be located in a 5′ or 3′ end. In some cases, a polynucleotide comprises a modification at a base selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150. More than one modification can be made to the engineered guide RNA. In some cases, a modification can be permanent. In other cases, a modification can be transient. In some cases, multiple modifications may be made to the engineered guide RNA. the engineered guide RNA modification can alter physio-chemical properties of a nucleotide, such as their conformation, polarity, hydrophobicity, chemical reactivity, base-pairing interactions, or any combination thereof.

A chemical modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases may be of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

In some embodiments, chemical modification can occur at 3′OH, group, 5′OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some embodiments, chemically modified backbone comprises a backbone other than a phosphodiester backbone. In some embodiments, a modified sugar comprises a sugar other than deoxyribose (in modified DNA) or other than ribose (modified RNA). In some embodiments, a modified base comprises a base other than adenine, guanine, cytosine, thymine or uracil. In some embodiments, the engineered guide RNA comprises at least one chemically modified base. In some instances, the engineered guide RNA comprises 2, 3, 4, 5, 6, 7, 8, 9, 10, 15, 20, or more modified bases. In some cases, chemical modifications to the base moiety include natural and synthetic modifications of adenine, guanine, cytosine, thymine, or uracil, and purine or pyrimidine bases.

In some embodiments, the at least one chemical modification of the engineered guide RNA comprises a modification of any one of or any combination of: modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Chemical modifications to the engineered guide RNA include any modification contained herein, while some exemplary modifications are recited in Table 2.

TABLE 2
Exemplary Chemical Modification

| Modification of | |
|---|---|
| engineered guide RNA | Examples |
| Modification of one or both | sulfur (S), selenium (Se), BR3 (wherein R can be, e.g., hydrogen, |
| of the non-linking | alkyl, or aryl), C (e.g., an alkyl group, an aryl group, and the like), |
| phosphate oxygens in the | H, NR2, wherein R can be, e.g., hydrogen, alkyl, or aryl, or |
| phosphodiester backbone | wherein R can be, e.g., alkyl or aryl |
| linkage | |
| Modification of one or more | sulfur (S), selenium (Se), BR3 (wherein R can be, e.g., hydrogen, |
| of the linking phosphate | alkyl, or aryl), C (e.g., an alkyl group, an aryl group, and the like), |
| oxygens in the | H, NR2, wherein R can be, e.g., hydrogen, alkyl, or aryl, or |
| phosphodiester backbone | wherein R can be, e.g., alkyl or aryl |
| linkage | |
| Replacement of the | methyl phosphonate, hydroxylamino, siloxane, carbonate, |
| phosphate moiety with | carboxymethyl, carbamate, amide, thioether, ethylene oxide linker, |
| “dephospho” linkers | sulfonate, sulfonamide, thioformacetal, formacetal, oxime, |
| | methyleneimino, methylenemethylimino, methylenehydrazo, |
| | methylenedimethylhydrazo, or methyleneoxymethylimino |
| Modification or | Nucleic acid analog (examples of nucleotide analogs can be found |
| replacement of a naturally | in PCT/US2015/025175, PCT/US2014/050423, |
| occurring nucleobase | PCT/US2016/067353, PCT/US2018/041503, PCT/US18/041509, |
| | PCT/US2004/011786, or PCT/US2004/011833, all of which are |
| | expressly incorporated by reference in their entireties |
| Modification of the ribose- | phosphorothioate, phosphonothioacetate, phosphoroselenates, |
| phosphate backbone | boranophosphates, borano phosphate esters, hydrogen |
| | phosphonates, phosphonocarboxylate, phosphoroamidates, alkyl or |
| | aryl phosphonates, phosphonoacetate, or phosphotriesters |
| Modification of 5′ end of | 5′ cap or modification of 5′ cap —OH |
| polynucleotide | |
| Modification of 3′ end of | 3′ tail or modification of 3′ end —OH |
| polynucleotide | |
| Modification of the | phosphorothioate, phosphonothioacetate, phosphoroselenates, |
| deoxyribose phosphate | borano phosphates, borano phosphate esters, hydrogen |
| backbone | phosphonates, phosphoroamidates, alkyl or aryl phosphonates, or |
| | phosphotriesters |
| Substitution of the | methyl phosphonate, hydroxylamino, siloxane, carbonate, |
| phosphate group | carboxymethyl, carbamate, amide, thioether, ethylene oxide linker, |
| | sulfonate, sulfonamide, thioformacetal, formacetal, oxime, |
| | methyleneimino, methylenemethylimino, methylenehydrazo, |
| | methylenedimethylhydrazo, or methyleneoxymethylimino. |
| Modification of the | morpholino, cyclobutyl, pyrrolidine, or peptide nucleic acid (PNA) |
| ribophosphate backbone | nucleoside surrogates |
| Modifications to the sugar | Locked nucleic acid (LNA), unlocked nucleic acid (UNA), or |
| of a nucleotide | bridged nucleic acid (BNA) |
| Modification of a | 2′-O-methyl, 2′-O-methoxy-ethyl (2′-MOE), 2′-fluoro, 2′- |
| constituent of the ribose | aminoethyl, 2′-deoxy-2′-fuloarabinou-cleic acid, 2′-deoxy, 2′-O- |
| sugar | methyl, 3′-phosphorothioate, 3′-phosphonoacetate (PACE), or 3′- |
| | phosphonothioacetate (thioPACE) |
| Modifications to the base of | Modification of A, T, C, G, or U |
| a nucleotide | |
| Stereopure of nucleotide | S conformation of phosphorothioate or R conformation of |
| | phosphorothioate |

### Modification of Phosphate Backbone

In some embodiments, the chemical modification comprises modification of one or both of the non-linking phosphate oxygens in the phosphodiester backbone linkage or modification of one or more of the linking phosphate oxygens in the phosphodiester backbone linkage. As used herein, “alkyl” may be meant to refer to a saturated hydrocarbon group which may be straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” may refer to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some embodiments, aryl groups have from 6 to about 20 carbon atoms. As used herein, “alkenyl” may refer to an aliphatic group containing at least one double bond. As used herein, “alkynyl” may refer to a straight or branched hydrocarbon chain containing 2-12 carbon atoms and characterized in having one or more triple bonds. Examples of alkynyl groups can include ethynyl, propargyl, or 3-hexynyl. “Arylalkyl” or “aralkyl” may refer to an alkyl moiety in which an alkyl hydrogen atom may be replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” may refer to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” may refer to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” may refer to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some embodiments, the phosphate group of a chemically modified nucleotide can be modified by replacing one or more of the oxygens with a different substituent. In some embodiments, the chemically modified nucleotide can include replacement of an unmodified phosphate moiety with a modified phosphate as described herein. In some embodiments, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way may be a stereogenic center. The stereogenic phosphorous atom can possess either the “R” configuration (herein Rp) or the “S” configuration (herein Sp). In some cases, the engineered guide RNA can comprise stereopure nucleotides comprising S conformation of phosphorothioate or R conformation of phosphorothioate. In some embodiments, the chiral phosphate product may be present in a diastereomeric excess of 50%, 60%, 70%, 80%, 90%, or more. In some embodiments, the chiral phosphate product may be present in a diastereomeric excess of 95%. In some embodiments, the chiral phosphate product may be present in a diastereomeric excess of 96%. In some embodiments, the chiral phosphate product may be present in a diastereomeric excess of 97%. In some embodiments, the chiral phosphate product may be present in a diastereomeric excess of 98%. In some embodiments, the chiral phosphate product may be present in a diastereomeric excess of 99%. In some embodiments, both non-bridging oxygens of phosphorodithioates can be replaced by sulfur. The phosphorus center in the phosphorodithioates can be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl). In some embodiments, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) may be attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It may be also understood in a nucleotide substitute that both the sugar and the phosphate moieties of the nucleotide can be replaced, by for example an amide type linkage (aminoethylglycine) (PNA). It may be also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some embodiments, the chemical modification described herein comprises modification of a phosphate backbone. In some embodiments, the engineered guide RNA described herein comprises at least one chemically modified phosphate backbone. Exemplary chemically modification of the phosphate group or backbone can include replacing one or more of the oxygens with a different substituent. Furthermore, the modified nucleotide present in the engineered guide RNA can include the replacement of an unmodified phosphate moiety with a modified phosphate as described herein. In some embodiments, the modification of the phosphate backbone can include alterations resulting in either an uncharged linker or a charged linker with unsymmetrical charge distribution. Exemplary modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or OR (wherein R can be, e.g., alkyl or aryl). The phosphorous atom in an unmodified phosphate group may be achiral. However, replacement of one of the non-bridging oxygens with one of the above atoms or groups of atoms can render the phosphorous atom chiral; that may be to say that a phosphorous atom in a phosphate group modified in this way may be a stereogenic center. The stereogenic phosphorous atom can possess either the “R” configuration (herein Rp) or the “S” configuration (herein Sp). In such case, the chemically modified engineered guide RNA can be stereopure (e.g. S or R confirmation). In some cases, the chemically modified engineered guide RNA comprises stereopure phosphate modification. For example, the chemically modified engineered guide RNA can comprise S conformation of phosphorothioate or R conformation of phosphorothioate.

Phosphorodithioates have both non-bridging oxygens replaced by sulfur. The phosphorus center in the phosphorodithioates may be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

he phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of Phosphate Moiety

In some embodiments, at least one phosphate group of the engineered guide RNA can be chemically modified. In some embodiments, the phosphate group can be replaced by non-phosphorus containing connectors. In some embodiments, the phosphate moiety can be replaced by dephospho linker. In some embodiments, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some embodiments, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g. 3′-amino phosphoramidate and aminoalkylphosphoramidates), thionophosphoramidates, thionoalkylphosphonates, thionoalkylphosphotriesters, and boranophosphates. The phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

### Substitution of Phosphate Group

In some embodiments, the chemical modification described herein comprises modification by replacement of a phosphate group. In some embodiments, the engineered guide RNA described herein comprises at least one chemically modification comprising a phosphate group substitution or replacement. Exemplary phosphate group replacement can include non-phosphorus containing connectors. In some embodiments, the phosphate group substitution or replacement can include replacing charged phosphate group can by a neutral moiety. Exemplary moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### Modification of the Ribophosphate Backbone

In some embodiments, the chemical modification described herein comprises modifying ribophosphate backbone of the engineered guide RNA. In some embodiments, the engineered guide RNA described herein comprises at least one chemically modified ribophosphate backbone. Exemplary chemically modified ribophosphate backbone can include scaffolds that can mimic nucleic acids can also be constructed wherein the phosphate linker and ribose sugar may be replaced by nuclease resistant nucleoside or nucleotide surrogates. In some embodiments, the nucleobases can be tethered by a surrogate backbone. Examples can include morpholino, cyclobutyl, pyrrolidine and peptide nucleic acid (PNA) nucleoside surrogates.

### Modification of Sugar

In some embodiments, the chemical modification described herein comprises modifying of sugar. In some embodiments, the engineered guide RNA described herein comprises at least one chemically modified sugar. Exemplary chemically modified sugar can include 2′ hydroxyl group (OH) modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH2CH₂OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include (LNA, in which the 2′ hydroxyl can be connected, e.g., by a Ci-₆ alkylene or Cj-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH₂CH₂OCH₃, e.g., a PEG derivative). In some cases, the deoxy modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH2CH₂-amino (wherein amino can be, e.g., as described herein),NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the F position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that may be in the L form, e.g. L-nucleosides. In some aspects, the engineered guide RNA described herein includes the sugar group ribose, which may be a 5-membered ring having an oxygen. Exemplary modified nucleosides and modified nucleotides can include replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose may be replaced by glycol units attached to phosphodiester bonds), threose nucleic acid. In some embodiments, the modifications to the sugar of the engineered guide RNA comprises modifying the engineered guide RNA to include locked nucleic acid (LNA), unlocked nucleic acid (UNA), or bridged nucleic acid (BNA).

### Modification of a Constituent of the Ribose Sugar

In some embodiments, the engineered guide RNA described herein comprises at least one chemical modification of a constituent of the ribose sugar. In some embodiments, the chemical modification of the constituent of the ribose sugar can include 2′-O-methyl, 2′-O-methoxy-ethyl (2′-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE). In some embodiments, the chemical modification of the constituent of the ribose sugar comprises unnatural nucleic acid. In some instances, the unnatural nucleic acids include modifications at the 5′-position and the 2′-position of the sugar ring, such as 5′-CH₂-substituted 2′-O-protected nucleosides. In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position may be unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂) (R=H, C₁-C₁₂ alkyl or a protecting group); and combinations thereof.

In some instances, the engineered guide RNA described herein comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl, 2′ sugar modifications also include but are not limited to—O[(CH₂)ₙO]ₘ CH₃,—O(CH₂)ₙOCH₃,—O(CH₂)ₙNH₂,—O(CH₂)ₙCH₃,—O(CH₂)ₙONH₂, and —O(CH₂)ₙON[(CH₂)n CH₃)]₂, where n and m may be from 1 to about 10. Other chemical modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof; 4′-C(CH₃)(CH₃)—O-2′and analogs thereof.

### Modifications on the Base of Nucleotide

In some embodiments, the chemical modification described herein comprises modification of the base of nucleotide (e.g. the nucleobase). Exemplary nucleobases can include adenine (A), thymine (T), guanine (G), cytosine (C), and uracil (U). These nucleobases can be modified or replaced to in the engineered guide RNA described herein. The nucleobase of the nucleotide can be independently selected from a purine, a pyrimidine, a purine or pyrimidine analog. In some embodiments, the nucleobase can be naturally-occurring or synthetic derivatives of a base.

In some embodiments, the chemical modification described herein comprises modifying an uracil. In some embodiments, the engineered guide RNA described herein comprises at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 1 methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N₁-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethy])-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

In some embodiments, the chemical modification described herein comprises modifying a cytosine. In some embodiments, the engineered guide RNA described herein comprises at least one chemically modified cytosine. Exemplary chemically modified cytosine can include 5-aza-cytidine, 6-aza-cytidine, pseudoisocytidine, 3-methyl-cytidine, N₄-acetyl-cytidine, 5-formyl-cytidine, N₄-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N₄-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some embodiments, the chemical modification described herein comprises modifying a adenine. In some embodiments, the engineered guide RNA described herein comprises at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′-O-dimethyl-adenosine, N6-Methyl-2′-deoxyadenosine, N6, N6, 2′-O-trimethyl-adenosine, 1,2′-O-dimethyl-adenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the chemical modification described herein comprises modifying a guanine. In some embodiments, the engineered guide RNA described herein comprises at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undemriodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′-O-dimethyl-inosine, 6-O-phenyl-2′-deoxyinosine, 2′-O-ribosylguanosine, 1-thio-guanosine, 6-O-methyguanosine, O⁶-Methyl-2′-deoxyguanosine, 2′-F-ara-guanosine, and 2′-F-guanosine.

In some cases, the chemical modification of the engineered guide RNA can include introducing or substituting a nucleic acid analog or an unnatural nucleic acid into the engineered guide RNA. In some embodiments, nucleic acid analog can be any one of the chemically modified nucleic acid described herein. Exemplary nucleic acid analog can be found in PCT/US2021/034272, PCT/US2015/025175, PCT/US2014/050423, PCT/US2016/067353, PCT/US2018/041503, PCT/US18/041509, PCT/US2004/011786, or PCT/US2004/011833, all of which are expressly incorporated by reference in their entireties. The chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Exemplary chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2, —O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-ami nohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. In some embodiments, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 6-methylguanosine-5′-triphosphate, pseudouridine-5′-triphosphate, puromycin-5′-triphosphate, or xanthosine-5′-triphosphate. In some embodiments, the chemically modified nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-tauri nomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-th io-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the chemically modified nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, the chemically modified nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

A modified base of a unnatural nucleic acid includes, but may be not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine, 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base may be replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2′-deoxyadenosine.

In some cases, the at least one chemical modification can comprise chemically modifying the 5′ or 3′ end such as 5′ cap or 3′ tail of the engineered guide RNA. In some embodiments, the engineered guide RNA can comprise a chemical modification comprising 3′ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some embodiments, deaza nucleotides, e.g., 7-deaza-adenosine, can be incorporated into the gRNA. In some embodiments, O- and N-alkylated nucleotides, e.g., N6-methyladenosine, can be incorporated into the gRNA. In some embodiments, sugar-modified ribonucleotides can be incorporated, e.g., wherein the 2′ OH-group may be replaced by a group selected from H,—OR,—R (wherein R can be, e.g., alkyl, cycloalkyl, aryl, aralkyl, heteroaryl or sugar), halo,—SH,—SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, the nucleotides in the overhang region of the gRNA can each independently be a modified or unmodified nucleotide including, but not limited to 2′-sugar modified, such as, 2-F 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), or any combinations thereof.

### Delivery

An engineered guide RNA described herein (e.g., a guide RNA comprising a barbell macro-footprint sequence and at least some elements of a micro-footprint sequence) or a polynucleotide encoding the same can be delivered via a delivery vehicle.

An engineered polynucleotide of the present disclosure (e.g., an engineered polynucleotide encoding for an engineered guide RNA comprising a barbell macro-footprint sequence and at least some elements of a micro-footprint sequence) can be delivered via a delivery vehicle. In some embodiments, the delivery vehicle is a vector. A vector can facilitate delivery of the engineered polynucleotide into a cell to genetically modify the cell. In some examples, the vector comprises DNA, such as double stranded or single stranded DNA. In some examples, the delivery vector can be a eukaryotic vector, a prokaryotic vector (e.g., a bacterial vector or plasmid), a viral vector, or any combination thereof. In some embodiments, the vector is an expression cassette. In some embodiments, a viral vector comprises a viral capsid, an inverted terminal repeat sequence, and the engineered polynucleotide can be used to deliver the engineered guide RNA to a cell.

In some embodiments, the viral vector can be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector can be a recombinant vector, a hybrid vector, a chimeric vector, a self-complementary vector, a single-stranded vector, or any combination thereof.

In some embodiments, the viral vector can be an adeno-associated virus (AAV). In some embodiments, the AAV can be any AAV known in the art. In some embodiments, the viral vector can be of a specific serotype. In some embodiments, the viral vector can be an AAV1 serotype, AAV2 serotype, AAV3 serotype, AAV4 serotype, AAV5 serotype, AAV6 serotype, AAV7 serotype, AAV8 serotype, AAV9 serotype, AAV10 serotype, AAV11 serotype, AAV 12 serotype, AAV13 serotype, AAV14 serotype, AAV15 serotype, AAV16 serotype, AAV.rh8 serotype, AAV.rh10 serotype, AAV.rh20 serotype, AAV.rh39 serotype, AAV.Rh74 serotype, AAV.RHM4-1 serotype, AAV.hu37 serotype, AAV.Anc80 serotype, AAV.Anc80L65 serotype, AAV.7m8 serotype, AAV.PHP.B serotype, AAV2.5 serotype, AAV2tYF serotype, AAV3B serotype, AAV.LK03 serotype, AAV.HSC1 serotype, AAV.HSC2 serotype, AAV.HSC3 serotype, AAV.HSC4 serotype, AAV.HSC5 serotype, AAV.HSC6 serotype, AAV.HSC7 serotype, AAV.HSC8 serotype, AAV.HSC9 serotype, AAV.HSC10 serotype, AAV.HSC11 serotype, AAV.HSC12 serotype, AAV.HSC13 serotype, AAV.HSC14 serotype, AAV.HSC15 serotype, AAV.HSC16 serotype, and AAVhu68 serotype, a derivative of any of these serotypes, or any combination thereof.

In some embodiments, the AAV vector can be a recombinant vector, a hybrid AAV vector, a chimeric AAV vector, a self-complementary AAV (scAAV) vector, a single-stranded AAV, or any combination thereof.

In some embodiments, the AAV vector can be a recombinant AAV (rAAV) vector. Methods of producing recombinant AAV vectors can be known in the art and generally involve, in some cases, introducing into a producer cell line: (1) DNA necessary for AAV replication and synthesis of an AAV capsid, (b) one or more helper constructs comprising the viral functions missing from the AAV vector, (c) a helper virus, and (d) the plasmid construct containing the genome of the AAV vector, e.g., ITRs, promoter and engineered guide RNA sequences, etc. In some examples, the viral vectors described herein can be engineered through synthetic or other suitable means by references to published sequences, such as those that can be available in the literature. For example, the genomic and protein sequences of various serotypes of AAV, as well as the sequences of the native terminal repeats (TRs), Rep proteins, and capsid subunits can be known in the art and can be found in the literature or in public databases such as GenBank or Protein Data Bank (PDB).

In some examples, methods of producing delivery vectors herein comprising packaging an engineered polynucleotide of the present disclosure (e.g., an engineered polynucleotide encoding for an engineered guide RNA comprising a barbell macro-footprint sequence and at least some elements of a micro-footprint sequence) in an AAV vector. In some examples, methods of producing the delivery vectors described herein comprise, (a) introducing into a cell: (i) a polynucleotide comprising a promoter and an engineered guide RNA payload disclosed herein; and (ii) a viral genome comprising a Replication (Rep) gene and Capsid (Cap) gene that encodes a wild-type AAV capsid protein or modified version thereof; (b) expressing in the cell the wild-type AAV capsid protein or modified version thereof; (c) assembling an AAV particle; and (d) packaging the payload disclosed herein in the AAV particle, thereby generating an AAV delivery vector. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site, thereby enabling formation of a self-complementary AAV.

In some examples, a hybrid AAV vector can be produced by transcapsidation, e.g., packaging an inverted terminal repeat (ITR) from a first serotype into a capsid of a second serotype, wherein the first and second serotypes may not be the same. In some examples, the Rep gene and ITR from a first AAV serotype (e.g., AAV2) can be used in a capsid from a second AAV serotype (e.g., AAV5 or AAV9), wherein the first and second AAV serotypes may not be the same. As a non-limiting example, a hybrid AAV serotype comprising the AAV2 ITRs and AAV9 capsid protein can be indicated AAV2/9. In some examples, the hybrid AAV delivery vector comprises an AAV2/1, AAV2/2, AAV 2/4, AAV2/5, AAV2/8, or AAV2/9 vector.

In some examples, the AAV vector can be a chimeric AAV vector. In some examples, the chimeric AAV vector comprises an exogenous amino acid or an amino acid substitution, or capsid proteins from two or more serotypes. In some examples, a chimeric AAV vector can be genetically engineered to increase transduction efficiency, selectivity, or a combination thereof.

In some examples, the AAV vector comprises a self-complementary AAV genome. Self-complementary AAV genomes can be generally known in the art and contain both DNA strands which can anneal together to form double-stranded DNA.

In some examples, the delivery vector can be a retroviral vector. In some examples, the retroviral vector can be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector can be transfected such that the majority of sequences coding for the structural genes of the virus (e.g., gag, pol, and env) can be deleted and replaced by the gene(s) of interest.

In some examples, the delivery vehicle can be a non-viral vector. In some examples, the delivery vehicle can be a plasmid. In some embodiments, the plasmid comprises DNA. In some examples, the plasmid comprises circular double-stranded DNA. In some examples, the plasmid can be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid can be a minicircle plasmid. In some examples, the plasmid contains one or more genes that provide a selective marker to induce a target cell to retain the plasmid. In some examples, the plasmid can be formulated for delivery through injection by a needle carrying syringe. In some examples, the plasmid can be formulated for delivery via electroporation. In some examples, the plasmids can be engineered through synthetic or other suitable means known in the art. For example, in some cases, the genetic elements can be assembled by restriction digest of the desired genetic sequence from a donor plasmid or organism to produce ends of the DNA which can then be readily ligated to another genetic sequence.

In some embodiments, the vector containing the engineered polynucleotide is a non-viral vector system. In some embodiments, the non-viral vector system comprises cationic lipids, or polymers. For example, the non-viral vector system comprises can be a liposome or polymeric nanoparticle. In some embodiments, the engineered polynucleotide or a non-viral vector comprising the engineered polynucleotide is delivered to a cell by hydrodynamic injection or ultrasound.

### Methods of Treatment

Disclosed herein are methods of delivering an engineered guide RNA disclosed herein (e.g., an engineered guide RNA comprising a barbell macro-footprint sequence and at least some elements of a micro-footprint sequence), or a vector encoding an engineered guide RNA disclosed herein, to a cell or to a subject in need thereof. The engineered guide RNA described here, upon hybridization to a target RNA forms a guide-target RNA scaffold, where the engineered guide RNA comprises a barbell macro-footprint sequence and at least some elements of a micro-footprint sequence. An engineered guide RNA described here can hybridize to a target RNA that is associated with or implicated in a disease or condition, which can cause, or partially cause, or contribute to one or more symptoms of the associated disease or condition or can be associated with or implicated in a pathway that manifests in the disease or condition. In some embodiments, engineered guide RNAs of the disclosure comprising a barbell macro-footprint sequence and at least some elements of a micro-footprint sequence can be used to treat a disease or condition. The disease or condition can be selected from the group consisting of: a neurodegenerative disease or disorder, a muscular disease or disorder, a metabolic disease or disorder, an ocular disease or disorder, a liver disease or disorder, a cancer, and any combination thereof. In some aspects, the disease or condition can be selected from the group consisting of: Rett syndrome, Huntington's disease, Parkinson's disease, Alzheimer's disease, a muscular dystrophy, Tay-Sachs disease, and any combination thereof.

In some embodiments, the described engineered guide RNAs of the disclosure comprising a barbell macro-footprint sequence and at least some elements of a micro-footprint sequence can hybridize to target RNAs to form guide-target RNA scaffolds, where the target RNA can be selected from the group consisting of: ABCA4, ALAS1, APP, ATP7B, CFTR, DMD, DMPK, DUX4, GAPDH, GBA, HEXA, HFE, LIPA, LRRK2, MAPT, PCSK9 start site, PINK1, PMP22, SERPINA1, SCNN1A start site, SNCA, or SOD1, a fragment of any one of these, and any combination thereof. In some embodiments, the engineered guide RNAs of the disclosure comprising a barbell macro-footprint sequence and at least some elements of a micro-footprint sequence, where the engineered guide RNAs target any gene of interest in need of editing, and compositions comprising such engineered guide RNAs of the disclosure, can be useful in treating diseases or conditions associated with any of the target RNAs of interest.

Other embodiments of the disclosure can provide for a method of treating a disease or condition in a subject, the method comprising: administering to the subject an effective amount of any of the disclosed engineered guide RNAs comprising a barbell macro-footprint sequence and at least some elements of a micro-footprint sequence, to treat the disease or condition in the subject in need thereof. In some examples, a method of treating a disease or condition in a subject is provided, where the method comprises administering to the subject an effective amount of any of the disclosed polynucleotides encoding any of the engineered guide RNAs of the disclosure comprising a barbell macro-footprint sequence and at least some elements of a micro-footprint sequence, to treat the disease or condition in the subject in need thereof. Further aspects provide for a method of treating a disease or condition in a subject, where the method comprises: administering to the subject an effective amount of any of the disclosed delivery vehicles comprising engineered guide RNAs comprising a barbell macro-footprint sequence and at least some elements of a micro-footprint sequence, to treat the disease or condition in the subject in need thereof. Some examples provide methods of treating a disease or condition in a subject comprising administering to the subject, an effective amount of any of the disclosed delivery vehicles comprising polynucleotides encoding engineered guide RNAs described here comprising a barbell macro-footprint sequence and at least some elements of a micro-footprint sequence, to treat the disease or condition in the subject in need thereof. In further aspects provided here are methods of treating a disease or condition in a subject, where the method comprises: administering to the subject an effective amount of any of the disclosed pharmaceutical compositions comprising engineered guide RNAs comprising a barbell macro-footprint sequence and at least some elements of a micro-footprint sequence, to treat the disease or condition in the subject in need thereof. Some embodiments provide methods of treating a disease or condition in a subject, where the method comprises: administering to the subject an effective amount of any of the disclosed pharmaceutical compositions comprising polynucleotides encoding engineered guide RNAs comprising a barbell macro-footprint sequence and at least some elements of a micro-footprint sequence, to treat the disease or condition in the subject in need thereof. In further examples, methods of treating a disease or condition in a subject comprising administering to the subject, an effective amount of any of the disclosed pharmaceutical compositions comprising delivery vehicles comprising any of the described engineered guide RNAs comprising a barbell macro-footprint sequence and at least some elements of a micro-footprint sequence or polynucleotides encoding engineered guide RNAs disclosed here, to treat the disease or condition in the subject in need thereof.

In some aspects, provided here are uses of any of the described engineered guide RNAs comprising a barbell macro-footprint sequence and at least some elements of a micro-footprint sequence for the preparation of a medicament for the treatment of any of the diseases or conditions disclosed here. Some embodiments provide uses of any of the described polynucleotides encoding any of the engineered guide RNAs comprising a barbell macro-footprint sequence and at least some elements of a micro-footprint sequence for the preparation of a medicament for the treatment of any of the diseases or conditions disclosed here. In other examples, provided here are uses of any of the described delivery vehicles containing engineered guide RNAs comprising a barbell macro-footprint sequence and at least some elements of a micro-footprint sequence for the preparation of a medicament for the treatment of any of the diseases or conditions disclosed here. Further aspects provide uses of any of the described delivery vehicles containing polynucleotides encoding engineered guide RNAs comprising a barbell macro-footprint sequence and at least some elements of a micro-footprint sequence described here for the preparation of a medicament for the treatment of any of the diseases or conditions disclosed here. In some examples, provided here are uses of any of the described pharmaceutical compositions comprising any of the engineered guide RNAs comprising a barbell macro-footprint sequence and at least some elements of a micro-footprint sequence for the preparation of a medicament for the treatment of any of the diseases or conditions disclosed here. Additional examples provide uses of any of the described pharmaceutical compositions polynucleotides encoding engineered guide RNAs comprising a barbell macro-footprint sequence and at least some elements of a micro-footprint sequence for the preparation of a medicament for the treatment of any of the diseases or conditions disclosed here. In other aspects, provided here are uses of any of the described pharmaceutical compositions comprising delivery vehicles comprising engineered guide RNAs comprising a barbell macro-footprint sequence and at least some elements of a micro-footprint sequence or polynucleotides encoding engineered guide RNAs described here for the preparation of a medicament for the treatment of any of the diseases or conditions disclosed here.

Other embodiments can provide any of the described engineered guide RNAs comprising a barbell macro-footprint sequence and at least some elements of a micro-footprint sequence for use in treating any of the disclosed diseases or conditions. In some aspects, any of the described polynucleotides encoding engineered guide RNAs of the disclosure comprising a barbell macro-footprint sequence and at least some elements of a micro-footprint sequence, can be used in treating any of the diseases or conditions disclosed here. Additional examples provide any of the described delivery vehicles comprising engineered guide RNAs of the disclosure comprising a barbell macro-footprint sequence and at least some elements of a micro-footprint sequence or polynucleotides encoding any of the engineered guide RNAs described here, for use in treating any of the disclosed diseases or conditions. In some embodiments, provided here are pharmaceutical compositions comprising engineered guide RNAs of the disclosure comprising a barbell macro-footprint sequence and at least some elements of a micro-footprint sequence, for use in treating any of the diseases or conditions disclosed here. Other aspects provide pharmaceutical compositions disclosed here comprising polynucleotides encoding engineered guide RNAs of the disclosure comprising a barbell macro-footprint sequence and at least some elements of a micro-footprint sequence, for use in treating any of the disclosed diseases or conditions. In further examples, provided here are pharmaceutical compositions of the disclosure comprising any of the delivery vehicles comprising any of the described engineered guide RNAs comprising a barbell macro-footprint sequence and at least some elements of a micro-footprint or any of the described polynucleotides encoding any of the engineered guide RNAs described here, for use in treating any of the disclosed diseases or conditions. in a subject.

Engineered guide RNA medicaments described here can be used to treat a disease or condition in a subject in need thereof. Other examples of the disclosure provide for any of the described engineered guide RNAs comprising a barbell macro-footprint sequence and at least some elements of a micro-footprint sequence for use in a treatment of a disease or condition in a subject described here. In some embodiments, the uses and methods disclosed here can be directed to treating a disease or condition, which can also include preventing a disease or condition, one or more symptoms of the disease or condition, a pathway or component of a pathway that manifests in the disease or condition, or any combination thereof. In other embodiments, the uses and methods disclosed here can treat a disease or condition, which can also include treating a disease or condition, one or more symptoms of the disease or condition, a pathway or component of a pathway that manifests in the disease or condition, or any combination thereof. In some embodiments, any of the uses and methods described here can be for treating (including preventing, ameliorating) a disease or condition selected from the group consisting of: a neurodegenerative disease or disorder, a muscular disease or disorder, a metabolic disease or disorder, an ocular disease or disorder, a liver disease or disorder, a cancer, and any combination thereof. Other diseases or conditions of the disclosure can be selected from the group consisting of: Duchenne's Muscular Dystrophy (DMD), Becker muscular dystrophy, myotonic dystrophy, Facioscapulohumeral muscular dystrophy, Rett's syndrome, Charcot-Marie-Tooth disease, Alzheimer's disease, a tauopathy, Parkinson's disease, alpha-1 antitrypsin deficiency, cystic fibrosis-like disease, Wilson disease, Stargardt disease, and any combination thereof.

In some methods of the disclosure for treatment of a disease or condition with the disclosed engineered guide RNAs comprising a barbell macro-footprint sequence and at least some elements of a micro-footprint sequence of the description, the disease or condition can be associated with a mutation in a gene, or RNA encoded by the gene, or the mutation is introduced into a gene. Other examples provide methods of the disclosure for treatment of a disease or condition with any of the described polynucleotides encoding engineered guide RNAs of the description, where the disease or condition can be associated with a mutation in a gene, or RNA encoded by the gene, or the mutation is introduced into a gene. In further aspects, methods of the disclosure for treatment of a disease or condition with the disclosed delivery vehicles comprising any of the engineered guide RNAs comprising a barbell macro-footprint sequence and at least some elements of a micro-footprint sequence of the description or polynucleotides encoding the engineered guide RNAs, where the disease or condition can be associated with a mutation in a gene, or RNA encoded by the gene, or the mutation is introduced into a gene. Some embodiments provide methods of the disclosure for treatment of a disease or condition with any of the described pharmaceutical compositions comprising engineered guide RNAs comprising a barbell macro-footprint sequence and at least some elements of a micro-footprint sequence of the description, where the disease or condition can be associated with a mutation in a gene, or RNA encoded by the gene, or the mutation is introduced into a gene. Other aspects provide methods for treatment of a disease or condition with any of the described pharmaceutical compositions comprising any of the polynucleotides encoding engineered guide RNAs of the disclosure, where the disease or condition can be associated with a mutation in a gene, or RNA encoded by the gene, or the mutation is introduced into a gene. Further examples provide methods for treatment of a disease or condition with any of the described pharmaceutical compositions comprising any of the disclosed delivery vehicles described here comprising any of the engineered guide RNAs of the disclosure, where the disease or condition can be associated with a mutation in a gene, or RNA encoded by the gene, or the mutation is introduced into a gene. Yet other embodiments provide methods for treatment of a disease or condition with any of the described pharmaceutical compositions comprising any of the disclosed delivery vehicles described here comprising any of the polynucleotides encoding any of the engineered guide RNAs of the disclosure, where the disease or condition can be associated with a mutation in a gene, or RNA encoded by the gene, or the mutation is introduced into a gene. Additional aspects provide a gene selected from the group consisting of: ABCA4, ALAS1, APP, ATP7B, ATP7B G1226R, CFTR, DMD, DMPK, DUX4, GAPDH, GBA, HEXA, HFE, HFE C282Y, LIPA, LIPA c.894 G>A, LRRK2, MAPT, PCSK9 start site, PINK1, PMP22, SERPINA1, SERPINA1 E342K, SCNN1A start site, SNCA, SOD1, a fragment of any of these, and any combination thereof. In some embodiments, an engineered guide RNA of the present disclosure comprising a barbell macro-footprint can target an IDUA mRNA. In some embodiments, an engineered guide RNA of the present disclosure comprising a barbell macro-footprint may not target an IDUA mRNA.

In various embodiments described here, engineered guide RNAs of the disclosure comprising a barbell macro-footprint sequence and at least some elements of a micro-footprint sequence can be useful as therapeutics for treating subjects suffering from a disease or condition, where the subject can have a target RNA comprising a mutation or a target RNA in need of a mutation. Some examples described here are directed to polynucleotides encoding engineered guide RNAs of the disclosure comprising a barbell macro-footprint sequence and at least some elements of a micro-footprint sequence that can be useful as therapeutics for treating subjects suffering from a disease or condition, where the subject can have a target RNA comprising a mutation or a target RNA in need of a mutation. In other aspects described here, any of the described delivery vehicles comprising engineered guide RNAs of the disclosure comprising a barbell macro-footprint sequence and at least some elements of a micro-footprint sequence can be useful as therapeutics for treating subjects suffering from a disease or condition, where the subject can have a target RNA comprising a mutation or a target RNA in need of a mutation. Further embodiments described here are directed to delivery vehicles comprising polynucleotides encoding any of the engineered guide RNAs of the disclosure comprising a barbell macro-footprint sequence and at least some elements of a micro-footprint sequence that can be useful as therapeutics for treating subjects suffering from a disease or condition, where the subject can have a target RNA comprising a mutation or a target RNA in need of a mutation. In some examples described here, any of the described pharmaceutical compositions comprising engineered guide RNAs of the disclosure comprising a barbell macro-footprint sequence and at least some elements of a micro-footprint sequence can be useful as therapeutics for treating subjects suffering from a disease or condition, where the subject can have a target RNA comprising a mutation or a target RNA in need of a mutation. Additional aspects described here are directed to pharmaceutical compositions comprising delivery vehicles with any of the disclosed engineered guide RNAs or polynucleotides encoding engineered guide RNAs of the disclosure comprising a barbell macro-footprint sequence and at least some elements of a micro-footprint sequence that can be useful as therapeutics for treating subjects suffering from a disease or condition, where the subject can have a target RNA comprising a mutation or a target RNA in need of a mutation.

Further embodiments provide a therapeutic comprising any of the disclosed engineered guide RNAs comprising a barbell macro-footprint sequence and at least some elements of a micro-footprint sequence or any of the described polynucleotides encoding any of the disclosed engineered guide RNAs. In other aspects, a therapeutic described here comprises a delivery vehicle comprising any of the disclosed engineered guide RNAs comprising a barbell macro-footprint sequence and at least some elements of a micro-footprint sequence or any of the described polynucleotides encoding any of the disclosed engineered guide RNAs. Additional embodiments provide a therapeutic comprising a pharmaceutical composition comprising any of the disclosed engineered guide RNAs comprising a barbell macro-footprint sequence and at least some elements of a micro-footprint sequence, any of the described polynucleotides encoding any of the disclosed engineered guide RNAs, or any of the described delivery vehicles. In some examples of the disclosure, a method of treating (including preventing, reducing, or ameliorating) a subject suffering from a disease or a condition or symptoms of the disease or the condition, comprises administering to the subject, a therapeutic that facilitates editing of a target RNA. In some embodiments, editing the target RNA can facilitate correction of a mutation. The mutation can be a missense mutation or a nonsense mutation. In some embodiments, the RNA editing can involve introducing mutations into a target RNA of interest. In some embodiments, the engineered guide RNAs of the present disclosure can facilitate multiple RNA edits of a target RNA.

In some embodiments of the disclosure, target RNAs of the guide-target RNA scaffold formed upon hybridization of an engineered guide RNA described here and a target RNA, can have a mutation, or a mutation can be introduced into the target RNAs using the engineered guide RNAs of the disclosure.

ABCA4. Some examples provide engineered RNAs of the disclosure comprising a barbell macro-footprint sequence and at least some elements of a micro-footprint, which can comprise a targeting sequence with target complementarity to a target RNA that is an ATP binding cassette subfamily A member 4 (ABCA4). In some embodiments of the disclosure, the therapeutics or engineered guide RNAs described here comprising a barbell macro-footprint sequence and at least some elements of a micro-footprint sequence, can facilitate RNA editing of an ABCA4 target RNA, which can have a mutation selected from the group consisting of: G6320A; G5714A; G5882A; and any combination thereof. The engineered guide RNAs described here comprising a barbell macro-footprint sequence and at least some elements of a micro-footprint sequence can facilitate a correction of the G to A mutations of the ABCA4 gene. In some examples, the ABCA4 mutation causes or contributes to macular degeneration in a subject in need thereof to whom the described engineered guide RNA can be administered for treatment. In some examples, the macular degeneration can be Stargardt macular degeneration. In some embodiments, the human subject can be at risk of developing or has developed Stargardt macular degeneration (or Stargardt disease), which could be caused, at least in part, by one of the indicated mutations of ABCA4. Some embodiments of the disclosure provide for engineered guide RNAs comprising a barbell macro-footprint sequence and at least some elements of a micro-footprint sequence, for facilitating editing thereby correcting the mutation in ABCA4 and reducing the incidence of Stargardt disease in the subject. In some examples the target RNA molecule comprises an adenosine with a 5′ G. In some examples, the adenosine with the 5′ G can be the base intended for chemical modification by the RNA editing entity. In some examples, the RNA editing entity can be an ADAR, and the ADAR chemically modifies the adenosine with the 5′ G after recruitment by the guide-target RNA scaffold. Accordingly, such engineered guide RNAs can be used in methods of treating a subject suffering from Stargardt macular degeneration.

A guide RNA targeting ABCA4 can comprise a first and second internal loop positioned with respect to the base that is most proximal to the A/C mismatch in the guide-target RNA complex. In some embodiments, the first internal loop is positioned from about 5 bases away from the A/C mismatch to about 15 bases away from the A/C mismatch with respect to the base of the first internal loop that is most proximal to the A/C mismatch. In some embodiments, the first internal loop is positioned 15 bases away from the A/C mismatch with respect to the base of the first internal loop that is most proximal to the A/C mismatch. In some embodiments, the second internal loop is positioned from about 12 bases away from the A/C mismatch to about 40 bases away from the A/C mismatch with respect to the base of the second internal loop that is most proximal to the A/C mismatch. In some embodiments, the second internal loop is positioned 33 bases away from the A/C mismatch with respect to the base of the second internal loop that is most proximal to the A/C mismatch.

An engineered guide RNA targeting ABCA4 can comprise a polynucleotide sequence with at least 80%, 85%, 90%, 92%, 95%, 97%, or 99% sequence identity to any one of SEQ ID NO: 1-105, 2729-2761, or 2772-2843.

APP. Other examples of the disclosure can be directed to engineered guide RNAs comprising a barbell macro-footprint sequence and at least some elements of a micro-footprint sequence, where the engineered guide RNAs hybridize to target RNA that is amyloid precursor protein (APP), which can be targeted for editing. In some embodiments, a specific residue can be targeted utilizing the engineered guide RNAs comprising a barbell macro-footprint sequence and at least some elements of a micro-footprint sequence and methods described here. In some examples, the engineered guide RNAs described here are configured to facilitate an edit of a base of a nucleotide of the target RNA by an RNA editing entity forming an edited target RNA, such that a protein translated from the edited target RNA comprises at least one alteration or mutation selected from the group consisting of: K670E, K670R, K670G, M671V, A673V, A673T, D672G, E682G, H684R, K687R, K687E, K687G, I712X, T714X, and any combination thereof. In some embodiments of the disclosure, the target RNA encodes for an unmodified APP polypeptide that comprises at least one amino acid residue difference as compared to a modified APP polypeptide generated from editing a base of a nucleotide of the APP target RNA, where the at least one amino acid residue difference is selected from the group consisting of: K670E, K670R, K670G, M671V, A673V, A673T, D672G, E682G, H684R, K687R, K687E, K687G, I712X, T714X of the APP polypeptide, and any combination thereof. In some examples, the target RNA molecule encodes, at least in part, an amyloid precursor protein (APP); an APP start site; an APP cleavage site; or a beta secretase (BACE) or gamma secretase cleavage site of an APP protein. In some examples, cleavage of the APP protein at the cleavage site causes or contributes to Amyloid beta (A3 or Abeta) peptide deposition in the brain or blood vessels. In some examples, the Abeta deposition causes or contributes to a neurodegenerative disease. In some examples, the disease comprises Alzheimer's disease, Parkinson's disease, corticobasal degeneration, dementia with Lewy bodies, Lewy body variant of Alzheimer's disease, Parkinson's disease with dementia, Pick's disease, progressive supranuclear palsy, dementia, fronto-temporal dementia with Parkinsonism linked to tau mutations on chromosome 17, or any combination thereof. The engineered guide RNAs of the disclosure comprising a targeting sequence having substantial complementarity to an APP target RNA, where the engineered guide RNAs comprising a barbell macro-footprint sequence and at least some elements of a micro-footprint sequence can be used to facilitate an edit of a base of a nucleotide of the APP target RNA by an RNA editing entity forming an edited APP target RNA, such that a protein translated from the edited APP target RNA comprises at least one alteration or mutation described here. Accordingly, such engineered guide RNAs can be used in methods of treating a subject suffering from a neurodegenerative disease, such as but not limited to, Alzheimer's disease, Parkinson's disease, dementia, and the like.

A guide RNA targeting APP can comprise a first and second internal loop positioned with respect to the base that is most proximal to the A/C mismatch in the guide-target RNA complex. In some embodiments, the first internal loop is positioned from about 5 bases away from the A/C mismatch to about 20 bases away from the A/C mismatch with respect to the base of the first internal loop that is most proximal to the A/C mismatch. In some embodiments, the first internal loop is positioned 10 bases away from the A/C mismatch with respect to the base of the first internal loop that is most proximal to the A/C mismatch. In some embodiments, the second internal loop is positioned from about 15 bases away from the A/C mismatch to about 40 bases away from the A/C mismatch with respect to the base of the second internal loop that is most proximal to the A/C mismatch. In some embodiments, the second internal loop is positioned 33 bases away from the A/C mismatch with respect to the base of the second internal loop that is most proximal to the A/C mismatch.

An engineered guide RNA targeting APP can comprise a polynucleotide sequence with at least 80%, 85%, 90%, 92%, 95%, 97%, or 99% sequence identity to any one of SEQ ID NO: 112-114.

DMPK. In some embodiments, the present disclosure provides engineered guide RNAs comprising a targeting sequence sufficiently complementary to a DMPK target RNA, and a barbell macro-footprint sequence and at least some elements of a micro-footprint sequence, that facilitate RNA editing of DMPK to knockdown expression of myotonic dystrophy protein kinase. Myotonic dystrophy (DM1) is a rare neuromuscular disease characterized by progressive muscular weakness and an inability to relax muscles (myotonia), predominantly distal skeletal muscles. In some embodiments, the engineered guide RNAs comprising a barbell macro-footprint sequence and at least some elements of a micro-footprint sequence, which targets DMPK and compositions comprising such engineered guide RNAs facilitate ADAR-mediated RNA editing of DMPK to knockdown expression of myotonic dystrophy protein kinase.

DUX4. The present disclosure provides engineered guide RNAs comprising a barbell macro-footprint sequence and at least some elements of a micro-footprint sequence, and a targeting sequence sufficiently complementary to a DUX4 target RNA that facilitates RNA editing DUX4 to knockdown expression of DUX4 protein. Facioscapulohumeral muscular dystrophy (FSHD), an autosomal dominant neuromuscular disorder, is a rare neuromuscular disease characterized by progressive skeletal muscle weakness with significant heterogeneity in phenotypic severity and age of onset. Genetic causes of FSHD include mutations in the D4Z4 repeat region on chromosome 4 that lead to hypomethylation and dysregulated expression of the DUX4 gene (a germline transcription factor). In some embodiments, the present disclosure provides compositions of engineered guide RNAs that target DUX4 and comprise a barbell macro-footprint sequence and at least some elements of a micro-footprint sequence and facilitate ADAR-mediated RNA editing of DUX4, specifically, DUX4-FL to mediate DUX4-FL knockdown.

In some embodiments, the engineered guide RNAs of the present disclosure comprising a barbell macro-footprint sequence and at least some elements of a micro-footprint sequence facilitate ADAR-mediated RNA editing of target genes (e.g., DMPK, DUX4-FL), which results in knockdown of protein levels. The knockdown in protein levels is quantitated as a reduction in expression of the protein (e.g., DMPK protein: myotonic dystrophy protein kinase; DUX4-FL protein). The engineered guide RNAs of the present disclosure comprising a barbell macro-footprint sequence and at least some elements of a micro-footprint sequence and a targeting sequence sufficiently complementary to, for example, a DMPK or DUX4 target of interest, can facilitate from 1% to 100% DMPK protein knockdown or DUX4-FL protein knockdown. The engineered RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% DMPK protein knockdown or DUX4-FL protein knockdown. In some embodiments, the engineered RNAs of the present disclosure comprising a barbell macro-footprint sequence and at least some elements of a micro-footprint sequence facilitate from 30% to 60% DMPK protein knockdown or DUX4-FL protein knockdown. DMPK protein knockdown or DUX4-FL protein knockdown can be measured by an assay comparing a sample or subject treated with the engineered guide RNA of the disclosure comprising a barbell macro-footprint sequence and at least some elements of a micro-footprint sequence to a control sample or subject not treated with the engineered guide RNA comprising a barbell macro-footprint sequence and at least some elements of a micro-footprint sequence.

A guide RNA targeting DUX4 can comprise a first and second internal loop positioned with respect to the base that is most proximal to the A/C mismatch in the guide-target RNA complex. In some embodiments, the first internal loop is positioned from about 7 bases away from the A/C mismatch to about 30 bases away from the A/C mismatch with respect to the base of the first internal loop that is most proximal to the A/C mismatch. In some embodiments, the first internal loop is positioned 10 bases away from the A/C mismatch with respect to the base of the first internal loop that is most proximal to the A/C mismatch. In some embodiments, the second internal loop is positioned from about 18 bases away from the A/C mismatch to about 34 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch.

An engineered guide RNA targeting DUX4 can comprise a polynucleotide sequence with at least 80%, 85%, 90%, 92%, 95%, 97%, or 99% sequence identity to any one of SEQ ID NO: 118-167, 2686-2728, 2769-2771, 2844-3078, or 3081-3082.

GRN. A guide RNA targeting GRN can comprise a first and second internal loop positioned with respect to the base that is most proximal to the A/C mismatch in the guide-target RNA complex. In some embodiments, the first internal loop is positioned from about 5 bases away from the A/C mismatch to about 20 bases away from the A/C mismatch with respect to the base of the first internal loop that is most proximal to the A/C mismatch. In some embodiments, the first internal loop is positioned 12 bases away from the A/C mismatch with respect to the base of the first internal loop that is most proximal to the A/C mismatch. In some embodiments, the second internal loop is positioned from about 18 bases away from the A/C mismatch to about 38 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch.

LRRK2. The described engineered guide RNAs of the disclosure can comprise a targeting sequence with target complementarity to a leucine-rich repeat kinase 2 (LRRK2) target RNA, further comprising a barbell macro-footprint sequence and at least some elements of a micro-footprint sequence. In some embodiments, such engineered guide RNAs can facilitate RNA editing of LRRK2 encoded mutations associated with a disease or condition, where a LRRK2 encoded mutation can be selected from the group consisting of: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q1111H, 11122V, A1151T, L1165P, 11192V, H1216R, S1228T, P1262A, R1325Q, I1371V, R1398H, T1410M, D1420N, N1437H, R1441C, R1441G, R1441H, A1442P, P1446L, V1450I, K1468E, R1483Q, R1514Q, P1542S, V1613A, R₁₆₂₈P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, Q2490N, and any combination thereof. In some embodiments, such engineered guide RNAs that target LRRK2 and comprising a barbell macro-footprint sequence and at least some elements of a micro-footprint sequence can be used for treating a disease or condition such as a neurodegenerative disease (Parkinson's) by producing an edit, a knockdown or both of a pathogenic variant of LRRK2. In some aspects, a pathogenic variant of LRRK2 can comprise a G2019S mutation. The engineered guide RNAs targeting LRRK2 and comprising a barbell macro-footprint sequence and at least some elements of a micro-footprint sequence can be used to treat a LRRK2 associated disease or condition such as but not limited to a muscular dystrophy, an ornithine transcarbamylase deficiency, a retinitis pigmentosa, a breast cancer, an ovarian cancer, Alzheimer's disease, pain, Stargardt macular dystrophy, Charcot-Marie-Tooth disease, Rett syndrome, or any combination thereof.

A guide RNA targeting LRRK2 can comprise a first and second internal loop positioned with respect to the base that is most proximal to the A/C mismatch in the guide-target RNA complex. In some embodiments, the first internal loop is positioned from about 7 bases away from the A/C mismatch to about 30 bases away from the A/C mismatch with respect to the base of the first internal loop that is most proximal to the A/C mismatch. In some embodiments, the first internal loop is positioned 10 bases away from the A/C mismatch with respect to the base of the first internal loop that is most proximal to the A/C mismatch. In some embodiments, the second internal loop is positioned from about 18 bases away from the A/C mismatch to about 34 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch.

An engineered guide RNA targeting LRRK2 can comprise a polynucleotide sequence with at least 80%, 85%, 90%, 92%, 95%, 97%, or 99% sequence identity to any one of SEQ ID NO: 118-167, 2686-2728, 2769-2771, 2844-3078, or 3081-3082.

PMP22. The present disclosure provides for engineered guide RNAs targeting PMP22 and comprising a barbell macro-footprint sequence and at least some elements of a micro-footprint sequence that facilitate RNA editing of PMP22 to knockdown expression of peripheral myelin protein-22 (PMP22). Charcot-Marie-Tooth Syndrome (CMT1A) is the most common genetically-driven peripheral neuropathy, characterized by progressive distal muscle atrophy, sensory loss and foot/hand deformities. In some embodiments, the present disclosure provides compositions of engineered guide RNAs that target PMP22 and comprising a barbell macro-footprint sequence and at least some elements of a micro-footprint sequence, which facilitate ADAR-mediated RNA editing of PMP22. In some embodiments, the engineered guide RNAs of the present disclosure target a coding sequence in PMP22. For example, the coding sequence can be a translation initiation site (TIS) (AUG) of PMP22 and the engineered guide RNAs disclosed here can facilitate ADAR-mediated RNA editing of AUG to GUG. The engineered guide RNAs of the present disclosure that target PMP22 and comprise a barbell macro-footprint sequence and at least some elements of a micro-footprint sequence can facilitate ADAR-mediated RNA editing of PMP22, thereby, effecting its protein knockdown.

SERPINA1. In some embodiments, the disclosure is directed to an engineered guide RNA comprising a barbell macro-footprint sequence and at least some elements of a micro-footprint sequence and a targeting sequence substantially complementary to the serpin family A member 1 (SERPINA1) target RNA, where the engineered guide RNA can facilitate RNA editing of SERPINA1. For example, such engineered guide RNAs can facilitate an ADAR-mediated correction of a G to A mutation at nucleotide position 9989 of a SERPINA1 gene (G9989A) or the SERPINA1 target RNA encodes a mutation of E342K. In some examples, the mutation causes or contributes to an antitrypsin (AAT) deficiency, such as alpha-1 antitrypsin deficiency (AATD) in a subject to whom the engineered guide RNA of the disclosure comprising a barbell macro-footprint sequence and at least some elements of a micro-footprint sequence can be administered. Some embodiments are directed to methods of treating a subject who can be human and at risk of developing or has developed alpha-1 antitrypsin deficiency. Such alpha-1 antitrypsin deficiency can be at least partially caused by a mutation of SERPINA1, for which an engineered guide RNA comprising a barbell macro-footprint sequence and at least some elements of a micro-footprint sequence described here can facilitate editing of the mutation in, for example, a human subject, thus correcting the mutation in SERPINA1 and reducing the incidence of alpha-1 antitrypsin deficiency in the subject. Accordingly, the engineered guide RNAs of the present disclosure targeting SERPINA1 and comprising a barbell macro-footprint sequence and at least some elements of a micro-footprint sequence can be used in a method of treating a subject suffering from an alpha-1 antitrypsin deficiency.

Some aspects provide engineered guide RNAs comprising exemplary targeting sequences that can target a SERPINA1 gene linked to any promoter (e.g., U1, U6, U7) disclosed herein that can be incorporated to drive expression of the engineered guide RNAs comprising a barbell macro-footprint sequence and at least some elements of a micro-footprint sequence. Alpha-1 antitrypsin deficiency can be at least partially caused by a mutation of SERPINA1, for which the engineered guide RNA described here can facilitate editing in, thus correcting the mutation in SERPINA1 and reducing the incidence of alpha-1 antitrypsin deficiency in the subject.

A guide RNA targeting SERPINA1 can comprise a first and second internal loop positioned with respect to the base that is most proximal to the A/C mismatch in the guide-target RNA complex. In some embodiments, the first internal loop is positioned from about 5 bases away from the A/C mismatch to about 20 bases away from the A/C mismatch with respect to the base of the first internal loop that is most proximal to the A/C mismatch. In some embodiments, the first internal loop is positioned 12 bases away from the A/C mismatch with respect to the base of the first internal loop that is most proximal to the A/C mismatch. In some embodiments, the second internal loop is positioned from about 12 bases away from the A/C mismatch to about 40 bases away from the A/C mismatch with respect to the base of the second internal loop that is most proximal to the A/C mismatch. In some embodiments, the second internal loop is positioned 24 bases away from the A/C mismatch with respect to the base of the second internal loop that is most proximal to the A/C mismatch

An engineered guide RNA targeting SERPINA1 can comprise a polynucleotide sequence with at least 80%, 85%, 90%, 92%, 95%, 97%, or 99% sequence identity to any one of SEQ ID NO: 2762-2768 or 3083-3086.

SNCA. In some embodiments, the present disclosure provides engineered guide RNAs, compositions, and methods of using the engineered guide RNAs comprising a barbell macro-footprint sequence and at least some elements of a micro-footprint sequence that can facilitate RNA editing of SNCA. In some embodiments, such engineered guide RNAs described here comprising a barbell macro-footprint sequence and at least some elements of a micro-footprint sequence can knock down expression of SNCA, for example, by facilitating editing at a 3′ UTR of an SNCA gene. Such engineered guide RNAs comprising a barbell macro-footprint sequence and at least some elements of a micro-footprint sequence targeting a site in SNCA can be encoded for by an engineered polynucleotide construct of the present disclosure. In some embodiments of the disclosure, the engineered guide RNAs described here comprising a targeting sequence with target complementarity to an alpha-synuclein (SNCA) target RNA, and comprising a barbell macro-footprint sequence and at least some elements of a micro-footprint sequence, can be used in the treatment of neurodegenerative disease, including but not limited to Alzheimer's disease or other diseases associated with an accumulation of Tau-p. In some examples, the engineered RNA comprises an engineered guide RNA, which targets an SNCA start codon.

In some examples, polymorphisms in either LRRK2 (G2019S) or SNCA can be associated with an increased risk of idiopathic Parkinson's Disease, and the disease or condition can comprise idiopathic Parkinson's Disease. In some examples, administration of the engineered RNAs disclosed herein comprising a barbell macro-footprint sequence and at least some elements of a micro-footprint sequence can edit LRRK2 G2019S (G>A conversion at the 6055th nucleotide) and edit the start codon SNCA by editing any of the nucleotides of the ATG to decrease the expression of SNCA. Some examples of the disclosure provide for engineered guide RNAs comprising a targeting sequence with sufficient complementarity to an SNCA target RNA and comprising a barbell macro-footprint sequence and at least some elements of a micro-footprint sequence, where the SNCA comprises a mutation for RNA editing selected from the group consisting of: a translation initiation site (TIS) AUG to GTG in Codon 1, a TIS AUG in Codon 5, an AUG at position 265 in Exon 2, and any combination thereof.

A guide RNA targeting SNCA can comprise a first and second internal loop positioned with respect to the base that is most proximal to the A/C mismatch in the guide-target RNA complex. In some embodiments, the first internal loop is positioned from about 6 bases away from the A/C mismatch to about 20 bases away from the A/C mismatch with respect to the base of the first internal loop that is most proximal to the A/C mismatch. In some embodiments, the first internal loop is positioned 6 bases away from the A/C mismatch with respect to the base of the first internal loop that is most proximal to the A/C mismatch. In some embodiments, the second internal loop is positioned from about 15 bases away from the A/C mismatch to about 38 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch

An engineered guide RNA targeting SNCA can comprise a polynucleotide sequence with at least 80%, 85%, 90%, 92%, 95%, 97%, or 99% sequence identity to any one of SEQ ID NO: 2480-2681.

MAPT. A guide RNA targeting MAPT can comprise a first and second internal loop positioned with respect to the base that is most proximal to the A/C mismatch in the guide-target RNA complex. In some embodiments, the first internal loop is positioned from about 5 bases away from the A/C mismatch to about 15 bases away from the A/C mismatch with respect to the base of the first internal loop that is most proximal to the A/C mismatch. In some embodiments, the first internal loop is positioned 15 bases away from the A/C mismatch with respect to the base of the first internal loop that is most proximal to the A/C mismatch. In some embodiments, the second internal loop is positioned from about 12 bases away from the A/C mismatch to about 40 bases away from the A/C mismatch with respect to the base of the second internal loop that is most proximal to the A/C mismatch. In some embodiments, the second internal loop is positioned 33 bases away from the A/C mismatch with respect to the base of the second internal loop that is most proximal to the A/C mismatch.

An engineered guide RNA targeting MAPT can comprise a polynucleotide sequence with at least 80%, 85%, 90%, 92%, 95%, 97%, or 99% sequence identity to any one of SEQ ID NO: 115-117, 1519-2479 or 2682-2685.

SOD1. The present disclosure provides for engineered guide RNAs comprising a barbell macro-footprint sequence and at least some elements of a micro-footprint sequence that facilitate RNA editing of SOD1 to knockdown expression of the superoxide dismutase enzyme. Amyotrophic lateral sclerosis (ALS) is a rapidly progressing neurodegenerative disease characterized by death of motor neurons and loss of voluntary muscle movement. While the exact cause of ALS is unknown, gain-of-function mutations in SOD1 account for ˜20% of familiar ALS and 2% of spontaneous ALS. In some embodiments, the present disclosure provides compositions of engineered guide RNAs that target SOD1 comprising a barbell macro-footprint sequence and at least some elements of a micro-footprint sequence and facilitate ADAR-mediated RNA editing of SOD1. The engineered guide RNAs of the present disclosure targeting SOD1 and comprising a barbell macro-footprint sequence and at least some elements of a micro-footprint sequence facilitate ADAR-mediated RNA editing of SOD1, thereby, effecting protein knockdown.

In some embodiments, an engineered RNA of the disclosure comprising an engineered guide RNA, which targets an SOD1 start codon, where the engineered guide RNA disclosed here comprises a barbell macro-footprint sequence and at least some elements of a micro-footprint sequence.

In some examples of the disclosure directed to any of the methods of treating a disease or condition in a subject, the subject can be a mammal, for example, a human. The subject of the disclosure can be a subject in need of treatment for a disease or condition, or the subject can be diagnosed with the disease or condition for treatment.

An engineered guide RNA, a polynucleotide encoding the engineered guide RNA of the present disclosure, a delivery vehicle comprising an engineered guide RNA or a polynucleotide encoding the engineered guide RNA of the disclosure, or a pharmaceutical composition comprising any of these can be used in a method of treating a disease or condition or in uses for preparing a medicament or for treating a disease or condition in a subject in need thereof. Some embodiments described here are directed to the use of the engineered polynucleotide or the engineered guide RNA of the disclosure for treating a disease, disorder, or condition in a subject in need thereof. Other embodiments described here provide for a method of treating a disease or condition in a subject in need thereof, where the method comprises administering to the subject, an effective amount of: (a) any of the engineered guide RNAs comprising a barbell macro-footprint sequence and at least some elements of a micro-footprint sequence described here; any of the polynucleotides encoding any of the engineered guide RNAs comprising a barbell macro-footprint sequence and at least some elements of a micro-footprint sequence described here; any of the delivery vehicles comprising: any of the engineered guide RNAs described here or any of the polynucleotides encoding any of the engineered guide RNAs described here; or any of the pharmaceutical compositions comprising: any of the engineered guide RNAs described here, any of the polynucleotides comprising any of the engineered guide RNAs described here, or any of the delivery vehicles comprising any of the engineered guide RNAs described here or any of the polynucleotides encoding any of the engineered guide RNAs described here; and (b) a pharmaceutically acceptable: excipient, diluent, or carrier, where the method treats the disease or condition in the subject. A disorder can be a disease, a condition, a genotype, a phenotype, or any state associated with an adverse effect. In some embodiments, treating a disease, condition, or disorder can comprise preventing, slowing progression of, reversing, or alleviating symptoms of the disease, condition, or disorder. A method of treating a disease, disorder, or condition can comprise in some embodiments, administering or delivering: any of the engineered guide RNAs comprising a barbell macro-footprint sequence and at least some elements of a micro-footprint sequence of the disclosure; any of the engineered polynucleotides encoding for any of the engineered guide RNAs of the disclosure; any of the delivery vehicles comprising any of the engineered guide RNAs comprising a barbell macro-footprint sequence and at least some elements of a micro-footprint sequence or any of the polynucleotides encoding the engineered guide RNAs described here; or any of the pharmaceutical compositions comprising: any of the engineered guide RNAs of the disclosure; any of the engineered polynucleotides encoding for any of the engineered guide RNAs of the disclosure; any of the delivery vehicles comprising any of the engineered guide RNAs disclosed here or any of the polynucleotides encoding the engineered guide RNAs described here, and a pharmaceutically acceptable: excipient, diluent, or carrier, where the method comprises administering or delivering any of the aforementioned to a subject or a cell of a subject in need thereof, to treat the disease, disorder, or condition in the subject. In some examples, the methods of treating a disease or condition.

In some embodiments, a method of treating a disease, disorder, or condition can comprise, administering or delivering any of the engineered polynucleotides encoding for any of the engineered guide RNAs comprising a barbell macro-footprint sequence and at least some elements of a micro-footprint sequence to a subject or a cell of a subject in need thereof expressing the engineered guide RNA in the subject or the cell of the subject in need thereof, to treat the disease, disorder, or condition in the subject. In some embodiments, an engineered guide RNA of the present disclosure can be used to treat a genetic disorder (e.g., FSHD, DM1, CMT1A, or ALS). In some embodiments, an engineered guide RNA comprising a barbell macro-footprint sequence and at least some elements of a micro-footprint sequence of the present disclosure can be used to treat a condition associated with one or more mutations. For example, disclosed herein are methods of treating FSHD with engineered guide RNAs targeting DUX4, where the engineered guide RNAs comprise a barbell macro-footprint sequence and at least some elements of a micro-footprint sequence. Also disclosed herein are methods of treating DM1 with engineered guide RNAs targeting DMPK, where the engineered guide RNAs comprise a barbell macro-footprint sequence and at least some elements of a micro-footprint sequence. Also disclosed herein are methods of treating CMT1A with engineered guide RNAs targeting PMP22, where the engineered guide RNAs comprise a barbell macro-footprint sequence and at least some elements of a micro-footprint sequence. Also disclosed herein are methods of treating ALS with engineered guide RNAs targeting SOD1, where the engineered guide RNAs comprise a barbell macro-footprint sequence and at least some elements of a micro-footprint sequence.

In some embodiments, the target RNA can be selected from the group consisting of: ABCA4, ALAS1, APP, ATP7B, CFTR, DMD, DMPK, DUX4, GAPDH, GBA, HEXA, HFE, LIPA, LRRK2, MAPT, PCSK9 start site, PINK1, PMP22, SERPINA1, SCNN1A start site, SNCA, or SOD1, a fragment of any one of these, and any combination thereof. In some embodiments, the target RNA can be ABCA4. In some embodiments, the ABCA4 can comprise a mutation selected from the group consisting of: G6320A; G5714A; G5882A; and any combination thereof. In some embodiments, the target RNA can be APP. In some embodiments, the engineered RNA can be configured to facilitate an edit of a base of a nucleotide of the APP target RNA by an RNA editing entity, such that a protein translated from the edited target RNA comprises at least one amino acid residue difference as compared to a wildtype APP polypeptide. In some embodiments, the at least one amino acid residue difference can be selected from the group consisting of: K670E, K670R, K670G, M671V, A673V, A673T, D672G, E682G, H684R, K687R, K687E, K687G, I712X, T714X of the APP polypeptide, and any combination thereof. In some embodiments, the target RNA can be SERPINA1. In some embodiments, the SERPINA1 can comprise a mutation of G9989A. In some embodiments, the target RNA can be SERPINA1 encoding a polypeptide with an E342K mutation, relative to a wildtype SERPINA1 polypeptide. In some embodiments, the target RNA can be LRRK2. In some embodiments, the LRRK2 RNA can encode a mutation in a polypeptide encoded by the target RNA, where the mutation can be selected from the group consisting of: E10L, A30P, S52F, E46K, A53T, L119P, A211V, C228S, E334K, N363S, V366M, A419V, R506Q, N544E, N551K, A716V, M712V, 1723V, P755L, R793M, 1810V, K871E, Q923H, Q930R, R1067Q, S1096C, Q1111H, 11122V, A1151T, L1165P, 11192V, H1216R, S1228T, P1262A, R1325Q, I1371V, R1398H, T1410M, D1420N, N1437H, R1441C, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, Q2490N, and any combination thereof. In some embodiments, the target RNA can be SNCA. In some embodiments, the engineered RNA can target a region of the SNCA RNA selected from the group consisting of: a translation initiation site (TIS) AUG to GTG in Codon 1, a TIS AUG in Codon 5, an AUG at position 265 in Exon 2, and any combination thereof.

### Administration

Administration can refer to methods that can be used to enable the delivery of a composition described herein (e.g., an engineered guide RNA) to the desired site of biological action. For example, an engineered guide RNA can be comprised in a DNA construct, a viral vector, or both and be administered by intravenous administration. In some embodiments of the disclosure, the uses or methods of treating as described here, can provide for various routes of administration. Administration disclosed herein to an area in need of treatment or therapy can be achieved by, for example, and not by way of limitation, oral administration, topical administration, intravenous administration, inhalation administration, or any combination thereof. In some embodiments, delivery or administration can include inhalation, otic, buccal, conjunctival, dental, endocervical, endosinusial, endotracheal, enteral, epidural, extra-amniotic, extracorporeal, hemodialysis, infiltration, injection (e.g., parenchymal injection, intra-thecal injection, intra-ventricular injection, intra-cisternal injection, intravenous injection), interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intranasal, intraocular, intraovarian, intraparenchymal, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration, or a combination thereof. Delivery can include direct application to the affected tissue or region of the body. In some cases, topical administration can comprise administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin. Delivery can include a parenchymal injection, an intra-thecal injection, an intra-ventricular injection, an intra-cisternal injection, or an intravenous injection. A composition provided herein can be administered by any delivery method or route of administration. A method of administration can be by intra-arterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump, or a combination thereof. In some embodiments, delivery can be by an enema, an eye drop, a nasal spray, or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). In some embodiments, a medical professional can administer the composition.

In some cases, administering can be oral ingestion. In some cases, delivery can be a capsule or a tablet. Oral ingestion delivery can comprise a tea, an elixir, a food, a drink, a beverage, a syrup, a liquid, a gel, a capsule, a tablet, an oil, a tincture, or any combination thereof. In some embodiments, a food can be a medical food. In some instances, a capsule can comprise hydroxymethylcellulose. In some embodiments, a capsule can comprise a gelatin, hydroxypropylmethyl cellulose, pullulan, or any combination thereof. In some cases, capsules can comprise a coating, for example, an enteric coating. In some embodiments, a capsule can comprise a vegetarian product or a vegan product such as a hypromellose capsule. In some embodiments, delivery can comprise inhalation by an inhaler, a diffuser, a nebulizer, a vaporizer, or a combination thereof.

In some embodiments, disclosed herein can be a method, comprising administering a composition disclosed herein to a subject (e.g., a human) in need thereof. In some instances, the method can treat or prevent a disease in the subject.

### Definitions

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

A double stranded RNA (dsRNA) substrate (e.g., a guide-target RNA scaffold) is formed upon hybridization of an engineered guide RNA of the present disclosure to a target RNA. As disclosed herein, a “bulge” refers to the structure substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can have from 0 to 4 contiguous nucleotides on the guide RNA side of the guide-target RNA scaffold and 1 to 4 contiguous nucleotides on the target RNA side of the guide-target RNA scaffold or a bulge can have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

As described here, a double stranded RNA (dsRNA) substrate (e.g., a guide-target RNA scaffold) is formed upon hybridization of an engineered guide RNA of the present disclosure to a target RNA. A bulge can be a symmetrical bulge or an asymmetrical bulge. A “symmetrical bulge” is formed when the same number of nucleotides is present on each side of the bulge. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold target and 2 nucleotides on the target RNA side of the guide-target RNA scaffold. A symmetrical bulge of the present disclosure can be formed by 3 nucleotides on the engineered guide RNA side of the guide-target RNA scaffold target and 3 nucleotides on the target RNA side of the guide-target RNA scaffold. A symmetrical bulge of the present disclosure can be formed by 4 nucleotides on the engineered guide RNA side of the guide-target RNA scaffold target and 4 nucleotides on the target RNA side of the guide-target RNA scaffold. Thus, a symmetrical bulge can be a structural feature formed from latent structure provided by an engineered latent guide RNA.

As disclosed here, a double stranded RNA (dsRNA) substrate (e.g., a guide-target RNA scaffold) is formed upon hybridization of an engineered guide RNA of the present disclosure to a target RNA. A bulge can be a symmetrical bulge or an asymmetrical bulge. An “asymmetrical bulge” is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. An asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

As disclosed herein, a “base paired (bp) region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold. In some embodiments, a base paired region has from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

“Canonical amino acids” refer to those 20 amino acids that occur in nature, including for example, the amino acids shown in TABLE 1.

TABLE 1
Naturally occurring amino acids indicated with the three letter abbreviations, one letter abbreviations,
structures, and corresponding codons
Non-polar, aliphatic residues

| | | | | |
|---|---|---|---|---|
| Glycine | Gly | G | 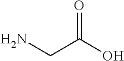 | GGU GGC GGA GGG |
| Alanine | Ala | A | 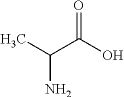 | GCU GCC GCA GCG |
| Valine | Val | V | 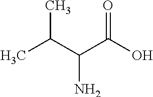 | GUU GUC GUA GUG |
| Leucine | Leu | L | 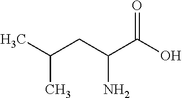 | UUA UUG CUU CUC CUA CUG |
| Isoleucine | Ile | I | 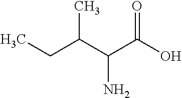 | AUU AUC AUA |
| Proline | Pro | P | 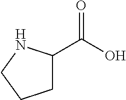 | CCU CCC CCA CCG |

Aromatic residues

| | | | | |
|---|---|---|---|---|
| Phenylalanine | Phe | F | 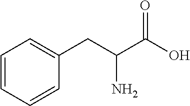 [SMILES: NC(Cc1ccccc1)C(=O)O] | UUU UUC |
| Tyrosine | Tyr | Y | 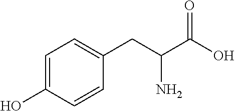 [SMILES: NC(Cc1ccc(O)cc1)C(=O)O] | UAU UAC |
| Tryptophan | Trp | W | 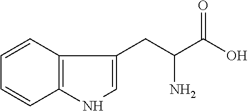 [SMILES: NC(Cc1c[nH]c2ccccc12)C(=O)O] | UGG |

Polar, non-charged residues

| | | | | |
|---|---|---|---|---|
| Serine | Ser | S | 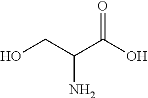 | UCU UCC UCA UCG AGU AGC |
| Threonine | Thr | T | 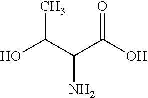 | ACU ACC ACA ACG |
| Cysteine | Cys | C | 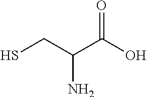 | UGU UGC |
| Methionine | Met | M | 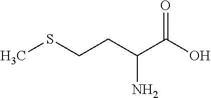 | AUG |
| Asparagine | Asn | N | 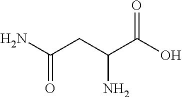 | AAU AAC |
| Glutamine | Gln | Q | 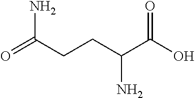 | CAA CAG |

Positively charged residues

| | | | | |
|---|---|---|---|---|
| Lysine | Lys | K | 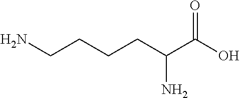 | AAA AAG |
| Arginine | Arg | R | 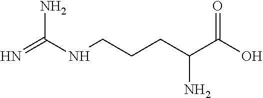 [SMILES: N=C(N)NCCCC(N)C(=O)O] | CGU CGC CGA CGG AGA AGG |
| Histidine | His | H | 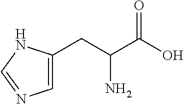 [SMILES: NC(Cc1cnc[nH]1)C(=O)O] | CAU CAC |

Negatively charged residues

| | | | | |
|---|---|---|---|---|
| Aspartate | Asp | D | 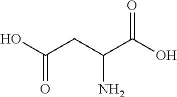 | GAU GAC |
| Glutamate | Glu | E | 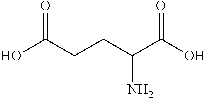 | GAA GAG |

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%, 97%, 98%, 99%, or 100% over a region of 10, 15, 20, 25, 30, 35, 40, 45, 50, or more nucleotides, or can refer to two nucleic acids that hybridize under stringent conditions (e.g., stringent hybridization conditions). Nucleic acids can include nonspecific sequences. As used herein, the term “nonspecific sequence” or “not specific” can refer to a nucleic acid sequence that contains a series of residues that may not be designed to be complementary to or can be only partially complementary to any other nucleic acid sequence.

The terms “determining,” “measuring,” “evaluating,” “assessing,” “assaying,” and “analyzing” can be used interchangeably herein to refer to forms of measurement. The terms include determining if an element can be present or not (for example, detection). These terms can include quantitative, qualitative or quantitative and qualitative determinations. Assessing can be relative or absolute. “Detecting the presence of” can include determining the amount of something present in addition to determining whether it can be present or absent depending on the context.

The term “encode,” as used herein, refers to an ability of a polynucleotide to provide information or instructions sequence sufficient to produce a corresponding gene expression product. In a non-limiting example, mRNA can encode for a polypeptide during translation, whereas DNA can encode for an mRNA molecule during transcription.

As disclosed here, a double stranded RNA (dsRNA) substrate is formed upon hybridization of an engineered guide RNA of the present disclosure to a target RNA. The resulting dsRNA substrate is also referred to herein as a “guide-target RNA scaffold.” Described herein are “structural features” that can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

A “guide-target RNA scaffold,” as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more features selected from the group consisting of a bulge, mismatch, internal loop, hairpin, wobble base pair, and any combination thereof.

As disclosed herein, a “hairpin” is an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can include a recruitment hairpin or a non-recruitment hairpin. A hairpin can be located anywhere within the engineered guide RNAs of the present disclosure. In some embodiments, one or more hairpins is proximal to or present at the 3′ end of an engineered guide RNA of the present disclosure, proximal to or at the 5′ end of an engineered guide RNA of the present disclosure, proximal to or within the targeting domain of the engineered guide RNAs of the present disclosure, or any combination thereof.

As disclosed herein, a hairpin can refer to a recruitment hairpin, a non-recruitment hairpin, or any combination thereof. A “recruitment hairpin,” as disclosed herein, can recruit at least in part an RNA editing entity, such as ADAR. In some cases, a recruitment hairpin can be formed and present in the absence of binding to a target RNA. In some embodiments, a recruitment hairpin is a GluR2 domain or portion thereof. In some embodiments, a recruitment hairpin is an Alu domain or portion thereof. A recruitment hairpin, as defined herein, can include a naturally occurring ADAR substrate or truncations thereof. Thus, a recruitment hairpin such as GluR2 is a pre-formed structural feature that can be present in constructs comprising an engineered guide RNA, not a structural feature formed by latent structure provided in an engineered latent guide RNA. A recruitment hairpin, as described herein, can be a naturally occurring ADAR substrate or truncations thereof.

A “non-recruitment hairpin,” as disclosed herein, does not have a primary function of recruiting an RNA editing entity. A non-recruitment hairpin, in some instances, does not recruit an RNA editing entity. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by latent structure provided in an engineered latent guide RNA.

As used herein, the term percent “identity,” in the context of two or more nucleic acid or polypeptide sequences, can refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity can be performed using the BLAST algorithm, which is described in Altschul et al. (J. Mol. Biol. 215:403-410 (1990)) and incorporated herein by reference for its teachings in its entirety. Software for performing BLAST analyses is publicly available through the National Center for Biotechnology Information.

In some embodiments, a double stranded RNA (dsRNA) substrate (e.g., a guide-target RNA scaffold) is formed upon hybridization of an engineered guide RNA of the present disclosure to a target RNA. As disclosed herein, an “internal loop” refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can help with base flipping of the target A in the target RNA to be edited.

One side of the internal loop, either on the target RNA side or the engineered polynucleotide side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1000 nucleotides. Thus, an internal loop can be a structural feature formed from latent structure provided by an engineered latent guide RNA.

As described here, a double stranded RNA (dsRNA) substrate (e.g., a guide-target RNA scaffold) is formed upon hybridization of an engineered guide RNA of the present disclosure to a target RNA. An internal loop can be a symmetrical internal loop or an asymmetrical internal loop. A “symmetrical internal loop” is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 15 nucleotides on the engineered polynucleotide side of the guide-target RNA scaffold target and 15 nucleotides on the target RNA side of the guide-target RNA scaffold. A symmetrical internal loop of the present disclosure can be formed by 20 nucleotides on the engineered polynucleotide side of the guide-target RNA scaffold target and 20 nucleotides on the target RNA side of the guide-target RNA scaffold. A symmetrical internal loop of the present disclosure can be formed by 30 nucleotides on the engineered polynucleotide side of the guide-target RNA scaffold target and 30 nucleotides on the target RNA side of the guide-target RNA scaffold. A symmetrical internal loop of the present disclosure can be formed by 40 nucleotides on the engineered polynucleotide side of the guide-target RNA scaffold target and 40 nucleotides on the target RNA side of the guide-target RNA scaffold. A symmetrical internal loop of the present disclosure can be formed by 50 nucleotides on the engineered polynucleotide side of the guide-target RNA scaffold target and 50 nucleotides on the target RNA side of the guide-target RNA scaffold. A symmetrical internal loop of the present disclosure can be formed by 60 nucleotides on the engineered polynucleotide side of the guide-target RNA scaffold target and 60 nucleotides on the target RNA side of the guide-target RNA scaffold. A symmetrical internal loop of the present disclosure can be formed by 70 nucleotides on the engineered polynucleotide side of the guide-target RNA scaffold target and 70 nucleotides on the target RNA side of the guide-target RNA scaffold. A symmetrical internal loop of the present disclosure can be formed by 80 nucleotides on the engineered polynucleotide side of the guide-target RNA scaffold target and 80 nucleotides on the target RNA side of the guide-target RNA scaffold. A symmetrical internal loop of the present disclosure can be formed by 90 nucleotides on the engineered polynucleotide side of the guide-target RNA scaffold target and 90 nucleotides on the target RNA side of the guide-target RNA scaffold. A symmetrical internal loop of the present disclosure can be formed by 100 nucleotides on the engineered polynucleotide side of the guide-target RNA scaffold target and 100 nucleotides on the target RNA side of the guide-target RNA scaffold. A symmetrical internal loop of the present disclosure can be formed by 110 nucleotides on the engineered polynucleotide side of the guide-target RNA scaffold target and 110 nucleotides on the target RNA side of the guide-target RNA scaffold. A symmetrical internal loop of the present disclosure can be formed by 120 nucleotides on the engineered polynucleotide side of the guide-target RNA scaffold target and 120 nucleotides on the target RNA side of the guide-target RNA scaffold. A symmetrical internal loop of the present disclosure can be formed by 130 nucleotides on the engineered polynucleotide side of the guide-target RNA scaffold target and 130 nucleotides on the target RNA side of the guide-target RNA scaffold. A symmetrical internal loop of the present disclosure can be formed by 140 nucleotides on the engineered polynucleotide side of the guide-target RNA scaffold target and 140 nucleotides on the target RNA side of the guide-target RNA scaffold. A symmetrical internal loop of the present disclosure can be formed by 150 nucleotides on the engineered polynucleotide side of the guide-target RNA scaffold target and 150 nucleotides on the target RNA side of the guide-target RNA scaffold. A symmetrical internal loop of the present disclosure can be formed by 200 nucleotides on the engineered polynucleotide side of the guide-target RNA scaffold target and 200 nucleotides on the target RNA side of the guide-target RNA scaffold. A symmetrical internal loop of the present disclosure can be formed by 250 nucleotides on the engineered polynucleotide side of the guide-target RNA scaffold target and 250 nucleotides on the target RNA side of the guide-target RNA scaffold. A symmetrical internal loop of the present disclosure can be formed by 300 nucleotides on the engineered polynucleotide side of the guide-target RNA scaffold target and 300 nucleotides on the target RNA side of the guide-target RNA scaffold. A symmetrical internal loop of the present disclosure can be formed by 350 nucleotides on the engineered polynucleotide side of the guide-target RNA scaffold target and 350 nucleotides on the target RNA side of the guide-target RNA scaffold. A symmetrical internal loop of the present disclosure can be formed by 400 nucleotides on the engineered polynucleotide side of the guide-target RNA scaffold target and 400 nucleotides on the target RNA side of the guide-target RNA scaffold. A symmetrical internal loop of the present disclosure can be formed by 450 nucleotides on the engineered polynucleotide side of the guide-target RNA scaffold target and 450 nucleotides on the target RNA side of the guide-target RNA scaffold. A symmetrical internal loop of the present disclosure can be formed by 500 nucleotides on the engineered polynucleotide side of the guide-target RNA scaffold target and 500 nucleotides on the target RNA side of the guide-target RNA scaffold. A symmetrical internal loop of the present disclosure can be formed by 600 nucleotides on the engineered polynucleotide side of the guide-target RNA scaffold target and 600 nucleotides on the target RNA side of the guide-target RNA scaffold. A symmetrical internal loop of the present disclosure can be formed by 700 nucleotides on the engineered polynucleotide side of the guide-target RNA scaffold target and 700 nucleotides on the target RNA side of the guide-target RNA scaffold. A symmetrical internal loop of the present disclosure can be formed by 800 nucleotides on the engineered polynucleotide side of the guide-target RNA scaffold target and 800 nucleotides on the target RNA side of the guide-target RNA scaffold. A symmetrical internal loop of the present disclosure can be formed by 900 nucleotides on the engineered polynucleotide side of the guide-target RNA scaffold target and 900 nucleotides on the target RNA side of the guide-target RNA scaffold. A symmetrical internal loop of the present disclosure can be formed by 1000 nucleotides on the engineered polynucleotide side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

As disclosed here, a double stranded RNA (dsRNA) substrate (e.g., a guide-target RNA scaffold) is formed upon hybridization of an engineered guide RNA of the present disclosure to a target RNA. An internal loop can be a symmetrical internal loop or an asymmetrical internal loop. An “asymmetrical internal loop” is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered polynucleotide side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered polynucleotide side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered polynucleotide side of the guide-target RNA scaffold. An asymmetrical internal loop of the present disclosure can be formed by 5 nucleotides on the target RNA side of the guide-target RNA scaffold and 100 nucleotides on the engineered polynucleotide side of the guide-target RNA scaffold. An asymmetrical internal loop of the present disclosure can be formed by 5 nucleotides on the target RNA side of the guide-target RNA scaffold and 150 nucleotides on the engineered polynucleotide side of the guide-target RNA scaffold. An asymmetrical internal loop of the present disclosure can be formed by 5 nucleotides on the target RNA side of the guide-target RNA scaffold and 200 nucleotides on the engineered polynucleotide side of the guide-target RNA scaffold. An asymmetrical internal loop of the present disclosure can be formed by 5 nucleotides on the target RNA side of the guide-target RNA scaffold and 300 nucleotides on the engineered polynucleotide side of the guide-target RNA scaffold. An asymmetrical internal loop of the present disclosure can be formed by 5 nucleotides on the target RNA side of the guide-target RNA scaffold and 400 nucleotides on the engineered polynucleotide side of the guide-target RNA scaffold. An asymmetrical internal loop of the present disclosure can be formed by 5 nucleotides on the target RNA side of the guide-target RNA scaffold and 500 nucleotides on the engineered polynucleotide side of the guide-target RNA scaffold. An asymmetrical internal loop of the present disclosure can be formed by 5 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 5 nucleotides on the engineered polynucleotide side of the guide-target RNA scaffold. An asymmetrical internal loop of the present disclosure can be formed by 500 nucleotides on the target RNA side of the guide-target RNA scaffold and 5 nucleotides on the engineered polynucleotide side of the guide-target RNA scaffold. An asymmetrical internal loop of the present disclosure can be formed by 400 nucleotides on the target RNA side of the guide-target RNA scaffold and 5 nucleotides on the engineered polynucleotide side of the guide-target RNA scaffold. An asymmetrical internal loop of the present disclosure can be formed by 300 nucleotides on the target RNA side of the guide-target RNA scaffold and 5 nucleotides on the engineered polynucleotide side of the guide-target RNA scaffold. An asymmetrical internal loop of the present disclosure can be formed by 200 nucleotides on the target RNA side of the guide-target RNA scaffold and 5 nucleotides on the engineered polynucleotide side of the guide-target RNA scaffold. An asymmetrical internal loop of the present disclosure can be formed by 150 nucleotides on the target RNA side of the guide-target RNA scaffold and 5 nucleotides on the engineered polynucleotide side of the guide-target RNA scaffold. An asymmetrical internal loop of the present disclosure can be formed by 100 nucleotides on the target RNA side of the guide-target RNA scaffold and 5 nucleotides on the engineered polynucleotide side of the guide-target RNA scaffold. An asymmetrical internal loop of the present disclosure can be formed by 50 nucleotides on the target RNA side of the guide-target RNA scaffold and 5 nucleotides on the engineered polynucleotide side of the guide-target RNA scaffold. An asymmetrical internal loop of the present disclosure can be formed by 50 nucleotides on the target RNA side of the guide-target RNA scaffold and 100 nucleotides on the engineered polynucleotide side of the guide-target RNA scaffold. An asymmetrical internal loop of the present disclosure can be formed by 50 nucleotides on the target RNA side of the guide-target RNA scaffold and 150 nucleotides on the engineered polynucleotide side of the guide-target RNA scaffold. An asymmetrical internal loop of the present disclosure can be formed by 50 nucleotides on the target RNA side of the guide-target RNA scaffold and 200 nucleotides on the engineered polynucleotide side of the guide-target RNA scaffold. An asymmetrical internal loop of the present disclosure can be formed by 50 nucleotides on the target RNA side of the guide-target RNA scaffold and 300 nucleotides on the engineered polynucleotide side of the guide-target RNA scaffold. An asymmetrical internal loop of the present disclosure can be formed by 50 nucleotides on the target RNA side of the guide-target RNA scaffold and 400 nucleotides on the engineered polynucleotide side of the guide-target RNA scaffold. An asymmetrical internal loop of the present disclosure can be formed by 50 nucleotides on the target RNA side of the guide-target RNA scaffold and 500 nucleotides on the engineered polynucleotide side of the guide-target RNA scaffold. An asymmetrical internal loop of the present disclosure can be formed by 50 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 50 nucleotides on the engineered polynucleotide side of the guide-target RNA scaffold. An asymmetrical internal loop of the present disclosure can be formed by 500 nucleotides on the target RNA side of the guide-target RNA scaffold and 50 nucleotides on the engineered polynucleotide side of the guide-target RNA scaffold. An asymmetrical internal loop of the present disclosure can be formed by 400 nucleotides on the target RNA side of the guide-target RNA scaffold and 50 nucleotides on the engineered polynucleotide side of the guide-target RNA scaffold. An asymmetrical internal loop of the present disclosure can be formed by 300 nucleotides on the target RNA side of the guide-target RNA scaffold and 50 nucleotides on the engineered polynucleotide side of the guide-target RNA scaffold. An asymmetrical internal loop of the present disclosure can be formed by 200 nucleotides on the target RNA side of the guide-target RNA scaffold and 50 nucleotides on the engineered polynucleotide side of the guide-target RNA scaffold. An asymmetrical internal loop of the present disclosure can be formed by 150 nucleotides on the target RNA side of the guide-target RNA scaffold and 50 nucleotides on the engineered polynucleotide side of the guide-target RNA scaffold. An asymmetrical internal loop of the present disclosure can be formed by 100 nucleotides on the target RNA side of the guide-target RNA scaffold and 50 nucleotides on the engineered polynucleotide side of the guide-target RNA scaffold. An asymmetrical internal loop of the present disclosure can be formed by 100 nucleotides on the target RNA side of the guide-target RNA scaffold and 150 nucleotides on the engineered polynucleotide side of the guide-target RNA scaffold. An asymmetrical internal loop of the present disclosure can be formed by 100 nucleotides on the target RNA side of the guide-target RNA scaffold and 200 nucleotides on the engineered polynucleotide side of the guide-target RNA scaffold. An asymmetrical internal loop of the present disclosure can be formed by 100 nucleotides on the target RNA side of the guide-target RNA scaffold and 300 nucleotides on the engineered polynucleotide side of the guide-target RNA scaffold. An asymmetrical internal loop of the present disclosure can be formed by 100 nucleotides on the target RNA side of the guide-target RNA scaffold and 400 nucleotides on the engineered polynucleotide side of the guide-target RNA scaffold. An asymmetrical internal loop of the present disclosure can be formed by 100 nucleotides on the target RNA side of the guide-target RNA scaffold and 500 nucleotides on the engineered polynucleotide side of the guide-target RNA scaffold. An asymmetrical internal loop of the present disclosure can be formed by 100 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 100 nucleotides on the engineered polynucleotide side of the guide-target RNA scaffold. An asymmetrical internal loop of the present disclosure can be formed by 500 nucleotides on the target RNA side of the guide-target RNA scaffold and 100 nucleotides on the engineered polynucleotide side of the guide-target RNA scaffold. An asymmetrical internal loop of the present disclosure can be formed by 400 nucleotides on the target RNA side of the guide-target RNA scaffold and 100 nucleotides on the engineered polynucleotide side of the guide-target RNA scaffold. An asymmetrical internal loop of the present disclosure can be formed by 300 nucleotides on the target RNA side of the guide-target RNA scaffold and 100 nucleotides on the engineered polynucleotide side of the guide-target RNA scaffold. An asymmetrical internal loop of the present disclosure can be formed by 200 nucleotides on the target RNA side of the guide-target RNA scaffold and 100 nucleotides on the engineered polynucleotide side of the guide-target RNA scaffold. An asymmetrical internal loop of the present disclosure can be formed by 150 nucleotides on the target RNA side of the guide-target RNA scaffold and 100 nucleotides on the engineered polynucleotide side of the guide-target RNA scaffold. An asymmetrical internal loop of the present disclosure can be formed by 150 nucleotides on the target RNA side of the guide-target RNA scaffold and 200 nucleotides on the engineered polynucleotide side of the guide-target RNA scaffold. An asymmetrical internal loop of the present disclosure can be formed by 150 nucleotides on the target RNA side of the guide-target RNA scaffold and 300 nucleotides on the engineered polynucleotide side of the guide-target RNA scaffold. An asymmetrical internal loop of the present disclosure can be formed by 150 nucleotides on the target RNA side of the guide-target RNA scaffold and 400 nucleotides on the engineered polynucleotide side of the guide-target RNA scaffold. An asymmetrical internal loop of the present disclosure can be formed by 150 nucleotides on the target RNA side of the guide-target RNA scaffold and 500 nucleotides on the engineered polynucleotide side of the guide-target RNA scaffold. An asymmetrical internal loop of the present disclosure can be formed by 150 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 150 nucleotides on the engineered polynucleotide side of the guide-target RNA scaffold. An asymmetrical internal loop of the present disclosure can be formed by 500 nucleotides on the target RNA side of the guide-target RNA scaffold and 5 nucleotides on the engineered polynucleotide side of the guide-target RNA scaffold. An asymmetrical internal loop of the present disclosure can be formed by 400 nucleotides on the target RNA side of the guide-target RNA scaffold and 150 nucleotides on the engineered polynucleotide side of the guide-target RNA scaffold. An asymmetrical internal loop of the present disclosure can be formed by 300 nucleotides on the target RNA side of the guide-target RNA scaffold and 150 nucleotides on the engineered polynucleotide side of the guide-target RNA scaffold. An asymmetrical internal loop of the present disclosure can be formed by 200 nucleotides on the target RNA side of the guide-target RNA scaffold and 300 nucleotides on the engineered polynucleotide side of the guide-target RNA scaffold. An asymmetrical internal loop of the present disclosure can be formed by 200 nucleotides on the target RNA side of the guide-target RNA scaffold and 400 nucleotides on the engineered polynucleotide side of the guide-target RNA scaffold. An asymmetrical internal loop of the present disclosure can be formed by 200 nucleotides on the target RNA side of the guide-target RNA scaffold and 500 nucleotides on the engineered polynucleotide side of the guide-target RNA scaffold. An asymmetrical internal loop of the present disclosure can be formed by 200 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 200 nucleotides on the engineered polynucleotide side of the guide-target RNA scaffold. An asymmetrical internal loop of the present disclosure can be formed by 500 nucleotides on the target RNA side of the guide-target RNA scaffold and 200 nucleotides on the engineered polynucleotide side of the guide-target RNA scaffold. An asymmetrical internal loop of the present disclosure can be formed by 400 nucleotides on the target RNA side of the guide-target RNA scaffold and 200 nucleotides on the engineered polynucleotide side of the guide-target RNA scaffold. An asymmetrical internal loop of the present disclosure can be formed by 300 nucleotides on the target RNA side of the guide-target RNA scaffold and 200 nucleotides on the engineered polynucleotide side of the guide-target RNA scaffold. An asymmetrical internal loop of the present disclosure can be formed by 300 nucleotides on the target RNA side of the guide-target RNA scaffold and 400 nucleotides on the engineered polynucleotide side of the guide-target RNA scaffold. An asymmetrical internal loop of the present disclosure can be formed by 300 nucleotides on the target RNA side of the guide-target RNA scaffold and 500 nucleotides on the engineered polynucleotide side of the guide-target RNA scaffold. An asymmetrical internal loop of the present disclosure can be formed by 300 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 300 nucleotides on the engineered polynucleotide side of the guide-target RNA scaffold. An asymmetrical internal loop of the present disclosure can be formed by 500 nucleotides on the target RNA side of the guide-target RNA scaffold and 300 nucleotides on the engineered polynucleotide side of the guide-target RNA scaffold. An asymmetrical internal loop of the present disclosure can be formed by 400 nucleotides on the target RNA side of the guide-target RNA scaffold and 300 nucleotides on the engineered polynucleotide side of the guide-target RNA scaffold. An asymmetrical internal loop of the present disclosure can be formed by 400 nucleotides on the target RNA side of the guide-target RNA scaffold and 500 nucleotides on the engineered polynucleotide side of the guide-target RNA scaffold. An asymmetrical internal loop of the present disclosure can be formed by 400 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 400 nucleotides on the engineered polynucleotide side of the guide-target RNA scaffold. An asymmetrical internal loop of the present disclosure can be formed by 500 nucleotides on the target RNA side of the guide-target RNA scaffold and 400 nucleotides on the engineered polynucleotide side of the guide-target RNA scaffold. An asymmetrical internal loop of the present disclosure can be formed by 500 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 500 nucleotides on the engineered polynucleotide side of the guide-target RNA scaffold. Thus, an asymmetrical internal loop can be a structural feature formed from latent structure provided by an engineered latent guide RNA.

“Latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

An “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

“Messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

A double stranded RNA (dsRNA) substrate (e.g., a guide-target RNA scaffold) is formed upon hybridization of an engineered guide RNA of the present disclosure to a target RNA. As disclosed herein, the term “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA. In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA.

The term “mutation” as used herein, can refer to an alteration to a nucleic acid sequence or a polypeptide sequence that can be relative to a reference sequence. A mutation can occur in a DNA molecule, a RNA molecule (e.g., tRNA, mRNA), or in a polypeptide or protein, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. Non-limiting examples of mutations in a nucleic acid sequence that, without the mutation, encodes for a polypeptide sequence, include: “missense” mutations that can result in the substitution of one codon for another, “nonsense” mutations that can change a codon from one encoding a particular amino acid to a stop codon (which can result in truncated translation of proteins), or “silent” mutations that can be those which have no effect on the resulting protein. The mutation can be a “point mutation,” which can refer to a mutation affecting only one nucleotide in a DNA or RNA sequence. The mutation can be a “splice site mutations,” which can be present pre-mRNA (prior to processing to remove introns) resulting in mistranslation and often truncation of proteins from incorrect delineation of the splice site. A mutation can comprise a single nucleotide variation (SNV). A mutation can comprise a sequence variant, a sequence variation, a sequence alteration, or an allelic variant. A mutation can affect function. A mutation may not affect function. A mutation can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. A mutation can be a point mutation. A mutation can comprise a sequence variant, a sequence variation, a sequence alteration, or an allelic variant. The mutation can be a fusion gene. A fusion pair or a fusion gene can result from a mutation, such as a translocation, an interstitial deletion, a chromosomal inversion, or any combination thereof. A mutation can constitute variability in the number of repeated sequences, such as triplications, quadruplications, or others. For example, a mutation can be an increase or a decrease in a copy number associated with a given sequence (e.g., copy number variation, or CNV). A mutation can include two or more sequence changes in different alleles or two or more sequence changes in one allele. A mutation can include two different nucleotides at one position in one allele, such as a mosaic. A mutation can include two different nucleotides at one position in one allele, such as a chimeric. A mutation can be present in a malignant tissue. A presence or an absence of a mutation can indicate an increased risk to develop a disease or condition. A presence or an absence of a mutation can indicate a presence of a disease or condition. A mutation can be present in a benign tissue. Absence of a mutation can indicate that a tissue or sample is benign. As an alternative, absence of a mutation may not indicate that a tissue or sample is benign. Methods as described herein can comprise identifying a presence of a mutation in a sample.

A presence or an absence of a mutation can indicate an increased risk to develop a disease or condition. A presence or an absence of a mutation can indicate a presence of a disease or condition. A mutation can be present in a benign tissue. Absence of a mutation can indicate that a tissue or sample can be benign. As an alternative, absence of a mutation may not indicate that a tissue or sample can be benign. Methods as described herein can comprise identifying a presence of a mutation in a sample.

The terms “polynucleotide” and “oligonucleotide” can be used interchangeably and can refer to a polymeric form of nucleotides of any length, either deoxyribonucleotides or ribonucleotides or analogs thereof. Polynucleotides can have any three-dimensional structure and can perform any function, known or unknown. The following may be non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term can also refer to both double- and single-stranded molecules. Unless otherwise specified or required, any embodiment of this disclosure that can be a polynucleotide encompasses both the double-stranded form and each of two complementary single-stranded forms known or predicted to make up the double-stranded form.

A polynucleotide can be composed of a specific sequence of nucleotides. A nucleotide comprises a nucleoside and a phosphate group. A nucleotide comprises a sugar (e.g., ribose or 2′deoxyribose) and a nucleobase, such as a nitrogenous base. Non-limiting examples of nucleobases include adenine (A), cytosine (C), guanine (G), thymine (T), uracil (U), and inosine (I). In some embodiments, I can be formed when hypoxanthine can be attached to ribofuranose via a P-N9-glycosidic bond, resulting in the chemical structure:

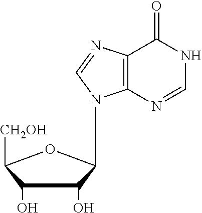

Some polynucleotide embodiments refer to a DNA sequence. In some embodiments, the DNA sequence can be interchangeable with a similar RNA sequence. Some embodiments refer to an RNA sequence. In some embodiments, the RNA sequence can be interchangeable with a similar DNA sequence. In some embodiments, Us and Ts can be interchanged in a sequence provided herein.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together—optionally to preserve the conformation of the fused protein domains, prevent unfavorable interactions between the fused protein domains which can compromise their respective functions, or both.

The term “stop codon” can refer to a three nucleotide contiguous sequence within messenger RNA that signals a termination of translation. Non-limiting examples include in RNA, UAG (amber), UAA (ochre), UGA (umber, also known as opal) and in DNA TAG, TAA or TGA. Unless otherwise noted, the term can also include nonsense mutations within DNA or RNA that introduce a premature stop codon, causing any resulting protein to be abnormally shortened.

The term “structured motif,” as disclosed herein, comprises two or more features in a guide-target RNA scaffold.

The terms “subject,” “individual,” or “patient” can be used interchangeably herein. A “subject” refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some cases, the subject may be not necessarily diagnosed or suspected of being at high risk for the disease

The term “in vivo” refers to an event that takes place in a subject's body.

The term “ex vivo” refers to an event that takes place outside of a subject's body. An ex vivo assay may be not performed on a subject. Rather, it can be performed upon a sample separate from a subject. An example of an ex vivo assay performed on a sample can be an “in vitro” assay.

The term “in vitro” refers to an event that takes places contained in a container for holding laboratory reagent such that it can be separated from the biological source from which the material can be obtained. In vitro assays can encompass cell-based assays in which living or dead cells can be employed. In vitro assays can also encompass a cell-free assay in which no intact cells can be employed.

The term “wobble base pair” refers to two bases that weakly base pair. For example, a wobble base pair of the present disclosure can refer to a G paired with a U. Thus, a wobble base pair can be a structural feature formed from latent structure provided by an engineered latent guide RNA.

As used herein, the terms “treatment” or “treating” can be used in reference to a pharmaceutical or other intervention regimen for obtaining beneficial or desired results in the recipient. Beneficial or desired results include but may not be limited to a therapeutic benefit, a prophylactic benefit, or both. A therapeutic benefit can refer to eradication or amelioration of symptoms or of an underlying disorder being treated. Also, a therapeutic benefit can be achieved with the eradication or amelioration of one or more of the physiological symptoms associated with the underlying disorder such that an improvement can be observed in the subject, notwithstanding that the subject can still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

The following examples are included for illustrative purposes only and are not intended to limit the scope of the present disclosure.

### EXAMPLES

### Example 1

### Exemplary Guide RNA Design Principles

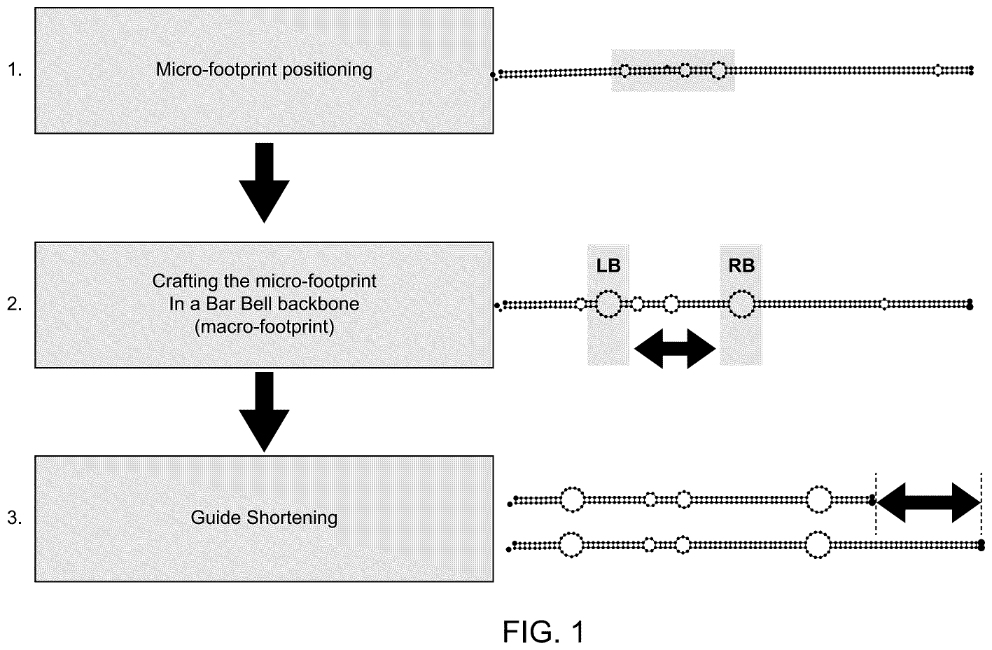

FIG. 1 illustrates exemplary principles for generating engineered guide RNAs having barbell macro-footprint structures as described herein.
(1) Guide RNAs are selected in a high-throughput screen for their ability to facilitate editing of an adenosine of a target RNA via an adenosine deaminase. The selected guide RNAs contain micro-footprint, where the micro-footprint includes a region with latent structures near the 5′ end of the guide-target RNA scaffold and a complementary region near the 3′ end of the guide-target RNA scaffold. The composition of the latent structures and their positioning relative to the mismatch are engineered to generate a superior micro-footprint.
(2) Guide RNAs displaying the ability to facilitate editing are selected. To these guides are added macro-footprint sequences that produce a first internal loop/left barbell (LB) 5′ of the micro-footprint and a second internal loop/right barbell (RB) 3′ of the micro-footprint. The size of each barbell, as well as the positioning of the barbell relative to the micro-footprint, are generated to select guide RNAs with increased editing for an on-target adenosine of interest.
(3) Guide RNAs with the superior barbell of (2) are shortened in length at the 3′ end of the guide-target RNA scaffold as pictured in FIG. 1 or shortened in length at the 5′ end of the guide-target RNA scaffold to generate a superior guide RNA with a micro-footprint and macro-footprint.

Exemplary guide RNAs of the present disclosure are found in TABLE 17.

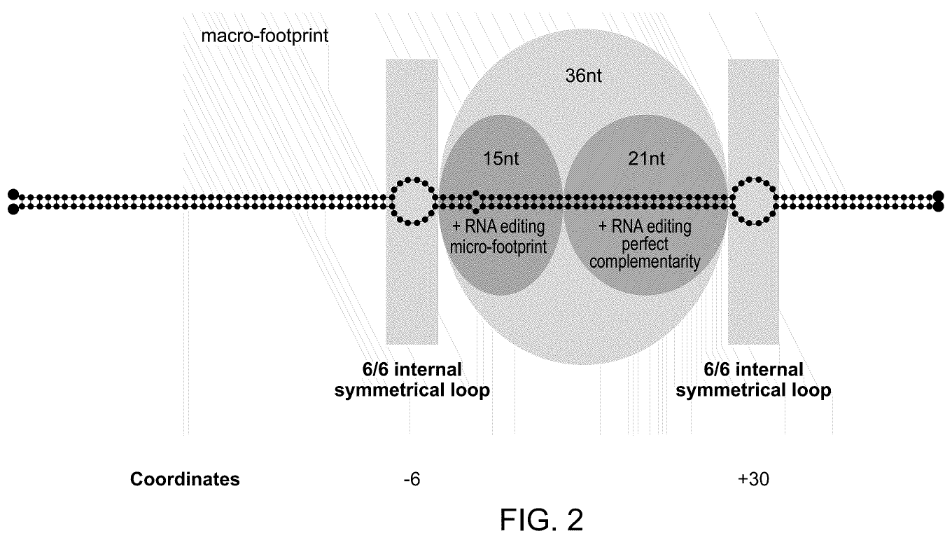

FIG. 2 illustrates a detailed overview of target RNA bound to a guide RNA with micro-footprint latent structures and macro-footprint barbell latent structures that manifest through hybridization with the target RNA.

The guide RNA of FIG. 2 comprises a 36 nt micro-footprint sequence having two regions. First, the exemplary guide has a 15 nt “RNA editing micro-footprint” that comprises a cytosine as the mismatched nucleotide opposite the adenosine to be edited. Second, the exemplary guide has a 21 nt “RNA editing perfect complementarity” sequence with complementarity to the target RNA.

Flanking the 36 nt micro-footprint is a right barbell and left barbell. The exemplary guide illustrated in FIG. 2 provides exemplary barbells that are each symmetrical internal loops, where the symmetrical loops each comprise 6 nucleotides of the guide RNA and 6 nucleotides of the target RNA when the latent structure manifests upon hybridization.

### Example 2

Exemplary Guide RNAs with ADAR1 Editing of ABCA4 G2237A Mutation

Exemplary guide RNAs were screened for facilitating editing of an G2237A point mutation in an ABCA4 mRNA. Each guide contains a micro-footprint sequence that produces micro-footprint latent structures as described herein. Parameters such as the length of the guide and the position of the mismatch were engineered to improve the amount of editing.

The upper panels of FIG. 3 illustrated the RNA editing percentages for controls (no transfection, GFP) and engineered guide RNAs having a total length of 40 bases and greater (e.g., 45, 50, 55, 60, 65, 70, 75, 80, 85, 90, 95, 100) and the upstream length or distance from the target position of ABCA4 (G2237A) (where the ABCA4 RNA comprises a substitution of a G with an A at nucleotide position 2237) was 20 bases or greater (e.g., 25, 30, 35, 40, 45, 50, 55, 60, 65, 70, 75, 80). The RNA editing percentages of ABCA4 engineered RNA guides and controls via ADAR were about 2% to about 16%.

The lower panels of FIG. 3 illustrate ADAR-mediated RNA editing percentages for three biological replicates (center, right) for various guide RNAs. Of the variants screened, the 0.85.65 demonstrated the best editing efficiencies via ADAR, with an editing efficient over 12% at target 0 position (or on-target editing) and minimal off-target edits of about 3%. The dashed line boxed around off-target peaks at target positions +13 and +15 for all three exemplified guides (e.g., 0.100.80 (left); biological replicates: 0.85.65 (center); and 0.85.65 (right)) clearly demonstrate that the 0.85.65 guides had improved editing efficiency, an increase in the amount or percentage of on-target editing, and a decrease in the amount, percentage, or both the amount and percentage of off-target editing.

The 0.85.65 guide RNA was utilized as a scaffold for generating the barbell macro-footprint in the following examples.

### Example 3

### Modifying the Positioning of the Right Barbell for Enhanced ADAR1 Editing of ABCA4 G5882A Mutation

Macro-footprint sequences were inserted into the 0.85.65 guide of Example 2 to produce a left barbell and a right barbell upon hybridization of the guide RNA with the ABCA4 mRNA. In this example, the positioning of the right barbell was engineered based on the improvement in editing efficiency.

FIG. 4 presents various engineered guide RNAs with barbells grafted onto guide 0.85.65 and their associated RNA editing percentages with ADAR1 of ABCA4 G5882A. The position of the second internal loop (RB) relative to the target position of the mismatch (e.g., target 0) was modulated from a position +17, +20, +24, +27, +30, +33, +36, and +39 bases from the target position. The exemplary guides of FIG. 4 (right panel) show that the left-most base of the second internal loop (RB) was positioned at +39, +36, +33, +30, +27, +24, +20, and +17 from target 0. FIG. 4 (left panel) provides the corresponding RNA editing percentages for each of the engineered guides with and without the barbell design (guide 0.85.65), as well as the controls (no transfection, GFP plasmid) via ADAR. Guides 0.85.65 (−5, +33) (˜33% and ˜29% for biological replicates 1 and 2, respectively) and 0.85.65 (−5, +27) (˜23% and ˜21% for biological replicates 1 and 2, respectively) demonstrated RNA editing percentages greater than that of the comparable 0.85.65 guide without or lacking the first and second internal loops (˜15% and ˜13% for biological replicates 1 and 2, respectively).

### Example 4

Comparison of Exemplary Guide RNAs with and without First and Second Internal Loops

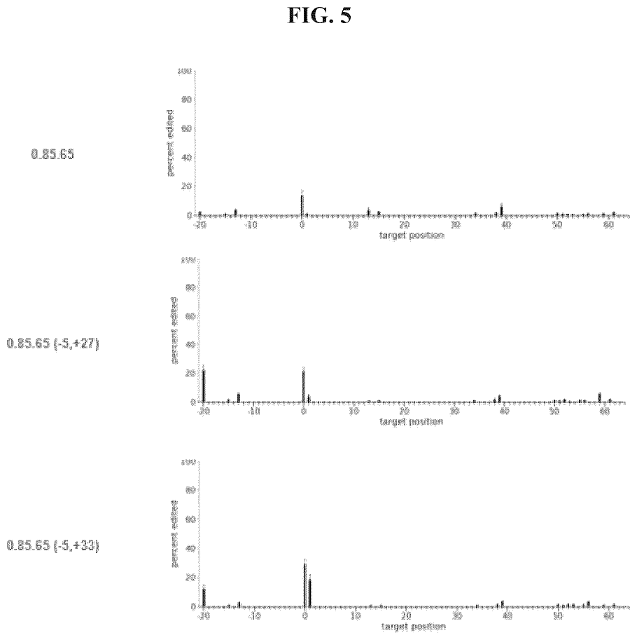

The editing efficiency of engineered guide RNAs provided in Example 3 was determined via ADAR by sequencing to determine the amount of on target and off target editing. FIG. 5 shows the percent edited (Y-axis) at each of the target positions (X-axis), where target 0 represents the position of the intended or desired edit of the ABCA4 G5882A mutation with ADAR1. The top panel demonstrated the RNA editing percentage of less than about 20% at target 0 using guide 0.85.65 without the barbell design. The center panel (guide 0.85.65 (−5, +27)) and bottom panel (guide 0.85.65 (−5, +33) demonstrated a target 0 RNA editing percentage of about 20% or greater and with a decrease in the amount of off-target editing at, for example, positions +13, +15, and +39.

### Example 5

Exemplary Guide RNAs with Varied Coordinates of the Second Internal Loop

The 0.85.65 (−5, +33) guide RNA selected in Example 3 was utilized in this example for selecting superior positioning of the left barbell (LB).

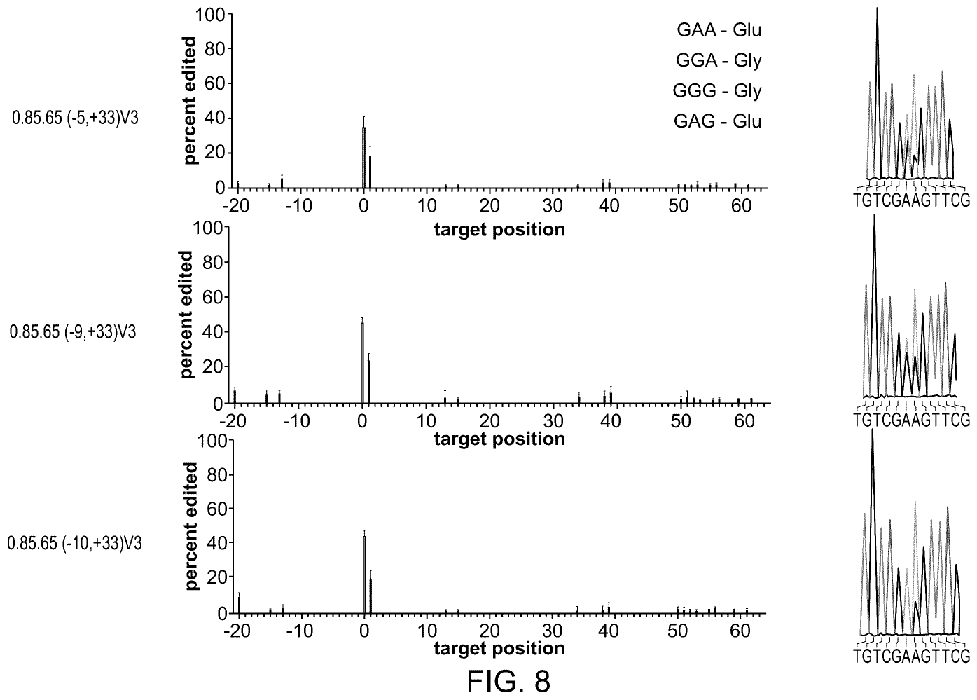

FIG. 6 presents various engineered guide RNAs with barbells grafted onto guide 0.85.65 and their associated RNA editing percentages with ADAR1 of ABCA4 G5882A. The position of the first internal loop (LB) relative to the target position of the mismatch (e.g., target 0) was modulated from a position −5, −6, −7, −8, −9, −10, −11, and −12 from target 0. The exemplary guides of FIG. 7 (right panel) show that the right-most base of the first internal loop (LB) was positioned at −12, −11, −10, −9, −8, −7, −6, and −5 bases from target 0. FIG. 7 (left panel) provides the corresponding RNA editing percentages for each of the engineered guides with and without the barbell design (guide 0.85.65), as well as the controls (no transfection, GFP plasmid). Guides 0.85.65 (−9, +33) and 0.85.65 (−10, +33) (˜50% and ˜46% for biological replicates 1 and 2, respectively) each demonstrated RNA editing percentages via ADAR greater than that of the comparable 0.85.65 guide lacking the first and second internal loops (˜10% and ˜6% for biological replicates 1 and 2, respectively). FIG. 8 illustrates the on-target editing percentages via ADAR for the different guide RNAs 0.85.65 (−5, +33) (top panel); 0.85.65 (−9, +33) (center panel); and 0.85.65 (−10, +33) (bottom panel) as determined by sequencing. The RNA editing percentages of the aforementioned guides via ADAR at target 0, which was the second base of the GAA triplet identified in the top panel, were about 35%, about 45%, and about 45%, respectively. The third base of the GAA triplet was edited at a target position 1, and each of the aforementioned guides had RNA editing percentages of about 20%.

### Example 6

Selecting Mismatch Position for Exemplary Guide RNAs with Improved ADAR1 Editing of ABCA4 G5882A Mutation

The position of the mismatch position was selected for engineered guide RNAs with total guide lengths of 100 nucleotides having improved ADAR1 editing of ABCA4 G5882A mutation.

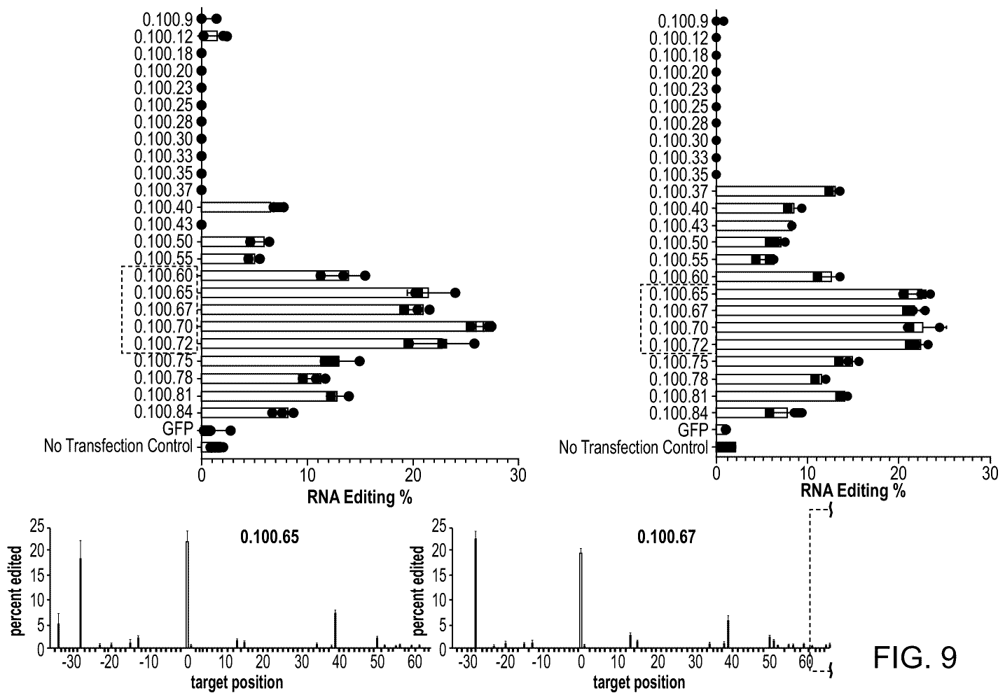

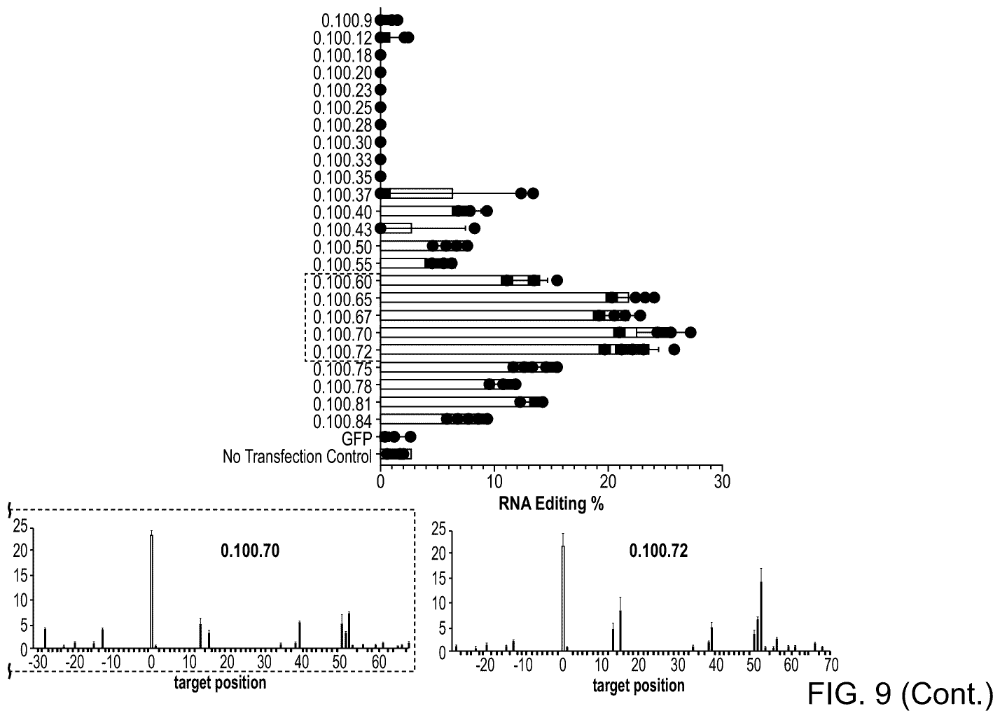

The upper panels of FIG. 9 present various engineered guide RNAs targeting ABCA4 G5882A mutation and controls, and their respective ADAR-mediated RNA editing percentages for two biological replicates (left and center panels) and the average RNA editing percentages (right panel). The position of the mismatch, relative to the guide length, is provided for each guide as the third number in the notation.

Each of RNA guides 0.100.65; 0.100.67; 0.100.70; and 0.100.72 (having the mismatch positioned at position 65, 67, 70, and 72, respectively) had an average ADAR-mediated RNA editing percentage over about 20%. The lower panel provides RNA editing percentages at target positions for the aforementioned RNA guides. Specifically, the ADAR-mediated RNA editing percentages at target 0 for guide RNAs 0.100.65 (˜20%); 0.100.67 (˜20%); 0.100.70 (˜25%); and 0.100.72 (˜23%) were all about 20% or greater.

### Example 7

Exemplary Guide RNAs with ADAR1 Editing of ABCA4 G5882A Mutation with Various Coordinates of the Second Internal Loop

Guide RNA 0.100.70 selected in Example 6 was used to modulate the positions of the second internal loop (RB) (FIG. 12) as in Example 3. The guide RNAs were modified such that the base of the second internal loop most proximal (or nearest) to target 0 position of the guide-target RNA scaffold was distanced at: +12 bases; +14 bases; +16 bases; +18 bases; +20 bases; +23 bases; +25 bases; +27 bases; +30 bases; +32 bases; +34 bases; +36 bases; +38 bases; and +40 bases from the target 0 position, while the base of the first internal loop most proximal (or nearest) to target 0 position of the guide-target RNA scaffold was distanced at −5 bases for all of the guide RNAs.

For example, the exemplary guides of FIG. 10 represented in FIG. 11 (right panel) show that the left-most base of the second internal loop (RB) was positioned at +12 bases; +14 bases; +16 bases; +18 bases; +20 bases; +23 bases; +25 bases; +27 bases; +30 bases; +32 bases; +34 bases; +36 bases; +38 bases; and +40 bases from target 0. FIG. 11 (left panel) provides the corresponding RNA editing percentages for each of the engineered guides via ADAR1 with and without the first and second internal loops (e.g., guide 0.100.70), as well as controls (no transfection, GFP plasmid). Guide 0.100.70 (−5, +32) (˜20% and ˜24% for biological replicates 1 and 2, respectively) demonstrated RNA editing percentages greater than that of the comparable 0.100.70 guide without or lacking the first and second internal loops (˜16% for each of biological replicates 1 and 2).

### Example 8

Exemplary Guide RNAs with ADAR1 Editing of ABCA4 G5882A Mutation with Various Coordinates of the First Internal Loop

Guide RNA 0.100.70 was used to modulate the positions of the first internal loop (LB) (FIG. 12) as in Example 5. The guide RNAs were modified such that the base of the first internal loop most proximal (or nearest) to target 0 position of the guide-target RNA scaffold was distanced at: −15 bases; −14 bases; −13 bases; −12 bases; −11 bases; −9 bases; −6 bases; −5 bases (−5, +33) and (−5, +32) from the target 0 position, while the base of the second internal loop most proximal (or nearest) to target 0 position of the guide-target RNA scaffold was distanced at +33 bases for all of the guide RNAs except guide 0.100.70 (−5, +32) and guide 0.100.70 without the first and second internal loops.

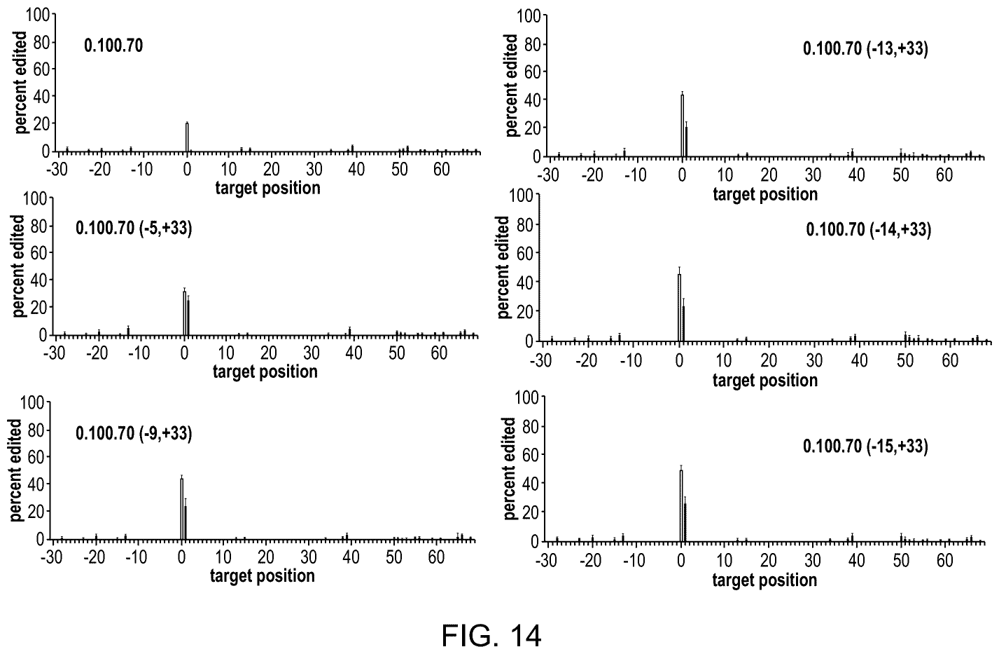

For example, the exemplary guides of FIG. 12 represented in FIG. 13 (right panel) show that the right-most base of the first internal loop (LB) was positioned at −15 bases; −14 bases; −13 bases; −12 bases; −11 bases; −9 bases; −6 bases; and −5 bases from target 0. FIG. 13 (left panel) provides the corresponding ADAR-mediated RNA editing percentages for each of the engineered guides with and without the first and second internal loops (e.g., guide 0.100.70), as well as controls (no transfection, GFP plasmid). All of the exemplary guide RNAs demonstrated ADAR-mediated RNA editing percentages of about 34%, except for guide 0.100.70 (−5, +32) and guide 0.100.70, which had ADAR-mediated RNA editing percentages of about 20% and the controls. FIG. 14 provides ADAR-mediated RNA editing percentages for on-target editing at target 0 for guide RNAs 0.100.70 (−5, +33) (center left panel); 0.100.70 (−9, +33) (bottom left panel); 0.100.70 (−13, +33) (top right panel); 0.100.70 (−14, +33) (center right panel); 0.100.70 (−15, +33) (bottom right panel). The aforementioned guides had ADAR-mediated RNA editing percentages (˜30%; ˜40%; about >40%; ˜45%; ˜45%) greater than the ADAR-mediated RNA editing percentage of ˜20% of the comparable 0.100.70 guide (top left panel) which lacks the first and second internal loops.

### Example 9

Exemplary Guide RNAs with ADAR1 Editing of ABCA4 G5882A Mutation Shortened from Guides 100.70 (−9, +33) and (−15, +33)

The guide RNAs produced in Example 8 with superior right and left barbells (guide 0.100.70 (−9, +33) and guide 0.100.70 (−15, +33)), each based on the 0.100.70 guide RNA, were further engineered to reduce the total guide RNA length for the region 3′ of the left barbell.

In FIG. 15, guide 0.100.70 (−9, +33) and guide 0.100.70 (−15, +33) were shortened such that the length ranged from 78 nucleotides to 100 nucleotides and the 3′ end of the dsRNA from target 0 position ranged from 48 nucleotides to 70 nucleotides, where each nucleotide was composed of a nucleic acid base. Exemplary guide RNAs were 0.78.48; 0.80.50; 0.82.52; 0.84.54; 0.86.56; 0.88.58; 0.90.60; 0.92.62; 0.94.64; 0.96.66; 0.98.68; 0.100.70, where a first internal loop was located at −9 or −15 upstream and +33 downstream of target 0 position. The various guide RNAs of 0.100.70 (−9, +33) were represented in FIG. 16 (right panel).

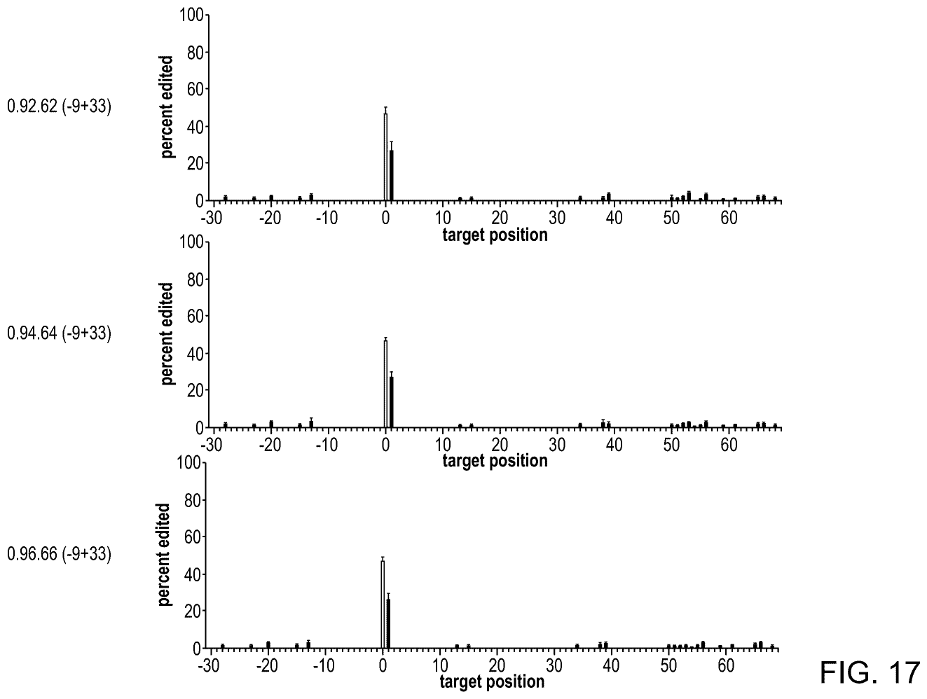

For example, the exemplary guides of FIG. 15 based on guide 0.100.70 (−9, +33) represented in FIG. 16 (right panel) show that the 3′ end of the dsRNA had a length from target 0 ranging from 48 nucleotides to 70 nucleotides. FIG. 16 (left panel) provides the corresponding RNA editing percentages for each of the engineered guides with the first and second internal loops (e.g., guide 0.100.70 (−9, +33)), as well as controls (no transfection, GFP plasmid) via ADAR1. Shortened guides 0.92.62; 0.94.64; and 0.96.66 with a first internal loop and a second internal loop at −9, +33, respectively, demonstrated ADAR-mediated RNA editing percentages of about 46% or greater for each of biological replicates 1 and 2. The ADAR-mediated RNA editing percentages (38% and 40% for biological replicates 1 and 2, respectively) of the full length 0.100.70 guide (−9, +33). FIG. 17 illustrates the on-target editing percentages via ADAR1 for the different guide RNAs 0.92.62 (−9, +33) (top panel); 0.94.64 (−9, +33) (center panel); and 0.96.66 (−9, +33) (bottom panel). The ADAR-mediated RNA editing percentages of the aforementioned guides at target 0 were about 50%, about 45%, and about 45%, respectively.

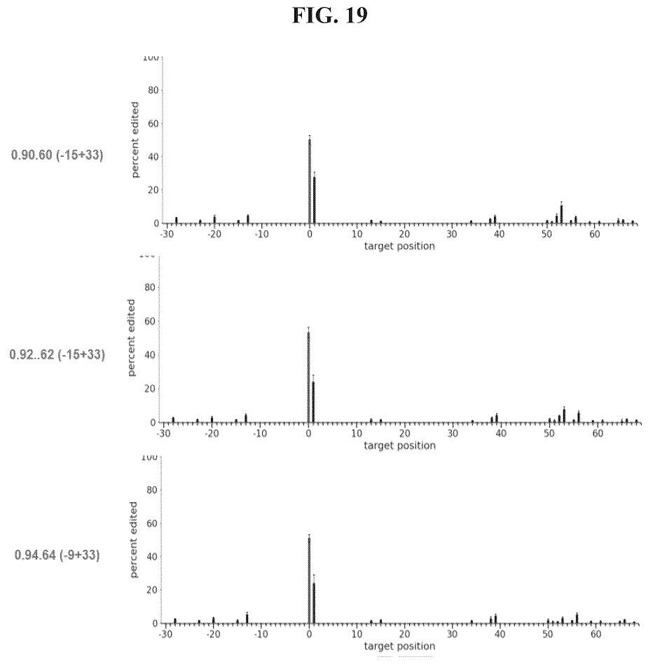

For example, the exemplary guides of FIG. 15 based on guide 0.100.70 (15, +33) represented in FIG. 18 (right panel) show that the 3′ end of the dsRNA had a length from target 0 ranging from 48 nucleotides to 70 nucleotides. FIG. 18 (left panel) provides the corresponding ADAR-mediated RNA editing percentages for each of the engineered guides with the first and second internal loops (e.g., guide 0.100.70 (−15, +33)), as well as controls (no transfection, GFP plasmid). Shortened guides 0.90.60; 0.92.62; and 0.94.64 with a first internal loop and a second internal loop at −15, +33, respectively, demonstrated ADAR-mediated RNA editing percentages of about 50% or greater for each of biological replicates 1 and 2. The ADAR-mediated RNA editing percentages (42% and 44% for biological replicates 1 and 2, respectively) of the full length 0.100.70 guide (−15, +33). FIG. 19 illustrates the on-target editing percentages for the different guide RNAs 0.90.60 (−15, +33) (top panel); 0.92.62 (−15, +33) (center panel); and 0.94.64 (−15, +33) (bottom panel)via ADAR1. The ADAR-mediated RNA editing percentages of the aforementioned guides at target 0 were each about 50%.

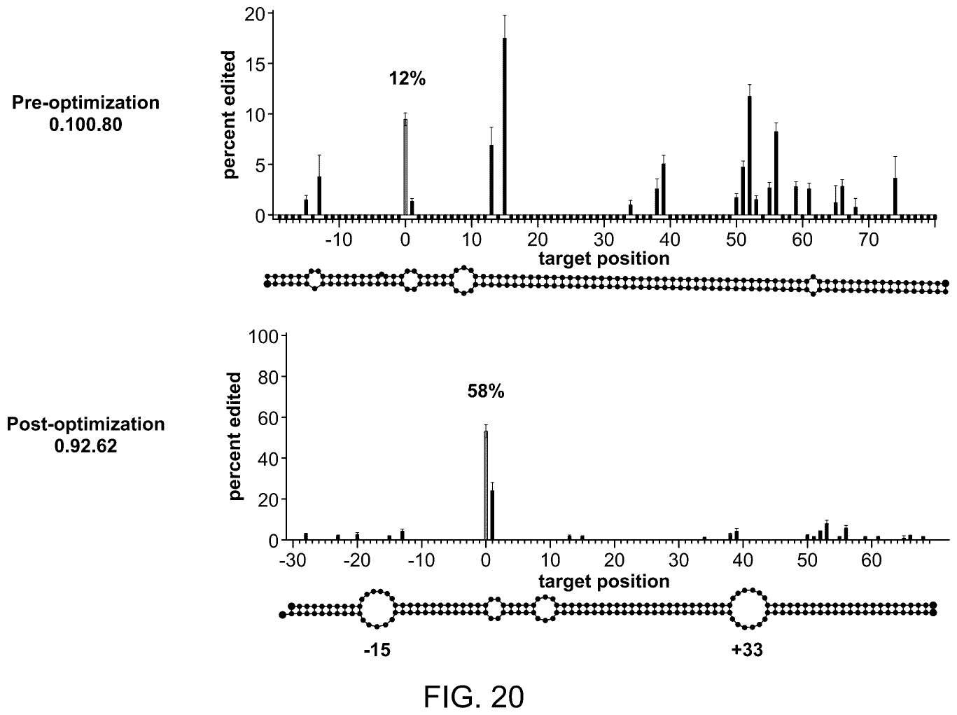

In order to determine the total cumulative effect of the superior left barbell, right barbell, and superior length, the ADAR-mediated editing efficiency of the superior guide with right and left barbells 0.92.62 (−15, +33) was compared to the 0.100.80 guide RNA comprising only the micro-footprint sequence. The top panel of FIG. 20 measured the on-target RNA editing percentage of about 12% for a guide 0.100.80 without first and second internal loops via ADAR1. The bottom panel measured the on-target RNA editing percentage of about 58% for a guide 0.92.62 with first and second internal loops (−15, +33) via ADAR1. Accordingly, the presence of the right barbell and left barbell imparted significant increases in on-target RNA editing via ADAR1 relative to the guide RNA lacking the barbells.

### Example 10

### Guide RNAs Against GAPDH

In order to demonstrate the general applicability of the barbell macro-footprint toward improving on target editing via ADAR, various guide RNAs were selected for targeted ADAR-mediated editing of GAPDH mRNA. Various guide RNAs designed to target GAPDH are depicted in FIG. 21, showing exemplary guide-target RNA complexes formed by engineered guide RNA. As a comparison, the superior guide RNA targeting ABCA4 with first and second internal loops (−9, +33) is presented first, followed by a guide RNA targeting GAPDH with only an A-C mismatch (GAPDH 100.70 A-C), a guide RNA targeting with a micro-footprint targeting GAPDH (GAPDH Shaker mimicry), and a guide RNA targeting GAPDH with a micro-footprint and a barbell macro-footprint with first and second internal loops (GAPDH Shaker mimicry −9, +33).

In FIG. 22, the GAPDH shaker mimicry guide RNA containing first and second internal loops at position −9 and +33, respectively (as selected in the superior guide RNA targeting ABCA4 with first and second internal loops), demonstrated a significant increase in the amount of ADAR-mediated RNA editing in the target GAPDH RNA (˜25% and ˜28% for biological replicates 1 and 2, respectively) relative to an otherwise comparable engineered guide RNA lacking the first internal loop and the second internal loop, e.g., Shaker mimicry (micro-footprint only) (6% for each of the biological replicates 1 and 2).

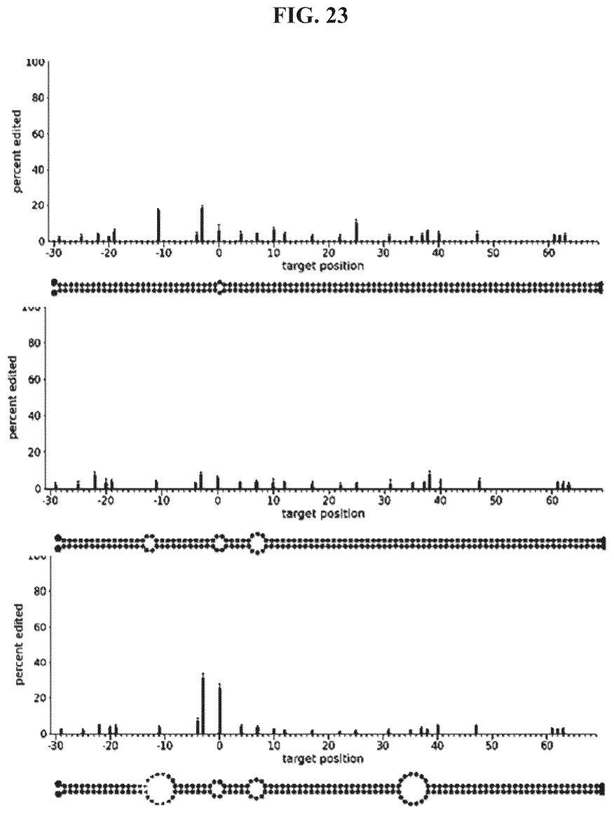

In FIG. 23, the GAPDH shaker mimicry guide RNA (bottom panel) containing first and second internal loops at positions −9 and +33, respectively, from the target 0 position, demonstrated that the presence of the first and second internal loops facilitated an increase in the amount of ADAR-mediated editing of the on-target adenosine at target 0 in the target GAPDH RNA (˜30%) relative to an otherwise comparable engineered guide RNA lacking the first internal loop and the second internal loop, e.g., Shaker mimicry (micro-footprint only) (center panel) (<10%) and GAPDH with the A-C mismatch (top panel) (<10%). Furthermore, the presence of the first and second internal loops improved editing efficiency and decreased the amount, percentage, or both the amount and percentage of off-target editing or off-target adenosine.

Accordingly, this example demonstrates that the superior barbell macro-footprint engineered for improved on-target editing of ABCA4 via ADAR in Example 9 also imparts improved on-target editing against GAPDH, a distinctly different target. Thus, the present example demonstrates the general applicability of utilizing a superior barbell macro-footprint to improve on target editing by a target RNA via an adenosine deaminase.

### Example 11

### Guide RNAs Against Rab7a

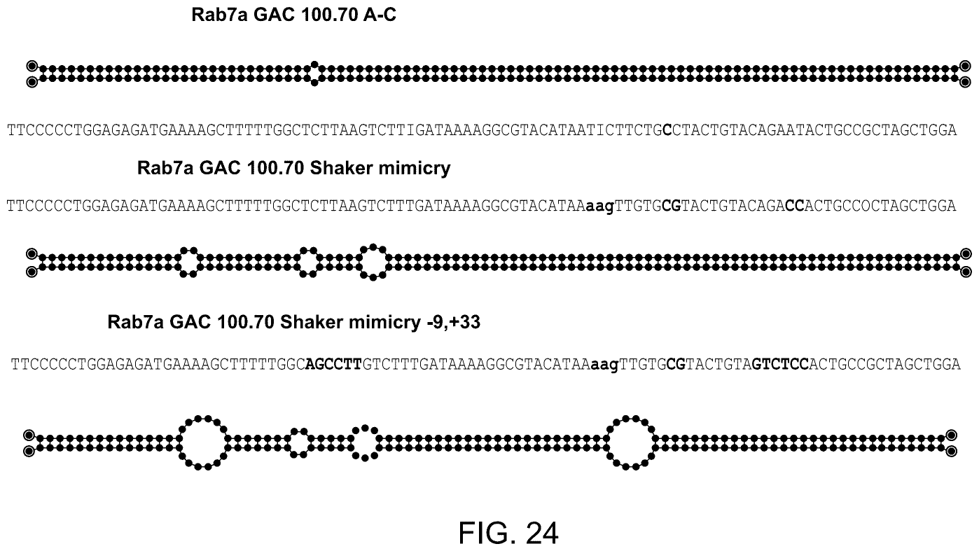

In order to further demonstrate the general applicability of the barbell macro-footprint toward improving on target editing, various guide RNAs were selected for targeted editing of Rab7a mRNA via ADAR. Various guide RNAs designed to target Rab7a GAC are depicted in FIG. 24, showing exemplary guide-target RNA complexes formed by engineered RNA guides. The first is a guide RNA with only an A-C mismatch (Rab7a 100.70 A-C; SEQ ID NO:109) (top panel), the second is a guide RNA with a micro-footprint targeting Rab7a, (Rab7a 100.70 Shaker mimicry; SEQ ID NO:110) (center panel), and the third is a guide RNA targeting Rab7a with a micro-footprint and a barbell macro-footprint with first and second internal loops (Rab7a Shaker mimicry −9,+33; SEQ ID NO:111) (bottom panel).

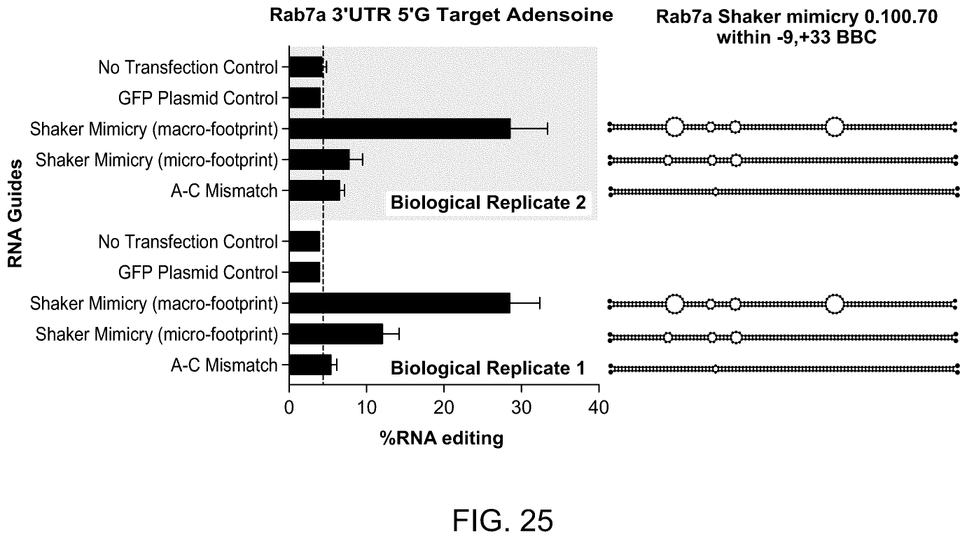

The Rab7a shaker mimicry guide RNA containing first and second internal loops at positions −9 and +33 (as selected in the superior guide RNA targeting ABCA4), respectively, from the target 0 position, in FIG. 25, demonstrated that the presence of the first and second internal loops facilitated an increase in the amount of RNA editing in the target Rab7a 3′UTR 5′G target adenosine RNA (˜28% for biological replicates 1 and 2) relative to an otherwise comparable engineered guide RNA lacking the first internal loop and the second internal loop, e.g., Shaker mimicry (micro-footprint) (12% and 7% for biological replicates 1 and 2, respectively).

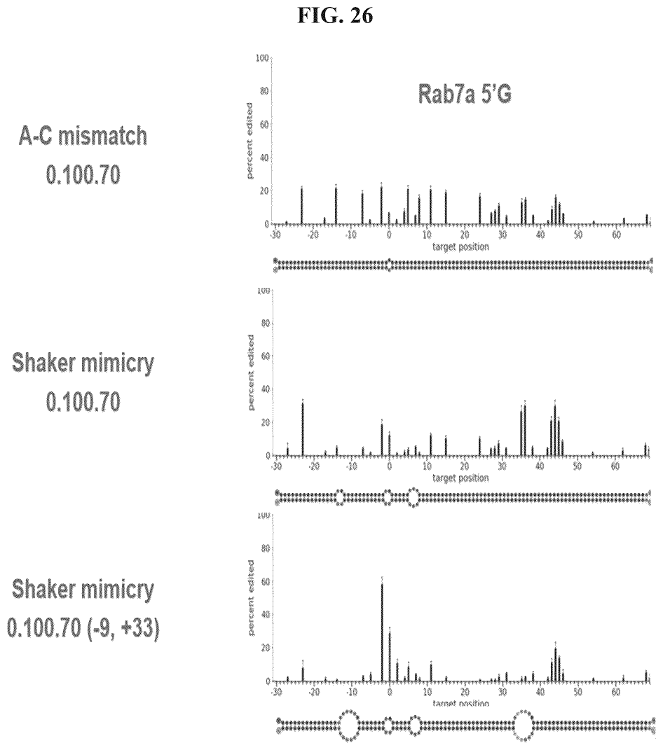

In FIG. 26, the Rab7a shaker mimicry guide RNA (bottom panel) containing first and second internal loops at position −9 and +33, respectively, from the target 0 position, demonstrated that the presence of the first and second internal loops facilitated an increase in the amount of editing of the on-target adenosine at target 0 in the target Rab7a RNA (˜30%) relative to an otherwise comparable engineered guide RNA lacking the first internal loop and the second internal loop, e.g., Shaker mimicry (micro-footprint) (center panel) (˜10%) and Rab7a with the A-C mismatch (top panel) (<10%). Furthermore, the presence of the first and second internal loops improved editing efficiency and decreased the amount, percentage, or both the amount and percentage of off-target editing or off-target adenosine.

Accordingly, this example demonstrates that the superior barbell macro-footprint engineered for improved on-target editing of ABCA4 in Example 9 also imparts improved on-target editing against Rab7a, a distinctly different target. Thus, the present example demonstrates the general applicability of utilizing a superior barbell macro-footprint to improve on target editing by a target RNA via an adenosine deaminase.

### Example 12

### Guide RNAs Against APP

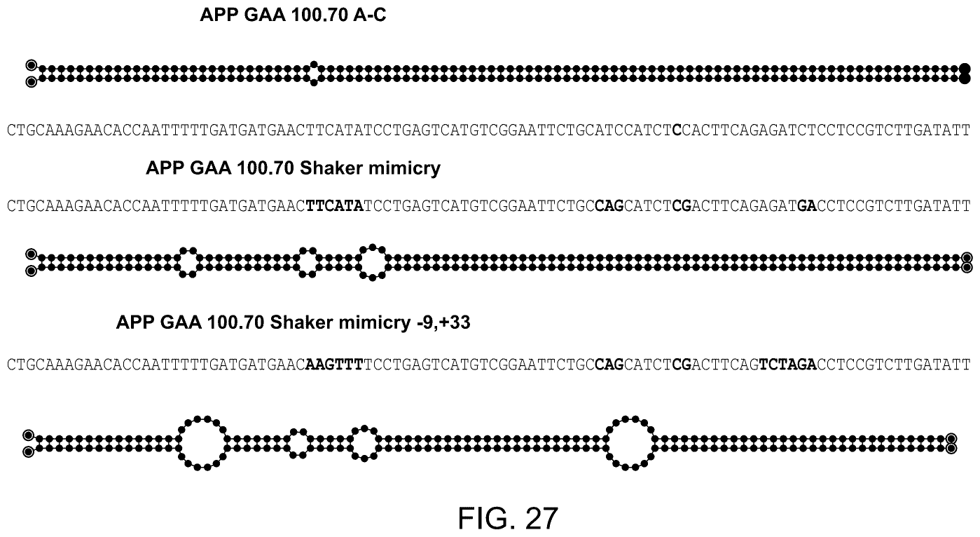

In order to further demonstrate the general applicability of the barbell macro-footprint toward improving on target editing, various guide RNAs were selected for targeted editing of APP mRNA via ADAR. Various constructs of the disclosure encoding a guide RNA designed to target APP GAA depicted in FIG. 27 show exemplary guide-target RNA complexes formed by engineered RNA guides. The first is a guide RNA with only an A-C mismatch (APP GAA 100.70 A-C; SEQ ID NO:112) (top panel), the second is a guide RNA with a micro-footprint targeting APP (APP GAA 100.70 Shaker mimicry; SEQ ID NO:113) (center panel), and the third is a guide RNA targeting APP GAA with a micro-footprint and a barbell macro-footprint with first and second internal loops (APP GAA 100.70 Shaker mimicry −9,+33; SEQ ID NO:114) (bottom panel).

The APP shaker mimicry guide RNA containing first and second internal loops at positions −9 and +33 (as selected in the superior guide RNA targeting ABCA4), respectively, from the target 0 position, in FIG. 28, demonstrated that the presence of the first and second internal loops facilitated an increase in the amount of RNA editing in the target APP 5′G target adenosine RNA (˜14% and ˜13% for biological replicates 1 and 2, respectively) relative to an otherwise comparable engineered guide RNA lacking the first internal loop and the second internal loop, e.g., Shaker mimicry (micro-footprint) (˜0.5% and ˜1% for biological replicates 1 and 2, respectively).

In FIG. 29, the APP 5′G shaker mimicry guide RNA 0.100.70 (bottom panel) containing first and second internal loops at position −9 and +33, respectively, from the target 0 position, demonstrated that the presence of the first and second internal loops facilitated an increase in the amount of editing of the on-target adenosine at target 0 in the target APP 5′G RNA (˜10%) via ADAR, relative to an otherwise comparable engineered guide RNA lacking the first internal loop and the second internal loop, e.g., Shaker mimicry (micro-footprint) (center panel) (˜1%) and APP 5′G with the A-C mismatch (top panel) (˜1%). Furthermore, the presence of the first and second internal loops improved editing efficiency and decreased the amount, percentage, or both the amount and percentage of off-target editing or off-target adenosine.

Accordingly, this example demonstrates that the superior barbell macro-footprint engineered for improved on-target editing of ABCA4 in Example 9 also imparts improved on-target editing against APP, a distinctly different target. Thus, the present example demonstrates the general applicability of utilizing a superior barbell macro-footprint to improve on target editing by a target RNA via an adenosine deaminase.

### Example 13

### Guide RNAs Against MAPT

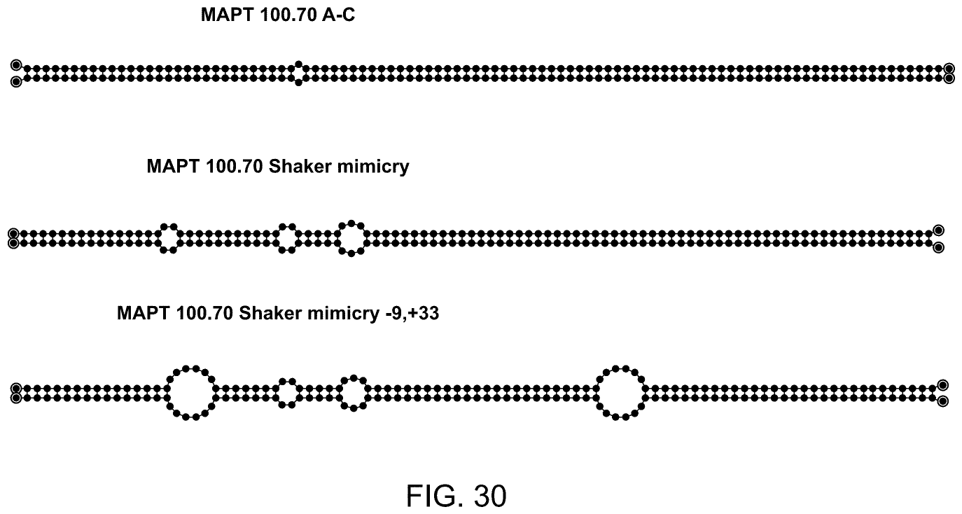

In order to further demonstrate the general applicability of the barbell macro-footprint toward improving on target editing, various guide RNAs were selected for targeted editing of MAPT mRNA via ADAR. Various constructs of the disclosure encoding a guide RNA designed to target MAPT 100.70 depicted in FIG. 30 show exemplary guide-target RNA complexes formed by engineered RNA guides. The first is a guide RNA with only an A-C mismatch (MAPT 100.70 A-C; SEQ ID NO:115) (top panel), the second is a guide RNA with the micro-footprint targeting MAPT (MAPT 100.70 Shaker mimicry; SEQ ID NO:116) (center panel), and the third is a guide RNA targeting MAPT with a micro-footprint and a barbell macro-footprint with first and second internal loops (MAPT 100.70 Shaker mimicry −9,+33; SEQ ID NO:117) (bottom panel).

The MAPT shaker mimicry guide RNA containing first and second internal loops at positions −9 and +33 (as selected in the superior guide RNA targeting ABCA4), respectively, from the target 0 position, in FIG. 31, demonstrated that the presence of the first and second internal loops facilitated an increase in the amount of RNA editing in the target MAPT 5′G target adenosine RNA (>40% for each of biological replicates 1 and 2) relative to an otherwise comparable engineered guide RNA lacking the first internal loop and the second internal loop, e.g., Shaker mimicry (micro-footprint) (˜5% for each of biological replicates 1 and 2). In FIG. 32, the MAPT 5′G TIS shaker mimicry guide RNA 0.100.70 (bottom panel) containing first and second internal loops at position −9 and +33, respectively, from the target 0 position, demonstrated that the presence of the first and second internal loops facilitated an increase in the amount of editing of the on-target adenosine at target 0 in the target MAPT 5′G TIS RNA (˜45%) relative to an otherwise comparable engineered guide RNA lacking the first internal loop and the second internal loop, e.g., Shaker mimicry (micro-footprint) (center panel) (˜5%) and MAPT 5′G TIS with the A-C mismatch (top panel) (˜5%). Furthermore, the presence of the first and second internal loops improved editing efficiency and decreased the amount, percentage, or both the amount and percentage of off-target editing or off-target adenosine.

Accordingly, this example demonstrates that the superior barbell macro-footprint engineered for improved on-target editing of ABCA4 in Example 9 also imparts improved on-target editing against MAPT, a distinctly different target. Thus, the present example demonstrates the general applicability of utilizing a superior barbell macro-footprint to improve on target editing by a target RNA via an adenosine deaminase.

### Example 14

High Throughput Screening of Engineered Guide RNAs Targeting LRRK2 mRNA

Using the compositions and methods described herein, high throughput screening (HTS) of long engineered guide RNAs (e.g., 100mer and longer) that target LRRK2 mRNA was performed, where said engineered guide RNAs form a micro-footprint comprised of various structural features in the guide-target RNA scaffold and form a barbell macro-footprint comprising two 6/6 internal loops near both ends of the guide-target RNA scaffold. Additionally, in this high throughput screen, self-annealing RNA structures were of a size (231 nucleotides) that allowed for screening for engineered guide RNAs that were 113 nucleotides in length, with the target adenosine to be edited positioned at the 57^{th} nucleotide. The high throughput screen was able to identify engineered guide RNAs that show high on-target adenosine editing (>60%, 30 min incubation with ADAR1 and ADAR2) and reduced to no local off-target adenosine editing (e.g., at the −2 position relative to the target adenosine to be edited, which is at position 0). Self-annealing RNA structures that formed a barbell macro-footprint in the guide-target RNA scaffold were screened to include 4 different micro-footprints (A/C mismatch (ATTCTACAGCAGTACTGAGCAATGCCGTAGTCAGCAATCTTTGCA (SEQ ID NO:168)), 2108 (ATTCTACGGCGGTACTGACCAATCCCGTAGTTAGCAATCTTTGCA (SEQ ID NO:169)), 871 (ATTCTACAGTAGGACTGAGCACTGCCGAGCTGGGCAATCTTT GCA (SEQ ID NO:170)), and 919 (CTTCTACAGCAGTTCGGAGGAATCCCGAGGTCAGCAATCTTTGCA (SEQ ID NO: 171))), tiling the position of the barbell macro-footprint from the −22 position to the −12 position at one end of the self-annealing RNA structure and from the +12 position to the +34 position at the other end of the self-annealing RNA structure. Self-annealing RNA structures comprising 1939 distinct guide RNA sequences and the sequences of the regions targeted by the guide RNAs were contacted with an RNA editing entity (e.g., a recombinant ADAR1 and/or ADAR2) for 30 minutes under conditions that allow for the editing of the regions targeted by the guide RNAs. The regions targeted by the guide RNAs were subsequently assessed for editing using next generation sequencing (NGS).

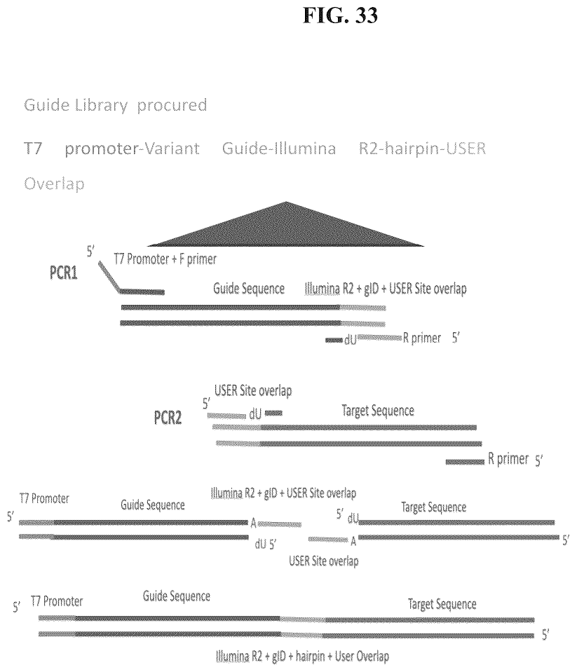

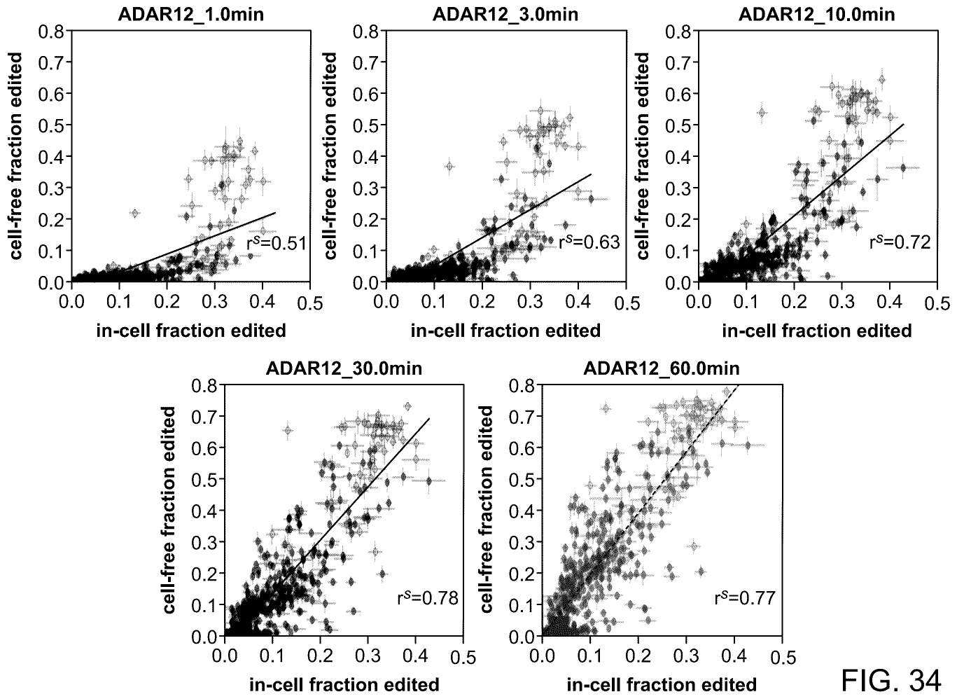

Libraries for screening of these longer engineered guides were generated as follows, and as summarized in FIG. 33: a candidate engineered guide library was procured having a construct with a T7 promoter, followed by the candidate engineered guide RNA sequence to be tested, followed by an Illumina R2 hairpin, followed by a sequence for a USER (Uracil-specific excision reagent) site Overlap. This library and the target sequence were PCR amplified, incorporating a deoxy-Uridine (dU) at the 3′ end of the constructs containing the candidate engineered guide RNA sequences and at the 5′ end of the target. Next, the PCR amplified library and target are incubated with the USER enzyme, resulting in nicking at the dU positions and ligation (using Taq ligase) of a given library construct containing the candidate engineered guide RNA sequence to the target sequence.

FIG. 34 shows a comparison of cell-free RNA editing using the methods and compositions described here versus in-cell RNA editing facilitated via the same engineered guide RNA sequence at various timepoints (1 min, 3 min, 10 min, 30 min, and 60 min). In this experiment, 40 candidate guide RNAs were screened. 50 nM ADAR1+100 nM ADAR2 was present in each cell. The editing values for each guide and the position of the adenosine that was edited is presented in FIG. 34 as a cumulative of 6 values. The open circles represent on target adenosine editing for each guide, whereas the black circles represent editing of adenosines other than the on-target adenosine (off target adenosines). As a whole, these data show that the cell-free high throughput screen is able to correlate well with in-cell RNA editing, in particular at certain timepoints (e.g., at 30 minutes).

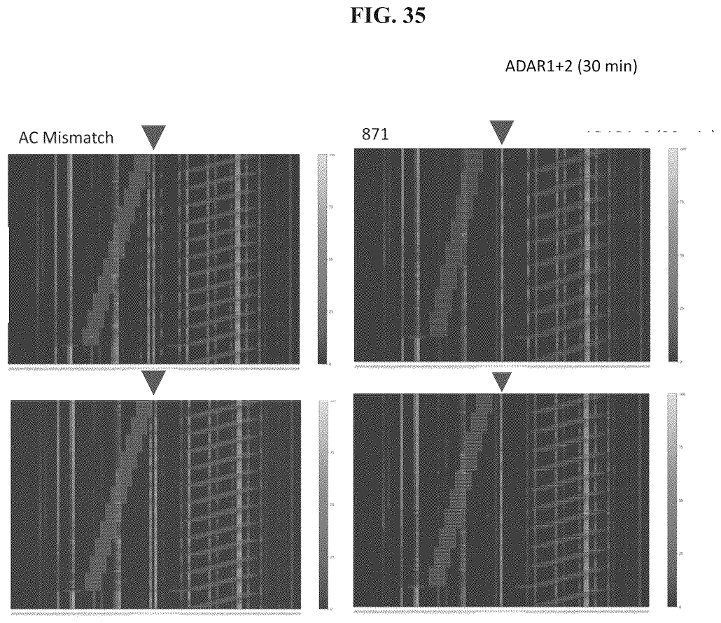

FIG. 35 shows heatmaps of all self-annealing RNA structures tested for the 4 micro-footprints described above formed within varying placement of a barbell macro-footprint. The y-axis shows all engineered guide RNAs tested and the x-axis shows the target sequence positions, with position 0 representing the target adenosine to be edited.

Exemplary engineered guide RNAs from the high throughput screen of this example are described in TABLE 3. While the engineered guide RNA sequences in TABLE 3 are provided as DNA sequences with a T substituted for each U, the corresponding RNA sequences are also encompassed herein. The candidate engineered guide RNAs of TABLE 3 showed specific editing of the A nucleotide at position 6055 of the mRNA encoding the LRRK2 G2019S. Percent on-target editing is calculated by the following formula: the number of reads containing “G” at the target/the total number of reads. Specificity is calculated by the following formula: (percent on target editing+100)/(sum of off target editing percentage at selected off-targets sites+100). The addition of barbells produced specific editing patterns. In particular, the presence of barbell at position −14 and position +26 appeared to increase the specificity of ADAR editing. Thus, specificity can be improved significantly through the combination of micro-footprint structural features and macro-footprint structural features such as barbells.

TABLE 3
Exemplary Guide RNAs that target LRRK2_ mRNA

| SEQ ID | | | Structural Features | |
|---|---|---|---|---|
| NO | ID | Sequence | (target/guide) | Metrics |
| SEQ ID | LRRK2_ | CCCTGGTGTGCCC | 1/1 U/G wobble base pair at −3 | ADAR1 on target: 0.38% |
| NO: 118 | bnPCR_ | TCTGATGTTTTTA | position; | ADAR1 specificity: 0.5 |
| | ID94725_ | TCCCCATTCTACA | 1/1 A/C mismatch at 0 position; | ADAR2 on target: 0.64% |
| | count351__ | GCGGTACTGAGC | 2_2/2 symmetric bulge at position | ADAR2 specificity: 0.37 |
| | NoLoops_ | AAATCCGTGGTCA | +2 (CA-AU); | ADAR1/2 on target: |
| | gID_02513 | GCAATCTTTGCAA | 1/1 U-G wobble base pair at +15 | 0.6% |
| | | TGATGGCAGCATT | | ADAR1/2 specificity: |
| | | GGGATACAGTGT | | 0.43 |
| | | GAAGAGCAGCA | | |
| SEQ ID | LRRK2_ | CCCTGGTGTGCCC | 6/6_symmetric internal loop at | ADAR1 on target: 0.32% |
| NO: 119 | bnPCR_ | TCTGATGTTTTTT | −14_position (UGCAAA- | ADAR1 specificity: 0.77 |
| | ID94725_ | AGGGGATTCTAC | AAACGU); | ADAR2 on target: 0.7% |
| | count351__ | AGCGGTACTGAG | 1/1_U/G wobble base pair at −3 | ADAR2 specificity: 0.72 |
| | −14_26_ | CAAATCCGTGGTC | position; | ADAR1/2 on target: |
| | gID_08570 | AGCAATCAAACG | 1/1 A/C mismatch at 0 position; | 0.64% |
| | | TATGATGGCAGC | 2/2 CA/AU symmetric bulge at | ADAR1/2 specificity: |
| | | ATTGGGATACAGT | +2_position; | 0.74 |
| | | GTGAAGAGCAGC | 1/1_U/G wobble at +15 position; | |
| | | A | 6/6 symmetric internal loop at +26 | |
| | | | position (GGGGAU-UAGGGG) | |
| SEQ ID | LRRK2_ | CCCTGGTGTGCCC | 1/1 C/C mismatch at −4 position; | ADAR1 on target: 0.43% |
| NO: 120 | bnPCR_ | TCTGATGTTTTTA | 1/1 (U/G) wobble base pair at −3 | ADAR1 specificity: 0.54 |
| | ID79791_ | TCCCCATTCTACA | position; | ADAR2 on target: 0.69% |
| | count610__ | TCTGTAGTGAGCA | 1/1 A/C mismatch at 0 position; | ADAR2 specificity: 0.38 |
| | NoLoops_ | ATTCCGTGCTCAG | 1/1 C/U mismatch at +2 position; | ADAR1/2 on target: |
| | gID_06724 | CAATCTTTGCAAT | 1/1 G/G mismatch at +11 | 0.66% |
| | | GATGGCAGCATT | position; 1/1 U/U mismatch at | ADAR1/2 specificity: |
| | | GGGATACAGTGT | +15 position; 1/1 C/U mismatch | 0.42 |
| | | GAAGAGCAGCA | at +17 position | |
| SEQ ID | LRRK2_ | CCCTGGTGTGCCC | 6/6 symmetric internal loop at −14 | ADAR1 on target: 0.21% |
| NO: 121 | bnPCR_ | TCTGATGTTTTTT | position (UGCAAA-AAACGU); | ADAR1 specificity: 0.77 |
| | ID79791_ | AGGGGATTCTAC | 1/1 C/C mismatch at −4 position; | ADAR2 on target: 0.66% |
| | count610__ | ATCTGTAGTGAGC | 1/1 U/G wobble base pair at −3 | ADAR2 specificity: 0.71 |
| | −14_26_ | AATTCCGTGCTCA | position; | ADAR1/2 on target: |
| | gID_09469 | GCAATCAAACGT | 1/1 A/C mismatch at 0 position; | 0.62% |
| | | ATGATGGCAGCA | 1/1 C/U mismatch at +2 position; | ADAR1/2 specificity: |
| | | TTGGGATACAGTG | 1/1 G/G mismatch at +11 position; | 0.75 |
| | | TGAAGAGCAGCA | 1/1 U/U mismatch +15 position; | |
| | | | 1/1 C/U mismatch at +17 position; | |
| | | | 6/6 symmetric internal loop at +26 | |
| | | | position (GGGGAU-UAGGGG) | |
| SEQ ID | LRRK2_ | CCCTGGTGTGCCC | 1/1 C/C mismatch at −4 position; | ADAR1 on target: 0.45% |
| NO: 122 | bnPCR_ | TCTGATGTTTTTA | 1/1 A/C mismatch at 0 position; | ADAR1 specificity: 0.57 |
| | ID66010_ | TCCCCATTCTACA | 1/1 C/C mismatch at +2 position; | ADAR2 on target: 0.67% |
| | count1326__ | CCTGGACTGAGA | 1/1 G/A mismatch at +6 position; | ADAR2 specificity: 0.39 |
| | NoLoops_ | AATCCCGTACTCA | 3/3 symmetric bulge at +13 | ADAR1/2 on target: |
| | gID_09247 | GCAATCTTTGCAA | position (ACU-UGG); | 0.65% |
| | | TGATGGCAGCATT | 1/1 C/C mismatch at +17 position | ADAR1/2 specificity: |
| | | GGGATACAGTGT | | 0.42 |
| | | GAAGAGCAGCA | | |
| SEQ ID | LRRK2_ | CCCTGGTGTGCCC | 6/6 symmetric internal loop at −14 | ADAR1 on target: 0.29% |
| NO: 123 | bnPCR_ | TCTGATGTTTTTT | position (UGCAAA-AAACGU); | ADAR1 specificity: 0.8 |
| | ID66010_ | AGGGGATTCTAC | 1/1 C/C mismatch at −4 position; | ADAR2 on target: 0.64% |
| | count1326__ | ACCTGGACTGAG | 1/1 A/C mismatch at 0 position; | ADAR2 specificity: 0.71 |
| | −14_26_ | AAATCCCGTACTC | 1/1 C/C mismatch at +2 position; | ADAR1/2 on target: |
| | gID_11327 | AGCAATCAAACG | 1/1 G/A mismatch at +6 position; | 0.62% |
| | | TATGATGGCAGC | 3/3 symmetric bulge at +13 | ADAR1/2 specificity: |
| | | ATTGGGATACAGT | position (ACU-UGG); | 0.76 |
| | | GTGAAGAGCAGC | 1/1 C/C mismatch at +17 position; | |
| | | A | 6/6_symmetric internal loop at +26 | |
| | | | position (GGGGAU-UAGGGG) | |
| SEQ ID | LRRK2_ | CCCTGGTGTGCCC | 1/1 U/G wobble base pair at −3 | ADAR1 on target: 0.5% |
| NO: 124 | bnPCR_ | TCTGATGTTTTTA | position; | ADAR1 specificity: 0.52 |
| | ID50437_ | TCCCCATTCTACA | 1/1 A/C mismatch at 0 position; | ADAR2 on target: 0.7% |
| | count708__ | GCAGTACGGTGC | 1/1 U/G wobble base pair at +4 | ADAR2 specificity: 0.38 |
| | NoLoops_ | AGTGCCGTGGTCA | position; | ADAR1/2 on target: |
| | gID_11520 | GCAATCTTTGCAA | 1/1 U/U mismatch at +8 position; | 0.65% |
| | | TGATGGCAGCATT | 1/1 A/G mismatch at +10 position | ADAR1/2 specificity: |
| | | GGGATACAGTGT | | 0.43 |
| | | GAAGAGCAGCA | | |
| SEQ ID | LRRK2_ | CCCTGGTGTGCCC | 6/6 symmetric internal loop at −14 | ADAR1 on target: 0.36% |
| NO: 125 | bnPCR_ | TCTGATGTTTTTT | position (UGCAAA-AAACGU); | ADAR1 specificity: 0.76 |
| | ID50437_ | AGGGGATTCTAC | 1/1 U/G wobble at −3 position; | ADAR2 on target: 0.68% |
| | count7081__ | AGCAGTACGGTG | 1/1 A/C mismatch at 0 position; | ADAR2 specificity: 0.68 |
| | −14_26_ | CAGTGCCGTGGTC | 1/1 U/G wobble base pair at +4 | ADAR1/2 on target: |
| | gID_09857 | AGCAATCAAACG | position; | 0.69% |
| | | TATGATGGCAGC | 1/1 U/U mismatch at +8 position; | ADAR1/2 specificity: |
| | | ATTGGGATACAGT | 1/1 A/G mismatch at +10 position; | 0.71 |
| | | GTGAAGAGCAGC | 6/6 symmetric internal loop at +26 | |
| | | A | position (GGGGAU-UAGGGG) | |
| SEQ ID | LRRK2_ | CCCTGGTGTGCCC | 1/1 U/G wobble base pair at −3 | ADAR1 on target: 0.4% |
| NO: 126 | bnPCR_ | TCTGATGTTTTTA | position; | ADAR1 specificity: 0.56 |
| | ID41799_ | TCCCCCTTCTACA | 1/1 A/C mismatch at 0 position; | ADAR2 on target: 0.69% |
| | count730__ | GCAGTAGTGAGTT | 2/2 symmetric bulge at +4 position | ADAR2 specificity: 0.37 |
| | NoLoops_ | TTGCCGTGGTCAG | (UU-UU); | ADAR1/2 on target: |
| | gID_04947 | CAATCTTTGCAAT | 1/1 G/U wobble base pair at +6 | 0.66% |
| | | GATGGCAGCATT | position; | ADAR1/2 specificity: |
| | | GGGATACAGTGT | 1/1 G/G mismatch at +11 position; | 0.41 |
| | | GAAGAGCAGCA | 1/1 U/C mismatch at +25 position | |
| SEQ ID | LRRK2_ | CCCTGGTGTGCCC | 6/6 symmetric internal loop at | ADAR1 on target: 0.3% |
| NO: 127 | bnPCR_ | TCTGATGTTTTTT | −14_position (UGCAAA- | ADAR1 specificity: 0.79 |
| | | AGGGGCTTCTACA | AAACGU); | ADAR2 on target: 0.68% |
| | ID41799_ | GCAGTAGTGAGTT | 1/1 U/G wobble base pair at −3 | ADAR2 specificity: 0.67 |
| | count7301__ | TTGCCGTGGTCAG | position; | ADAR1/2 on target: |
| | −14_26_ | CAATCAAACGTAT | 1/1 A/C mismatch at 0 position; | 0.65% |
| | gID_07755 | GATGGCAGCATT | 2/2 symmetric bulge at +4 position | ADAR1/2 specificity: |
| | | GGGATACAGTGT | (UU-UU);_ | 0.73 |
| | | GAAGAGCAGCA | 1/1 G/U wobble base pair at +6 | |
| | | | position; | |
| | | | 1/1 G/G mismatch at +11 position; | |
| | | | 7/7 symmetric internal loop at +25 | |
| | | | position (UGGGGAU- | |
| | | | UAGGGGC) | |
| SEQ ID | LRRK2_ | CCCTGGTGTGCCC | 1/1 C/A mismatch at −4 position; | ADAR1 on target: 0.45% |
| NO: 128 | bnPCR_ | TCTGATGTTTTTA | 1/1 U/G wobble base pair at −3 | ADAR1 specificity: 0.57 |
| | ID35277_ | TCCCCATTCTACA | position; | ADAR2 on target: 0.66% |
| | count295__ | GCAGTCCTGTACA | 1/1 A/C mismatch at 0 position; | ADAR2 specificity: 0.39 |
| | NoLoops_ | GTGCCGTGATCAG | 1/1 U/G wobble base pair at +4 | ADAR1/2 on target: |
| | gID_06840 | CAATCTTTGCAAT | position; | 0.64% |
| | | GATGGCAGCATT | 2/2 symmetric bulge at +7 position | ADAR1/2 specificity: |
| | | GGGATACAGTGT | (CU-UA); | 0.44 |
| | | GAAGAGCAGCA | 1/1 U/C mismatch at +12 position | |
| SEQ ID | LRRK2_ | CCCTGGTGTGCCC | 6/6 symmetric internal loop at −14 | ADAR1 on target: 0.27% |
| NO: 129 | bnPCR_ | TCTGATGTTTTTT | position (UGCAAA-AAACGU); | ADAR1 specificity: 0.76 |
| | ID35277_ | AGGGGATTCTAC | 1/1 C/A mismatch at −4 position; | ADAR2 on target: 0.68% |
| | count295__ | AGCAGTCCTGTAC | 1/1 U/G wobble base pair at −3 | ADAR2 specificity: 0.68 |
| | −14_26_ | AGTGCCGTGATCA | position; | ADAR1/2 on target: |
| | gID_09359 | GCAATCAAACGT | 1/1 A/C mismatch at 0 position; | 0.65% |
| | | ATGATGGCAGCA | 1/1 U/G wobble base pair at +4 | ADAR1/2 specificity: |
| | | TTGGGATACAGTG | position; | 0.71 |
| | | TGAAGAGCAGCA | 2/2 symmetric bulge at +7 position | |
| | | | (CU-UA); | |
| | | | 1/1 U/C mismatch at +12 position; | |
| | | | 6/6 symmetric internal loop at +26 | |
| | | | position (GGGGAU-UAGGGG | |
| SEQ ID | LRRK2_ | CCCTGGTGTGCCC | 6/6 symmetric internal loop at −10 | ADAR1 on target: 0.33% |
| NO: 130 | bnPCR_ | TCTGATGTTTTTA | position (AAGAUU-CUAGGC); | ADAR1 specificity: 0.7 |
| | ID34601_ | TCCCCATTCGCCT | 1/1 G/U wobble base pair at −6 | ADAR2 on target: 0.63% |
| | count2108__ | GAGGTACTGACC | position; | ADAR2 specificity: 0.52 |
| | −10_16_ | AATCCCGTAGTTA | 1/1 A/C mismatch at 0 position; | ADAR1/2 on target: |
| | gID_01444 | GCCTAGGCTGCA | 1/1 C/C mismatch at +2 position; | 0.6% |
| | | ATGATGGCAGCA | 1/1 C/C mismatch at +7 position; | ADAR1/2 specificity: |
| | | TTGGGATACAGTG | 1/1 U/G wobble base pair at +15 | 0.58 |
| | | TGAAGAGCAGCA | position; | |
| | | | 6/6 symmetric internal loop at +16 | |
| | | | position (GCUGUA-GCCUGA) | |
| SEQ ID | LRRK2_ | CCCTGGTGTGCCC | 6/6 symmetric internal loop at −22 | ADAR1 on target: 0.22% |
| NO: 131 | bnPCR_ | TCTGATGTTTTTC | position (GCCAUC-AACCUG); | ADAR1 specificity: 0.73 |
| | ID33405_ | TACAGATTCTACA | 1/1 C/C mismatch at −8 position; | ADAR2 on target: 0.61% |
| | count860__ | GCAGTACAGAGG | 1/1 U/G wobble base pair at −3 | ADAR2 specificity: 0.56 |
| | −22_26_ | ACTGCCGAGGTC | position; | ADAR1/2 on target: |
| | gID_00318 | ACCAATCTTTGCA | 1/1 A/A mismatch at −2 position; | 0.56% |
| | | ATAACCTGAGCAT | 1/1 A/C mismatch at 0 position; | ADAR1/2 specificity: |
| | | TGGGATACAGTGT | 1/1 U/C mismatch at +4 position; | 0.63 |
| | | GAAGAGCAGCA | 1/1 G/G mismatch at +6 position; | |
| | | | 1/1 A/A mismatch at +10 position; | |
| | | | 2/2 symmetric bulge at +26 | |
| | | | position (GG-AG); | |
| | | | 3/3 symmetric bulge at +29 | |
| | | | position (GAU-CUA) | |
| SEQ ID | LRRK2_ | CCCTGGTGTGCCC | 6/6 symmetric internal loop at −20 | ADAR1 on target: 0.22% |
| NO: 132 | bnPCR_ | TCTGATGTTTTTT | position (CAUCAU-UACUAC); | ADAR1 specificity: 0.73 |
| | ID33405_ | AGGGGATTCTAC | 1/1 C/C mismatch at −8 position; | ADAR2 on target: 0.63% |
| | count860__ | AGCAGTACAGAG | 1/1 U/G wobble base pair at −3 | ADAR2 specificity: 0.58 |
| | −20_26_ | GACTGCCGAGGT | position; | ADAR1/2 on target: |
| | gID_09192 | CACCAATCTTTGC | 1/1 A/A mismatch at −2 position; | 0.58% |
| | | ATACTACGCAGC | 1/1 A/C mismatch at 0 position; | ADAR1/2 specificity: |
| | | ATTGGGATACAGT | 1/1 U/C mismatch at +4 position; | 0.64 |
| | | GTGAAGAGCAGC | 1/1 G/G mismatch at +6 position; | |
| | | A | 1/1 A/A mismatch at +10 position; | |
| | | | 6/6 symmetric internal loop at | |
| | | | +26 position (GGGGAU- | |
| | | | UAGGGG) | |
| SEQ ID | LRRK2_ | CCCTGGTGTGCCC | 6/6 symmetric internal loop at −16 | ADAR1 on target: 0.21% |
| NO: 133 | bnPCR_ | TCTGATGTTTTTC | position (AUUGCA-GUCUAG); | ADAR1 specificity: 0.76 |
| | ID33405_ | GGGAGATTCTAC | 1/1 C/C mismatch at −8 position; | ADAR2 on target: 0.66% |
| | count860__ | AGCAGTACAGAG | 1/1 U/G wobble at −3 position; | ADAR2 specificity: 0.6 |
| | −16_26_ | GACTGCCGAGGT | 1/1 A/A mismatch at −2 position; | ADAR1/2 on target: |
| | gID_11898 | CACCAATCTTGTC | 1/1 A/C mismatch at 0 position; | 0.59% |
| | | TAGGATGGCAGC | 1/1 U/C mismatch at +4 position; | ADAR1/2 specificity: |
| | | ATTGGGATACAGT | 1/1 G/G mismatch at +6 position; | 0.63 |
| | | GTGAAGAGCAGC | 1/1 A/A mismatch at +10 position; | |
| | | A | 6/6 symmetric internal loop at | |
| | | | +26 position (GGGGAU- | |
| | | | CGGGAG) | |
| SEQ ID | LRRK2_ | CCCTGGTGTGCCC | 6/6 symmetric internal loop at −12 | ADAR1 on target: 0.39% |
| NO: 134 | bnPCR_ | TCTGATGTATGTT | position (CAAAGA-CGCUAA); | ADAR1 specificity: 0.75 |
| | ID33405_ | CCCCCATTCTACA | 1/1 C/C mismatch at −8 position; | ADAR2 on target: 0.45% |
| | count860__ | GCAGTACAGAGG | 1/1 U/G wobble base pair at −3 | ADAR2 specificity: 0.55 |
| | −12_30_ | ACTGCCGAGGTC | position; | ADAR1/2 on target: |
| | gID_05698 | ACCAACGCTAAC | 1/1 A/A mismatch at −2 position; | 0.5% |
| | | AATGATGGCAGC | 1/1 A/C mismatch at 0 position; | ADAR1/2 specificity: |
| | | ATTGGGATACAGT | 1/1 U/C mismatch at +4 position; | 0.66 |
| | | GTGAAGAGCAGC | 1/1 G/G mismatch at +6 position; | |
| | | A | 1/1 A/A mismatch at +10 position; | |
| | | | 4/4 symmetric bulge at +30 | |
| | | | position (AUAA-GUUC); | |
| | | | 1/1 A/A mismatch at +35 position | |
| SEQ ID | LRRK2_ | CCCTGGTGTGCCC | 6/6 symmetric internal loop at −8 | ADAR1 on target: 0.33% |
| NO: 135 | bnPCR_ | TCTGATGTTTTTA | position (GAUUGC-CUUUGA); | ADAR1 specificity: 0.67 |
| | ID33405_ | TGGAAAGTCTAC | 1/1 U/G wobble base pair at −3 | ADAR2 on target: 0.58% |
| | count860__ | AGCAGTACAGAG | position; | ADAR2 specificity: 0.58 |
| | −8_24_ | GACTGCCGAGGT | 1/1 A/A mismatch at −2 position; | ADAR1/2 on target: |
| | gID_05952 | CACTTTGATTTGC | 1/1 A/C mismatch at 0 position; | 0.49% |
| | | AATGATGGCAGC | 1/1 U/C mismatch at +4 position; | ADAR1/2 specificity: |
| | | ATTGGGATACAGT | 1/1 G/G mismatch at +6 position; | 0.64 |
| | | GTGAAGAGCAGC | 1/1 A/A mismatch at +10 position; | |
| | | A | 6/6 symmetric internal loop at +24 | |
| | | | position (AUGGGG-GGAAAG) | |
| SEQ ID | LRRK2_ | CCCTGGTGTGCCC | 6/6 symmetric internal loop at −22 | ADAR1 on target: 0.25% |
| NO: 136 | bnPCR_ | TCTGATGTTTTTC | position (GCCAUC-CAAAAA); | ADAR1 specificity: 0.72 |
| | ID29209_ | GAAGAATTCTAC | 1/1 U/G wobble base pair at −7 | ADAR2 on target: 0.59% |
| | count871__ | AGTAGGACTGAG | position; | ADAR2 specificity: 0.61 |
| | −22_26_ | CACTGCCGAGCTG | 1/1 G/G mismatch at −6 position; | ADAR1/2 on target: |
| | gID_01244 | GGCAATCTTTGCA | 0/1 asymmetric bulge at −4 position | 0.56% |
| | | ATCAAAAAAGCA | (-C); | ADAR1/2 specificity: |
| | | TTGGGATACAGTG | 1/0 asymmetric bulge at −2 position | 0.67 |
| | | TGAAGAGCAGCA | (A-); | |
| | | | 1/1 A/C mismatch at 0 position; | |
| | | | 1/1 U/C mismatch at +4 position; | |
| | | | 1/1 A/G mismatch at +13 position; | |
| | | | 1/1 G/U wobble at +16 position; | |
| | | | 6/6 symmetric internal loop at +26 | |
| | | | position (GGGGAU-CGAAGA) | |
| SEQ ID | LRRK2_ | CCCTGGTGTGCCC | 6/6 symmetric internal loop at −20 | ADAR1 on target: 0.25% |
| NO: 137 | bnPCR_ | TCTGATGTTTTTT | position (CAUCAU-UACUAC); | ADAR1 specificity: 0.73 |
| | ID29209_ | AGGGGATTCTAC | 1/1 U/G wobble base pair at −7 | ADAR2 on target: 0.62% |
| | count871__ | AGTAGGACTGAG | position; | ADAR2 specificity: 0.67 |
| | −20_26_ | CACTGCCGAGCTG | 1/1 G/G mismatch at −6 position; | ADAR1/2 on target: |
| | gID_07422 | GGCAATCTTTGCA | 0/1 asymmetric bulge at −4 position | 0.6% |
| | | TACTACGCAGCAT | (-C); | ADAR1/2 specificity: |
| | | TGGGATACAGTGT | 1/0 asymmetric bulge at −2 position | 0.69 |
| | | GAAGAGCAGCA | (A-); | |
| | | | 1/1 A/C mismatch at 0 position; | |
| | | | 1/1 U/C mismatch at +4 position; | |
| | | | 1/1 A/G mismatch at +13 position; | |
| | | | 1/1 G/U wobble base pair at +16 | |
| | | | position; | |
| | | | 6/6 symmetric internal loop at +26 | |
| | | | position (GGGGAU-UAGGGG) | |
| SEQ ID | LRRK2_ | CCCTGGTGTGCCC | 6/6 symmetric internal loop at −16 | ADAR1 on target: 0.32% |
| NO: 138 | bnPCR_ | TCTGATGTTTTTC | position (AUUGCA-CAUUUG); | ADAR1 specificity: 0.77 |
| | ID29209_ | TACAGATTCTACA | 1/1 U/G wobble base pair at −7 | ADAR2 on target: 0.6% |
| | count8711__ | GTAGGACTGAGC | position; | ADAR2 specificity: 0.66 |
| | −16_26_ | ACTGCCGAGCTG | 1/1 G/G mismatch at −6 position; | ADAR1/2 on target: |
| | gID_01312 | GGCAATCTTCATT | 0/1 aysmmetric bulge at −4 position | 0.57% |
| | | TGGATGGCAGCA | (-C); | ADAR1/2 specificity: |
| | | TTGGGATACAGTG | 1/0 asymmetric bulge at −2 position | 0.69 |
| | | TGAAGAGCAGCA | (A-); | |
| | | | 1/1 A/C mismatch at 0 position; | |
| | | | 1/1 U/C mismatch at +4 position; | |
| | | | 1/1 A/G mismatch at +13 position; | |
| | | | 1/1 G/U wobble base pair at +16 | |
| | | | position; | |
| | | | 2/2 symmetric bulge at +26 | |
| | | | position (GG-AG); | |
| | | | 3/3 symmetric bulge at +29 | |
| | | | position (GAU-CUA) | |
| SEQ ID | LRRK2_ | CCCTGGTGTGCCC | 6/6 symmetric internal loop at −16 | ADAR1 on target: 0.28% |
| NO: 139 | bnPCR_ | TCTGATGTTTTTA | position (AUUGCA-GAGUCG); | ADAR1 specificity: 0.81 |
| | ID29209_ | TGGGGTGTCTACA | 1/1 U/G wobble base pair at −7 | ADAR2 on target: 0.62% |
| | count871__ | GTAGGACTGAGC | position; | ADAR2 specificity: 0.73 |
| | −16_24_ | ACTGCCGAGCTG | 1/1 G/G mismatch at −6 position; | ADAR1/2 on target: |
| | gID_00727 | GGCAATCTTGAGT | 0/1 asymmetric bulge at −4 position | 0.58% |
| | | CGGATGGCAGCA | (-C); | ADAR1/2 specificity: |
| | | TTGGGATACAGTG | 1/0 asymmetric bulge at −2 position | 0.76 |
| | | TGAAGAGCAGCA | (A-); | |
| | | | 1/1 A/C mismatch at 0 position; | |
| | | | 1/1 U/C mismatch at +4 position; | |
| | | | 1/1 A/G mismatch at +13 position; | |
| | | | 1/1 G/U wobble at +16 position; | |
| | | | 6/6 symmetric internal loop at | |
| | | | +24 position (AUGGGG- | |
| | | | GGGGUG) | |
| SEQ ID | LRRK2_ | CCCTGGTGTGCCC | 6/6 symmetric internal loop at −16 | ADAR1 on target: 0.2% |
| NO: 140 | bnPCR_ | TCTGATGTTTTTA | position (AUUGCA-CUCCCA); | ADAR1 specificity: 0.81 |
| | ID29209_ | TCCGAAACATAC | 1/1 U/G wobble at −7 position; | ADAR2 on target: 0.55% |
| | count871__ | AGTAGGACTGAG | 1/1 G/G mismatch at −6 positon; | ADAR2 specificity: 0.74 |
| | −16_22_ | CACTGCCGAGCTG | 0/1 asymmetric bulge at −4 position | ADAR1/2 on target: |
| | gID_11427 | GGCAATCTTCTCC | (-C); | 0.48% |
| | | CAGATGGCAGCA | 1/0 asymmetric bulge at −2 position | ADAR1/2 specificity: |
| | | TTGGGATACAGTG | (A-); | 0.75 |
| | | TGAAGAGCAGCA | 1/1 A/C mismatch at 0 position; | |
| | | | 1/1 U/C mismatch at +4 position; | |
| | | | 1/1 A/G mismatch at +13 position; | |
| | | | 1/1 G/U wobble base pair at +16 | |
| | | | position; | |
| | | | 6/6 symmetric internal loop at +22 | |
| | | | position (GAAUGG-GAAACA) | |
| SEQ ID | LRRK2_ | CCCTGGTGTGCCC | 6/6 symmetric internal loop at −10 | ADAR1 on target: 0.45% |
| NO: 141 | bnPCR_ | TCTGATGTTTTTC | position (AAGAUU-CUCAGG); | ADAR1 specificity: 0.78 |
| | ID29209_ | ATGAGATTCTACA | 1/1 U/G wobble base pair at −7 | ADAR2 on target: 0.64% |
| | count871__ | GTAGGACTGAGC | position; | ADAR2 specificity: 0.7 |
| | −10_26_ | ACTGCCGAGCTG | 1/1 G/G mismatch at −6 position; | ADAR1/2 on target: |
| | gID_00091 | GGCCTCAGGTGC | 0/1 asymmetric bulge at −4 position | 0.59% |
| | | AATGATGGCAGC | (-C); | ADAR1/2 specificity: |
| | | ATTGGGATACAGT | 1/0 asymmetric bulge at −2 position | 0.73 |
| | | GTGAAGAGCAGC | (A-); | |
| | | A | 1/1 A/C mismatch at 0 position; | |
| | | | 1/1 U/C mismatch at +4 position; | |
| | | | 1/1 A/G mismatch at +13 position; | |
| | | | 1/1 G/U wobble base pair at +16 | |
| | | | position; | |
| | | | 6/6 symmetric internal loop at +26 | |
| | | | position (GGGGAU-CAUGAG) | |
| SEQ ID | LRRK2_ | CCCTGGTGTGCCC | 4/4 symmetric bulge at −9 position | ADAR1 on target: 0.38% |
| NO: 142 | bnPCR_ | TCTGATGTTTTTT | (AUUG-GUUC); | ADAR1 specificity: 0.74 |
| | ID29209_ | ACAGAATTCTACA | 1/1 U/G wobble base pair at −7 | ADAR2 on target: 0.57% |
| | count871__ | GTAGGACTGAGC | position; | ADAR2 specificity: 0.65 |
| | −8_26_ | ACTGCCGAGCTG | 1/1 G/G mismatch at −6 position; | ADAR1/2 on target: |
| | gID_11298 | GGGTTCCTTTGCA | 0/1 asymmetric bulge at −4 position | 0.53% |
| | | ATGATGGCAGCA | (-C); | ADAR1/2 specificity: |
| | | TTGGGATACAGTG | 1/0 asymmetric bulge at −2 position | 0.71 |
| | | TGAAGAGCAGCA | (A-); | |
| | | | 1/1 A/C mismatch at 0 position; | |
| | | | 1/1 U/C mismatch at +4 position; | |
| | | | 1/1 A/G mismatch at +13 position; | |
| | | | 1/1 G/U wobble base pair at +16 | |
| | | | position; | |
| | | | 6/6 symmetric internal loop at +26 | |
| | | | position (GGGGAU-UACAGA) | |
| SEQ ID | LRRK2_ | CCCTGGTGTGCCC | 6/6 symmetric internal loop at −20 | ADAR1 on target: 0.39% |
| NO: 143 | bnPCR_ | TCTGATGTTTTTT | position (CAUCAU-UACUAC); | ADAR1 specificity: 0.71 |
| | ID28134_ | AGGGGATTCTACC | 1/1 A/C mismatch at 0 position; | ADAR2 on target: 0.7% |
| | count1700__ | GCAGTACTACCCG | 1/1 C/C mismatch at +2 position; | ADAR2 specificity: 0.64 |
| | −20_26_ | ATCCCGTAGTCAG | 1/1 U/G wobble base pair at +5 | ADAR1/2 on target: |
| | gID_06283 | CAATCTTTGCATA | position; | 0.56% |
| | | CTACGCAGCATTG | 3/3 symmetric bulge at +7 position | ADAR1/2 specificity: 0.7 |
| | | GGATACAGTGTG | (CUC-ACC); | |
| | | AAGAGCAGCA | 1/1 U/C mismatch at +18 position; | |
| | | | 6/6 symmetric internal loop at +26 | |
| | | | position (GGGGAU-UAGGGG) | |
| SEQ ID | LRRK2_ | CCCTGGTGTGCCC | 1/1 U/G wobble base pair at −7 | ADAR1 on target: 0.44% |
| NO: 144 | bnPCR_ | TCTGATGTTTTTA | position; | ADAR1 specificity: 0.65 |
| | ID24310_ | TCCCCATTCTAGG | 1/1 G/G mismatch at −6 position; | ADAR2 on target: 0.7% |
| | count1321__ | GCAGTAGGGTGC | 1/1 U/G wobble base pair at −3 | ADAR2 specificity: 0.39 |
| | NoLoops_ | ACTGCCGTGGTGG | position; | ADAR1/2 on target: |
| | gID_11060 | GCAATCTTTGCAA | 1/1 A/C mismatch at 0 position; | 0.68% |
| | | TGATGGCAGCATT | 1/1 U/C mismatch at +4 position; | ADAR1/2 specificity: |
| | | GGGATACAGTGT | 1/1 U/U mismatch at +8 position; | 0.46 |
| | | GAAGAGCAGCA | 2/2 symmetric bulge at +10 | |
| | | | position (AG-GG); | |
| | | | 1/1 U/G wobble base pair at +18 | |
| | | | position; | |
| | | | 1/1 G/G mismatch at +19 position | |
| SEQ ID | LRRK2_ | CCCTGGTGTGCCC | 6/6 symmetric internal loop at −14 | ADAR1 on target: 0.15% |
| NO: 145 | bnPCR_ | TCTGATGTTTTTT | position (UGCAAA-AAACGU); | ADAR1 specificity: 0.82 |
| | ID24310_ | AGGGGATTCTAG | 1/1 U/G wobble base pair at −7 | ADAR2 on target: 0.67% |
| | count1321__ | GGCAGTAGGGTG | position; | ADAR2 specificity: 0.72 |
| | −14_26_ | CACTGCCGTGGTG | 1/1 G/G mismatch at −6 position; | ADAR1/2 on target: |
| | gID_02264 | GGCAATCAAACG | 1/1 U/G wobble base pair at −3 | 0.64% |
| | | TATGATGGCAGC | position; | ADAR1/2 specificity: |
| | | ATTGGGATACAGT | 1/1 A/C mismatch at 0 position; | 0.75 |
| | | GTGAAGAGCAGC | 1/1 U/C mismatch at +4 position; | |
| | | A | 1/1 U/U mismatch at +8 position; | |
| | | | 2/2 symmetric bulge at +10 | |
| | | | position (AG-GG); | |
| | | | 1/1 U/G wobble at +18 position; | |
| | | | 1/1 G/G mismatch at +19 position; | |
| | | | 6/6 symmetric internal loop at +26 | |
| | | | position (GGGGAU-UAGGGG) | |
| SEQ ID | LRRK2_ | CCCTGGTGTGCCC | 1/1 U/G wobble base pair at −3 | ADAR1 on target: 0.56% |
| NO: 146 | bnPCR_ | TCTGATGTTTTTA | position; | ADAR1 specificity: 0.59 |
| | ID23393_ | TCCCCATTCTACC | 1/1 A/C mismatch at 0 position; | ADAR2 on target: 0.69% |
| | count2397__ | GCTGTGCTGGGCA | 1/1 C/C mismatch at +2 position; | ADAR2 specificity: 0.41 |
| | NoLoops_ | ATCCCGTGGTCAG | 1/1 U/G wobble base pair at +8 | ADAR1/2 on target: |
| | gID_09625 | CAATCTTTGCAAT | position; | 0.67% |
| | | GATGGCAGCATT | 1/1 U/G wobble base pair at +12 | ADAR1/2 specificity: |
| | | GGGATACAGTGT | position; | 0.45 |
| | | GAAGAGCAGCA | 1/1 U/U mismatch at +15 position; | |
| | | | 1/1 U/C mismatch at +18 position | |
| SEQ ID | LRRK2_ | CCCTGGTGTGCCC | 6/6 symmetric internal loop at −20 | ADAR1 on target: 0.41% |
| NO: 147 | bnPCR_ | TCTGATGTTTTTT | position (CAUCAU-UACUAC); | ADAR1 specificity: 0.67 |
| | ID23393_ | AGGGGATTCTACC | 1/1 U/G wobble base pair at −3 | ADAR2 on target: 0.64% |
| | count2397__ | GCTGTGCTGGGCA | position; | ADAR2 specificity: 0.7 |
| | 2-0_26_ | ATCCCGTGGTCAG | 1/1 A/C mismatch at 0 position; | ADAR1/2 on target: |
| | gID_06774 | CAATCTTTGCATA | 1/1 C/C mismatch at +2 position; | 0.63% |
| | | CTACGCAGCATTG | 1/1 U/G wobble base pair at +8 | ADAR1/2 specificity: |
| | | GGATACAGTGTG | position; | 0.73 |
| | | AAGAGCAGCA | 1/1 U/G wobble base pair at +12 | |
| | | | position; | |
| | | | 1/1 U/U mismatch at +15 position; | |
| | | | 1/1 U/C mismatch at +18 position; | |
| | | | 6/6 symmetric internal loop at +26 | |
| | | | position (GGGGAU-UAGGGG) | |
| SEQ ID | LRRK2_ | CCCTGGTGTGCCC | 6/6 symmetric internal loop at −14 | ADAR1 on target: 0.39% |
| NO: 148 | bnPCR_ | TCTGATGTTTTTT | position (UGCAAA-AAACGU); | ADAR1 specificity: 0.83 |
| | ID23393_ | AGGGGATTCTACC | 1/1 U/G wobble base pair at −3 | ADAR2 on target: 0.67% |
| | count2397__ | GCTGTGCTGGGCA | position; | ADAR2 specificity: 0.75 |
| | −14_26_ | ATCCCGTGGTCAG | 1/1 A/C mismatch at 0 position; | ADAR1/2 on target: |
| | gID_06456 | CAATCAAACGTAT | 1/1 C/C mismatch at +2 position; | 0.62% |
| | | GATGGCAGCATT | 1/1 U/G wobble base pair at +8 | ADAR1/2 specificity: |
| | | GGGATACAGTGT | position; | 0.75 |
| | | GAAGAGCAGCA | 1/1 U/G wobble base pair at +12 | |
| | | | position; | |
| | | | 1/1 U/G mismatch at +15 position; | |
| | | | 1/1 U/C mismatch at +18 position; | |
| | | | 6/6 symmetric internal loop at +26 | |
| | | | position (GGGGAU-UAGGGG) | |
| SEQ ID | LRRK2_ | CCCTGGTGTGCCC | 1/1 U/G wobble at −3 position; | ADAR1 on target: 0.48% |
| NO: 149 | bnPCR_ | TCTGATGTTTTTA | 1/1 A/C mismatch at 0 position; | ADAR1 specificity: 0.5 |
| | ID22759_ | TCCCCATTCTACA | 1/1 U/G wobble base pair at +4 | ADAR2 on target: 0.68% |
| | count1590__ | ACAGTACGGTGA | position; | ADAR2 specificity: 0.43 |
| | NoLoops_ | AGTGCCGTGGTCA | 5/5 symmetric internal loop at +6 | ADAR1/2 on target: |
| | gID_06335 | GCAATCTTTGCAA | position (GCUCA-GGUGA); | 0.62% |
| | | TGATGGCAGCATT | 1/1 C/A mismatch at +17 position | ADAR1/2 specificity: |
| | | GGGATACAGTGT | | 0.46 |
| | | GAAGAGCAGCA | | |
| SEQ ID | LRRK2_ | CCCTGGTGTGCCC | 6/6 symmetric internal loop at −14 | ADAR1 on target: 0.28% |
| NO: 150 | bnPCR_ | TCTGATGTTTTTT | position (UGCAAA-AAACGU); | ADAR1 specificity: 0.77 |
| | ID22759_ | AGGGGATTCTAC | 1/1 U/G wobble base pair at −3 | ADAR2 on target: 0.68% |
| | count1590__ | AACAGTACGGTG | position; | ADAR2 specificity: 0.74 |
| | −14_26_ | AAGTGCCGTGGTC | 1/1 A/C mismatch at 0 position; | ADAR1/2 on target: |
| | gID_08476 | AGCAATCAAACG | 1/1 U/G wobble base pair at +4 | 0.66% |
| | | TATGATGGCAGC | position; | ADAR1/2 specificity: |
| | | ATTGGGATACAGT | 5/5 symmetric internal loop at +6 | 0.77 |
| | | GTGAAGAGCAGC | position (GCUCA-GGUGA); | |
| | | A | 1/1 C/A mismatch at +17 position; | |
| | | | 6/6 symmetric internal loop at +26 | |
| | | | position (GGGGAU-UAGGGG) | |
| SEQ ID | LRRK2_ | CCCTGGTGTGCCC | 9/9 symmetric internal loop at −19 | ADAR1 on target: 0.43% |
| NO: 151 | bnPCR_ | TCTGATGTTTTTC | position (GCCAUCAUU- | ADAR1 specificity: 0.74 |
| | ID22357_ | TAGATATTCTACG | CAUUAUUUG); | ADAR2 on target: 0.64% |
| | count844__ | GCAGTTCATAGCA | 1/1 C/A mismatch at −8 position; | ADAR2 specificity: 0.5 |
| | −22_26_ | ATCCCGTAGTCAA | 1/1 A/C mismatch at 0 position; | ADAR1/2 on target: |
| | gID_02673 | CAATCTTTGCCAT | 1/1 C/C mismatch at +2 position; | 0.52% |
| | | TATTTGAGCATTG | 2/2 symmetric bulge at +9 position | ADAR1/2 specificity: |
| | | GGATACAGTGTG | (CA-AU); | 0.57 |
| | | AAGAGCAGCA | 1/1 U/G mismatch at +12 position; | |
| | | | 1/1 U/G wobble base pair at +18 | |
| | | | position; | |
| | | | 1/1 G/U wobble base pair at +26 | |
| | | | position; | |
| | | | 5/5 symmetric internal loop at +27 | |
| | | | position (GGGAU-CUAGA) | |
| SEQ ID | LRRK2_ | CCCTGGTGTGCCC | 8/8 symmetric internal loop at −12 | ADAR1 on target: 0.4% |
| NO: 152 | bnPCR_ | TCTGATGTTTTTC | position (UGCAAAGA- | ADAR1 specificity: 0.82 |
| | ID22357_ | GGAGGATTCTAC | GGGUCCCC); | ADAR2 on target: 0.52% |
| | count844__ | GGCAGTTCATAGC | 1/1 C/A mismatch at −8 position; | ADAR2 specificity: 0.71 |
| | −12_26_ | AATCCCGTAGTCA | 1/1 A/C mismatch at 0 position; | ADAR1/2 on target: |
| | gID_07465 | ACAAGGGTCCCC | 1/1 C/C mismatch at +2 position; | 0.48% |
| | | ATGATGGCAGCA | 2/2 symmetric bulge at +9 position | ADAR1/2 specificity: |
| | | TTGGGATACAGTG | (CA-AU); | 0.74 |
| | | TGAAGAGCAGCA | 1/1 U/U mismatch at +12 position; | |
| | | | 1/1 U/G wobble base pair at +18 | |
| | | | position; | |
| | | | 6/6 symmetric internal loop at +26 | |
| | | | position (GGGGAU-CGGAGG) | |
| SEQ ID | LRRK2_ | CCCTGGTGTGCCC | 8/8 symmetric internal loop at −12 | ADAR1 on target: 0.3% |
| NO: 153 | bnPCR_ | TCTGATGTTTTTA | position (UGCAAAGA- | ADAR1 specificity: 0.79 |
| | ID22357_ | TCCCCATCACCCT | CAAUAUCC); | ADAR2 on target: 0.68% |
| | count844__ | GCAGTTCATAGCA | 1/1 C/A mismatch at −8 position; | ADAR2 specificity: 0.67 |
| | −12_18_ | ATCCCGTAGTCAA | 1/1 A/C mismatch at 0 position; | ADAR1/2 on target: |
| | gID_11904 | CAACAATATCCAT | 1/1 C/C mismatch at +2 position; | 0.65% |
| | | GATGGCAGCATT | 2/2 symmetric bulge at +9 position | ADAR1/2 specificity: |
| | | GGGATACAGTGT | (CA-AU); | 0.73 |
| | | GAAGAGCAGCA | 1/1 U/U mismatch at +12 position; | |
| | | | 6/6 symmetric internal loop at +18 | |
| | | | position (UGUAGA-CACCCU) | |
| SEQ ID | LRRK2_ | CCCTGGTGTGCCC | 6/6 symmetric internal loop at −22 | ADAR1 on target: 0.5% |
| NO: 154 | bnPCR_ | TCTGATGTTTTTC | position (GCCAUC-AAGCGA); | ADAR1 specificity: 0.66 |
| | ID16690_ | TGGTGATTCTACA | 1/1 C/A mismatch at −8 position; | ADAR2 on target: 0.64% |
| | count1976__ | ACAGTACTGAGCT | 10/10 symmetric internal loop at | ADAR2 specificity: 0.51 |
| | −22_26_ | ATCCCGAATTCAA | −4->5 position (CUACAGCAUU- | ADAR1/2 on target: |
| | gID_01893 | CAATCTTTGCAAT | UAUCCCGAAU); | 0.6% |
| | | AAGCGAAGCATT | 1/1 C/A mismatch at +17 position; | ADAR1/2 specificity: |
| | | GGGATACAGTGT | 1/1 G/G mismatch at +26 position; | 0.56 |
| | | GAAGAGCAGCA | 1/1 G/U wobble base pair at +27 | |
| | | | position; | |
| | | | 4/4 symmetric bulge at +28 | |
| | | | position (GGAU-CUGG) | |
| SEQ ID | LRRK2_ | CCCTGGTGTGCCC | 6/6 symmetric internal loop at −22 | ADAR1 on target: 0.22% |
| NO: 155 | bnPCR_ | TCTGATGTTTTTA | position (GCCAUC-CAAUCA); | ADAR1 specificity: 0.7 |
| | ID16690_ | TCCCCATTCGAAA | 1/1 C/A mismatch at −8 position; | ADAR2 on target: 0.61% |
| | count1976__ | AAAGTACTGAGC | 10/10 symmetrica internal loop at | ADAR2 specificity: 0.61 |
| | −22_16_ | TATCCCGAATTCA | −4->5 position (CUACAGCAUU- | ADAR1/2 on target: |
| | gID_06801 | ACAATCTTTGCAA | UAUCCCGAAU); | 0.57% |
| | | TCAATCAAGCATT | 6/6 symmetric internal loop at +16 | ADAR1/2 specificity: |
| | | GGGATACAGTGT | position (GCUGUA-GAAAAA) | 0.61 |
| | | GAAGAGCAGCA | | |
| SEQ ID | LRRK2_ | CCCTGGTGTGCCC | 6/6 symmetric internal loop at −16 | ADAR1 on target: 0.22% |
| NO: 156 | bnPCR_ | TCTGATGTTTTTA | position (AUUGCA-CUAUUA); | ADAR1 specificity: 0.8 |
| | ID16690_ | TCCCCATTCGAAT | 1/1 C/A mismatch at −8 position; | ADAR2 on target: 0.66% |
| | count1976__ | AAAGTACTGAGC | 10/10 symmetric internal loop at | ADAR2 specificity: 0.67 |
| | −16_16_ | TATCCCGAATTCA | −4->5 position (CUACAGCAUU- | ADAR1/2 on target: |
| | gID_10719 | ACAATCTTCTATT | UAUCCCGAAU); | 0.6% |
| | | AGATGGCAGCAT | 6/6 symmetric internal loop at +16 | ADAR1/2 specificity: 0.7 |
| | | TGGGATACAGTGT | position (GCUGUA-GAAUAA) | |
| | | GAAGAGCAGCA | | |
| SEQ ID | LRRK2_ | CCCTGGTGTGCCC | 1/1 C/U mismatch at −8 position; | ADAR1 on target: 0.49% |
| NO: 157 | bnPCR_ | TCTGATGTTTTTA | 1/1 U/G wobble base pair at −3 | ADAR1 specificity: 0.54 |
| | ID14524_ | TCCCCATTCTACA | position; | ADAR2 on target: 0.7% |
| | count2063__ | GCAGTAGTGTGC | 1/1 A/C mismatch at 0 position; | ADAR2 specificity: 0.38 |
| | NoLoops_ | AGTGCCGTGGTCA | 1/1 U/g wobble base pair at +4 | ADAR1/2 on target: |
| | gID_01159 | TCAATCTTTGCAA | position; | 0.66% |
| | | TGATGGCAGCATT | 1/1 U/U mismatch at +8 position; | ADAR1/2 specificity: |
| | | GGGATACAGTGT | 1/1 G/G mismatch at +11 position | 0.43 |
| | | GAAGAGCAGCA | | |
| SEQ ID | LRRK2_ | CCCTGGTGTGCCC | 6/6 symmetric internal loop at −14 | ADAR1 on target: 0.31% |
| NO: 158 | bnPCR_ | TCTGATGTTTTTT | position (UGCAAA-AAACGU); | ADAR1 specificity: 0.77 |
| | ID14524_ | AGGGGATTCTAC | 1/1 C/U mismatch at −8 position; | ADAR2 on target: 0.72% |
| | count2063__ | AGCAGTAGTGTG | 1/1 U/G wobble base pair at −3 | ADAR2 specificity: 0.69 |
| | −14_26_ | CAGTGCCGTGGTC | position; | ADAR1/2 on target: |
| | gID_05349 | ATCAATCAAACGT | 1/1 A/C mismatch at 0 position; | 0.68% |
| | | ATGATGGCAGCA | 1/1 U/G wobble base pair at +4 | ADAR1/2 specificity: |
| | | TTGGGATACAGTG | position; | 0.73 |
| | | TGAAGAGCAGCA | 1/1 U/U mismatch at +8 position; | |
| | | | 1/1 G/G mismatch at +11 position; | |
| | | | 6/6 symmetric internal loop at +26 | |
| | | | position (GGGGAU-UAGGGG) | |
| SEQ ID | LRRK2_ | CCCTGGTGTGCCC | 6/6 symmetric internal loop at −22 | ADAR1 on target: 0.37% |
| NO: 159 | bnPCR_ | TCTGATGTTTTTC | position (GCCAUC-AAGAGA); | ADAR1 specificity: 0.68 |
| | ID8030_ | AGTAGCTTCTACA | 1/1 U/G wobble base pair at −3 | ADAR2 on target: 0.6% |
| | count919__ | GCAGTTCGGAGG | position; | ADAR2 specificity: 0.61 |
| | −22_26_ | AATCCCGAGGTC | 1/1 A/A mismatch at −2 position; | ADAR1/2 on target: |
| | gID_12156 | AGCAATCTTTGCA | 1/1 A/C mismatch at 0 position; | 0.56% |
| | | ATAAGAGAAGCA | 1/1 C/C mismatch at +2 position; | ADAR1/2 specificity: |
| | | TTGGGATACAGTG | 1/1 G/G mismatch at +6 position; | 0.68 |
| | | TGAAGAGCAGCA | 3/3 symmetric bulge at +10 | |
| | | | position (AGU-UCG); | |
| | | | 7/7 symmetric internal loop at +25 | |
| | | | position (UGGGGAU- | |
| | | | CAGUAGC) | |
| SEQ ID | LRRK2_ | CCCTGGTGTGCCC | 6/6 symmetric internal loop at −20 | ADAR1 on target: 0.37% |
| NO: 160 | bnPCR_ | TCTGATGTTTTTT | position (CAUCAU-UACUAC); | ADAR1 specificity: 0.72 |
| | ID8030_ | AGGGGCTTCTACA | 1/1 U/G wobble base pair at −3 | ADAR2 on target: 0.59% |
| | count919__ | GCAGTTCGGAGG | position; | ADAR2 specificity: 0.67 |
| | −20_26_ | AATCCCGAGGTC | 1/1 A/A mismatch at −2 position; | ADAR1/2 on target: |
| | gID_00460 | AGCAATCTTTGCA | 1/1 A/C mismatch at 0 position; | 0.53% |
| | | TACTACGCAGCAT | 1/1 C/C mismatch at +2 position; | ADAR1/2 specificity: |
| | | TGGGATACAGTGT | 1/1 G/G mismatch at +6 position; | 0.72 |
| | | GAAGAGCAGCA | 3/3 symmetric bulge at +10 | |
| | | | position (AGU-UCG); | |
| | | | 7/7 symmetric internal loop at +25 | |
| | | | position (UGGGGAU- | |
| | | | UAGGGGC) | |
| SEQ ID | LRRK2_ | CCCTGGTGTGCCC | 6/6 symmetric internal loop at −18 | ADAR1 on target: 0.36% |
| NO: 161 | bnPCR_ | TCTGATGTTTTTA | position (UCAUUG-GUAGCU); | ADAR1 specificity: 0.77 |
| | ID8030_ | TGGAACGTCTACA | 1/1 U/G wobble base pair at −3 | ADAR2 on target: 0.63% |
| | count919__ | GCAGTTCGGAGG | position; | ADAR2 specificity: 0.67 |
| | −18_24 | AATCCCGAGGTC | 1/1 A/A mismatch at −2 position; | ADAR1/2 on target: |
| | gID_04981 | AGCAATCTTTGGT | 1/1 A/C mismatch at 0 position; | 0.57% |
| | | AGCTTGGCAGCAT | 1/1 C/C mismatch at +2 position; | ADAR1/2 specificity: 0.7 |
| | | TGGGATACAGTGT | 1/1 G/G mismatch at +6 position; | |
| | | GAAGAGCAGCA | 3/3 symmetric bulge at +10 | |
| | | | position (AGU-UCG); | |
| | | | 6/6 symmetric internal loop at +24 | |
| | | | position (AUGGGG-GGAACG) | |
| SEQ ID | LRRK2_ | CCCTGGTGTGCCC | 6/6 symmetric internal loop at −18 | ADAR1 on target: 0.33% |
| NO: 162 | bnPCR_ | TCTGATGTTTTTA | position (UCAUUG-GUCUUC); | ADAR1 specificity: 0.78 |
| | ID8030_ | TCCAGCGGGTAC | 1/1 U/G wobble base pair at −3 | ADAR2 on target: 0.62% |
| | count919__ | AGCAGTTCGGAG | position; | ADAR2 specificity: 0.69 |
| | −18_22_ | GAATCCCGAGGT | 1/1 A/A mismatch at −2 position; | ADAR1/2 on target: |
| | gID_06761 | CAGCAATCTTTGG | 1/1 A/C mismatch at 0 position; | 0.5% |
| | | TCTTCTGGCAGCA | 1/1 C/C mismatch at +2 position; | ADAR1/2 specificity: |
| | | TTGGGATACAGTG | 1/1 G/G mismatch at +6 position; | 0.73 |
| | | TGAAGAGCAGCA | 3/3 symmetric bulge at +10 | |
| | | | position (AGU-UCG); | |
| | | | 6/6 symmetric internal loop at +22 | |
| | | | position (GAAUGG-AGCGGG) | |
| SEQ ID | LRRK2_ | CCCTGGTGTGCCC | 6/6 symmetric internal loop at −16 | ADAR1 on target: 0.43% |
| NO: 163 | bnPCR_ | TCTGATGTTTCAA | position (AUUGCA-ACCCUG); | ADAR1 specificity: 0.75 |
| | ID8030_ | CGGCCCTTCTACA | 1/1 U/G wobble base pair at −3 | ADAR2 on target: 0.56% |
| | count919__ | GCAGTTCGGAGG | position; | ADAR2 specificity: 0.65 |
| | −16_28_ | AATCCCGAGGTC | 1/1 A/A mismatch at −2 position; | ADAR1/2 on target: |
| | gID_07038 | AGCAATCTTACCC | 1/1 A/C mismatch at 0 position; | 0.53% |
| | | TGGATGGCAGCA | 1/1 C/C mismatch at +2 position; | ADAR1/2 specificity: |
| | | TTGGGATACAGTG | 1/1 G/G mismatch at +6 position; | 0.66 |
| | | TGAAGAGCAGCA | 3/3 symmetric bulge at +10 | |
| | | | position (AGU-UCG); | |
| | | | 1/0 asymmetric bulge at +25 | |
| | | | position (U-); | |
| | | | 5/6 asymmetric internal loop at | |
| | | | +29 position (GAUAA- | |
| | | | CAACGG) | |
| SEQ ID | LRRK2_ | CCCTGGTGTGCCC | 6/6 symmetric internal loop at −16 | ADAR1 on target: 0.38% |
| NO: 164 | bnPCR_ | TCTGATGTTTTTA | position (AUUGCA-AAAUUA); | ADAR1 specificity: 0.81 |
| | ID8030_ | TTGAGCCTCTACA | 1/1 U/G wobble base pair at −3 | ADAR2 on target: 0.58% |
| | count919__ | GCAGTTCGGAGG | position; | ADAR2 specificity: 0.69 |
| | −16_24_ | AATCCCGAGGTC | 1/1 A/A mismatch at −2 position; | ADAR1/2 on target: |
| | gID_09086 | AGCAATCTTAAAT | 1/1 A/C mismatch at 0 position; | 0.52% |
| | | TAGATGGCAGCA | 1/1 C/C mismatch at +2 position; | ADAR1/2 specificity: |
| | | TTGGGATACAGTG | 1/1 G/G mismatch at +6 position; | 0.72 |
| | | TGAAGAGCAGCA | 3/3 symmetric bulge at +10 | |
| | | | position (AGU-UCG); | |
| | | | 5/5 symmetric internal loop at +24 | |
| | | | position (AUGGG-GAGCC); | |
| | | | 1/1 G/U wobble base pair at +29 | |
| | | | position | |
| SEQ ID | LRRK2_ | CCCTGGTGTGCCC | 6/6 symmetric internal loop at −14 | ADAR1 on target: 0.35% |
| NO: 165 | bnPCR_ | TCTGATGTTTTTT | position (UGCAAA-AAACGU); | ADAR1 specificity: 0.82 |
| | ID8030_ | AGGGGCTTCTACA | 1/1 U/G wobble base pair at −3 | ADAR2 on target: 0.59% |
| | count919__ | GCAGTTCGGAGG | position; | ADAR2 specificity: 0.73 |
| | −14_26_ | AATCCCGAGGTC | 1/1 A/A mismatch at −2 position; | ADAR1/2 on target: |
| | gID_07899 | AGCAATCAAACG | 1/1 A/C mismatch at 0 position; | 0.55% |
| | | TATGATGGCAGC | 1/1 C/C mismatch at +2 position; | ADAR1/2 specificity: |
| | | ATTGGGATACAGT | 1/1 G/G mismatch at +6 position; | 0.75 |
| | | GTGAAGAGCAGC | 3/3 symmetric bulge at +10 | |
| | | A | position (AGU-UCG); | |
| | | | 7/7 symmetric internal loop at +25 | |
| | | | position (UGGGGAU- | |
| | | | UAGGGGC) | |
| SEQ ID | LRRK2_ | CCCTGGTGTGCCC | 6/6 symmetric internal loop at −12 | ADAR1 on target: 0.46% |
| NO: 166 | bnPCR_ | TCTGATGTTTTTA | position (CAAAGA-GACUAA); | ADAR1 specificity: 0.83 |
| | ID8030_ | TGAGGCGTCTACA | 1/1 U/G wobble base pair at −3 | ADAR2 on target: 0.67% |
| | count919__ | GCAGTTCGGAGG | position; | ADAR2 specificity: 0.7 |
| | −12_24_ | AATCCCGAGGTC | 1/1 A/A mismatch at −2 position; | ADAR1/2 on target: |
| | gID_03363 | AGCAAGACTAAC | 1/1 A/C mismatch at 0 position; | 0.58% |
| | | AATGATGGCAGC | 1/1 C/C mismatch at +2 position; | ADAR1/2 specificity: |
| | | ATTGGGATACAGT | 1/1 G/G mismatch at +6 position; | 0.76 |
| | | GTGAAGAGCAGC | 3/3 symmetric bulge at +10 | |
| | | A | position (AGU-UCG); | |
| | | | 6/6 symmetric internal loop at +24 | |
| | | | position (AUGGGG-GAGGCG) | |
| SEQ ID | LRRK2_ | CCCTGGTGTGCCC | 6/6 symmetric internal loop at −10 | ADAR1 on target: 0.5% |
| NO: 167 | bnPCR_ | TCTGATGTTTTTA | position (AAGAUU-UUAGCC); | ADAR1 specificity: 0.84 |
| | ID8030_ | TTGAGCCTCTACA | 1/1 U/G wobble base pair at −3 | ADAR2 on target: 0.48% |
| | count919__ | GCAGTTCGGAGG | position; | ADAR2 specificity: 0.75 |
| | −10_24_ | AATCCCGAGGTC | 1/1 A/A mismatch at −2 position; | ADAR1/2 on target: |
| | gID_09672 | AGCTTAGCCTGCA | 1/1 A/C mismatch at 0 position; | 0.59% |
| | | ATGATGGCAGCA | 1/1 C/C mismatch at +2 position; | ADAR1/2 specificity: |
| | | TTGGGATACAGTG | 1/1 G/G mismatch at +6 position; | 0.77 |
| | | TGAAGAGCAGCA | 3/3 symmetric bulge (AGU-UCG); | |
| | | | 5/5 symmetric internal loop at +24 | |
| | | | position (AUGGG-GAGCC); | |
| | | | 1/1 G/U wobble base pair at +29 | |
| | | | position | |
| SEQ ID | LRRK2_ | CCCTGGTGTGCCC | −20_6-6_internal_loop- | |
| NO: | bnPCR_ | TCTGATGTTTTTTA | symmetric_CAUCAU-UACUAC, | |
| 3081 | ID79791_ | GGGGATTCTACAT | −4_1-1_mismatch_C-C, −3_1- | |
| | count610__ | CTGTAGTGAGCAA | 1_wobble_U-G, 0_1- | |
| | −20_26 | TTCCGTGCTCAGC | 1_mismatch_A-C, 2_1- | |
| | | AATCTTTGCATAC | 1_mismatch_C-U, 11_1- | |
| | | TACGCAGCATTGG | 1_mismatch_G-G, 15_1- | |
| | | GATACAGTGTGAA | 1_mismatch_U-U, 17_1- | |
| | | AAGCAGCA | 1_mismatch_C-U, 26_6- | |
| | | | 6_internal_loop- | |
| | | | symmetric_GGGGAU-UAGGGG | |
| SEQ ID | LRRK2_ | CCCTGGTGTGCCC | −20_6-6_internal_loop- | |
| NO: | bnPCR_ | TCTGATGTTTTTTA | symmetric_CAUCAU-UACUAC, | |
| 3082 | ID16690_ | GGGGATTCTACAA | −8_1-1_mismatch_C-A, −4->5_10- | |
| | count1976__ | CAGTACTGAGCTA | 10_internal_loop- | |
| | −20_26 | TCCCGAATTCAAC | symmetric_CUACAGCAUU- | |
| | | AATCTTTGCATAC | UAUCCCGAAU, 17_1- | |
| | | TACGCAGCATTGG | 1_mismatch_C-A, 26_6- | |
| | | GATACAGTGTGAA | 6_internal_loop- | |
| | | AAGCAGCA | symmetric_GGGGAU-UAGGGG | |

### Example 15

### Machine Learning for Barbell Position

This example describes using machine learning to determine the positioning of barbells in a guide RNA that impact on-target editing and specificity score for editing a LRRK2 mRNA via ADAR. A set of 1709 engineered guide RNAs was used to train and test a random forest (RF) model. Of this set of guides, 1000 engineered guides were used to train the RF model and 709 engineered guides were used to test the accuracy of the trained RF model for predicting on-target editing and specificity score based on an engineered guide sequence. There was a high correlation between the predicted on-target editing and specificity score and the experimentally tested on-target editing and specificity score, indicating that the trained RF model accurately predicts on-target editing and specificity score based on an engineered guide sequence. This trained RF model was then used to determine the importance of characteristic features in the guide RNAs that impact on-target editing and specificity score, such as length of time for editing (20 sec, 1 min, 3 min, 10 min, 30 min, or 60 min), the ADAR used for editing (ADAR1, ADAR2, or ADAR1 and ADAR2), positioning of a right barbell (relative to the target adenosine to be edited), positioning of left barbell (relative to the target nucleotide to be edited). Right barbell positioning was identified as the most important feature for predicting the specificity of an engineered guide RNA and the third most important feature for predicting on-target editing. For engineered guide RNAs using ADAR1 for editing, the best positioning of the right barbell in an engineered guide RNA to achieve a high specificity score was +28 or +30 nts, wherein the positioning is relative to the target adenosine in the LRRK2 mRNA to be edited. For engineered guide RNAs using ADAR2 for editing, the best positioning of the right barbell in an engineered guide RNA to achieve a high specificity score was +24 or +26 nts, wherein the positioning is relative to the target adenosine in the LRRK2 mRNA to be edited.

### Example 16

Engineered Guide RNAs with Barbells Targeting DUX4

This example describes engineered guide RNAs with barbells targeting the polyadenylation (polyA) signal site (ATTAAA) in the “pLAM” region of DUX4 mRNA. One or more of the three terminal As in the polyA signal site sequence (ATTAAA) was targeted for editing via ADAR using the engineered guide RNA sequences of TABLE 4. Self-annealing RNA structures, which comprised (i) the engineered guide RNAs having barbells shown in TABLE 4 and (ii) the RNA sequences of the DUX4 region targeted by the engineered guide RNAs, were contacted with an RNA editing entity (e.g., a recombinant ADAR1 and/or ADAR2) for 30 minutes under conditions that allowed for editing. The regions targeted by the engineered guide RNAs were subsequently assessed for editing using next generation sequencing (NGS). Engineered guide RNAs having barbells that displayed favorable on-target editing of DUX4 for ADAR1 and/or ADAR2 are shown in TABLE 4. All polynucleotide sequences encoding for the engineered guide RNAs of TABLE 4, are also encompassed herein. Further, each engineered guide RNA sequence can be represented as a DNA sequence in which each U is replaced with a T. For each sequence, the structural features formed in the double stranded RNA substrate upon hybridization of the guide RNA to the target DUX4 RNA, are shown in the second column of TABLE 4 and the position of the left bell (“LB”) and right bell (“RB”) of the barbell are shown in the third and fourth columns, respectively. For reference, each structural feature formed within a guide-target RNA scaffold (target RNA sequence hybridized to an engineered guide RNA) is annotated as follows:
a. the position of the structural feature with respect to the target A (position 0) of the target RNA sequence, with a negative value indicating upstream (5′) of the target A and a positive value indicating downstream (3′) of the target A;
b. the number of bases in the target RNA sequence and the number of bases in the engineered guide RNA that together form the structural feature—for example, 6/6 indicates that six contiguous bases from the target RNA sequence and six contiguous bases from the engineered guide RNA form the structural feature;
c. the name of the structural feature (e.g., symmetric bulge, symmetric internal loop, asymmetric bulge, asymmetric internal loop, mismatch, or wobble base pair), and
d. the sequences of bases on the target RNA side and the engineered guide RNA side that participate in forming the structural feature.

For example, in SEQ ID NO: 172, “−12_6-6_internal_loop-symmetric_GAUUAG-AAACCG” is read as a structural feature formed in a guide-target RNA scaffold (target RNA sequence hybridized to an engineered guide RNA of SEQ ID NO: 172), where
a. the structural feature starts 12 nucleotides upstream (5′) (the −12 position) from the target A (0 position) of the target RNA sequence
b. six contiguous bases from the target RNA sequence and six contiguous bases from the engineered guide RNA form the structural feature
c. the structural feature is an internal symmetric loop
d. a sequence of GAUUAG from the target RNA side and a sequence of AAACCG from the engineered guide RNA side participate in forming the internal symmetric loop.

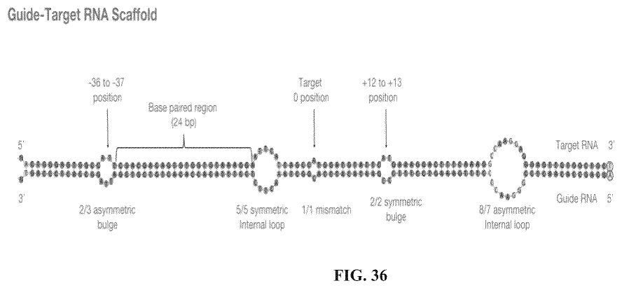

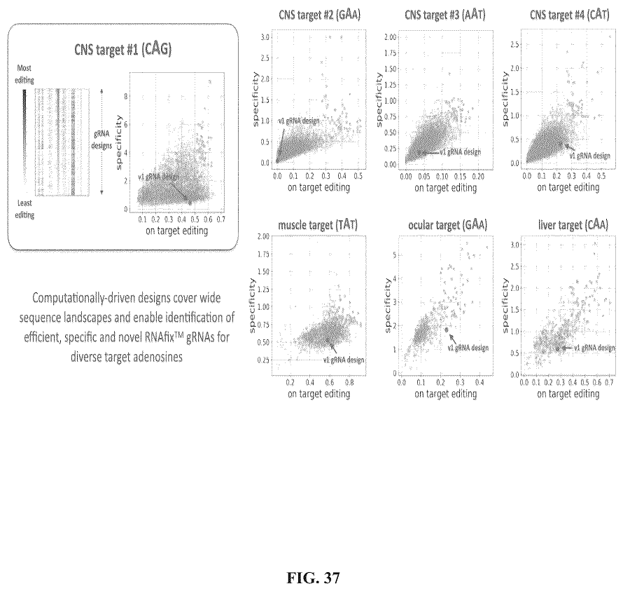

For reference, FIG. 36 can be used as an aid to visualize the structural features and the nomenclature disclosed herein. Each of the engineered guide RNAs of TABLE 4 showed editing of one or more of the three terminal As in the polyA signal site sequence (ATTAAA) of DUX4 mRNA, as summarized in TABLE 5, with the first A indicated as “P0” for position 0, the second A indicated as “P3” for position 3, the third A indicated as “P4” for position 4, the fourth A indicated as “P5” for position 5, and any of the As is indicated as “Any”. In TABLE 5, “A1” indicates ADAR1, “A2” indicates ADAR2, and “A1+2” indicates ADAR1 and ADAR 2. “Percent on-target editing is calculated by the following formula: the number of reads containing “G” at the target/the total number of reads.

Lengthy table referenced here
US20250009904A1-20250109-T00001
Please refer to the end of the specification for access instructions.

Lengthy table referenced here
US20250009904A1-20250109-T00002
Please refer to the end of the specification for access instructions.

### Example 17

Engineered Guide RNA with Barbells Targeting the MAPT TIS

This example describes sequences of engineered guide RNAs with barbells targeting the TIS of MAPT. Self-annealing RNA structures, which comprised (i) the engineered guide RNAs having barbells shown in TABLE 6 and (ii) the RNA sequences of the MAPT region targeted by the engineered guide RNAs, were contacted with an RNA editing entity (e.g., a recombinant ADAR1 and/or ADAR2) for 30 minutes under conditions that allowed for editing. The regions targeted by the engineered guide RNAs were subsequently assessed for editing by next generation sequencing (NGS). Engineered guide RNAs having barbells that displayed greater than 50% on-target editing of the MAPT TIS for ADAR1 and/or ADAR2, as quantified at a read depth of >200, are shown in TABLE 6. All polynucleotide sequences encoding for the engineered guide RNAs of TABLE 6, are also encompassed herein. Further, each engineered guide RNA sequence can be represented as a DNA sequence in which each U is replaced with a T. For each sequence, the structural features formed in the double stranded RNA substrate upon hybridization of the guide RNA to the target MAPT RNA, are shown in the second column of TABLE 6 and the position of the left bell (“LB”) and the right bell (“RB”) of the barbell are shown in the third and fourth columns, respectively. For reference, each structural feature is annotated as follows: position of the structural feature with a negative value indicating upstream of the target A and a positive value indicating downstream of the target A, the name of the structural feature (e.g., symmetric bulge, symmetric internal loop, asymmetric bulge, asymmetric internal loop, or mismatch), and the sequence of participating bases on the target RNA side and the guide RNA side. For example, in SEQ ID NO: 1519, “−22_3-3_bulge-symmetric_CUA-GAU” is read as a 3/3 symmetric bulge (CUA on the target side, GAU on the engineered guide RNA side) in the −22 position. For reference, FIG. 36 can be used as an aid to visualize the structural features and the nomenclature disclosed herein. Table 6 further includes the amount of on target editing achieved via ADAR1 or ADAR2 seperately, as well as ADAR1 and ADAR2. The specificity of each guide was also calculated for each engineered guide via ADAR1, ADAR2, and ADAR1+ADAR2. Specificity as provided in Table 6 was calculated using the formula: Specificity=(fraction on-target editing+1)/(sum(non-synonymous off-target editing)). These data highlight the diverse sequence space represented by the MAPT-targeting engineered guide RNAs of the present disclosure, which have a range of different structural features that drive sequence diversity and which exhibit high on-target editing efficiency.

Lengthy table referenced here
US20250009904A1-20250109-T00003
Please refer to the end of the specification for access instructions.

### Example 18

Engineered Guide RNAs with Barbells Targeting the SNCA TIS

This example describes sequences of engineered guide RNAs with barbells targeting the TIS of SNCA. Self-annealing RNA structures, which comprised (i) the engineered guide RNAs having barbells shown in TABLE 7 and (ii) the RNA sequences of the SNCA region targeted by the engineered guide RNAs, were contacted with an RNA editing entity (e.g., a recombinant ADAR1 and/or ADAR2) for 30 minutes under conditions that allowed for editing. The regions targeted by the engineered guide RNAs were subsequently assessed for editing by next generation sequencing (NGS). Engineered guide RNAs having barbells that displayed greater than 50% on-target editing of the SNCA TIS for ADAR1 and/or ADAR2, as quantified at a read depth of >200, are shown in TABLE 7. All polynucleotide sequences encoding for the engineered guide RNA of TABLE 7 are also encompassed herein. Further, each engineered guide RNA sequence can be represented as a DNA sequence in which each U is replaced with a T. For each sequence, the structural features formed in the double stranded RNA substrate upon hybridization of the guide RNA to the target SNCA RNA, are shown in the second column of TABLE 7 and the position of the left bell (“LB”) and the right bell (“RB”) of the barbell are shown in the third and fourth columns, respectively. For reference, each structural feature is annotated as follows: position of the structural feature with a negative value indicating upstream of the target A and a positive value indicating downstream of the target A, the name of the structural feature (e.g., symmetric bulge, symmetric internal loop, asymmetric bulge, asymmetric internal loop, or mismatch), and the sequence of participating bases on the target RNA side and the guide RNA side. For example, in SEQ ID NO: 2480, “−33_4-4_bulge-symmetric_UUCG-ACAU” is read as a 4/4 symmetric bulge (UUCG on the target side, ACAU on the engineered guide RNA side) in the −33 position. For reference, FIG. 36 can be used as an aid to visualize the structural features and the nomenclature disclosed herein. TABLE 7 further includes the amount of on target editing achieved via ADAR1 or ADAR2 seperately, as well as ADAR1 and ADAR2. The specificity of each guide was also calculated for each engineered guide via ADAR1, ADAR2, and ADAR1+ADAR2. Specificity as provided in Table 7 was calculated using the formula: Specificity=(fraction on-target editing+1)/(sum(non-synonymous off-target editing)). These data highlight the diverse sequence space represented by the SNCA TIS-targeting engineered guide RNAs of the present disclosure, which have a range of different structural features that drive sequence diversity and which exhibit high on-target editing efficiency.

TABLE 7
Engineered Guide RNAs with Barbells Targeting the SNCA TIS

| SEQ | | | | | ADAR1 | ADAR2 | ADAR1/2 | | | |
|---|---|---|---|---|---|---|---|---|---|---|
| ID | | | | | on- | on- | on- | ADAR1 | ADAR2 | ADAR1/2 |
| NO | Structural Features (target/guide) | Engineered Guide RNA Sequence | LB | RB | target | target | target | specificity | specificity | specificity |
| 2480 | −33_4-4_bulge-symmetric_UUCG- | GCCACAUGAGGGUCCUUGG | −10 | 38 | 0.501 | 0.91 | 0.908 | 0.739 | 0.641 | 0.7 |
| | ACAU | CCUUUGAAAGUCCUUUCAU | | | | | | | | |
| | −20_4-4_bulge-symmetric_UGGU- | GAAUACAGCCACGGCUAAU | | | | | | | | |
| | UAGU | UGACAUGGCUUACUAGUCA | | | | | | | | |
| | −10_6-6_internal_loop- | CUGUCGUACAUUGGCCACU | | | | | | | | |
| | symmetric_AGGAAU-CAUGGC | CCCAGU | | | | | | | | |
| | −6_0-1_bulge-asymmetric_-U | | | | | | | | | |
| | 0_1-1_mismatch_A-C | | | | | | | | | |
| | 4_1-1_mismatch_A-G | | | | | | | | | |
| | 38_6-6_internal_loop- | | | | | | | | | |
| | symmetric_GGGAGU-UGAGGG | | | | | | | | | |
| 2481 | −33_4-4_bulge-symmetric_UUCG- | GCCACAUGGAGGUCCUUGG | −4 | 38 | 0.506 | 0.541 | 0.725 | 0.862 | 0.579 | 0.673 |
| | ACAU | CCUUUGAAAGUCCUUUCAU | | | | | | | | |
| | −20_4-4_bulge-symmetric_UGGU- | GAAUACAACCACGGCGUCG | | | | | | | | |
| | UAGU | CUAUUCCUUUACUAGUCAC | | | | | | | | |
| | −4_6-6_internal_loop- | UGUCGUACAUUGGCCACUC | | | | | | | | |
| | symmetric_UCAUUA-GUCGCU | CCAGU | | | | | | | | |
| | 0_1-1_mismatch_A-C | | | | | | | | | |
| | 4_1-1_mismatch_A-A | | | | | | | | | |
| | 38_6-6_internal_loop- | | | | | | | | | |
| | symmetric_GGGAGU-UGGAGG | | | | | | | | | |
| 2482 | −33_4-4_bulge-symmetric_UUCG- | GCCACACAGACCUCCUCUAC | −12 | 28 | 0.115 | 0.747 | 0.735 | 1.02 | 1.554 | 1.482 |
| | ACAU | GGUUGAAAGUCCUUUCAUG | | | | | | | | |
| | −20_4-4_bulge-symmetric_UGGU- | AAUACUCUCCACGGCAAUG | | | | | | | | |
| | UAGU | AAUGAAUUGACUAGUCACU | | | | | | | | |
| | −12_6-6_internal_loop- | GUCGUACAUUGGCCACUCCC | | | | | | | | |
| | symmetric_AAAGGA-GAAUUG | AGU | | | | | | | | |
| | −4_1-0_bulge-asymmetric_A- | | | | | | | | | |
| | 0_1-1_mismatch_A-C | | | | | | | | | |
| | 5_1-2_bulge-asymmetric_U-UC | | | | | | | | | |
| | 28_6-6_internal_loop- | | | | | | | | | |
| | symmetric_AGGCCA-CUACGG | | | | | | | | | |
| | 40_4-4_bulge-symmetric_GAGU- | | | | | | | | | |
| | CAGA | | | | | | | | | |
| 2483 | −33_4-4_bulge-symmetric_UUCG- | GCCACACAGACCUCCUUGGC | −8 | 24 | 0.087 | 0.62 | 0.661 | 0.982 | 1.414 | 1.433 |
| | ACAU | AAAGCUAAGUCCUUUCAUG | | | | | | | | |
| | −20_4-4_bulge-symmetric_UGGU- | AAUAAUUCCACGGCUGAUC | | | | | | | | |
| | UAGU | CGGGGCUUUACUAGUCACU | | | | | | | | |
| | −8_6-6_internal_loop- | GUCGUACAUUGGCCACUCCC | | | | | | | | |
| | symmetric_GAAUUC-CCGGGG | AGU | | | | | | | | |
| | −5_1-1_wobble_U-G | | | | | | | | | |
| | 0_1-1_mismatch_A-C | | | | | | | | | |
| | 5_2-2_bulge-symmetric_UG-AU | | | | | | | | | |
| | 24_6-6_internal_loop- | | | | | | | | | |
| | symmetric_UCAAAG-AAAGCU | | | | | | | | | |
| | 40_4-4_bulge-symmetric_GAGU- | | | | | | | | | |
| | CAGA | | | | | | | | | |
| 2484 | −33_4-4_bulge-symmetric_UUCG- | GCCACACAGACCUCGAUCUA | −12 | 30 | 0.177 | 0.758 | 0.803 | 1.047 | 1.287 | 1.412 |
| | ACAU | CUUUGAAAGUCCUUUCAUG | | | | | | | | |
| | −20_4-4_bulge-symmetric_UGGU- | GCUACAUCCACGGCUCAAG | | | | | | | | |
| | UAGU | AAUGUACAGACUAGUCACU | | | | | | | | |
| | −12_6-6_internal_loop- | GUCGUACAUUGGCCACUCCC | | | | | | | | |
| | symmetric_AAAGGA-GUACAG | AGU | | | | | | | | |
| | −5_3-3_bulge-symmetric_AUU-CAA | | | | | | | | | |
| | 0_1-1_mismatch_A-C | | | | | | | | | |
| | 9_1-1_mismatch_U-C | | | | | | | | | |
| | 10_1-1_wobble_U-G | | | | | | | | | |
| | 30_6-6_internal_loop- | | | | | | | | | |
| | symmetric_GCCAAG-GAUCUA | | | | | | | | | |
| | 40_4-4_bulge-symmetric_GAGU- | | | | | | | | | |
| | CAGA | | | | | | | | | |
| 2485 | −33_4-4_bulge-symmetric_UUCG- | GCCACACAGACCUCCUAAAG | −10 | 28 | 0.193 | 0.739 | 0.732 | 1.052 | 1.464 | 1.42 |
| | ACAU | AGUUGAAAGUCCUUUCAUG | | | | | | | | |
| | −20_4-4_bulge-symmetric_UGGU- | UAUACAUCCACGACUCCUG | | | | | | | | |
| | UAGU | ACCAGUGUUACUAGUCACU | | | | | | | | |
| | −10_6-6_internal_loop- | GUCGUACAUUGGCCACUCCC | | | | | | | | |
| | symmetric_AGGAAU-CCAGUG | AGU | | | | | | | | |
| | −5_2-2_bulge-symmetric_UU-CC | | | | | | | | | |
| | −2_1-1_mismatch_C-A | | | | | | | | | |
| | 0_1-1_mismatch_A-C | | | | | | | | | |
| | 10_1-1_mismatch_U-U | | | | | | | | | |
| | 28_6-6_internal_loop- | | | | | | | | | |
| | symmetric_AGGCCA-AAAGAG | | | | | | | | | |
| | 40_4-4_bulge-symmetric_GAGU- | | | | | | | | | |
| | CAGA | | | | | | | | | |
| 2486 | −33_4-4_bulge-symmetric_UUCG- | GCCACACAGACCUCCUUGUU | −8 | 26 | 0.027 | 0.613 | 0.652 | 0.958 | 1.446 | 1.453 |
| | ACAU | AAGGGAAAGUCCUUUCCCG | | | | | | | | |
| | −20_4-4_bulge-symmetric_UGGU- | AAUACAUCCACGGCUAAUU | | | | | | | | |
| | UAGU | CUCGACUUUACUAGUCACU | | | | | | | | |
| | −8_6-6_internal_loop- | GUCGUACAUUGGCCACUCCC | | | | | | | | |
| | symmetric_GAAUUC-UCUCGA | AGU | | | | | | | | |
| | 0_1-1_mismatch_A-C | | | | | | | | | |
| | 12_2-2_bulge-symmetric_AU-CC | | | | | | | | | |
| | 26_6-6_internal_loop- | | | | | | | | | |
| | symmetric_AAAGGC-UUAAGG | | | | | | | | | |
| | 40_4-4_bulge-symmetric_GAGU- | | | | | | | | | |
| | CAGA | | | | | | | | | |
| 2487 | −33_4-4_bulge-symmetric_UUCG- | GCCACACAGACCAGGAUCGC | −6 | 32 | 0.114 | 0.928 | 0.923 | 0.951 | 1.498 | 1.475 |
| | ACAU | CUUUGAAAGUCCUUCCCUG | | | | | | | | |
| | −20_4-4_bulge-symmetric_UGGU- | AAUACAUCCACGGCUACGA | | | | | | | | |
| | UAGU | CAGUCCUUUACUAGUCACU | | | | | | | | |
| | −6_6-6_internal_loop- | GUCGUACAUUGGCCACUCCC | | | | | | | | |
| | symmetric_AUUCAU-CGACAG | AGU | | | | | | | | |
| | 0_1-1_mismatch_A-C | | | | | | | | | |
| | 13_1-1_mismatch_U-C | | | | | | | | | |
| | 15_1-1_mismatch_A-C | | | | | | | | | |
| | 32_6-6_internal_loop- | | | | | | | | | |
| | symmetric_CAAGGA-AGGAUC | | | | | | | | | |
| | 40_4-4_bulge-symmetric_GAGU- | | | | | | | | | |
| | CAGA | | | | | | | | | |
| 2488 | −33_4-4_bulge-symmetric_UUCG- | GCCACACAGACCUCAUUCGA | −4 | 30 | 0.064 | 0.704 | 0.79 | 0.951 | 1.449 | 1.527 |
| | ACAU | CUUUGAAAGUCCUUUCAGG | | | | | | | | |
| | −20_4-4_bulge-symmetric_UGGU- | AAUAACUCCACGGCACGGU | | | | | | | | |
| | UAGU | CAUUCCUUUACUAGUCACU | | | | | | | | |
| | −4_6-6_internal_loop- | GUCGUACAUUGGCCACUCCC | | | | | | | | |
| | symmetric_UCAUUA-ACGGUC | AGU | | | | | | | | |
| | 0_1-1_mismatch_A-C | | | | | | | | | |
| | 5_2-2_bulge-symmetric_UG-AC | | | | | | | | | |
| | 12_1-1_mismatch_A-G | | | | | | | | | |
| | 30_6-6_internal_loop- | | | | | | | | | |
| | symmetric_GCCAAG-AUUCGA | | | | | | | | | |
| | 40_4-4_bulge-symmetric_GAGU- | | | | | | | | | |
| | CAGA | | | | | | | | | |
| 2489 | −33_4-4_bulge-symmetric_UUCG- | GCCACACAGACCUCCUUGAU | −6 | 26 | 0.026 | 0.94 | 0.932 | 0.936 | 1.755 | 1.707 |
| | ACAU | AGCAGAAAGUCAAUUUAUG | | | | | | | | |
| | −20_4-4_bulge-symmetric_UGGU- | AAUACAUCCACGGCUAUUU | | | | | | | | |
| | UAGU | AUGUCCUUUACUAGUCACU | | | | | | | | |
| | −6_6-6_internal_loop- | GUCGUACAUUGGCCACUCCC | | | | | | | | |
| | symmetric_AUUCAU-UUUAUG | AGU | | | | | | | | |
| | 0_1-1_mismatch_A-C | | | | | | | | | |
| | 14_1-1_wobble_G-U | | | | | | | | | |
| | 17_2-2_bulge-symmetric_AG-AA | | | | | | | | | |
| | 26_6-6_internal_loop- | | | | | | | | | |
| | symmetric_AAAGGC-AUAGCA | | | | | | | | | |
| | 40_4-4_bulge-symmetric_GAGU- | | | | | | | | | |
| | CAGA | | | | | | | | | |
| 2490 | −33_4-4_bulge-symmetric_UUCG- | GCCACACAGACCUCCUCUUU | −6 | 28 | 0.025 | 0.853 | 0.867 | 0.931 | 1.63 | 1.605 |
| | ACAU | UGUUGAAAGUAGGUUCAUG | | | | | | | | |
| | −20_4-4_bulge-symmetric_UGGU- | ACACAUCCACGGCUACGUU | | | | | | | | |
| | UAGU | UCUCCUUUACUAGUCACUG | | | | | | | | |
| | −6_6-6_internal_loop- | UCGUACAUUGGCCACUCCCA | | | | | | | | |
| | symmetric_AUUCAU-CGUUUC | GU | | | | | | | | |
| | 0_1-1_mismatch_A-C | | | | | | | | | |
| | 8_2-1_bulge-asymmetric_AU-C | | | | | | | | | |
| | 17_3-3_bulge-symmetric_AGG-AGG | | | | | | | | | |
| | 28_6-6_internal_loop- | | | | | | | | | |
| | symmetric_AGGCCA-CUUUUG | | | | | | | | | |
| | 40_4-4_bulge-symmetric_GAGU- | | | | | | | | | |
| | CAGA | | | | | | | | | |
| 2491 | −33_4-4_bulge-symmetric_UUCG- | GCCACACAGACCUCCUAUAU | −6 | 28 | 0.036 | 0.832 | 0.843 | 0.938 | 1.523 | 1.544 |
| | ACAU | UCUUGAAAGUCCUUGCAUG | | | | | | | | |
| | −20_4-4_bulge-symmetric_UGGU- | AAUGCAUUCACGGCUAUCA | | | | | | | | |
| | UAGU | GGCUCCUUUACUAGUCACU | | | | | | | | |
| | −6_6-6_internal_loop- | GUCGUACAUUGGCCACUCCC | | | | | | | | |
| | symmetric_AUUCAU-UCAGGC | AGU | | | | | | | | |
| | 0_1-1_mismatch_A-C | | | | | | | | | |
| | 3_1-1_wobble_G-U | | | | | | | | | |
| | 7_1-1_wobble_U-G | | | | | | | | | |
| | 15_1-1_mismatch_A-G | | | | | | | | | |
| | 28_6-6_internal_loop- | | | | | | | | | |
| | symmetric_AGGCCA-AUAUUC | | | | | | | | | |
| | 40_4-4_bulge-symmetric_GAGU- | | | | | | | | | |
| | CAGA | | | | | | | | | |
| 2492 | −33_4-4_bulge-symmetric_UUCG- | GCCACACAGACCUCCUUGUA | −10 | 26 | 0.153 | 0.689 | 0.692 | 1.027 | 1.486 | 1.42 |
| | ACAU | UGACGAAAGUCCUUUCAUG | | | | | | | | |
| | −20_4-4_bulge-symmetric_UGGU- | AAUACAAUCCACGGCUAAU | | | | | | | | |
| | UAGU | GACGCGUAUUACUAGUCAC | | | | | | | | |
| | −10_6-6_internal_loop- | UGUCGUACAUUGGCCACUC | | | | | | | | |
| | symmetric_AGGAAU-CGCGUA | CCAGU | | | | | | | | |
| | 0_1-1_mismatch_A-C | | | | | | | | | |
| | 5_0-1_bulge-asymmetric_-A | | | | | | | | | |
| | 26_6-6_internal_loop- | | | | | | | | | |
| | symmetric_AAAGGC-UAUGAC | | | | | | | | | |
| | 40_4-4_bulge-symmetric_GAGU- | | | | | | | | | |
| | CAGA | | | | | | | | | |
| 2493 | −33_4-4_bulge-symmetric_UUCG- | GCCACACAGACCUCCUAAUA | −10 | 28 | 0.07 | 0.932 | 0.9 | 0.955 | 1.66 | 1.583 |
| | ACAU | AGUUGAAAGUCUUUUCAUG | | | | | | | | |
| | −20_4-4_bulge-symmetric_UGGU- | AAUACAUUCACGGCUAAUG | | | | | | | | |
| | UAGU | ACGCGAAUUACUAGUCACU | | | | | | | | |
| | −10_6-6_internal_loop- | GUCGUACAUUGGCCACUCCC | | | | | | | | |
| | symmetric_AGGAAU-CGCGAA | AGU | | | | | | | | |
| | 0_1-1_mismatch_A-C | | | | | | | | | |
| | 3_1-1_wobble_G-U | | | | | | | | | |
| | 18_1-1_wobble_G-U | | | | | | | | | |
| | 28_6-6_internal_loop- | | | | | | | | | |
| | symmetric_AGGCCA-AAUAAG | | | | | | | | | |
| | 40_4-4_bulge-symmetric_GAGU- | | | | | | | | | |
| | CAGA | | | | | | | | | |
| 2494 | −33_4-4_bulge-symmetric_UUCG- | GCCACACAGACCAAAGAUG | −20 | 32 | 0.086 | 0.919 | 0.921 | 0.812 | 1.583 | 1.529 |
| | ACAU | CCUUUGAAAGUCCUUUCAG | | | | | | | | |
| | −20_6-6_internal_loop- | GAAUACAUCCACGGCUAAU | | | | | | | | |
| | symmetric_UGUGGU-UGAGGU | GAAUUCCUUUACUGAGGUC | | | | | | | | |
| | 0_1-1_mismatch_A-C | UGUCGUACAUUGGCCACUC | | | | | | | | |
| | 12_1-1_mismatch_A-G | CCAGU | | | | | | | | |
| | 32_6-6_internal_loop- | | | | | | | | | |
| | symmetric_CAAGGA-AAAGAU | | | | | | | | | |
| | 40_4-4_bulge-symmetric_GAGU- | | | | | | | | | |
| | CAGA | | | | | | | | | |
| 2495 | −33_4-4_bulge-symmetric_UUCG- | GCCACAUAGGGAUCCUUGG | −8 | 38 | 0.105 | 0.694 | 0.724 | 0.622 | 0.977 | 0.898 |
| | ACAU | CCUUUGAAAGUCCUUUCAG | | | | | | | | |
| | −20_4-4_bulge-symmetric_UGGU- | GAAUACAUCCACGGCUAAU | | | | | | | | |
| | UAGU | CUGCGGCUUUACUAGUCAC | | | | | | | | |
| | −8_6-6_internal_loop- | UGUCGUACAUUGGCCACUC | | | | | | | | |
| | symmetric_GAAUUC-CUGCGG | CCAGU | | | | | | | | |
| | 0_1-1_mismatch_A-C | | | | | | | | | |
| | 12_1-1_mismatch_A-G | | | | | | | | | |
| | 38_6-6_internal_loop- | | | | | | | | | |
| | symmetric_GGGAGU-UAGGGA | | | | | | | | | |
| 2496 | −33_4-4_bulge-symmetric_UUCG- | GCCACACAGACCUCGCGCAA | −18 | 30 | 0.277 | 0.917 | 0.896 | 1.123 | 1.616 | 1.576 |
| | ACAU | CUUUGAAAGUCCUUUCAUG | | | | | | | | |
| | −18_6-6_internal_loop- | CAUAGAUCCACGGCUACAU | | | | | | | | |
| | symmetric_UGGUGU-UGGAGU | AAUUCCUUUUGGAGUCACU | | | | | | | | |
| | −6_3-3_bulge-symmetric_CAU-CAU | GUCGUACAUUGGCCACUCCC | | | | | | | | |
| | 0_1-1_mismatch_A-C | AGU | | | | | | | | |
| | 6_1-1_mismatch_G-G | | | | | | | | | |
| | 10_1-1_mismatch_U-C | | | | | | | | | |
| | 30_6-6_internal_loop- | | | | | | | | | |
| | symmetric_GCCAAG-GCGCAA | | | | | | | | | |
| | 40_4-4_bulge-symmetric_GAGU- | | | | | | | | | |
| | CAGA | | | | | | | | | |
| 2497 | −33_4-4_bulge-symmetric_UUCG- | GCCACACAGACCUCCUUGAU | −6 | 26 | 0.033 | 0.746 | 0.786 | 0.926 | 1.522 | 1.523 |
| | ACAU | GGAGGAAAGUAGCAUCAUG | | | | | | | | |
| | −20_4-4_bulge-symmetric_UGGU- | UUUACAUCCACGGCUAUGC | | | | | | | | |
| | UAGU | CUGUCCUUUACUAGUCACU | | | | | | | | |
| | −6_6-6_internal_loop- | GUCGUACAUUGGCCACUCCC | | | | | | | | |
| | symmetric_AUUCAU-UGCCUG | AGU | | | | | | | | |
| | 0_1-1_mismatch_A-C | | | | | | | | | |
| | 9_2-2_bulge-symmetric_UU-UU | | | | | | | | | |
| | 16_4-4_bulge-symmetric_AAGG- | | | | | | | | | |
| | AGCA | | | | | | | | | |
| | 26_6-6_internal_loop- | | | | | | | | | |
| | symmetric_AAAGGC-AUGGAG | | | | | | | | | |
| | 40_4-4_bulge-symmetric_GAGU- | | | | | | | | | |
| | CAGA | | | | | | | | | |
| 2498 | −33_4-4_bulge-symmetric_UUCG- | GCCACACAGACCUCCUUGGC | −8 | 24 | 0.031 | 0.493 | 0.537 | 0.936 | 1.295 | 1.321 |
| | ACAU | AUACUUAAGUCCUUUCAUC | | | | | | | | |
| | −20_4-4_bulge-symmetric_UGGU- | AUACAUCCACGGCUAAUUU | | | | | | | | |
| | UAGU | UGGGCUUUACUAGUCACUG | | | | | | | | |
| | −8_6-6_internal_loop- | UCGUACAUUGGCCACUCCCA | | | | | | | | |
| | symmetric_GAAUUC-UUUGGG | GU | | | | | | | | |
| | 0_1-1_mismatch_A-C | | | | | | | | | |
| | 10_2-1_bulge-asymmetric_UC-C | | | | | | | | | |
| | 24_6-6_internal_loop- | | | | | | | | | |
| | symmetric_UCAAAG-AUACUU | | | | | | | | | |
| | 40_4-4_bulge-symmetric_GAGU- | | | | | | | | | |
| | CAGA | | | | | | | | | |
| 2499 | −33_4-4_bulge-symmetric_UUCG- | GCCACACAGACCUCCUAUUA | −8 | 28 | 0.107 | 0.948 | 0.96 | 0.969 | 1.684 | 1.728 |
| | ACAU | GGUUGAAAGUACUUUCAUG | | | | | | | | |
| | −20_4-4_bulge-symmetric_UGGU- | AAUACAUCCACGGCUAAUU | | | | | | | | |
| | UAGU | UAAAACUUUACUAGUCACU | | | | | | | | |
| | −8_6-6_internal_loop- | GUCGUACAUUGGCCACUCCC | | | | | | | | |
| | symmetric_GAAUUC-UUAAAA | AGU | | | | | | | | |
| | 0_1-1_mismatch_A-C | | | | | | | | | |
| | 19_1-1_mismatch_G-A | | | | | | | | | |
| | 28_6-6_internal_loop- | | | | | | | | | |
| | symmetric_AGGCCA-AUUAGG | | | | | | | | | |
| | 40_4-4_bulge-symmetric_GAGU- | | | | | | | | | |
| | CAGA | | | | | | | | | |
| 2500 | −33_4-4_bulge-symmetric_UUCG- | GCCACAUGGGAGUCCUUGG | −10 | 38 | 0.056 | 0.815 | 0.8 | 0.61 | 0.754 | 0.773 |
| | ACAU | CCUUUGAAAGUCCUUUAAU | | | | | | | | |
| | −20_4-4_bulge-symmetric_UGGU- | GAAUACAUCCACGGAAAUG | | | | | | | | |
| | UAGU | AUAUAGCUUACUAGUCACU | | | | | | | | |
| | −10_6-6_internal_loop- | GUCGUACAUUGGCCACUCCC | | | | | | | | |
| | symmetric_AGGAAU-UAUAGC | AGU | | | | | | | | |
| | −3_2-1_bulge-asymmetric_AG-A | | | | | | | | | |
| | 0_1-1_mismatch_A-C | | | | | | | | | |
| | 14_1-1_mismatch_G-A | | | | | | | | | |
| | 38_6-6_internal_loop- | | | | | | | | | |
| | symmetric_GGGAGU-UGGGAG | | | | | | | | | |
| 2501 | −33_4-4_bulge-symmetric_UUCG- | GCCACACAGACCUCAGACAG | −10 | 30 | 0.132 | 0.913 | 0.908 | 0.993 | 1.431 | 1.472 |
| | ACAU | CUUUGAAAGUCCUAUCAUG | | | | | | | | |
| | −20_4-4_bulge-symmetric_UGGU- | AAUAGAUCCACGGCUAAUG | | | | | | | | |
| | UAGU | ACGGUAGUUACUAGUCACU | | | | | | | | |
| | −10_6-6_internal_loop- | GUCGUACAUUGGCCACUCCC | | | | | | | | |
| | symmetric_AGGAAU-CGGUAG | AGU | | | | | | | | |
| | 0_1-1_mismatch_A-C | | | | | | | | | |
| | 6_1-1_mismatch_G-G | | | | | | | | | |
| | 16_1-1_mismatch_A-A | | | | | | | | | |
| | 30_6-6_internal_loop- | | | | | | | | | |
| | symmetric_GCCAAG-AGACAG | | | | | | | | | |
| | 40_4-4_bulge-symmetric_GAGU- | | | | | | | | | |
| | CAGA | | | | | | | | | |
| 2502 | −33_4-4_bulge-symmetric_UUCG- | GCCACAUAGCGGUCCUUGG | −8 | 38 | 0.434 | 0.916 | 0.93 | 0.679 | 0.628 | 0.641 |
| | ACAU | CCUUUGAAAGUCCUUUCAU | | | | | | | | |
| | −20_4-4_bulge-symmetric_UGGU- | GAAUACAACCACGGCUAAU | | | | | | | | |
| | UAGU | UUAGCGCUUUACUAGUCAC | | | | | | | | |
| | −8_6-6_internal_loop- | UGUCGUACAUUGGCCACUC | | | | | | | | |
| | symmetric_GAAUUC-UUAGCG | CCAGU | | | | | | | | |
| | 0_1-1_mismatch_A-C | | | | | | | | | |
| | 4_1-1_mismatch_A-A | | | | | | | | | |
| | 38_6-6_internal_loop- | | | | | | | | | |
| | symmetric_GGGAGU-UAGCGG | | | | | | | | | |
| 2503 | −33_4-4_bulge-symmetric_UUCG- | GCCACAUGGGAGUCCUUGG | −8 | 38 | 0.173 | 0.898 | 0.894 | 0.6 | 0.669 | 0.692 |
| | ACAU | CCUUUGAAAGUCCUUUCAU | | | | | | | | |
| | −20_4-4_bulge-symmetric_UGGU- | GAUACAUCCACGGCUAAUC | | | | | | | | |
| | UAGU | CUUGGCUUUACUAGUCACU | | | | | | | | |
| | −8_6-6_internal_loop- | GUCGUACAUUGGCCACUCCC | | | | | | | | |
| | symmetric_GAAUUC-CCUUGG | AGU | | | | | | | | |
| | 0_1-1_mismatch_A-C | | | | | | | | | |
| | 10_1-0_bulge-asymmetric_U- | | | | | | | | | |
| | 38_6-6_internal_loop- | | | | | | | | | |
| | symmetric_GGGAGU-UGGGAG | | | | | | | | | |
| 2504 | −33_4-4_bulge-symmetric_UUCG- | GCCACACAGACCUCCUACAC | −8 | 28 | 0.018 | 0.828 | 0.846 | 0.941 | 1.632 | 1.613 |
| | ACAU | GGUUGAAAGUCCAUUCAUG | | | | | | | | |
| | −20_4-4_bulge-symmetric_UGGU- | AACAUCCACGGCUAAUUUC | | | | | | | | |
| | UAGU | AAGCUUUACUAGUCACUGU | | | | | | | | |
| | −8_6-6_internal_loop- | CGUACAUUGGCCACUCCCAG | | | | | | | | |
| | symmetric_GAAUUC-UUCAAG | U | | | | | | | | |
| | 0_1-1_mismatch_A-C | | | | | | | | | |
| | 8_2-0_bulge-asymmetric_AU- | | | | | | | | | |
| | 17_1-1_mismatch_A-A | | | | | | | | | |
| | 28_6-6_internal_loop- | | | | | | | | | |
| | symmetric_AGGCCA-ACACGG | | | | | | | | | |
| | 40_4-4_bulge-symmetric_GAGU- | | | | | | | | | |
| | CAGA | | | | | | | | | |
| 2505 | −33_4-4_bulge-symmetric_UUCG- | GCCACACAGACCUCCUGAAU | −6 | 28 | 0.08 | 0.919 | 0.924 | 0.958 | 1.664 | 1.664 |
| | ACAU | ACUUGAAAGUCCUGUCAUG | | | | | | | | |
| | −20_4-4_bulge-symmetric_UGGU- | AAUACAUCCACGACUACCG | | | | | | | | |
| | UAGU | UCAUCCUUUACUAGUCACU | | | | | | | | |
| | −6_6-6_internal_loop- | GUCGUACAUUGGCCACUCCC | | | | | | | | |
| | symmetric_AUUCAU-CCGUCA | AGU | | | | | | | | |
| | −2_1-1_mismatch_C-A | | | | | | | | | |
| | 0_1-1_mismatch_A-C | | | | | | | | | |
| | 16_1-1_mismatch_A-G | | | | | | | | | |
| | 28_6-6_internal_loop- | | | | | | | | | |
| | symmetric_AGGCCA-GAAUAC | | | | | | | | | |
| | 40_4-4_bulge-symmetric_GAGU- | | | | | | | | | |
| | CAGA | | | | | | | | | |
| 2506 | −33_4-4_bulge-symmetric_UUCG- | GCCACACAGACCUCCUUGGC | −18 | 24 | 0.045 | 0.928 | 0.907 | 0.908 | 1.714 | 1.695 |
| | ACAU | GCGAUUAAGUCCCCUUCAU | | | | | | | | |
| | −18_6-6_internal_loop- | GAAUACAUCCACGGGUACU | | | | | | | | |
| | symmetric_UGGUGU-UGUUGU | GAAUUCCUUUUGUUGUCAC | | | | | | | | |
| | −6_1-1_mismatch_U-C | UGUCGUACAUUGGCCACUC | | | | | | | | |
| | −3_1-1_mismatch_G-G | CCAGU | | | | | | | | |
| | 0_1-1_mismatch_A-C | | | | | | | | | |
| | 17_1-2_bulge-asymmetric_A-CC | | | | | | | | | |
| | 24_6-6_internal_loop- | | | | | | | | | |
| | symmetric_UCAAAG-GCGAUU | | | | | | | | | |
| | 40_4-4_bulge-symmetric_GAGU- | | | | | | | | | |
| | CAGA | | | | | | | | | |
| 2507 | −33_4-4_bulge-symmetric_UUCG- | GCCACACAGACCUCCUUGGC | −8 | 24 | 0.03 | 0.619 | 0.684 | 0.932 | 1.427 | 1.439 |
| | ACAU | AGCGAUAAGUCCGUUCAUG | | | | | | | | |
| | −20_4-4_bulge-symmetric_UGGU- | AAAAGAUCCACGGCUAAUC | | | | | | | | |
| | UAGU | CACGACUUUACUAGUCACU | | | | | | | | |
| | −8_6-6_internal_loop- | GUCGUACAUUGGCCACUCCC | | | | | | | | |
| | symmetric_GAAUUC-CCACGA | AGU | | | | | | | | |
| | 0_1-1_mismatch_A-C | | | | | | | | | |
| | 6_1-1_mismatch_G-G | | | | | | | | | |
| | 8_1-1_mismatch_A-A | | | | | | | | | |
| | 17_1-1_mismatch_A-G | | | | | | | | | |
| | 24_6-6_internal_loop- | | | | | | | | | |
| | symmetric_UCAAAG-AGCGAU | | | | | | | | | |
| | 40_4-4_bulge-symmetric_GAGU- | | | | | | | | | |
| | CAGA | | | | | | | | | |
| 2508 | −33_4-4_bulge-symmetric_UUCG- | GCCACACAGACCUCGAACGA | −8 | 30 | 0.401 | 0.881 | 0.88 | 1.27 | 1.516 | 1.585 |
| | ACAU | CUUUGAAAGUCCUUUCAUG | | | | | | | | |
| | −20_4-4_bulge-symmetric_UGGU- | AAUAGAUCCACGGCUAAUU | | | | | | | | |
| | UAGU | AUAAGCUUUACUAGUCACU | | | | | | | | |
| | −8_6-6_internal_loop- | GUCGUACAUUGGCCACUCCC | | | | | | | | |
| | symmetric_GAAUUC-UAUAAG | AGU | | | | | | | | |
| | 0_1-1_mismatch_A-C | | | | | | | | | |
| | 6_1-1_mismatch_G-G | | | | | | | | | |
| | 30_6-6_internal_loop- | | | | | | | | | |
| | symmetric_GCCAAG-GAACGA | | | | | | | | | |
| | 40_4-4_bulge-symmetric_GAGU- | | | | | | | | | |
| | CAGA | | | | | | | | | |
| 2509 | −8_6-6_internal_loop- | GCCACACAGACCUCGAACGA | −8 | 30 | 0.661 | 0.902 | 0.841 | 1.38 | 1.465 | 1.491 |
| | symmetric_GAAUUC-UAUAAG | CUUUGAAAGUCCUUUCAUG | | | | | | | | |
| | 0_1-1_mismatch_A-C | AAUAGAUCCACGGCUAAUU | | | | | | | | |
| | 6_1-1_mismatch_G-G | AUAAGCUUUACACCACACU | | | | | | | | |
| | 30_6-6_internal_loop- | GUCGUCGAAUGGCCACUCCC | | | | | | | | |
| | symmetric_GCCAAG-GAACGA | AGU | | | | | | | | |
| | 40_4-4_bulge-symmetric_GAGU- | | | | | | | | | |
| | CAGA | | | | | | | | | |
| 2510 | −33_4-4_bulge-symmetric_UUCG- | GCCACACAGACCUCCUUGGC | −10 | 24 | 0.12 | 0.805 | 0.835 | 1.019 | 1.621 | 1.57 |
| | ACAU | GAUCACAAGUCCUUUCAUG | | | | | | | | |
| | −20_4-4_bulge-symmetric_UGGU- | AAUAAAUCCACGGUUAAUG | | | | | | | | |
| | UAGU | ACCAGUGUUACUAGUCACU | | | | | | | | |
| | −10_6-6_internal_loop- | GUCGUACAUUGGCCACUCCC | | | | | | | | |
| | symmetric_AGGAAU-CCAGUG | AGU | | | | | | | | |
| | −3_1-1_wobble_G-U | | | | | | | | | |
| | 0_1-1_mismatch_A-C | | | | | | | | | |
| | 6_1-1_mismatch_G-A | | | | | | | | | |
| | 24_6-6_internal_loop- | | | | | | | | | |
| | symmetric_UCAAAG-GAUCAC | | | | | | | | | |
| | 40_4-4_bulge-symmetric_GAGU- | | | | | | | | | |
| | CAGA | | | | | | | | | |
| 2511 | −33_4-4_bulge-symmetric_UUCG- | GCCACACAGACCAGGUAUG | −18 | 32 | 0.042 | 0.596 | 0.654 | 0.956 | 1.407 | 1.418 |
| | ACAU | CCUUUGAAAGUAGUUCAUG | | | | | | | | |
| | −18_6-6_internal_loop- | AAUACAACCACGGCUACAC | | | | | | | | |
| | symmetric_UGGUGU-CUUGGU | AAUUCCUUUCUUGGUCACU | | | | | | | | |
| | −6_3-3_bulge-symmetric_CAU-CAC | GUCGUACAUUGGCCACUCCC | | | | | | | | |
| | 0_1-1_mismatch_A-C | AGU | | | | | | | | |
| | 4_1-1_mismatch_A-A | | | | | | | | | |
| | 17_3-2_bulge-asymmetric_AGG-AG | | | | | | | | | |
| | 32_6-6_internal_loop- | | | | | | | | | |
| | symmetric_CAAGGA-AGGUAU | | | | | | | | | |
| | 40_4-4_bulge-symmetric_GAGU- | | | | | | | | | |
| | CAGA | | | | | | | | | |
| 2512 | −33_4-4_bulge-symmetric_UUCG- | GCCACACAGACCUCCUUGUU | −8 | 26 | 0.048 | 0.667 | 0.674 | 0.975 | 1.506 | 1.521 |
| | ACAU | GAAGGAAAGUCCUUCGUGA | | | | | | | | |
| | −20_4-4_bulge-symmetric_UGGU- | AUACAUCCACGGCUAAUCU | | | | | | | | |
| | UAGU | GGGGCUUUACUAGUCACUG | | | | | | | | |
| | −8_6-6_internal_loop- | UCGUACAUUGGCCACUCCCA | | | | | | | | |
| | symmetric_GAAUUC-CUGGGG | GU | | | | | | | | |
| | 0_1-1_mismatch_A-C | | | | | | | | | |
| | 13_1-1_wobble_U-G | | | | | | | | | |
| | 17_1-0_bulge-asymmetric_A- | | | | | | | | | |
| | 26_6-6_internal_loop- | | | | | | | | | |
| | symmetric_AAAGGC-UUGAAG | | | | | | | | | |
| | 40_4-4_bulge-symmetric_GAGU- | | | | | | | | | |
| | CAGA | | | | | | | | | |
| 2513 | −33_4-4_bulge-symmetric_UUCG- | GCCACACAGACCUCCUUGUA | −10 | 26 | 0.171 | 0.833 | 0.816 | 0.886 | 1.332 | 1.275 |
| | ACAU | GGAGGAAAGUCCUUUCAUG | | | | | | | | |
| | −20_4-4_bulge-symmetric_UGGU- | AAUACAUGCACGGCUAAUG | | | | | | | | |
| | UAGU | AUCGGUGUUACUAGUCACU | | | | | | | | |
| | −10_6-6_internal_loop- | GUCGUACAUUGGCCACUCCC | | | | | | | | |
| | symmetric_AGGAAU-UCGGUG | AGU | | | | | | | | |
| | 0_1-1_mismatch_A-C | | | | | | | | | |
| | 3_1-1_mismatch_G-G | | | | | | | | | |
| | 26_6-6_internal_loop- | | | | | | | | | |
| | symmetric_AAAGGC-UAGGAG | | | | | | | | | |
| | 40_4-4_bulge-symmetric_GAGU- | | | | | | | | | |
| | CAGA | | | | | | | | | |
| 2514 | −33_4-4_bulge-symmetric_UUCG- | GCCACACAGACCAUAGACGC | −18 | 32 | 0.193 | 0.934 | 0.939 | 0.896 | 1.261 | 1.351 |
| | ACAU | CUUUGAAAGUGCUUUCAUG | | | | | | | | |
| | −18_6-6_internal_loop- | AAUACAUCCACGGCUACUG | | | | | | | | |
| | symmetric_UGGUGU-CUUGGU | AAUUCCUUUCUUGGUCACU | | | | | | | | |
| | −6_1-1_mismatch_U-C | GUCGUACAUUGGCCACUCCC | | | | | | | | |
| | 0_1-1_mismatch_A-C | AGU | | | | | | | | |
| | 19_1-1_mismatch_G-G | | | | | | | | | |
| | 32_6-6_internal_loop- | | | | | | | | | |
| | symmetric_CAAGGA-AUAGAC | | | | | | | | | |
| | 40_4-4_bulge-symmetric_GAGU- | | | | | | | | | |
| | CAGA | | | | | | | | | |
| 2515 | −33_4-4_bulge-symmetric_UUCG- | GCCACACAGACCUCCUUGGC | −6 | 24 | 0.065 | 0.919 | 0.91 | 0.957 | 1.698 | 1.683 |
| | ACAU | GGCAUCAAGUAAUUUCAUG | | | | | | | | |
| | −20_4-4_bulge-symmetric_UGGU- | AAUACAUCCACGGCUACGA | | | | | | | | |
| | UAGU | CGGUCCUUUACUAGUCACU | | | | | | | | |
| | −6_6-6_internal_loop- | GUCGUACAUUGGCCACUCCC | | | | | | | | |
| | symmetric_AUUCAU-CGACGG | AGU | | | | | | | | |
| | 0_1-1_mismatch_A-C | | | | | | | | | |
| | 18_2-2_bulge-symmetric_GG-AA | | | | | | | | | |
| | 24_6-6_internal_loop- | | | | | | | | | |
| | symmetric_UCAAAG-GGCAUC | | | | | | | | | |
| | 40_4-4_bulge-symmetric_GAGU- | | | | | | | | | |
| | CAGA | | | | | | | | | |
| 2516 | −33_4-4_bulge-symmetric_UUCG- | GCCACACAGACCGGAAAUG | −10 | 32 | 0.036 | 0.529 | 0.578 | 0.914 | 1.32 | 1.311 |
| | ACAU | CCUUUGAAAGUCCCGCCAU | | | | | | | | |
| | −20_4-4_bulge-symmetric_UGGU- | GAAUAAUUCCACGGCUAUG | | | | | | | | |
| | UAGU | GACGAUGGUUACUAGUCAC | | | | | | | | |
| | −10_6-6_internal_loop- | UGUCGUACAUUGGCCACUC | | | | | | | | |
| | symmetric_AGGAAU-CGAUGG | CCAGU | | | | | | | | |
| | −6_2-2_bulge-symmetric_AU-UG | | | | | | | | | |
| | 0_1-1_mismatch_A-C | | | | | | | | | |
| | 5_2-2_bulge-symmetric_UG-AU | | | | | | | | | |
| | 15_3-3_bulge-symmetric_AAA-CGC | | | | | | | | | |
| | 32_6-6_internal_loop- | | | | | | | | | |
| | symmetric_CAAGGA-GGAAAU | | | | | | | | | |
| | 40_4-4_bulge-symmetric_GAGU- | | | | | | | | | |
| | CAGA | | | | | | | | | |
| 2517 | −33_4-4_bulge-symmetric_UUCG- | GCCACACAGACCUCCUUGGC | −8 | 24 | 0.033 | 0.888 | 0.883 | 0.921 | 1.638 | 1.596 |
| | ACAU | GGCGCCAAGUGAUUUGAUG | | | | | | | | |
| | −20_4-4_bulge-symmetric_UGGU- | AAUACAUCCACGGCUAGUU | | | | | | | | |
| | UAGU | UCCAACUUUACUAGUCACU | | | | | | | | |
| | −8_6-6_internal_loop- | GUCGUACAUUGGCCACUCCC | | | | | | | | |
| | symmetric_GAAUUC-UUCCAA | AGU | | | | | | | | |
| | −6_1-1_wobble_U-G | | | | | | | | | |
| | 0_1-1_mismatch_A-C | | | | | | | | | |
| | 14_1-1_mismatch_G-G | | | | | | | | | |
| | 18_2-2_bulge-symmetric_GG-GA | | | | | | | | | |
| | 24_6-6_internal_loop- | | | | | | | | | |
| | symmetric_UCAAAG-GGCGCC | | | | | | | | | |
| | 40_4-4_bulge-symmetric_GAGU- | | | | | | | | | |
| | CAGA | | | | | | | | | |
| 2518 | −33_4-4_bulge-symmetric_UUCG- | GCCACACAGACCUCCUUGGC | −12 | 24 | 0.025 | 0.835 | 0.813 | 0.916 | 1.552 | 1.472 |
| | ACAU | ACGGUUAAGUCCUUCGAUG | | | | | | | | |
| | −20_4-4_bulge-symmetric_UGGU- | AAUACGUCCACGGCUUUUG | | | | | | | | |
| | UAGU | AAUGCAUCGACUAGUCACU | | | | | | | | |
| | −12_6-6_internal_loop- | GUCGUACAUUGGCCACUCCC | | | | | | | | |
| | symmetric_AAAGGA-GCAUCG | AGU | | | | | | | | |
| | −5_2-2_bulge-symmetric_UU-UU | | | | | | | | | |
| | 0_1-1_mismatch_A-C | | | | | | | | | |
| | 5_1-1_wobble_U-G | | | | | | | | | |
| | 14_2-2_bulge-symmetric_GA-CG | | | | | | | | | |
| | 24_6-6_internal_loop- | | | | | | | | | |
| | symmetric_UCAAAG-ACGGUU | | | | | | | | | |
| | 40_4-4_bulge-symmetric_GAGU- | | | | | | | | | |
| | CAGA | | | | | | | | | |
| 2519 | −33_4-4_bulge-symmetric_UUCG- | GCCACACAGACCUCCUUGAU | −8 | 26 | 0.177 | 0.901 | 0.889 | 0.964 | 1.635 | 1.579 |
| | ACAU | AGUGGAAAGUCCUUUCAUG | | | | | | | | |
| | −20_4-4_bulge-symmetric_UGGU- | AAUACAUCCACGGGUAAUC | | | | | | | | |
| | UAGU | GCCGACUUUACUAGUCACU | | | | | | | | |
| | −8_6-6_internal_loop- | GUCGUACAUUGGCCACUCCC | | | | | | | | |
| | symmetric_GAAUUC-CGCCGA | AGU | | | | | | | | |
| | −3_1-1_mismatch_G-G | | | | | | | | | |
| | 0_1-1_mismatch_A-C | | | | | | | | | |
| | 26_6-6_internal_loop- | | | | | | | | | |
| | symmetric_AAAGGC-AUAGUG | | | | | | | | | |
| | 40_4-4_bulge-symmetric_GAGU- | | | | | | | | | |
| | CAGA | | | | | | | | | |
| 2520 | −33_4-4_bulge-symmetric_UUCG- | GCCACACAGACCUCAAGAU | −20 | 30 | 0.248 | 0.945 | 0.928 | 1.104 | 1.55 | 1.561 |
| | ACAU | ACUUUGAAAGUCCUUUCAU | | | | | | | | |
| | −20_6-6_internal_loop- | GCAUACAUUCACGGCUAAU | | | | | | | | |
| | symmetric_UGUGGU-UGAUUU | GAAUUCCUUUACUGAUUUC | | | | | | | | |
| | 0_1-1_mismatch_A-C | UGUCGUACAUUGGCCACUC | | | | | | | | |
| | 3_1-1_wobble_G-U | CCAGU | | | | | | | | |
| | 10_1-1_mismatch_U-C | | | | | | | | | |
| | 30_6-6_internal_loop- | | | | | | | | | |
| | symmetric_GCCAAG-AAGAUA | | | | | | | | | |
| | 40_4-4_bulge-symmetric_GAGU- | | | | | | | | | |
| | CAGA | | | | | | | | | |
| 2521 | −33_4-4_bulge-symmetric_UUCG- | GCCACAUGGGCAUCCUUGG | −4 | 38 | 0.4 | 0.926 | 0.912 | 0.86 | 0.74 | 0.794 |
| | ACAU | CCUUUGAAAGUCCUUUCAU | | | | | | | | |
| | −20_4-4_bulge-symmetric_UGGU- | GUAUACAUCCACGGCAGUU | | | | | | | | |
| | UAGU | ACAUUCCUUUACUAGUCAC | | | | | | | | |
| | −4_6-6_internal_loop- | UGUCGUACAUUGGCCACUC | | | | | | | | |
| | symmetric_UCAUUA-AGUUAC | CCAGU | | | | | | | | |
| | 0_1-1_mismatch_A-C | | | | | | | | | |
| | 10_1-1_mismatch_U-U | | | | | | | | | |
| | 38_6-6_internal_loop- | | | | | | | | | |
| | symmetric_GGGAGU-UGGGCA | | | | | | | | | |
| 2522 | −33_4-4_bulge-symmetric_UUCG- | GCCACACAGACCUCCUUGGC | −6 | 24 | 0.047 | 0.808 | 0.799 | 0.96 | 1.627 | 1.578 |
| | ACAU | GACAACAAGUCCUCUCAUG | | | | | | | | |
| | −20_4-4_bulge-symmetric_UGGU- | AAUAAUUCCACGGCUACCG | | | | | | | | |
| | UAGU | UUCUCCUUUACUAGUCACU | | | | | | | | |
| | −6_6-6_internal_loop- | GUCGUACAUUGGCCACUCCC | | | | | | | | |
| | symmetric_AUUCAU-CCGUUC | AGU | | | | | | | | |
| | 0_1-1_mismatch_A-C | | | | | | | | | |
| | 5_2-2_bulge-symmetric_UG-AU | | | | | | | | | |
| | 16_1-1_mismatch_A-C | | | | | | | | | |
| | 24_6-6_internal_loop- | | | | | | | | | |
| | symmetric_UCAAAG-GACAAC | | | | | | | | | |
| | 40_4-4_bulge-symmetric_GAGU- | | | | | | | | | |
| | CAGA | | | | | | | | | |
| 2523 | −33_4-4_bulge-symmetric_UUCG- | GCCACAUGGGGGUCCUUGG | −10 | 38 | 0.254 | 0.843 | 0.835 | 0.487 | 0.701 | 0.671 |
| | ACAU | CCUUUGAAAGUCCUUUCAU | | | | | | | | |
| | −20_4-4_bulge-symmetric_UGGU- | GAAUAUAUCCACGGCUAAU | | | | | | | | |
| | UAGU | GACGAUUAUUACUAGUCAC | | | | | | | | |
| | −10_6-6_internal_loop- | UGUCGUACAUUGGCCACUC | | | | | | | | |
| | symmetric_AGGAAU-CGAUUA | CCAGU | | | | | | | | |
| | 0_1-1_mismatch_A-C | | | | | | | | | |
| | 6_1-1_wobble_G-U | | | | | | | | | |
| | 38_6-6_internal_loop- | | | | | | | | | |
| | symmetric_GGGAGU-UGGGGG | | | | | | | | | |
| 2524 | −33_4-4_bulge-symmetric_UUCG- | GCCACACAGACCUCCUUGUA | −6 | 26 | 0.202 | 0.838 | 0.871 | 1.07 | 1.589 | 1.634 |
| | ACAU | GUGCGAAAGUCCUUUCAUG | | | | | | | | |
| | −20_4-4_bulge-symmetric_UGGU- | AAUAAGAUCCACGGCUACG | | | | | | | | |
| | UAGU | UAACUCCUUUACUAGUCAC | | | | | | | | |
| | −6_6-6_internal_loop- | UGUCGUACAUUGGCCACUC | | | | | | | | |
| | symmetric_AUUCAU-CGUAAC | CCAGU | | | | | | | | |
| | 0_1-1_mismatch_A-C | | | | | | | | | |
| | 6_1-2_bulge-asymmetric_G-AG | | | | | | | | | |
| | 26_6-6_internal_loop- | | | | | | | | | |
| | symmetric_AAAGGC-UAGUGC | | | | | | | | | |
| | 40_4-4_bulge-symmetric_GAGU- | | | | | | | | | |
| | CAGA | | | | | | | | | |
| 2525 | −33_4-4_bulge-symmetric_UUCG- | GCCACACAGACCUCCUAAUA | −18 | 28 | 0.076 | 0.905 | 0.913 | 0.913 | 1.561 | 1.65 |
| | ACAU | AAUUGAAAGUCCUUUCAAG | | | | | | | | |
| | −18_6-6_internal_loop- | AAUACAUCCACGGCUAAUG | | | | | | | | |
| | symmetric_UGGUGU-UUGUUU | AAUUCCUUUUUGUUUCACU | | | | | | | | |
| | 0_1-1_mismatch_A-C | GUCGUACAUUGGCCACUCCC | | | | | | | | |
| | 12_1-1_mismatch_A-A | AGU | | | | | | | | |
| | 28_6-6_internal_loop- | | | | | | | | | |
| | symmetric_AGGCCA-AAUAAA | | | | | | | | | |
| | 40_4-4_bulge-symmetric_GAGU- | | | | | | | | | |
| | CAGA | | | | | | | | | |
| 2526 | −33_4-4_bulge-symmetric_UUCG- | GCCACACAGACCUCCUUGAA | −12 | 26 | 0.021 | 0.624 | 0.64 | 0.931 | 1.4 | 1.365 |
| | ACAU | UAGGGAAAGUCCAUAUUCA | | | | | | | | |
| | −20_4-4_bulge-symmetric_UGGU- | UGAAUACCCCCACGGCUAA | | | | | | | | |
| | UAGU | UGAAUGGACCGACUAGUCA | | | | | | | | |
| | −12_6-6_internal_loop- | CUGUCGUACAUUGGCCACU | | | | | | | | |
| | symmetric_AAAGGA-GGACCG | CCCAGU | | | | | | | | |
| | 0_1-1_mismatch_A-C | | | | | | | | | |
| | 4_2-2_bulge-symmetric_AU-CC | | | | | | | | | |
| | 17_1-3_bulge-asymmetric_A-AUA | | | | | | | | | |
| | 26_6-6_internal_loop- | | | | | | | | | |
| | symmetric_AAAGGC-AAUAGG | | | | | | | | | |
| | 40_4-4_bulge-symmetric_GAGU- | | | | | | | | | |
| | CAGA | | | | | | | | | |
| 2527 | −33_4-4_bulge-symmetric_UUCG- | GCCACACAGACCAAUGCUGC | −8 | 32 | 0.06 | 0.728 | 0.792 | 0.921 | 1.176 | 1.22 |
| | ACAU | CUUUGAAAGUCCUUUGCAU | | | | | | | | |
| | −20_4-4_bulge-symmetric_UGGU- | CAUUACAUCCACGGCUAAU | | | | | | | | |
| | UAGU | AGGCGGCUUUACUAGUCAC | | | | | | | | |
| | −8_6-6_internal_loop- | UGUCGUACAUUGGCCACUC | | | | | | | | |
| | symmetric_GAAUUC-AGGCGG | CCAGU | | | | | | | | |
| | 0_1-1_mismatch_A-C | | | | | | | | | |
| | 9_3-3_bulge-symmetric_UUC-CAU | | | | | | | | | |
| | 14_0-1_bulge-asymmetric_-G | | | | | | | | | |
| | 32_6-6_internal_loop- | | | | | | | | | |
| | symmetric_CAAGGA-AAUGCU | | | | | | | | | |
| | 40_4-4_bulge-symmetric_GAGU- | | | | | | | | | |
| | CAGA | | | | | | | | | |
| 2528 | −33_4-4_bulge-symmetric_UUCG- | GCCACACAGACCUCCUCUCA | −18 | 28 | 0.022 | 0.928 | 0.919 | 0.923 | 1.705 | 1.664 |
| | ACAU | UCUUGAAAGUCGACUUUCA | | | | | | | | |
| | −18_6-6_internal_loop- | UGAAUACAUCCACGGAAUG | | | | | | | | |
| | symmetric_UGGUGU-UGUAAU | AAUUCCUUUUGUAAUCACU | | | | | | | | |
| | −3_2-0_bulge-asymmetric_AG- | GUCGUACAUUGGCCACUCCC | | | | | | | | |
| | 0_1-1_mismatch_A-C | AGU | | | | | | | | |
| | 18_0-2_bulge-asymmetric_-GA | | | | | | | | | |
| | 28_6-6_internal_loop- | | | | | | | | | |
| | symmetric_AGGCCA-CUCAUC | | | | | | | | | |
| | 40_4-4_bulge-symmetric_GAGU- | | | | | | | | | |
| | CAGA | | | | | | | | | |
| 2529 | −33_4-4_bulge-symmetric_UUCG- | GCCACACAGACCUCCUCUCG | −6 | 28 | 0.016 | 0.716 | 0.791 | 0.939 | 1.544 | 1.603 |
| | ACAU | ACUUGAAAGUCCCUUUCAU | | | | | | | | |
| | −20_4-4_bulge-symmetric_UGGU- | GAAUUUCUCCACGGCUACG | | | | | | | | |
| | UAGU | ACUGUCCUUUACUAGUCAC | | | | | | | | |
| | −6_6-6_internal_loop- | UGUCGUACAUUGGCCACUC | | | | | | | | |
| | symmetric_AUUCAU-CGACUG | CCAGU | | | | | | | | |
| | 0_1-1_mismatch_A-C | | | | | | | | | |
| | 5_3-3_bulge-symmetric_UGU-UUC | | | | | | | | | |
| | 19_0-1_bulge-asymmetric_-C | | | | | | | | | |
| | 28_6-6_internal_loop- | | | | | | | | | |
| | symmetric_AGGCCA-CUCGAC | | | | | | | | | |
| | 40_4-4_bulge-symmetric_GAGU- | | | | | | | | | |
| | CAGA | | | | | | | | | |
| 2530 | −33_4-4_bulge-symmetric_UUCG- | GCCACACAGACCUCCUUGUA | −8 | 26 | 0.033 | 0.775 | 0.765 | 0.913 | 1.564 | 1.514 |
| | ACAU | UAUCGAAAGUCCUUUAAUG | | | | | | | | |
| | −20_4-4_bulge-symmetric_UGGU- | AAUAGAAUCCACGGCUAAU | | | | | | | | |
| | UAGU | CCUUGGCUUUACUAGUCAC | | | | | | | | |
| | −8_6-6_internal_loop- | UGUCGUACAUUGGCCACUC | | | | | | | | |
| | symmetric_GAAUUC-CCUUGG | CCAGU | | | | | | | | |
| | 0_1-1_mismatch_A-C | | | | | | | | | |
| | 6_1-2_bulge-asymmetric_G-GA | | | | | | | | | |
| | 14_1-1_mismatch_G-A | | | | | | | | | |
| | 26_6-6_internal_loop- | | | | | | | | | |
| | symmetric_AAAGGC-UAUAUC | | | | | | | | | |
| | 40_4-4_bulge-symmetric_GAGU- | | | | | | | | | |
| | CAGA | | | | | | | | | |
| 2531 | −33_4-4_bulge-symmetric_UUCG- | GCCACACAGACCUCGCAUCA | −6 | 30 | 0.091 | 0.898 | 0.888 | 0.985 | 1.545 | 1.603 |
| | ACAU | CUUUGAAAGUACUUUCAUU | | | | | | | | |
| | −20_4-4_bulge-symmetric_UGGU- | AAUACAUCCACGGCUACGU | | | | | | | | |
| | UAGU | CGCUCCUUUACUAGUCACU | | | | | | | | |
| | −6_6-6_internal_loop- | GUCGUACAUUGGCCACUCCC | | | | | | | | |
| | symmetric_AUUCAU-CGUCGC | AGU | | | | | | | | |
| | 0_1-1_mismatch_A-C | | | | | | | | | |
| | 11_1-1_mismatch_C-U | | | | | | | | | |
| | 19_1-1_mismatch_G-A | | | | | | | | | |
| | 30_6-6_internal_loop- | | | | | | | | | |
| | symmetric_GCCAAG-GCAUCA | | | | | | | | | |
| | 40_4-4_bulge-symmetric_GAGU- | | | | | | | | | |
| | CAGA | | | | | | | | | |
| 2532 | −33_4-4_bulge-symmetric_UUCG- | GCCACACAGACCUCCUUGGC | −10 | 24 | 0.197 | 0.867 | 0.855 | 1.056 | 1.612 | 1.515 |
| | ACAU | AAGUCCAAGUCCUUUCAUC | | | | | | | | |
| | −20_4-4_bulge-symmetric_UGGU- | AAUACAUCCACGGCCAUGA | | | | | | | | |
| | UAGU | CGCGGGUUACUAGUCACUG | | | | | | | | |
| | −10_6-6_internal_loop- | UCGUACAUUGGCCACUCCCA | | | | | | | | |
| | symmetric_AGGAAU-CGCGGG | GU | | | | | | | | |
| | −4_2-1_bulge-asymmetric_UA-C | | | | | | | | | |
| | 0_1-1_mismatch_A-C | | | | | | | | | |
| | 11_1-1_mismatch_C-C | | | | | | | | | |
| | 24_6-6_internal_loop- | | | | | | | | | |
| | symmetric_UCAAAG-AAGUCC | | | | | | | | | |
| | 40_4-4_bulge-symmetric_GAGU- | | | | | | | | | |
| | CAGA | | | | | | | | | |
| 2533 | −33_4-4_bulge-symmetric_UUCG- | GCCACACAGACCAGAAGUG | −8 | 32 | 0.452 | 0.943 | 0.923 | 1.176 | 1.121 | 1.198 |
| | ACAU | CCUUUGAAAGUCCUUUCAU | | | | | | | | |
| | −20_4-4_bulge-symmetric_UGGU- | GAAUACAACCACGGCUAAU | | | | | | | | |
| | UAGU | UUGGUACUUUACUAGUCAC | | | | | | | | |
| | −8_6-6_internal_loop- | UGUCGUACAUUGGCCACUC | | | | | | | | |
| | symmetric_GAAUUC-UUGGUA | CCAGU | | | | | | | | |
| | 0_1-1_mismatch_A-C | | | | | | | | | |
| | 4_1-1_mismatch_A-A | | | | | | | | | |
| | 32_6-6_internal_loop- | | | | | | | | | |
| | symmetric_CAAGGA-AGAAGU | | | | | | | | | |
| | 40_4-4_bulge-symmetric_GAGU- | | | | | | | | | |
| | CAGA | | | | | | | | | |
| 2534 | −33_4-4_bulge-symmetric_UUCG- | GCCACACAGACCUCCUAAUA | −10 | 28 | 0.137 | 0.808 | 0.822 | 1.026 | 1.582 | 1.603 |
| | ACAU | GGUUGAAAGUCCUUUCGUA | | | | | | | | |
| | −20_4-4_bulge-symmetric_UGGU- | AAUACAUCCACGGCUAAUA | | | | | | | | |
| | UAGU | GAUGAGGCUUACUAGUCAC | | | | | | | | |
| | −10_6-6_internal_loop- | UGUCGUACAUUGGCCACUC | | | | | | | | |
| | symmetric_AGGAAU-UGAGGC | CCAGU | | | | | | | | |
| | −7_0-1_bulge-asymmetric_-A | | | | | | | | | |
| | 0_1-1_mismatch_A-C | | | | | | | | | |
| | 11_1-1_mismatch_C-A | | | | | | | | | |
| | 13_1-1_wobble_U-G | | | | | | | | | |
| | 28_6-6_internal_loop- | | | | | | | | | |
| | symmetric_AGGCCA-AAUAGG | | | | | | | | | |
| | 40_4-4_bulge-symmetric_GAGU- | | | | | | | | | |
| | CAGA | | | | | | | | | |
| 2535 | −33_4-4_bulge-symmetric_UUCG- | GCCACACAGACCUCCUUGGC | −8 | 24 | 0.233 | 0.832 | 0.827 | 1.09 | 1.541 | 1.57 |
| | ACAU | GCGCAUAAGUCCUUUCAUG | | | | | | | | |
| | −20_4-4_bulge-symmetric_UGGU- | AAUACCUCCACGGCUAAUC | | | | | | | | |
| | UAGU | GCCCGCUUUACUAGUCACU | | | | | | | | |
| | −8_6-6_internal_loop- | GUCGUACAUUGGCCACUCCC | | | | | | | | |
| | symmetric_GAAUUC-CGCCCG | AGU | | | | | | | | |
| | 0_1-1_mismatch_A-C | | | | | | | | | |
| | 5_1-1_mismatch_U-C | | | | | | | | | |
| | 24_6-6_internal_loop- | | | | | | | | | |
| | symmetric_UCAAAG-GCGCAU | | | | | | | | | |
| | 40_4-4_bulge-symmetric_GAGU- | | | | | | | | | |
| | CAGA | | | | | | | | | |
| 2536 | −33_4-4_bulge-symmetric_UUCG- | GCCACACAGACCUCCUUGAU | −20 | 26 | 0.118 | 0.919 | 0.94 | 1.031 | 1.738 | 1.768 |
| | ACAU | GGCGGAAAGUCCCUUCAUG | | | | | | | | |
| | −20_6-6_internal_loop- | AAUACUAUCCACGGCUCUA | | | | | | | | |
| | symmetric_UGUGGU-UGGAGU | CAAUUCCUUUACUGGAGUC | | | | | | | | |
| | −5_4-4_bulge-symmetric_CAUU- | UGUCGUACAUUGGCCACUC | | | | | | | | |
| | CUAC | CCAGU | | | | | | | | |
| | 0_1-1_mismatch_A-C | | | | | | | | | |
| | 5_0-1_bulge-asymmetric_-U | | | | | | | | | |
| | 17_1-1_mismatch_A-C | | | | | | | | | |
| | 26_6-6_internal_loop- | | | | | | | | | |
| | symmetric_AAAGGC-AUGGCG | | | | | | | | | |
| | 40_4-4_bulge-symmetric_GAGU- | | | | | | | | | |
| | CAGA | | | | | | | | | |
| 2537 | −33_4-4_bulge-symmetric_UUCG- | GCCACACAGACCUCCUAUAG | −8 | 28 | 0.377 | 0.895 | 0.887 | 1.199 | 1.571 | 1.605 |
| | ACAU | AGUUGAAAGUCCUUUCAUG | | | | | | | | |
| | −20_4-4_bulge-symmetric_UGGU- | AAUACAUCUACGGCUAAUC | | | | | | | | |
| | UAGU | GCCGACUUUACUAGUCACU | | | | | | | | |
| | −8_6-6_internal_loop- | GUCGUACAUUGGCCACUCCC | | | | | | | | |
| | symmetric_GAAUUC-CGCCGA | AGU | | | | | | | | |
| | 0_1-1_mismatch_A-C | | | | | | | | | |
| | 2_1-1_wobble_G-U | | | | | | | | | |
| | 28_6-6_internal_loop- | | | | | | | | | |
| | symmetric_AGGCCA-AUAGAG | | | | | | | | | |
| | 40_4-4_bulge-symmetric_GAGU- | | | | | | | | | |
| | CAGA | | | | | | | | | |
| 2538 | −33_4-4_bulge-symmetric_UUCG- | GCCACACAGACCUCCUCAUG | −18 | 28 | 0.242 | 0.93 | 0.915 | 1.071 | 1.577 | 1.634 |
| | ACAU | AAUUGAAAGUCCUUUCAUG | | | | | | | | |
| | −18_6-6_internal_loop- | AAUACAGACACGGCUAAUG | | | | | | | | |
| | symmetric_UGGUGU-UGUUUU | AAUUCCUUUUGUUUUCACU | | | | | | | | |
| | 0_1-1_mismatch_A-C | GUCGUACAUUGGCCACUCCC | | | | | | | | |
| | 3_2-2_bulge-symmetric_GA-GA | AGU | | | | | | | | |
| | 28_6-6_internal_loop- | | | | | | | | | |
| | symmetric_AGGCCA-CAUGAA | | | | | | | | | |
| | 40_4-4_bulge-symmetric_GAGU- | | | | | | | | | |
| | CAGA | | | | | | | | | |
| 2539 | −33_4-4_bulge-symmetric_UUCG- | GCCACACAGACCUCCUACAA | −18 | 28 | 0.014 | 0.803 | 0.827 | 0.953 | 1.604 | 1.612 |
| | ACAU | GGUUGAAAGUCCUUACAUG | | | | | | | | |
| | −18_6-6_internal_loop- | AAUUCAUCCACGUGUAUUG | | | | | | | | |
| | symmetric_UGGUGU-UGAGGU | AAUUCCUUUUGAGGUCACU | | | | | | | | |
| | −6_1-1_mismatch_U-U | GUCGUACAUUGGCCACUCCC | | | | | | | | |
| | −2_2-2_bulge-symmetric_GC-UG | AGU | | | | | | | | |
| | 0_1-1_mismatch_A-C | | | | | | | | | |
| | 7_1-1_mismatch_U-U | | | | | | | | | |
| | 15_1-1_mismatch_A-A | | | | | | | | | |
| | 28_6-6_internal_loop- | | | | | | | | | |
| | symmetric_AGGCCA-ACAAGG | | | | | | | | | |
| | 40_4-4_bulge-symmetric_GAGU- | | | | | | | | | |
| | CAGA | | | | | | | | | |
| 2540 | −33_4-4_bulge-symmetric_UUCG- | GCCACACAGACCUCCUUGGC | −8 | 24 | 0.093 | 0.854 | 0.873 | 0.93 | 1.673 | 1.631 |
| | ACAU | GACGCCAAGUCCUUUCAUG | | | | | | | | |
| | −20_4-4_bulge-symmetric_UGGU- | AAUACAUCCACGGAUAAUU | | | | | | | | |
| | UAGU | CUUAGCUUUACUAGUCACU | | | | | | | | |
| | −8_6-6_internal_loop- | GUCGUACAUUGGCCACUCCC | | | | | | | | |
| | symmetric_GAAUUC-UCUUAG | AGU | | | | | | | | |
| | −3_1-1_mismatch_G-A | | | | | | | | | |
| | 0_1-1_mismatch_A-C | | | | | | | | | |
| | 24_6-6_internal_loop- | | | | | | | | | |
| | symmetric_UCAAAG-GACGCC | | | | | | | | | |
| | 40_4-4_bulge-symmetric_GAGU- | | | | | | | | | |
| | CAGA | | | | | | | | | |
| 2541 | −33_4-4_bulge-symmetric_UUCG- | GCCACACAGACCUCCUCAUU | −6 | 28 | 0.064 | 0.932 | 0.928 | 0.965 | 1.722 | 1.669 |
| | ACAU | GAUUGAAAGUAAUUUCAUG | | | | | | | | |
| | −20_4-4_bulge-symmetric_UGGU- | AAUACAUCCACGGCUACUA | | | | | | | | |
| | UAGU | CUGUCCUUUACUAGUCACU | | | | | | | | |
| | −6_6-6_internal_loop- | GUCGUACAUUGGCCACUCCC | | | | | | | | |
| | symmetric_AUUCAU-CUACUG | AGU | | | | | | | | |
| | 0_1-1_mismatch_A-C | | | | | | | | | |
| | 18_2-2_bulge-symmetric_GG-AA | | | | | | | | | |
| | 28_6-6_internal_loop- | | | | | | | | | |
| | symmetric_AGGCCA-CAUUGA | | | | | | | | | |
| | 40_4-4_bulge-symmetric_GAGU- | | | | | | | | | |
| | CAGA | | | | | | | | | |
| 2542 | −33_4-4_bulge-symmetric_UUCG- | GCCACACAGACCUCCUUGUU | −8 | 26 | 0.354 | 0.773 | 0.794 | 1.225 | 1.538 | 1.543 |
| | ACAU | AUCGGAAAGUCCUUUCAUG | | | | | | | | |
| | −20_4-4_bulge-symmetric_UGGU- | AAUACAACCACGGCUAAUA | | | | | | | | |
| | UAGU | CCCGGCUUUACUAGUCACU | | | | | | | | |
| | −8_6-6_internal_loop- | GUCGUACAUUGGCCACUCCC | | | | | | | | |
| | symmetric_GAAUUC-ACCCGG | AGU | | | | | | | | |
| | 0_1-1_mismatch_A-C | | | | | | | | | |
| | 4_1-1_mismatch_A-A | | | | | | | | | |
| | 26_6-6_internal_loop- | | | | | | | | | |
| | symmetric_AAAGGC-UUAUCG | | | | | | | | | |
| | 40_4-4_bulge-symmetric_GAGU- | | | | | | | | | |
| | CAGA | | | | | | | | | |
| 2543 | −33_4-4_bulge-symmetric_UUCG- | GCCACACAGACCUCGACUAA | −6 | 30 | 0.279 | 0.929 | 0.917 | 1.146 | 1.697 | 1.692 |
| | ACAU | CUUUGAAAGUCCUUUCAUG | | | | | | | | |
| | −20_4-4_bulge-symmetric_UGGU- | AAUAGCUCCACGGCUACUA | | | | | | | | |
| | UAGU | GUGUCCUUUACUAGUCACU | | | | | | | | |
| | −6_6-6_internal_loop- | GUCGUACAUUGGCCACUCCC | | | | | | | | |
| | symmetric_AUUCAU-CUAGUG | AGU | | | | | | | | |
| | 0_1-1_mismatch_A-C | | | | | | | | | |
| | 5_2-2_bulge-symmetric_UG-GC | | | | | | | | | |
| | 30_6-6_internal_loop- | | | | | | | | | |
| | symmetric_GCCAAG-GACUAA | | | | | | | | | |
| | 40_4-4_bulge-symmetric_GAGU- | | | | | | | | | |
| | CAGA | | | | | | | | | |
| 2544 | −33_4-4_bulge-symmetric_UUCG- | GCCACACAGACCUCCUGUUA | −12 | 28 | 0.079 | 0.883 | 0.84 | 0.96 | 1.57 | 1.517 |
| | ACAU | UGUUGAAAGUAGUUUCAUG | | | | | | | | |
| | −20_4-4_bulge-symmetric_UGGU- | AAUACAGCCACGACUAAUG | | | | | | | | |
| | UAGU | AAUGCGACGACUAGUCACU | | | | | | | | |
| | −12_6-6_internal_loop- | GUCGUACAUUGGCCACUCCC | | | | | | | | |
| | symmetric_AAAGGA-GCGACG | AGU | | | | | | | | |
| | −2_1-1_mismatch_C-A | | | | | | | | | |
| | 0_1-1_mismatch_A-C | | | | | | | | | |
| | 4_1-1_mismatch_A-G | | | | | | | | | |
| | 18_2-2_bulge-symmetric_GG-AG | | | | | | | | | |
| | 28_6-6_internal_loop- | | | | | | | | | |
| | symmetric_AGGCCA-GUUAUG | | | | | | | | | |
| | 40_4-4_bulge-symmetric_GAGU- | | | | | | | | | |
| | CAGA | | | | | | | | | |
| 2545 | −33_4-4_bulge-symmetric_UUCG- | GCCACACAGACCGGACAAGC | −6 | 32 | 0.027 | 0.684 | 0.789 | 0.927 | 1.489 | 1.514 |
| | ACAU | CUUUGAAAGUAACUUCAUA | | | | | | | | |
| | −20_4-4_bulge-symmetric_UGGU- | UCAUCCACGGCUACGUUUC | | | | | | | | |
| | UAGU | UCCUUUACUAGUCACUGUC | | | | | | | | |
| | −6_6-6_internal_loop- | GUACAUUGGCCACUCCCAG | | | | | | | | |
| | symmetric_AUUCAU-CGUUUC | U | | | | | | | | |
| | 0_1-1_mismatch_A-C | | | | | | | | | |
| | 7_5-2_internal_loop- | | | | | | | | | |
| | asymmetric_UAUUC-AU | | | | | | | | | |
| | 17_3-3_bulge-symmetric_AGG-AAC | | | | | | | | | |
| | 32_6-6_internal_loop- | | | | | | | | | |
| | symmetric_CAAGGA-GGACAA | | | | | | | | | |
| | 40_4-4_bulge-symmetric_GAGU- | | | | | | | | | |
| | CAGA | | | | | | | | | |
| 2546 | −33_4-4_bulge-symmetric_UUCG- | GCCACACAGACCUCCUCCAG | −8 | 28 | 0.039 | 0.885 | 0.888 | 0.913 | 1.657 | 1.645 |
| | ACAU | AGUUGAAAGUCCUUUUAUG | | | | | | | | |
| | −20_4-4_bulge-symmetric_UGGU- | AAUACAUCCACGGCUAAUU | | | | | | | | |
| | UAGU | CCUAGCUUUACUAGUCACU | | | | | | | | |
| | −8_6-6_internal_loop- | GUCGUACAUUGGCCACUCCC | | | | | | | | |
| | symmetric_GAAUUC-UCCUAG | AGU | | | | | | | | |
| | 0_1-1_mismatch_A-C | | | | | | | | | |
| | 14_1-1_wobble_G-U | | | | | | | | | |
| | 28_6-6_internal_loop- | | | | | | | | | |
| | symmetric_AGGCCA-CCAGAG | | | | | | | | | |
| | 40_4-4_bulge-symmetric_GAGU- | | | | | | | | | |
| | CAGA | | | | | | | | | |
| 2547 | −33_4-4_bulge-symmetric_UUCG- | GCCACACAGACCUCGACCGA | −8 | 30 | 0.1 | 0.474 | 0.611 | 1.046 | 0.965 | 1.1 |
| | ACAU | CUUUGAAAGUCCUUUCAUG | | | | | | | | |
| | −20_4-4_bulge-symmetric_UGGU- | CCUACAUCCACGGCUAAUCC | | | | | | | | |
| | UAGU | CGAACUUUACUAGUCACUG | | | | | | | | |
| | −8_6-6_internal_loop- | UCGUACAUUGGCCACUCCCA | | | | | | | | |
| | symmetric_GAAUUC-CCCGAA | GU | | | | | | | | |
| | 0_1-1_mismatch_A-C | | | | | | | | | |
| | 9_2-2_bulge-symmetric_UU-CC | | | | | | | | | |
| | 30_6-6_internal_loop- | | | | | | | | | |
| | symmetric_GCCAAG-GACCGA | | | | | | | | | |
| | 40_4-4_bulge-symmetric_GAGU- | | | | | | | | | |
| | CAGA | | | | | | | | | |
| 2548 | −33_4-4_bulge-symmetric_UUCG- | GCCACACAGACCUCACCCCA | −18 | 30 | 0.18 | 0.94 | 0.947 | 1.003 | 1.418 | 1.509 |
| | ACAU | CUUUGAAAGUCCUUUCAUU | | | | | | | | |
| | −18_6-6_internal_loop- | AAUACUUCCACGGCUAAUG | | | | | | | | |
| | symmetric_UGGUGU-UUGUGU | AAUUCCUUUUUGUGUCACU | | | | | | | | |
| | 0_1-1_mismatch_A-C | GUCGUACAUUGGCCACUCCC | | | | | | | | |
| | 5_1-1_mismatch_U-U | AGU | | | | | | | | |
| | 11_1-1_mismatch_C-U | | | | | | | | | |
| | 30_6-6_internal_loop- | | | | | | | | | |
| | symmetric_GCCAAG-ACCCCA | | | | | | | | | |
| | 40_4-4_bulge-symmetric_GAGU- | | | | | | | | | |
| | CAGA | | | | | | | | | |
| 2549 | −33_4-4_bulge-symmetric_UUCG- | GCCACACAGACCUCGAACGA | −6 | 30 | 0.194 | 0.621 | 0.701 | 1.093 | 1.403 | 1.489 |
| | ACAU | CUUUGAAAGUCCUUUCAUG | | | | | | | | |
| | −20_4-4_bulge-symmetric_UGGU- | AAUAGUUCCACGGCUACCA | | | | | | | | |
| | UAGU | AUGUCCUUUACUAGUCACU | | | | | | | | |
| | −6_6-6_internal_loop- | GUCGUACAUUGGCCACUCCC | | | | | | | | |
| | symmetric_AUUCAU-CCAAUG | AGU | | | | | | | | |
| | 0_1-1_mismatch_A-C | | | | | | | | | |
| | 5_2-2_bulge-symmetric_UG-GU | | | | | | | | | |
| | 30_6-6_internal_loop- | | | | | | | | | |
| | symmetric_GCCAAG-GAACGA | | | | | | | | | |
| | 40_4-4_bulge-symmetric_GAGU- | | | | | | | | | |
| | CAGA | | | | | | | | | |
| 2550 | −33_4-4_bulge-symmetric_UUCG- | GCCACACAGACCUCCUAAUG | −8 | 28 | 0.151 | 0.912 | 0.896 | 1.041 | 1.612 | 1.602 |
| | ACAU | AAUUGAAAGUCCUUUCAUG | | | | | | | | |
| | −20_4-4_bulge-symmetric_UGGU- | UAUACAUCCACGGUUAAUC | | | | | | | | |
| | UAGU | ACCGACUUUACUAGUCACU | | | | | | | | |
| | −8_6-6_internal_loop- | GUCGUACAUUGGCCACUCCC | | | | | | | | |
| | symmetric_GAAUUC-CACCGA | AGU | | | | | | | | |
| | −3_1-1_wobble_G-U | | | | | | | | | |
| | 0_1-1_mismatch_A-C | | | | | | | | | |
| | 10_1-1_mismatch_U-U | | | | | | | | | |
| | 28_6-6_internal_loop- | | | | | | | | | |
| | symmetric_AGGCCA-AAUGAA | | | | | | | | | |
| | 40_4-4_bulge-symmetric_GAGU- | | | | | | | | | |
| | CAGA | | | | | | | | | |
| 2551 | −33_4-4_bulge-symmetric_UUCG- | GCCACACAGACCUCCUGUUA | −18 | 28 | 0.03 | 0.599 | 0.572 | 0.916 | 1.411 | 1.411 |
| | ACAU | UAUUGAAAGUGACGACAUG | | | | | | | | |
| | −18_6-6_internal_loop- | AAUACAUCCACGGCUAAUG | | | | | | | | |
| | symmetric_UGGUGU-UGAAAU | AAUUCCUUUUGAAAUCACU | | | | | | | | |
| | 0_1-1_mismatch_A-C | GUCGUACAUUGGCCACUCCC | | | | | | | | |
| | 15_5-5_internal_loop- | AGU | | | | | | | | |
| | symmetric_AAAGG-GACGA | | | | | | | | | |
| | 28_6-6_internal_loop- | | | | | | | | | |
| | symmetric_AGGCCA-GUUAUA | | | | | | | | | |
| | 40_4-4_bulge-symmetric_GAGU- | | | | | | | | | |
| | CAGA | | | | | | | | | |
| 2552 | −33_4-4_bulge-symmetric_UUCG- | GCCACACAGACCUCCUUGUU | −20 | 26 | 0.03 | 0.761 | 0.855 | 0.94 | 1.577 | 1.544 |
| | ACAU | AGCAGAAAGUAUCUUCAUA | | | | | | | | |
| | −20_6-6_internal_loop- | AAUAGAUCCACGGCUAAUG | | | | | | | | |
| | symmetric_UGUGGU-UAGAAU | AAUUCCUUUACUAGAAUCU | | | | | | | | |
| | 0_1-1_mismatch_A-C | GUCGUACAUUGGCCACUCCC | | | | | | | | |
| | 6_1-1_mismatch_G-G | AGU | | | | | | | | |
| | 11_1-1_mismatch_C-A | | | | | | | | | |
| | 17_3-3_bulge-symmetric_AGG-AUC | | | | | | | | | |
| | 26_6-6_internal_loop- | | | | | | | | | |
| | symmetric_AAAGGC-UUAGCA | | | | | | | | | |
| | 40_4-4_bulge-symmetric_GAGU- | | | | | | | | | |
| | CAGA | | | | | | | | | |
| 2553 | −33_4-4_bulge-symmetric_UUCG- | GCCACACAGACCUCCUACAU | −6 | 28 | 0.049 | 0.834 | 0.842 | 0.957 | 1.639 | 1.617 |
| | ACAU | UCUUGAAAGUAGUUUCGUG | | | | | | | | |
| | −20_4-4_bulge-symmetric_UGGU- | AAUACAUCCACGGCUAUUC | | | | | | | | |
| | UAGU | ACCUCCUUUACUAGUCACU | | | | | | | | |
| | −6_6-6_internal_loop- | GUCGUACAUUGGCCACUCCC | | | | | | | | |
| | symmetric_AUUCAU-UUCACC | AGU | | | | | | | | |
| | 0_1-1_mismatch_A-C | | | | | | | | | |
| | 13_1-1_wobble_U-G | | | | | | | | | |
| | 18_2-2_bulge-symmetric_GG-AG | | | | | | | | | |
| | 28_6-6_internal_loop- | | | | | | | | | |
| | symmetric_AGGCCA-ACAUUC | | | | | | | | | |
| | 40_4-4_bulge-symmetric_GAGU- | | | | | | | | | |
| | CAGA | | | | | | | | | |
| 2554 | −33_4-4_bulge-symmetric_UUCG- | GCCACACAGACCAGAGCUGC | −18 | 32 | 0.049 | 0.932 | 0.89 | 0.849 | 1.287 | 1.242 |
| | ACAU | CUUUGAAAGUCCUUUGUUG | | | | | | | | |
| | −18_6-6_internal_loop- | AAUAGAUCCACGGCUAAUG | | | | | | | | |
| | symmetric_UGGUGU-UGGUAU | AAUUCCUUUUGGUAUCACU | | | | | | | | |
| | 0_1-1_mismatch_A-C | GUCGUACAUUGGCCACUCCC | | | | | | | | |
| | 6_1-1_mismatch_G-G | AGU | | | | | | | | |
| | 13_2-2_bulge-symmetric_UG-GU | | | | | | | | | |
| | 32_6-6_internal_loop- | | | | | | | | | |
| | symmetric_CAAGGA-AGAGCU | | | | | | | | | |
| | 40_4-4_bulge-symmetric_GAGU- | | | | | | | | | |
| | CAGA | | | | | | | | | |
| 2555 | −33_4-4_bulge-symmetric_UUCG- | GCCACAUGUAUAUCCUUGG | −6 | 38 | 0.235 | 0.916 | 0.892 | 0.739 | 0.799 | 0.818 |
| | ACAU | CCUUUGAAAGUCCAUUCAU | | | | | | | | |
| | −20_4-4_bulge-symmetric_UGGU- | GAAUACAUCCACGGCUACG | | | | | | | | |
| | UAGU | UCGGUCCUUUACUAGUCAC | | | | | | | | |
| | −6_6-6_internal_loop- | UGUCGUACAUUGGCCACUC | | | | | | | | |
| | symmetric_AUUCAU-CGUCGG | CCAGU | | | | | | | | |
| | 0_1-1_mismatch_A-C | | | | | | | | | |
| | 17_1-1_mismatch_A-A | | | | | | | | | |
| | 38_6-6_internal_loop- | | | | | | | | | |
| | symmetric_GGGAGU-UGUAUA | | | | | | | | | |
| 2556 | −33_4-4_bulge-symmetric_UUCG- | GCCACACAGACCGGAGUUG | −18 | 32 | 0.198 | 0.941 | 0.916 | 0.904 | 1.135 | 1.238 |
| | ACAU | CCUUUGAAAGUCCUUUCAU | | | | | | | | |
| | −18_6-6_internal_loop- | GAAUACACACACGGCAAUG | | | | | | | | |
| | symmetric_UGGUGU-CUCGUU | AAUUCCUUUCUCGUUCACU | | | | | | | | |
| | −4_1-0_bulge-asymmetric_A- | GUCGUACAUUGGCCACUCCC | | | | | | | | |
| | 0_1-1_mismatch_A-C | AGU | | | | | | | | |
| | 3_2-2_bulge-symmetric_GA-CA | | | | | | | | | |
| | 32_6-6_internal_loop- | | | | | | | | | |
| | symmetric_CAAGGA-GGAGUU | | | | | | | | | |
| | 40_4-4_bulge-symmetric_GAGU- | | | | | | | | | |
| | CAGA | | | | | | | | | |
| 2557 | −33_4-4_bulge-symmetric_UUCG- | GCCACACAGACCUCCUAUUG | −12 | 28 | 0.079 | 0.883 | 0.883 | 0.942 | 1.284 | 1.283 |
| | ACAU | ACUUGAAAGUCCAUUCAUG | | | | | | | | |
| | −20_4-4_bulge-symmetric_UGGU- | AAUACAUCCACGGUUUAUG | | | | | | | | |
| | UAGU | AAUGGCAUGACUAGUCACU | | | | | | | | |
| | −12_6-6_internal_loop- | GUCGUACAUUGGCCACUCCC | | | | | | | | |
| | symmetric_AAAGGA-GGCAUG | AGU | | | | | | | | |
| | −5_1-1_mismatch_U-U | | | | | | | | | |
| | −3_1-1_wobble_G-U | | | | | | | | | |
| | 0_1-1_mismatch_A-C | | | | | | | | | |
| | 17_1-1_mismatch_A-A | | | | | | | | | |
| | 28_6-6_internal_loop- | | | | | | | | | |
| | symmetric_AGGCCA-AUUGAC | | | | | | | | | |
| | 40_4-4_bulge-symmetric_GAGU- | | | | | | | | | |
| | CAGA | | | | | | | | | |
| 2558 | −33_4-4_bulge-symmetric_UUCG- | GCCACACAGACCUCCUUGUA | −8 | 26 | 0.02 | 0.758 | 0.771 | 0.926 | 1.581 | 1.552 |
| | ACAU | UGUGGAAAGUCCUUUGCUG | | | | | | | | |
| | −20_4-4_bulge-symmetric_UGGU- | AAUACAUCCACGUGUAAUC | | | | | | | | |
| | UAGU | UCUAACUUUACUAGUCACU | | | | | | | | |
| | −8_6-6_internal_loop- | GUCGUACAUUGGCCACUCCC | | | | | | | | |
| | symmetric_GAAUUC-CUCUAA | AGU | | | | | | | | |
| | −2_2-2_bulge-symmetric_GC-UG | | | | | | | | | |
| | 0_1-1_mismatch_A-C | | | | | | | | | |
| | 13_2-2_bulge-symmetric_UG-GC | | | | | | | | | |
| | 26_6-6_internal_loop- | | | | | | | | | |
| | symmetric_AAAGGC-UAUGUG | | | | | | | | | |
| | 40_4-4_bulge-symmetric_GAGU- | | | | | | | | | |
| | CAGA | | | | | | | | | |
| 2559 | −33_4-4_bulge-symmetric_UUCG- | GCCACACAGACCUCCUUGGC | −8 | 22 | 0.047 | 0.749 | 0.768 | 0.935 | 1.389 | 1.324 |
| | ACAU | CUGGACUUGUCCUUUCACU | | | | | | | | |
| | −20_4-4_bulge-symmetric_UGGU- | AAUACAUGCACGGCUAAUA | | | | | | | | |
| | UAGU | CUAUACUUUACUAGUCACU | | | | | | | | |
| | −8_6-6_internal_loop- | GUCGUACAUUGGCCACUCCC | | | | | | | | |
| | symmetric_GAAUUC-ACUAUA | AGU | | | | | | | | |
| | 0_1-1_mismatch_A-C | | | | | | | | | |
| | 3_1-1_mismatch_G-G | | | | | | | | | |
| | 11_2-2_bulge-symmetric_CA-CU | | | | | | | | | |
| | 22_6-6_internal_loop- | | | | | | | | | |
| | symmetric_UUUCAA-GGACUU | | | | | | | | | |
| | 40_4-4_bulge-symmetric_GAGU- | | | | | | | | | |
| | CAGA | | | | | | | | | |
| 2560 | −33_4-4_bulge-symmetric_UUCG- | GCCACACAGACCAAGCGCGC | −8 | 32 | 0.072 | 0.918 | 0.907 | 0.939 | 1.596 | 1.529 |
| | ACAU | CUUUGAAAGUCCAUUCAUU | | | | | | | | |
| | −20_4-4_bulge-symmetric_UGGU- | AAUAACUCCACGGCUAAUU | | | | | | | | |
| | UAGU | UCUUACUUUACUAGUCACU | | | | | | | | |
| | −8_6-6_internal_loop- | GUCGUACAUUGGCCACUCCC | | | | | | | | |
| | symmetric_GAAUUC-UUCUUA | AGU | | | | | | | | |
| | 0_1-1_mismatch_A-C | | | | | | | | | |
| | 5_2-2_bulge-symmetric_UG-AC | | | | | | | | | |
| | 11_1-1_mismatch_C-U | | | | | | | | | |
| | 17_1-1_mismatch_A-A | | | | | | | | | |
| | 32_6-6_internal_loop- | | | | | | | | | |
| | symmetric_CAAGGA-AAGCGC | | | | | | | | | |
| | 40_4-4_bulge-symmetric_GAGU- | | | | | | | | | |
| | CAGA | | | | | | | | | |
| 2561 | −33_4-4_bulge-symmetric_UUCG- | GCCACACAGACCAAGGUCGC | −6 | 32 | 0.044 | 0.851 | 0.883 | 0.911 | 1.462 | 1.451 |
| | ACAU | CUUUGAAAGUGUUUCAAGA | | | | | | | | |
| | −20_4-4_bulge-symmetric_UGGU- | AUACCUCCACGGCUACGUA | | | | | | | | |
| | UAGU | GAUCCUUUACUAGUCACUG | | | | | | | | |
| | −6_6-6_internal_loop- | UCGUACAUUGGCCACUCCCA | | | | | | | | |
| | symmetric_AUUCAU-CGUAGA | GU | | | | | | | | |
| | 0_1-1_mismatch_A-C | | | | | | | | | |
| | 5_1-1_mismatch_U-C | | | | | | | | | |
| | 12_1-1_mismatch_A-A | | | | | | | | | |
| | 18_2-1_bulge-asymmetric_GG-G | | | | | | | | | |
| | 32_6-6_internal_loop- | | | | | | | | | |
| | symmetric_CAAGGA-AAGGUC | | | | | | | | | |
| | 40_4-4_bulge-symmetric_GAGU- | | | | | | | | | |
| | CAGA | | | | | | | | | |
| 2562 | −33_4-4_bulge-symmetric_UUCG- | GCCACACAGACCUCCUUGGC | −20 | 24 | 0.275 | 0.923 | 0.931 | 1.129 | 1.67 | 1.658 |
| | ACAU | AGGCUCAAGUCCUUUCAUG | | | | | | | | |
| | −20_6-6_internal_loop- | AAUACCCCCACGGCUAAUG | | | | | | | | |
| | symmetric_UGUGGU-UUUUGU | AAUUCCUUUACUUUUGUCU | | | | | | | | |
| | 0_1-1_mismatch_A-C | GUCGUACAUUGGCCACUCCC | | | | | | | | |
| | 4_2-2_bulge-symmetric_AU-CC | AGU | | | | | | | | |
| | 24_6-6_internal_loop- | | | | | | | | | |
| | symmetric_UCAAAG-AGGCUC | | | | | | | | | |
| | 40_4-4_bulge-symmetric_GAGU- | | | | | | | | | |
| | CAGA | | | | | | | | | |
| 2563 | −33_4-4_bulge-symmetric_UUCG- | GCCACACAGACCUCCUUGUA | −20 | 26 | 0.085 | 0.926 | 0.925 | 0.916 | 1.678 | 1.622 |
| | ACAU | AAAGGAAAGUCCUUUCAUG | | | | | | | | |
| | −20_6-6_internal_loop- | AAUACAUCCACGGGUUGAA | | | | | | | | |
| | symmetric_UGUGGU-UGGGGU | UUCCUUUACUGGGGUCUGU | | | | | | | | |
| | −3_4-2_bulge-asymmetric_UUAG-GU | CGUACAUUGGCCACUCCCAG | | | | | | | | |
| | 0_1-1_mismatch_A-C | U | | | | | | | | |
| | 26_6-6_internal_loop- | | | | | | | | | |
| | symmetric_AAAGGC-UAAAAG | | | | | | | | | |
| | 40_4-4_bulge-symmetric_GAGU- | | | | | | | | | |
| | CAGA | | | | | | | | | |
| 2564 | −33_4-4_bulge-symmetric_UUCG- | GCCACACAGACCUCCUUGAA | −8 | 26 | 0.13 | 0.867 | 0.845 | 0.988 | 1.626 | 1.564 |
| | ACAU | GACAGAAAGUUCUUUCAUG | | | | | | | | |
| | −20_4-4_bulge-symmetric_UGGU- | AAUACAUCCACGGCUAAUU | | | | | | | | |
| | UAGU | ACGUACUUUACUAGUCACU | | | | | | | | |
| | −8_6-6_internal_loop- | GUCGUACAUUGGCCACUCCC | | | | | | | | |
| | symmetric_GAAUUC-UACGUA | AGU | | | | | | | | |
| | 0_1-1_mismatch_A-C | | | | | | | | | |
| | 19_1-1_wobble_G-U | | | | | | | | | |
| | 26_6-6_internal_loop- | | | | | | | | | |
| | symmetric_AAAGGC-AAGACA | | | | | | | | | |
| | 40_4-4_bulge-symmetric_GAGU- | | | | | | | | | |
| | CAGA | | | | | | | | | |
| 2565 | −33_4-4_bulge-symmetric_UUCG- | GCCACACAGACCUCCUAACA | −6 | 28 | 0.131 | 0.53 | 0.7 | 1.049 | 1.384 | 1.521 |
| | ACAU | GGUUGAAAGUCCUUUCAUC | | | | | | | | |
| | −20_4-4_bulge-symmetric_UGGU- | UUUACUUCCACGGCUACCG | | | | | | | | |
| | UAGU | UAGUCCUUUACUAGUCACU | | | | | | | | |
| | −6_6-6_internal_loop- | GUCGUACAUUGGCCACUCCC | | | | | | | | |
| | symmetric_AUUCAU-CCGUAG | AGU | | | | | | | | |
| | 0_1-1_mismatch_A-C | | | | | | | | | |
| | 5_1-1_mismatch_U-U | | | | | | | | | |
| | 9_3-3_bulge-symmetric_UUC-CUU | | | | | | | | | |
| | 28_6-6_internal_loop- | | | | | | | | | |
| | symmetric_AGGCCA-AACAGG | | | | | | | | | |
| | 40_4-4_bulge-symmetric_GAGU- | | | | | | | | | |
| | CAGA | | | | | | | | | |
| 2566 | −33_4-4_bulge-symmetric_UUCG- | GCCACACAGACCUCCUUGGC | −12 | 24 | 0.23 | 0.928 | 0.905 | 1.022 | 1.552 | 1.474 |
| | ACAU | AAUAAUAAGUCCUUUCAUG | | | | | | | | |
| | −20_4-4_bulge-symmetric_UGGU- | AAUACAUCCACGGCUAAUA | | | | | | | | |
| | UAGU | AAUAGGACGACUAGUCACU | | | | | | | | |
| | −12_6-6_internal_loop- | GUCGUACAUUGGCCACUCCC | | | | | | | | |
| | symmetric_AAAGGA-AGGACG | AGU | | | | | | | | |
| | −8_1-1_mismatch_C-A | | | | | | | | | |
| | 0_1-1_mismatch_A-C | | | | | | | | | |
| | 24_6-6_internal_loop- | | | | | | | | | |
| | symmetric_UCAAAG-AAUAAU | | | | | | | | | |
| | 40_4-4_bulge-symmetric_GAGU- | | | | | | | | | |
| | CAGA | | | | | | | | | |
| 2567 | −33_4-4_bulge-symmetric_UUCG- | GCCACACAGACCUCCUAUUU | −8 | 28 | 0.053 | 0.624 | 0.625 | 0.945 | 1.428 | 1.423 |
| | ACAU | ACUUGAAAGUCCUGUCAUG | | | | | | | | |
| | −20_4-4_bulge-symmetric_UGGU- | AAUACAUCCACGGCGGUAA | | | | | | | | |
| | UAGU | UCCGGGGCUUUACUAGUCA | | | | | | | | |
| | −8_6-6_internal_loop- | CUGUCGUACAUUGGCCACU | | | | | | | | |
| | symmetric_GAAUUC-CCGGGG | CCCAGU | | | | | | | | |
| | −3_0-2_bulge-asymmetric_-GG | | | | | | | | | |
| | 0_1-1_mismatch_A-C | | | | | | | | | |
| | 16_1-1_mismatch_A-G | | | | | | | | | |
| | 28_6-6_internal_loop- | | | | | | | | | |
| | symmetric_AGGCCA-AUUUAC | | | | | | | | | |
| | 40_4-4_bulge-symmetric_GAGU- | | | | | | | | | |
| | CAGA | | | | | | | | | |
| 2568 | −33_4-4_bulge-symmetric_UUCG- | GCCACACAGACCUCCUCCAU | −18 | 28 | 0.046 | 0.921 | 0.915 | 0.939 | 1.735 | 1.721 |
| | ACAU | UCUUGAAAGUCCUUUUAUG | | | | | | | | |
| | −18_6-6_internal_loop- | AAUAGAUCCACGGCAAAUG | | | | | | | | |
| | symmetric_UGGUGU-UAGUGU | AAUUCCUUUUAGUGUCACU | | | | | | | | |
| | −4_1-1_mismatch_A-A | GUCGUACAUUGGCCACUCCC | | | | | | | | |
| | 0_1-1_mismatch_A-C | AGU | | | | | | | | |
| | 6_1-1_mismatch_G-G | | | | | | | | | |
| | 14_1-1_wobble_G-U | | | | | | | | | |
| | 28_6-6_internal_loop- | | | | | | | | | |
| | symmetric_AGGCCA-CCAUUC | | | | | | | | | |
| | 40_4-4_bulge-symmetric_GAGU- | | | | | | | | | |
| | CAGA | | | | | | | | | |
| 2569 | −33_4-4_bulge-symmetric_UUCG- | GCCACAUGAAAGUCCUUGG | −10 | 38 | 0.147 | 0.892 | 0.88 | 0.586 | 0.702 | 0.759 |
| | ACAU | CCUUUGAAAGUCCUUUGAU | | | | | | | | |
| | −20_4-4_bulge-symmetric_UGGU- | GAAUACAUCCACGGCUACG | | | | | | | | |
| | UAGU | CGACACAGAUUACUAGUCA | | | | | | | | |
| | −10_6-6_internal_loop- | CUGUCGUACAUUGGCCACU | | | | | | | | |
| | symmetric_AGGAAU-CACAGA | CCCAGU | | | | | | | | |
| | −6_2-3_bulge-asymmetric_AU-CGC | | | | | | | | | |
| | 0_1-1_mismatch_A-C | | | | | | | | | |
| | 14_1-1_mismatch_G-G | | | | | | | | | |
| | 38_6-6_internal_loop- | | | | | | | | | |
| | symmetric_GGGAGU-UGAAAG | | | | | | | | | |
| 2570 | −33_4-4_bulge-symmetric_UUCG- | GCCACACAGACCUCCUAACA | −18 | 28 | 0.044 | 0.908 | 0.907 | 0.947 | 1.706 | 1.726 |
| | ACAU | GAUUGAAAGUUAUUCAUGA | | | | | | | | |
| | −18_6-6_internal_loop- | AUACAUCCACGGCUAAUGA | | | | | | | | |
| | symmetric_UGGUGU-UGAAAU | AUUCCUUUUGAAAUCACUG | | | | | | | | |
| | 0_1-1_mismatch_A-C | UCGUACAUUGGCCACUCCCA | | | | | | | | |
| | 17_2-1_bulge-asymmetric_AG-A | GU | | | | | | | | |
| | 19_1-1_wobble_G-U | | | | | | | | | |
| | 28_6-6_internal_loop- | | | | | | | | | |
| | symmetric_AGGCCA-AACAGA | | | | | | | | | |
| | 40_4-4_bulge-symmetric_GAGU- | | | | | | | | | |
| | CAGA | | | | | | | | | |
| 2571 | −33_4-4_bulge-symmetric_UUCG- | GCCACAUGGUUAUCCUUGG | −6 | 38 | 0.021 | 0.542 | 0.647 | 0.9 | 1.298 | 1.376 |
| | ACAU | CCUUUGAAAGUCCUGGUGC | | | | | | | | |
| | −20_4-4_bulge-symmetric_UGGU- | UUGAAUCAAUCCACGGCUA | | | | | | | | |
| | UAGU | CGACAGUCCUUUACUAGUC | | | | | | | | |
| | −6_6-6_internal_loop- | ACUGUCGUACAUUGGCCAC | | | | | | | | |
| | symmetric_AUUCAU-CGACAG | UCCCAGU | | | | | | | | |
| | 0_1-1_mismatch_A-C | | | | | | | | | |
| | 6_2-2_bulge-symmetric_GU-CA | | | | | | | | | |
| | 13_1-1_mismatch_U-U | | | | | | | | | |
| | 15_2-4_bulge-asymmetric_AA-GGUG | | | | | | | | | |
| | 38_6-6_internal_loop- | | | | | | | | | |
| | symmetric_GGGAGU-UGGUUA | | | | | | | | | |
| 2572 | −33_4-4_bulge-symmetric_UUCG- | GCCACAUGGUAGUCCUUGG | −18 | 38 | 0.177 | 0.922 | 0.924 | 0.476 | 0.715 | 0.738 |
| | ACAU | CCUUUGAAAGUCCUUUCAU | | | | | | | | |
| | −18_6-6_internal_loop- | GAAUACAUUCACGGCUCAU | | | | | | | | |
| | symmetric_UGGUGU-UUUGAU | GAAUUCCUUUUUUGAUCAC | | | | | | | | |
| | −5_1-1_mismatch_U-C | UGUCGUACAUUGGCCACUC | | | | | | | | |
| | 0_1-1_mismatch_A-C | CCAGU | | | | | | | | |
| | 3_1-1_wobble_G-U | | | | | | | | | |
| | 38_6-6_internal_loop- | | | | | | | | | |
| | symmetric_GGGAGU-UGGUAG | | | | | | | | | |
| 2573 | −33_4-4_bulge-symmetric_UUCG- | GCCACACAGACCUCCUGUUU | −6 | 28 | 0.032 | 0.733 | 0.762 | 0.924 | 1.516 | 1.519 |
| | ACAU | UAUUGAAAGUCCUUGCAUG | | | | | | | | |
| | −20_4-4_bulge-symmetric_UGGU- | GAUACUCCACGGCUACGAU | | | | | | | | |
| | UAGU | AGUCCUUUACUAGUCACUG | | | | | | | | |
| | −6_6-6_internal_loop- | UCGUACAUUGGCCACUCCCA | | | | | | | | |
| | symmetric_AUUCAU-CGAUAG | GU | | | | | | | | |
| | 0_1-1_mismatch_A-C | | | | | | | | | |
| | 5_1-0_bulge-asymmetric_U- | | | | | | | | | |
| | 10_1-1_wobble_U-G | | | | | | | | | |
| | 15_1-1_mismatch_A-G | | | | | | | | | |
| | 28_6-6_internal_loop- | | | | | | | | | |
| | symmetric_AGGCCA-GUUUUA | | | | | | | | | |
| | 40_4-4_bulge-symmetric_GAGU- | | | | | | | | | |
| | CAGA | | | | | | | | | |
| 2574 | −33_4-4_bulge-symmetric_UUCG- | GCCACACAGACCUCCUUGUA | −6 | 26 | 0.035 | 0.836 | 0.886 | 0.952 | 1.664 | 1.708 |
| | ACAU | GACGGAAAGUGGUUUCAUU | | | | | | | | |
| | −20_4-4_bulge-symmetric_UGGU- | CAUAGAUCCACGGCUACGA | | | | | | | | |
| | UAGU | CGCUCCUUUACUAGUCACU | | | | | | | | |
| | −6_6-6_internal_loop- | GUCGUACAUUGGCCACUCCC | | | | | | | | |
| | symmetric_AUUCAU-CGACGC | AGU | | | | | | | | |
| | 0_1-1_mismatch_A-C | | | | | | | | | |
| | 6_1-1_mismatch_G-G | | | | | | | | | |
| | 10_2-2_bulge-symmetric_UC-UC | | | | | | | | | |
| | 18_2-2_bulge-symmetric_GG-GG | | | | | | | | | |
| | 26_6-6_internal_loop- | | | | | | | | | |
| | symmetric_AAAGGC-UAGACG | | | | | | | | | |
| | 40_4-4_bulge-symmetric_GAGU- | | | | | | | | | |
| | CAGA | | | | | | | | | |
| 2575 | −33_4-4_bulge-symmetric_UUCG- | GCCACACAGACCUCCUUGUA | −10 | 26 | 0.25 | 0.869 | 0.869 | 1.098 | 1.528 | 1.502 |
| | ACAU | AGUAGAAAGUCCUUUCAUG | | | | | | | | |
| | −20_4-4_bulge-symmetric_UGGU- | AAUACCACCACGGCCAAUG | | | | | | | | |
| | UAGU | ACACGACUUACUAGUCACU | | | | | | | | |
| | −10_6-6_internal_loop- | GUCGUACAUUGGCCACUCCC | | | | | | | | |
| | symmetric_AGGAAU-CACGAC | AGU | | | | | | | | |
| | −4_1-1_mismatch_A-C | | | | | | | | | |
| | 0_1-1_mismatch_A-C | | | | | | | | | |
| | 4_2-2_bulge-symmetric_AU-CA | | | | | | | | | |
| | 26_6-6_internal_loop- | | | | | | | | | |
| | symmetric_AAAGGC-UAAGUA | | | | | | | | | |
| | 40_4-4_bulge-symmetric_GAGU- | | | | | | | | | |
| | CAGA | | | | | | | | | |
| 2576 | −33_4-4_bulge-symmetric_UUCG- | GCCACACAGACCUCCUUGGC | −8 | 24 | 0.44 | 0.925 | 0.921 | 1.218 | 1.684 | 1.581 |
| | ACAU | GGGGUUAAGUCCUUUCAUG | | | | | | | | |
| | −20_4-4_bulge-symmetric_UGGU- | AAUACACCCACGGCUAAUCC | | | | | | | | |
| | UAGU | UUGGCUUUACUAGUCACUG | | | | | | | | |
| | −8_6-6_internal_loop- | UCGUACAUUGGCCACUCCCA | | | | | | | | |
| | symmetric_GAAUUC-CCUUGG | GU | | | | | | | | |
| | 0_1-1_mismatch_A-C | | | | | | | | | |
| | 4_1-1_mismatch_A-C | | | | | | | | | |
| | 24_6-6_internal_loop- | | | | | | | | | |
| | symmetric_UCAAAG-GGGGUU | | | | | | | | | |
| | 40_4-4_bulge-symmetric_GAGU- | | | | | | | | | |
| | CAGA | | | | | | | | | |
| 2577 | −33_4-4_bulge-symmetric_UUCG- | GCCACACAGACCUCCUGAAA | −8 | 28 | 0.171 | 0.812 | 0.78 | 1.017 | 1.497 | 1.487 |
| | ACAU | UGUUGAAAGUCCUUUCAUG | | | | | | | | |
| | −20_4-4_bulge-symmetric_UGGU- | AAUACAUCCACGGGUAAUU | | | | | | | | |
| | UAGU | AUACGCUUUACUAGUCACU | | | | | | | | |
| | −8_6-6_internal_loop- | GUCGUACAUUGGCCACUCCC | | | | | | | | |
| | symmetric_GAAUUC-UAUACG | AGU | | | | | | | | |
| | −3_1-1_mismatch_G-G | | | | | | | | | |
| | 0_1-1_mismatch_A-C | | | | | | | | | |
| | 28_6-6_internal_loop- | | | | | | | | | |
| | symmetric_AGGCCA-GAAAUG | | | | | | | | | |
| | 40_4-4_bulge-symmetric_GAGU- | | | | | | | | | |
| | CAGA | | | | | | | | | |
| 2578 | −33_4-4_bulge-symmetric_UUCG- | GCCACACAGACCUCCUUGUA | −8 | 26 | 0.156 | 0.709 | 0.792 | 1.069 | 1.439 | 1.547 |
| | ACAU | GACCGAAAGUCCUUUCAUG | | | | | | | | |
| | −20_4-4_bulge-symmetric_UGGU- | AAUAUCAUCCAUAGCUAAU | | | | | | | | |
| | UAGU | UGCACACUUUACUAGUCAC | | | | | | | | |
| | −8_6-6_internal_loop- | UGUCGUACAUUGGCCACUC | | | | | | | | |
| | symmetric_GAAUUC-UGCACA | CCAGU | | | | | | | | |
| | −1_1-1_mismatch_C-A | | | | | | | | | |
| | 6_0-1_bulge-asymmetric_-U | | | | | | | | | |
| | 26_6-6_internal_loop- | | | | | | | | | |
| | symmetric_AAAGGC-UAGACC | | | | | | | | | |
| | 40_4-4_bulge-symmetric_GAGU- | | | | | | | | | |
| | CAGA | | | | | | | | | |
| 2579 | −33_4-4_bulge-symmetric_UUCG- | GCCACACAGACCUCGCAAUG | −12 | 30 | 0.309 | 0.887 | 0.897 | 1.151 | 1.594 | 1.615 |
| | ACAU | CUUUGAAAGUCCUUUCAUG | | | | | | | | |
| | −20_4-4_bulge-symmetric_UGGU- | AAUAAAUCCACGGCUAAUG | | | | | | | | |
| | UAGU | AAUGAGACGACUAGUCACU | | | | | | | | |
| | −12_6-6_internal_loop- | GUCGUACAUUGGCCACUCCC | | | | | | | | |
| | symmetric_AAAGGA-GAGACG | AGU | | | | | | | | |
| | 0_1-1_mismatch_A-C | | | | | | | | | |
| | 6_1-1_mismatch_G-A | | | | | | | | | |
| | 30_6-6_internal_loop- | | | | | | | | | |
| | symmetric_GCCAAG-GCAAUG | | | | | | | | | |
| | 40_4-4_bulge-symmetric_GAGU- | | | | | | | | | |
| | CAGA | | | | | | | | | |
| 2580 | −33_4-4_bulge-symmetric_UUCG- | GCCACACAGACCUCCUAUCA | −18 | 28 | 0.059 | 0.838 | 0.802 | 0.924 | 1.505 | 1.478 |
| | ACAU | AGUUGAAAGUCCUUUCAGC | | | | | | | | |
| | −18_6-6_internal_loop- | AUACAUCCACGGCUAAUGA | | | | | | | | |
| | symmetric_UGGUGU-UGUUUU | AUUCCUUUUGUUUUCACUG | | | | | | | | |
| | 0_1-1_mismatch_A-C | UCGUACAUUGGCCACUCCCA | | | | | | | | |
| | 10_1-1_mismatch_U-C | GU | | | | | | | | |
| | 12_1-0_bulge-asymmetric_A- | | | | | | | | | |
| | 28_6-6_internal_loop- | | | | | | | | | |
| | symmetric_AGGCCA-AUCAAG | | | | | | | | | |
| | 40_4-4_bulge-symmetric_GAGU- | | | | | | | | | |
| | CAGA | | | | | | | | | |
| 2581 | −33_4-4_bulge-symmetric_UUCG- | GCCACACAGACCUCCUUGGC | −10 | 24 | 0.221 | 0.778 | 0.783 | 1.084 | 1.511 | 1.523 |
| | ACAU | GGAGCCAAGUCCUUUCAUG | | | | | | | | |
| | −20_4-4_bulge-symmetric_UGGU- | AAUACAACCACGGCGACGG | | | | | | | | |
| | UAGU | ACCAGGGUUACUAGUCACU | | | | | | | | |
| | −10_6-6_internal_loop- | GUCGUACAUUGGCCACUCCC | | | | | | | | |
| | symmetric_AGGAAU-CCAGGG | AGU | | | | | | | | |
| | −4_4-4_bulge-symmetric_AUUA- | | | | | | | | | |
| | GACG | | | | | | | | | |
| | 0_1-1_mismatch_A-C | | | | | | | | | |
| | 4_1-1_mismatch_A-A | | | | | | | | | |
| | 24_6-6_internal_loop- | | | | | | | | | |
| | symmetric_UCAAAG-GGAGCC | | | | | | | | | |
| | 40_4-4_bulge-symmetric_GAGU- | | | | | | | | | |
| | CAGA | | | | | | | | | |
| 2582 | −33_4-4_bulge-symmetric_UUCG- | GCCACACAGACCUCCUCUUA | −6 | 28 | 0.077 | 0.688 | 0.716 | 0.989 | 1.49 | 1.502 |
| | ACAU | UAUUGAAAGUCCUUUCAUG | | | | | | | | |
| | −20_4-4_bulge-symmetric_UGGU- | AAUAUAUCGACGGCUACGA | | | | | | | | |
| | UAGU | CAAUCCUUUACUAGUCACU | | | | | | | | |
| | −6_6-6_internal_loop- | GUCGUACAUUGGCCACUCCC | | | | | | | | |
| | symmetric_AUUCAU-CGACAA | AGU | | | | | | | | |
| | 0_1-1_mismatch_A-C | | | | | | | | | |
| | 2_1-1_mismatch_G-G | | | | | | | | | |
| | 6_1-1_wobble_G-U | | | | | | | | | |
| | 28_6-6_internal_loop- | | | | | | | | | |
| | symmetric_AGGCCA-CUUAUA | | | | | | | | | |
| | 40_4-4_bulge-symmetric_GAGU- | | | | | | | | | |
| | CAGA | | | | | | | | | |
| 2583 | −33_4-4_bulge-symmetric_UUCG- | GCCACACAGACCUCCUUGAA | −8 | 26 | 0.028 | 0.57 | 0.619 | 0.928 | 1.412 | 1.408 |
| | ACAU | UCAAGAAAGUCGGUUCAUG | | | | | | | | |
| | −20_4-4_bulge-symmetric_UGGU- | AAUACUCCCACGGCUAAUU | | | | | | | | |
| | UAGU | AGCGACUUUACUAGUCACU | | | | | | | | |
| | −8_6-6_internal_loop- | GUCGUACAUUGGCCACUCCC | | | | | | | | |
| | symmetric_GAAUUC-UAGCGA | AGU | | | | | | | | |
| | 0_1-1_mismatch_A-C | | | | | | | | | |
| | 4_2-2_bulge-symmetric_AU-UC | | | | | | | | | |
| | 17_2-2_bulge-symmetric_AG-GG | | | | | | | | | |
| | 26_6-6_internal_loop- | | | | | | | | | |
| | symmetric_AAAGGC-AAUCAA | | | | | | | | | |
| | 40_4-4_bulge-symmetric_GAGU- | | | | | | | | | |
| | CAGA | | | | | | | | | |
| 2584 | −33_4-4_bulge-symmetric_UUCG- | GCCACACAGACCUCCUUGUA | −18 | 26 | 0.033 | 0.911 | 0.881 | 0.905 | 1.673 | 1.607 |
| | ACAU | GUAGGAAAGUCGUUUCCAU | | | | | | | | |
| | −18_6-6_internal_loop- | GAAUACAUCCACGGCUAAU | | | | | | | | |
| | symmetric_UGGUGU-UUCGUU | GAAUUCCUUUUUCGUUCAC | | | | | | | | |
| | 0_1-1_mismatch_A-C | UGUCGUACAUUGGCCACUC | | | | | | | | |
| | 14_0-1_bulge-asymmetric_-C | CCAGU | | | | | | | | |
| | 18_1-1_mismatch_G-G | | | | | | | | | |
| | 26_6-6_internal_loop- | | | | | | | | | |
| | symmetric_AAAGGC-UAGUAG | | | | | | | | | |
| | 40_4-4_bulge-symmetric_GAGU- | | | | | | | | | |
| | CAGA | | | | | | | | | |
| 2585 | −33_4-4_bulge-symmetric_UUCG- | GCCACACAGACCUCCUUGAA | −8 | 26 | 0.07 | 0.924 | 0.891 | 0.931 | 1.654 | 1.614 |
| | ACAU | AAUGGAAAGUCCUGUCAUU | | | | | | | | |
| | −20_4-4_bulge-symmetric_UGGU- | AAUACAUCCACGGCUAAUU | | | | | | | | |
| | UAGU | UGGGACUUUACUAGUCACU | | | | | | | | |
| | −8_6-6_internal_loop- | GUCGUACAUUGGCCACUCCC | | | | | | | | |
| | symmetric_GAAUUC-UUGGGA | AGU | | | | | | | | |
| | 0_1-1_mismatch_A-C | | | | | | | | | |
| | 11_1-1_mismatch_C-U | | | | | | | | | |
| | 16_1-1_mismatch_A-G | | | | | | | | | |
| | 26_6-6_internal_loop- | | | | | | | | | |
| | symmetric_AAAGGC-AAAAUG | | | | | | | | | |
| | 40_4-4_bulge-symmetric_GAGU- | | | | | | | | | |
| | CAGA | | | | | | | | | |
| 2586 | −33_4-4_bulge-symmetric_UUCG- | GCCACACAGACCUCCUCAUU | −8 | 28 | 0.182 | 0.92 | 0.929 | 1.015 | 1.623 | 1.583 |
| | ACAU | UGUUGAAAGUCCUCUCAUG | | | | | | | | |
| | −20_4-4_bulge-symmetric_UGGU- | AAUACAUCCACGGCUAAUC | | | | | | | | |
| | UAGU | AUUGGCUUUACUAGUCACU | | | | | | | | |
| | −8_6-6_internal_loop- | GUCGUACAUUGGCCACUCCC | | | | | | | | |
| | symmetric_GAAUUC-CAUUGG | AGU | | | | | | | | |
| | 0_1-1_mismatch_A-C | | | | | | | | | |
| | 16_1-1_mismatch_A-C | | | | | | | | | |
| | 28_6-6_internal_loop- | | | | | | | | | |
| | symmetric_AGGCCA-CAUUUG | | | | | | | | | |
| | 40_4-4_bulge-symmetric_GAGU- | | | | | | | | | |
| | CAGA | | | | | | | | | |
| 2587 | −33_4-4_bulge-symmetric_UUCG- | GCCACACAGACCUCAAGCAG | −8 | 30 | 0.305 | 0.896 | 0.879 | 1.142 | 1.459 | 1.448 |
| | ACAU | CUUUGAAAGUCCUUUCAUG | | | | | | | | |
| | −20_4-4_bulge-symmetric_UGGU- | AAUACCUCCACGGCUAAUCC | | | | | | | | |
| | UAGU | AAGGCUUUACUAGUCACUG | | | | | | | | |
| | −8_6-6_internal_loop- | UCGUACAUUGGCCACUCCCA | | | | | | | | |
| | symmetric_GAAUUC-CCAAGG | GU | | | | | | | | |
| | 0_1-1_mismatch_A-C | | | | | | | | | |
| | 5_1-1_mismatch_U-C | | | | | | | | | |
| | 30_6-6_internal_loop- | | | | | | | | | |
| | symmetric_GCCAAG-AAGCAG | | | | | | | | | |
| | 40_4-4_bulge-symmetric_GAGU- | | | | | | | | | |
| | CAGA | | | | | | | | | |
| 2588 | −33_4-4_bulge-symmetric_UUCG- | GCCACAUGGAAGUCCUUGG | −8 | 38 | 0.174 | 0.919 | 0.939 | 0.615 | 1.187 | 1.061 |
| | ACAU | CCUUUGAAAGUCCUUUCAU | | | | | | | | |
| | −20_4-4_bulge-symmetric_UGGU- | GAAUCGAUCCACGGCUAAU | | | | | | | | |
| | UAGU | CUACGGCUUUACUAGUCAC | | | | | | | | |
| | −8_6-6_internal_loop- | UGUCGUACAUUGGCCACUC | | | | | | | | |
| | symmetric_GAAUUC-CUACGG | CCAGU | | | | | | | | |
| | 0_1-1_mismatch_A-C | | | | | | | | | |
| | 6_2-2_bulge-symmetric_GU-CG | | | | | | | | | |
| | 38_6-6_internal_loop- | | | | | | | | | |
| | symmetric_GGGAGU-UGGAAG | | | | | | | | | |
| 2589 | −33_4-4_bulge-symmetric_UUCG- | GCCACACAGACCUCCUGUUU | −8 | 28 | 0.065 | 0.566 | 0.573 | 0.929 | 1.328 | 1.317 |
| | ACAU | ACUUGAAAGUCCUCUCAUG | | | | | | | | |
| | −20_4-4_bulge-symmetric_UGGU- | AAUACAUCCACGGUAAUUC | | | | | | | | |
| | UAGU | AAGGCUUUACUAGUCACUG | | | | | | | | |
| | −8_6-6_internal_loop- | UCGUACAUUGGCCACUCCCA | | | | | | | | |
| | symmetric_GAAUUC-UCAAGG | GU | | | | | | | | |
| | −3_1-0_bulge-asymmetric_G- | | | | | | | | | |
| | 0_1-1_mismatch_A-C | | | | | | | | | |
| | 16_1-1_mismatch_A-C | | | | | | | | | |
| | 28_6-6_internal_loop- | | | | | | | | | |
| | symmetric_AGGCCA-GUUUAC | | | | | | | | | |
| | 40_4-4_bulge-symmetric_GAGU- | | | | | | | | | |
| | CAGA | | | | | | | | | |
| 2590 | −33_4-4_bulge-symmetric_UUCG- | GCCACAUAGGGAUCCUUGG | −18 | 38 | 0.403 | 0.927 | 0.929 | 0.542 | 0.701 | 0.726 |
| | ACAU | CCUUUGAAAGUCCUUUCAU | | | | | | | | |
| | −18_6-6_internal_loop- | GAAUACAUCCACGGCGAAU | | | | | | | | |
| | symmetric_UGGUGU-UGUUAU | GAAUUCCUUUUGUUAUCAC | | | | | | | | |
| | −4_1-1_mismatch_A-G | UGUCGUACAUUGGCCACUC | | | | | | | | |
| | 0_1-1_mismatch_A-C | CCAGU | | | | | | | | |
| | 38_6-6_internal_loop- | | | | | | | | | |
| | symmetric_GGGAGU-UAGGGA | | | | | | | | | |
| 2591 | −33_4-4_bulge-symmetric_UUCG- | GCCACACAGACCUCAGCCUA | −10 | 30 | 0.33 | 0.93 | 0.916 | 1.196 | 1.435 | 1.472 |
| | ACAU | CUUUGAAAGUCCUUUCAUC | | | | | | | | |
| | −20_4-4_bulge-symmetric_UGGU- | AAUACCACCACGGCUAAUG | | | | | | | | |
| | UAGU | ACAGUACUUACUAGUCACU | | | | | | | | |
| | −10_6-6_internal_loop- | GUCGUACAUUGGCCACUCCC | | | | | | | | |
| | symmetric_AGGAAU-CAGUAC | AGU | | | | | | | | |
| | 0_1-1_mismatch_A-C | | | | | | | | | |
| | 4_2-2_bulge-symmetric_AU-CA | | | | | | | | | |
| | 11_1-1_mismatch_C-C | | | | | | | | | |
| | 30_6-6_internal_loop- | | | | | | | | | |
| | symmetric_GCCAAG-AGCCUA | | | | | | | | | |
| | 40_4-4_bulge-symmetric_GAGU- | | | | | | | | | |
| | CAGA | | | | | | | | | |
| 2592 | −33_4-4_bulge-symmetric_UUCG- | GCCACACAGACCUCCUUGUA | −20 | 26 | 0.023 | 0.927 | 0.923 | 0.91 | 1.696 | 1.697 |
| | ACAU | GGGGGAAAGUCCUUGAAUG | | | | | | | | |
| | −20_6-6_internal_loop- | AAUACAUCCACGGCUAAUG | | | | | | | | |
| | symmetric_UGUGGU-UGUGUU | AAUUCCUUUACUGUGUUCU | | | | | | | | |
| | 0_1-1_mismatch_A-C | GUCGUACAUUGGCCACUCCC | | | | | | | | |
| | 14_2-2_bulge-symmetric_GA-GA | AGU | | | | | | | | |
| | 26_6-6_internal_loop- | | | | | | | | | |
| | symmetric_AAAGGC-UAGGGG | | | | | | | | | |
| | 40_4-4_bulge-symmetric_GAGU- | | | | | | | | | |
| | CAGA | | | | | | | | | |
| 2593 | −33_4-4_bulge-symmetric_UUCG- | GCCACACAGACCUCCUUGUA | −10 | 26 | 0.025 | 0.553 | 0.594 | 0.933 | 1.236 | 1.187 |
| | ACAU | UGGGGAAAGUCCCUUGAUG | | | | | | | | |
| | −20_4-4_bulge-symmetric_UGGU- | ACACAUCCACGGCUAAUGA | | | | | | | | |
| | UAGU | CGCGGAUUACUAGUCACUG | | | | | | | | |
| | −10_6-6_internal_loop- | UCGUACAUUGGCCACUCCCA | | | | | | | | |
| | symmetric_AGGAAU-CGCGGA | GU | | | | | | | | |
| | 0_1-1_mismatch_A-C | | | | | | | | | |
| | 8_2-1_bulge-asymmetric_AU-C | | | | | | | | | |
| | 14_1-1_mismatch_G-G | | | | | | | | | |
| | 17_1-1_mismatch_A-C | | | | | | | | | |
| | 26_6-6_internal_loop- | | | | | | | | | |
| | symmetric_AAAGGC-UAUGGG | | | | | | | | | |
| | 40_4-4_bulge-symmetric_GAGU- | | | | | | | | | |
| | CAGA | | | | | | | | | |
| 2594 | −33_4-4_bulge-symmetric_UUCG- | GCCACACAGACCUCCUACUG | −6 | 28 | 0.034 | 0.752 | 0.783 | 0.939 | 1.404 | 1.366 |
| | ACAU | ACUUGAAAGUCAGUUUCAU | | | | | | | | |
| | −20_4-4_bulge-symmetric_UGGU- | GAAUACAUCCACGGCUAUC | | | | | | | | |
| | UAGU | UAGCUCCUUUACUAGUCAC | | | | | | | | |
| | −6_6-6_internal_loop- | UGUCGUACAUUGGCCACUC | | | | | | | | |
| | symmetric_AUUCAU-UCUAGC | CCAGU | | | | | | | | |
| | 0_1-1_mismatch_A-C | | | | | | | | | |
| | 18_1-2_bulge-asymmetric_G-AG | | | | | | | | | |
| | 28_6-6_internal_loop- | | | | | | | | | |
| | symmetric_AGGCCA-ACUGAC | | | | | | | | | |
| | 40_4-4_bulge-symmetric_GAGU- | | | | | | | | | |
| | CAGA | | | | | | | | | |
| 2595 | −33_4-4_bulge-symmetric_UUCG- | GCCACACAGACCUCCUUGUU | −8 | 26 | 0.047 | 0.76 | 0.742 | 0.949 | 1.587 | 1.526 |
| | ACAU | GAUGGAAAGUCCUUUCAUG | | | | | | | | |
| | −20_4-4_bulge-symmetric_UGGU- | AAUACCCCACGGCUAAUAC | | | | | | | | |
| | UAGU | AGGGCUUUACUAGUCACUG | | | | | | | | |
| | −8_6-6_internal_loop- | UCGUACAUUGGCCACUCCCA | | | | | | | | |
| | symmetric_GAAUUC-ACAGGG | GU | | | | | | | | |
| | 0_1-1_mismatch_A-C | | | | | | | | | |
| | 4_2-1_bulge-asymmetric_AU-C | | | | | | | | | |
| | 26_6-6_internal_loop- | | | | | | | | | |
| | symmetric_AAAGGC-UUGAUG | | | | | | | | | |
| | 40_4-4_bulge-symmetric_GAGU- | | | | | | | | | |
| | CAGA | | | | | | | | | |
| 2596 | −33_4-4_bulge-symmetric_UUCG- | GCCACACAGACCUCCUUGUU | −18 | 26 | 0.296 | 0.959 | 0.942 | 1.135 | 1.726 | 1.664 |
| | ACAU | GAAGGAAAGUCCUUUCAUG | | | | | | | | |
| | −18_6-6_internal_loop- | AAUACUUCCACGGCUAAUG | | | | | | | | |
| | symmetric_UGGUGU-UUUGUU | AAUUCCUUUUUUGUUCACU | | | | | | | | |
| | 0_1-1_mismatch_A-C | GUCGUACAUUGGCCACUCCC | | | | | | | | |
| | 5_1-1_mismatch_U-U | AGU | | | | | | | | |
| | 26_6-6_internal_loop- | | | | | | | | | |
| | symmetric_AAAGGC-UUGAAG | | | | | | | | | |
| | 40_4-4_bulge-symmetric_GAGU- | | | | | | | | | |
| | CAGA | | | | | | | | | |
| 2597 | −33_4-4_bulge-symmetric_UUCG- | GCCACACAGACCUCCUUGUA | −20 | 26 | 0.052 | 0.862 | 0.833 | 0.887 | 1.604 | 1.588 |
| | ACAU | GGACGAAAGUACUUUCAUG | | | | | | | | |
| | −20_6-6_internal_loop- | AAUACAUCCAUAGAUGCUG | | | | | | | | |
| | symmetric_UGUGGU-UAGAAU | AAUUCCUUUACUAGAAUCU | | | | | | | | |
| | −6_1-1_mismatch_U-C | GUCGUACAUUGGCCACUCCC | | | | | | | | |
| | −5_1-1_wobble_U-G | AGU | | | | | | | | |
| | −3_1-1_mismatch_G-A | | | | | | | | | |
| | −1_1-1_mismatch_C-A | | | | | | | | | |
| | 19_1-1_mismatch_G-A | | | | | | | | | |
| | 26_6-6_internal_loop- | | | | | | | | | |
| | symmetric_AAAGGC-UAGGAC | | | | | | | | | |
| | 40_4-4_bulge-symmetric_GAGU- | | | | | | | | | |
| | CAGA | | | | | | | | | |
| 2598 | −33_4-4_bulge-symmetric_UUCG- | GCCACAUAGGGGUCCUUGG | −6 | 38 | 0.116 | 0.92 | 0.929 | 0.604 | 0.796 | 0.786 |
| | ACAU | CCUUUGAAAGUCUUUUCAU | | | | | | | | |
| | −20_4-4_bulge-symmetric_UGGU- | GAAUUCAUCCACGGCUACG | | | | | | | | |
| | UAGU | CUAGUCCUUUACUAGUCAC | | | | | | | | |
| | −6_6-6_internal_loop- | UGUCGUACAUUGGCCACUC | | | | | | | | |
| | symmetric_AUUCAU-CGCUAG | CCAGU | | | | | | | | |
| | 0_1-1_mismatch_A-C | | | | | | | | | |
| | 7_1-1_mismatch_U-U | | | | | | | | | |
| | 18_1-1_wobble_G-U | | | | | | | | | |
| | 38_6-6_internal_loop- | | | | | | | | | |
| | symmetric_GGGAGU-UAGGGG | | | | | | | | | |
| 2599 | −33_4-4_bulge-symmetric_UUCG- | GCCACACAGACCAGGUAUG | −6 | 32 | 0.065 | 0.584 | 0.629 | 0.971 | 1.28 | 1.326 |
| | ACAU | CCUUUGAAAGUCAAUUCAU | | | | | | | | |
| | −20_4-4_bulge-symmetric_UGGU- | GAAUACUUCCACGACUAUA | | | | | | | | |
| | UAGU | AAACUCCUUUACUAGUCAC | | | | | | | | |
| | −6_6-6_internal_loop- | UGUCGUACAUUGGCCACUC | | | | | | | | |
| | symmetric_AUUCAU-UAAAAC | CCAGU | | | | | | | | |
| | −2_1-1_mismatch_C-A | | | | | | | | | |
| | 0_1-1_mismatch_A-C | | | | | | | | | |
| | 5_1-1_mismatch_U-U | | | | | | | | | |
| | 17_2-2_bulge-symmetric_AG-AA | | | | | | | | | |
| | 32_6-6_internal_loop- | | | | | | | | | |
| | symmetric_CAAGGA-AGGUAU | | | | | | | | | |
| | 40_4-4_bulge-symmetric_GAGU- | | | | | | | | | |
| | CAGA | | | | | | | | | |
| 2600 | −33_4-4_bulge-symmetric_UUCG- | GCCACACAGACCUCCUUGAU | −6 | 26 | 0.379 | 0.913 | 0.908 | 1.219 | 1.72 | 1.652 |
| | ACAU | AGGCGAAAGUCCUUUCAUG | | | | | | | | |
| | −20_4-4_bulge-symmetric_UGGU- | AAUAACUCCACGGCUAUCA | | | | | | | | |
| | UAGU | UACUCCUUUACUAGUCACU | | | | | | | | |
| | −6_6-6_internal_loop- | GUCGUACAUUGGCCACUCCC | | | | | | | | |
| | symmetric_AUUCAU-UCAUAC | AGU | | | | | | | | |
| | 0_1-1_mismatch_A-C | | | | | | | | | |
| | 5_2-2_bulge-symmetric_UG-AC | | | | | | | | | |
| | 26_6-6_internal_loop- | | | | | | | | | |
| | symmetric_AAAGGC-AUAGGC | | | | | | | | | |
| | 40_4-4_bulge-symmetric_GAGU- | | | | | | | | | |
| | CAGA | | | | | | | | | |
| 2601 | −33_4-4_bulge-symmetric_UUCG- | GCCACACAGACCUCCUUGUA | −18 | 26 | 0.034 | 0.886 | 0.877 | 0.916 | 1.639 | 1.631 |
| | ACAU | UGAGGAAAGUCCUUUCCAG | | | | | | | | |
| | −18_6-6_internal_loop- | AAUACAUCCACGGCUAAUG | | | | | | | | |
| | symmetric_UGGUGU-UAGUGU | AAUUCCUUUUAGUGUCACU | | | | | | | | |
| | 0_1-1_mismatch_A-C | GUCGUACAUUGGCCACUCCC | | | | | | | | |
| | 12_2-2_bulge-symmetric_AU-CA | AGU | | | | | | | | |
| | 26_6-6_internal_loop- | | | | | | | | | |
| | symmetric_AAAGGC-UAUGAG | | | | | | | | | |
| | 40_4-4_bulge-symmetric_GAGU- | | | | | | | | | |
| | CAGA | | | | | | | | | |
| 2602 | −33_4-4_bulge-symmetric_UUCG- | GCCACACAGACCUCCUGACU | −10 | 28 | 0.122 | 0.771 | 0.771 | 0.838 | 0.915 | 0.925 |
| | ACAU | AAUUGAAAGUCCUUUCAUG | | | | | | | | |
| | −20_4-4_bulge-symmetric_UGGU- | AAUACACGCACGGCUCGUG | | | | | | | | |
| | UAGU | ACACGGAUUACUAGUCACU | | | | | | | | |
| | −10_6-6_internal_loop- | GUCGUACAUUGGCCACUCCC | | | | | | | | |
| | symmetric_AGGAAU-CACGGA | AGU | | | | | | | | |
| | −6_1-1_wobble_U-G | | | | | | | | | |
| | −5_1-1_mismatch_U-C | | | | | | | | | |
| | 0_1-1_mismatch_A-C | | | | | | | | | |
| | 3_2-2_bulge-symmetric_GA-CG | | | | | | | | | |
| | 28_6-6_internal_loop- | | | | | | | | | |
| | symmetric_AGGCCA-GACUAA | | | | | | | | | |
| | 40_4-4_bulge-symmetric_GAGU- | | | | | | | | | |
| | CAGA | | | | | | | | | |
| 2603 | −33_4-4_bulge-symmetric_UUCG- | GCCACACAGACCUCCUGAUA | −8 | 28 | 0.158 | 0.75 | 0.756 | 1.007 | 1.405 | 1.431 |
| | ACAU | GAUUGAAAGUCCUUUCAUG | | | | | | | | |
| | −20_4-4_bulge-symmetric_UGGU- | CCUACAUCCACGGCUAAUA | | | | | | | | |
| | UAGU | UGCGGCUUUACUAGUCACU | | | | | | | | |
| | −8_6-6_internal_loop- | GUCGUACAUUGGCCACUCCC | | | | | | | | |
| | symmetric_GAAUUC-AUGCGG | AGU | | | | | | | | |
| | 0_1-1_mismatch_A-C | | | | | | | | | |
| | 9_2-2_bulge-symmetric_UU-CC | | | | | | | | | |
| | 28_6-6_internal_loop- | | | | | | | | | |
| | symmetric_AGGCCA-GAUAGA | | | | | | | | | |
| | 40_4-4_bulge-symmetric_GAGU- | | | | | | | | | |
| | CAGA | | | | | | | | | |
| 2604 | −33_4-4_bulge-symmetric_UUCG- | GCCACACAGACCGGGUACGC | −18 | 32 | 0.03 | 0.831 | 0.764 | 0.93 | 1.672 | 1.538 |
| | ACAU | CUUUGAAAGUAUCAUGAAU | | | | | | | | |
| | −18_6-6_internal_loop- | ACAUCCACGGCUAACGAAU | | | | | | | | |
| | symmetric_UGGUGU-UGUUUU | UCCUUUUGUUUUCACUGUC | | | | | | | | |
| | −7_1-1_mismatch_A-C | GUACAUUGGCCACUCCCAG | | | | | | | | |
| | 0_1-1_mismatch_A-C | U | | | | | | | | |
| | 16_4-1_bulge-asymmetric_AAGG-A | | | | | | | | | |
| | 32_6-6_internal_loop- | | | | | | | | | |
| | symmetric_CAAGGA-GGGUAC | | | | | | | | | |
| | 40_4-4_bulge-symmetric_GAGU- | | | | | | | | | |
| | CAGA | | | | | | | | | |
| 2605 | −33_4-4_bulge-symmetric_UUCG- | GCCACACAGACCUCCUAACA | −8 | 28 | 0.415 | 0.957 | 0.956 | 1.211 | 1.729 | 1.716 |
| | ACAU | GGUUGAAAGUCCUUUCAUG | | | | | | | | |
| | −20_4-4_bulge-symmetric_UGGU- | AAUACAUCCACGGCUAAUU | | | | | | | | |
| | UAGU | UGCUACUUUACUAGUCACU | | | | | | | | |
| | −8_6-6_internal_loop- | GUCGUACAUUGGCCACUCCC | | | | | | | | |
| | symmetric_GAAUUC-UUGCUA | AGU | | | | | | | | |
| | 0_1-1_mismatch_A-C | | | | | | | | | |
| | 28_6-6_internal_loop- | | | | | | | | | |
| | symmetric_AGGCCA-AACAGG | | | | | | | | | |
| | 40_4-4_bulge-symmetric_GAGU- | | | | | | | | | |
| | CAGA | | | | | | | | | |
| 2606 | −33_4-4_bulge-symmetric_UUCG- | GCCACACAGACCUCCUAAAA | −18 | 28 | 0.046 | 0.931 | 0.909 | 0.934 | 1.701 | 1.638 |
| | ACAU | UGUUGAAAGUGCUUUCAGG | | | | | | | | |
| | −18_6-6_internal_loop- | AAUACAUCCACGGCUAAUG | | | | | | | | |
| | symmetric_UGGUGU-UUGUUU | AAUUCCUUUUUGUUUCACU | | | | | | | | |
| | 0_1-1_mismatch_A-C | GUCGUACAUUGGCCACUCCC | | | | | | | | |
| | 12_1-1_mismatch_A-G | AGU | | | | | | | | |
| | 19_1-1_mismatch_G-G | | | | | | | | | |
| | 28_6-6_internal_loop- | | | | | | | | | |
| | symmetric_AGGCCA-AAAAUG | | | | | | | | | |
| | 40_4-4_bulge-symmetric_GAGU- | | | | | | | | | |
| | CAGA | | | | | | | | | |
| 2607 | −33_4-4_bulge-symmetric_UUCG- | GCCACACAGACCAUUAGCGC | −6 | 32 | 0.147 | 0.919 | 0.917 | 0.89 | 1.006 | 1.042 |
| | ACAU | CUUUGAAAGUCCUUUGAUG | | | | | | | | |
| | −20_4-4_bulge-symmetric_UGGU- | CAUACAUCCACGGCUAUUU | | | | | | | | |
| | UAGU | AGGUCCUUUACUAGUCACU | | | | | | | | |
| | −6_6-6_internal_loop- | GUCGUACAUUGGCCACUCCC | | | | | | | | |
| | symmetric_AUUCAU-UUUAGG | AGU | | | | | | | | |
| | 0_1-1_mismatch_A-C | | | | | | | | | |
| | 10_1-1_mismatch_U-C | | | | | | | | | |
| | 14_1-1_mismatch_G-G | | | | | | | | | |
| | 32_6-6_internal_loop- | | | | | | | | | |
| | symmetric_CAAGGA-AUUAGC | | | | | | | | | |
| | 40_4-4_bulge-symmetric_GAGU- | | | | | | | | | |
| | CAGA | | | | | | | | | |
| 2608 | −33_4-4_bulge-symmetric_UUCG- | GCCACACAGACCUCCUGAUA | −8 | 28 | 0.09 | 0.849 | 0.865 | 0.978 | 1.324 | 1.382 |
| | ACAU | ACUUGAAAGUCCUUUGAUG | | | | | | | | |
| | −20_4-4_bulge-symmetric_UGGU- | CAUACAUCCACGGCUAAUC | | | | | | | | |
| | UAGU | UGGGGCUUUACUAGUCACU | | | | | | | | |
| | −8_6-6_internal_loop- | GUCGUACAUUGGCCACUCCC | | | | | | | | |
| | symmetric_GAAUUC-CUGGGG | AGU | | | | | | | | |
| | 0_1-1_mismatch_A-C | | | | | | | | | |
| | 10_1-1_mismatch_U-C | | | | | | | | | |
| | 14_1-1_mismatch_G-G | | | | | | | | | |
| | 28_6-6_internal_loop- | | | | | | | | | |
| | symmetric_AGGCCA-GAUAAC | | | | | | | | | |
| | 40_4-4_bulge-symmetric_GAGU- | | | | | | | | | |
| | CAGA | | | | | | | | | |
| 2609 | −33_4-4_bulge-symmetric_UUCG- | GCCACAUACAAGUCCUUGG | −8 | 38 | 0.238 | 0.937 | 0.933 | 0.614 | 0.737 | 0.791 |
| | ACAU | CCUUUGAAAGUCCUUUGAU | | | | | | | | |
| | −20_4-4_bulge-symmetric_UGGU- | GCAUACAUCCACGGCUAAU | | | | | | | | |
| | UAGU | UUAGUACUUUACUAGUCAC | | | | | | | | |
| | −8_6-6_internal_loop- | UGUCGUACAUUGGCCACUC | | | | | | | | |
| | symmetric_GAAUUC-UUAGUA | CCAGU | | | | | | | | |
| | 0_1-1_mismatch_A-C | | | | | | | | | |
| | 10_1-1_mismatch_U-C | | | | | | | | | |
| | 14_1-1_mismatch_G-G | | | | | | | | | |
| | 38_6-6_internal_loop- | | | | | | | | | |
| | symmetric_GGGAGU-UACAAG | | | | | | | | | |
| 2610 | −8_6-6_internal_loop- | GCCACAACUCCCUCGAACGA | −8 | 30 | 0.359 | 0.834 | 0.744 | 1.075 | 1.236 | 1.183 |
| | symmetric_GAAUUC-UAUAAG | CUUUGAAAGUCCUUUCAUG | | | | | | | | |
| | 0_1-1_mismatch_A-C | AAUAGAUCCACGGCUAAUU | | | | | | | | |
| | 6_1-1_mismatch_G-G | AUAAGCUUUACACCACACU | | | | | | | | |
| | 30_6-6_internal_loop- | GUCGUCGAAUGGCCACUCCC | | | | | | | | |
| | symmetric_GCCAAG-GAACGA | AGU | | | | | | | | |
| 2611 | −33_4-4_bulge-symmetric_UUCG- | GCCACAACUCCCUCGAACGA | −8 | 30 | 0.341 | 0.782 | 0.743 | 1.075 | 1.206 | 1.326 |
| | ACAU | CUUUGAAAGUCCUUUCAUG | | | | | | | | |
| | −20_4-4_bulge-symmetric_UGGU- | AAUAGAUCCACGGCUAAUU | | | | | | | | |
| | UAGU | AUAAGCUUUACUAGUCACU | | | | | | | | |
| | −8_6-6_internal_loop- | GUCGUACAUUGGCCACUCCC | | | | | | | | |
| | symmetric_GAAUUC-UAUAAG | AGU | | | | | | | | |
| | 0_1-1_mismatch_A-C | | | | | | | | | |
| | 6_1-1_mismatch_G-G | | | | | | | | | |
| | 30_6-6_internal_loop- | | | | | | | | | |
| | symmetric_GCCAAG-GAACGA | | | | | | | | | |
| 2612 | −6_6-6_internal_loop- | GCCACAACUCCCUCCUUGGC | −6 | 24 | 0.288 | 0.778 | 0.713 | 0.938 | 1.218 | 1.198 |
| | symmetric_AUUCAU-UGCCUG | AAGUCCAAGUCCUUUCAUG | | | | | | | | |
| | 0_1-1_mismatch_A-C | AAUAGAUCCACGGCUAUGC | | | | | | | | |
| | 6_1-1_mismatch_G-G | CUGUCCUUUACACCACACUG | | | | | | | | |
| | 24_6-6_internal_loop- | UCGUCGAAUGGCCACUCCCA | | | | | | | | |
| | symmetric_UCAAAG-AAGUCC | GU | | | | | | | | |
| 2613 | −33_4-4_bulge-symmetric_UUCG- | GCCACACAGACCUCCUUGGC | −6 | 24 | 0.27 | 0.917 | 0.911 | 1.136 | 1.65 | 1.595 |
| | ACAU | AAGUCCAAGUCCUUUCAUG | | | | | | | | |
| | −20_4-4_bulge-symmetric_UGGU- | AAUAGAUCCACGGCUAUGC | | | | | | | | |
| | UAGU | CUGUCCUUUACUAGUCACU | | | | | | | | |
| | −6_6-6_internal_loop- | GUCGUACAUUGGCCACUCCC | | | | | | | | |
| | symmetric_AUUCAU-UGCCUG | AGU | | | | | | | | |
| | 0_1-1_mismatch_A-C | | | | | | | | | |
| | 6_1-1_mismatch_G-G | | | | | | | | | |
| | 24_6-6_internal_loop- | | | | | | | | | |
| | symmetric_UCAAAG-AAGUCC | | | | | | | | | |
| | 40_4-4_bulge-symmetric_GAGU- | | | | | | | | | |
| | CAGA | | | | | | | | | |
| 2614 | −6_6-6_internal_loop- | GCCACACAGACCUCCUUGGC | −6 | 24 | 0.475 | 0.881 | 0.864 | 1.177 | 1.57 | 1.424 |
| | symmetric_AUUCAU-UGCCUG | AAGUCCAAGUCCUUUCAUG | | | | | | | | |
| | 0_1-1_mismatch_A-C | AAUAGAUCCACGGCUAUGC | | | | | | | | |
| | 6_1-1_mismatch_G-G | CUGUCCUUUACACCACACUG | | | | | | | | |
| | 24_6-6_internal_loop- | UCGUCGAAUGGCCACUCCCA | | | | | | | | |
| | symmetric_UCAAAG-AAGUCC | GU | | | | | | | | |
| | 40_4-4_bulge-symmetric_GAGU- | | | | | | | | | |
| | CAGA | | | | | | | | | |
| 2615 | −33_4-4_bulge-symmetric_UUCG- | GCCACAACUCCCUCCUUGGC | −6 | 24 | 0.302 | 0.783 | 0.806 | 0.955 | 1.235 | 1.28 |
| | ACAU | AAGUCCAAGUCCUUUCAUG | | | | | | | | |
| | −20_4-4_bulge-symmetric_UGGU- | AAUAGAUCCACGGCUAUGC | | | | | | | | |
| | UAGU | CUGUCCUUUACUAGUCACU | | | | | | | | |
| | −6_6-6_internal_loop- | GUCGUACAUUGGCCACUCCC | | | | | | | | |
| | symmetric_AUUCAU-UGCCUG | AGU | | | | | | | | |
| | 0_1-1_mismatch_A-C | | | | | | | | | |
| | 6_1-1_mismatch_G-G | | | | | | | | | |
| | 24_6-6_internal_loop- | | | | | | | | | |
| | symmetric_UCAAAG-AAGUCC | | | | | | | | | |
| 2616 | −12_6-6_internal_loop- | GCCACAACUCCCUCCUUGAA | −12 | 26 | 0.304 | 0.773 | 0.734 | 1.017 | 1.3 | 1.195 |
| | symmetric_AAAGGA-GGUUUG | AGCAGAAAGUCCUUUCGUG | | | | | | | | |
| | −4_3-2_bulge-asymmetric_UUA-AC | AAUACAUCCACGGCACUGA | | | | | | | | |
| | 0_1-1_mismatch_A-C | AUGGUUUGACACCACACUG | | | | | | | | |
| | 13_1-1_wobble_U-G | UCGUCGAAUGGCCACUCCCA | | | | | | | | |
| | 26_6-6_internal_loop- | GU | | | | | | | | |
| | symmetric_AAAGGC-AAAGCA | | | | | | | | | |
| 2617 | −12_6-6_internal_loop- | GCCACACAGACCUCCUUGAA | −12 | 26 | 0.639 | 0.922 | 0.873 | 1.391 | 1.641 | 1.649 |
| | symmetric_AAAGGA-GGUUUG | AGCAGAAAGUCCUUUCGUG | | | | | | | | |
| | −4_3-2_bulge-asymmetric_UUA-AC | AAUACAUCCACGGCACUGA | | | | | | | | |
| | 0_1-1_mismatch_A-C | AUGGUUUGACACCACACUG | | | | | | | | |
| | 13_1-1_wobble_U-G | UCGUCGAAUGGCCACUCCCA | | | | | | | | |
| | 26_6-6_internal_loop- | GU | | | | | | | | |
| | symmetric_AAAGGC-AAAGCA | | | | | | | | | |
| | 40_4-4_bulge-symmetric_GAGU- | | | | | | | | | |
| | CAGA | | | | | | | | | |
| 2618 | −18_6-6_internal_loop- | GCCACAACUCCCUCGCGCAA | −18 | 30 | 0.368 | 0.806 | 0.803 | 1.136 | 1.286 | 1.315 |
| | symmetric_UGGUGU-UGGAGU | CUUUGAAAGUCCUUUCAUG | | | | | | | | |
| | −6_3-3_bulge-symmetric_CAU-CAU | CAUAGAUCCACGGCUACAU | | | | | | | | |
| | 0_1-1_mismatch_A-C | AAUUCCUUUUGGAGUCACU | | | | | | | | |
| | 6_1-1_mismatch_G-G | GUCGUCGAAUGGCCACUCCC | | | | | | | | |
| | 10_1-1_mismatch_U-C | AGU | | | | | | | | |
| | 30_6-6_internal_loop- | | | | | | | | | |
| | symmetric_GCCAAG-GCGCAA | | | | | | | | | |
| 2619 | −18_6-6_internal_loop- | GCCACACAGACCUCGCGCAA | −18 | 30 | 0.459 | 0.895 | 0.872 | 1.247 | 1.496 | 1.552 |
| | symmetric_UGGUGU-UGGAGU | CUUUGAAAGUCCUUUCAUG | | | | | | | | |
| | −6_3-3_bulge-symmetric_CAU-CAU | CAUAGAUCCACGGCUACAU | | | | | | | | |
| | 0_1-1_mismatch_A-C | AAUUCCUUUUGGAGUCACU | | | | | | | | |
| | 6_1-1_mismatch_G-G | GUCGUCGAAUGGCCACUCCC | | | | | | | | |
| | 10_1-1_mismatch_U-C | AGU | | | | | | | | |
| | 30_6-6_internal_loop- | | | | | | | | | |
| | symmetric_GCCAAG-GCGCAA | | | | | | | | | |
| | 40_4-4_bulge-symmetric_GAGU- | | | | | | | | | |
| | CAGA | | | | | | | | | |
| 2620 | −33_4-4_bulge-symmetric_UUCG- | GCCACAACUCCCUCGCGCAA | −18 | 30 | 0.29 | 0.816 | 0.843 | 1.037 | 1.368 | 1.36 |
| | ACAU | CUUUGAAAGUCCUUUCAUG | | | | | | | | |
| | −18_6-6_internal_loop- | CAUAGAUCCACGGCUACAU | | | | | | | | |
| | symmetric_UGGUGU-UGGAGU | AAUUCCUUUUGGAGUCACU | | | | | | | | |
| | −6_3-3_bulge-symmetric_CAU-CAU | GUCGUACAUUGGCCACUCCC | | | | | | | | |
| | 0_1-1_mismatch_A-C | AGU | | | | | | | | |
| | 6_1-1_mismatch_G-G | | | | | | | | | |
| | 10_1-1_mismatch_U-C | | | | | | | | | |
| | 30_6-6_internal_loop- | | | | | | | | | |
| | symmetric_GCCAAG-GCGCAA | | | | | | | | | |
| 2621 | −10_6-6_internal_loop- | GCCACAACUCCCUCAGCCUA | −10 | 30 | 0.395 | 0.757 | 0.676 | 1.043 | 1.165 | 1.189 |
| | symmetric_AGGAAU-CAGUAC | CUUUGAAAGUCCUUUCAUC | | | | | | | | |
| | 0_1-1_mismatch_A-C | AAUACCACCACGGCUAAUG | | | | | | | | |
| | 4_2-2_bulge-symmetric_AU-CA | ACAGUACUUACACCACACU | | | | | | | | |
| | 11_1-1_mismatch_C-C | GUCGUCGAAUGGCCACUCCC | | | | | | | | |
| | 30_6-6_internal_loop- | AGU | | | | | | | | |
| | symmetric_GCCAAG-AGCCUA | | | | | | | | | |
| 2622 | −10_6-6_internal_loop- | GCCACACAGACCUCAGCCUA | −10 | 30 | 0.712 | 0.927 | 0.884 | 1.405 | 1.283 | 1.438 |
| | symmetric_AGGAAU-CAGUAC | CUUUGAAAGUCCUUUCAUC | | | | | | | | |
| | 0_1-1_mismatch_A-C | AAUACCACCACGGCUAAUG | | | | | | | | |
| | 4_2-2_bulge-symmetric_AU-CA | ACAGUACUUACACCACACU | | | | | | | | |
| | 11_1-1_mismatch_C-C | GUCGUCGAAUGGCCACUCCC | | | | | | | | |
| | 30_6-6_internal_loop- | AGU | | | | | | | | |
| | symmetric_GCCAAG-AGCCUA | | | | | | | | | |
| | 40_4-4_bulge-symmetric_GAGU- | | | | | | | | | |
| | CAGA | | | | | | | | | |
| 2623 | −33_4-4_bulge-symmetric_UUCG- | GCCACAACUCCCUCAGCCUA | −10 | 30 | 0.302 | 0.792 | 0.804 | 1.099 | 1.208 | 1.317 |
| | ACAU | CUUUGAAAGUCCUUUCAUC | | | | | | | | |
| | −20_4-4_bulge-symmetric_UGGU- | AAUACCACCACGGCUAAUG | | | | | | | | |
| | UAGU | ACAGUACUUACUAGUCACU | | | | | | | | |
| | −10_6-6_internal_loop- | GUCGUACAUUGGCCACUCCC | | | | | | | | |
| | symmetric_AGGAAU-CAGUAC | AGU | | | | | | | | |
| | 0_1-1_mismatch_A-C | | | | | | | | | |
| | 4_2-2_bulge-symmetric_AU-CA | | | | | | | | | |
| | 11_1-1_mismatch_C-C | | | | | | | | | |
| | 30_6-6_internal_loop- | | | | | | | | | |
| | symmetric_GCCAAG-AGCCUA | | | | | | | | | |
| 2624 | −14_6-6_internal_loop- | GCCACAACUCCCUCGGACAA | −14 | 30 | 0.465 | 0.751 | 0.744 | 1.127 | 1.166 | 1.25 |
| | symmetric_GUAAAG-GAACCG | CUUUGAAAGUCCUUUCAUG | | | | | | | | |
| | −5_4-4_bulge-symmetric_CAUU- | AAUACAACCACGGCUCAAC | | | | | | | | |
| | CAAC | AAUUCGAACCGACCACACU | | | | | | | | |
| | 0_1-1_mismatch_A-C | GUCGUCGAAUGGCCACUCCC | | | | | | | | |
| | 4_1-1_mismatch_A-A | AGU | | | | | | | | |
| | 30_6-6_internal_loop- | | | | | | | | | |
| | symmetric_GCCAAG-GGACAA | | | | | | | | | |
| 2625 | −14_6-6_internal_loop- | GCCACACAGACCUCGGACAA | −14 | 30 | 0.613 | 0.78 | 0.769 | 1.353 | 1.317 | 1.381 |
| | symmetric_GUAAAG-GAACCG | CUUUGAAAGUCCUUUCAUG | | | | | | | | |
| | −5_4-4_bulge-symmetric_CAUU- | AAUACAACCACGGCUCAAC | | | | | | | | |
| | CAAC | AAUUCGAACCGACCACACU | | | | | | | | |
| | 0_1-1_mismatch_A-C | GUCGUCGAAUGGCCACUCCC | | | | | | | | |
| | 4_1-1_mismatch_A-A | AGU | | | | | | | | |
| | 30_6-6_internal_loop- | | | | | | | | | |
| | symmetric_GCCAAG-GGACAA | | | | | | | | | |
| | 40_4-4_bulge-symmetric_GAGU- | | | | | | | | | |
| | CAGA | | | | | | | | | |
| 2626 | −14_6-6_internal_loop- | GCCACAACUCCCUCCUUGAU | −14 | 26 | 0.268 | 0.749 | 0.766 | 0.947 | 1.183 | 1.222 |
| | symmetric_GUAAAG-GAUCCA | GGCGGAAAGUCCUUUCAUG | | | | | | | | |
| | −7_1-1_mismatch_A-A | AAUACAUCCACGGCUAAAG | | | | | | | | |
| | 0_1-1_mismatch_A-C | AAUUCGAUCCAACCACACU | | | | | | | | |
| | 26_6-6_internal_loop- | GUCGUCGAAUGGCCACUCCC | | | | | | | | |
| | symmetric_AAAGGC-AUGGCG | AGU | | | | | | | | |
| 2627 | −14_6-6_internal_loop- | GCCACACAGACCUCCUUGAU | −14 | 26 | 0.398 | 0.895 | 0.871 | 1.122 | 1.534 | 1.463 |
| | symmetric_GUAAAG-GAUCCA | GGCGGAAAGUCCUUUCAUG | | | | | | | | |
| | −7_1-1_mismatch_A-A | AAUACAUCCACGGCUAAAG | | | | | | | | |
| | 0_1-1_mismatch_A-C | AAUUCGAUCCAACCACACU | | | | | | | | |
| | 26_6-6_internal_loop- | GUCGUCGAAUGGCCACUCCC | | | | | | | | |
| | symmetric_AAAGGC-AUGGCG | AGU | | | | | | | | |
| | 40_4-4_bulge-symmetric_GAGU- | | | | | | | | | |
| | CAGA | | | | | | | | | |
| 2628 | −6_6-6_internal_loop- | GCCACAACUCCCUCGACUAA | −6 | 30 | 0.331 | 0.817 | 0.824 | 1.013 | 1.334 | 1.325 |
| | symmetric_AUUCAU-CUAGUG | CUUUGAAAGUCCUUUCAUG | | | | | | | | |
| | 0_1-1_mismatch_A-C | AAUAGCUCCACGGCUACUA | | | | | | | | |
| | 5_2-2_bulge-symmetric_UG-GC | GUGUCCUUUACACCACACU | | | | | | | | |
| | 30_6-6_internal_loop- | GUCGUCGAAUGGCCACUCCC | | | | | | | | |
| | symmetric_GCCAAG-GACUAA | AGU | | | | | | | | |
| 2629 | −6_6-6_internal_loop- | GCCACACAGACCUCGACUAA | −6 | 30 | 0.633 | 0.919 | 0.917 | 1.365 | 1.65 | 1.631 |
| | symmetric_AUUCAU-CUAGUG | CUUUGAAAGUCCUUUCAUG | | | | | | | | |
| | 0_1-1_mismatch_A-C | AAUAGCUCCACGGCUACUA | | | | | | | | |
| | 5_2-2_bulge-symmetric_UG-GC | GUGUCCUUUACACCACACU | | | | | | | | |
| | 30_6-6_internal_loop- | GUCGUCGAAUGGCCACUCCC | | | | | | | | |
| | symmetric_GCCAAG-GACUAA | AGU | | | | | | | | |
| | 40_4-4_bulge-symmetric_GAGU- | | | | | | | | | |
| | CAGA | | | | | | | | | |
| 2630 | −33_4-4_bulge-symmetric_UUCG- | GCCACAACUCCCUCGACUAA | −6 | 30 | 0.276 | 0.798 | 0.808 | 1.043 | 1.31 | 1.299 |
| | ACAU | CUUUGAAAGUCCUUUCAUG | | | | | | | | |
| | −20_4-4_bulge-symmetric_UGGU- | AAUAGCUCCACGGCUACUA | | | | | | | | |
| | UAGU | GUGUCCUUUACUAGUCACU | | | | | | | | |
| | −6_6-6_internal_loop- | GUCGUACAUUGGCCACUCCC | | | | | | | | |
| | symmetric_AUUCAU-CUAGUG | AGU | | | | | | | | |
| | 0_1-1_mismatch_A-C | | | | | | | | | |
| | 5_2-2_bulge-symmetric_UG-GC | | | | | | | | | |
| | 30_6-6_internal_loop- | | | | | | | | | |
| | symmetric_GCCAAG-GACUAA | | | | | | | | | |
| 2631 | −8_6-6_internal_loop- | GCCACAACUCCCUCCUAUAG | −8 | 28 | 0.381 | 0.759 | 0.749 | 1.048 | 1.145 | 1.165 |
| | symmetric_GAAUUC-CGCCGA | AGUUGAAAGUCCUUUCAUG | | | | | | | | |
| | 0_1-1_mismatch_A-C | AAUACAUCUACGGCUAAUC | | | | | | | | |
| | 2_1-1_wobble_G-U | GCCGACUUUACACCACACUG | | | | | | | | |
| | 28_6-6_internal_loop- | UCGUCGAAUGGCCACUCCCA | | | | | | | | |
| | symmetric_AGGCCA-AUAGAG | GU | | | | | | | | |
| 2632 | −16_6-6_internal_loop- | GCCACAACUCCCUCCUCUCA | −16 | 28 | 0.399 | 0.812 | 0.81 | 0.999 | 1.185 | 1.338 |
| | symmetric_GUGUAA-AAUGAA | AGUUGAAAGUCCUUUCAUG | | | | | | | | |
| | −5_4-1_bulge-asymmetric_CAUU-U | AAUAUAUCCACGGCUUAAU | | | | | | | | |
| | 0_1-1_mismatch_A-C | UCCUAAUGAACACACUGUC | | | | | | | | |
| | 6_1-1_wobble_G-U | GUCGAAUGGCCACUCCCAG | | | | | | | | |
| | 28_6-6_internal_loop- | U | | | | | | | | |
| | symmetric_AGGCCA-CUCAAG | | | | | | | | | |
| 2633 | −6_6-6_internal_loop- | GCCACAACUCCCUCGCAUCA | −6 | 30 | 0.243 | 0.822 | 0.771 | 0.97 | 1.234 | 1.231 |
| | symmetric_AUUCAU-CGUCGC | CUUUGAAAGUACUUUCAUU | | | | | | | | |
| | 0_1-1_mismatch_A-C | AAUACAUCCACGGCUACGU | | | | | | | | |
| | 11_1-1_mismatch_C-U | CGCUCCUUUACACCACACUG | | | | | | | | |
| | 19_1-1_mismatch_G-A | UCGUCGAAUGGCCACUCCCA | | | | | | | | |
| | 30_6-6_internal_loop- | GU | | | | | | | | |
| | symmetric_GCCAAG-GCAUCA | | | | | | | | | |
| 2634 | −16_6-6_internal_loop- | GCCACAACUCCCUCAAAACA | −16 | 30 | 0.24 | 0.834 | 0.682 | 0.946 | 1.215 | 1.366 |
| | symmetric_GUGUAA-GAGAUG | CUUUGAAAGUCCUUUCAUG | | | | | | | | |
| | −4_1-0_bulge-asymmetric_A- | AAUGUCUCCACGGCAAUGA | | | | | | | | |
| | 0_1-1_mismatch_A-C | AUUCCUGAGAUGCACACUG | | | | | | | | |
| | 5_2-2_bulge-symmetric_UG-UC | UCGUCGAAUGGCCACUCCCA | | | | | | | | |
| | 7_1-1_wobble_U-G | GU | | | | | | | | |
| | 30_6-6_internal_loop- | | | | | | | | | |
| | symmetric_GCCAAG-AAAACA | | | | | | | | | |
| 2635 | −12_6-6_internal_loop- | GCCACAACUCCCUCCUUGAU | −12 | 26 | 0.272 | 0.774 | 0.747 | 0.961 | 1.164 | 1.281 |
| | symmetric_AAAGGA-AGUAAA | AAUCGAAAGUCCUUUCAUG | | | | | | | | |
| | −2_2-1_bulge-asymmetric_GC-A | AAUACAUCCACGAUAAUGA | | | | | | | | |
| | 0_1-1_mismatch_A-C | AUAGUAAAACACCACACUG | | | | | | | | |
| | 26_6-6_internal_loop- | UCGUCGAAUGGCCACUCCCA | | | | | | | | |
| | symmetric_AAAGGC-AUAAUC | GU | | | | | | | | |
| 2636 | −8_6-6_internal_loop- | GCCACAACUCCCUCCUUGAU | −8 | 26 | 0.254 | 0.784 | 0.77 | 0.898 | 1.212 | 1.225 |
| | symmetric_GAAUUC-AAAACA | AGUGGAAAGUCCUUUCAUG | | | | | | | | |
| | −6_1-1_wobble_U-G | AUUACAUCCACGGCUAGUA | | | | | | | | |
| | 0_1-1_mismatch_A-C | AAACACUUUACACCACACU | | | | | | | | |
| | 9_1-1_mismatch_U-U | GUCGUCGAAUGGCCACUCCC | | | | | | | | |
| | 26_6-6_internal_loop- | AGU | | | | | | | | |
| | symmetric_AAAGGC-AUAGUG | | | | | | | | | |
| 2637 | −33_4-4_bulge-symmetric_UUCG- | GCCACACAGACCUCCUUGAU | −8 | 26 | 0.256 | 0.901 | 0.902 | 1.092 | 1.609 | 1.536 |
| | ACAU | AGUGGAAAGUCCUUUCAUG | | | | | | | | |
| | −20_4-4_bulge-symmetric_UGGU- | AUUACAUCCACGGCUAGUA | | | | | | | | |
| | UAGU | AAACACUUUACUAGUCACU | | | | | | | | |
| | −8_6-6_internal_loop- | GUCGUACAUUGGCCACUCCC | | | | | | | | |
| | symmetric_GAAUUC-AAAACA | AGU | | | | | | | | |
| | −6_1-1_wobble_U-G | | | | | | | | | |
| | 0_1-1_mismatch_A-C | | | | | | | | | |
| | 9_1-1_mismatch_U-U | | | | | | | | | |
| | 26_6-6_internal_loop- | | | | | | | | | |
| | symmetric_AAAGGC-AUAGUG | | | | | | | | | |
| | 40_4-4_bulge-symmetric_GAGU- | | | | | | | | | |
| | CAGA | | | | | | | | | |
| 2638 | −10_6-6_internal_loop- | GCCACAACUCCCUCCUAGUA | −10 | 28 | 0.31 | 0.741 | 0.734 | 0.99 | 1.234 | 1.218 |
| | symmetric_AGGAAU-UCUGAG | GGUUGAAAGUCCUUUCAUU | | | | | | | | |
| | 0_1-1_mismatch_A-C | AAUAACUCCACGGCUAAUG | | | | | | | | |
| | 5_2-2_bulge-symmetric_UG-AC | AUCUGAGUUACACCACACU | | | | | | | | |
| | 11_1-1_mismatch_C-U | GUCGUCGAAUGGCCACUCCC | | | | | | | | |
| | 28_6-6_internal_loop- | AGU | | | | | | | | |
| | symmetric_AGGCCA-AGUAGG | | | | | | | | | |
| 2639 | −18_6-6_internal_loop- | GCCACAACUCCCUCCUUGGC | −18 | 24 | 0.322 | 0.814 | 0.769 | 0.937 | 1.235 | 1.283 |
| | symmetric_UGGUGU-UGAAGU | AACGCUAAGUCCUUUCAUG | | | | | | | | |
| | −5_1-1_mismatch_U-C | AAUACAUCCACGGCUCAUG | | | | | | | | |
| | 0_1-1_mismatch_A-C | AAUUCCUUUUGAAGUCACU | | | | | | | | |
| | 24_6-6_internal_loop- | GUCGUCGAAUGGCCACUCCC | | | | | | | | |
| | symmetric_UCAAAG-AACGCU | AGU | | | | | | | | |
| 2640 | −33_4-4_bulge-symmetric_UUCG- | GCCACACAGACCUCCUUGGC | −18 | 24 | 0.283 | 0.914 | 0.911 | 1.064 | 1.58 | 1.513 |
| | ACAU | AACGCUAAGUCCUUUCAUG | | | | | | | | |
| | −18_6-6_internal_loop- | AAUACAUCCACGGCUCAUG | | | | | | | | |
| | symmetric_UGGUGU-UGAAGU | AAUUCCUUUUGAAGUCACU | | | | | | | | |
| | −5_1-1_mismatch_U-C | GUCGUACAUUGGCCACUCCC | | | | | | | | |
| | 0_1-1_mismatch_A-C | AGU | | | | | | | | |
| | 24_6-6_internal_loop- | | | | | | | | | |
| | symmetric_UCAAAG-AACGCU | | | | | | | | | |
| | 40_4-4_bulge-symmetric_GAGU- | | | | | | | | | |
| | CAGA | | | | | | | | | |
| 2641 | −14_6-6_internal_loop- | GCCACAACUCCCUCCUUGGC | −14 | 24 | 0.284 | 0.749 | 0.728 | 0.916 | 1.26 | 1.231 |
| | symmetric_GUAAAG-GACCUG | AACGCUAAGUCCUUUCAUG | | | | | | | | |
| | −5_1-1_mismatch_U-C | AAUACAUCCACGGCUCAUG | | | | | | | | |
| | 0_1-1_mismatch_A-C | AAUUCGACCUGACCACACU | | | | | | | | |
| | 24_6-6_internal_loop- | GUCGUCGAAUGGCCACUCCC | | | | | | | | |
| | symmetric_UCAAAG-AACGCU | AGU | | | | | | | | |
| 2642 | −16_6-6_internal_loop- | GCCACAACUCCCUCCUACAU | −16 | 28 | 0.247 | 0.741 | 0.65 | 1.002 | 1.208 | 1.101 |
| | symmetric_GUGUAA-AUUUUA | UAUUGAAAGUCCUUUCAUG | | | | | | | | |
| | −4_2-0_bulge-asymmetric_UA- | AAUCCAUCCACGGCAUGAA | | | | | | | | |
| | 0_1-1_mismatch_A-C | UUCCUAUUUUACACACUGU | | | | | | | | |
| | 7_1-1_mismatch_U-C | CGUCGAAUGGCCACUCCCAG | | | | | | | | |
| | 28_6-6_internal_loop- | U | | | | | | | | |
| | symmetric_AGGCCA-ACAUUA | | | | | | | | | |
| 2643 | −6_6-6_internal_loop- | GCCACAACUCCCGAAGCCGC | −6 | 32 | 0.367 | 0.799 | 0.785 | 1.032 | 1.225 | 1.122 |
| | symmetric_AUUCAU-CCGCCC | CUUUGAAAGUCCUUUCAUG | | | | | | | | |
| | 0_1-1_mismatch_A-C | AAUAGAUCCACGGCUACCG | | | | | | | | |
| | 6_1-1_mismatch_G-G | CCCUCCUUUACACCACACUG | | | | | | | | |
| | 32_6-6_internal_loop- | UCGUCGAAUGGCCACUCCCA | | | | | | | | |
| | symmetric_CAAGGA-GAAGCC | GU | | | | | | | | |
| 2644 | −33_4-4_bulge-symmetric_UUCG- | GCCACACAGACCGAAGCCGC | −6 | 32 | 0.408 | 0.921 | 0.915 | 1.18 | 1.442 | 1.532 |
| | ACAU | CUUUGAAAGUCCUUUCAUG | | | | | | | | |
| | −20_4-4_bulge-symmetric_UGGU- | AAUAGAUCCACGGCUACCG | | | | | | | | |
| | UAGU | CCCUCCUUUACUAGUCACUG | | | | | | | | |
| | −6_6-6_internal_loop- | UCGUACAUUGGCCACUCCCA | | | | | | | | |
| | symmetric_AUUCAU-CCGCCC | GU | | | | | | | | |
| | 0_1-1_mismatch_A-C | | | | | | | | | |
| | 6_1-1_mismatch_G-G | | | | | | | | | |
| | 32_6-6_internal_loop- | | | | | | | | | |
| | symmetric_CAAGGA-GAAGCC | | | | | | | | | |
| | 40_4-4_bulge-symmetric_GAGU- | | | | | | | | | |
| | CAGA | | | | | | | | | |
| 2645 | −14_6-6_internal_loop- | GCCACAACUCCCUCGACACG | −14 | 30 | 0.315 | 0.814 | 0.764 | 1.035 | 1.244 | 1.315 |
| | symmetric_GUAAAG-GGAAUG | CUUUGAAAGUCCUUUCAUG | | | | | | | | |
| | −5_2-0_bulge-asymmetric_UU- | AAUACAUCCACGGCUUGAA | | | | | | | | |
| | 0_1-1_mismatch_A-C | UUCGGAAUGACCACACUGU | | | | | | | | |
| | 30_6-6_internal_loop- | CGUCGAAUGGCCACUCCCAG | | | | | | | | |
| | symmetric_GCCAAG-GACACG | U | | | | | | | | |
| 2646 | −8_6-6_internal_loop- | GCCACAACUCCCUCCUUGGC | −8 | 24 | 0.273 | 0.806 | 0.677 | 0.895 | 1.18 | 1.198 |
| | symmetric_GAAUUC-CCUUGG | GGGGUUAAGUCCUUUCAUG | | | | | | | | |
| | 0_1-1_mismatch_A-C | AAUACACCCACGGCUAAUCC | | | | | | | | |
| | 4_1-1_mismatch_A-C | UUGGCUUUACACCACACUG | | | | | | | | |
| | 24_6-6_internal_loop- | UCGUCGAAUGGCCACUCCCA | | | | | | | | |
| | symmetric_UCAAAG-GGGGUU | GU | | | | | | | | |
| 2647 | −16_6-6_internal_loop- | GCCACAACUCCCUCCUUGUA | −16 | 26 | 0.264 | 0.792 | 0.739 | 0.943 | 1.224 | 1.3 |
| | symmetric_GUGUAA-CCUCUA | GGAGGAAAGUCCUUUCAUU | | | | | | | | |
| | −7_0-1_bulge-asymmetric_-G | AAUACAUCCACGGCUAAUG | | | | | | | | |
| | 0_1-1_mismatch_A-C | GAAUUCCUCCUCUACACACU | | | | | | | | |
| | 11_1-1_mismatch_C-U | GUCGUCGAAUGGCCACUCCC | | | | | | | | |
| | 26_6-6_internal_loop- | AGU | | | | | | | | |
| | symmetric_AAAGGC-UAGGAG | | | | | | | | | |
| 2648 | −10_6-6_internal_loop- | GCCACAACUCCCUCCUUGUA | −10 | 26 | 0.34 | 0.777 | 0.737 | 0.926 | 1.183 | 1.175 |
| | symmetric_AGGAAU-CGAAGA | ACGCGAAAGUCCUUUCAUG | | | | | | | | |
| | −5_1-1_mismatch_U-C | AAUACAUCCACGGCUCAUG | | | | | | | | |
| | 0_1-1_mismatch_A-C | ACGAAGAUUACACCACACU | | | | | | | | |
| | 26_6-6_internal_loop- | GUCGUCGAAUGGCCACUCCC | | | | | | | | |
| | symmetric_AAAGGC-UAACGC | AGU | | | | | | | | |
| 2649 | −33_4-4_bulge-symmetric_UUCG- | GCCACACAGACCUCCUUGUA | −10 | 26 | 0.306 | 0.886 | 0.887 | 1.097 | 1.499 | 1.415 |
| | ACAU | ACGCGAAAGUCCUUUCAUG | | | | | | | | |
| | −20_4-4_bulge-symmetric_UGGU- | AAUACAUCCACGGCUCAUG | | | | | | | | |
| | UAGU | ACGAAGAUUACUAGUCACU | | | | | | | | |
| | −10_6-6_internal_loop- | GUCGUACAUUGGCCACUCCC | | | | | | | | |
| | symmetric_AGGAAU-CGAAGA | AGU | | | | | | | | |
| | −5_1-1_mismatch_U-C | | | | | | | | | |
| | 0_1-1_mismatch_A-C | | | | | | | | | |
| | 26_6-6_internal_loop- | | | | | | | | | |
| | symmetric_AAAGGC-UAACGC | | | | | | | | | |
| | 40_4-4_bulge-symmetric_GAGU- | | | | | | | | | |
| | CAGA | | | | | | | | | |
| 2650 | −10_6-6_internal_loop- | GCCACAACUCCCUCCUAUCA | −10 | 28 | 0.351 | 0.803 | 0.809 | 1.029 | 1.227 | 1.302 |
| | symmetric_AGGAAU-CGCAGA | ACUUGAAAGUCCUUUCAUG | | | | | | | | |
| | 0_1-1_mismatch_A-C | CAUACAUCCACGGCUAAUG | | | | | | | | |
| | 10_1-1_mismatch_U-C | ACGCAGAUUACACCACACU | | | | | | | | |
| | 28_6-6_internal_loop- | GUCGUCGAAUGGCCACUCCC | | | | | | | | |
| | symmetric_AGGCCA-AUCAAC | AGU | | | | | | | | |
| 2651 | −33_4-4_bulge-symmetric_UUCG- | GCCACACAGACCUCCUAUCA | −10 | 28 | 0.259 | 0.886 | 0.886 | 1.121 | 1.502 | 1.536 |
| | ACAU | ACUUGAAAGUCCUUUCAUG | | | | | | | | |
| | −20_4-4_bulge-symmetric_UGGU- | CAUACAUCCACGGCUAAUG | | | | | | | | |
| | UAGU | ACGCAGAUUACUAGUCACU | | | | | | | | |
| | −10_6-6_internal_loop- | GUCGUACAUUGGCCACUCCC | | | | | | | | |
| | symmetric_AGGAAU-CGCAGA | AGU | | | | | | | | |
| | 0_1-1_mismatch_A-C | | | | | | | | | |
| | 10_1-1_mismatch_U-C | | | | | | | | | |
| | 28_6-6_internal_loop- | | | | | | | | | |
| | symmetric_AGGCCA-AUCAAC | | | | | | | | | |
| | 40_4-4_bulge-symmetric_GAGU- | | | | | | | | | |
| | CAGA | | | | | | | | | |
| 2652 | −14_6-6_internal_loop- | GCCACAACUCCCUCAGCCAG | −14 | 30 | 0.246 | 0.83 | 0.823 | 1.062 | 1.396 | 1.303 |
| | symmetric_GUAAAG-GACGCA | CUUUGAAAGUACUUUCAUG | | | | | | | | |
| | −6_1-1_wobble_U-G | AAUACAUCCACGGAGUGAA | | | | | | | | |
| | −3_2-0_bulge-asymmetric_AG- | UUCGACGCAACCACACUGUC | | | | | | | | |
| | 0_1-1_mismatch_A-C | GUCGAAUGGCCACUCCCAG | | | | | | | | |
| | 19_1-1_mismatch_G-A | U | | | | | | | | |
| | 30_6-6_internal_loop- | | | | | | | | | |
| | symmetric_GCCAAG-AGCCAG | | | | | | | | | |
| 2653 | −6_6-6_internal_loop- | GCCACAACUCCCAGGAUCGC | −6 | 32 | 0.195 | 0.889 | 0.839 | 0.921 | 1.436 | 1.309 |
| | symmetric_AUUCAU-CGACAG | CUUUGAAAGUCCUUCCCUG | | | | | | | | |
| | 0_1-1_mismatch_A-C | AAUACAUCCACGGCUACGA | | | | | | | | |
| | 13_1-1_mismatch_U-C | CAGUCCUUUACACCACACUG | | | | | | | | |
| | 15_1-1_mismatch_A-C | UCGUCGAAUGGCCACUCCCA | | | | | | | | |
| | 32_6-6_internal_loop- | GU | | | | | | | | |
| | symmetric_CAAGGA-AGGAUC | | | | | | | | | |
| 2654 | −10_6-6_internal_loop- | GCCACAACUCCCUCCUCUCG | −10 | 28 | 0.303 | 0.798 | 0.781 | 1.006 | 1.222 | 1.183 |
| | symmetric_AGGAAU-UAGAGC | AGUUGAAAGUCCUUUCAUG | | | | | | | | |
| | −6_2-2_bulge-symmetric_AU-CG | AAUACAUCCACGGCUACGG | | | | | | | | |
| | 0_1-1_mismatch_A-C | AUAGAGCUUACACCACACU | | | | | | | | |
| | 28_6-6_internal_loop- | GUCGUCGAAUGGCCACUCCC | | | | | | | | |
| | symmetric_AGGCCA-CUCGAG | AGU | | | | | | | | |
| 2655 | −33_4-4_bulge-symmetric_UUCG- | GCCACACAGACCUCCUCUCG | −10 | 28 | 0.246 | 0.838 | 0.831 | 1.047 | 1.493 | 1.473 |
| | ACAU | AGUUGAAAGUCCUUUCAUG | | | | | | | | |
| | −20_4-4_bulge-symmetric_UGGU- | AAUACAUCCACGGCUACGG | | | | | | | | |
| | UAGU | AUAGAGCUUACUAGUCACU | | | | | | | | |
| | −10_6-6_internal_loop- | GUCGUACAUUGGCCACUCCC | | | | | | | | |
| | symmetric_AGGAAU-UAGAGC | AGU | | | | | | | | |
| | −6_2-2_bulge-symmetric_AU-CG | | | | | | | | | |
| | 0_1-1_mismatch_A-C | | | | | | | | | |
| | 28_6-6_internal_loop- | | | | | | | | | |
| | symmetric_AGGCCA-CUCGAG | | | | | | | | | |
| | 40_4-4_bulge-symmetric_GAGU- | | | | | | | | | |
| | CAGA | | | | | | | | | |
| 2656 | −18_6-6_internal_loop- | GCCACAACUCCCUCCUCUCA | −18 | 28 | 0.115 | 0.819 | 0.831 | 0.936 | 1.259 | 1.278 |
| | symmetric_UGGUGU-UGUAAU | UCUUGAAAGUCGACUUUCA | | | | | | | | |
| | −3_2-0_bulge-asymmetric_AG- | UGAAUACAUCCACGGAAUG | | | | | | | | |
| | 0_1-1_mismatch_A-C | AAUUCCUUUUGUAAUCACU | | | | | | | | |
| | 18_0-2_bulge-asymmetric_-GA | GUCGUCGAAUGGCCACUCCC | | | | | | | | |
| | 28_6-6_internal_loop- | AGU | | | | | | | | |
| | symmetric_AGGCCA-CUCAUC | | | | | | | | | |
| 2657 | −6_6-6_internal_loop- | GCCACAACUCCCUCCUUGAU | −6 | 26 | 0.285 | 0.748 | 0.751 | 0.964 | 1.192 | 1.153 |
| | symmetric_AUUCAU-UCAUAC | AGGCGAAAGUCCUUUCAUG | | | | | | | | |
| | 0_1-1_mismatch_A-C | AAUAACUCCACGGCUAUCA | | | | | | | | |
| | 5_2-2_bulge-symmetric_UG-AC | UACUCCUUUACACCACACUG | | | | | | | | |
| | 26_6-6_internal_loop- | UCGUCGAAUGGCCACUCCCA | | | | | | | | |
| | symmetric_AAAGGC-AUAGGC | GU | | | | | | | | |
| 2658 | −8_6-6_internal_loop- | GCCACAACUCCCUCCUCUAA | −8 | 28 | 0.396 | 0.808 | 0.826 | 1.089 | 1.234 | 1.366 |
| | symmetric_GAAUUC-AACUGG | AGUUGAAAGUCCUUUCAUG | | | | | | | | |
| | −2_2-1_bulge-asymmetric_GC-A | AAUACAUCCACGAUAAUAA | | | | | | | | |
| | 0_1-1_mismatch_A-C | CUGGCUUUACACCACACUG | | | | | | | | |
| | 28_6-6_internal_loop- | UCGUCGAAUGGCCACUCCCA | | | | | | | | |
| | symmetric_AGGCCA-CUAAAG | GU | | | | | | | | |
| 2659 | −33_4-4_bulge-symmetric_UUCG- | GCCACACAGACCUCCUCUAA | −8 | 28 | 0.349 | 0.909 | 0.883 | 1.166 | 1.632 | 1.591 |
| | ACAU | AGUUGAAAGUCCUUUCAUG | | | | | | | | |
| | −20_4-4_bulge-symmetric_UGGU- | AAUACAUCCACGAUAAUAA | | | | | | | | |
| | UAGU | CUGGCUUUACUAGUCACUG | | | | | | | | |
| | −8_6-6_internal_loop- | UCGUACAUUGGCCACUCCCA | | | | | | | | |
| | symmetric_GAAUUC-AACUGG | GU | | | | | | | | |
| | −2_2-1_bulge-asymmetric_GC-A | | | | | | | | | |
| | 0_1-1_mismatch_A-C | | | | | | | | | |
| | 28_6-6_internal_loop- | | | | | | | | | |
| | symmetric_AGGCCA-CUAAAG | | | | | | | | | |
| | 40_4-4_bulge-symmetric_GAGU- | | | | | | | | | |
| | CAGA | | | | | | | | | |
| 2660 | −10_6-6_internal_loop- | GCCACAACUCCCUCCUGAUA | −10 | 28 | 0.315 | 0.808 | 0.776 | 1.011 | 1.252 | 1.152 |
| | symmetric_AGGAAU-CAAGAA | UGUUGAAAGUCCUUUCCUG | | | | | | | | |
| | −6_2-1_bulge-asymmetric_AU-C | AAUACAUCCACGGCUACGA | | | | | | | | |
| | 0_1-1_mismatch_A-C | CAAGAAUUACACCACACUG | | | | | | | | |
| | 13_1-1_mismatch_U-C | UCGUCGAAUGGCCACUCCCA | | | | | | | | |
| | 28_6-6_internal_loop- | GU | | | | | | | | |
| | symmetric_AGGCCA-GAUAUG | | | | | | | | | |
| 2661 | −33_4-4_bulge-symmetric_UUCG- | GCCACACAGACCUCCUGAUA | −10 | 28 | 0.205 | 0.897 | 0.893 | 1.062 | 1.621 | 1.619 |
| | ACAU | UGUUGAAAGUCCUUUCCUG | | | | | | | | |
| | −20_4-4_bulge-symmetric_UGGU- | AAUACAUCCACGGCUACGA | | | | | | | | |
| | UAGU | CAAGAAUUACUAGUCACUG | | | | | | | | |
| | −10_6-6_internal_loop- | UCGUACAUUGGCCACUCCCA | | | | | | | | |
| | symmetric_AGGAAU-CAAGAA | GU | | | | | | | | |
| | −6_2-1_bulge-asymmetric_AU-C | | | | | | | | | |
| | 0_1-1_mismatch_A-C | | | | | | | | | |
| | 13_1-1_mismatch_U-C | | | | | | | | | |
| | 28_6-6_internal_loop- | | | | | | | | | |
| | symmetric_AGGCCA-GAUAUG | | | | | | | | | |
| | 40_4-4_bulge-symmetric_GAGU- | | | | | | | | | |
| | CAGA | | | | | | | | | |
| 2662 | −10_6-6_internal_loop- | GCCACAACUCCCUCCUACAA | −10 | 28 | 0.329 | 0.785 | 0.82 | 1.02 | 1.183 | 1.248 |
| | symmetric_AGGAAU-CGGAGA | ACUUGAAAGUCCUUUCAUG | | | | | | | | |
| | −6_0-1_bulge-asymmetric_-U | AAUACAACCACGGCUAAUU | | | | | | | | |
| | 0_1-1_mismatch_A-C | GACGGAGAUUACACCACAC | | | | | | | | |
| | 4_1-1_mismatch_A-A | UGUCGUCGAAUGGCCACUC | | | | | | | | |
| | 28_6-6_internal_loop- | CCAGU | | | | | | | | |
| | symmetric_AGGCCA-ACAAAC | | | | | | | | | |
| 2663 | −33_4-4_bulge-symmetric_UUCG- | GCCACACAGACCUCCUACAA | −10 | 28 | 0.371 | 0.875 | 0.894 | 1.193 | 1.48 | 1.471 |
| | ACAU | ACUUGAAAGUCCUUUCAUG | | | | | | | | |
| | −20_4-4_bulge-symmetric_UGGU- | AAUACAACCACGGCUAAUU | | | | | | | | |
| | UAGU | GACGGAGAUUACUAGUCAC | | | | | | | | |
| | −10_6-6_internal_loop- | UGUCGUACAUUGGCCACUC | | | | | | | | |
| | symmetric_AGGAAU-CGGAGA | CCAGU | | | | | | | | |
| | −6_0-1_bulge-asymmetric_-U | | | | | | | | | |
| | 0_1-1_mismatch_A-C | | | | | | | | | |
| | 4_1-1_mismatch_A-A | | | | | | | | | |
| | 28_6-6_internal_loop- | | | | | | | | | |
| | symmetric_AGGCCA-ACAAAC | | | | | | | | | |
| | 40_4-4_bulge-symmetric_GAGU- | | | | | | | | | |
| | CAGA | | | | | | | | | |
| 2664 | −10_6-6_internal_loop- | GCCACAACUCCCUCCUGAAU | −10 | 28 | 0.333 | 0.794 | 0.771 | 1.081 | 1.228 | 1.21 |
| | symmetric_AGGAAU-UCUGAG | GAUUGAAAGUCCUUUCAUG | | | | | | | | |
| | −4_2-0_bulge-asymmetric_UA- | AAUACAUCCACGGCAUGAU | | | | | | | | |
| | 0_1-1_mismatch_A-C | CUGAGUUACACCACACUGU | | | | | | | | |
| | 28_6-6_internal_loop- | CGUCGAAUGGCCACUCCCAG | | | | | | | | |
| | symmetric_AGGCCA-GAAUGA | U | | | | | | | | |
| 2665 | −16_6-6_internal_loop- | GCCACAACUCCCUCCUCAUG | −16 | 28 | 0.215 | 0.767 | 0.77 | 0.949 | 1.164 | 1.242 |
| | symmetric_GUGUAA-GGAAUG | AGUUGAAAGUCCUUUCAUG | | | | | | | | |
| | −3_4-3_bulge-asymmetric_UUAG- | AAUACAUCCACGGACUUGA | | | | | | | | |
| | ACU | AUUCCUGGAAUGCACACUG | | | | | | | | |
| | 0_1-1_mismatch_A-C | UCGUCGAAUGGCCACUCCCA | | | | | | | | |
| | 28_6-6_internal_loop- | GU | | | | | | | | |
| | symmetric_AGGCCA-CAUGAG | | | | | | | | | |
| 2666 | −14_6-6_internal_loop- | GCCACAACUCCCUCCUUGUA | −14 | 26 | 0.29 | 0.757 | 0.781 | 0.879 | 1.187 | 1.213 |
| | symmetric_GUAAAG-GCGCUG | AGGAGAAAGUCCUUUCAUG | | | | | | | | |
| | −4_3-3_bulge-symmetric_UUA-GGC | AAUACAUCCACGGCGGCUG | | | | | | | | |
| | 0_1-1_mismatch_A-C | AAUUCGCGCUGACCACACU | | | | | | | | |
| | 26_6-6_internal_loop- | GUCGUCGAAUGGCCACUCCC | | | | | | | | |
| | symmetric_AAAGGC-UAAGGA | AGU | | | | | | | | |
| 2667 | −4_6-6_internal_loop- | GCCACAACUCCCUCCUCAUU | −4 | 28 | 0.288 | 0.849 | 0.846 | 1.04 | 1.249 | 1.285 |
| | symmetric_UCAUUA-GGCGAU | GAUUGAAAGUCCUUUCAUG | | | | | | | | |
| | 0_1-1_mismatch_A-C | AAUACCCCACGGCGGCGAU | | | | | | | | |
| | 4_2-1_bulge-asymmetric_AU-C | AUUCCUUUACACCACACUG | | | | | | | | |
| | 28_6-6_internal_loop- | UCGUCGAAUGGCCACUCCCA | | | | | | | | |
| | symmetric_AGGCCA-CAUUGA | GU | | | | | | | | |
| 2668 | −33_4-4_bulge-symmetric_UUCG- | GCCACACAGACCUCCUCAUU | −4 | 28 | 0.105 | 0.86 | 0.887 | 0.995 | 1.603 | 1.643 |
| | ACAU | GAUUGAAAGUCCUUUCAUG | | | | | | | | |
| | −20_4-4_bulge-symmetric_UGGU- | AAUACCCCACGGCGGCGAU | | | | | | | | |
| | UAGU | AUUCCUUUACUAGUCACUG | | | | | | | | |
| | −4_6-6_internal_loop- | UCGUACAUUGGCCACUCCCA | | | | | | | | |
| | symmetric_UCAUUA-GGCGAU | GU | | | | | | | | |
| | 0_1-1_mismatch_A-C | | | | | | | | | |
| | 4_2-1_bulge-asymmetric_AU-C | | | | | | | | | |
| | 28_6-6_internal_loop- | | | | | | | | | |
| | symmetric_AGGCCA-CAUUGA | | | | | | | | | |
| | 40_4-4_bulge-symmetric_GAGU- | | | | | | | | | |
| | CAGA | | | | | | | | | |
| 2669 | −10_6-6_internal_loop- | GCCACAACUCCCUCCUUGAU | −10 | 26 | 0.329 | 0.801 | 0.734 | 0.906 | 1.227 | 0.988 |
| | symmetric_AGGAAU-CGAUAG | AAUCGAAAGUCCUUUCAUG | | | | | | | | |
| | −5_2-2_bulge-symmetric_UU-CU | AAUACAUCCACGGCUCUUG | | | | | | | | |
| | 0_1-1_mismatch_A-C | ACGAUAGUUACACCACACU | | | | | | | | |
| | 26_6-6_internal_loop- | GUCGUCGAAUGGCCACUCCC | | | | | | | | |
| | symmetric_AAAGGC-AUAAUC | AGU | | | | | | | | |
| 2670 | −33_4-4_bulge-symmetric_UUCG- | GCCACACAGACCUCCUUGAU | −10 | 26 | 0.313 | 0.891 | 0.88 | 1.087 | 1.57 | 1.489 |
| | ACAU | AAUCGAAAGUCCUUUCAUG | | | | | | | | |
| | −20_4-4_bulge-symmetric_UGGU- | AAUACAUCCACGGCUCUUG | | | | | | | | |
| | UAGU | ACGAUAGUUACUAGUCACU | | | | | | | | |
| | −10_6-6_internal_loop- | GUCGUACAUUGGCCACUCCC | | | | | | | | |
| | symmetric_AGGAAU-CGAUAG | AGU | | | | | | | | |
| | −5_2-2_bulge-symmetric_UU-CU | | | | | | | | | |
| | 0_1-1_mismatch_A-C | | | | | | | | | |
| | 26_6-6_internal_loop- | | | | | | | | | |
| | symmetric_AAAGGC-AUAAUC | | | | | | | | | |
| | 40_4-4_bulge-symmetric_GAGU- | | | | | | | | | |
| | CAGA | | | | | | | | | |
| 2671 | −20_6-6_internal_loop- | GCCACAACUCCCUCCUUGAU | −20 | 26 | 0.216 | 0.769 | 0.758 | 0.962 | 1.216 | 1.186 |
| | symmetric_UGUGGU-UGGAGU | GGCGGAAAGUCCCUUCAUG | | | | | | | | |
| | −5_4-4_bulge-symmetric_CAUU- | AAUACUAUCCACGGCUCUA | | | | | | | | |
| | CUAC | CAAUUCCUUUACUGGAGUC | | | | | | | | |
| | 0_1-1_mismatch_A-C | UGUCGUCGAAUGGCCACUC | | | | | | | | |
| | 5_0-1_bulge-asymmetric_-U | CCAGU | | | | | | | | |
| | 17_1-1_mismatch_A-C | | | | | | | | | |
| | 26_6-6_internal_loop- | | | | | | | | | |
| | symmetric_AAAGGC-AUGGCG | | | | | | | | | |
| 2672 | −4_6-6_internal_loop- | GCCACAACUCCCUCCUCCAA | −4 | 28 | 0.254 | 0.798 | 0.77 | 0.965 | 1.25 | 1.219 |
| | symmetric_UCAUUA-GGUUAC | GAUUGAAAGUCCUUUCAAG | | | | | | | | |
| | 0_1-1_mismatch_A-C | AAUACAUCCACGGCGGUUA | | | | | | | | |
| | 12_1-1_mismatch_A-A | CAUUCCUUUACACCACACUG | | | | | | | | |
| | 28_6-6_internal_loop- | UCGUCGAAUGGCCACUCCCA | | | | | | | | |
| | symmetric_AGGCCA-CCAAGA | GU | | | | | | | | |
| 2673 | −33_4-4_bulge-symmetric_UUCG- | GCCACACAGACCUCCUCCAA | −4 | 28 | 0.065 | 0.908 | 0.905 | 0.949 | 1.64 | 1.649 |
| | ACAU | GAUUGAAAGUCCUUUCAAG | | | | | | | | |
| | −20_4-4_bulge-symmetric_UGGU- | AAUACAUCCACGGCGGUUA | | | | | | | | |
| | UAGU | CAUUCCUUUACUAGUCACU | | | | | | | | |
| | −4_6-6_internal_loop- | GUCGUACAUUGGCCACUCCC | | | | | | | | |
| | symmetric_UCAUUA-GGUUAC | AGU | | | | | | | | |
| | 0_1-1_mismatch_A-C | | | | | | | | | |
| | 12_1-1_mismatch_A-A | | | | | | | | | |
| | 28_6-6_internal_loop- | | | | | | | | | |
| | symmetric_AGGCCA-CCAAGA | | | | | | | | | |
| | 40_4-4_bulge-symmetric_GAGU- | | | | | | | | | |
| | CAGA | | | | | | | | | |
| 2674 | −4_6-6_internal_loop- | GCCACAACUCCCUCCUGACA | −4 | 28 | 0.377 | 0.84 | 0.821 | 1.029 | 1.255 | 1.35 |
| | symmetric_UCAUUA-GUGCAU | UAUUGAAAGUCCUUUCAUC | | | | | | | | |
| | 0_1-1_mismatch_A-C | AAUACAUCCACGGCGUGCA | | | | | | | | |
| | 11_1-1_mismatch_C-C | UAUUCCUUUACACCACACU | | | | | | | | |
| | 28_6-6_internal_loop- | GUCGUCGAAUGGCCACUCCC | | | | | | | | |
| | symmetric_AGGCCA-GACAUA | AGU | | | | | | | | |
| 2675 | −33_4-4_bulge-symmetric_UUCG- | GCCACACAGACCUCCUGACA | −4 | 28 | 0.299 | 0.904 | 0.915 | 1.154 | 1.613 | 1.568 |
| | ACAU | UAUUGAAAGUCCUUUCAUC | | | | | | | | |
| | −20_4-4_bulge-symmetric_UGGU- | AAUACAUCCACGGCGUGCA | | | | | | | | |
| | UAGU | UAUUCCUUUACUAGUCACU | | | | | | | | |
| | −4_6-6_internal_loop- | GUCGUACAUUGGCCACUCCC | | | | | | | | |
| | symmetric_UCAUUA-GUGCAU | AGU | | | | | | | | |
| | 0_1-1_mismatch_A-C | | | | | | | | | |
| | 11_1-1_mismatch_C-C | | | | | | | | | |
| | 28_6-6_internal_loop- | | | | | | | | | |
| | symmetric_AGGCCA-GACAUA | | | | | | | | | |
| | 40_4-4_bulge-symmetric_GAGU- | | | | | | | | | |
| | CAGA | | | | | | | | | |
| 2676 | −6_6-6_internal_loop- | GCCACAACUCCCUCCUGAUA | −6 | 28 | 0.316 | 0.815 | 0.761 | 1.047 | 1.302 | 1.283 |
| | symmetric_AUUCAU-UUCAUC | UGUUGAAAGUCCUUUCCUG | | | | | | | | |
| | 0_1-1_mismatch_A-C | AAUACAUUCACGGCUAUUC | | | | | | | | |
| | 3_1-1_wobble_G-U | AUCUCCUUUACACCACACUG | | | | | | | | |
| | 13_1-1_mismatch_U-C | UCGUCGAAUGGCCACUCCCA | | | | | | | | |
| | 28_6-6_internal_loop- | GU | | | | | | | | |
| | symmetric_AGGCCA-GAUAUG | | | | | | | | | |
| 2677 | −33_4-4_bulge-symmetric_UUCG- | GCCACACAGACCUCCUGAUA | −6 | 28 | 0.09 | 0.894 | 0.886 | 0.952 | 1.644 | 1.628 |
| | ACAU | UGUUGAAAGUCCUUUCCUG | | | | | | | | |
| | −20_4-4_bulge-symmetric_UGGU- | AAUACAUUCACGGCUAUUC | | | | | | | | |
| | UAGU | AUCUCCUUUACUAGUCACU | | | | | | | | |
| | −6_6-6_internal_loop- | GUCGUACAUUGGCCACUCCC | | | | | | | | |
| | symmetric_AUUCAU-UUCAUC | AGU | | | | | | | | |
| | 0_1-1_mismatch_A-C | | | | | | | | | |
| | 3_1-1_wobble_G-U | | | | | | | | | |
| | 13_1-1_mismatch_U-C | | | | | | | | | |
| | 28_6-6_internal_loop- | | | | | | | | | |
| | symmetric_AGGCCA-GAUAUG | | | | | | | | | |
| | 40_4-4_bulge-symmetric_GAGU- | | | | | | | | | |
| | CAGA | | | | | | | | | |
| 2678 | −8_6-6_internal_loop- | GCCACAACUCCCUCCUCUAU | −8 | 28 | 0.372 | 0.815 | 0.771 | 1.034 | 1.293 | 1.133 |
| | symmetric_GAAUUC-UGUUGG | UAUUGAAAGUCCUUUCAUU | | | | | | | | |
| | 0_1-1_mismatch_A-C | AAUACAUCCACGGCUAAUU | | | | | | | | |
| | 11_1-1_mismatch_C-U | GUUGGCUUUACACCACACU | | | | | | | | |
| | 28_6-6_internal_loop- | GUCGUCGAAUGGCCACUCCC | | | | | | | | |
| | symmetric_AGGCCA-CUAUUA | AGU | | | | | | | | |
| 2679 | −33_4-4_bulge-symmetric_UUCG- | GCCACACAGACCUCCUCUAU | −8 | 28 | 0.292 | 0.912 | 0.909 | 1.128 | 1.642 | 1.666 |
| | ACAU | UAUUGAAAGUCCUUUCAUU | | | | | | | | |
| | −20_4-4_bulge-symmetric_UGGU- | AAUACAUCCACGGCUAAUU | | | | | | | | |
| | UAGU | GUUGGCUUUACUAGUCACU | | | | | | | | |
| | −8_6-6_internal_loop- | GUCGUACAUUGGCCACUCCC | | | | | | | | |
| | symmetric_GAAUUC-UGUUGG | AGU | | | | | | | | |
| | 0_1-1_mismatch_A-C | | | | | | | | | |
| | 11_1-1_mismatch_C-U | | | | | | | | | |
| | 28_6-6_internal_loop- | | | | | | | | | |
| | symmetric_AGGCCA-CUAUUA | | | | | | | | | |
| | 40_4-4_bulge-symmetric_GAGU- | | | | | | | | | |
| | CAGA | | | | | | | | | |
| 2680 | −14_6-6_internal_loop- | GCCACAACUCCCUCCUAACA | −14 | 28 | 0.261 | 0.769 | 0.725 | 1.023 | 1.16 | 1.289 |
| | symmetric_GUAAAG-GGUCCG | UCUUGAAAGUCCUUUCAUG | | | | | | | | |
| | −5_3-3_bulge-symmetric_AUU-UUG | AAUACAUCCACGGCUUUGG | | | | | | | | |
| | 0_1-1_mismatch_A-C | AAUUCGGUCCGACCACACU | | | | | | | | |
| | 28_6-6_internal_loop- | GUCGUCGAAUGGCCACUCCC | | | | | | | | |
| | symmetric_AGGCCA-AACAUC | AGU | | | | | | | | |
| 2681 | −10_6-6_internal_loop- | GCCACAACUCCCUCCUCUCA | −10 | 28 | 0.317 | 0.785 | 0.701 | 1.123 | 1.242 | 1.188 |
| | symmetric_AGGAAU-UGCAUA | GGUUGAAAGUCCUUUCAUG | | | | | | | | |
| | −5_3-3_bulge-symmetric_AUU-UCC | GAUACAUCCACGGCUUCCG | | | | | | | | |
| | 0_1-1_mismatch_A-C | AUGCAUAUUACACCACACU | | | | | | | | |
| | 10_1-1_wobble_U-G | GUCGUCGAAUGGCCACUCCC | | | | | | | | |
| | 28_6-6_internal_loop- | AGU | | | | | | | | |
| | symmetric_AGGCCA-CUCAGG | | | | | | | | | |

### Example 19

### Engineered Guide RNAs Targeting LRRK2

This example describes the on-target and off-target editing efficiencies of various engineered guide RNAs disclosed herein targeting the LRRK2 G2019S mutation via ADAR. A side-by-side comparison of engineered guide RNAs with varying structural features in the guide-target RNA scaffold that forms upon hybridization of the engineered guide RNA to a target LRRK2 mRNA was assessed in cells (also referred to herein as “in trans”). Engineered guide RNAs were dosed in vitro in cells expressing ADAR1. RNA editing at the on-target adenosine and at local off-target adenosines was assessed by Sanger sequencing.

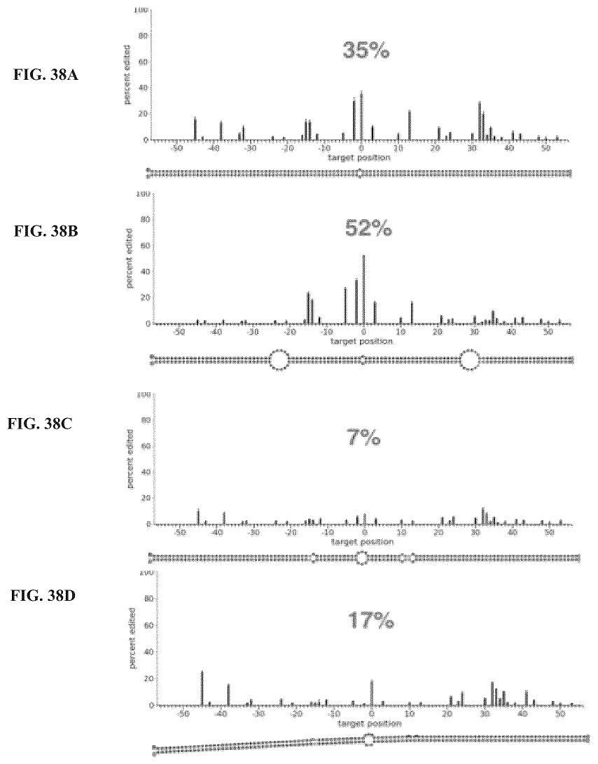

FIG. 38A-FIG. 38D show the editing profiles of four engineered guide RNAs via ADAR, including an engineered guide RNA that forms an A/C mismatch structural feature at the target adenosine (FIG. 38A), an engineered guide RNA that forms an A/C mismatch structural feature at the target adenosine and barbells at the −20 and +26 positions relative to the target adenosine (position 0) (FIG. 38B), an engineered guide RNA that forms A/G mismatch structural features at local off-target adenosines at the −14, −2, +10, and +13 positions relative to the target adenosine (position 0) (FIG. 38C), and an engineered guide RNA that forms a 1/0 asymmetrical bulge at local off-target adenosines at the −14, −2, +10, and +13 positions relative to the target adenosine (position 0) by deletion of a U opposite the local off-target adenosine (FIG. 38D). The plots shown in FIG. 38A-FIG. 38D depict the position along the target RNA on the x-axis and report percent editing on the y-axis. A diagram of the guide-target RNA scaffold for each engineered guide RNA is shown directly below each graph. As shown in FIG. 38A-FIG. 38D, simple addition of A/G mismatches or 1/0 bulges at local off-target adenosines resulted in abrogated on-target editing as compared to the A/C mismatch engineered guide RNA. The addition of a barbell macro-footprint resulted in increase of on-target editing as compared to the A/C mismatch engineered guide RNA.

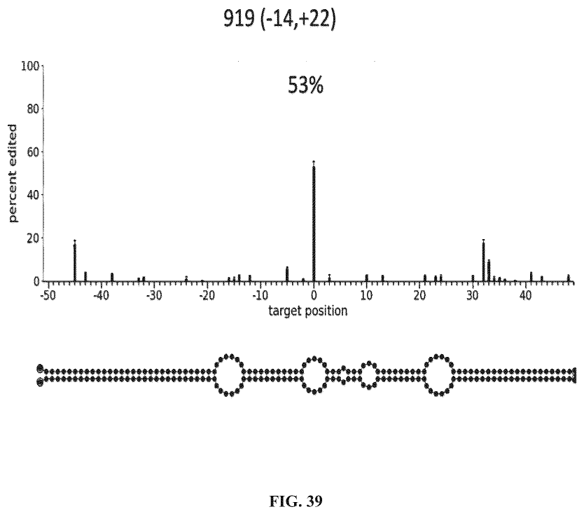

An engineered guide RNA that forms a barbell macro-footprint at the −14 and +22 positions relative to the target adenosine (position 0) and which also forms a micro-footprint comprising other structural features was also evaluated in cells (FIG. 39). This engineered guide RNA displayed a boost in on-target adenosine editing and a reduction in local off-target adenosine editing as compared to the A/C mismatch engineered guide RNA.

### Example 20

### Engineering of Latent Structures of the Guide-Target RNA Scaffold to Minimize +1 Editing of ABCA4 G5882A Nucleotide Mutation for ABCA4 Resulting in a G1961E Amino Acid Mutation

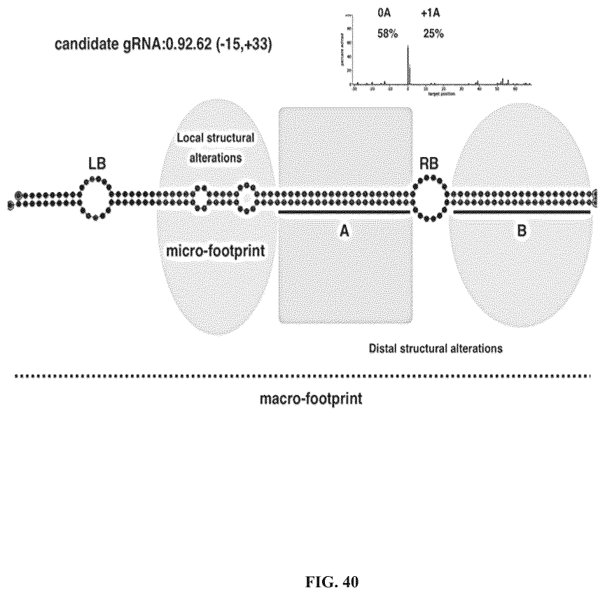

This example describes engineering of latent structures of the guide-target RNA complex to minimize editing at the +1 position, relative to the target A (targeting the G5882A nucleotide mutation resulting in the G1961E amino acid mutation), in target ABCA4 RNA. As shown in FIG. 40, a candidate engineered guide RNA when bound to a target ABCA4 mRNA forms a guide-target RNA scaffold having a micro-footprint, and a macrofootprint comprising a right barbell and a left barbell. As shown in the RNA editing profile (top right of FIG. 40), the candidate engineered guide RNA facilitates 58% on target adenosine editing and 25% off-target editing at the adenosine at the +1 position (“+1 editing”). This example demonstrates that tiling of latent structures (here symmetrical loops and bulges) within the region (A) shown in FIG. 40 minimizes the amount of +1 editing relative to the target adenosine (“position 0”).

The following engineered guide RNAs recited in TABLE 8 were each used in this experiment. While the engineered guide RNA sequences in TABLE 8 are provided as DNA sequences with a T substituted for each U, the corresponding RNA sequences are also encompassed herein.

TABLE 8
ABCA4 Engineered Guide RNA Sequences

| | | SEQ | | | | |
|---|---|---|---|---|---|---|
| | | ID | | | | |
| FIG. | Guide Name | NO | Sequence | Structural Features(target/guide) | LB | RB |
| 41 | 0.92.62(−15, +33) | 2772 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 33_6- | | |
| | | | CTGGGCTGGA | 6_internal_loop-symmetric_GAGUGA-AGUGAG | | |
| 41 | 0.92.62(−15, +33)+4(6/6) | 2773 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCTGAGAGCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_12_6- | | |
| | | | CTGGGCTGGA | 6_internal_loop-symmetric_GAGAGU-UGAGAG, 33_6- | | |
| | | | | 6_internal_loop-symmetric_GAGUGA-AGUGAG | | |
| 41 | 0.92.62(−15, +33)+5(6/6) | 2774 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGGTGAGACCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_13_6- | | |
| | | | CTGGGCTGGA | 6_internal_loop-symmetric_AGAGUG-GUGAGA, 33_6- | | |
| | | | | 6_internal_loop-symmetric_GAGUGA-AGUGAG | | |
| 41 | 0.92.62(−15, +33)+6(6/6) | 2775 | TGTGGTTGTTTTGCCGGCACCATAGTGA | −15_6-5_internal_loop-asymmetric_AGUGGA-AGUGA, | −15 | 33 |
| | | | GCCAGGAGGCCAAATTTGAGTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_14_6- | | |
| | | | CTGGGCTGGA | 6_internal_loop-symmetric_GAGUGC-UUUGAG, 33_6- | | |
| | | | | 6_internal_loop-symmetric_GAGUGA-AGUGAG | | |
| 41 | 0.92.62(−15, +33)+7(6/6) | 2776 | TGTGGTTGTTTTGCCGGCACCATAGTGA | −15_6-5_internal_loop-asymmetric_AGUGGA-AGUGA, | −15 | 33 |
| | | | GCCAGGAGGCCAATTTTGACTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_15_6- | | |
| | | | TGGGCTGGA | 6_internal_loop-symmetric_AGUGCU-UUUUGA, 33_6- | | |
| | | | | 6_internal_loop-symmetric_GAGUGA-AGUGAG | | |
| 41 | 0.92.62(−15, +33)+8(6/6) | 2777 | TGTGGTTGTTTTGCCGGCACCATAGTGA | −15_6-5_internal_loop-asymmetric_AGUGGA-AGUGA, | −15 | 33 |
| | | | GCCAGGAGGCCATTTTTGTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_16_6- | | |
| | | | TGGGCTGGA | 6_internal_loop-symmetric_GUGCUU-UUUUUG, 33_6- | | |
| | | | | 6_internal_loop-symmetric_GAGUGA-AGUGAG | | |
| 41 | 0.92.62(−15, +33)+9(6/6) | 2778 | TGTGGTTGTTTTGCCGGCACCATAGTGA | −15_6-5_internal_loop-asymmetric_AGUGGA-AGUGA, | −15 | 33 |
| | | | GCCAGGAGGCCTTTTTTCTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_17_6- | | |
| | | | TGGGCTGGA | 6_internal_loop-symmetric_UGCUUU-UUUUUU, 33_6- | | |
| | | | | 6_internal_loop-symmetric_GAGUGA-AGUGAG | | |
| 41 | 0.92.62(−15, +33)+10(6/6) | 2779 | TGTGGTTGTTTTGCCGGCACCATAGTGA | −15_6-5_internal_loop-asymmetric_AGUGGA-AGUGA, | −15 | 33 |
| | | | GCCAGGAGGCGTTTTAACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_18_6- | | |
| | | | TGGGCTGGA | 6_internal_loop-symmetric_GCUUUG-GUUUUA, 33_6- | | |
| | | | | 6_internal_loop-symmetric_GAGUGA-AGUGAG | | |
| 41 | 0.92.62(−15, +33)+11(6/6) | 2780 | TGTGGTTGTTTTGCCGGCACCATAGTGA | −15_6-5_internal_loop-asymmetric_AGUGGA-AGUGA, | −15 | 33 |
| | | | GCCAGGAGGGGTTTTCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_19_6- | | |
| | | | TGGGCTGGA | 6_internal_loop-symmetric_CUUUGG-GGUUUU, 33_6- | | |
| | | | | 6_internal_loop-symmetric_GAGUGA-AGUGAG | | |
| 41 | 0.92.62(−15, +33)+12(6/6) | 2781 | TGTGGTTGTTTTGCCGGCACCATAGTGA | −15_6-5_internal_loop-asymmetric_AGUGGA-AGUGA, | −15 | 33 |
| | | | GCCAGGAGTAGGTTGCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U, 20_6- | | |
| | | | TGGGCTGGA | 6_internal_loop-symmetric_UUUGGC-UAGGUU, 33_6- | | |
| | | | | 6_internal_loop-symmetric_GAGUGA-AGUGAG | | |
| 41 | 0.92.62(−15, +33)+13(6/6) | 2782 | TGTGGTTGTTTTGCCGGCACCATAGTGA | −15_6-5_internal_loop-asymmetric_AGUGGA-AGUGA, | −15 | 33 |
| | | | GCCAGGATTTTTTAGCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U, 21_6- | | |
| | | | TGGGCTGGA | 6_internal_loop-symmetric_UUGGCC-UUUUUU, 33_6- | | |
| | | | | 6_internal_loop-symmetric_GAGUGA-AGUGAG | | |
| 41 | 0.92.62(−15, +33)+14(6/6) | 2783 | TGTGGTTGTTTTGCCGGCACCATAGTGA | −15_6-5_internal_loop-asymmetric_AGUGGA-AGUGA, | −15 | 33 |
| | | | GCCAGGTTTTTTAAGCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U, 22_6- | | |
| | | | TGGGCTGGA | 6_internal_loop-symmetric_UGGCCU-UUUUUU, 33_6- | | |
| | | | | 6_internal_loop-symmetric_GAGUGA-AGUGAG | | |
| 41 | 0.92.62(−15, +33)+4(5/5) | 2784 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCAGAGAGCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_12_5- | | |
| | | | CTGGGCTGGA | 5_internal_loop-symmetric_GAGAG-GAGAG, 33_6- | | |
| | | | | 6_internal_loop-symmetric_GAGUGA-AGUGAG | | |
| 41 | 0.92.62(−15, +33)+5(5/5) | 2785 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCTGAGACCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_13_5- | | |
| | | | CTGGGCTGGA | 5_internal_loop-symmetric_AGAGU-UGAGA, 33_6- | | |
| | | | | 6_internal_loop-symmetric_GAGUGA-AGUGAG | | |
| 41 | 0.92.62(−15, +33)+6(5/5) | 2786 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGGTGAGTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_14_5- | | |
| | | | CTGGGCTGGA | 5_internal_loop-symmetric_GAGUG-GUGAG, 33_6- | | |
| | | | | 6_internal_loop-symmetric_GAGUGA-AGUGAG | | |
| 41 | 0.92.62(−15, +33)+7(5/5) | 2787 | TGTGGTTGTTTTGCCGGCACCATAGTGA | −15_6-5_internal_loop-asymmetric_AGUGGA-AGUGA, | −15 | 33 |
| | | | GCCAGGAGGCCAAATTTGACTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_15_5- | | |
| | | | CTGGGCTGGA | 5_internal_loop-symmetric_AGUGC-UUUGA, 33_6- | | |
| | | | | 6_internal_loop-symmetric_GAGUGA-AGUGAG | | |
| 41 | 0.92.62(−15, +33)+8(5/5) | 2788 | TGTGGTTGTTTTGCCGGCACCATAGTGA | −15_6-5_internal_loop-asymmetric_AGUGGA-AGUGA, | −15 | 33 |
| | | | GCCAGGAGGCCAATTTTGTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_16_5- | | |
| | | | TGGGCTGGA | 5_internal_loop-symmetric_GUGCU-UUUUG, 33_6- | | |
| | | | | 6_internal_loop-symmetric_GAGUGA-AGUGAG | | |
| 41 | 0.92.62(−15, +33)+9(5/5) | 2789 | TGTGGTTGTTTTGCCGGCACCATAGTGA | −15_6-5_internal_loop-asymmetric_AGUGGA-AGUGA, | −15 | 33 |
| | | | GCCAGGAGGCCATTTTTCTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_17_5- | | |
| | | | TGGGCTGGA | 5_internal_loop-symmetric_UGCUU-UUUUU, 33_6- | | |
| | | | | 6_internal_loop-symmetric_GAGUGA-AGUGAG | | |
| 41 | 0.92.62(−15, +33)+10(5/5) | 2790 | TGTGGTTGTTTTGCCGGCACCATAGTGA | −15_6-5_internal_loop-asymmetric_AGUGGA-AGUGA, | −15 | 33 |
| | | | GCCAGGAGGCCTTTTAACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_18_5- | | |
| | | | TGGGCTGGA | 5_internal_loop-symmetric_GCUUU-UUUUA, 33_6- | | |
| | | | | 6_internal_loop-symmetric_GAGUGA-AGUGAG | | |
| 41 | 0.92.62(−15, +33)+11(5/5) | 2791 | TGTGGTTGTTTTGCCGGCACCATAGTGA | −15_6-5_internal_loop-asymmetric_AGUGGA-AGUGA, | −15 | 33 |
| | | | GCCAGGAGGCGTTTCCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_19_5- | | |
| | | | TGGGCTGGA | 5_internal_loop-symmetric_CUUUG-GUUUC, 33_6- | | |
| | | | | 6_internal_loop-symmetric_GAGUGA-AGUGAG | | |
| 41 | 0.92.62(−15, +33)+12(5/5) | 2792 | TGTGGTTGTTTTGCCGGCACCATAGTGA | −15_6-5_internal_loop-asymmetric_AGUGGA-AGUGA, | −15 | 33 |
| | | | GCCAGGAGGGGTTTGCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U, 20_5- | | |
| | | | TGGGCTGGA | 5_internal_loop-symmetric_UUUGG-GGUUU, 33_6- | | |
| | | | | 6_internal_loop-symmetric_GAGUGA-AGUGAG | | |
| 41 | 0.92.62(−15, +33)+13(5/5) | 2793 | TGTGGTTGTTTTGCCGGCACCATAGTGA | −15_6-5_internal_loop-asymmetric_AGUGGA-AGUGA, | −15 | 33 |
| | | | GCCAGGAGAATTTAGCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U, 21_5- | | |
| | | | TGGGCTGGA | 5_internal_loop-symmetric_UUGGC-AAUUU, 33_6- | | |
| | | | | 6_internal_loop-symmetric_GAGUGA-AGUGAG | | |
| 41 | 0.92.62(−15, +33)+14(5/5) | 2794 | TGTGGTTGTTTTGCCGGCACCATAGTGA | −15_6-5_internal_loop-asymmetric_AGUGGA-AGUGA, | −15 | 33 |
| | | | GCCAGGAAAATTAAGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 22_5- | | |
| | | | CTGGGCTGGA | 5_internal_loop-symmetric_UGGCC-AAAUU, 33_6- | | |
| | | | | 6_internal_loop-symmetric_GAGUGA-AGUGAG | | |
| 41 | 0.92.62(−15, +33)+15(5/5) | 2795 | TGTGGTTGTTTTGCCGGCACCATAGTGA | −15_6-5_internal_loop-asymmetric_AGUGGA-AGUGA, | −15 | 33 |
| | | | GCCAGGTTTAAAAAGCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U, 23_5- | | |
| | | | TGGGCTGGA | 5_internal_loop-symmetric_GGCCU-UUUAA, 33_6- | | |
| | | | | 6_internal_loop-symmetric_GAGUGA-AGUGAG | | |
| 42 | 0.92.62(−15, +33)+4(4/4) | 2796 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCACAGAGCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_12_4-4_bulge- | | |
| | | | CTGGGCTGGA | symmetric_GAGA-AGAG, 33_6-6_internal_loop- | | |
| | | | | symmetric_GAGUGA-AGUGAG | | |
| 42 | 0.92.62(−15, +33)+5(4/4) | 2797 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCAGAGACCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_13_4-4_bulge- | | |
| | | | CTGGGCTGGA | symmetric_AGAG-GAGA, 33_6-6_internal_loop- | | |
| | | | | symmetric_GAGUGA-AGUGAG | | |
| 42 | 0.92.62(−15, +33)+6(4/4) | 2798 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCTGAGTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_14_4-4_bulge- | | |
| | | | CTGGGCTGGA | symmetric_GAGU-UGAG, 33_6-6_internal_loop- | | |
| | | | | symmetric_GAGUGA-AGUGAG | | |
| 42 | 0.92.62(−15, +33)+7(4/4) | 2799 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGGTGACTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_15_4-4_bulge- | | |
| | | | CTGGGCTGGA | symmetric_AGUG-GUGA, 33_6-6_internal_loop- | | |
| | | | | symmetric_GAGUGA-AGUGAG | | |
| 42 | 0.92.62(−15, +33)+8(4/4) | 2800 | TGTGGTTGTTTTGCCGGCACCATAGTGA | −15_6-5_internal_loop-asymmetric_AGUGGA-AGUGA, | −15 | 33 |
| | | | GCCAGGAGGCCAAATTTGTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_16_4-4_bulge- | | |
| | | | TGGGCTGGA | symmetric_GUGC-UUUG, 33_6-6_internal_loop- | | |
| | | | | symmetric_GAGUGA-AGUGAG | | |
| 42 | 0.92.62(−15, +33)+9(4/4) | 2801 | TGTGGTTGTTTTGCCGGCACCATAGTGA | −15_6-5_internal_loop-asymmetric_AGUGGA-AGUGA, | −15 | 33 |
| | | | GCCAGGAGGCCAATTTTCTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_17_4-4_bulge- | | |
| | | | TGGGCTGGA | symmetric_UGCU-UUUU, 33_6-6_internal_loop- | | |
| | | | | symmetric_GAGUGA-AGUGAG | | |
| 42 | 0.92.62(−15, +33)+10(4/4) | 2802 | TGTGGTTGTTTTGCCGGCACCATAGTGA | −15_6-5_internal_loop-asymmetric_AGUGGA-AGUGA, | −15 | 33 |
| | | | GCCAGGAGGCCATTTAACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_18_4-4_bulge- | | |
| | | | TGGGCTGGA | symmetric_GCUU-UUUA, 33_6-6_internal_loop- | | |
| | | | | symmetric_GAGUGA-AGUGAG | | |
| 42 | 0.92.62(−15, +33)+11(4/4) | 2803 | TGTGGTTGTTTTGCCGGCACCATAGTGA | −15_6-5_internal_loop-asymmetric_AGUGGA-AGUGA, | −15 | 33 |
| | | | GCCAGGAGGCCTTTCCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_19_4-4_bulge- | | |
| | | | TGGGCTGGA | symmetric_CUUU-UUUC, 33_6-6_internal_loop- | | |
| | | | | symmetric_GAGUGA-AGUGAG | | |
| 42 | 0.92.62(−15, +33)+12(4/4) | 2804 | TGTGGTTGTTTTGCCGGCACCATAGTGA | −15_6-5_internal_loop-asymmetric_AGUGGA-AGUGA, | −15 | 33 |
| | | | GCCAGGAGGCGTTTGCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U, 20_4-4_bulge- | | |
| | | | TGGGCTGGA | symmetric_UUUG-GUUU, 33_6-6_internal_loop- | | |
| | | | | symmetric_GAGUGA-AGUGAG | | |
| 42 | 0.92.62(−15, +33)+13(4/4) | 2805 | TGTGGTTGTTTTGCCGGCACCATAGTGA | −15_6-5_internal_loop-asymmetric_AGUGGA-AGUGA, | −15 | 33 |
| | | | GCCAGGAGGATTTAGCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U, 21_4-4_bulge- | | |
| | | | TGGGCTGGA | symmetric_UUGG-AUUU, 33_6-6_internal_loop- | | |
| | | | | symmetric_GAGUGA-AGUGAG | | |
| 42 | 0.92.62(−15, +33)+14(4/4) | 2806 | TGTGGTTGTTTTGCCGGCACCATAGTGA | −15_6-5_internal_loop-asymmetric_AGUGGA-AGUGA, | −15 | 33 |
| | | | GCCAGGAGAATTAAGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 22_4-4_bulge- | | |
| | | | CTGGGCTGGAG | symmetric_UGGC-AAUU, 33_6-6_internal_loop- | | |
| | | | | symmetric_GAGUGA-AGUGAG | | |
| 42 | 0.92.62(−15, +33)+15(4/4) | 2807 | TGTGGTTGTTTTGCCGGCACCATAGTGA | −15_6-5_internal_loop-asymmetric_AGUGGA-AGUGA, | −15 | 33 |
| | | | GCCAGGATTAAAAAGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 23_4-4_bulge- | | |
| | | | CTGGGCTGGA | symmetric_GGCC-UUAA, 33_6-6_internal_loop- | | |
| | | | | symmetric_GAGUGA-AGUGAG | | |
| 42 | 0.92.62(−15, +33)+4(3/3) | 2808 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCACTGAGCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_12_3-3_bulge- | | |
| | | | CTGGGCTGGA | symmetric_GAG-GAG, 33_6-6_internal_loop- | | |
| | | | | symmetric_GAGUGA-AGUGAG | | |
| 42 | 0.92.62(−15, +33)+5(3/3) | 2809 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCACAGACCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_13_3-3_bulge- | | |
| | | | CTGGGCTGGA | symmetric_AGA-AGA, 33_6-6_internal_loop- | | |
| | | | | symmetric_GAGUGA-AGUGAG | | |
| 42 | 0.92.62(−15, +33)+6(3/3) | 2810 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCAGAGTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_14_3-3_bulge- | | |
| | | | CTGGGCTGGA | symmetric_GAG-GAG, 33_6-6_internal_loop- | | |
| | | | | symmetric_GAGUGA-AGUGAG | | |
| 42 | 0.92.62(−15, +33)+7(3/3) | 2811 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCTGACTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_15_3-3_bulge- | | |
| | | | CTGGGCTGGA | symmetric_AGU-UGA, 33_6-6_internal_loop- | | |
| | | | | symmetric_GAGUGA-AGUGAG | | |
| 42 | 0.92.62(−15, +33)+8(3/3) | 2812 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGGTGTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_16_3-3_bulge- | | |
| | | | CTGGGCTGGA | symmetric_GUG-GUG, 33_6-6_internal_loop- | | |
| | | | | symmetric_GAGUGA-AGUGAG | | |
| 42 | 0.92.62(−15, +33)+9(3/3) | 2813 | TGTGGTTGTTTTGCCGGCACCATAGTGA | −15_6-5_internal_loop-asymmetric_AGUGGA-AGUGA, | −15 | 33 |
| | | | GCCAGGAGGCCAAATTTCTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_17_3-3_bulge- | | |
| | | | TGGGCTGGA | symmetric_UGC-UUU, 33_6-6_internal_loop- | | |
| | | | | symmetric_GAGUGA-AGUGAG | | |
| 42 | 0.92.62(−15, +33)+10(3/3) | 2814 | TGTGGTTGTTTTGCCGGCACCATAGTGA | −15_6-5_internal_loop-asymmetric_AGUGGA-AGUGA, | −15 | 33 |
| | | | GCCAGGAGGCCAATTAACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_18_3-3_bulge- | | |
| | | | CTGGGCTGGA | symmetric_GCU-UUA, 33_6-6_internal_loop- | | |
| | | | | symmetric_GAGUGA-AGUGAG | | |
| 42 | 0.92.62(−15, +33)+11(3/3) | 2815 | TGTGGTTGTTTTGCCGGCACCATAGTGA | −15_6-5_internal_loop-asymmetric_AGUGGA-AGUGA, | −15 | 33 |
| | | | GCCAGGAGGCCATTCCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U,_19_3-3_bulge- | | |
| | | | TGGGCTGGA | symmetric_CUU-UUC, 33_6-6_internal_loop- | | |
| | | | | symmetric_GAGUGA-AGUGAG | | |
| 42 | 0.92.62(−15, +33)+12(3/3) | 2816 | TGTGGTTGTTTTGCCGGCACCATAGTGA | −15_6-5_internal_loop-asymmetric_AGUGGA-AGUGA, | −15 | 33 |
| | | | GCCAGGAGGCCTTTGCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U, 20_3-3_bulge- | | |
| | | | TGGGCTGGA | symmetric_UUU-UUU, 33_6-6_internal_loop- | | |
| | | | | symmetric_GAGUGA-AGUGAG | | |
| 42 | 0.92.62(−15, +33)+13(3/3) | 2817 | TGTGGTTGTTTTGCCGGCACCATAGTGA | −15_6-5_internal_loop-asymmetric_AGUGGA-AGUGA, | −15 | 33 |
| | | | GCCAGGAGGCGTTAGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 21_3-3_bulge- | | |
| | | | CTGGGCTGGA | symmetric_UUG-GUU, 33_6-6_internal_loop- | | |
| | | | | symmetric_GAGUGA-AGUGAG | | |
| 42 | 0.92.62(−15, +33)+14(3/3) | 2818 | TGTGGTTGTTTTGCCGGCACCATAGTGA | −15_6-5_internal_loop-asymmetric_AGUGGA-AGUGA, | −15 | 33 |
| | | | GCCAGGAGGATTAAGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 22_3-3_bulge- | | |
| | | | CTGGGCTGGA | symmetric_UGG-AUU, 33_6-6_internal_loop- | | |
| | | | | symmetric_GAGUGA-AGUGAG | | |
| 42 | 0.92.62(−15, +33)+15(3/3) | 2819 | TGTGGTTGTTTTGCCGGCACCATAGTGA | −15_6-5_internal_loop-asymmetric_AGUGGA-AGUGA, | −15 | 33 |
| | | | GCCAGGAGTAAAAAGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 23_3-3_bulge- | | |
| | | | CTGGGCTGGA | symmetric_GGC-UAA, 33_6-6_internal_loop- | | |
| | | | | symmetric_GAGUGA-AGUGAG | | |
| 42 | 0.92.62(−15, +33)+4(2/2) | 2820 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCACTCAGCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_12_2-2_bulge- | | |
| | | | CTGGGCTGGA | symmetric_GA-AG, 33_6-6_internal_loop- | | |
| | | | | symmetric_GAGUGA-AGUGAG | | |
| 42 | 0.92.62(−15, +33)+5(2/2) | 2821 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCACTGACCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_13_2-2_bulge- | | |
| | | | CTGGGCTGGA | symmetric_AG-GA, 33_6-6_internal_loop- | | |
| | | | | symmetric_GAGUGA-AGUGAG | | |
| 42 | 0.92.62(−15, +33)+6(2/2) | 2822 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCACAGTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_14_2-2_bulge- | | |
| | | | CTGGGCTGGA | symmetric_GA-AG, 33_6-6_internal_loop- | | |
| | | | | symmetric_GAGUGA-AGUGAG | | |
| 42 | 0.92.62(−15, +33)+7(2/2) | 2823 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCAGACTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_15_2-2_bulge- | | |
| | | | CTGGGCTGGA | symmetric_AG-GA, 33_6-6_internal_loop- | | |
| | | | | symmetric_GAGUGA-AGUGAG | | |
| 42 | 0.92.62(−15, +33)+8(2/2) | 2824 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCTGTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_16_2-2_bulge- | | |
| | | | CTGGGCTGGA | symmetric_GU-UG, 33_6-6_internal_loop- | | |
| | | | | symmetric_GAGUGA-AGUGAG | | |
| 42 | 0.92.62(−15, +33)+9(2/2) | 2825 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGATCTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_17_2-2_bulge- | | |
| | | | CTGGGCTGGA | symmetric_UG-AU, 33_6-6_internal_loop- | | |
| | | | | symmetric_GAGUGA-AGUGAG | | |
| 42 | 0.92.62(−15, +33)+10(2/2) | 2826 | TGTGGTTGTTTTGCCGGCACCATAGTGA | −15_6-5_internal_loop-asymmetric_AGUGGA-AGUGA, | −15 | 33 |
| | | | GCCAGGAGGCCAAATAACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_18_2-2_bulge- | | |
| | | | CTGGGCTGGA | symmetric_GC-UA, 33_6-6_internal_loop- | | |
| | | | | symmetric_GAGUGA-AGUGAG | | |
| 42 | 0.92.62(−15, +33)+11(2/2) | 2827 | TGTGGTTGTTTTGCCGGCACCATAGTGA | −15_6-5_internal_loop-asymmetric_AGUGGA-AGUGA, | −15 | 33 |
| | | | GCCAGGAGGCCAATCCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_19_2-2_bulge- | | |
| | | | CTGGGCTGGA | symmetric_CU-UC, 33_6-6_internal_loop- | | |
| | | | | symmetric_GAGUGA-AGUGAG | | |
| 42 | 0.92.62(−15, +33)+12(2/2) | 2828 | TGTGGTTGTTTTGCCGGCACCATAGTGA | −15_6-5_internal_loop-asymmetric_AGUGGA-AGUGA, | −15 | 33 |
| | | | GCCAGGAGGCCATTGCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U, 20_2-2_bulge- | | |
| | | | TGGGCTGGA | symmetric_UU-UU, 33_6-6_internal_loop- | | |
| | | | | symmetric_GAGUGA-AGUGAG | | |
| 42 | 0.92.62(−15, +33)+13(2/2) | 2829 | TGTGGTTGTTTTGCCGGCACCATAGTGA | −15_6-5_internal_loop-asymmetric_AGUGGA-AGUGA, | −15 | 33 |
| | | | GCCAGGAGGCCTTAGCACTCTCCAGTGA | −1->0_2-2_bulge-symmetric_GA-CG, 6_1- | | |
| | | | GAACTCGGACACACAGCCTGAGTGAGC | 1_mismatch_G-A, 8_1-1_mismatch_C-U, 21_2-2_bulge- | | |
| | | | TGGGCTGGA | symmetric_UU-UU, 33_6-6_internal_loop- | | |
| | | | | symmetric_GAGUGA-AGUGAG | | |
| 42 | 0.92.62(−15, +33)+14(2/2) | 2830 | TGTGGTTGTTTTGCCGGCACCATAGTGA | −15_6-5_internal_loop-asymmetric_AGUGGA-AGUGA, | −15 | 33 |
| | | | GCCAGGAGGTGTAAGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 22_2-2_bulge- | | |
| | | | CTGGGCTGGA | symmetric_UG-GU, 24_1-1_wobble G-U, 33_6- | | |
| | | | | 6_internal_loop-symmetric_GAGUGA-AGUGAG | | |
| 42 | 0.92.62(−15, +33)+15(2/2) | 2831 | TGTGGTTGTTTTGCCGGCACCATAGTGA | −15_6-5_internal_loop-asymmetric_AGUGGA-AGUGA, | −15 | 33 |
| | | | GCCAGGAGGAAAAAGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 23_2-2_bulge- | | |
| | | | CTGGGCTGGA | symmetric_GG-AA, 33_6-6_internal_loop- | | |
| | | | | symmetric_GAGUGA-AGUGAG | | |
| 42 | 0.92.62(−15, +33)+4(1/1) | 2832 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCACTCTGCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_12_1- | | |
| | | | CTGGGCTGGA | 1_mismatch_G-G, 33_6-6_internal_loop- | | |
| | | | | symmetric_GAGUGA-AGUGAG | | |
| 42 | 0.92.62(−15, +33)+5(1/1) | 2833 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCACTCACCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_13_1- | | |
| | | | CTGGGCTGGA | 1_mismatch_A-A, 33_6-6_internal_loop- | | |
| | | | | symmetric_GAGUGA-AGUGAG | | |
| 42 | 0.92.62(−15, +33)+6(1/1) | 2834 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCACTGTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_14_1- | | |
| | | | CTGGGCTGGA | 1_mismatch_G-G, 33_6-6_internal_loop- | | |
| | | | | symmetric_GAGUGA-AGUGAG | | |
| 42 | 0.92.62(−15, +33)+7(1/1) | 2835 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCACACTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_15_1- | | |
| | | | CTGGGCTGGA | 1_mismatch_A-A, 33_6-6_internal_loop- | | |
| | | | | symmetric_GAGUGA-AGUGAG | | |
| 42 | 0.92.62(−15, +33)+8(1/1) | 2836 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGCAGTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_16_1- | | |
| | | | CTGGGCTGGA | 1_mismatch_G-G, 33_6-6_internal_loop- | | |
| | | | | symmetric_GAGUGA-AGUGAG | | |
| 42 | 0.92.62(−15, +33)+9(1/1) | 2837 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGTTCTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_17_1- | | |
| | | | CTGGGCTGGA | 1_mismatch_U-U,_18_1-1_wobble_G-U, 33_6- | | |
| | | | | 6_internal_loop-symmetric_GAGUGA-AGUGAG | | |
| 42 | 0.92.62(−15, +33)+10(1/1) | 2838 | TGTGGTTGTTTTGCCGGCACCATAGTGA | −15_6-5_internal_loop-asymmetric_AGUGGA-AGUGA, | −15 | 33 |
| | | | GCCAGGAGGCCAAAGAACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_18_1- | | |
| | | | CTGGGCTGGA | 1_mismatch_G-A, 33_6-6_internal_loop- | | |
| | | | | symmetric_GAGUGA-AGUGAG | | |
| 42 | 0.92.62(−15, +33)+11(1/1) | 2839 | TGTGGTTGTTTTGCCGGCACCATAGTGA | −15_6-5_internal_loop-asymmetric_AGUGGA-AGUGA, | −15 | 33 |
| | | | GCCAGGAGGCCAAACCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U,_19_1- | | |
| | | | CTGGGCTGGA | 1_mismatch_C-C, 33_6-6_internal_loop- | | |
| | | | | symmetric_GAGUGA-AGUGAG | | |
| 42 | 0.92.62(−15, +33)+12(1/1) | 2840 | TGTGGTTGTTTTGCCGGCACCATAGTGA | −15_6-5_internal_loop-asymmetric_AGUGGA-AGUGA, | −15 | 33 |
| | | | GCCAGGAGGCCAATGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 20_1- | | |
| | | | CTGGGCTGGA | 1_mismatch_U-U, 33_6-6_internal_loop- | | |
| | | | | symmetric_GAGUGA-AGUGAG | | |
| 42 | 0.92.62(−15, +33)+13(1/1) | 2841 | TGTGGTTGTTTTGCCGGCACCATAGTGA | −15_6-5_internal_loop-asymmetric_AGUGGA-AGUGA, | −15 | 33 |
| | | | GCCAGGAGGCCATAGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 21_1- | | |
| | | | CTGGGCTGGA | 1_mismatch_U-U, 33_6-6_internal_loop- | | |
| | | | | symmetric_GAGUGA-AGUGAG | | |
| 42 | 0.92.62(−15, +33)+14(1/1) | 2842 | TGTGGTTGTTTTGCCGGCACCATAGTGA | −15_6-5_internal_loop-asymmetric_AGUGGA-AGUGA, | −15 | 33 |
| | | | GCCAGGAGGTCTAAGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 22_1- | | |
| | | | CTGGGCTGGA | 1_mismatch_U-U, 24_1-1_wobble_G-U, 33_6- | | |
| | | | | 6_internal_loop-symmetric_GAGUGA-AGUGAG | | |
| 42 | 0.92.62(−15, +33)+15(1/1) | 2843 | TGTGGTTGTTTTGCCGGCACCATAGTGA | −15_6-5_internal_loop-asymmetric_AGUGGA-AGUGA, | −15 | 33 |
| | | | GCCAGGAGGCGAAAGCACTCTCCAGTG | −1->0_2-2_bulge-symmetric_GA-CG, 6_1- | | |
| | | | AGAACTCGGACACACAGCCTGAGTGAG | 1_mismatch_G-A, 8_1-1_mismatch_C-U, 23_1- | | |
| | | | CTGGGCTGGA | 1_mismatch_G-G, 33_6-6_internal_loop- | | |
| | | | | symmetric_GAGUGA-AGUGAG | | |

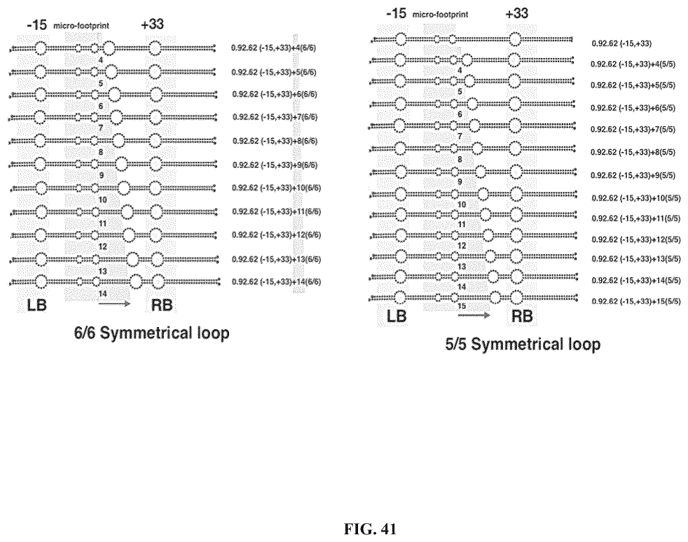

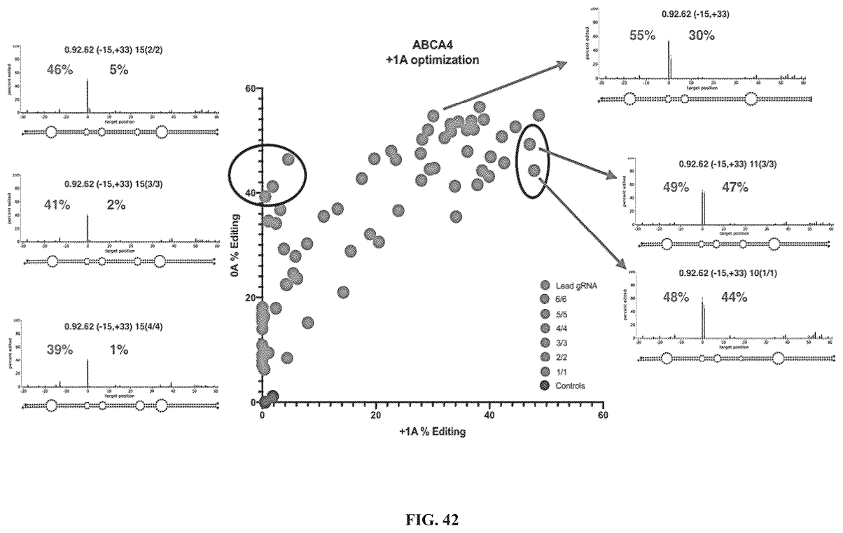

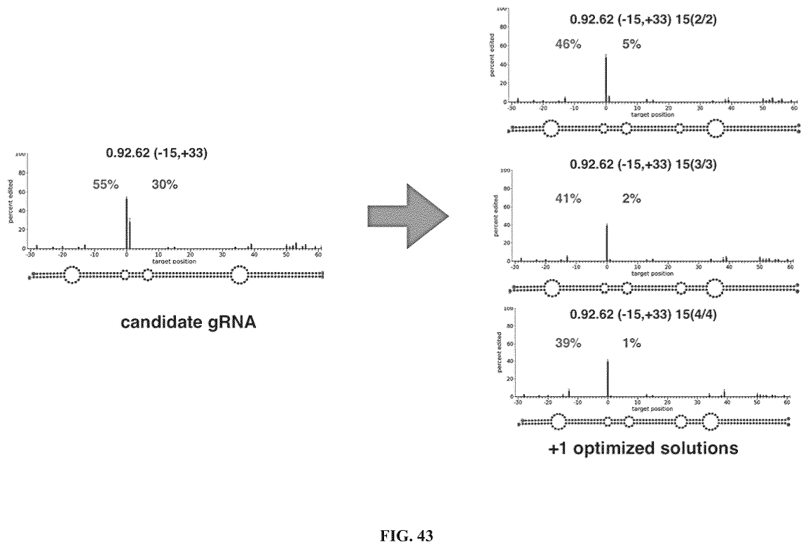

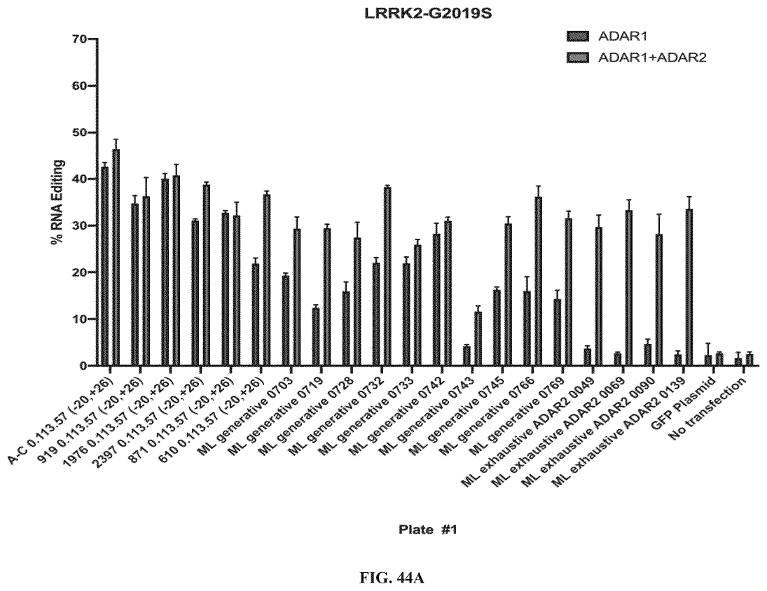

Symmetrical internal loops and symmetrical bulges (6/6, 5/5, 4/4,3/3, 2/2, 1/1) were inserted and tiled throughout the engineered guide RNA at single nucleotide resolution (scanning positions: 4 to 15) and the effect of each on percent editing was assessed in HEK293 cells. Most of the structural features located at position 15 eliminated +1 editing, especially the 4/4, 3/3 and 2/2 structural features, without severely affecting the on-target RNA editing efficiency. FIG. 41 depicts tiling of 6/6 symmetrical loops and 5/5 symmetrical internal loops in the guide-target ABCA4 RNA scaffold. As shown in FIG. 42, engineered guide RNAs were plotted based on their ability to facilitate on target adenosine editing against +1 editing efficiency. As shown in FIG. 42, engineered guide RNAs 0.92.62 (−15,+33) 15(2/2), 0.92.62 (−15,+33) 15(3/3), and 0.92.62 (−15,+33) 15(4/4), each identical with respect to micro-footprint and macro-footprint other than the size of the structural feature inserted at position 15 of the micro-footprint, displayed significant on-target adenosine editing while having less than 5% +1 editing.

Thus, this example demonstrates that local perturbation of the micro-footprint through insertion of bulges or internal loops can be used to minimize off target +1 editing in a candidate ABCA4 engineered guide RNA.

### Example 21

### In-Cell Editing Efficiencies for Machine Learning Derived LRRK2 Engineered Guide RNAs (−20, +26)

This example describes targeting the LRRK2 G2019S mutation for editing in vitro, in cells, using engineered guide RNAs of the present disclosure that were derived using multiple machine learning (ML) models, including an exhaustive ML model and a generative ML model. 750 ng of plasmid was transfected in cells (20,000 cells/well) of 68 cell line (LRRK2 cDNA minigene+ADAR1) and 219 cell line (LRRK2 cDNA minigene+ADAR1+2). 4 technical replicates were performed for each engineered guide RNA. Each engineered guide RNA tested contained a barbell macro-footprint of symmetrical internal loops with coordinates at −20 and +26 relative to the target A.

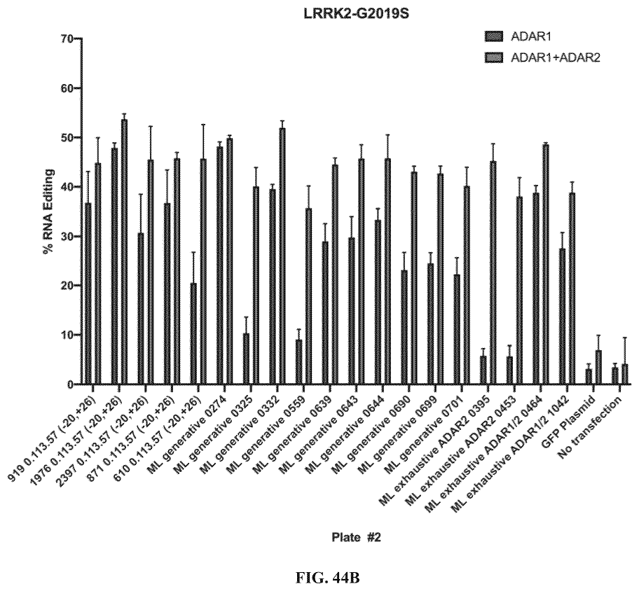

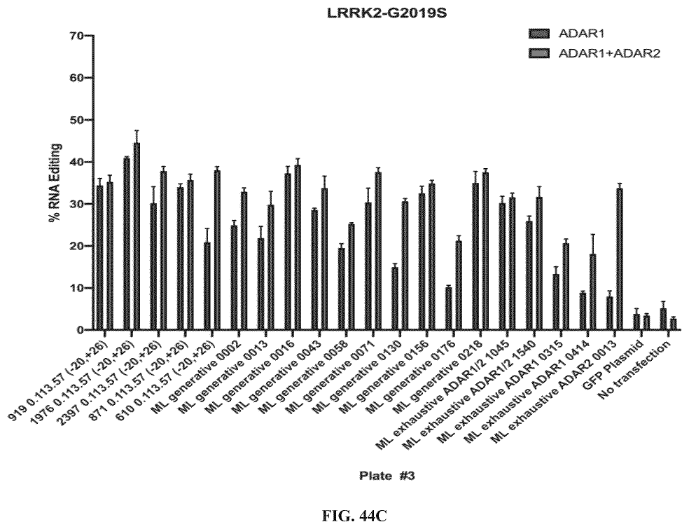

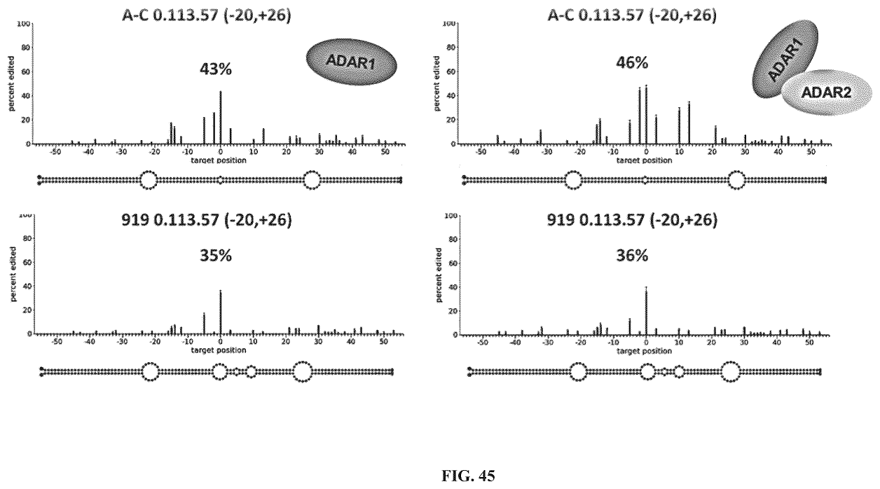

FIG. 44A-FIG. 44C provide a summary of the RNA editing efficiency of each LRRK2 engineered guide RNA tested via ADAR. The following LRRK2 engineered guide RNAs recited in TABLE 9 are utilized in the summary of RNA editing provided in FIG. 44A-FIG. 44C. Each engineered guide RNA sequence can also be represented as a DNA sequence in which each U is replaced with a T.

TABLE 9
LRRK2 ML Engineered Guide RNA Sequences

| SEQ | | Engineered | | Left | Right |
|---|---|---|---|---|---|
| ID | Guide | Guide RNA | | Barbell | Barbell |
| NO | Name | Sequence | Structural Features(target/guide) | Position | Position |
| 2686 | Ml_ | CCCUGGUGUG | −49_1-1_mismatch_C-A | −20 | 26 |
| | Exhaustive_ | CCCUCUGAUG | −20_6-6_internal_loop-symmetric_CAUCAU-UACUAC | | |
| | ADAR1_ | UUUUUUAGGG | −13_1-1_mismatch_G-A | | |
| | 0315 | GAUUCUACAG | −3_1-1_wobble_U-G | | |
| | | CAGGACUGAG | 1_1-1_wobble_G-U | | |
| | | CAAUCUUGUG | 2_1-1_mismatch_C-C | | |
| | | GUCAGCAAUA | 13_1-1_mismatch_A-G | | |
| | | UUUGCAUACU | 26_6-6_internal_loop-symmetric_GGGGAU-UAGGGG | | |
| | | ACGCAGCAUU | 34_1-1_wobble_G-U | | |
| | | GGGAUACAGU | | | |
| | | GUGAAAAGCA | | | |
| | | GCA | | | |
| 2687 | Ml_ | CCCUGGUGUG | −49_1-1_mismatch_C-A | −20 | 26 |
| | Exhaustive_ | CCCUCUGAUG | −20_6-6_internal_loop-symmetric_CAUCAU-UACUAC | | |
| | ADAR1_ | UUUUUUAGGG | −13_1-1_mismatch_G-A | | |
| | 0414 | GAUUCUACAG | −3_1-1_wobble_U-G | | |
| | | CAGGACUGAG | 0->1 2-2_bulge-symmetric_AG-GA | | |
| | | CAAUGGAGUG | 13_1-1_mismatch_A-G | | |
| | | GUCAGCAAUA | 26_6-6_internal_loop-symmetric_GGGGAU-UAGGGG | | |
| | | UUUGCAUACU | 34_1-1_wobble_G-U | | |
| | | ACGCAGCAUU | | | |
| | | GGGAUACAGU | | | |
| | | GUGAAAAGCA | | | |
| | | GCA | | | |
| 2688 | ML_ | CCCUGGUGUG | −49_1-1_mismatch_C-A | −20 | 26 |
| | Exhaustive_ | CCCUCUGAUG | −20_6-6_internal_loop-symmetric_CAUCAU-UACUAC | | |
| | ADAR2_ | UUUUUUAGGG | −10_1-1_mismatch_U-C | | |
| | 0313 | GAUUCUACAG | −1->0_2-2_bulge-symmetric_CA-GU | | |
| | | CACGACUGAG | 4_1-1_wobble_U-G | | |
| | | CAGUGCGUUA | 13_2-2_bulge-symmetric_AC-CG | | |
| | | GUCAGCCAUC | 26_6-6_internal_loop-symmetric_GGGGAU-UAGGGG | | |
| | | UUUGCAUACU | 34_1-1_wobble_G-U | | |
| | | ACGCAGCAUU | | | |
| | | GGGAUACAGU | | | |
| | | GUGAAAAGCA | | | |
| | | GCA | | | |
| 2689 | Ml_ | CCCUGGUGUG | −49_1-1_mismatch_C-A | −20 | 26 |
| | Exhaustive_ | CCCUCUGAUG | −20_6-6_internal_loop-symmetric_CAUCAU-UACUAC | | |
| | ADAR2_ | UUUUUUAGGG | −11_1-1_mismatch_U-C | | |
| | 0049 | GAUUCUACAG | −1->0_2-2_bulge-symmetric_CA-GU | | |
| | | CAUUACUCAG | 4_1-1_wobble_U-G | | |
| | | CAGUGCGUUA | 9_1-1_mismatch_C-C | | |
| | | GUCAGCACUC | 14_1-1_mismatch_C-U | | |
| | | UUUGCAUACU | 26_6-6_internal_loop-symmetric_GGGGAU-UAGGGG | | |
| | | ACGCAGCAUU | 34_1-1_wobble_G-U | | |
| | | GGGAUACAGU | | | |
| | | GUGAAAAGCA | | | |
| | | GCA | | | |
| 2690 | Ml_ | CCCUGGUGUG | −49_1-1_mismatch_C-A | −20 | 26 |
| | Exhaustive_ | CCCUCUGAUG | −20_6-6_internal_loop-symmetric_CAUCAU-UACUAC | | |
| | ADAR2_ | UUUUUUAGGG | −7_1-1_wobble U-G | | |
| | 0069 | GAUUCUACAG | −1->0_2-2_bulge-symmetric_CA-GU | | |
| | | CACGACUGAG | 4_1-1_wobble U-G | | |
| | | CAGUGCGCUA | 13_2-2_bulge-symmetric_AC-CG | | |
| | | GUCGGCAAUC | 26_6-6_internal_loop-symmetric_GGGGAU-UAGGGG | | |
| | | UUUGCAUACU | 34_1-1 wobble_G-U | | |
| | | ACGCAGCAUU | | | |
| | | GGGAUACAGU | | | |
| | | GUGAAAAGCA | | | |
| | | GCA | | | |
| 2691 | ML_ | CCCUGGUGUG | −49_1-1_mismatch_C-A | −20 | 26 |
| | Exhaustive_ | CCCUCUGAUG | −20_6-6_internal_loop-symmetric_CAUCAU-UACUAC | | |
| | ADAR2_ | UUUUUUAGGG | −2->0_3-3_bulge-symmetric_ACA-GUG | | |
| | 0090 | GAUUCUACAG | 4_1-1_wobble_U-G | | |
| | | CAAUACUCAG | 9_1-1_mismatch_C-C | | |
| | | CAGUGCGUGA | 14_1-1_mismatch_C-A | | |
| | | GUCAGCAAUC | 26_6-6_internal_loop-symmetric_GGGGAU-UAGGGG | | |
| | | UUUGCAUACU | 34_1-1_wobble_G-U | | |
| | | ACGCAGCAUU | | | |
| | | GGGAUACAGU | | | |
| | | GUGAAAAGCA | | | |
| | | GCA | | | |
| 2692 | Ml_ | CCCUGGUGUG | −49_1-1_mismatch_C-A | −20 | 26 |
| | Exhaustive_ | CCCUCUGAUG | −20_6-6_internal_loop-symmetric_CAUCAU-UACUAC | | |
| | ADAR2_ | UUUUUUAGGG | −7_1-1_wobble_U-G | | |
| | 0139 | GAUUCUACAG | −1->0_2-2_bulge-symmetric_CA-GC | | |
| | | CACGACUGAG | 4_1-1 wobble_U-G | | |
| | | CAGUGCGCUA | 13_2-2_bulge-symmetric_AC-CG | | |
| | | GUCGGCAAUC | 26_6-6_internal_loop-symmetric_GGGGAU-UAGGGG | | |
| | | UUUGCAUACU | 34_1-1_wobble_G-U | | |
| | | ACGCAGCAUU | | | |
| | | GGGAUACAGU | | | |
| | | GUGAAAAGCA | | | |
| | | GCA | | | |
| 2693 | Ml_ | CCCUGGUGUG | −49_1-1_mismatch_C-A | −20 | 26 |
| | Exhaustive_ | CCCUCUGAUG | −20_6-6_internal_loop-symmetric_CAUCAU-UACUAC | | |
| | ADAR2_ | UUUUUUAGGG | −13_1-1_mismatch_G-A | | |
| | 0395 | GAUUCUACAG | −1->0_2-2_bulge-symmetric_CA-GC | | |
| | | CACGACUGAG | 4_1-1_wobble_U-G | | |
| | | CAGUGCGCUA | 13_2-2_bulge-symmetric_AC-CG | | |
| | | GUCAGCAAUA | 26_6-6_internal_loop-symmetric_GGGGAU-UAGGGG | | |
| | | UUUGCAUACU | 34_1-1_wobble_G-U | | |
| | | ACGCAGCAUU | | | |
| | | GGGAUACAGU | | | |
| | | GUGAAAAGCA | | | |
| | | GCA | | | |
| 2694 | Ml_ | CCCUGGUGUG | −49_1-1_mismatch_C-A | −20 | 26 |
| | Exhaustive_ | CCCUCUGAUG | −20_6-6_internal_loop-symmetric_CAUCAU-UACUAC | | |
| | ADAR2_ | UUUUUUAGGG | −10_1-1_mismatch_U-C | | |
| | 0453 | GAUUCUACAG | −1->0_2-2_bulge-symmetric_CA-GC | | |
| | | CAAUACUCAG | 4_1-1_wobble_U-G | | |
| | | CAGUGCGCUA | 9_1-1_mismatch_C-C | | |
| | | GUCAGCCAUC | 14_1-1_mismatch_C-A | | |
| | | UUUGCAUACU | 26_6-6_internal_loop-symmetric_GGGGAU-UAGGGG | | |
| | | ACGCAGCAUU | 34_1-1_wobble_G-U | | |
| | | GGGAUACAGU | | | |
| | | GUGAAAAGCA | | | |
| | | GCA | | | |
| 2695 | Ml_ | CCCUGGUGUG | −49_1-1_mismatch_C-A | −20 | 26 |
| | Exhaustive_ | CCCUCUGAUG | −20_6-6_internal_loop-symmetric_CAUCAU-UACUAC | | |
| | ADAR12_ | UUUUUUAGGG | −7_1-1_wobble_U-G | | |
| | 0464 | GAUUCUACAG | 0_1-1_mismatch_A-C | | |
| | | CAUGACUGAG | 2_1-1_mismatch_C-C | | |
| | | CAGUCCCGUA | 4_1-1_wobble_U-G | | |
| | | GUCGGCAAUC | 13_2-2_bulge-symmetric_AC-UG | | |
| | | UUUGCAUACU | 26_6-6_internal_loop-symmetric_GGGGAU-UAGGGG | | |
| | | ACGCAGCAUU | 34_1-1_wobble_G-U | | |
| | | GGGAUACAGU | | | |
| | | GUGAAAAGCA | | | |
| | | GCA | | | |
| 2696 | Ml_ | CCCUGGUGUG | −49_1-1_mismatch_C-A | −20 | 26 |
| | Exhaustive_ | CCCUCUGAUG | −20_6-6_internal_loop-symmetric_CAUCAU-UACUAC | | |
| | ADAR12_ | UUUUUUAGGG | −13_1-1_mismatch_G-A | | |
| | 1042 | GAUUCUACAG | 0_1-1_mismatch_A-C | | |
| | | CAAUACUCAG | 2_1-1_mismatch_C-U | | |
| | | CAGUUCCGUA | 4_1-1_wobble U-G | | |
| | | GUCAGCAAUA | 9_1-1_mismatch_C-C | | |
| | | UUUGCAUACU | 14_1-1_mismatch_C-A | | |
| | | ACGCAGCAUU | 26_6-6_internal_loop-symmetric_GGGGAU-UAGGGG | | |
| | | GGGAUACAGU | 34_1-1_wobble_G-U | | |
| | | GUGAAAAGCA | | | |
| | | GCA | | | |
| 2697 | ML_ | CCCUGGUGUG | −49_1-1_mismatch_C-A | −20 | 26 |
| | Exhaustive_ | CCCUCUGAUG | −20_6-6_internal_loop-symmetric_CAUCAU-UACUAC | | |
| | ADAR12_ | UUUUUUAGGG | −13_1-1_mismatch_G-A | | |
| | 104 | GAUUCUACAG | 0_1-1_mismatch_A-C | | |
| | | CAUUACUCAG | 2_1-1_mismatch_C-C | | |
| | | CAGUCCCGUA | 4_1-1_wobble_U-G | | |
| | | GUCAGCAAUA | 9_1-1_mismatch_C-C | | |
| | | UUUGCAUACU | 14_1-1_mismatch_C-U | | |
| | | ACGCAGCAUU | 26_6-6_internal_loop-symmetric_GGGGAU-UAGGGG | | |
| | | GGGAUACAGU | 34_1-1_wobble_G-U | | |
| | | GUGAAAAGCA | | | |
| | | GCA | | | |
| 2698 | Ml_ | CCCUGGUGUG | −49_1-1_mismatch_C-A | −20 | 26 |
| | Exhaustive_ | CCCUCUGAUG | −20 6-6_internal_loop-symmetric_CAUCAU-UACUAC | | |
| | ADAR12_ | UUUUUUAGGG | −9_1-1_mismatch_G-A | | |
| | 1540 | GAUUCUACAG | 0_1-1_mismatch_A-C | | |
| | | CAUUACUCAG | 2_2-2_bulge-symmetric_CA-AU | | |
| | | CAAAUCCGUA | 9_1-1_mismatch_C-C | | |
| | | GUCAGAAAUC | 14_1-1_mismatch_C-U | | |
| | | UUUGCAUACU | 26_6-6_internal_loop-symmetric_GGGGAU-UAGGGG | | |
| | | ACGCAGCAUU | 34_1-1_wobble_G-U | | |
| | | GGGAUACAGU | | | |
| | | GUGAAAAGCA | | | |
| | | GCA | | | |
| 2699 | Ml_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0002 | UUUUUUAGGG | −10_1-1_mismatch_U-C | | |
| | | GAUUCUAGAG | −7_1-1_wobble_U-G | | |
| | | AGGUACUGUG | −3_1-1_wobble_U-G | | |
| | | CCAUCCCGUG | 0_1-1_mismatch_A-C | | |
| | | GUCGGCCAUC | 2_1-1_mismatch_C-C | | |
| | | UUUGCAUACU | 5_1-1_mismatch_U-C | | |
| | | ACGCAGCAUU | 8_1-1_mismatch_U-U | | |
| | | GGGAUACAGU | 15_1-1_wobble_U-G | | |
| | | GUGAAAAGCA | 16_1-1_mismatch_G-A | | |
| | | GCA | 19_1-1_mismatch_G-G | | |
| | | | 26_6-6_internal_loop-symmetric_GGGGAU-UAGGGG | | |
| | | | 34_1-1_wobble_G-U | | |
| 2700 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0013 | UUUUUUAGGG | −7_1-1_wobble_U-G | | |
| | | GAUUCUAGAG | −3_1-1_wobble_U-G | | |
| | | CAGGACGGUG | 0_1-1_mismatch_A-C | | |
| | | CAGUCCCGUG | 2_1-1_mismatch_C-C | | |
| | | GUCGGCAAUC | 4_1-1_wobble_U-G | | |
| | | UUUGCAUACU | 8_1-1_mismatch_U-U | | |
| | | ACGCAGCAUU | 10_1-1_mismatch_A-G | | |
| | | GGGAUACAGU | 13_1-1_mismatch_A-G | | |
| | | GUGAAAAGCA | 19_1-1_mismatch_G-G | | |
| | | GCA | 26_6-6_internal_loop-symmetric_GGGGAU-UAGGGG | | |
| | | | 34_1-1_wobble_G-U | | |
| 2701 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0016 | UUUUUUAGGG | −10_2-2_bulge-symmetric_UU-CU | | |
| | | GAUUCUACAG | −8_1-1_mismatch_C-C | | |
| | | GAGGACUGGG | −7_1-1_wobble_U-G | | |
| | | CAGUCCCGUG | −3_1-1_wobble_U-G | | |
| | | GUCGCCCUUC | 0_1-1_mismatch_A-C | | |
| | | UUUGCAUACU | 2_1-1_mismatch_C-C | | |
| | | ACGCAGCAUU | 4_1-1_wobble U-G | | |
| | | GGGAUACAGU | 8_1-1_wobble U-G | | |
| | | GUGAAAAGCA | 13_1-1_mismatch_A-G | | |
| | | GCA | 16_1-1_mismatch_G-G | | |
| | | | 26_6-6_internal_loop-symmetric_GGGGAU-UAGGGG | | |
| | | | 34_1-1_wobble_G-U | | |
| 2702 | Ml_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0043 | UUUUUUAGGG | −10_1-1_mismatch_U-C | | |
| | | GAUUCUACAG | −9_1-1_wobble_G-U | | |
| | | CGGUACUGUG | −7_1-1_wobble U-G | | |
| | | CAAAUCCGUG | −3_1-1_wobble_U-G | | |
| | | GUCGGUCAUC | 0_1-1_mismatch_A-C | | |
| | | UUUGCAUACU | 2_2-2_bulge-symmetric_CA-AU | | |
| | | ACGCAGCAUU | 8_1-1_mismatch_U-U | | |
| | | GGGAUACAGU | 15_1-1_wobble_U-G | | |
| | | GUGAAAAGCA | 26_6-6_internal_loop-symmetric_GGGGAU-UAGGGG | | |
| | | GCA | 34_1-1_wobble_G-U | | |
| 2703 | Ml_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0058 | UUUUUUAGGG | −10_1-1_mismatch_U-C | | |
| | | GAUUCUACGG | −7_1-1_wobble_U-G | | |
| | | CAGGACUGGG | −3_1-1_wobble U-G | | |
| | | GAAAUCCGUG | 0_1-1_mismatch_A-C | | |
| | | GUCGGCCAUC | 2_2-2_bulge-symmetric_CA-AU | | |
| | | UUUGCAUACU | 6_1-1_mismatch_G-G | | |
| | | ACGCAGCAUU | 8_1-1_wobble_U-G | | |
| | | GGGAUACAGU | 13_1-1_mismatch_A-G | | |
| | | GUGAAAAGCA | 18_1-1_wobble_U-G | | |
| | | GCA | 26_6-6_internal_loop-symmetric_GGGGAU-UAGGGG | | |
| | | | 34_1-1_wobble_G-U | | |
| 2704 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0071 | UUUUUUAGGG | −10_1-1_mismatch_U-C | | |
| | | GAUUCUACAG | −8_1-1_mismatch_C-U | | |
| | | CGGUACUGCG | −7_1-1_wobble_U-G | | |
| | | CAGUCCCGUG | −6_1-1_mismatch_G-G | | |
| | | GUGGUCCAUC | −3_1 _wobble_U-G | | |
| | | UUUGCAUACU | 0_1-1_mismatch_A-C | | |
| | | ACGCAGCAUU | 2_1-1_mismatch_C-C | | |
| | | GGGAUACAGU | 4_1-1_wobble_U-G | | |
| | | GUGAAAAGCA | 8_1-1_mismatch_U-C | | |
| | | GCA | 15_1-1_wobble_U-G | | |
| | | | 26_6-6_internal_loop-symmetric_GGGGAU-UAGGGG | | |
| | | | 34_1-1_wobble_G-U | | |
| 2705 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0130 | UUUUUUAGGG | −7_1-1_wobble_U-G | | |
| | | GAUUCUAUAG | −3_1-1_wobble_U-G | | |
| | | CAGGAAUGAG | 0_1-1_mismatch_A-C | | |
| | | GAAAUCCGUG | 2_2-2_bulge-symmetric_CA-AU | | |
| | | GUCGGCAAUC | 6_1-1_mismatch_G-G | | |
| | | UUUGCAUACU | 11_3-3_bulge-symmetric_GUA-GAA | | |
| | | ACGCAGCAUU | 19_1-1_wobble_G-U | | |
| | | GGGAUACAGU | 26_6-6_internal_loop-symmetric_GGGGAU-UAGGGG | | |
| | | GUGAAAAGCA | 34_1-1 wobble_G-U | | |
| | | GCA | | | |
| 2706 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0156 | UUUUUUAGGG | −9_2-2_bulge-symmetric_UG-AU | | |
| | | GAUUCUACAG | −7_1-1_wobble_U-G | | |
| | | CAGUACUCAG | −3_1-1_wobble_U-G | | |
| | | GAAAUCCGUG | 0_1-1_mismatch_A-C | | |
| | | GUCGGAUAUC | 2_2-2_bulge-symmetric_CA-AU | | |
| | | UUUGCAUACU | 6_1-1_mismatch_G-G | | |
| | | ACGCAGCAUU | 9_1-1_mismatch_C-C | | |
| | | GGGAUACAGU | 26_6-6_internal_loop-symmetric_GGGGAU-UAGGGG | | |
| | | GUGAAAAGCA | 34_1-1_wobble G-U | | |
| | | GCA | | | |
| 2707 | Ml_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0176 | UUUUUUAGGG | −7_1-1_wobble_U-G | | |
| | | GAUUCUAAAG | −3_1-1_wobble_U-G | | |
| | | CAGGAGUGAG | 0->3_4-4_bulge-symmetric_AGCA-AUCC | | |
| | | CAGAUCCGUG | 4_1-1_wobble_U-G | | |
| | | GUCGGCAAUC | 11_1-1_mismatch_G-G | | |
| | | UUUGCAUACU | 13_1-1_mismatch_A-G | | |
| | | ACGCAGCAUU | 19_1-1_mismatch_G-A | | |
| | | GGGAUACAGU | 26_6-6_internal_loop-symmetric_GGGGAU-UAGGGG | | |
| | | GUGAAAAGCA | 34_1-1_wobble_G-U | | |
| | | GCA | | | |
| 2708 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0218 | UUUUUUAGGG | −8_2-2_bulge-symmetric_GC-UA | | |
| | | GAUUCUACAG | −3_1-1_wobble_U-G | | |
| | | GUGUACUGUG | 0_1-1_mismatch_A-C | | |
| | | AAAUCCCGUG | 2_1-1_mismatch_C-C | | |
| | | GUCAUAAAUC | 6_1-1_mismatch_G-A | | |
| | | UUUGCAUACU | 8_1-1_mismatch_U-U | | |
| | | ACGCAGCAUU | 15_2-2_bulge-symmetric_UG-GU | | |
| | | GGGAUACAGU | 26_6-6_internal_loop-symmetric_GGGGAU-UAGGGG | | |
| | | GUGAAAAGCA | 34_1-1_wobble_G-U | | |
| | | GCA | | | |
| 2709 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0274 | UUUUUUAGGG | −8 2-2_bulge-symmetric_GC-UA | | |
| | | GAUUCUACAG | −7_1-1_wobble U-G | | |
| | | CAGUACUGUC | −3_1-1_wobble_U-G | | |
| | | CAGUCCCGUG | 0_1-1_mismatch_A-C | | |
| | | GUCGUAAAUC | 2_1-1_mismatch_C-C | | |
| | | UUUGCAUACU | 4_1-1_wobble_U-G | | |
| | | ACGCAGCAUU | 7 2-2_bulge-symmetric_CU-UC | | |
| | | GGGAUACAGU | 26_6-6_internal_loop-symmetric_GGGGAU-UAGGGG | | |
| | | GUGAAAAGCA | 34_1-1_wobble_G-U | | |
| | | GCA | | | |
| 2710 | ML_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0325 | UUUUUUAGGG | −10_1-1_mismatch_U-U | | |
| | | GAUUCUACAU | −7_1-1_wobble U-G | | |
| | | CAGGACUGAG | −3_1-1_wobble_U-G | | |
| | | CUUUUCCGAG | −2->5_8-8_internal_loop-symmetric_ACAGCAUU- | | |
| | | GUCGGCUAUC | UUUUCCGA | | |
| | | UUUGCAUACU | 13_1-1_mismatch_A-G | | |
| | | ACGCAGCAUU | 17_1-1_mismatch_C-U | | |
| | | GGGAUACAGU | 26_6-6_internal_loop-symmetric_GGGGAU-UAGGGG | | |
| | | GUGAAAAGCA | 34_1-1_wobble G-U | | |
| | | GCA | | | |
| 2711 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0332 | UUUUUUAGGG | −7_1-1_wobble_U-G | | |
| | | GAUUCUACAU | 0_1-1_mismatch_A-C | | |
| | | CAGUACUGGG | 2_1-1_mismatch_C-U | | |
| | | CAGUUCCGUA | 4_1-1_wobble U-G | | |
| | | GUCGGCAAUC | 8_1-1_wobble_U-G | | |
| | | UUUGCAUACU | 17_1-1_mismatch_C-U | | |
| | | ACGCAGCAUU | 26_6-6_internal_loop-symmetric_GGGGAU-UAGGGG | | |
| | | GGGAUACAGU | 34_1-1 wobble_G-U | | |
| | | GUGAAAAGCA | | | |
| | | GCA | | | |
| 2712 | Ml_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0559 | UUUUUUAGGG | −11_1-1_mismatch_U-C | | |
| | | GAUUCUACAG | −7_1-1_wobble_U-G | | |
| | | CAGUACUGGG | −1->0_2-2_bulge-symmetric_CA-GC | | |
| | | CAGUGCGCUA | 4_1-1_wobble_U-G | | |
| | | GUCGGCACUC | 8_1-1_wobble U-G | | |
| | | UUUGCAUACU | 26_6-6_internal_loop-symmetric_GGGGAU-UAGGGG | | |
| | | ACGCAGCAUU | 34_1-1_wobble G-U | | |
| | | GGGAUACAGU | | | |
| | | GUGAAAAGCA | | | |
| | | GCA | | | |
| 2713 | ML_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0639 | UUUUUUAGGG | −6_1-1_mismatch_G-A | | |
| | | GAUUCUACAG | −3->2 6-6_internal_loop-symmetric_UACAGC- | | |
| | | CAAUACUCAC | CCCGAU | | |
| | | CAGUCCCGAU | 4_1-1_wobble U-G | | |
| | | GUAAGCAAUC | 7_3-3_bulge-symmetric_CUC-CAC | | |
| | | UUUGCAUACU | 14_1-1_mismatch_C-A | | |
| | | ACGCAGCAUU | 26_6-6_internal_loop-symmetric_GGGGAU-UAGGGG | | |
| | | GGGAUACAGU | 34_1-1_wobble_G-U | | |
| | | GUGAAAAGCA | | | |
| | | GCA | | | |
| 2714 | Ml_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0643 | UUUUUUAGGG | −7_1-1_wobble_U-G | | |
| | | GAUUCUACGG | −3->6_10-10_internal_loop- | | |
| | | CAGUACUGUG | symmetric_UACAGCAUUG-AAAUCCCGAU | | |
| | | AAAUCCCGAU | 8_1-1_mismatch_U-U | | |
| | | GUCGGCAAUC | 18_1-1 wobble_U-G | | |
| | | UUUGCAUACU | 26_6-6_internal_loop-symmetric_GGGGAU-UAGGGG | | |
| | | ACGCAGCAUU | 34_1-1 wobble_G-U | | |
| | | GGGAUACAGU | | | |
| | | GUGAAAAGCA | | | |
| | | GCA | | | |
| 2715 | Ml_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0644 | UUUUUUAGGG | −7_0-1_bulge-asymmetric_-G | | |
| | | GAUUCUACCG | −6_1-1_wobble_G-U | | |
| | | CAGGACGGAG | −2->5_8-7_internal_loop-asymmetric_ACAGCAUU- | | |
| | | CUAUCCCGAG | UAUCCCG | | |
| | | UUAGGCAAUC | 10_1-1_mismatch_A-G | | |
| | | UUUGCAUACU | 13_1-1_mismatch_A-G | | |
| | | ACGCAGCAUU | 18_1-1_mismatch_U-C | | |
| | | GGGAUACAGU | 26_6-6_internal_loop-symmetric_GGGGAU-UAGGGG | | |
| | | GUGAAAAGCA | 34_1-1 wobble_G-U | | |
| | | GCA | | | |
| 2716 | Ml_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0690 | UUUUUUAGGG | −14_2-2_bulge-symmetric_AA-GA | | |
| | | GAUUCUACGG | −9_4-4_bulge-symmetric_AUUG-ACCC | | |
| | | CAUGACUGAU | −7_1-1_wobble_U-G | | |
| | | CAGUCCCGUG | −3_1-1_wobble_U-G | | |
| | | GUCGGACCCC | 0_1-1_mismatch_A-C | | |
| | | GAUGCAUACU | 2_1-1_mismatch_C-C | | |
| | | ACGCAGCAUU | 4_1-1_wobble_U-G | | |
| | | GGGAUACAGU | 7_1-1_mismatch_C-U | | |
| | | GUGAAAAGCA | 13_2-2_bulge-symmetric_AC-UG | | |
| | | GCA | 18_1-1_wobble_U-G | | |
| | | | 26_6-6_internal_loop-symmetric_GGGGAU-UAGGGG | | |
| | | | 34_1-1_wobble_G-U | | |
| 2717 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0699 | UUUUUUAGGG | −9 7-7_internal_loop-symmetric_AAGAUUG- | | |
| | | GAUUCUACGG | ACCCCCG | | |
| | | CAAGACUGGG | −7_1-1_wobble_U-G | | |
| | | CAGUCCCGUG | −3_1-1_wobble_U-G | | |
| | | GUCGGACCCC | 0_1-1_mismatch_A-C | | |
| | | CGUGCAUACU | 2_1-1_mismatch_C-C | | |
| | | ACGCAGCAUU | 4_1-1_wobble_U-G | | |
| | | GGGAUACAGU | 8_1-1_wobble_U-G | | |
| | | GUGAAAAGCA | 13_2-2_bulge-symmetric_AC-AG | | |
| | | GCA | 18_1-1_wobble_U-G | | |
| | | | 26_6-6_internal_loop-symmetric_GGGGAU-UAGGGG | | |
| | | | 34_1-1_wobble_G-U | | |
| 2718 | Ml_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0701 | UUUUUUAGGG | −14_2-2_bulge-symmetric_AA-CG | | |
| | | GAUUCUACGG | −9_3-3_bulge-symmetric_UUG-ACC | | |
| | | CAGGACUGGG | −7_1-1_wobble_U-G | | |
| | | CUUUCCCGUG | −3_1-1_wobble_U-G | | |
| | | GUCGGACCUC | 0_1-1_mismatch_A-C | | |
| | | CGUGCAUACU | 2_1-1_mismatch_C-C | | |
| | | ACGCAGCAUU | 4_2-2_bulge-symmetric_UU-UU | | |
| | | GGGAUACAGU | 8_1-1_wobble U-G | | |
| | | GUGAAAAGCA | 13_1-1_mismatch_A-G | | |
| | | GCA | 18_1-1_wobble_U-G | | |
| | | | 26_6-6_internal_loop-symmetric_GGGGAU-UAGGGG | | |
| | | | 34_1-1_wobble_G-U | | |
| 2719 | Ml_ | CCCUGGUGUG | −49_1-1_mismatch_C-A | −20 | |
| | Generative_ | CCCUCUGAUG | −20_6-6_internal_loop-symmetric_CAUCAU-UACUAC | | |
| | 0703 | UUUUUUAGGG | | | |
| | | GAUUCUACGG | | | |
| | | UAGGACUGAU | | | |
| | | CAGUCCCGUG | | | |
| | | GUCGAACCAC | | | |
| | | GUUGCAUACU | | | |
| | | ACGCAGCAUU | | | |
| | | GGGAUACAGU | | | |
| | | GUGAAAAGCA | | | |
| | | GCA | | | |
| 2720 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0719 | UUUUUUAGGG | −6_9-9_internal_loop-symmetric_AGAUUGCUG- | | |
| | | GAUUCUACGG | GGAACCCCG | | |
| | | CAAGACUGAG | −3_1-1_wobble_U-G | | |
| | | CAGUCCCGUG | 0_1-1_mismatch_A-C | | |
| | | GUGGAACCCC | 2_1-1_mismatch_C-C | | |
| | | GUUGCAUACU | 4_1-1_wobble_U-G | | |
| | | ACGCAGCAUU | 13_2-2_bulge-symmetric_AC-AG | | |
| | | GGGAUACAGU | 18_1-1_wobble_U-G | | |
| | | GUGAAAAGCA | 26_6-6_internal_loop-symmetric_GGGGAU-UAGGGG | | |
| | | GCA | 34_1-1_wobble_G-U | | |
| 2721 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0728 | UUUUUUAGGG | −11 4-5_internal_loop-asymmetric_AGAU-CCCCG | | |
| | | GAUCCUACGG | −7_1-1_wobble_U-G | | |
| | | CAGUACUGAU | −6_1-0_bulge-asymmetric_G- | | |
| | | CAAAUCCGUG | −3_1-1_wobble_U-G | | |
| | | GUGGCACCCC | 0_1-1_mismatch_A-C | | |
| | | GUUGCAUACU | 2_2-2_bulge-symmetric_CA-AU | | |
| | | ACGCAGCAUU | 7_1-1_mismatch_C-U | | |
| | | GGGAUACAGU | 18_1-1_wobble_U-G | | |
| | | GUGAAAAGCA | 23_1-1_mismatch_A-C | | |
| | | GCA | 26_6-6_internal_loop-symmetric_GGGGAU-UAGGGG | | |
| | | | 34_1-1_wobble_G-U | | |
| 2722 | Ml_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0732 | UUUUUUAGGG | −14_2-2_bulge-symmetric_AA-CG | | |
| | | GAUUCUACAG | −11_1-2_bulge-asymmetric_U-CC | | |
| | | CAGGACUGAC | −7_1-1_wobble_U-G | | |
| | | CAGUUCCGUG | −6_1-0_bulge-asymmetric_G- | | |
| | | GUGGCACCUC | −3_1-1_wobble_U-G | | |
| | | CGUGCAUACU | 0_1-1_mismatch_A-C | | |
| | | ACGCAGCAUU | 2_1-1_mismatch_C-U | | |
| | | GGGAUACAGU | 4_1-1_wobble_U-G | | |
| | | GUGAAAAGCA | 7_1-1_mismatch_C-C | | |
| | | GCA | 13_1-1_mismatch_A-G | | |
| | | | 26_6-6_internal_loop-symmetric_GGGGAU-UAGGGG | | |
| | | | 34_1-1_wobble_G-U | | |
| 2723 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0733 | UUUUUUAGGG | −11 4-5_internal_loop-asymmetric_AGAU-CCCCG | | |
| | | GAUUCUACGG | −7_1-1 wobble_U-G | | |
| | | CAGUACUGAG | −6_1-0_bulge-asymmetric_G- | | |
| | | AAAUCCCGUG | −3_1-1_wobble_U-G | | |
| | | GUGGCACCCC | 0_1-1_mismatch_A-C | | |
| | | GUUGCAUACU | 2_1-1_mismatch_C-C | | |
| | | ACGCAGCAUU | 6_1-1_mismatch_G-A | | |
| | | GGGAUACAGU | 18_1-1_wobble_U-G | | |
| | | GUGAAAAGCA | 26_6-6_internal_loop-symmetric_GGGGAU-UAGGGG | | |
| | | GCA | 34_1-1_wobble_G-U | | |
| 2724 | Ml_ | CCCUGGUGUG | −49_1-1_mismatch_C-A | −20 | 26 |
| | Generative_ | CCCUCUGAUG | −20_6-6_internal_loop-symmetric_CAUCAU-UACUAC | | |
| | 0742 | UUUUUUAGGG | −14_2-2_bulge-symmetric_AA-CG | | |
| | | GAUUCUACAG | −11_1-2_bulge-asymmetric_U-CC | | |
| | | CAGUCCUGAC | −9_1-1_wobble_G-U | | |
| | | CAGUCCCGUG | −7_1-0_bulge-asymmetric_U- | | |
| | | GUCGUACCUC | −3_1-1_wobble_U-G | | |
| | | CGUGCAUACU | 0_1-1_mismatch_A-C | | |
| | | ACGCAGCAUU | 2_1-1_mismatch_C-C | | |
| | | GGGAUACAGU | 4_1-1_wobble U-G | | |
| | | GUGAAAAGCA | 7_1-1_mismatch_C-C | | |
| | | GCA | 12_1-1_mismatch_U-C | | |
| | | | 26_6-6_internal_loop-symmetric_GGGGAU-UAGGGG | | |
| | | | 34_1-1_wobble_G-U | | |
| 2725 | Ml_ | CCCUGGUGUG | −49_1-1_mismatch_C-A | −20 | |
| | Generative_ | CCCUCUGAUG | −20_6-6_internal_loop-symmetric_CAUCAU-UACUAC | | |
| | 0743 | UUUUUUAGGG | | | |
| | | GCUUCUACAG | | | |
| | | CAGGACUGAU | | | |
| | | CAAAUCCGUG | | | |
| | | GUGGAACCCC | | | |
| | | GUUGCAUACU | | | |
| | | ACGCAGCAUU | | | |
| | | GGGAUACAGU | | | |
| | | GUGAAAAGCA | | | |
| | | GCA | | | |
| 2726 | Ml_ | CCCUGGUGUG | −49_1-1_mismatch_C-A | | |
| | Generative_ | CCCUCUGAUG | | | |
| | 0745 | UUUUUUAGGG | | | |
| | | GAUUCUACGG | | | |
| | | CAGUACUGAG | | | |
| | | ACAUCCCGUG | | | |
| | | GUGGCACCCC | | | |
| | | CGUGCAUACU | | | |
| | | ACGCAGCAUU | | | |
| | | GGGAUACAGU | | | |
| | | GUGAAAAGCA | | | |
| | | GCA | | | |
| 2727 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0766 | UUUUUUAGGG | −8_1-1_mismatch_C-U | | |
| | | GAUUCUACCG | −7_1-1_wobble_U-G | | |
| | | CAGUACUCAC | −3_1-0_bulge-asymmetric_U- | | |
| | | CAGUCGCCGU | 0_1-1_mismatch_A-C | | |
| | | GUCGUCAAUC | 2_0-1_bulge-asymmetric_-C | | |
| | | UUUGCAUACU | 4_1-1_wobble_U-G | | |
| | | ACGCAGCAUU | 7_3-3_bulge-symmetric_CUC-CAC | | |
| | | GGGAUACAGU | 18_1-1_mismatch_U-C | | |
| | | GUGAAAAGCA | 26_6-6_internal_loop-symmetric_GGGGAU-UAGGGG | | |
| | | GCA | 34_1-1_wobble_G-U | | |
| 2728 | Ml_ | CCCUGGUGUG | −49_1-1_mismatch_C-A | −20 | 26 |
| | Generative_ | CCCUCUGAUG | −20 6-6_internal_loop-symmetric_CAUCAU-UACUAC | | |
| | 0769 | UUUUUUAGGG | −7_1-1_wobble_U-G | | |
| | | GAUUCUACCG | −3_1-0_bulge-asymmetric_U- | | |
| | | CAGUACUCAC | 0_1-1_mismatch_A-C | | |
| | | CAGUCGCCGU | 2_0-1_bulge-asymmetric_-C | | |
| | | GUCGGCAAUC | 4_1-1_wobble_U-G | | |
| | | UUUGCAUACU | 7_3-3_bulge-symmetric_CUC-CAC | | |
| | | ACGCAGCAUU | 18_1-1_mismatch_U-C | | |
| | | GGGAUACAGU | 26_6-6_internal_loop-symmetric_GGGGAU-UAGGGG | | |
| | | GUGAAAAGCA | 34_1-1 wobble_G-U | | |
| | | GCA | | | |

The sequences for the engineered guides used as comparators in FIG. 44A-FIG. 44C is provided below in TABLE 10. While the engineered guide RNA sequences in TABLE 10 are provided as DNA sequences with a T substituted for each U, the corresponding RNA sequences are also encompassed herein.

TABLE 10
Comparator LRRK2 Engineered Guide RNA Sequences

| SEQ | | | | Left | Right |
|---|---|---|---|---|---|
| ID | | | Structural Features | Barbell | Barbell |
| NO | Guide Name | Sequence | (target/guide) | Position | Position |
| 2844 | A-C 0.113.57 | CCCTGGTGTGCCCTCT | −20_6-6_internal_loop- | −20 | 26 |
| | (−20, +26) | GATGTTTTTTAGGGG | symmetric_CAUCAU- | | |
| | | ATTCTACAGCAGTAC | UACUAC, 0_1-1_ | | |
| | | TGAGCAATGCCGTAG | mismatch_A-C, 26_6-6_ | | |
| | | TCAGCAATCTTTGCA | internal_loop- | | |
| | | TACTACGCAGCATTG | symmetric_GGGGAU- | | |
| | | GGATACAGTGTGAAA | UAGGGG | | |
| | | AGCAGCA | | | |
| 2845 | 1976 | CCCTGGTGTGCCCTCT | −20_6-6_internal_loop- | −20 | 26 |
| | 0.113.57 | GATGTTTTTTAGGGG | symmetric_CAUCAU- | | |
| | (−20, 26) | ATTCTACAACAGTAC | UACUAC, -8_1- | | |
| | | TGAGCTATCCCGAAT | 1_mismatch_C-A, −4- | | |
| | | TCAACAATCTTTGCA | >5_10-10_internal_loop- | | |
| | | TACTACGCAGCATTG | symmetric_CUACAGCAUU- | | |
| | | GGATACAGTGTGAAA | UAUCCCGAAU, 17_1- | | |
| | | AGCAGCA | 1_mismatch_C-A, 26_6- | | |
| | | | 6_internal_loop- | | |
| | | | symmetric_GGGGAU- | | |
| | | | UAGGGG | | |
| 2846 | 610 0.113.57 | CCCTGGTGTGCCCTCT | −20_6-6_internal_loop- | −20 | 26 |
| | (−20, 26) | GATGTTTTTTAGGGG | symmetric_CAUCAU- | | |
| | | ATTCTACATCTGTAGT | UACUAC, −4_1- | | |
| | | GAGCAATTCCGTGCT | 1_mismatch_C-C, −3_1- | | |
| | | CAGCAATCTTTGCAT | 1_wobble_U-G, 0_1- | | |
| | | ACTACGCAGCATTGG | 1_mismatch_A-C, 2_1- | | |
| | | GATACAGTGTGAAAA | 1_mismatch_C-U, 11_1- | | |
| | | GCAGCA | 1_mismatch_G-G, 15_1- | | |
| | | | 1_mismatch_U-U, 17_1- | | |
| | | | 1_mismatch_C-U, 26_6- | | |
| | | | 6_internal_loop- | | |
| | | | symmetric_GGGGAU- | | |
| | | | UAGGGG | | |

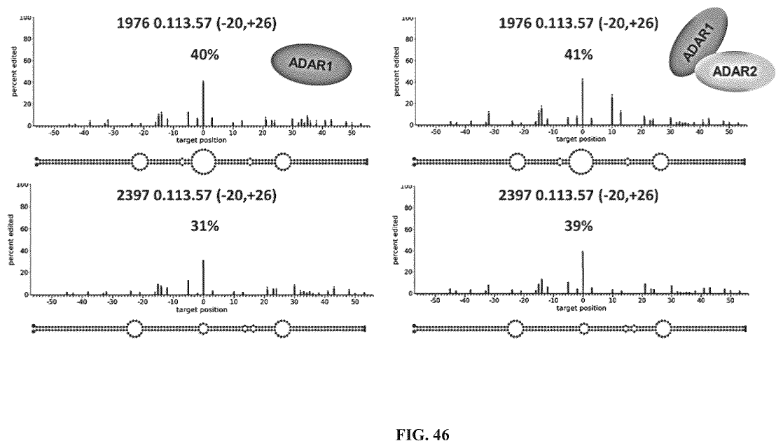

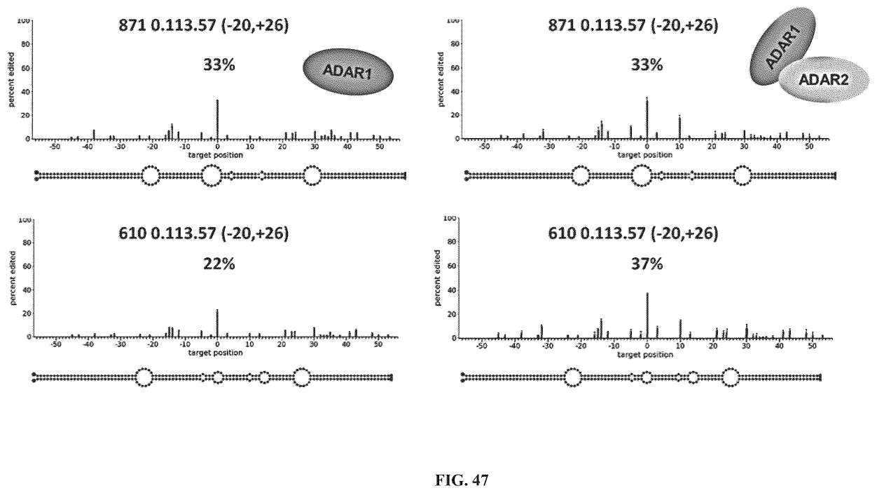

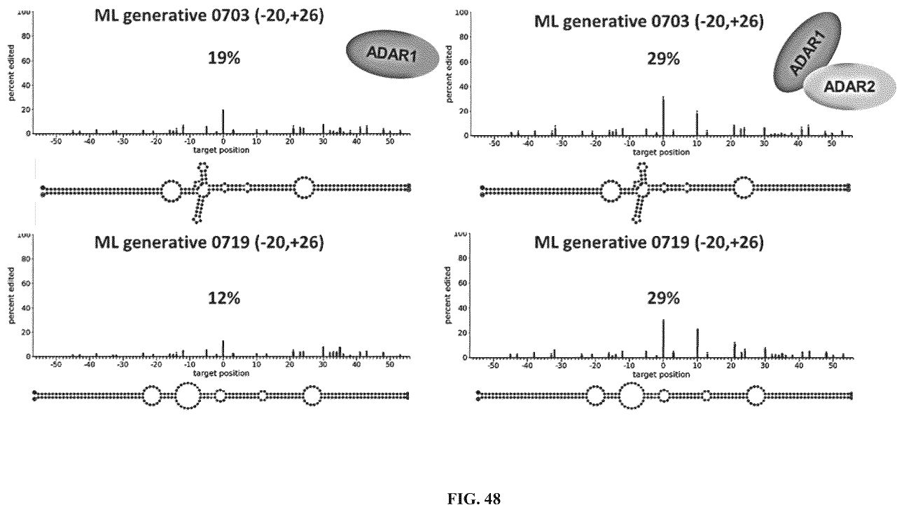

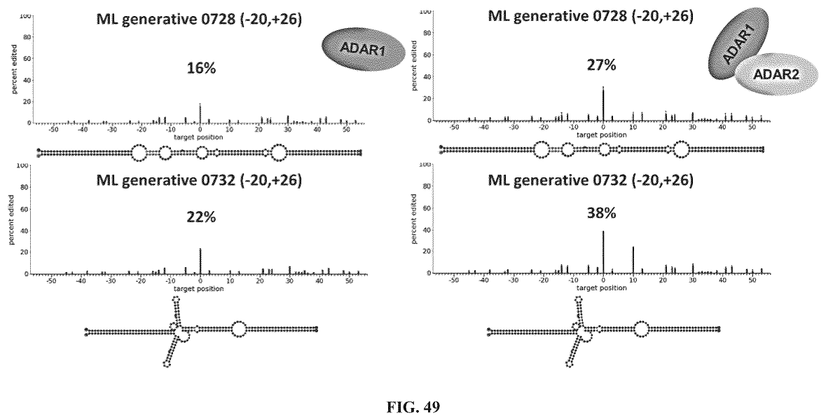

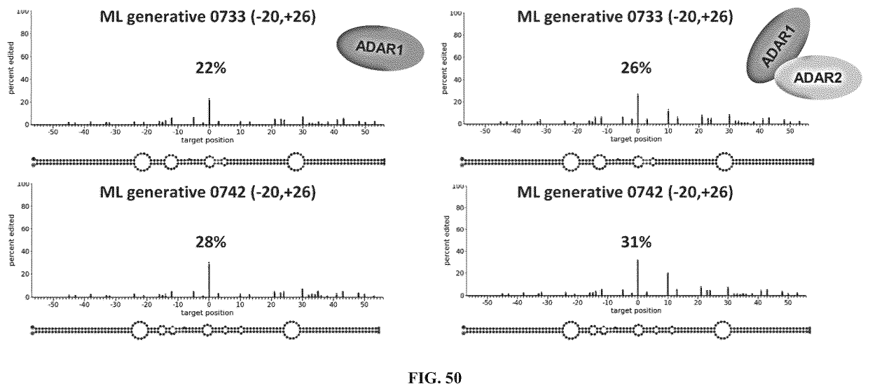

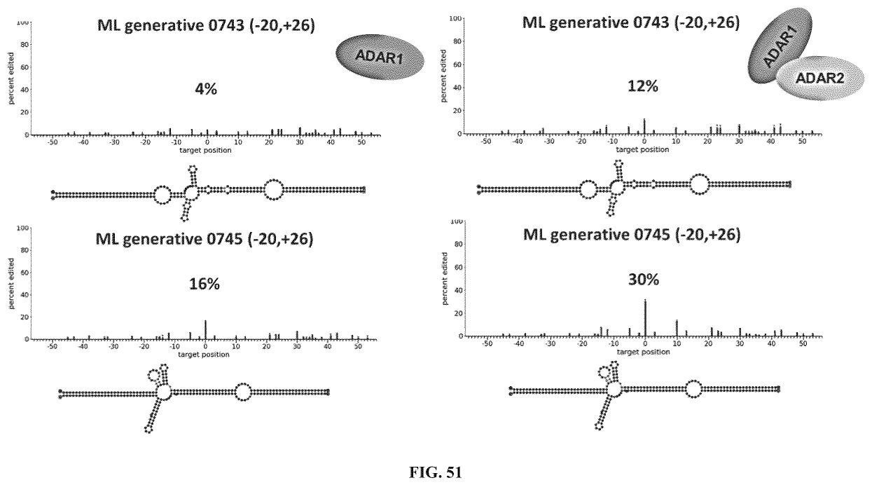

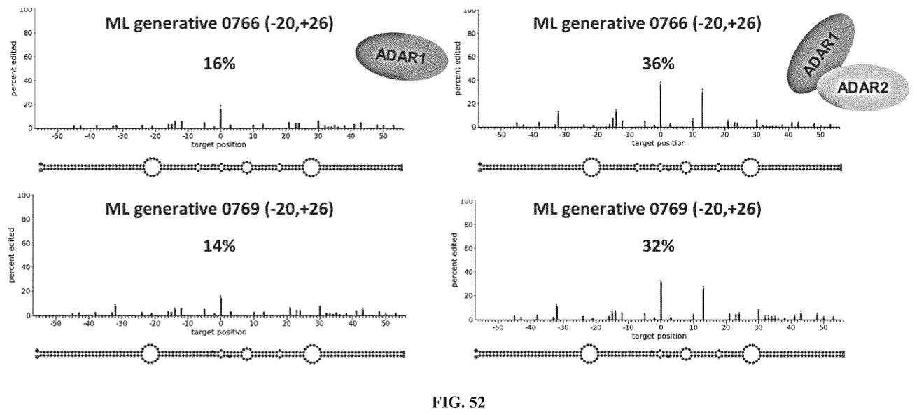

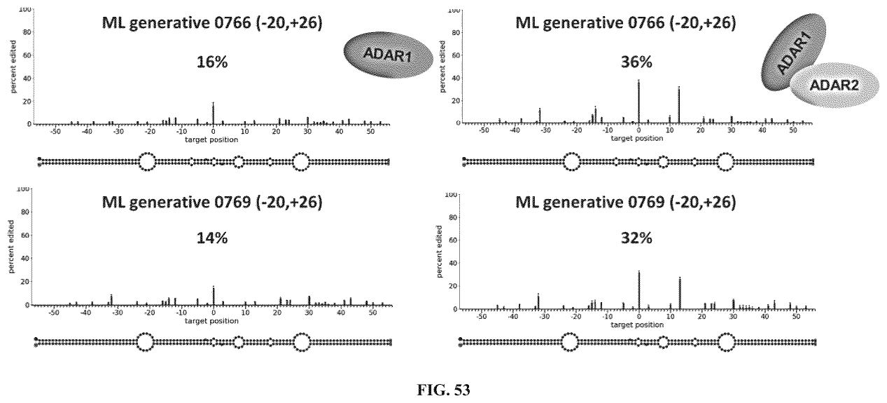

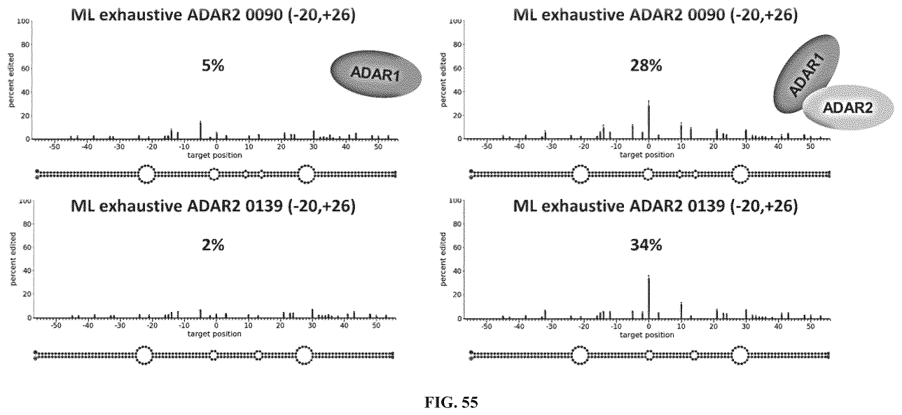

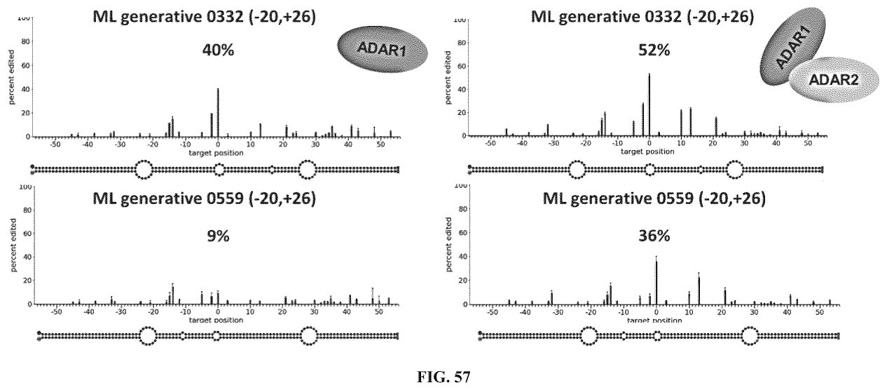

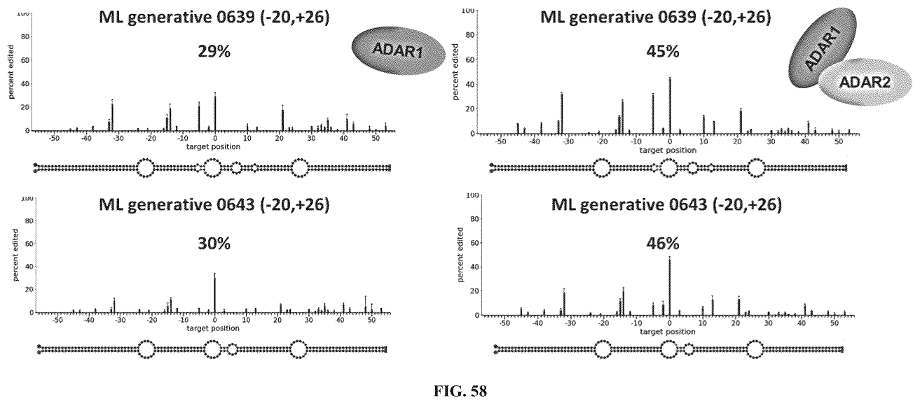

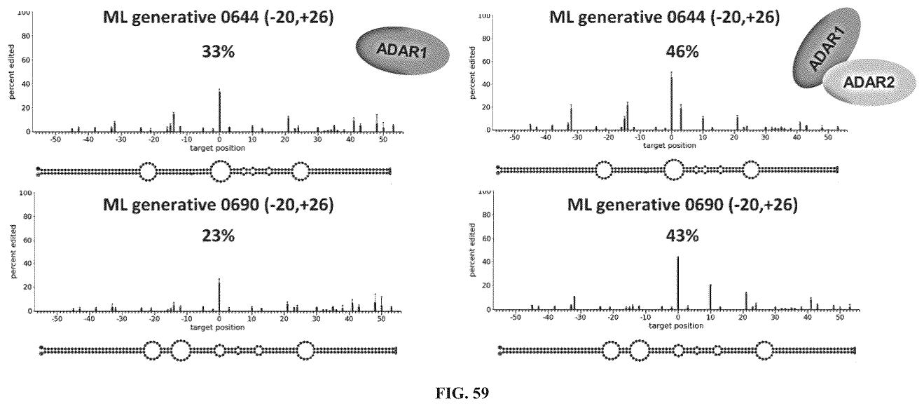

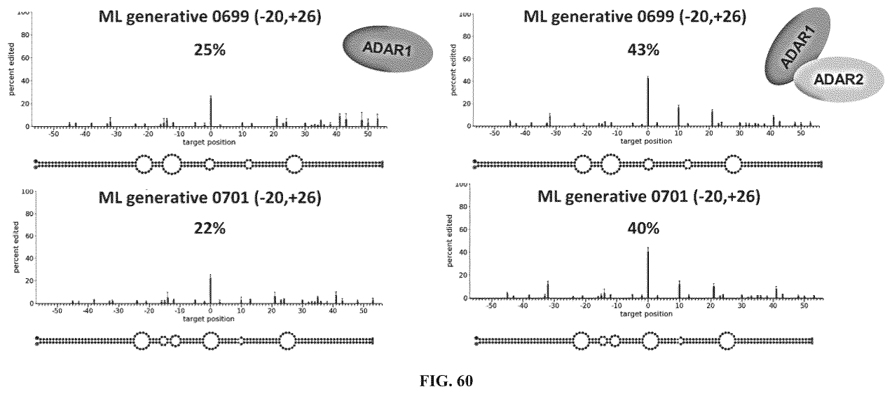

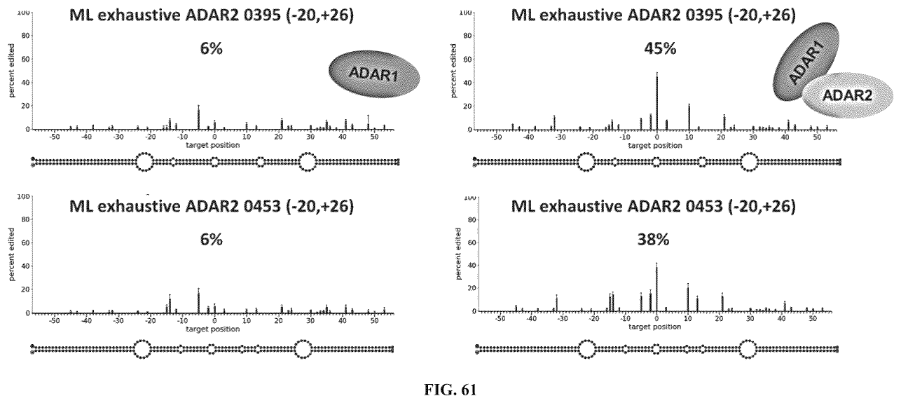

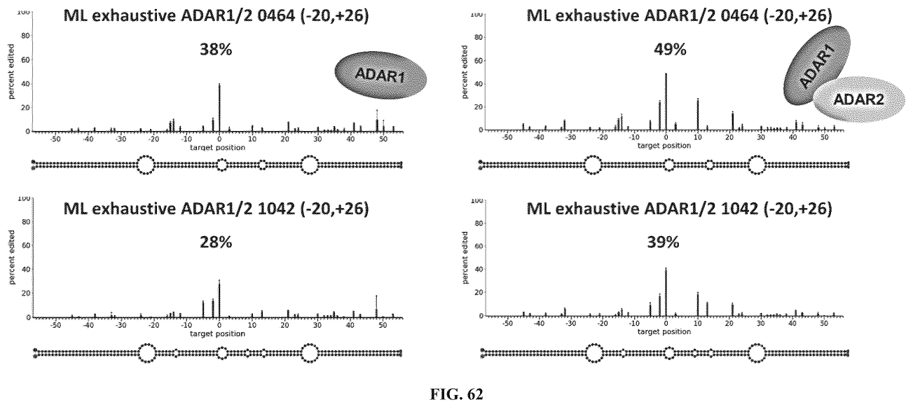

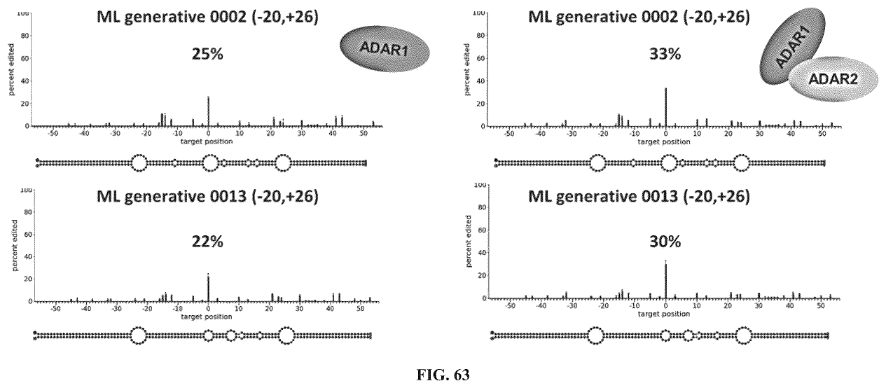

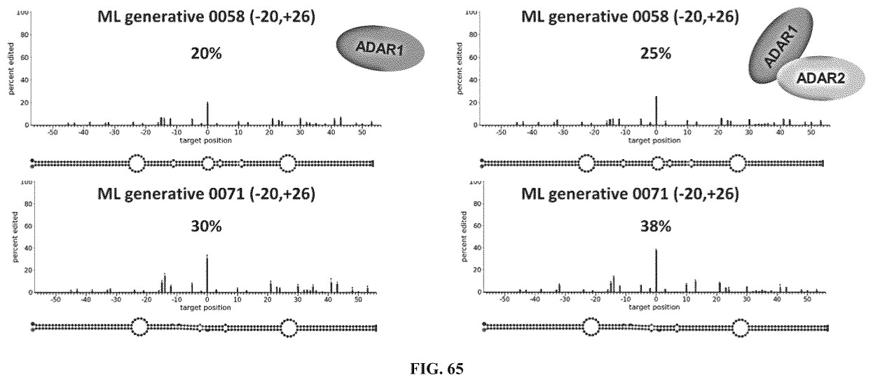

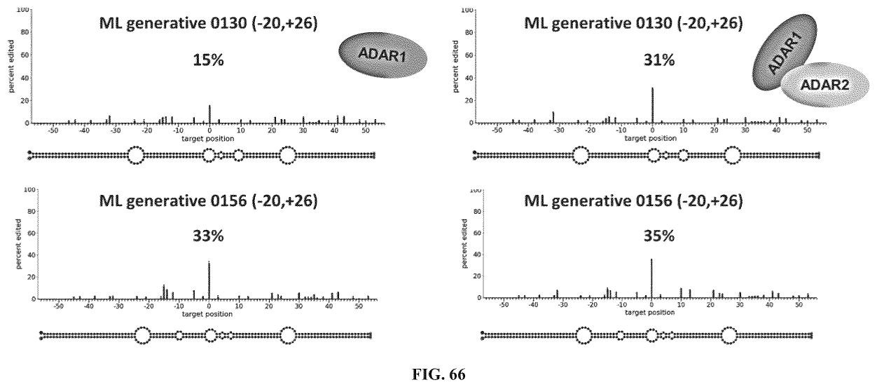

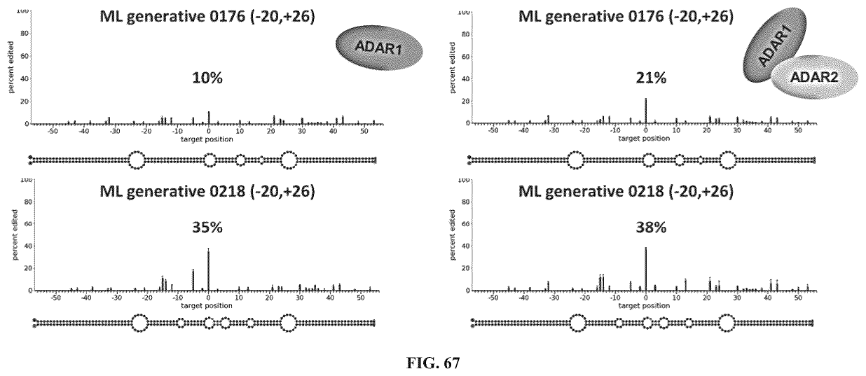

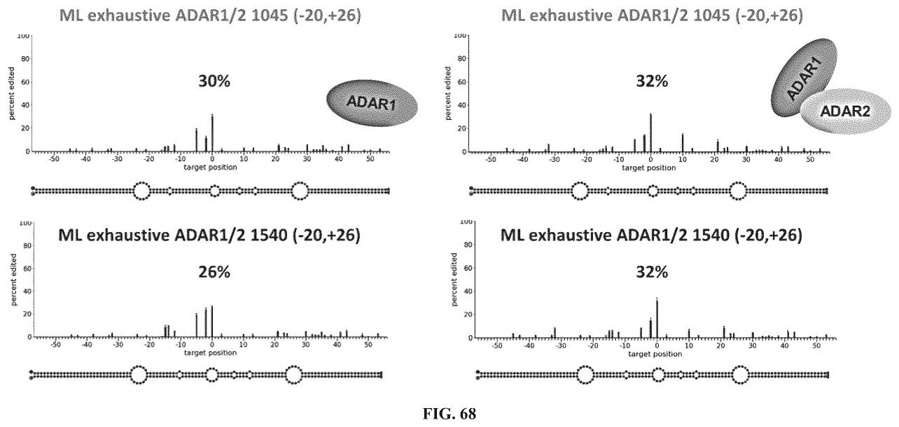

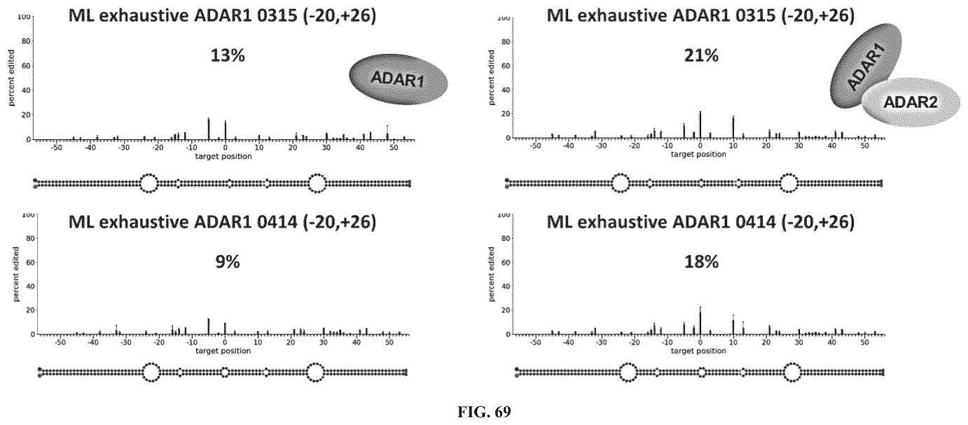

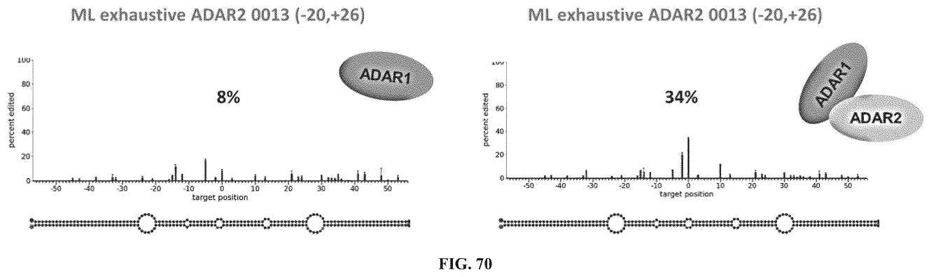

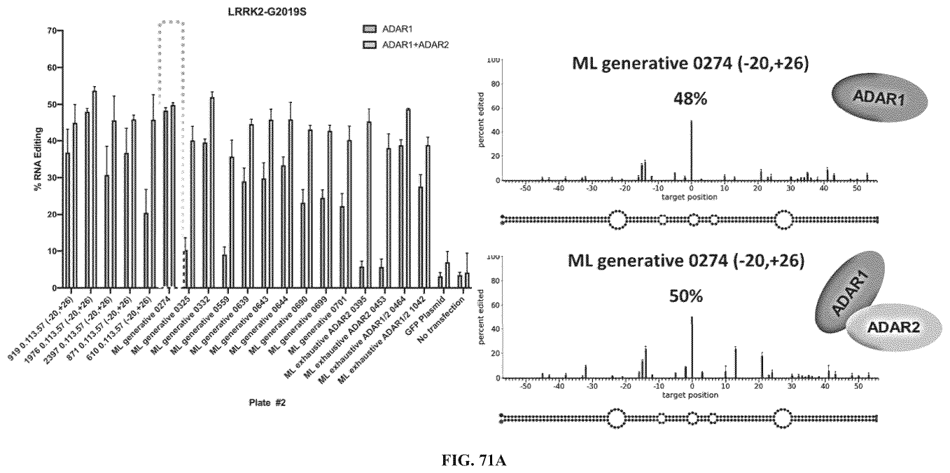

FIG. 45-FIG. 70 show the editing efficiency of each engineered guide RNA via either ADAR1-only or ADAR1+ADAR2. Engineered guide RNAs that facilitated superior editing via ADAR1 and ADAR1+ADAR2 were selected for further engineering. The following LRRK2 engineered guide RNAs recited in TABLE 11 correspond to the editing efficiency plots provided in FIG. 45-FIG. 70. While the engineered guide RNA sequences in TABLE 11 are provided as DNA sequences with a T substituted for each U, the corresponding RNA sequences are also encompassed herein.

TABLE 11
LRRK2 Engineered Guide RNA Sequences Utilized in Example 21

| SEQ | | | | | Left | Right |
|---|---|---|---|---|---|---|
| ID | | | | | Barbell | Barbell |
| NO | FIG. | Guide Name | Sequence | Structural Features (target/guide) | Position | Position |
| 2847 | 45 | A-C 0.113.57 | CCCTGGTGTGCCCTCTGATGTTTTTT | −20_6-6_internal_loop-symmetric_CAUCAU- | −20 | 26 |
| | | (−20, +26) | AGGGGATTCTACAGCAGTACTGAGC | UACUAC, 0_1-1_mismatch_A-C, 26_6- | | |
| | | | AATGCCGTAGTCAGCAATCTTTGCA | 6_internal_loop-symmetric_GGGGAU-UAGGGG | | |
| | | | TACTACGCAGCATTGGGATACAGTG | | | |
| | | | TGAAAAGCAGCA | | | |
| 2848 | 45 | 919 0.113.57 | CCCTGGTGTGCCCTCTGATGTTTTTT | −20_6-6_internal_loop-symmetric_CAUCAU- | −20 | 25 |
| | | (−20, +26) | AGGGGCTTCTACAGCAGTTCGGAGG | UACUAC, −3_1-1_wobble_U-G, −2_1- | | |
| | | | AATCCCGAGGTCAGCAATCTTTGCA | 1_mismatch_A-A, 0_1-1_mismatch_A-C, 2_1- | | |
| | | | TACTACGCAGCATTGGGATACAGTG | 1_mismatch_C-C, 6_1-1_mismatch_G-G, 10_3- | | |
| | | | TGAAAAGCAGCA | 3_bulge-symmetric_AGU-UCG, 25_7- | | |
| | | | | 7_internal_loop-symmetric_UGGGGAU- | | |
| | | | | UAGGGGC | | |
| 2849 | 46 | 1976 0.113.57 | CCCTGGTGTGCCCTCTGATGTTTTTT | −20_6-6_internal_loop-symmetric_CAUCAU- | −20 | 26 |
| | | (−20, +26) | AGGGGATTCTACAACAGTACTGAGC | UACUAC, −8_1-1_mismatch_C-A, −4→5_10- | | |
| | | | TATCCCGAATTCAACAATCTTTGCA | 10_internal_loop-symmetric_CUACAGCAUU- | | |
| | | | TACTACGCAGCATTGGGATACAGTG | UAUCCCGAAU, 17_1-1_mismatch_C-A, 26−6- | | |
| | | | TGAAAAGCAGCA | 6_internal_loop-symmetric_GGGGAU-UAGGGG | | |
| 2850 | 46 | 2397 0.113.57 | CCCTGGTGTGCCCTCTGATGTTTTTT | | −20 | 26 |
| | | (−20, +26) | AGGGGATTCTACCGCTGTGCTGGGC | | | |
| | | | AATCCCGTGGTCAGCAATCTTTGCA | | | |
| | | | TACTACGCAGCATTGGGATACAGTG | | | |
| | | | TGAAAAGCAGCA | | | |
| 2851 | 47 | 871 0.113.57 | CCCTGGTGTGCCCTCTGATGTTTTTT | −20_6-6_internal_loop-symmetric_CAUCAU- | −20 | 26 |
| | | (−20, +26) | AGGGGATTCTACAGTAGGACTGAGC | UACUAC, −7_1-1_wobble_U-G, −6_1- | | |
| | | | ACTGCCGAGCTGGGCAATCTTTGCA | 1_mismatch_G-G, −4_0-1_bulge-asymmetric_-C, | | |
| | | | TACTACGCAGCATTGGGATACAGTG | −2_1-0_bulge-asymmetric_A-, 0_1-1_mismatch_A- | | |
| | | | TGAAAAGCAGCA | C, 4_1-1_mismatch_U-C, 13_1-1_mismatch_A-G, | | |
| | | | | 16_1-1_wobble_G-U, 26_6-6_internal_loop- | | |
| | | | | symmetric_GGGGAU-UAGGGG | | |
| 2852 | 47 | 610 0.113.57 | CCCTGGTGTGCCCTCTGATGTTTTTT | −20_6-6_internal_loop-symmetric_CAUCAU- | −20 | 26 |
| | | (−20, +26) | AGGGGATTCTACATCTGTAGTGAGC | UACUAC, −4_1-1_mismatch_C-C, −3_1- | | |
| | | | AATTCCGTGCTCAGCAATCTTTGCA | 1_wobble_U-G, 0_1-1_mismatch_A-C, 2_1- | | |
| | | | TACTACGCAGCATTGGGATACAGTG | 1_mismatch_C-U, 11_1-1_mismatch_G-G, 15_1- | | |
| | | | TGAAAAGCAGCA | 1_mismatch_U-U, 17_1-1_mismatch_C-U, 26_6- | | |
| | | | | 6_internal_loop-symmetric_GGGGAU-UAGGGG | | |
| 2853 | 48 | ML generative | CCCTGGTGTGCCCTCTGATGTTTTTT | −20_6-6_internal_loop-symmetric_CAUCAU- | −20 | |
| | | 0703 | AGGGGATTCTACGGTAGGACTGATC | UACUAC | | |
| | | (−20, +26) | AGTCCCGTGGTCGAACCACGTTGCA | | | |
| | | | TACTACGCAGCATTGGGATACAGTG | | | |
| | | | TGAAAAGCAGCA | | | |
| 2854 | 48 | ML generative 0719 | CCCTGGTGTGCCCTCTGATGTTTTTT | −20_6-6_internal_loop-symmetric_CAUCAU- | −20 | 26 |
| | | (−20, +26) | AGGGGATTCTACGGCAAGACTGAGC | UACUAC, −6_9-9_internal_loop- | | |
| | | | AGTCCCGTGGTGGAACCCCGTTGCA | symmetric_AGAUUGCUG-GGAACCCCG, −3_1- | | |
| | | | TACTACGCAGCATTGGGATACAGTG | 1_wobble_U-G, 0_1-1_mismatch_A-C, 2_1- | | |
| | | | TGAAAAGCAGCA | 1_mismatch_C-C, 4_1-1_wobble U-G, 13_2- | | |
| | | | | 2_bulge-symmetric_AC-AG, 18_1-1_wobble_U-G, | | |
| | | | | 26_6-6_internal_loop-symmetric_GGGGAU- | | |
| | | | | UAGGGG | | |
| 2855 | 49 | ML generative 0728 | CCCTGGTGTGCCCTCTGATGTTTTTT | −20_6-6_internal_loop-symmetric_CAUCAU- | −20 | 26 |
| | | (−20, +26) | AGGGGATCCTACGGCAGTACTGATC | UACUAC, −11_4-5_internal_loop- | | |
| | | | AAATCCGTGGTGGCACCCCGTTGCA | asymmetric_AGAU-CCCCG, −7_1-1_wobble_U-G, | | |
| | | | TACTACGCAGCATTGGGATACAGTG | −6_1-0_bulge-asymmetric_G-, −3_1-1_wobble_U- | | |
| | | | TGAAAAGCAGCA | G, 0_1-1_mismatch_A-C, 2_2-2_bulge- | | |
| | | | | symmetric_CA-AU, 7_1-1_mismatch_C-U, 18_1- | | |
| | | | | 1_wobble_U-G, 23_1-1_mismatch_A-C, 26_6- | | |
| | | | | 6_internal_loop-symmetric_GGGGAU-UAGGGG | | |
| 2856 | 49 | ML generative 0732 | CCCTGGTGTGCCCTCTGATGTTTTTT | −20_6-6_internal_loop-symmetric_CAUCAU- | −20 | 26 |
| | | (−20, +26) | AGGGGATTCTACAGCAGGACTGACC | UACUAC, −14_2-2_bulge-symmetric_AA-CG, | | |
| | | | AGTTCCGTGGTGGCACCTCCGTGCA | −11_1-2_bulge-asymmetric_U-CC, -7_1- | | |
| | | | TACTACGCAGCATTGGGATACAGTG | 1_wobble_U-G, −6_1-0_bulge-asymmetric_G-, | | |
| | | | TGAAAAGCAGCA | −3_1-1_wobble_U-G, 0_1-1_mismatch_A-C, 2_1- | | |
| | | | | 1_mismatch_C-U, 4_1-1_wobble_U-G, 7_1- | | |
| | | | | 1_mismatch_C-C, 13_1-1_mismatch_A-G, 26_6- | | |
| | | | | 6_internal_loop-symmetric_GGGGAU-UAGGGG | | |
| 2857 | 50 | ML_generative_0733 | CCCTGGTGTGCCCTCTGATGTTTTTT | −20_6-6_internal_loop-symmetric_CAUCAU- | −20 | 26 |
| | | (−20, +26) | AGGGGATTCTACGGCAGTACTGAGA | UACUAC, −11_4-5_internal_loop- | | |
| | | | AATCCCGTGGTGGCACCCCGTTGCA | asymmetric_AGAU-CCCCG, −7_1-1_wobble_U-G, | | |
| | | | TACTACGCAGCATTGGGATACAGTG | −6_1-0_bulge-asymmetric_G-, −3_1-1_wobble_U- | | |
| | | | TGAAAAGCAGCA | G, 0_1-1_mismatch_A-C, 2_1-1_mismatch_C-C, | | |
| | | | | 6_1-1_mismatch_G-A, 18_1-1_wobble_U-G, 26_6- | | |
| | | | | 6_internal_loop-symmetric_GGGGAU-UAGGGG | | |
| 2858 | 50 | ML_generative_0742 | CCCTGGTGTGCCCTCTGATGTTTTTT | −20_6-6_internal_loop-symmetric_CAUCAU- | −20 | 26 |
| | | (−20, +26) | AGGGGATTCTACAGCAGTCCTGACC | UACUAC, −14_2-2_bulge-symmetric_AA-CG, | | |
| | | | AGTCCCGTGGTCGTACCTCCGTGCA | −11_1-2_bulge-asymmetric_U-CC, −9_1- | | |
| | | | TACTACGCAGCATTGGGATACAGTG | 1_wobble_G-U, −7_1-0_bulge-asymmetric_U-, | | |
| | | | TGAAAAGCAGCA | −3_1-1_wobble_U-G, 0_1-1_mismatch_A-C, 2_1- | | |
| | | | | 1_mismatch_C-C, 4_1-1_wobble_U-G, 7_1- | | |
| | | | | 1_mismatch_C-C, 12_1-1_mismatch_U-C, 26_6- | | |
| | | | | 6_internal_loop-symmetric_GGGGAU-UAGGGG | | |
| 2859 | 51 | ML_generative_0743 | CCCTGGTGTGCCCTCTGATGTTTTTT | −20_6-6_internal_loop-symmetric_CAUCAU- | −20 | |
| | | (−20, +26) | AGGGGCTTCTACAGCAGGACTGATC | UACUAC | | |
| | | | AAATCCGTGGTGGAACCCCGTTGCA | | | |
| | | | TACTACGCAGCATTGGGATACAGTG | | | |
| | | | TGAAAAGCAGCA | | | |
| 2860 | 51 | ML_generative_0745 | CCCTGGTGTGCCCTCTGATGTTTTTT | | | |
| | | (−20, +26) | AGGGGATTCTACGGCAGTACTGAGA | | | |
| | | | CATCCCGTGGTGGCACCCCCGTGCA | | | |
| | | | TACTACGCAGCATTGGGATACAGTG | | | |
| | | | TGAAAAGCAGCA | | | |
| 2861 | 52 | ML_generative_0766 | CCCTGGTGTGCCCTCTGATGTTTTTT | −20_6-6_internal_loop-symmetric_CAUCAU- | −20 | 26 |
| | 53 | (−20, +26) | AGGGGATTCTACCGCAGTACTCACC | UACUAC, −8_1-1_mismatch_C-U, −7_1- | | |
| | | | AGTCGCCGTGTCGTCAATCTTTGCA | 1_wobble_U-G, −3_1-0_bulge-asymmetric_U-, | | |
| | | | TACTACGCAGCATTGGGATACAGTG | 0_1-1_mismatch_A-C, 2_0-1_bulge-asymmetric_- | | |
| | | | TGAAAAGCAGCA | C, 4_1-1_wobble_U-G, 7_3-3_bulge- | | |
| | | | | symmetric_CUC-CAC, 18_1-1_mismatch_U-C, | | |
| | | | | 26_6-6_internal_loop-symmetric_GGGGAU- | | |
| | | | | UAGGGG | | |
| 2862 | 52 | ML_generative_0769 | CCCTGGTGTGCCCTCTGATGTTTTTT | −20_6-6_internal_loop-symmetric_CAUCAU- | −20 | 26 |
| | 53 | (−20, +26) | AGGGGATTCTACCGCAGTACTCACC | UACUAC, −7_1-1_wobble_U-G, −3_1-0_bulge- | | |
| | | | AGTCGCCGTGTCGGCAATCTTTGCA | asymmetric_U-, 0_1-1_mismatch_A-C, 2_0- | | |
| | | | TACTACGCAGCATTGGGATACAGTG | 1_bulge-asymmetric_-C, 4_1-1_wobble_U-G, 7_3- | | |
| | | | TGAAAAGCAGCA | 3_bulge-symmetric_CUC-CAC, 18_1- | | |
| | | | | 1_mismatch_U-C, 26_6-6_internal_loop- | | |
| | | | | symmetric_GGGGAU-UAGGGG | | |
| 2863 | 54 | ML_exhaustive_ADA | CCCTGGTGTGCCCTCTGATGTTTTTT | −20_6-6_internal_loop-symmetric_CAUCAU- | −20 | 26 |
| | 73 | R2_0049 (−20, +26) | AGGGGATTCTACAGCATTACTCAGC | UACUAC, −11_1-1_mismatch_U-C, −1→0_2- | | |
| | | | AGTGCGTTAGTCAGCACTCTTTGCA | 2_bulge-symmetric_CA-GU, 4_1-1_wobble_U-G, | | |
| | | | TACTACGCAGCATTGGGATACAGTG | 9_1-1_mismatch_C-C, 14_1-1_mismatch_C-U, | | |
| | | | TGAAAAGCAGCA | 26_6-6_internal_loop-symmetric_GGGGAU- | | |
| | | | | UAGGGG | | |
| 2864 | 54 | ML_exhaustive_ADA | CCCTGGTGTGCCCTCTGATGTTTTTT | −20_6-6_internal_loop-symmetric_CAUCAU- | −20 | 26 |
| | 73 | R2_0069 (−20, +26) | AGGGGATTCTACAGCACGACTGAGC | UACUAC, −7_1-1_wobble_U-G, −1→0_2-2_bulge- | | |
| | | | AGTGCGTTAGTCGGCAATCTTTGCA | symmetric_CA-GU, 4_1-1_wobble U-G, 13_2- | | |
| | | | TACTACGCAGCATTGGGATACAGTG | 2_bulge-symmetric_AC-CG, 26_6-6_internal_loop- | | |
| | | | TGAAAAGCAGCA | symmetric_GGGGAU-UAGGGG | | |
| 2865 | 55 | ML_exhaustive_ADA | CCCTGGTGTGCCCTCTGATGTTTTTT | −20_6-6_internal_loop-symmetric_CAUCAU- | −20 | 26 |
| | 73 | R2_0090 (−20, +26) | AGGGGATTCTACAGCAATACTCAGC | UACUAC, −2→0_3-3_bulge-symmetric_ACA- | | |
| | | | AGTGCGTGAGTCAGCAATCTTTGCA | GUG, 4_1-1_wobble_U-G, 9_1-1_mismatch_C-C, | | |
| | | | TACTACGCAGCATTGGGATACAGTG | 14_1-1_mismatch_C-A, 26_6-6_internal_loop- | | |
| | | | TGAAAAGCAGCA | symmetric_GGGGAU-UAGGGG | | |
| 2866 | 55 | ML exhaustive_ADA | CCCTGGTGTGCCCTCTGATGTTTTTT | −20_6-6_internal_loop-symmetric_CAUCAU- | −20 | 26 |
| | 73 | R2_0139 (−20, +26) | AGGGGATTCTACAGCACGACTGAGC | UACUAC, −7_1-1_wobble_U-G, −1→0_2-2_bulge- | | |
| | | | AGTGCGCTAGTCGGCAATCTTTGCA | symmetric_CA-GC, 4_1-1_wobble_U-G, 13_2- | | |
| | | | TACTACGCAGCATTGGGATACAGTG | 2_bulge-symmetric_AC-CG, 26_6-6_internal_loop- | | |
| | | | TGAAAAGCAGCA | symmetric_GGGGAU-UAGGGG | | |
| 2867 | 56 | ML_generative_0274 | CCCTGGTGTGCCCTCTGATGTTTTTT | −20_6-6_internal_loop-symmetric_CAUCAU- | −20 | 26 |
| | | (−20, +26) | AGGGGATTCTACAGCAGTACTGTCC | UACUAC, −8_2-2_bulge-symmetric_GC-UA, | | |
| | | | AGTCCCGTGGTCGTAAATCTTTGCA | −7_1-1_wobble_U-G, −3_1-1_wobble_U-G, 0_1- | | |
| | | | TACTACGCAGCATTGGGATACAGTG | 1_mismatch_A-C, 2_1-1_mismatch_C-C, 4_1- | | |
| | | | TGAAAAGCAGCA | 1_wobble_U-G, 7_2-2_bulge-symmetric_CU-UC, | | |
| | | | | 26_6-6_internal_loop-symmetric_GGGGAU- | | |
| | | | | UAGGGG | | |
| 2868 | 56 | ML_generative_0325 | CCCTGGTGTGCCCTCTGATGTTTTTT | −20_6-6_internal_loop-symmetric_CAUCAU- | −20 | 26 |
| | | (−20, +26) | AGGGGATTCTACATCAGGACTGAGC | UACUAC, −10_1-1_mismatch_U-U, −7_1- | | |
| | | | TTTTCCGAGGTCGGCTATCTTTGCAT | 1_wobble_U-G, −3_1-1_wobble_U-G, −2→5_8- | | |
| | | | ACTACGCAGCATTGGGATACAGTGT | 8_internal_loop-symmetric_ACAGCAUU- | | |
| | | | GAAAAGCAGCA | UUUUCCGA, 13_1-1_mismatch_A-G, 17_1- | | |
| | | | | 1_mismatch_C-U, 26_6-6_internal_loop- | | |
| | | | | symmetric_GGGGAU-UAGGGG | | |
| 2869 | 57 | ML_generative_0332 | CCCTGGTGTGCCCTCTGATGTTTTTT | −20_6-6_internal_loop-symmetric_CAUCAU- | −20 | 26 |
| | | (−20, +26) | AGGGGATTCTACATCAGTACTGGGC | UACUAC, −7_1-1_wobble_U-G, 0_1- | | |
| | | | AGTTCCGTAGTCGGCAATCTTTGCA | 1_mismatch_A-C, 2_1-1_mismatch_C-U, 4_1- | | |
| | | | TACTACGCAGCATTGGGATACAGTG | 1_wobble_U-G, 8_1-1_wobble_U-G, 17_1- | | |
| | | | TGAAAAGCAGCA | 1_mismatch_C-U, 26_6-6_internal_loop- | | |
| | | | | symmetric_GGGGAU-UAGGGG | | |
| 2870 | 57 | ML_generative_0559 | CCCTGGTGTGCCCTCTGATGTTTTTT | −20_6-6_internal_loop-symmetric_CAUCAU- | −20 | 26 |
| | | (−20, +26) | AGGGGATTCTACAGCAGTACTGGGC | UACUAC, −11_1-1_mismatch_U-C, −7_1- | | |
| | | | AGTGCGCTAGTCGGCACTCTTTGCA | 1_wobble_U-G, −1→0_2-2_bulge-symmetric_CA- | | |
| | | | TACTACGCAGCATTGGGATACAGTG | GC, 4_1-1_wobble_U-G, 8_1-1_wobble_U-G, | | |
| | | | TGAAAAGCAGCA | 26_6-6_internal_loop-symmetric_GGGGAU- | | |
| | | | | UAGGGG | | |
| 2871 | 58 | ML_generative_0639 | CCCTGGTGTGCCCTCTGATGTTTTTT | −20_6-6_internal_loop-symmetric_CAUCAU- | −20 | 26 |
| | | (−20, +26) | AGGGGATTCTACAGCAATACTCACC | UACUAC, −6_1-1_mismatch_G-A, −3→2_6- | | |
| | | | AGTCCCGATGTAAGCAATCTTTGCA | 6_internal_loop-symmetric_UACAGC-CCCGAU, | | |
| | | | TACTACGCAGCATTGGGATACAGTG | 4_1-1_wobble_U-G, 7_3-3_bulge- | | |
| | | | TGAAAAGCAGCA | symmetric_CUC-CAC, 14_1-1_mismatch_C-A, | | |
| | | | | 26_6-6_internal_loop-symmetric_GGGGAU- | | |
| | | | | UAGGGG | | |
| 2872 | 58 | ML_generative_0643 | CCCTGGTGTGCCCTCTGATGTTTTTT | −20_6-6_internal_loop-symmetric_CAUCAU- | −20 | 26 |
| | | (−20, +26) | AGGGGATTCTACGGCAGTACTGTGA | UACUAC, −7_1-1_wobble_U-G, −3→6_10- | | |
| | | | AATCCCGATGTCGGCAATCTTTGCA | 10_internal_loop-symmetric_UACAGCAUUG- | | |
| | | | TACTACGCAGCATTGGGATACAGTG | AAAUCCCGAU, 8_1-1_mismatch_U-U, 18_1- | | |
| | | | TGAAAAGCAGCA | 1_wobble_U-G, 26_6-6_internal_loop- | | |
| | | | | symmetric_GGGGAU-UAGGGG | | |
| 2873 | 59 | ML_generative_0644 | CCCTGGTGTGCCCTCTGATGTTTTTT | −20_6-6_internal_loop-symmetric_CAUCAU- | −20 | 26 |
| | | (−20, +26) | AGGGGATTCTACCGCAGGACGGAG | UACUAC, −7_0-1_bulge-asymmetric_-G, −6_1- | | |
| | | | CTATCCCGAGTTAGGCAATCTTTGC | 1_wobble_G-U, −2→5_8-7_internal_loop- | | |
| | | | ATACTACGCAGCATTGGGATACAGT | asymmetric_ACAGCAUU-UAUCCCG, 10_1- | | |
| | | | GTGAAAAGCAGCA | 1_mismatch_A-G, 13_1-1_mismatch_A-G, 18_1- | | |
| | | | | 1_mismatch_U-C, 26_6-6_internal_loop- | | |
| | | | | symmetric_GGGGAU-UAGGGG | | |
| 2874 | 59 | ML_generative_0690 | CCCTGGTGTGCCCTCTGATGTTTTTT | −20_6-6_internal_loop-symmetric_CAUCAU- | −20 | 26 |
| | | (−20, +26) | AGGGGATTCTACGGCATGACTGATC | UACUAC, −14_2-2_bulge-symmetric_AA-GA, | | |
| | | | AGTCCCGTGGTCGGACCCCGATGCA | −9_4-4_bulge-symmetric_AUUG-ACCC, −7_1- | | |
| | | | TACTACGCAGCATTGGGATACAGTG | 1_wobble_U-G, −3_1-1_wobble_U-G, 0_1- | | |
| | | | TGAAAAGCAGCA | 1_mismatch_A-C, 2_1-1_mismatch_C-C, 4_1- | | |
| | | | | 1_wobble_U-G, 7_1-1_mismatch_C-U, 13_2- | | |
| | | | | 2_bulge-symmetric_AC-UG, 18_1-1_wobble_U-G, | | |
| | | | | 26_6-6_internal_loop-symmetric_GGGGAU- | | |
| | | | | UAGGGG | | |
| 2875 | 60 | ML_generative_0699 | CCCTGGTGTGCCCTCTGATGTTTTTT | −20_6-6_internal_loop-symmetric_CAUCAU- | −20 | 26 |
| | | (−20, +26) | AGGGGATTCTACGGCAAGACTGGGC | UACUAC, −9_7-7_internal_loop- | | |
| | | | AGTCCCGTGGTCGGACCCCCGTGCA | symmetric_AAGAUUG-ACCCCCG, −7_1- | | |
| | | | TACTACGCAGCATTGGGATACAGTG | 1_wobble_U-G, -3_1-1_wobble_U-G, 0_1- | | |
| | | | TGAAAAGCAGCA | 1_mismatch_A-C, 2_1-1_mismatch_C-C, 4_1- | | |
| | | | | 1_wobble_U-G, 8_1-1_wobble_U-G, 13_2- | | |
| | | | | 2_bulge-symmetric_AC-AG, 18_1-1_wobble_U-G, | | |
| | | | | 26_6-6_internal_loop-symmetric_GGGGAU- | | |
| | | | | UAGGGG | | |
| 2876 | 60 | ML_generative_0701 | CCCTGGTGTGCCCTCTGATGTTTTTT | −20_6-6_internal_loop-symmetric_CAUCAU- | −20 | 26 |
| | | (−20, +26) | AGGGGATTCTACGGCAGGACTGGGC | UACUAC, −14_2-2_bulge-symmetric_AA-CG, | | |
| | | | TTTCCCGTGGTCGGACCTCCGTGCA | −9_3-3_bulge-symmetric_UUG-ACC, −7_1- | | |
| | | | TACTACGCAGCATTGGGATACAGTG | 1_wobble_U-G, −3_1-1_wobble_U-G, 0_1- | | |
| | | | TGAAAAGCAGCA | 1_mismatch_A-C, 2_1-1_mismatch_C-C, 4_2- | | |
| | | | | 2_bulge-symmetric_UU-UU, 8_1-1_wobble_U-G, | | |
| | | | | 13_1-1_mismatch_A-G, 18_1-1_wobble_U-G, | | |
| | | | | 26_6-6_internal_loop-symmetric_GGGGAU- | | |
| | | | | UAGGGG | | |
| 2877 | 61 | ML_exhaustive_ADA | CCCTGGTGTGCCCTCTGATGTTTTTT | −20_6-6_internal_loop-symmetric_CAUCAU- | −20 | 26 |
| | 73 | R2_0395 (−20, +26) | AGGGGATTCTACAGCACGACTGAGC | UACUAC, −13_1-1_mismatch_G-A, −1→0_2- | | |
| | | | AGTGCGCTAGTCAGCAATATTTGCA | 2_bulge-symmetric_CA-GC, 4_1-1_wobble_U-G, | | |
| | | | TACTACGCAGCATTGGGATACAGTG | 13_2-2_bulge-symmetric_AC-CG, 26_6- | | |
| | | | TGAAAAGCAGCA | 6_internal_loop-symmetric_GGGGAU-UAGGGG | | |
| 2878 | 61 | ML_exhaustive_ADA | CCCTGGTGTGCCCTCTGATGTTTTTT | −20_6-6_internal_loop-symmetric_CAUCAU- | −20 | 26 |
| | 73 | R2_0453 (−20, +26) | AGGGGATTCTACAGCAATACTCAGC | UACUAC, −10_1-1_mismatch_U-C, −1→0_2- | | |
| | | | AGTGCGCTAGTCAGCCATCTTTGCA | 2_bulge-symmetric_CA-GC, 4_1-1_wobble_U-G, | | |
| | | | TACTACGCAGCATTGGGATACAGTG | 9_1-1_mismatch_C-C, 14_1-1_mismatch_C-A, | | |
| | | | TGAAAAGCAGCA | 26_6-6_internal_loop-symmetric_GGGGAU- | | |
| | | | | UAGGGG | | |
| 2879 | 62 | ML_exhaustive_ADA | CCCTGGTGTGCCCTCTGATGTTTTTT | −20_6-6_internal_loop-symmetric_CAUCAU- | −20 | 26 |
| | | R1/2_0464 | AGGGGATTCTACAGCATGACTGAGC | UACUAC, −7_1-1_wobble_U-G, 0_1- | | |
| | | (−20, +26) | AGTCCCGTAGTCGGCAATCTTTGCA | 1_mismatch_A-C, 2_1-1_mismatch_C-C, 4_1- | | |
| | | | TACTACGCAGCATTGGGATACAGTG | 1_wobble_U-G, 13_2-2_bulge-symmetric_AC-UG, | | |
| | | | TGAAAAGCAGCA | 26_6-6_internal_loop-symmetric_GGGGAU- | | |
| | | | | UAGGGG | | |
| 2880 | 62 | ML exhaustive_ADA | CCCTGGTGTGCCCTCTGATGTTTTTT | −20_6-6_internal_loop-symmetric_CAUCAU- | −20 | 26 |
| | | R1/2_1042 | AGGGGATTCTACAGCAATACTCAGC | UACUAC, −13_1-1_mismatch_G-A, 0_1- | | |
| | | (−20, +26) | AGTTCCGTAGTCAGCAATATTTGCA | 1_mismatch_A-C, 2_1-1_mismatch_C-U, 4_1- | | |
| | | | TACTACGCAGCATTGGGATACAGTG | 1_wobble_U-G, 9_1-1_mismatch_C-C, 14_1- | | |
| | | | TGAAAAGCAGCA | 1_mismatch_C-A, 26_6-6_internal_loop- | | |
| | | | | symmetric_GGGGAU-UAGGGG | | |
| 2881 | 63 | ML_generative_0002 | CCCTGGTGTGCCCTCTGATGTTTTTT | −20_6-6_internal_loop-symmetric_CAUCAU- | −20 | 26 |
| | | (−20, +26) | AGGGGATTCTAGAGAGGTACTGTGC | UACUAC, −10_1-1_mismatch_U-C, −7_1- | | |
| | | | CATCCCGTGGTCGGCCATCTTTGCA | 1_wobble_U-G, -3_1-1_wobble_U-G, 0_1- | | |
| | | | TACTACGCAGCATTGGGATACAGTG | 1_mismatch_A-C, 2_1-1_mismatch_C-C, 5_1- | | |
| | | | TGAAAAGCAGCA | 1_mismatch_U-C, 8_1-1_mismatch_U-U, 15_1- | | |
| | | | | 1_wobble_U-G, 16_1-1_mismatch_G-A, 19_1- | | |
| | | | | 1_mismatch_G-G, 26_6-6_internal_loop- | | |
| | | | | symmetric_GGGGAU-UAGGGG | | |
| 2882 | 63 | ML_generative_0013 | CCCTGGTGTGCCCTCTGATGTTTTTT | −20_6-6_internal_loop-symmetric_CAUCAU- | −20 | 26 |
| | | (−20, +26) | AGGGGATTCTAGAGCAGGACGGTG | UACUAC, −7_1-1_wobble_U-G, −3_1- | | |
| | | | CAGTCCCGTGGTCGGCAATCTTTGC | 1_wobble_U-G, 0_1-1_mismatch_A-C, 2_1- | | |
| | | | ATACTACGCAGCATTGGGATACAGT | 1_mismatch_C-C, 4_1-1_wobble_U-G, 8_1- | | |
| | | | GTGAAAAGCAGCA | 1_mismatch_U-U, 10_1-1_mismatch_A-G, 13_1- | | |
| | | | | 1_mismatch_A-G, 19_1-1_mismatch_G-G, 26_6- | | |
| | | | | 6_internal_loop-symmetric_GGGGAU-UAGGGG | | |
| 2883 | 64 | ML_generative_0016 | CCCTGGTGTGCCCTCTGATGTTTTTT | −20_6-6_internal_loop-symmetric_CAUCAU- | −20 | 26 |
| | | (−20, +26) | AGGGGATTCTACAGGAGGACTGGG | UACUAC, −10_2-2_bulge-symmetric_UU-CU, | | |
| | | | CAGTCCCGTGGTCGCCCTTCTTTGCA | −8_1-1_mismatch_C-C, −7_1-1_wobble_U-G, −3_1- | | |
| | | | TACTACGCAGCATTGGGATACAGTG | 1_wobble_U-G, 0_1-1_mismatch_A-C, 2_1- | | |
| | | | TGAAAAGCAGCA | 1_mismatch_C-C, 4_1-1_wobble_U-G, 8_1- | | |
| | | | | 1_wobble_U-G, 13_1-1_mismatch_A-G, 16_1- | | |
| | | | | 1_mismatch_G-G, 26_6-6_internal_loop- | | |
| | | | | symmetric_GGGGAU-UAGGGG | | |
| 2884 | 64 | ML_generative_0043 | CCCTGGTGTGCCCTCTGATGTTTTTT | −20_6-6_internal_loop-symmetric_CAUCAU- | −20 | 26 |
| | | (−20, +26) | AGGGGATTCTACAGCGGTACTGTGC | UACUAC, −10_1-1_mismatch_U-C, −9_1- | | |
| | | | AAATCCGTGGTCGGTCATCTTTGCA | 1_wobble_G-U, −7_1-1_wobble_U-G, −3_1- | | |
| | | | TACTACGCAGCATTGGGATACAGTG | 1_wobble_U-G, 0_1-1_mismatch_A-C, 2_2- | | |
| | | | TGAAAAGCAGCA | 2_bulge-symmetric_CA-AU, 8_1-1_mismatch_U- | | |
| | | | | U, 15_1-1_wobble_U-G, 26_6-6_internal_loop- | | |
| | | | | symmetric_GGGGAU-UAGGGG | | |
| 2885 | 65 | ML_generative_0058 | CCCTGGTGTGCCCTCTGATGTTTTTT | −20_6-6_internal_loop-symmetric_CAUCAU- | −20 | 26 |
| | | (−20, +26) | AGGGGATTCTACGGCAGGACTGGG | UACUAC, −10_1-1_mismatch_U-C, −7_1- | | |
| | | | GAAATCCGTGGTCGGCCATCTTTGC | 1_wobble_U-G, −3_1-1_wobble_U-G, 0_1- | | |
| | | | ATACTACGCAGCATTGGGATACAGT | 1_mismatch_A-C, 2_2-2_bulge-symmetric_CA- | | |
| | | | GTGAAAAGCAGCA | AU, 6_1-1_mismatch_G-G, 8_1-1_wobble_U-G, | | |
| | | | | 13_1-1_mismatch_A-G, 18_1-1_wobble_U-G, | | |
| | | | | 26_6-6_internal_loop-symmetric_GGGGAU- | | |
| | | | | UAGGGG | | |
| 2886 | 65 | ML_generative_0071 | CCCTGGTGTGCCCTCTGATGTTTTTT | −20_6-6_internal_loop-symmetric_CAUCAU- | −20 | 26 |
| | | (−20, +26) | AGGGGATTCTACAGCGGTACTGCGC | UACUAC, −10_1-1_mismatch_U-C, −8_1- | | |
| | | | AGTCCCGTGGTGGTCCATCTTTGCA | 1_mismatch_C-U, −7_1-1_wobble_U-G, −6_1- | | |
| | | | TACTACGCAGCATTGGGATACAGTG | 1_mismatch_G-G, −3_1-1_wobble_U-G, 0_1- | | |
| | | | TGAAAAGCAGCA | 1_mismatch_A-C, 2_1-1_mismatch_C-C, 4_1- | | |
| | | | | wobble_U-G, 8_1-1_mismatch_U-C, 15_1- | | |
| | | | | 1_wobble_U-G, 26_6-6_internal_loop- | | |
| | | | | symmetric_GGGGAU-UAGGGG | | |
| 2887 | 66 | ML_generative_0130 | CCCTGGTGTGCCCTCTGATGTTTTTT | −20_6-6_internal_loop-symmetric_CAUCAU- | −20 | 26 |
| | | (−20, +26) | AGGGGATTCTATAGCAGGAATGAG | UACUAC, −7_1-1_wobble_U-G, −3_1- | | |
| | | | GAAATCCGTGGTCGGCAATCTTTGC | 1_wobble_U-G, 0_1-1_mismatch_A-C, 2_2- | | |
| | | | ATACTACGCAGCATTGGGATACAGT | 2_bulge-symmetric_CA-AU, 6_1-1_mismatch_G- | | |
| | | | GTGAAAAGCAGCA | G, 11_3-3_bulge-symmetric_GUA-GAA, 19_1- | | |
| | | | | 1_wobble_G-U, 26_6-6_internal_loop- | | |
| | | | | symmetric_GGGGAU-UAGGGG | | |
| 2888 | 66 | ML_generative_0156 | CCCTGGTGTGCCCTCTGATGTTTTTT | −20_6-6_internal_loop-symmetric_CAUCAU- | −20 | 26 |
| | | (−20, +26) | AGGGGATTCTACAGCAGTACTCAGG | UACUAC, −9_2-2_bulge-symmetric_UG-AU, | | |
| | | | AAATCCGTGGTCGGATATCTTTGCA | −7_1-1_wobble_U-G, −3_1-1_wobble_U-G, 0_1- | | |
| | | | TACTACGCAGCATTGGGATACAGTG | 1_mismatch_A-C, 2_2-2_bulge-symmetric_CA- | | |
| | | | TGAAAAGCAGCA | AU, 6_1-1_mismatch_G-G, 9_1-1_mismatch_C-C, | | |
| | | | | 26_6-6_internal_loop-symmetric_GGGGAU- | | |
| | | | | UAGGGG | | |
| 2889 | 67 | ML_generative_0176 | CCCTGGTGTGCCCTCTGATGTTTTTT | −20_6-6_internal_loop-symmetric_CAUCAU- | −20 | 26 |
| | | (−20, +26) | AGGGGATTCTAAAGCAGGAGTGAG | UACUAC, −7_1-1_wobble_U-G, −3_1- | | |
| | | | CAGATCCGTGGTCGGCAATCTTTGC | 1_wobble_U-G, 0→3_4-4_bulge- | | |
| | | | ATACTACGCAGCATTGGGATACAGT | symmetric_AGCA-AUCC, 4_1-1_wobble_U-G, | | |
| | | | GTGAAAAGCAGCA | 11_1-1_mismatch_G-G, 13_1-1_mismatch_A-G, | | |
| | | | | 19_1-1_mismatch_G-A, 26_6-6_internal_loop- | | |
| | | | | symmetric_GGGGAU-UAGGGG | | |
| 2890 | 67 | ML_generative_0218 | CCCTGGTGTGCCCTCTGATGTTTTTT | −20_6-6_internal_loop-symmetric_CAUCAU- | −20 | 26 |
| | | (−20, +26) | AGGGGATTCTACAGGTGTACTGTGA | UACUAC, −8_2-2_bulge-symmetric_GC-UA, | | |
| | | | AATCCCGTGGTCATAAATCTTTGCA | −3_1-1_wobble_U-G, 0_1-1_mismatch_A-C, 2_1- | | |
| | | | TACTACGCAGCATTGGGATACAGTG | 1_mismatch_C-C, 6_1-1_mismatch_G-A, 8_1- | | |
| | | | TGAAAAGCAGCA | 1_mismatch_U-U, 15_2-2_bulge-symmetric_UG- | | |
| | | | | GU, 26_6-6_internal_loop-symmetric_GGGGAU- | | |
| | | | | UAGGGG | | |
| 2891 | 68 | ML_exhaustive_ADA | CCCTGGTGTGCCCTCTGATGTTTTTT | −20_6-6_internal_loop-symmetric_CAUCAU- | −20 | 26 |
| | | R1/2_1045 | AGGGGATTCTACAGCATTACTCAGC | UACUAC, −13_1-1_mismatch_G-A, 0_1- | | |
| | | (−20, +26) | AGTCCCGTAGTCAGCAATATTTGCA | 1_mismatch_A-C, 2_1-1_mismatch_C-C, 4_1- | | |
| | | | TACTACGCAGCATTGGGATACAGTG | 1_wobble_U-G, 9_1-1_mismatch_C-C, 14_1- | | |
| | | | TGAAAAGCAGCA | 1_mismatch_C-U, 26_6-6_internal_loop- | | |
| | | | | symmetric_GGGGAU-UAGGGG | | |
| 2892 | 68 | ML_exhaustive_ADA | CCCTGGTGTGCCCTCTGATGTTTTTT | −20_6-6_internal_loop-symmetric_CAUCAU- | −20 | 26 |
| | | R1/2_1540 | AGGGGATTCTACAGCATTACTCAGC | UACUAC, −9_1-1_mismatch_G-A, 0_1- | | |
| | | (−20, +26) | AAATCCGTAGTCAGAAATCTTTGCA | 1_mismatch_A-C, 2_2-2_bulge-symmetric_CA- | | |
| | | | TACTACGCAGCATTGGGATACAGTG | AU, 9_1-1_mismatch_C-C, 14_1-1_mismatch_C- | | |
| | | | TGAAAAGCAGCA | U, 26_6-6_internal_loop-symmetric_GGGGAU- | | |
| | | | | UAGGGG | | |
| 2893 | 69 | ML_exhaustive_ADA | CCCTGGTGTGCCCTCTGATGTTTTTT | −20_6-6_internal_loop-symmetric_CAUCAU- | −20 | 26 |
| | | R1_0315 (−20, +26) | AGGGGATTCTACAGCAGGACTGAGC | UACUAC, −13_1-1_mismatch_G-A, −3_1- | | |
| | | | AATCTTGTGGTCAGCAATATTTGCA | 1_wobble_U-G, 1_1-1_wobble_G-U, 2_1- | | |
| | | | TACTACGCAGCATTGGGATACAGTG | 1_mismatch_C-C, 13_1-1_mismatch_A-G, 26_6- | | |
| | | | TGAAAAGCAGCA | 6_internal_loop-symmetric_GGGGAU-UAGGGG | | |
| 2894 | 69 | ML_exhaustive_ADA | CCCTGGTGTGCCCTCTGATGTTTTTT | −20_6-6_internal_loop-symmetric_CAUCAU- | −20 | 26 |
| | | R1_0414 (−20, +26) | AGGGGATTCTACAGCAGGACTGAGC | UACUAC, −13_1-1_mismatch_G-A, −3_1- | | |
| | | | AATGGAGTGGTCAGCAATATTTGCA | 1_wobble_U-G, 0→1_2-2_bulge-symmetric_AG- | | |
| | | | TACTACGCAGCATTGGGATACAGTG | GA, 13_1-1_mismatch_A-G, 26_6-6_internal_loop- | | |
| | | | TGAAAAGCAGCA | symmetric_GGGGAU-UAGGGG | | |
| 2895 | 70 | ML_exhaustive_ADA | CCCTGGTGTGCCCTCTGATGTTTTTT | −20_6-6_internal_loop-symmetric_CAUCAU- | −20 | 26 |
| | 73 | R2_0013 (−20, +26) | AGGGGATTCTACAGCACGACTGAGC | UACUAC, −10_1-1_mismatch_U-C, −1→0_2- | | |
| | | | AGTGCGTTAGTCAGCCATCTTTGCA | 2_bulge-symmetric_CA-GU, 4_1-1_wobble_U-G, | | |
| | | | TACTACGCAGCATTGGGATACAGTG | 13_2-2_bulge-symmetric_AC-CG, 26_6- | | |
| | | | TGAAAAGCAGCA | 6_internal_loop-symmetric_GGGGAU-UAGGGG | | |

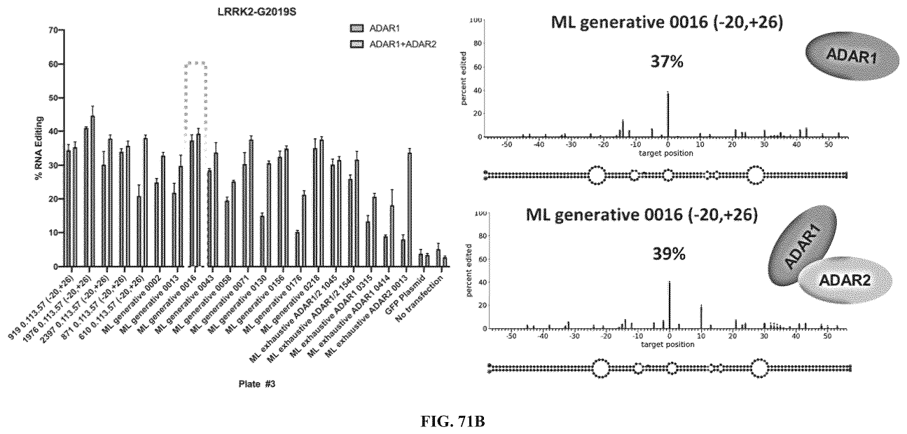

FIG. 71A and FIG. 71B shows selection of two exemplary engineered guide RNAs displaying superior editing that were selected for further engineering.

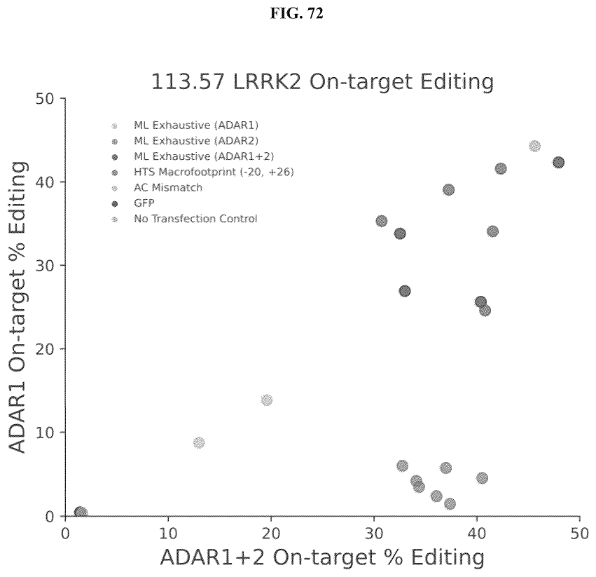

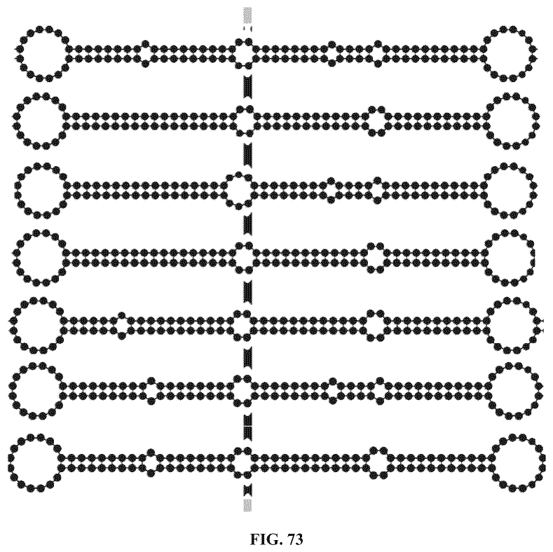

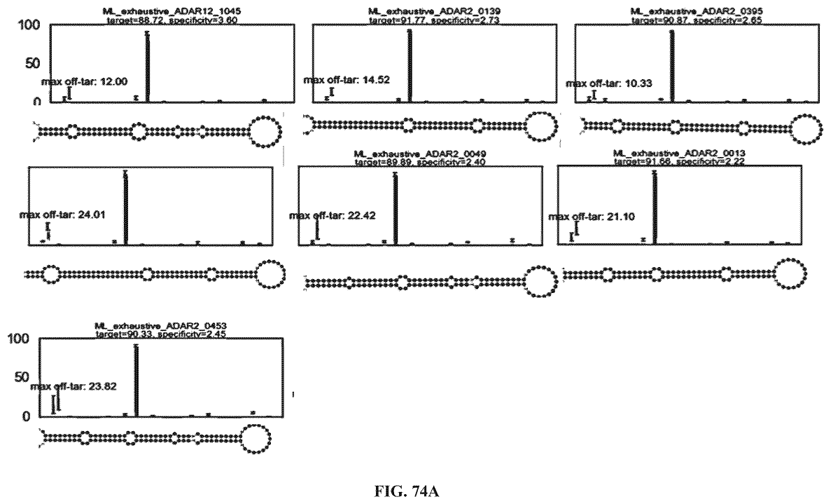

Of note, the machine learning algorithm provided an accurate prediction of ADAR specificity. As shown in FIG. 72, guide RNAs selected for ADAR1, ADAR2, or ADAR1 and ADAR2 displayed specificity for the appropriate ADAR enzyme in vitro. FIG. 73 depicts engineered guide RNA designs that showed specificity for ADAR2 in FIG. 72. In this system, engineered guide RNAs designed to form an A-G mismatch at the target adenosine exhibited facilitating preferential RNA editing by ADAR2.

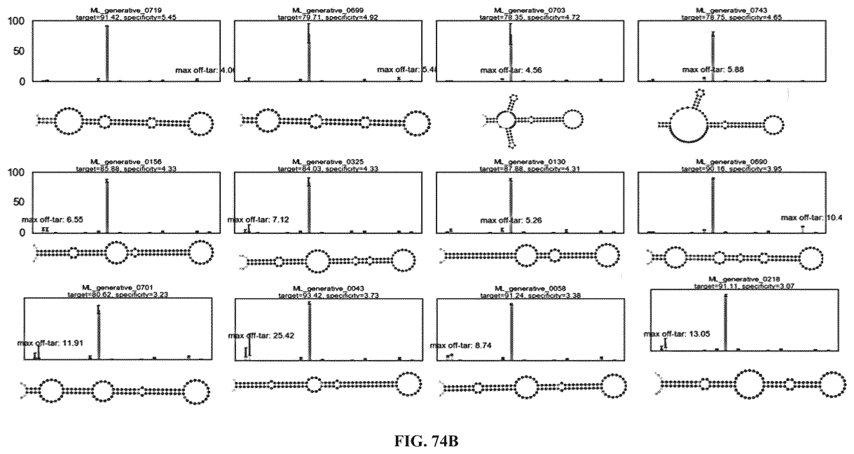

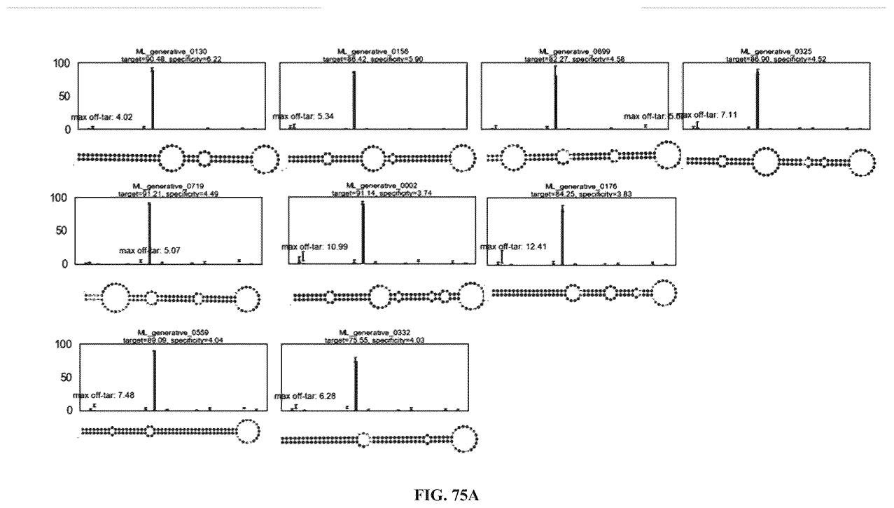

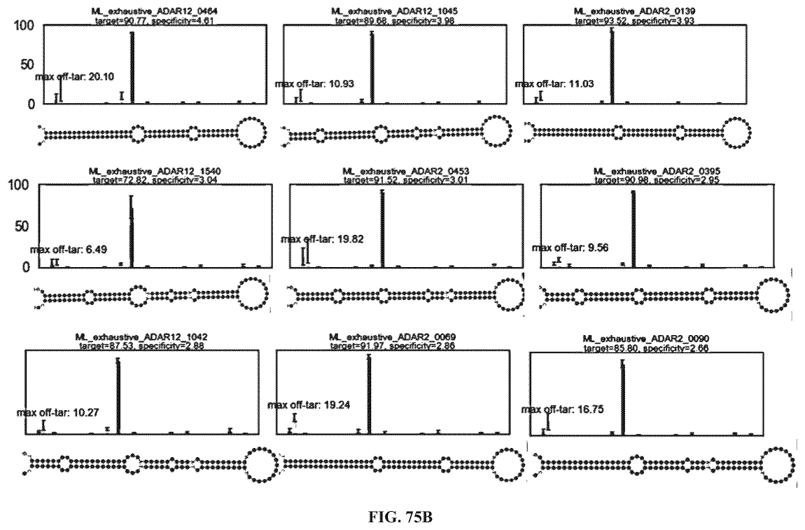

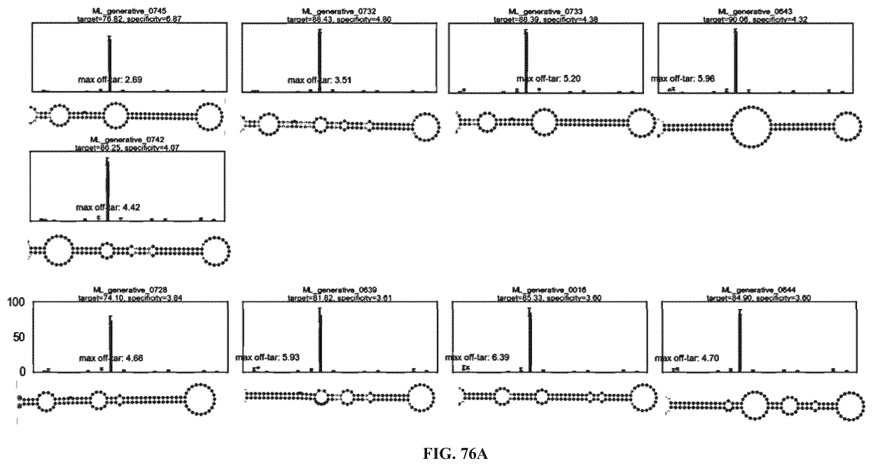

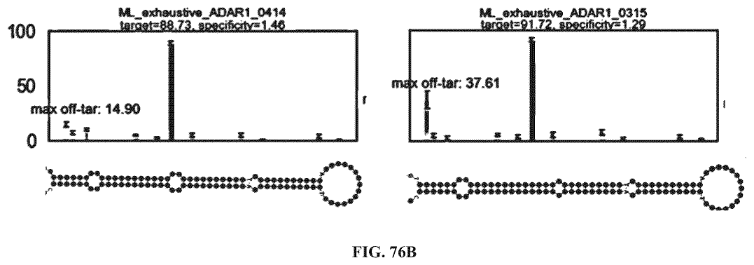

FIG. 74A and FIG. 74B show the top performing engineered guide RNAs that display specificity for ADAR1+ADAR2. FIG. 75A and FIG. 75B show the top performing engineered guide RNAs that display specificity for ADAR2. FIG. 76A and FIG. 76B show the top performing engineered guide RNAs that display specificity for ADAR1.

### Example 22

ML gRNAs Targeting LRRK2

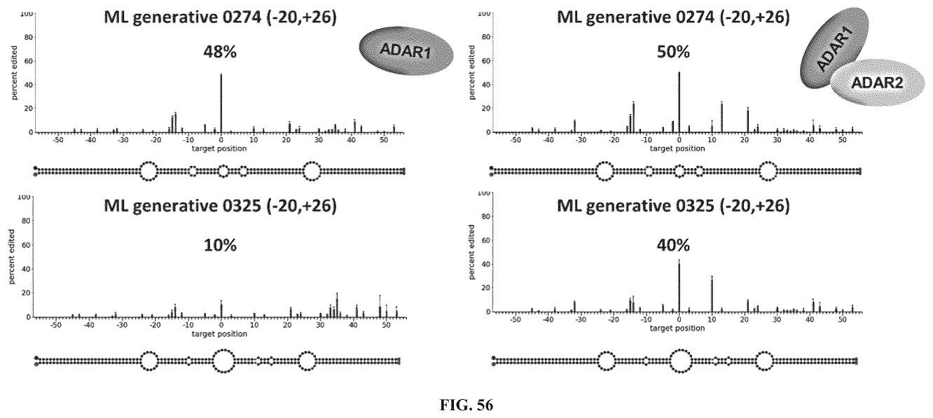

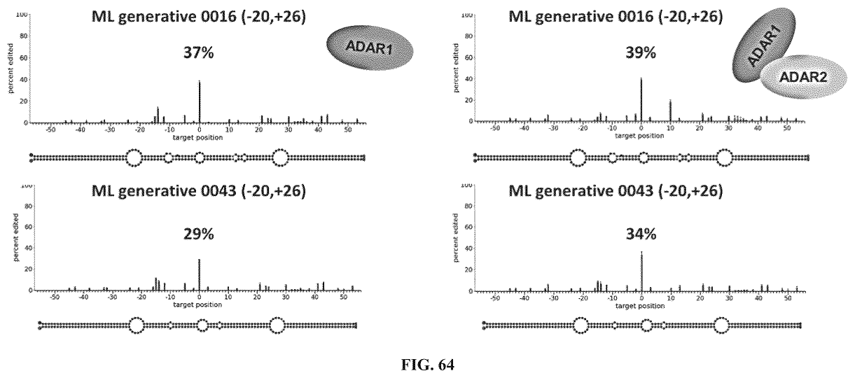

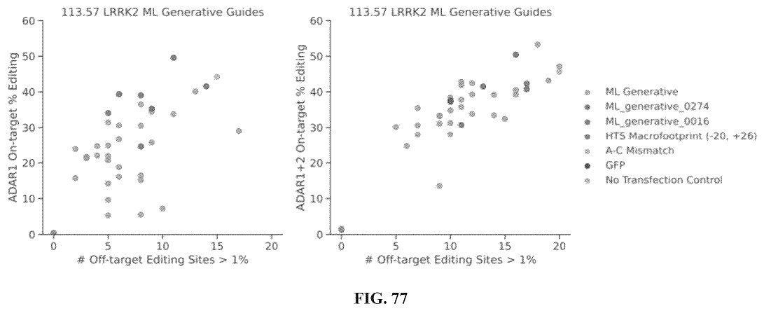

This example describes machine learning (ML)-derived gRNAs targeting LRRK2. Two machine learning model types were utilized, a generative model and an exhaustive model, to engineer LRRK2 gRNAs that were subsequently evaluated. Next-generation sequencing (NGS) was used to compare highly efficient and specific ML-derived gRNAs and gRNAs generated using in vitro high throughput screening (HTS) methods. gRNAs were dosed in HEK293 cells expressing a LRRK2 cDNA minigene. Two generative ML gRNAs, in particular, leveraged ADAR to facilitate highly efficient and specific RNA editing (FIG. 64—CCCTGGTGTGCCCTCTGATGTTTTTTAGGGGATTCTACAGGAGGACTGGGCAGTCCCGTGGTCGC CCTTCTTTGCATACTACGCAGCATTGGGATACAGTGTGAAAAGCAGCA (SEQ ID NO: 2769), FIG. 56—CCCTGGTGTGCCCTCTGATGTTTTTTAGGGGATTCTACAGCAGTACTGTCCAGTCCCGTGGTCGT AAATCTTTGCATACTACGCAGCATTGGGATACAGTGTGAAAAGCAGCA (SEQ ID NO: 2770), and FIG. 77).

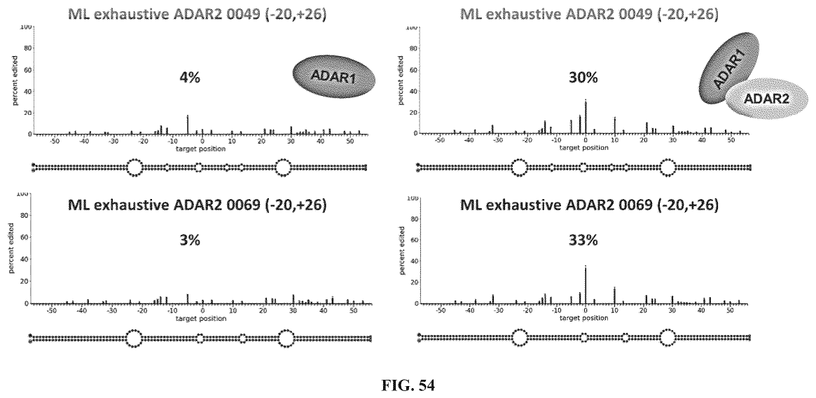

In addition, gRNAs that preferentially leverage ADAR2 for RNA editing are also disclosed herein (e.g., FIG. 54—CCCTGGTGTGCCCTCTGATGTTTTTTAGGGGATTCTACAGCACGACTGAGCAGTGCGTTAGTCGG CAATCTTTGCATACTACGCAGCATTGGGATACAGTGTGAAAAGCAGCA (SEQ ID NO: 2771)). FIG. 72 shows a plot of ADAR1+2% on-target editing (x-axis) versus ADAR1-only % on-target editing (y-axis). As shown in this figure, several gRNAs of the present disclosure (structures of the guide-target RNA scaffold shown in FIG. 73), that comprise an A-G mismatch, show an ADAR2 preference. Thus, an A-G mismatch at the target A may potentially drive ADAR2-specific editing.

### Example 23

### Engineering of LRRK2 Guide RNAs Selected Using HTS

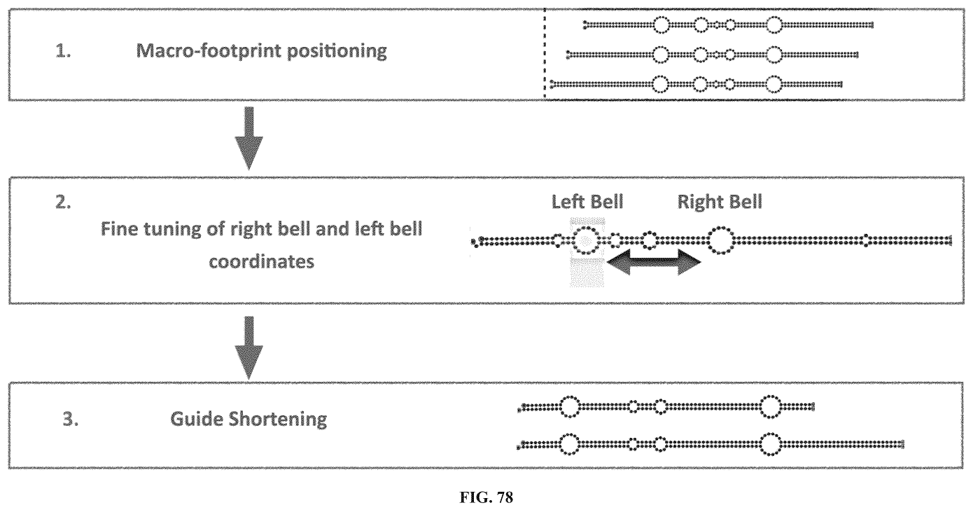

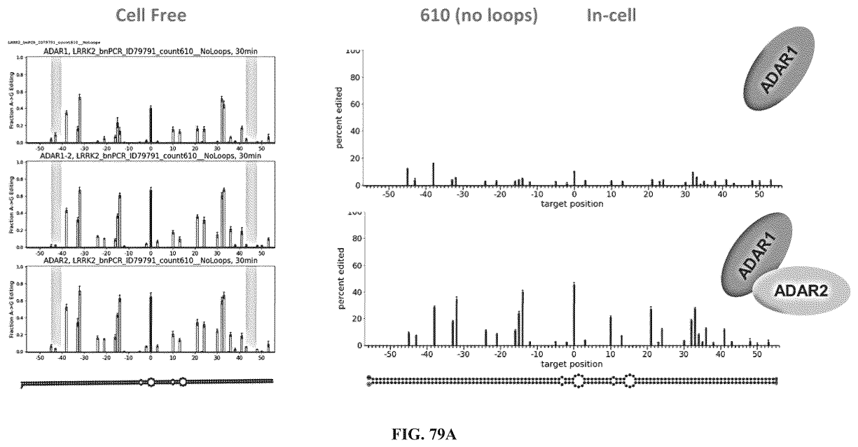

This example describes engineering of guide RNAs using a high throughput screen (see EXAMPLE 14) for targeting of the LRRK2 G2019S mutation for RNA editing by ADAR. FIG. 78 provides an overview of the engineering process. As depicted in FIG. 78, the engineering process includes: 1. positioning of the macro-footprint; 2. fine-tuning of the left-barbell and right-barbell coordinates; and 3. shortening of the guide length. LRRK2 guide RNAs count610 (−14, 26)—SEQ ID NO: 121, count871 (−16, 24)—SEQ ID NO: 139, and count919 (−12, 24)—SEQ ID NO: 166 were selected for further engineering.

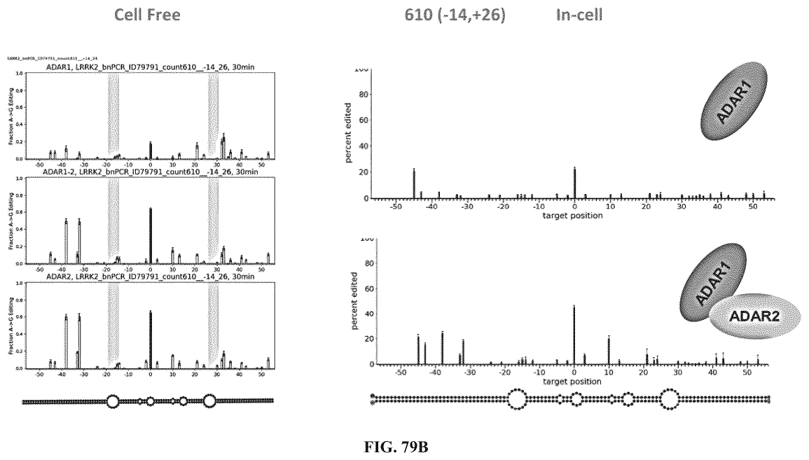

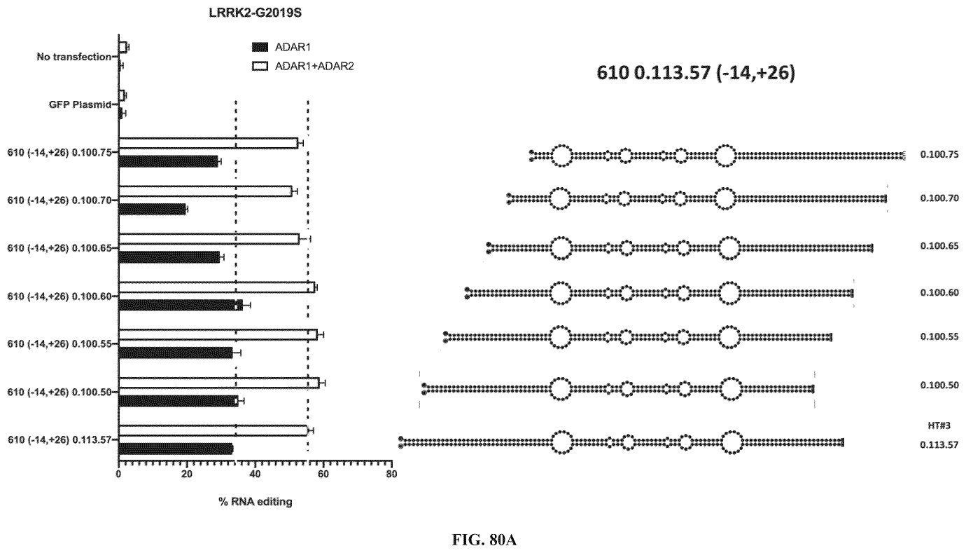

The addition of barbell macro-footprints formed in the guide-target RNA scaffold results in an increase in on-target adenosine editing relative to the amount of off-target editing. As demonstrated in FIG. 79A and FIG. 79B, guide610 (forms a barbell macro-footprint upon hybridization to target RNA with barbells at position −14, +26) displayed a reduction in off-target editing for both ADAR1 and ADAR1+ADAR2, relative to the same engineered guide RNAs lacking the latent structure that would result in a a barbell macro-footprint upon hybridization to target RNA.

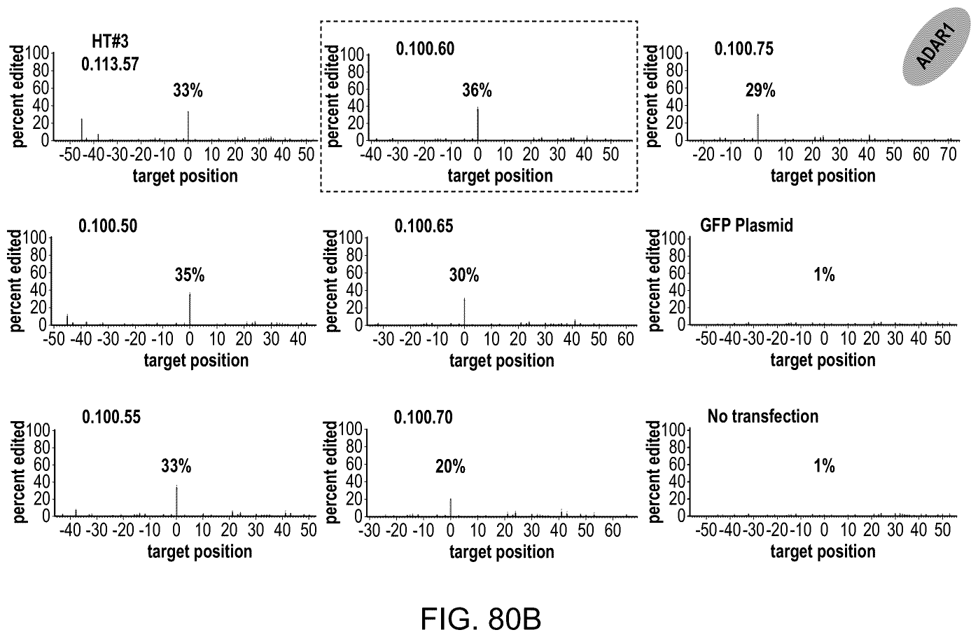

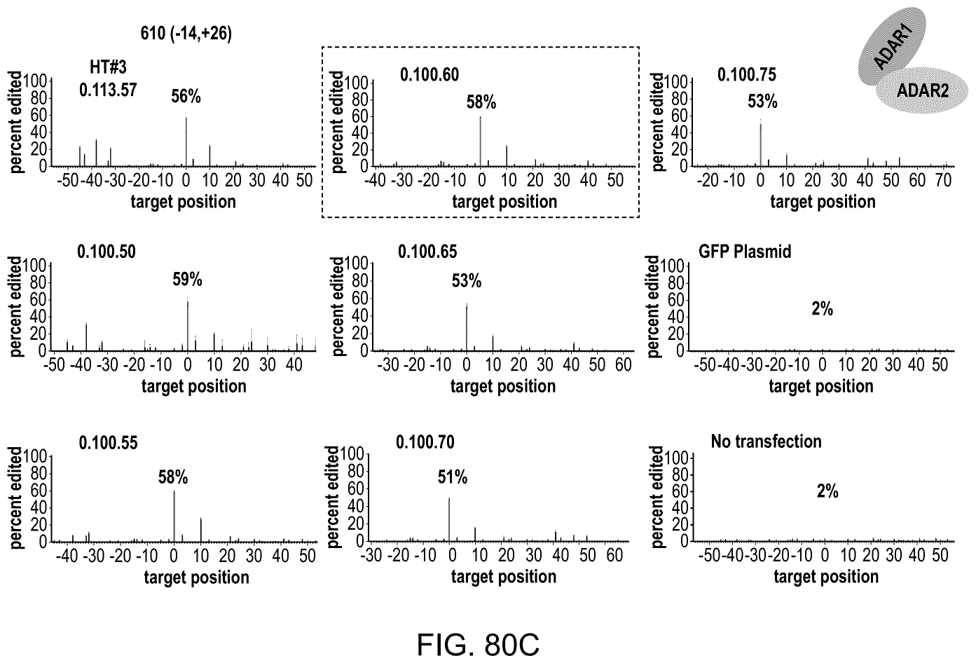

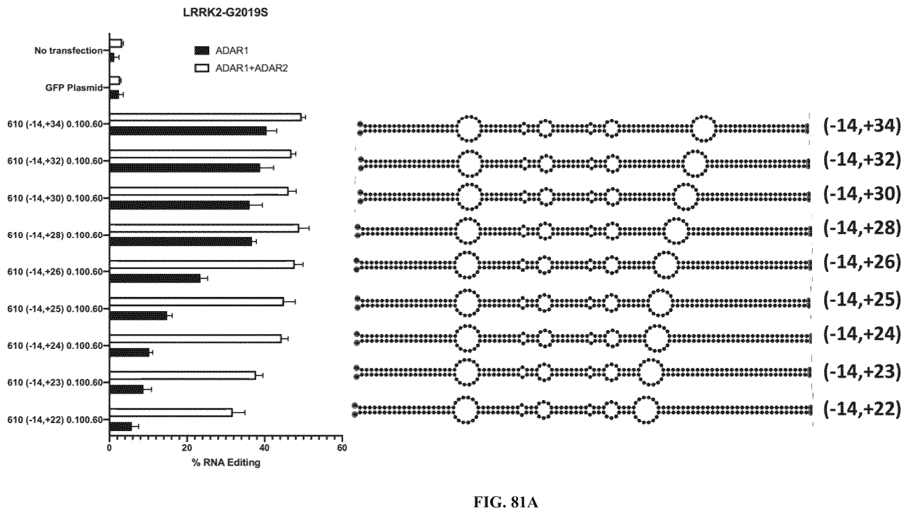

FIG. 80A-FIG. 80C depict the first step of the design process for guide 610: positioning of the macro-footprint. FIG. 80A shows tiling of the macro-footprint positioning within the guide-target RNA scaffold for guide610 with respect to the A/C mismatch and how this tiling affects RNA editing by ADAR1 and ADAR1+ADAR2. FIG. 80B shows the percent editing for the tiled guide610 variants via ADAR1. As noted, the engineered guide RNA with the mismatch positioned 60 nucleotides (0.100.60) from the end of the guide, displaying a LRRK2 editing of 36%, was selected for further engineering. FIG. 80C shows the percent editing for the tiled guide610 variants via ADAR1+ADAR2. As noted, the engineered guide RNA with the mismatch positioned 60 nucleotides (0.100.60) from the end of the engineered guide RNA, displaying a LRRK2 editing of 58%, was selected for further engineering.

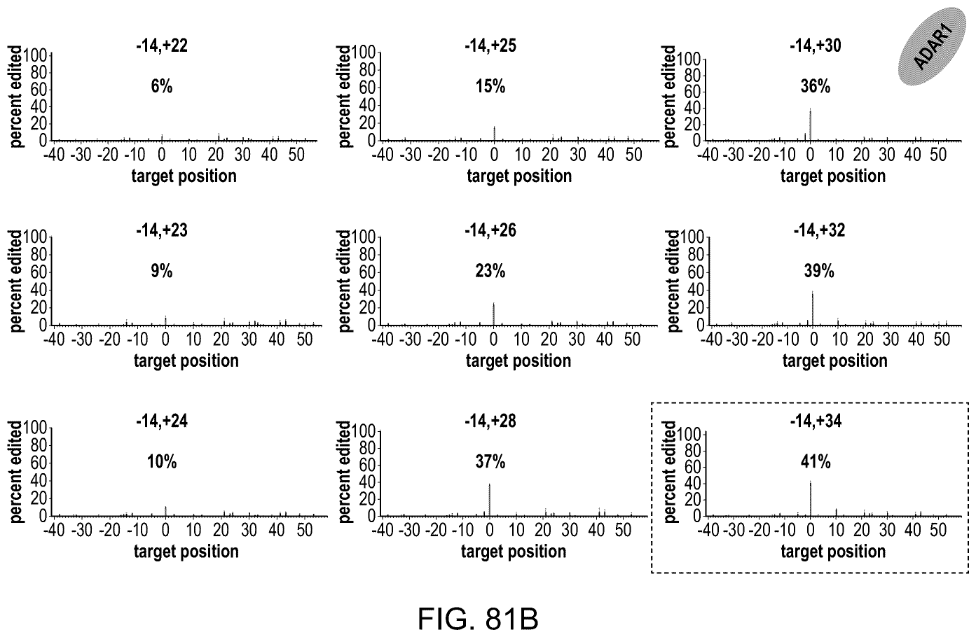

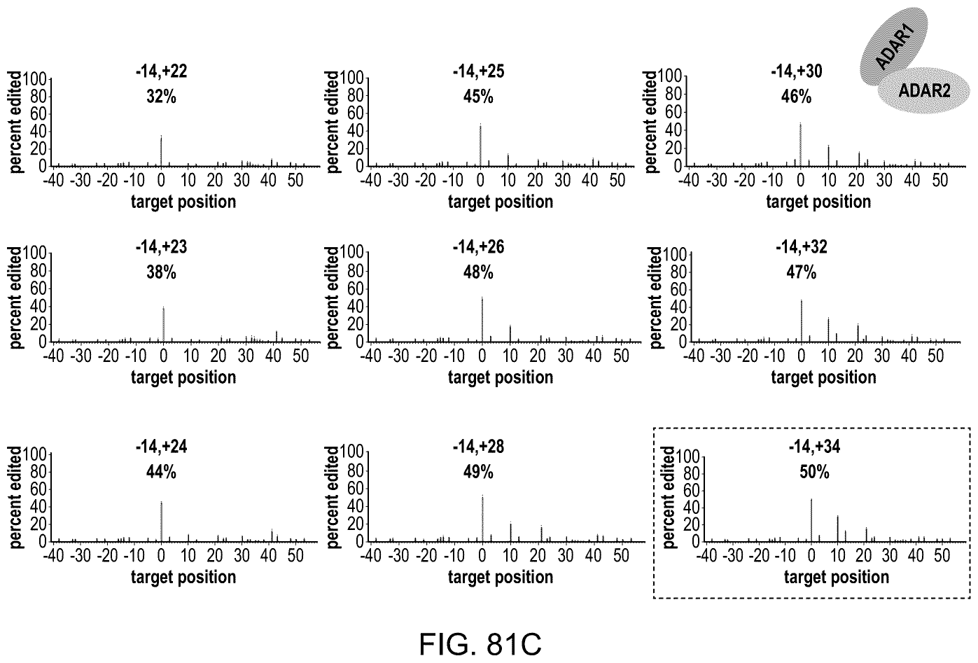

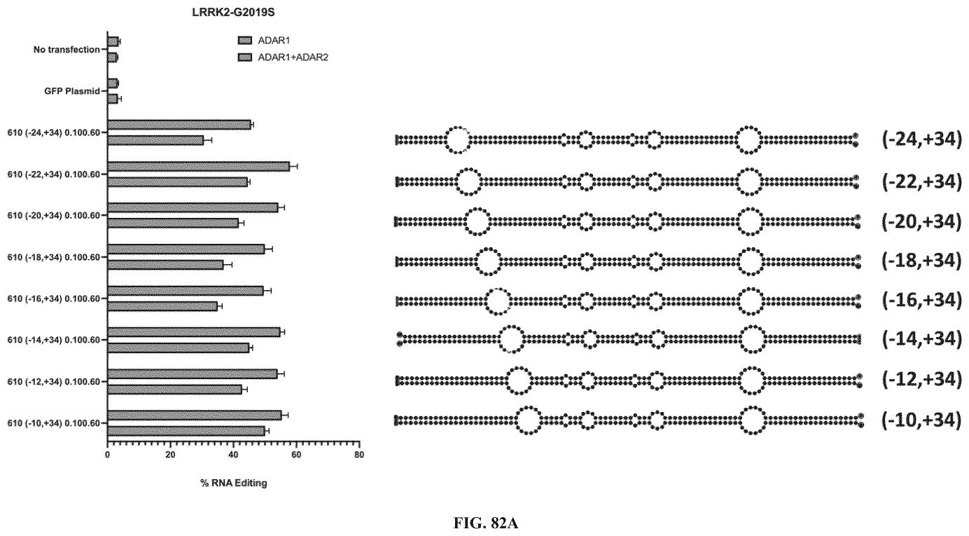

The 0.100.60 guide610 was carried into the next step of design: fine-tuning of the left-barbell and right-barbell coordinates. FIG. 81A-FIG. 81C show engineering of the right barbell coordinates. As shown in FIG. 81A, the coordinate of the right barbell was tiled between the following coordinates with respect to the A/C mismatch: +22, +23, +24, +25, +26, +28, +30, +32, and +34, and the effect of each position on ADAR1 and ADAR1+ADAR2 editing was determined. FIG. 81B shows the percent editing for the tiled guide610 variants via ADAR1. As noted, the guide with the right barbell at position +34 (with respect to the A/C mismatch), displaying a LRRK2 editing of 41%, was selected for further engineering. FIG. 81C shows the percent editing for the tiled guide610 variants via ADAR1+ADAR2. As noted, the guide with the right barbell at position +34 (with respect to the A/C mismatch), displaying a LRRK2 editing of 50% via ADAR, was selected for further engineering.

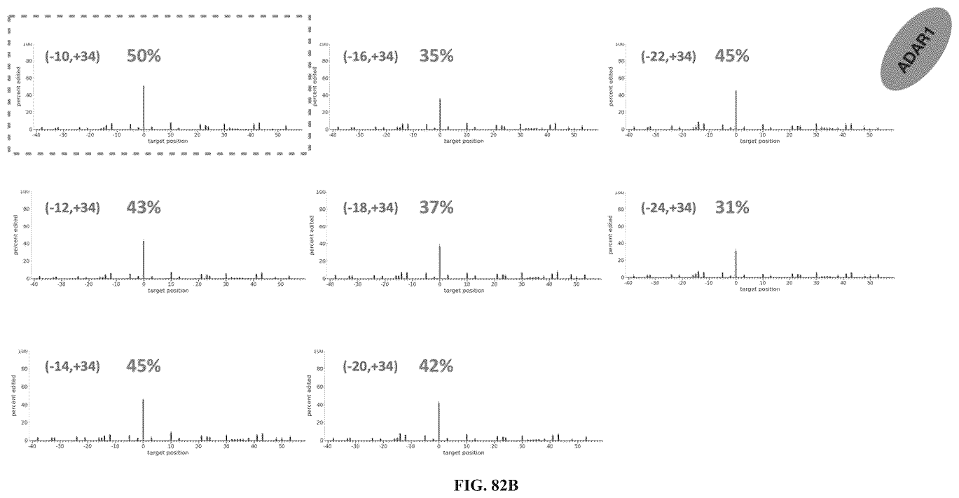

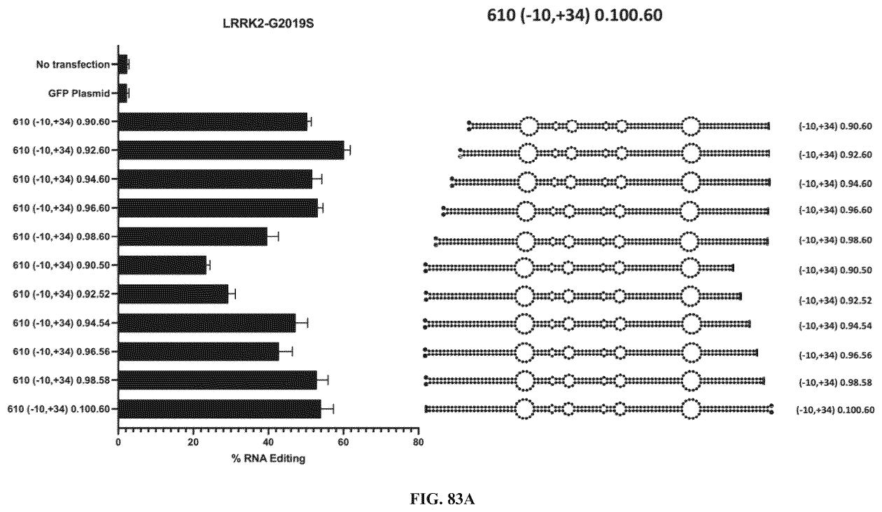

The 0.100.60 guide610 having a right barbell at position +34 (with respect to the A/C mismatch) was utilized as a starting scaffold for left-barbell coordinate tiling. FIG. 82A and FIG. 82B show engineering of the left barbell coordinates. As shown in FIG. 82A, the coordinate of the left barbell was tiled between the following coordinates with respect to the A/C mismatch: −10, −12, −14, −16, −18, −20, −22, and −24, and the effect of each position on ADAR1 and ADAR1+ADAR2 editing was determined. FIG. 82B shows the percent editing for the tiled guide610 variants via ADAR1. As noted, the guide with the left barbell at position −10 and right barbell at position +34 (with respect to the A/C mismatch), displaying a LRRK2 editing of 50% via ADAR, was selected for further engineering.

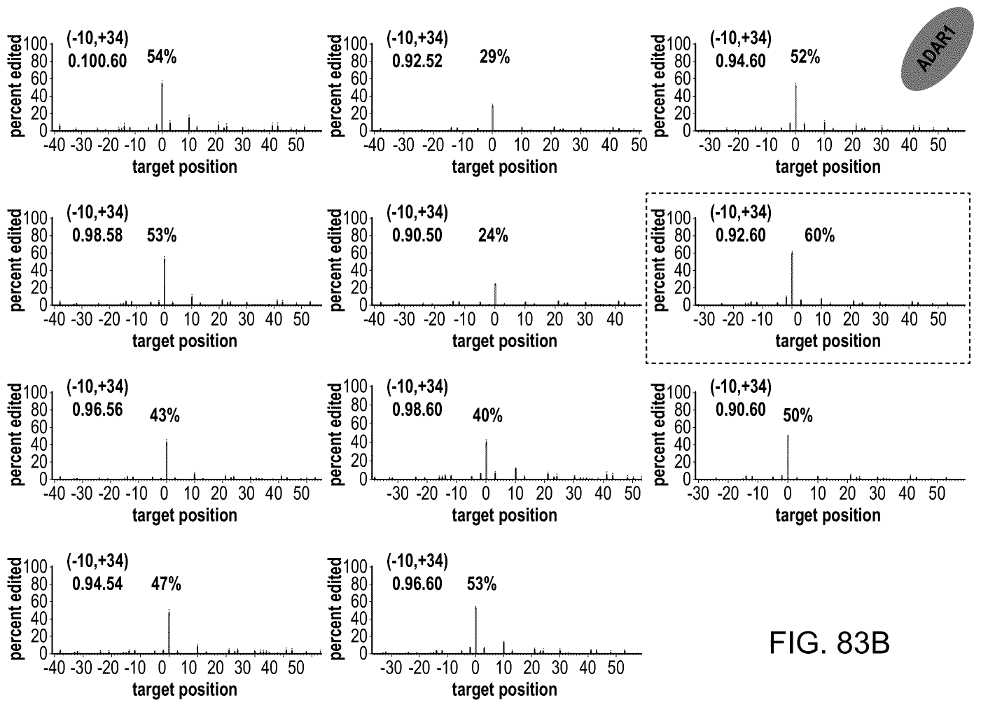

The guide610 variant having barbell coordinates at (−10, +34) was then subjected to the third stage of design: shortening of the guide length. FIG. 83A and FIG. 83B show engineering of the guide length. As shown in FIG. 83A, the effect of each guide length on ADAR1 and ADAR1+ADAR2 editing was determined. FIG. 83B shows the percent editing for the guide610 variants of varying length via ADAR1. As noted, the engineered guide RNA having a length of 92 nt with the mismatch positioned 60 nt from the end of the guide (0.92.60), displaying a LRRK2 editing of 60%, was selected as the top performing guide. TABLE 12 below recites the sequences of the engineered guide610 RNAs depicted in FIGS. 79A-83B. While the engineered guide RNA sequences in TABLE 12 are provided as DNA sequences with a T substituted for each U, the corresponding RNA sequences are also encompassed herein.

TABLE 12
Engineered LRRK2 Guide610 Variant Sequences

| SEQ | | | | | Left | Right |
|---|---|---|---|---|---|---|
| ID | | | | | Barbell | Barbell |
| NO | FIG. | Guide Name | Sequence | Structural Features (target/guide) | Position | Position |
| 120 | 79A | 610 (no loops) | CCCTGGTGTGCCCTCTGATGTTTTT | −49_1-1_wobble_U-G, −4_1- | | |
| | | | ATCCCCATTCTACATCTGTAGTGA | 1_mismatch_C-C, −3_1-1_wobble_U-G, | | |
| | | | GCAATTCCGTGCTCAGCAATCTTT | 0_1-1_mismatch_A-C, 2_1- | | |
| | | | GCAATGATGGCAGCATTGGGATA | 1_mismatch_C-U, 11_1-1_mismatch_G- | | |
| | | | CAGTGTGAAGAGCAGCA | G, 15_1-1_mismatch_U-U, 17_1- | | |
| | | | | 1_mismatch_C-U | | |
| 121 | 79B | 610 (−14, +26) | CCCTGGTGTGCCCTCTGATGTTTTT | −49_1-1_wobble_U-G, −14_6- | −14 | 26 |
| | | | TAGGGGATTCTACATCTGTAGTGA | 6_internal_loop-symmetric_UGCAAA- | | |
| | | | GCAATTCCGTGCTCAGCAATCAAA | AAACGU, −4_1-1_mismatch_C-C, | | |
| | | | CGTATGATGGCAGCATTGGGATAC | −3_1-1_wobble_U-G, 0_1- | | |
| | | | AGTGTGAAGAGCAGCA | 1_mismatch_A-C, 2_1-1_mismatch_C- | | |
| | | | | U, 11_1-1_mismatch_G-G, 15_1- | | |
| | | | | 1_mismatch_U-U, 17_1-1_mismatch_C- | | |
| | | | | U, 26_6-6_internal_loop- | | |
| | | | | symmetric_GGGGAU-UAGGGG | | |
| 2896 | 80A-80C | 610 (−14, +26) | AACTTCAGGTGCACGAAACCCTG | −14_6-6_internal_loop- | −14 | 26 |
| | | 0.100.75 | GTGTGCCCTCTGATGTTTTTTAGG | symmetric_UGCAAA-AAACGU, −4_1- | | |
| | | | GGATTCTACATCTGTAGTGAGCAA | 1_mismatch_C-C, −3_1-1_wobble_U-G, | | |
| | | | TTCCGTGCTCAGCAATCAAACGTA | 0_1-1_mismatch_A-C, 2_1- | | |
| | | | TGATG | 1_mismatch_C-U, 11_1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 2897 | 80A-80C | 610 (−14, +26) | CAGGTGCACGAAACCCTGGTGTG | −14_6-6_internal_loop- | −14 | 26 |
| | | 0.100.70 | CCCTCTGATGTTTTTTAGGGGATT | symmetric_UGCAAA-AAACGU, −4_1- | | |
| | | | CTACATCTGTAGTGAGCAATTCCG | 1_mismatch_C-C, −3_1-1_wobble_U-G, | | |
| | | | TGCTCAGCAATCAAACGTATGATG | 0_1-1_mismatch_A-C, 2_1- | | |
| | | | GCAGC | 1_mismatch_C-U, 11_1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 288 | 80A-80C | 610 (−14, +26) | GCACGAAACCCTGGTGTGCCCTCT | −14_6-6_internal_loop- | −14 | 26 |
| | | 0.100.65 | GATGTTTTTTAGGGGATTCTACAT | symmetric_UGCAAA-AAACGU, −4_1- | | |
| | | | CTGTAGTGAGCAATTCCGTGCTCA | 1_mismatch_C-C, −3_1-1_wobble_U-G, | | |
| | | | GCAATCAAACGTATGATGGCAGC | 0_1-1_mismatch_A-C, 2_1- | | |
| | | | ATTGG | 1_mismatch_C-U, 11_1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 2899 | 80A-80C | 610 (−14, +26) | AAACCCTGGTGTGCCCTCTGATGT | −14_6-6_internal_loop- | −14 | 26 |
| | | 0.100.60 | TTTTTAGGGGATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 2900 | 80A-80C | 610 (−14, +26) | CTGGTGTGCCCTCTGATGTTTTTTA | −4_6-6_internal_loop- | −14 | 26 |
| | | 0.100.55 | GGGGATTCTACATCTGTAGTGAGC | symmetric_UGCAAA-AAACGU, −4_1- | | |
| | | | AATTCCGTGCTCAGCAATCAAACG | 1_mismatch_C-C, −3_1-1_wobble_U-G, | | |
| | | | TATGATGGCAGCATTGGGATACA | 0_1-1_mismatch_A-C, 2_1- | | |
| | | | GTGT | 1_mismatch_C-U, 11_1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 2901 | 80A-80C | 610 (−14, +26) | GTGCCCTCTGATGTTTTTTAGGGG | −14_6-6_internal_loop- | −14 | 26 |
| | | 0.100.50 | ATTCTACATCTGTAGTGAGCAATT | symmetric_UGCAAA-AAACGU, −4_1- | | |
| | | | CCGTGCTCAGCAATCAAACGTATG | 1_mismatch_C-C, −3_1-1_wobble_U-G, | | |
| | | | ATGGCAGCATTGGGATACAGTGT | 0_1-1_mismatch_A-C, 2_1- | | |
| | | | GAAAA | 1_mismatch_C-U, 11_1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 121 | 80A-80C | 610 (−14, +26) | CCCTGGTGTGCCCTCTGATGTTTTT | −49_1-1_wobble_U-G, −14_6- | −14 | 26 |
| | | 0.113.57 | TAGGGGATTCTACATCTGTAGTGA | 6_internal_loop-symmetric_UGCAAA- | | |
| | | | GCAATTCCGTGCTCAGCAATCAAA | AAACGU, −4_1-1_mismatch_C-C, | | |
| | | | CGTATGATGGCAGCATTGGGATAC | −3_1-1_wobble_U-G, 0_1- | | |
| | | | AGTGTGAAGAGCAGCA | 1_mismatch_A-C, 2_1-1_mismatch_C- | | |
| | | | | U, 11_1-1_mismatch_G-G, 15_1- | | |
| | | | | 1_mismatch_U-U, 17_1-1_mismatch_C- | | |
| | | | | U, 26_6-6_internal_loop- | | |
| | | | | symmetric_GGGGAU-UAGGGG | | |
| 2902 | 81A-81C | 610 (−14, +34) | AAACCCTGGTGTGCCCTCTGTCCA | −14_6-6_internal_loop- | −14 | 34 |
| | | 0.100.60 | AATTATCCCCATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2903 | 81A-81C | 610 (−14, +32) | AAACCCTGGTGTGCCCTCTGATCA | −14_6-6_internal_loop- | −14 | 32 |
| | | 0.100.60 | AAACATCCCCATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 32_6- | | |
| | | | | 6_internal_loop-symmetric_AAAAAC- | | |
| | | | | CAAAAC | | |
| 2904 | 81A-81C | 610 (−14, +30) | AAACCCTGGTGTGCCCTCTGATGT | −14_6-6_internal_loop- | −14 | 30 |
| | | 0.100.60 | AAACTACCCCATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 30_6- | | |
| | | | | 6_internal_loop-symmetric_AUAAAA- | | |
| | | | | AAACUA | | |
| 2905 | 81A-81C | 610 (−14, +28) | AAACCCTGGTGTGCCCTCTGATGT | −14_6-6_internal_loop- | −14 | 28 |
| | | 0.100.60 | TTACTAGGCCATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 28_6- | | |
| | | | | 6_internal_loop-symmetric_GGAUAA- | | |
| | | | | ACUAGG | | |
| 2899 | 81A-81C | 610 (−14, +26) | AAACCCTGGTGTGCCCTCTGATGT | −14_6-6_internal_loop- | −14 | 26 |
| | | 0.100.60 | TTTTTAGGGGATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 2906 | 81A-81C | 610 (−14, +25) | AAACCCTGGTGTGCCCTCTGATGT | | −14 | 28 |
| | | 0.100.60 | TTTTAAGGGGTTTCTACATCTGTA | | | |
| | | | GTGAGCAATTCCGTGCTCAGCAAT | | | |
| | | | CAAACGTATGATGGCAGCATTGG | | | |
| | | | GATAC | | | |
| 2907 | 81A-81C | 610 (−14, +24) | AAACCCTGGTGTGCCCTCTGATGT | −14_6-6_internal_loop- | −14 | 24 |
| | | 0.100.60 | TTTTATGGGGTCTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 24_6- | | |
| | | | | 6_internal_loop-symmetric_AUGGGG- | | |
| | | | | GGGGUC | | |
| 2908 | 81A-81C | 610 (−14, +23) | AAACCCTGGTGTGCCCTCTGATGT | −14_6-6_internal_loop- | −14 | 23 |
| | | 0.100.60 | TTTTATCGGGTCACTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 23_6- | | |
| | | | | 6_internal_loop-symmetric_AAUGGG- | | |
| | | | | GGGUCA | | |
| 2909 | 81A-81C | 610 (−14, +22) | AAACCCTGGTGTGCCCTCTGATGT | −14_6-6_internal_loop- | −14 | 22 |
| | | 0.100.60 | TTTTATCCGGTCAGTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 22_6- | | |
| | | | | 6_internal_loop-symmetric_GAAUGG- | | |
| | | | | GGUCAG | | |
| 2910 | 82A-82B | 610 (−24, +34) | AAACCCTGGTGTGCCCTCTGTCCA | −24_6-6_internal_loop- | −24 | 34 |
| | | 0.100.60 | AATTATCCCCATTCTACATCTGTA | symmetric_CUGCCA-ACUGUC, −4_1- | | |
| | | | GTGAGCAATTCCGTGCTCAGCAAT | 1_mismatch_C-C, −3_1-1_wobble_U-G, | | |
| | | | CTTTGCAATGAACTGTCCATTGGG | 0_1-1_mismatch_A-C, 2_1- | | |
| | | | ATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2911 | 82A-82B | 610 (−22, +34) | AAACCCTGGTGTGCCCTCTGTCCA | −22 6-6_internal_loop- | −22 | 34 |
| | | 0.100.60 | AATTATCCCCATTCTACATCTGTA | symmetric_GCCAUC-CUACUG, −4_1- | | |
| | | | GTGAGCAATTCCGTGCTCAGCAAT | 1_mismatch_C-C, −3_1-1_wobble_U-G, | | |
| | | | CTTTGCAATCTACTGAGCATTGGG | 0_1-1_mismatch_A-C, 2_1- | | |
| | | | ATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2912 | 82A-82B | 610 (−20, +34) | AAACCCTGGTGTGCCCTCTGTCCA | −20_6-6_internal_loop- | −20 | 34 |
| | | 0.100.60 | AATTATCCCCATTCTACATCTGTA | symmetric_CAUCAU-CCCUAC, −4_1- | | |
| | | | GTGAGCAATTCCGTGCTCAGCAAT | 1_mismatch_C-C, −3_1-1_wobble_U-G, | | |
| | | | CTTTGCACCCTACGCAGCATTGGG | 0_1-1_mismatch_A-C, 2_1- | | |
| | | | ATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2913 | 82A-82B | 610 (−18, +34) | AAACCCTGGTGTGCCCTCTGTCCA | −18_6-6_internal_loop- | −18 | 34 |
| | | 0.100.60 | AATTATCCCCATTCTACATCTGTA | symmetric_UCAUUG-GUCCCU, −4_1- | | |
| | | | GTGAGCAATTCCGTGCTCAGCAAT | 1_mismatch_C-C, −3_1-1_wobble_U-G, | | |
| | | | CTTTGGTCCCTTGGCAGCATTGGG | 0_1-1_mismatch_A-C, 2_1- | | |
| | | | ATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2914 | 82A-82B | 610 (−16, +34) | AAACCCTGGTGTGCCCTCTGTCCA | −16_6-6_internal_loop- | −16 | 34 |
| | | 0.100.60 | AATTATCCCCATTCTACATCTGTA | symmetric_AUUGCA-ACGUCC, −4_1- | | |
| | | | GTGAGCAATTCCGTGCTCAGCAAT | 1_mismatch_C-C, −3_1-1_wobble_U-G, | | |
| | | | CTTACGTCCGATGGCAGCATTGGG | 0_1-1_mismatch_A-C, 2_1- | | |
| | | | ATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2902 | 82A-82B | 610 (−14, +34) | AAACCCTGGTGTGCCCTCTGTCCA | −14_6-6_internal_loop- | −14 | 34 |
| | | 0.100.60 | AATTATCCCCATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2915 | 82A-82B | 610 (−12, +34) | AAACCCTGGTGTGCCCTCTGTCCA | −12_6-6_internal_loop- | −12 | 34 |
| | | 0.100.60 | AATTATCCCCATTCTACATCTGTA | symmetric_CAAAGA-AGAAAC, −4_1- | | |
| | | | GTGAGCAATTCCGTGCTCAGCAAA | 1_mismatch_C-C, −3_1-1_wobble_U-G, | | |
| | | | GAAACCAATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2916 | 82A-82B | 610 (−10, +34) | AAACCCTGGTGTGCCCTCTGTCCA | −0_6-6_internal_loop- | −10 | 34 |
| | | 0.100.60 | AATTATCCCCATTCTACATCTGTA | symmetric_AAGAUU-UCAGAA, −4_1- | | |
| | | | GTGAGCAATTCCGTGCTCAGCTCA | 1_mismatch_C-C, −3_1-1_wobble_U-G, | | |
| | | | GAATGCAATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2917 | 83A-83B | 610 (−10, +34) | AAACCCTGGTGTGCCCTCTGTCCA | −0_6-6_internal_loop- | −10 | 34 |
| | | 0.90.60 | AATTATCCCCATTCTACATCTGTA | symmetric_AAGAUU-UCAGAA, −4_1- | | |
| | | | GTGAGCAATTCCGTGCTCAGCTCA | 1_mismatch_C-C, -3_1-1_wobble_U-G, | | |
| | | | GAATGCAATGATGGCAGC | 0_1-1_mismatch_A-C, 2_1- | | |
| | | | | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2918 | 83A-83B | 610 (−10, +34) | AAACCCTGGTGTGCCCTCTGTCCA | −10_6-6_internal_loop- | −10 | 34 |
| | | 0.92.60 | AATTATCCCCATTCTACATCTGTA | symmetric_AAGAUU-UCAGAA, −4_1- | | |
| | | | GTGAGCAATTCCGTGCTCAGCTCA | 1_mismatch_C-C, −3_1-1_wobble_U-G, | | |
| | | | GAATGCAATGATGGCAGCAT | 0_1-1_mismatch_A-C, 2_1- | | |
| | | | | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2919 | 83A-83B | 610 (−10, +34) | AAACCCTGGTGTGCCCTCTGTCCA | −10_6-6_internal_loop- | −10 | 34 |
| | | 0.94.60 | AATTATCCCCATTCTACATCTGTA | symmetric_AAGAUU-UCAGAA, −4_1- | | |
| | | | GTGAGCAATTCCGTGCTCAGCTCA | 1_mismatch_C-C, −3_1-1_wobble_U-G, | | |
| | | | GAATGCAATGATGGCAGCATTG | 0_1-1_mismatch_A-C, 2_1- | | |
| | | | | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2920 | 83A-83B | 610 (−10, +34) | AAACCCTGGTGTGCCCTCTGTCCA | −10_6-6_internal_loop- | −10 | 34 |
| | | 0.96.60 | AATTATCCCCATTCTACATCTGTA | symmetric_AAGAUU-UCAGAA, −4_1- | | |
| | | | GTGAGCAATTCCGTGCTCAGCTCA | 1_mismatch_C-C, −3_1-1_wobble_U-G, | | |
| | | | GAATGCAATGATGGCAGCATTGG | 0_1-1_mismatch_A-C, 2_1- | | |
| | | | G | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2921 | 83A-83B | 610 (−10, +34) | AAACCCTGGTGTGCCCTCTGTCCA | −10_6-6_internal_loop- | −10 | 34 |
| | | 0.98.60 | AATTATCCCCATTCTACATCTGTA | symmetric_AAGAUU-UCAGAA, −4_1- | | |
| | | | GTGAGCAATTCCGTGCTCAGCTCA | 1_mismatch_C-C, −3_1-1_wobble_U-G, | | |
| | | | GAATGCAATGATGGCAGCATTGG | 0_1-1_mismatch_A-C, 2_1- | | |
| | | | GAT | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2922 | 83A-83B | 610 (−10, +34) | GTGCCCTCTGTCCAAATTATCCCC | −10_6-6_internal_loop- | −10 | 34 |
| | | 0.90.50 | ATTCTACATCTGTAGTGAGCAATT | symmetric_AAGAUU-UCAGAA, −4_1- | | |
| | | | CCGTGCTCAGCTCAGAATGCAATG | 1_mismatch_C-C, −3_1-1_wobble_U-G, | | |
| | | | ATGGCAGCATTGGGATAC | 0_1-1_mismatch_A-C, 2_1- | | |
| | | | | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2923 | 83A-83B | 610 (−10, +34) | GTGTGCCCTCTGTCCAAATTATCC | −10_6-6_internal_loop- | −10 | 34 |
| | | 0.92.52 | CCATTCTACATCTGTAGTGAGCAA | symmetric_AAGAUU-UCAGAA, −4_1- | | |
| | | | TTCCGTGCTCAGCTCAGAATGCAA | 1_mismatch_C-C, −3_1-1_wobble_U-G, | | |
| | | | TGATGGCAGCATTGGGATAC | 0_1-1_mismatch_A-C, 2_1- | | |
| | | | | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2924 | 83A-83B | 610 (−10, +34) | TGGTGTGCCCTCTGTCCAAATTAT | −10 6-6_internal_loop- | −10 | 34 |
| | | 0.94.54 | CCCCATTCTACATCTGTAGTGAGC | symmetric_AAGAUU-UCAGAA, −4_1- | | |
| | | | AATTCCGTGCTCAGCTCAGAATGC | 1_mismatch_C-C, −3_1-1_wobble_U-G, | | |
| | | | AATGATGGCAGCATTGGGATAC | 0_1-1_mismatch_A-C, 2_1- | | |
| | | | | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2925 | 83A-83B | 610 (−10, +34) | CCTGGTGTGCCCTCTGTCCAAATT | −10_6-6_internal_loop- | −10 | 34 |
| | | 0.96.56 | ATCCCCATTCTACATCTGTAGTGA | symmetric_AAGAUU-UCAGAA, −4_1- | | |
| | | | GCAATTCCGTGCTCAGCTCAGAAT | 1_mismatch_C-C, −3_1-1_wobble_U-G, | | |
| | | | GCAATGATGGCAGCATTGGGATA | 0_1-1_mismatch_A-C, 2_1- | | |
| | | | C | 1_mismatch_C-U, 11 1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2926 | 83A-83B | 610 (−10, +34) | ACCCTGGTGTGCCCTCTGTCCAAA | −10_6-6_internal_loop- | −10 | 34 |
| | | 0.98.58 | TTATCCCCATTCTACATCTGTAGT | symmetric_AAGAUU-UCAGAA, −4_1- | | |
| | | | GAGCAATTCCGTGCTCAGCTCAGA | 1_mismatch_C-C, −3_1-1_wobble_U-G, | | |
| | | | ATGCAATGATGGCAGCATTGGGA | 0_1-1_mismatch_A-C, 2_1- | | |
| | | | TAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2916 | 83A-83B | 610 (−10, +34) | AAACCCTGGTGTGCCCTCTGTCCA | −10_6-6_internal_loop- | −10 | 34 |
| | | 0.100.60 | AATTATCCCCATTCTACATCTGTA | symmetric_AAGAUU-UCAGAA, −4_1- | | |
| | | | GTGAGCAATTCCGTGCTCAGCTCA | 1_mismatch_C-C, −3_1-1_wobble_U-G, | | |
| | | | GAATGCAATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |

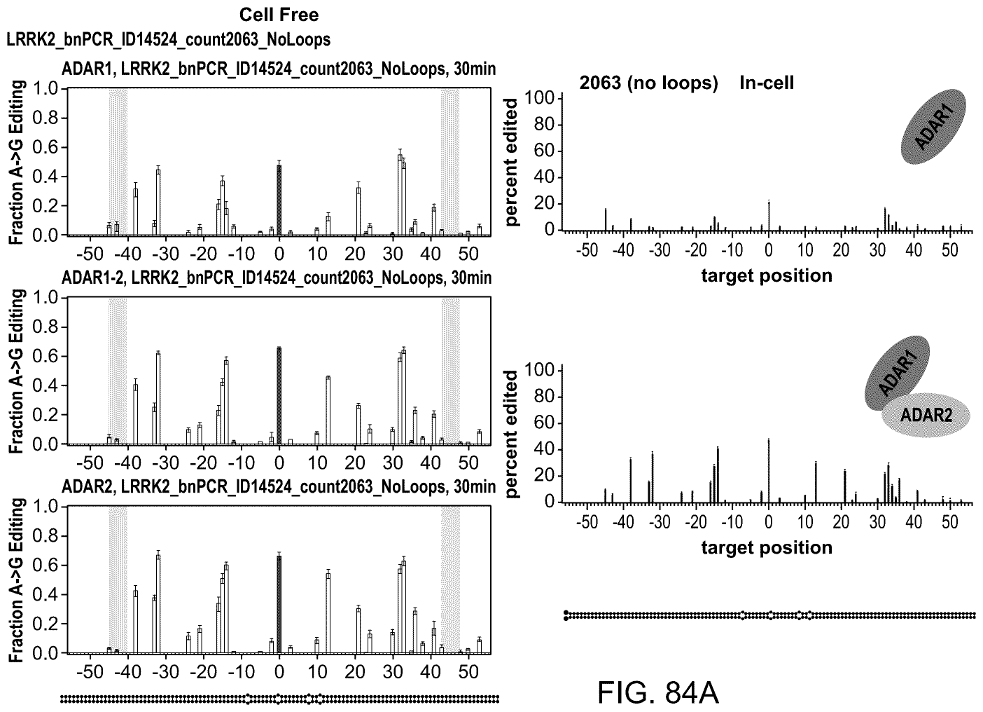

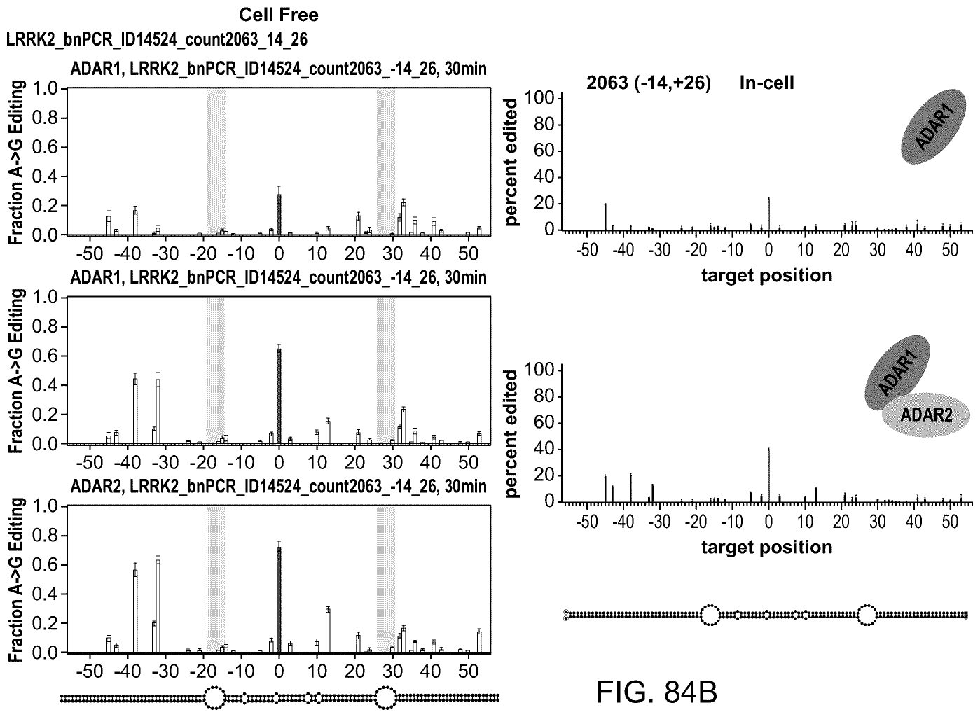

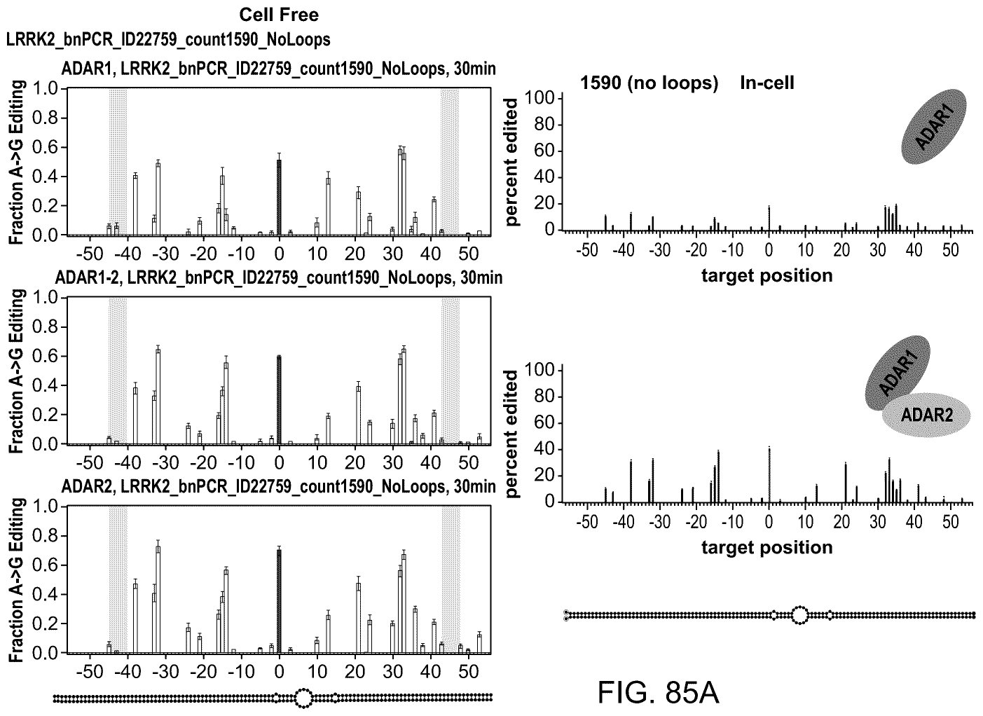

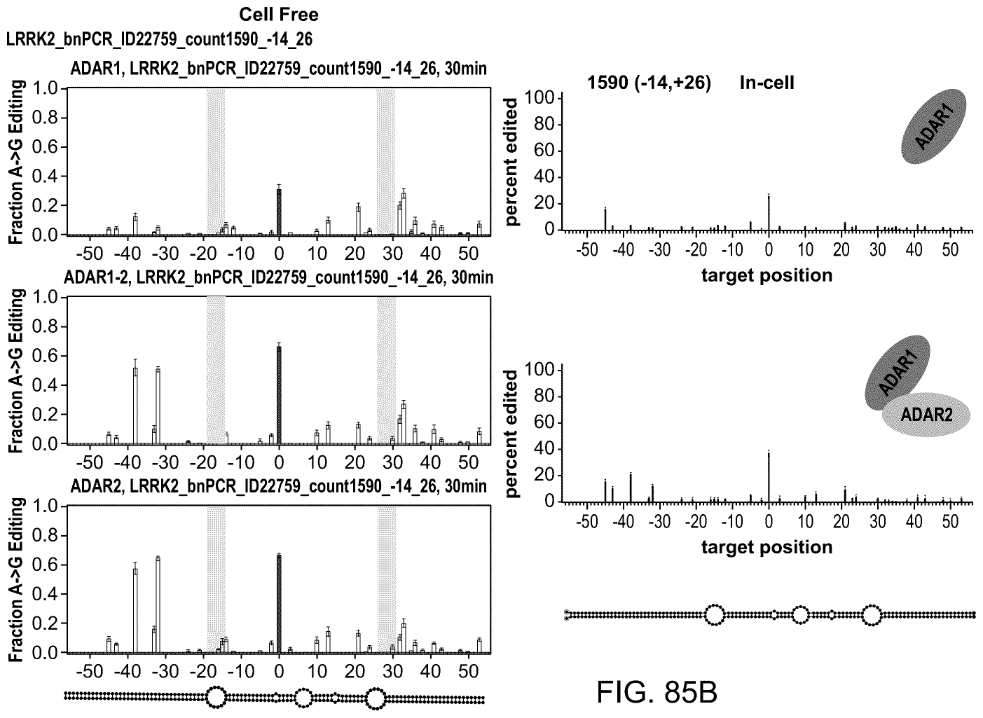

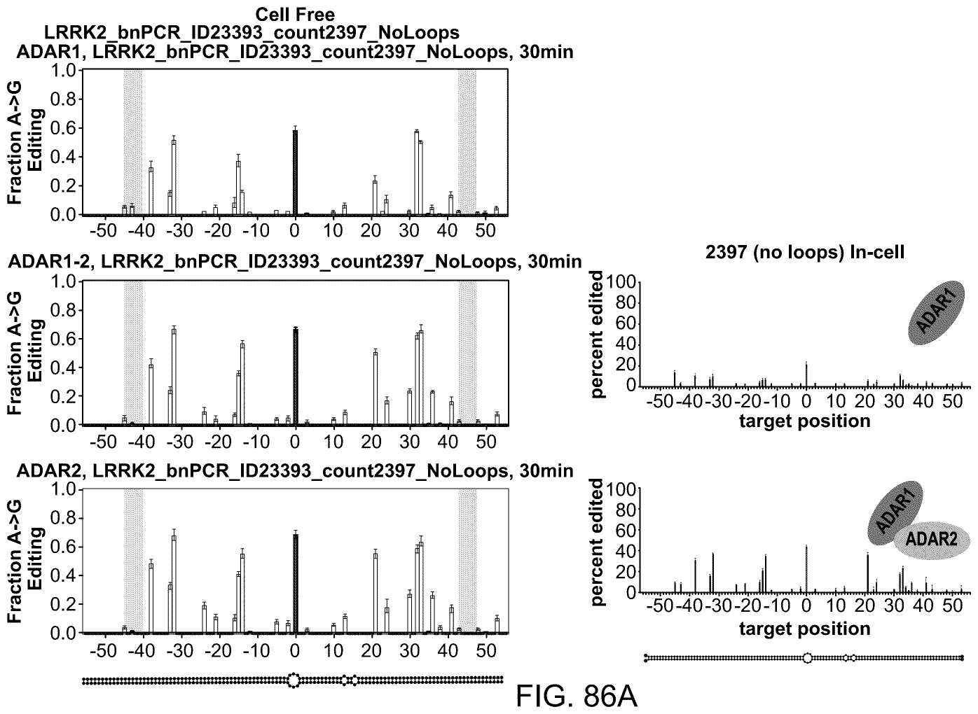

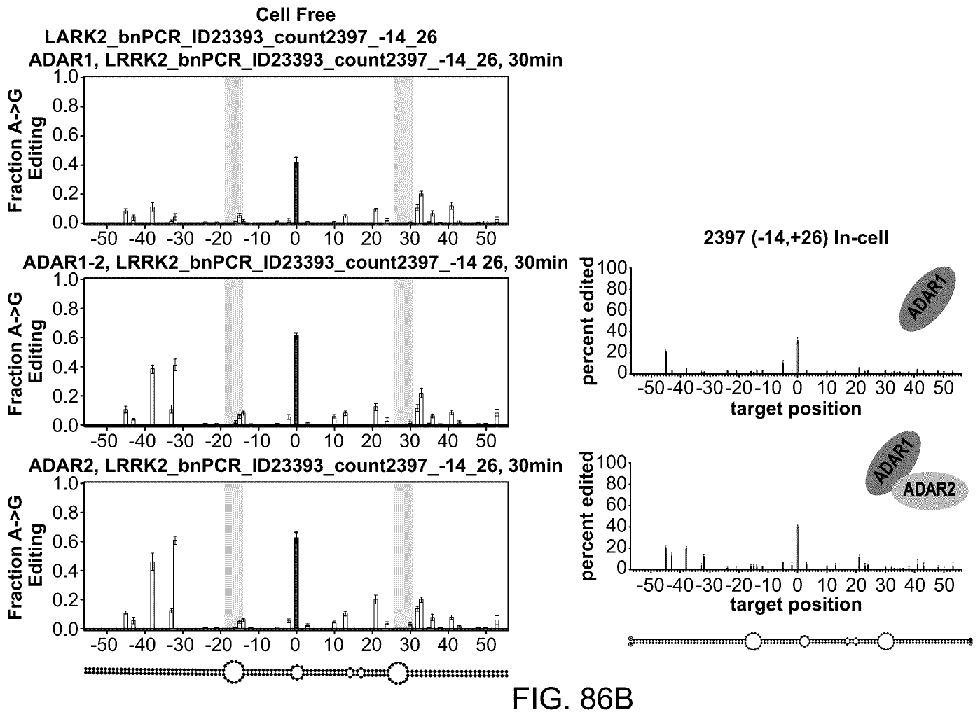

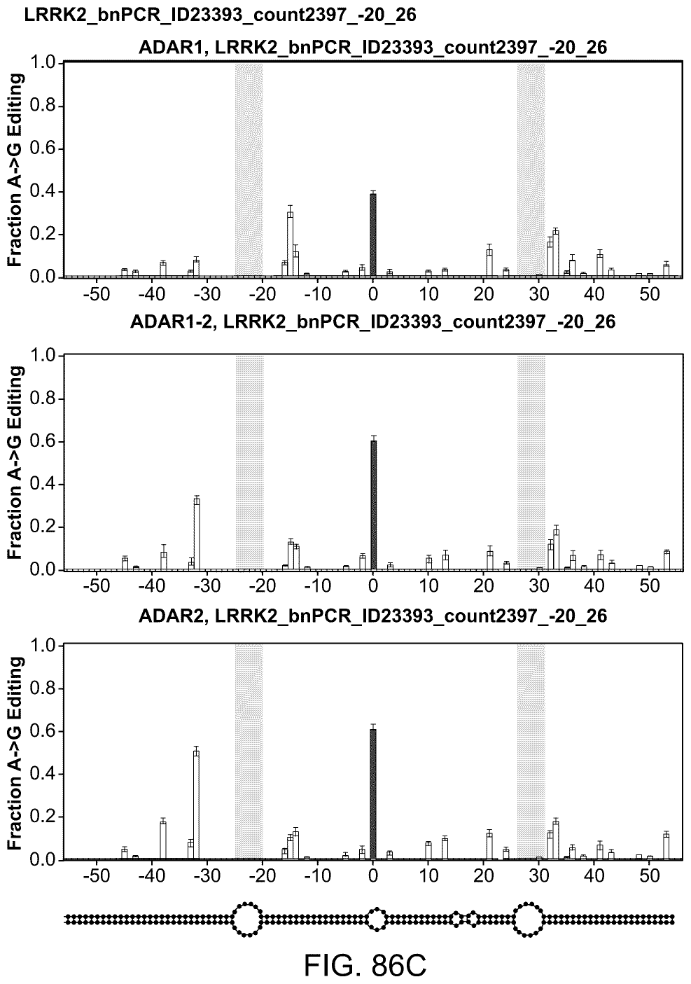

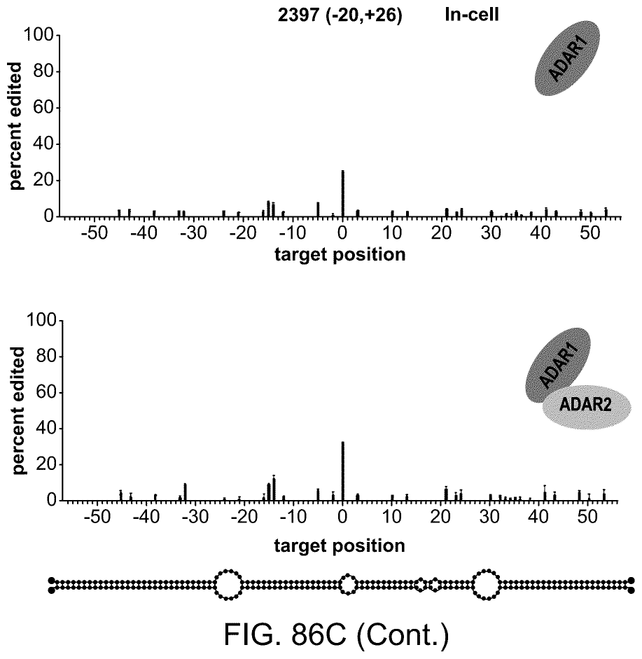

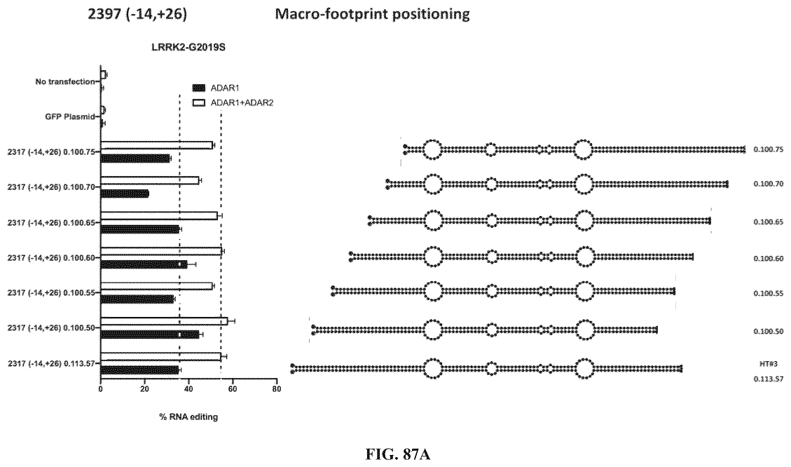

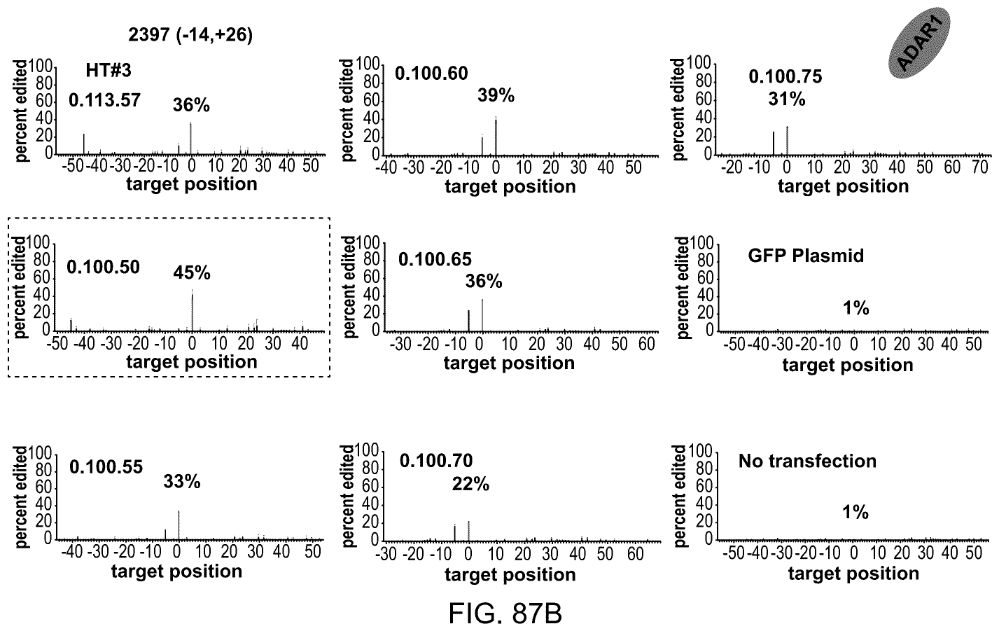

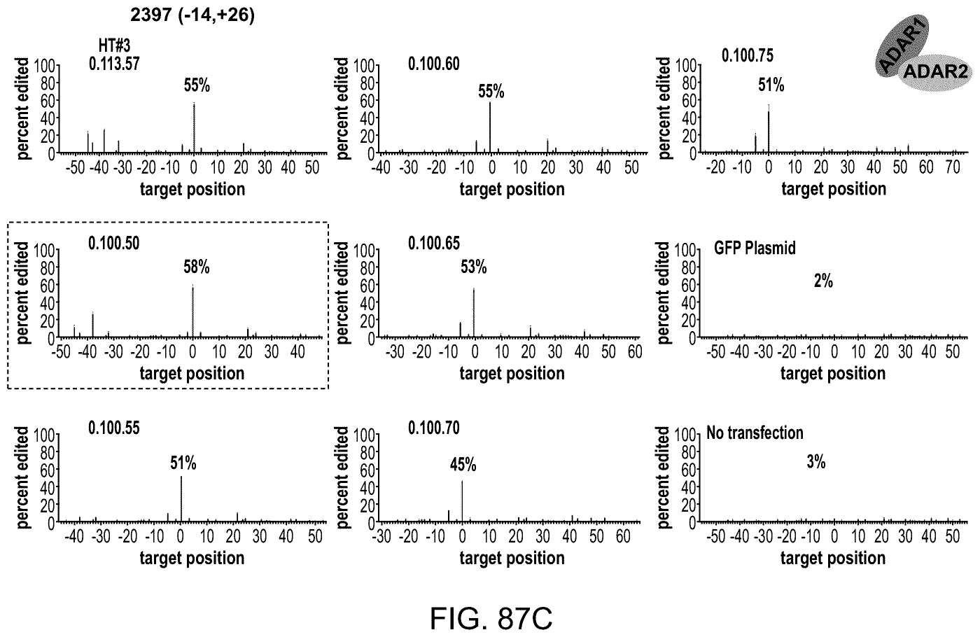

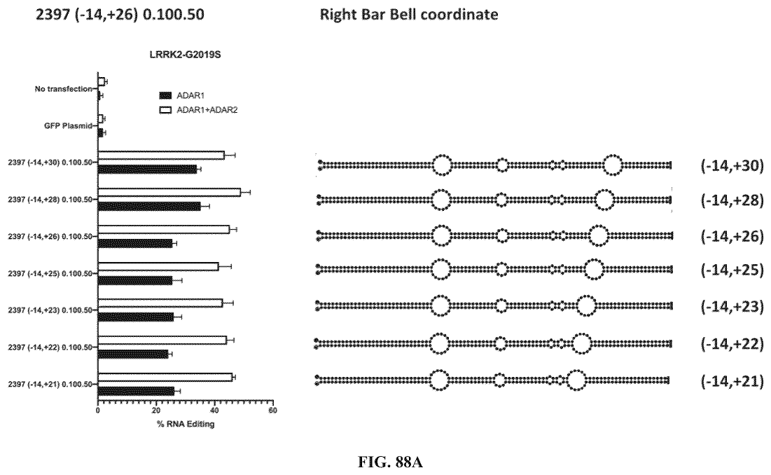

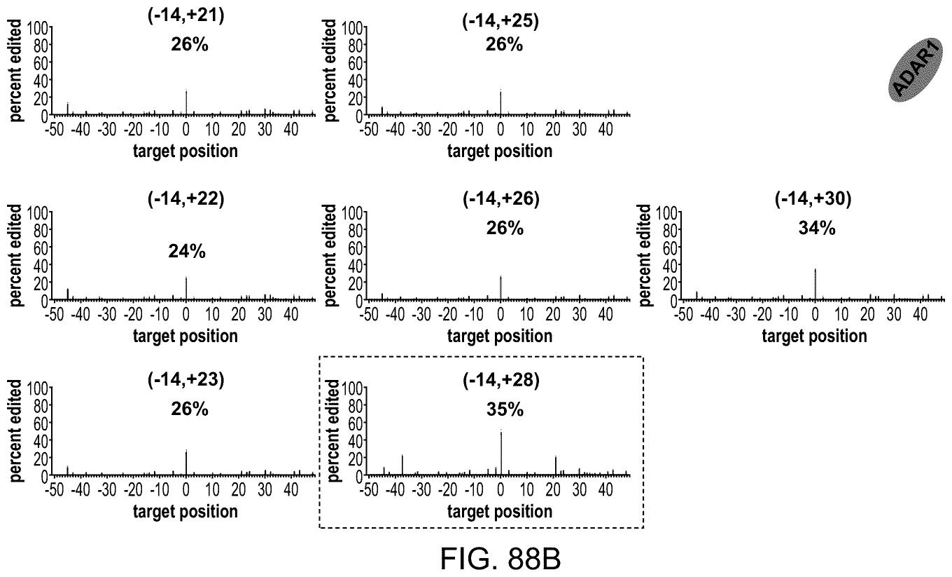

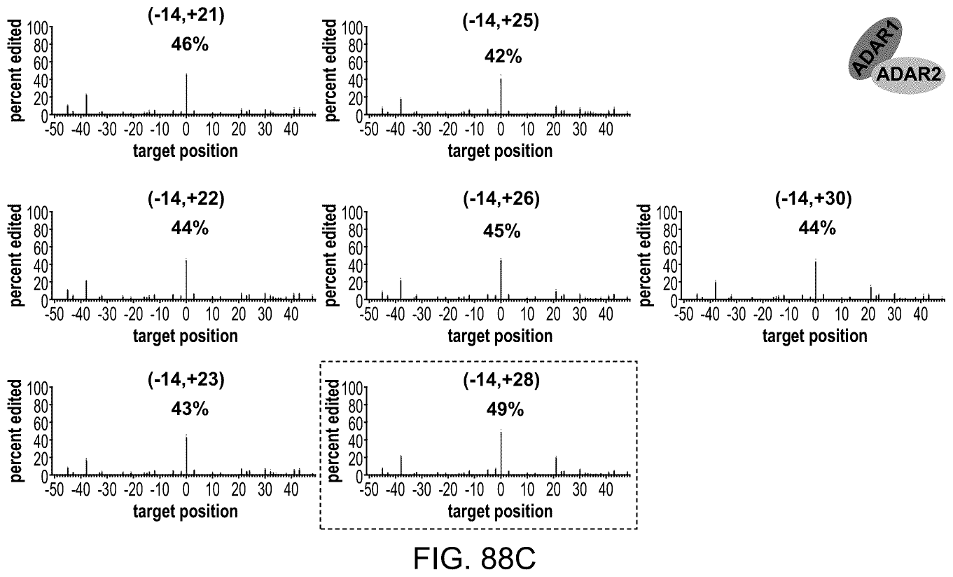

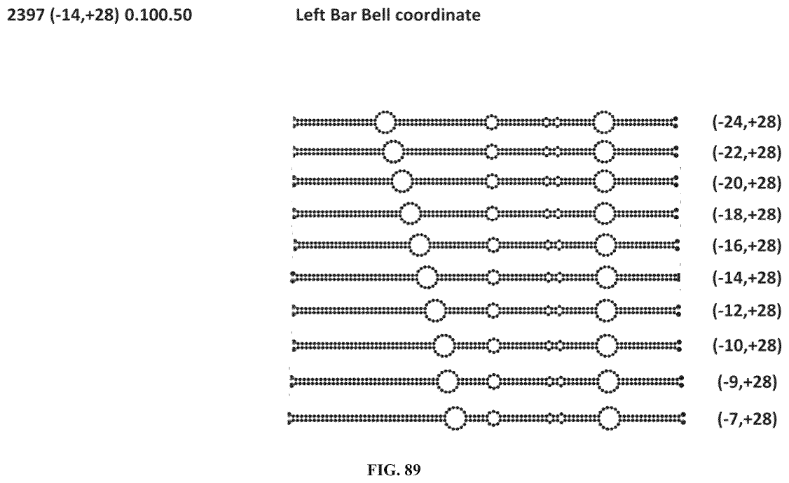

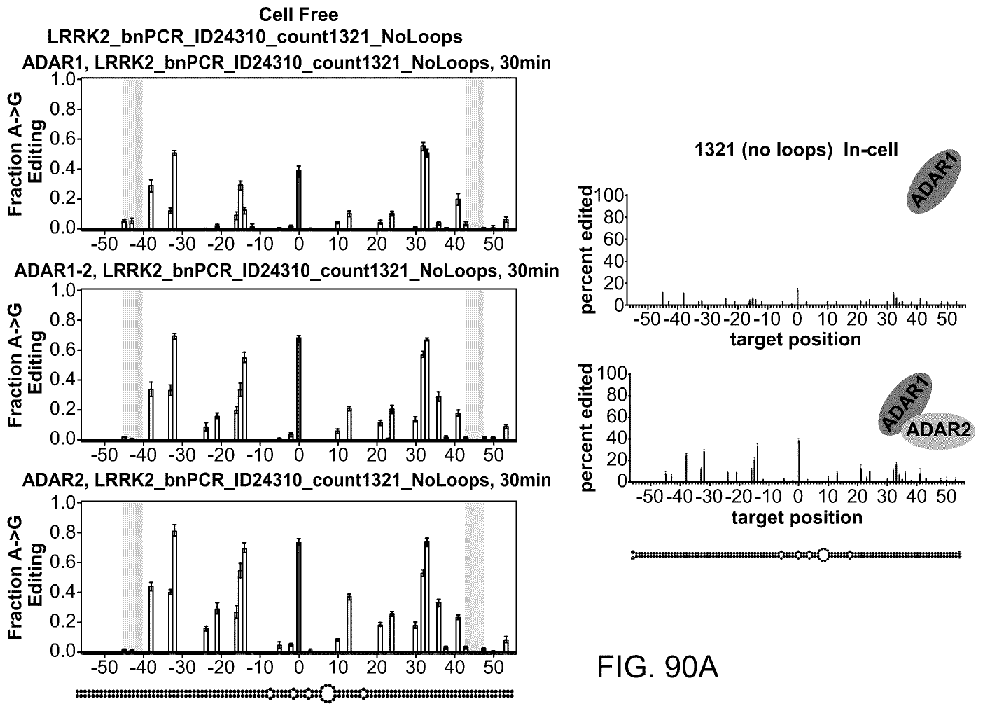

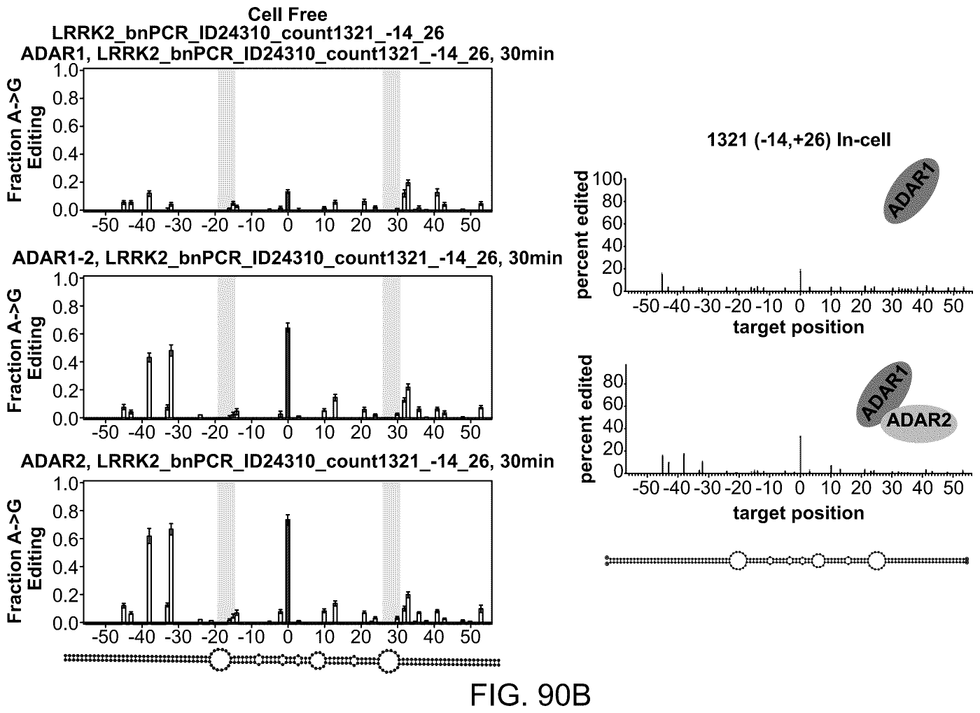

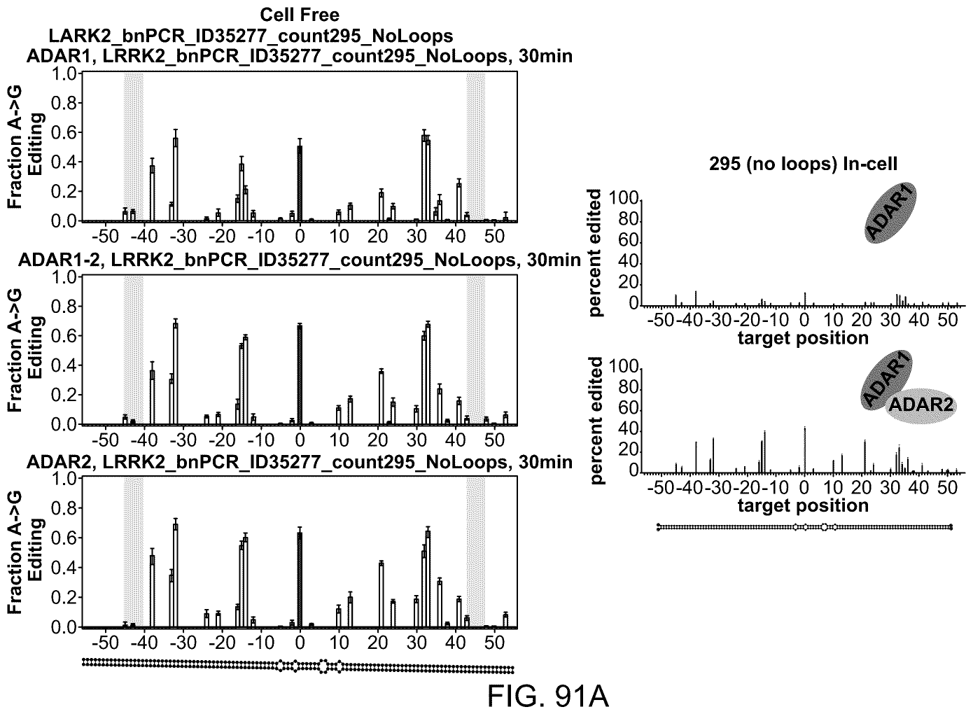

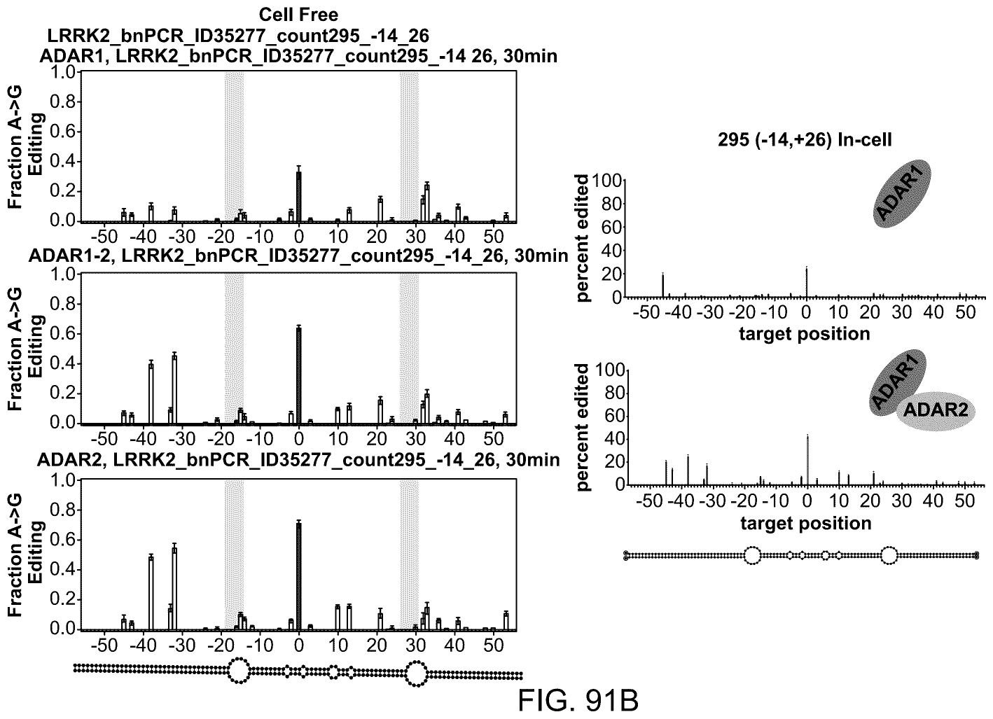

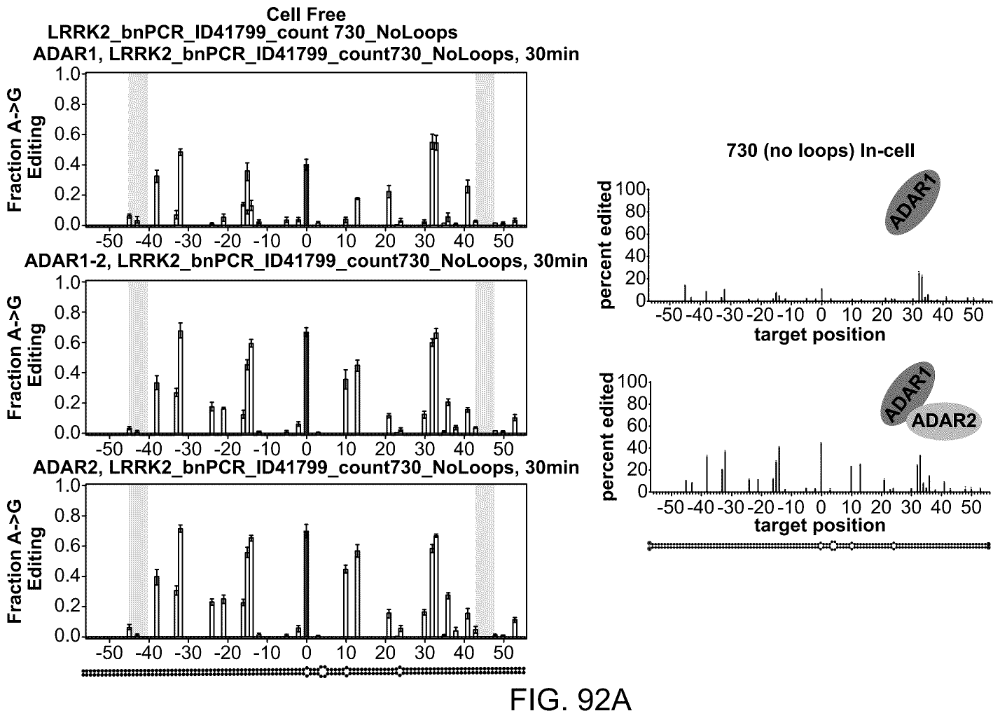

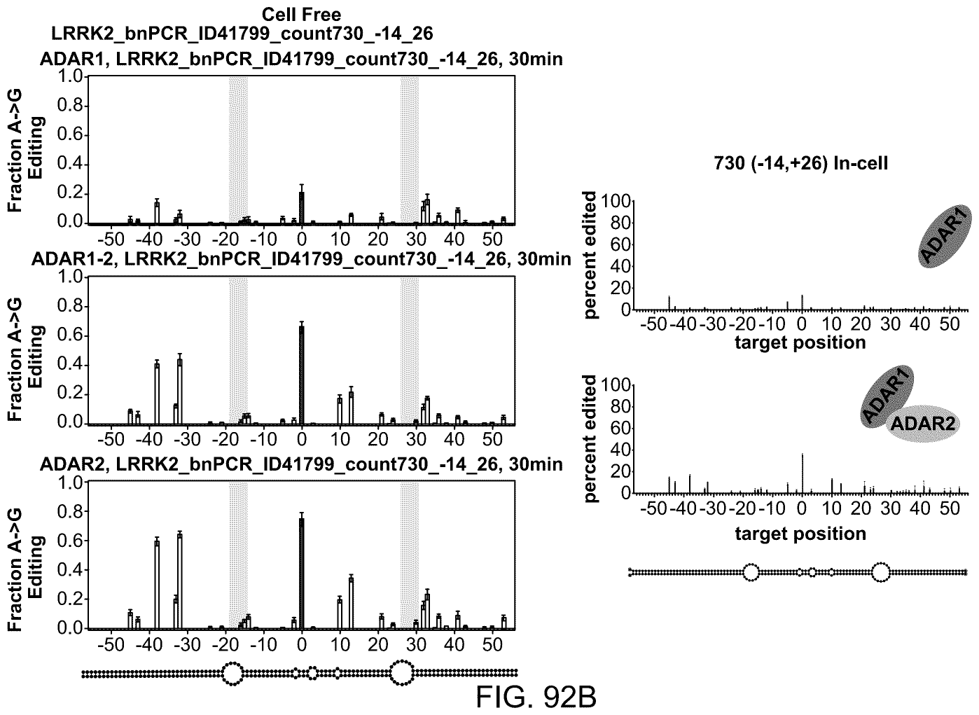

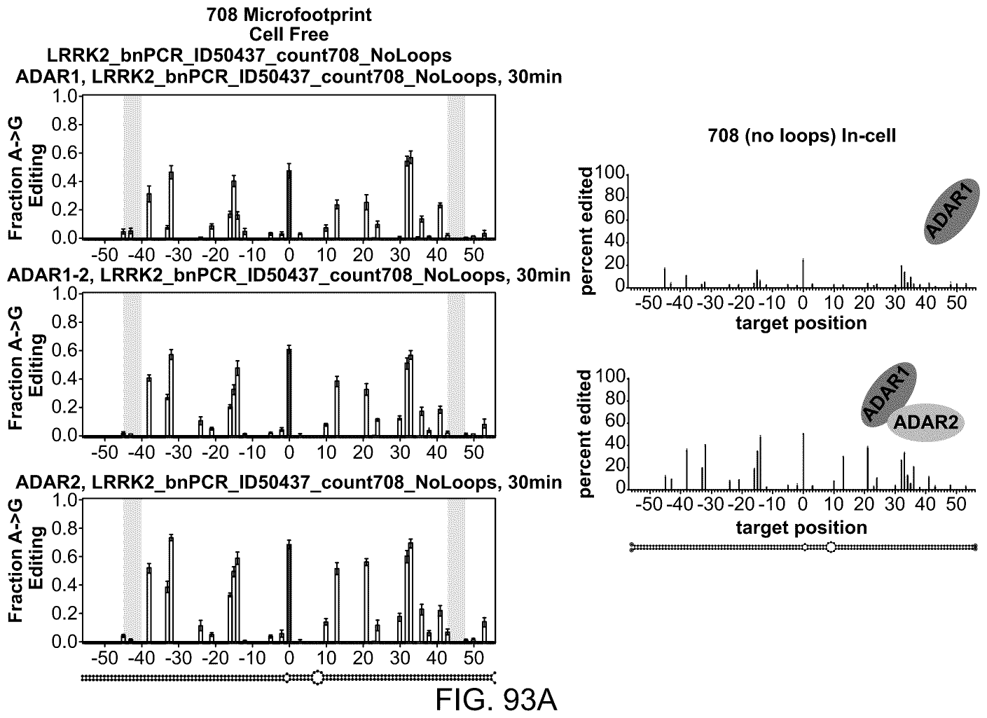

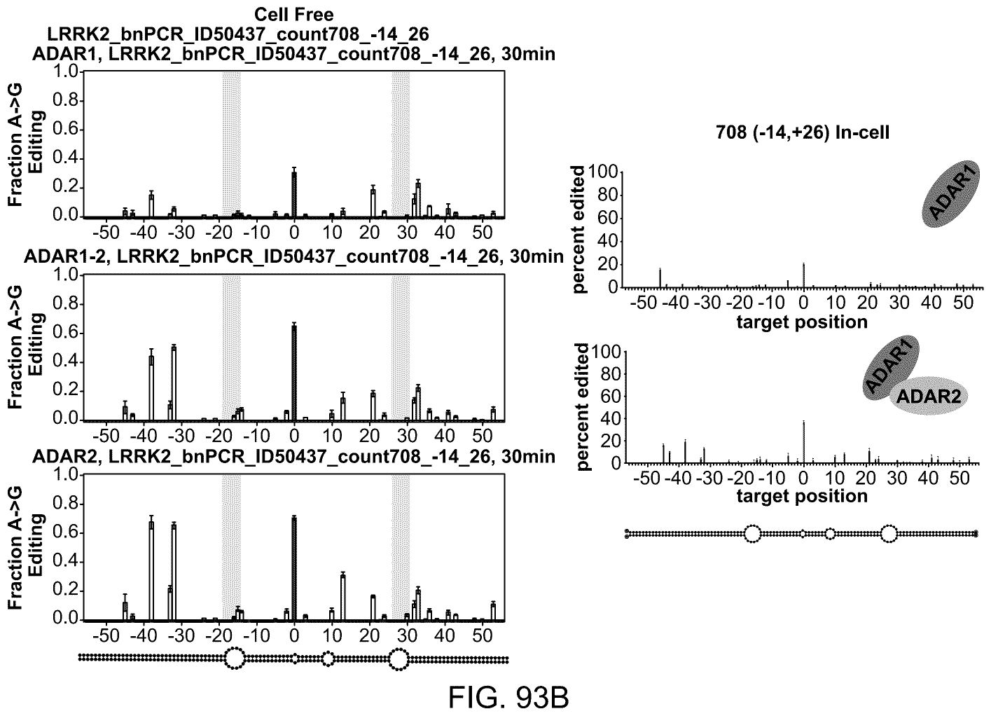

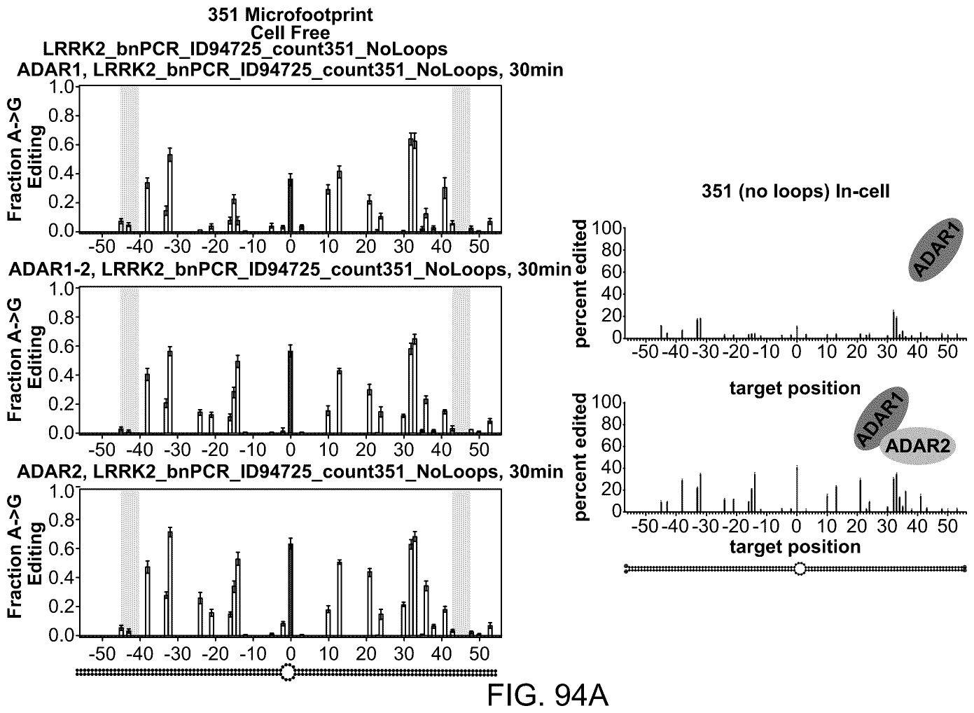

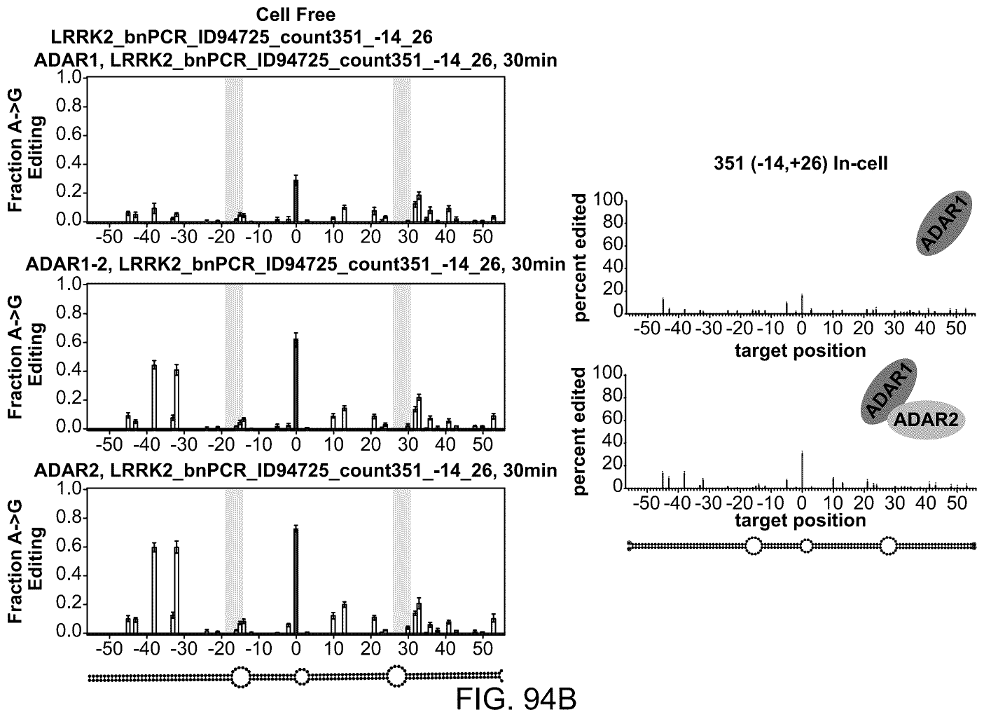

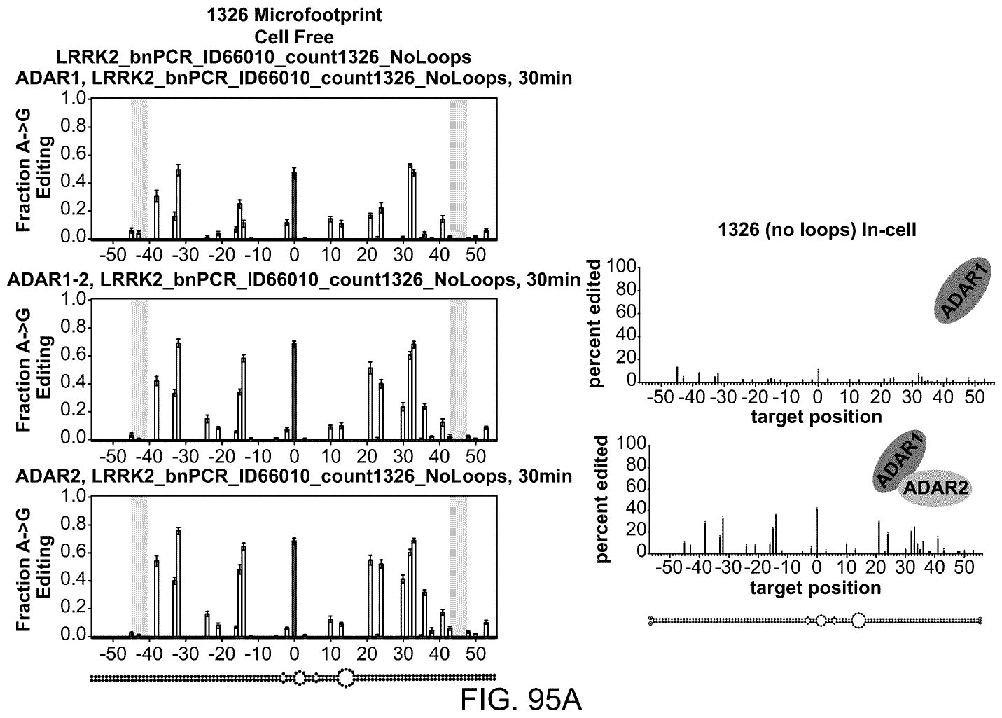

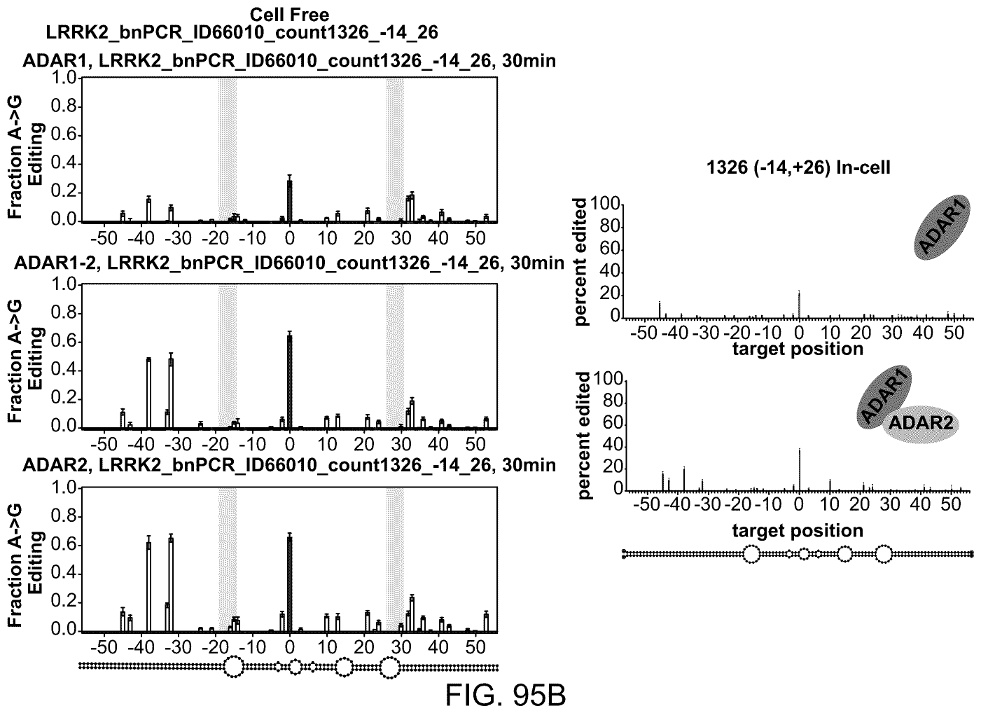

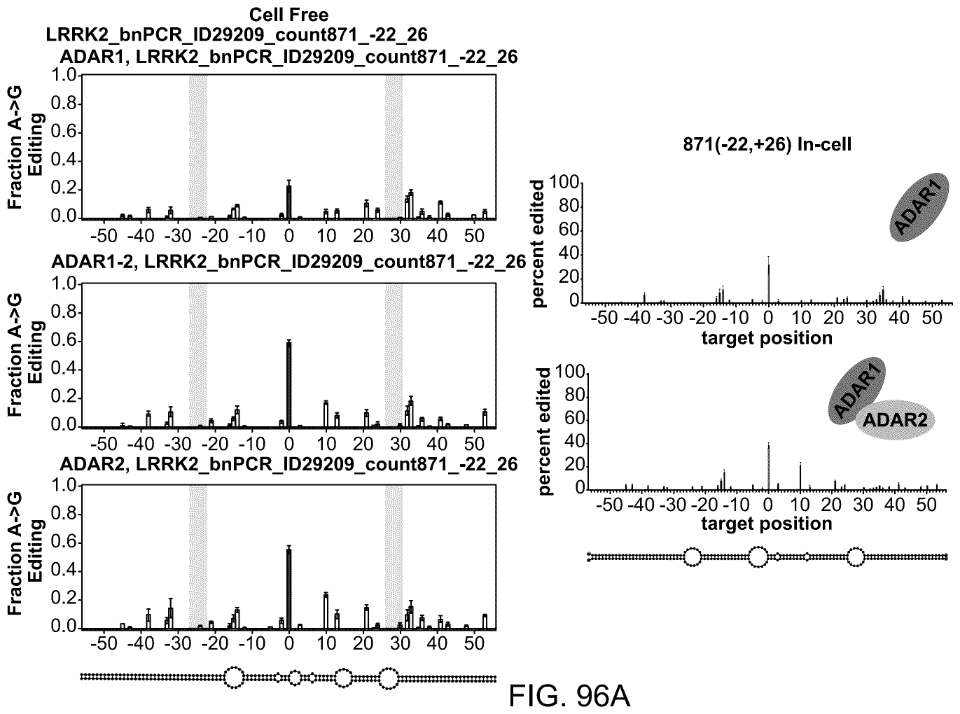

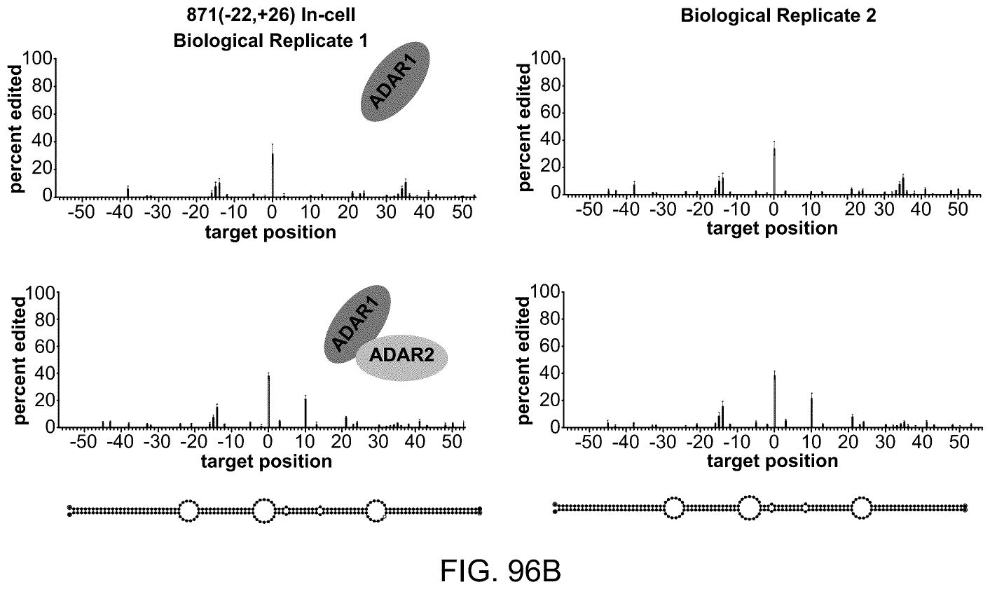

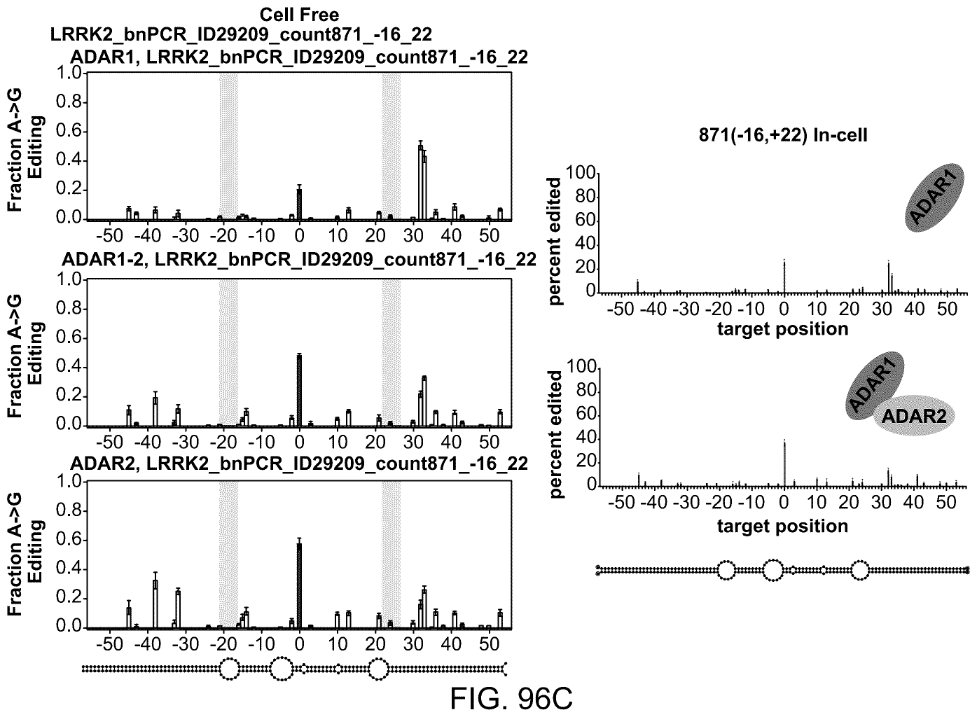

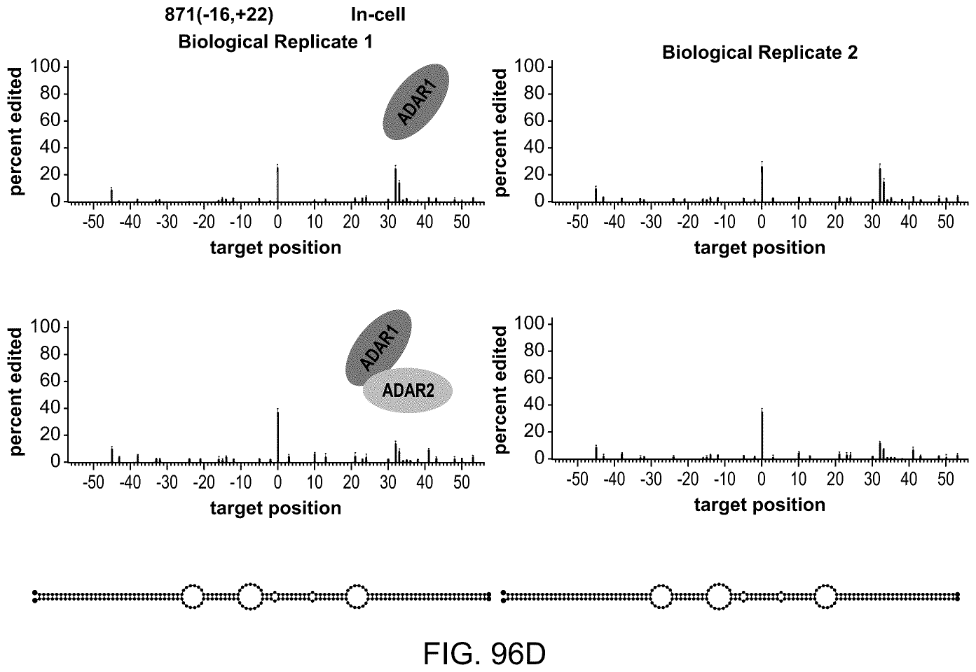

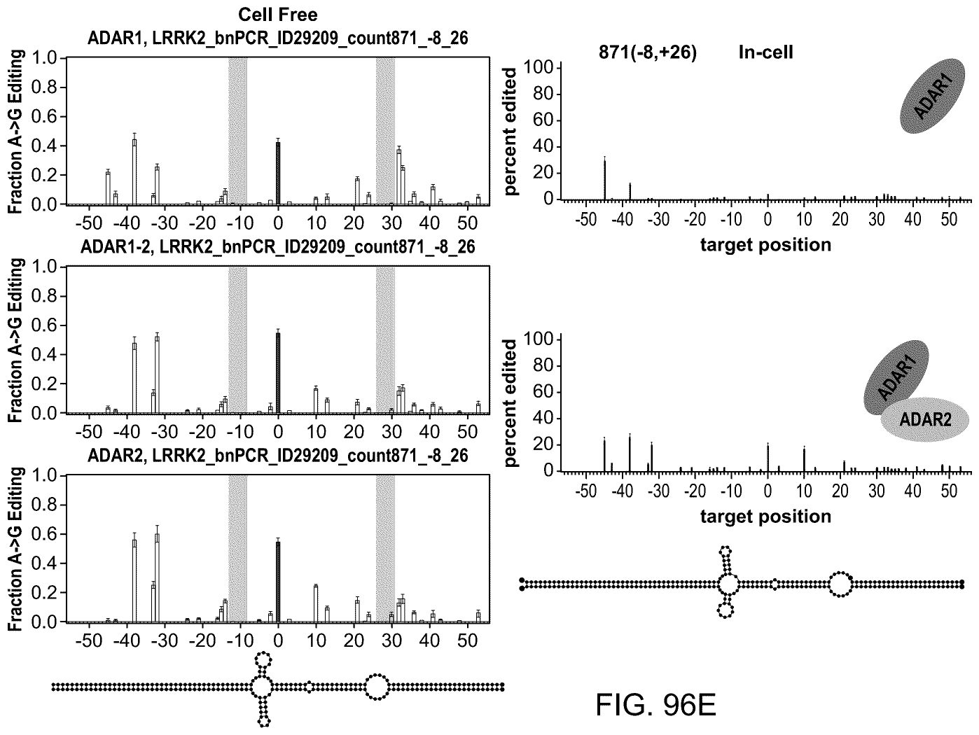

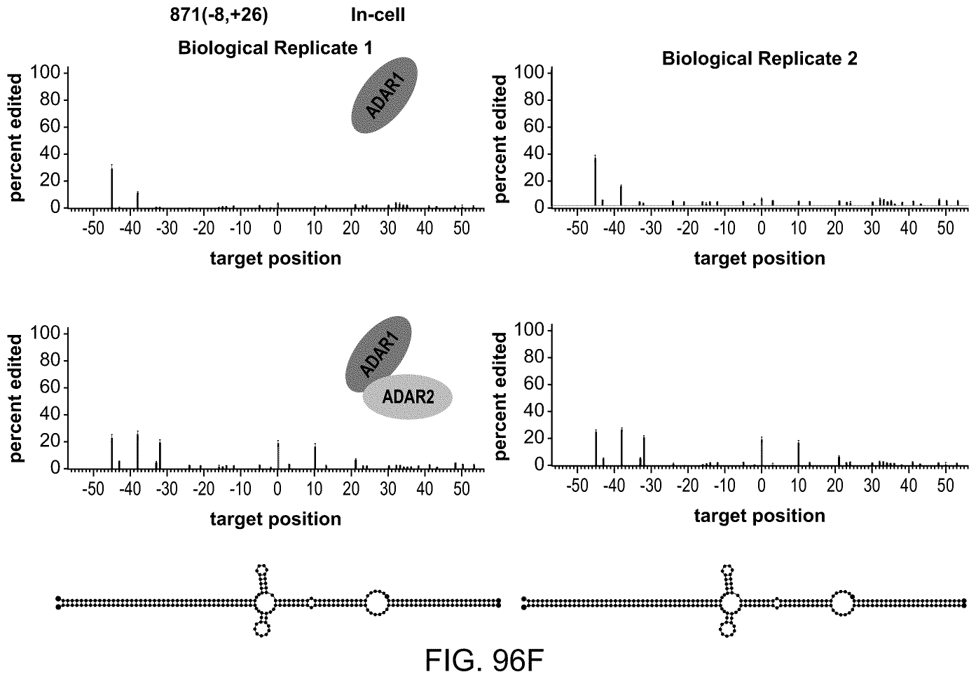

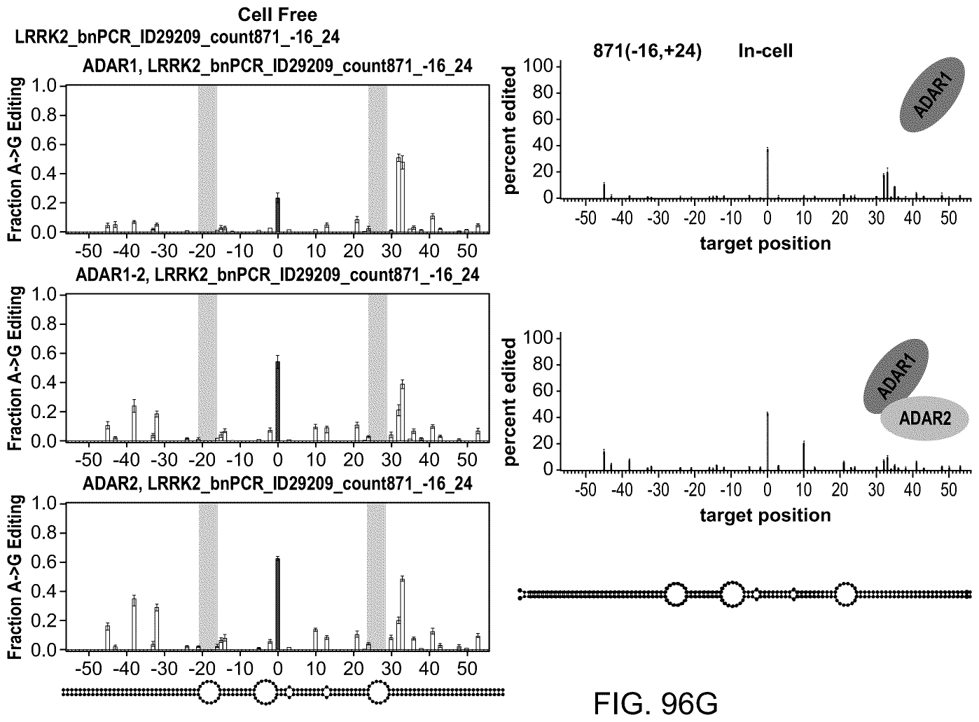

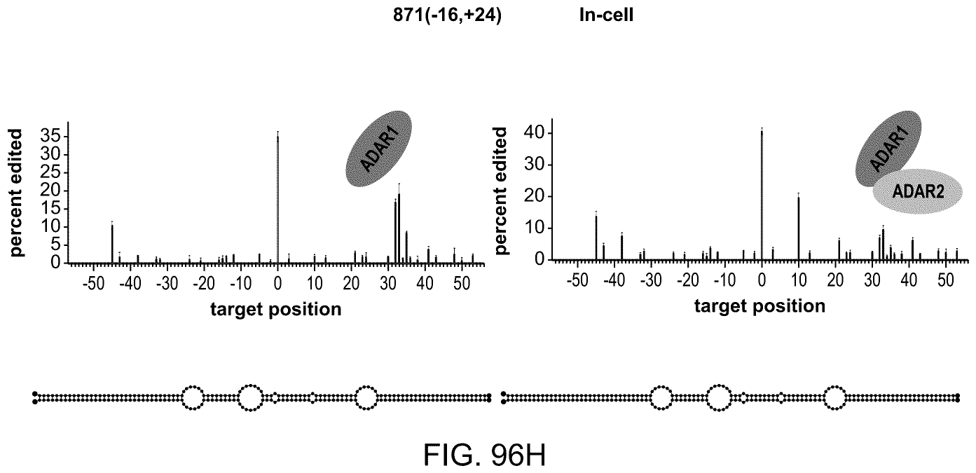

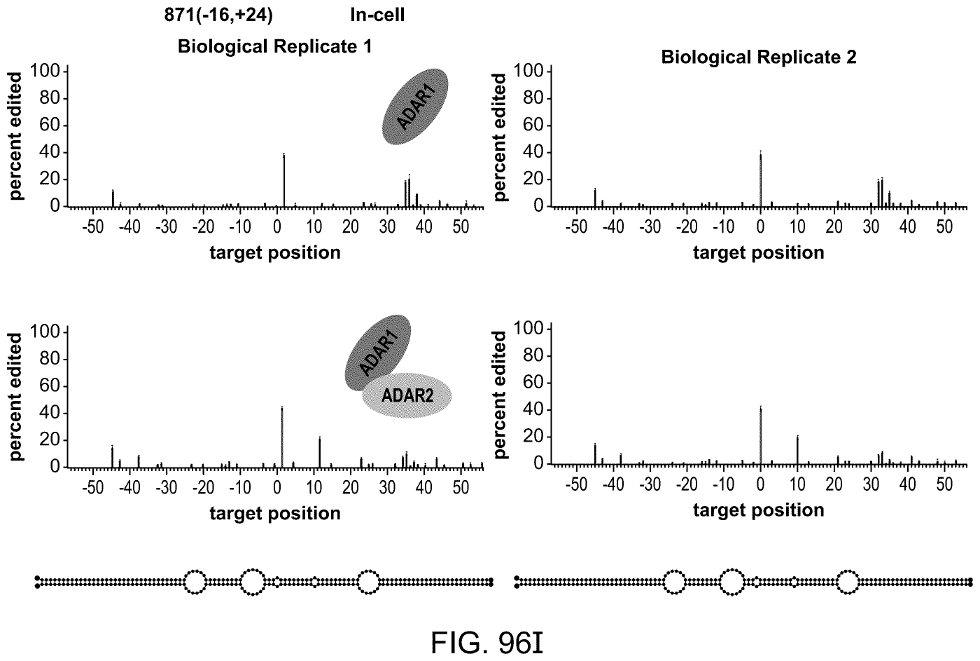

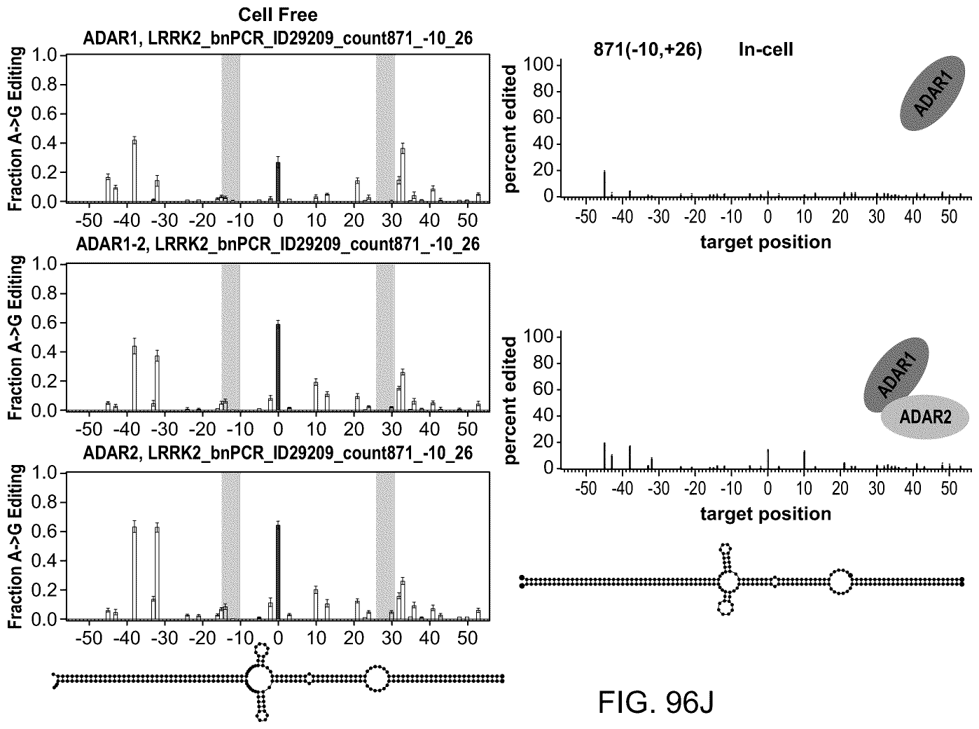

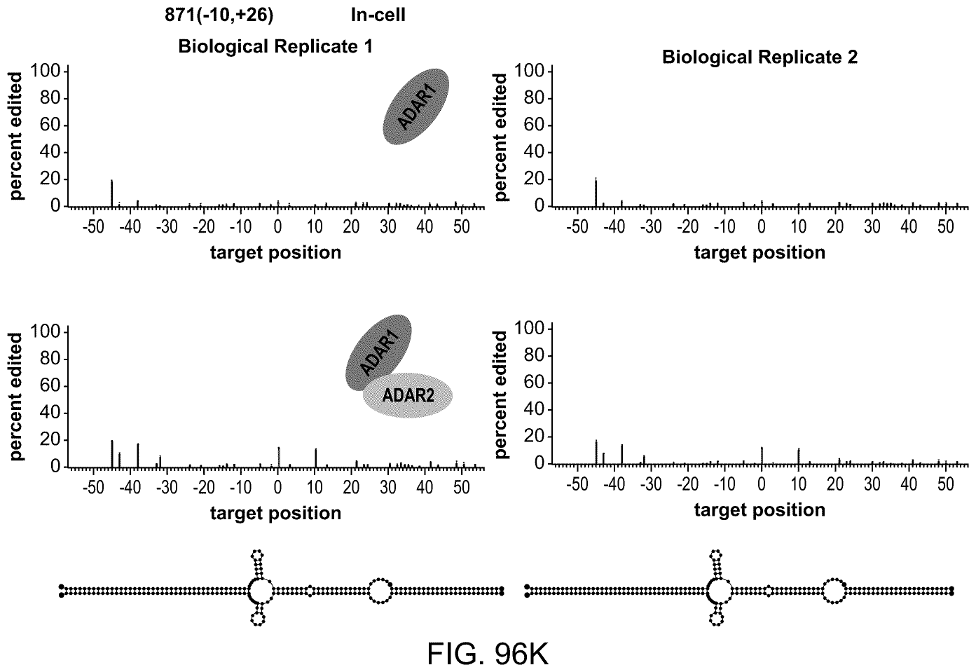

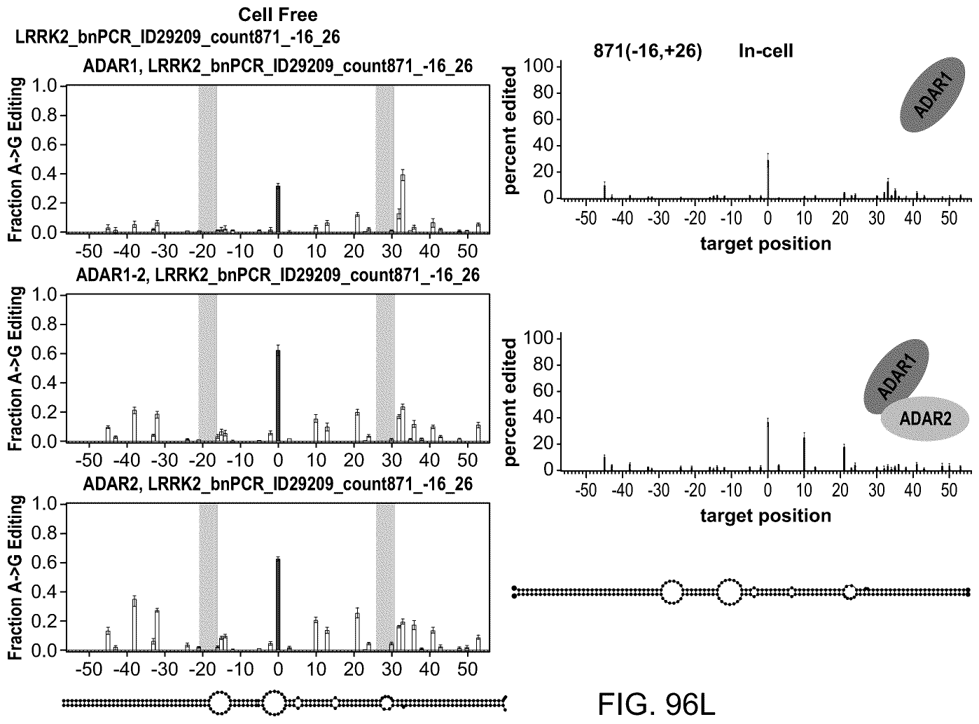

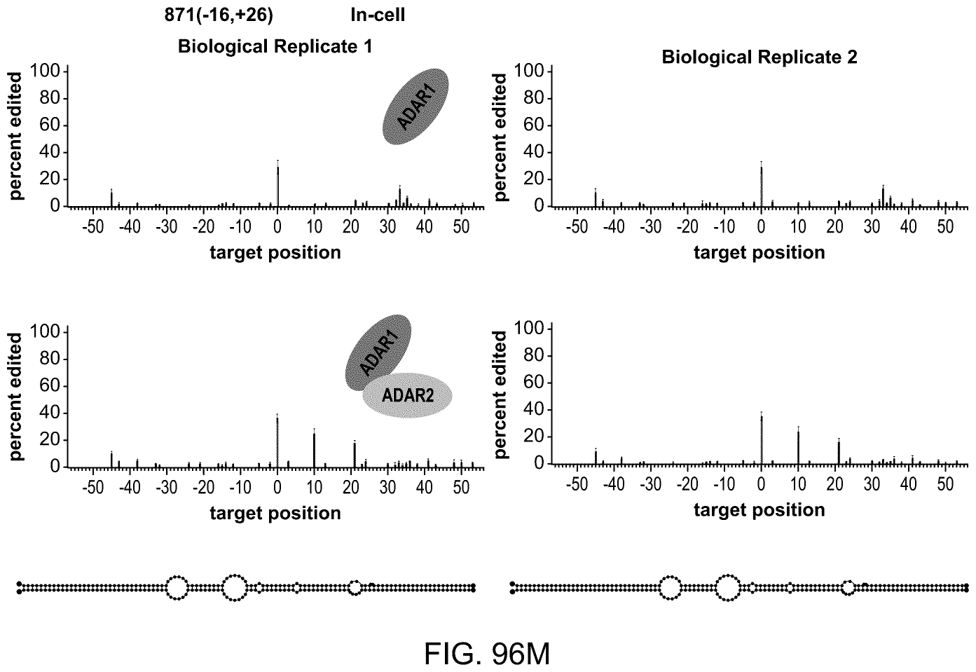

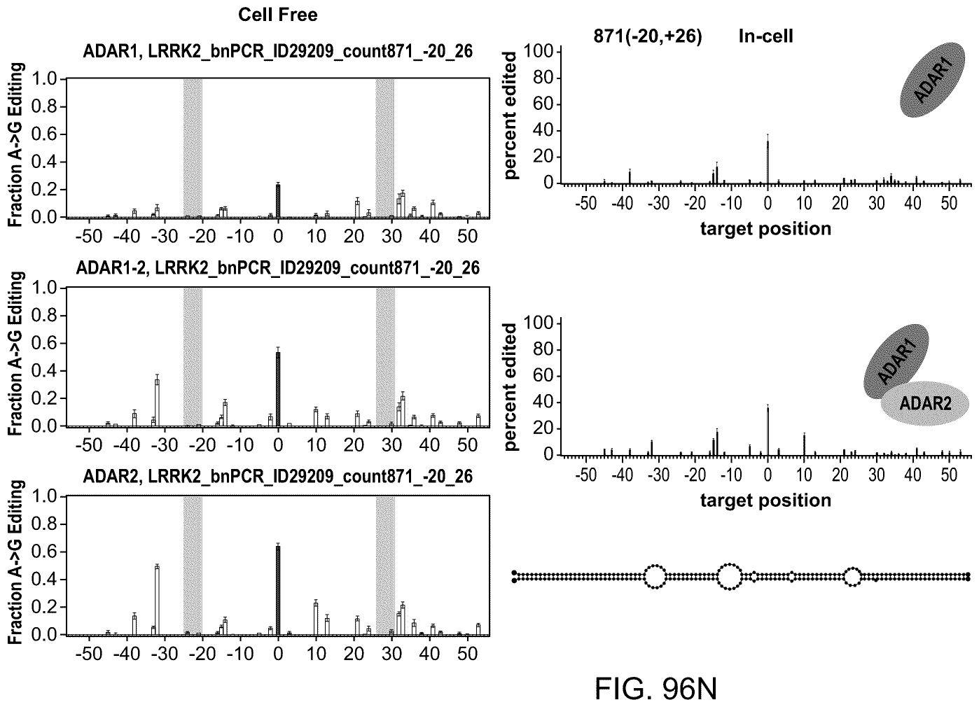

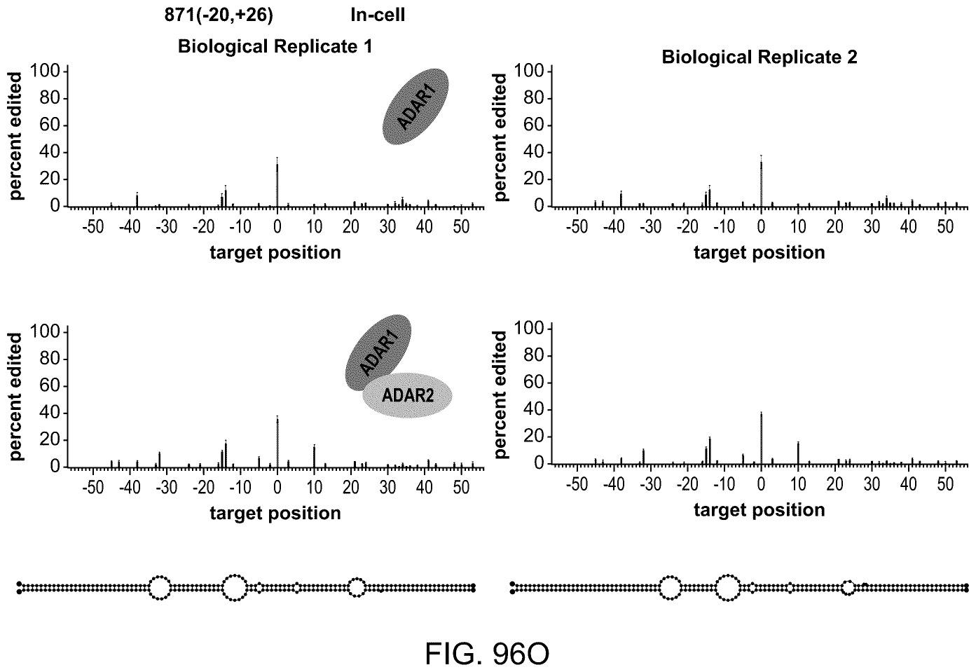

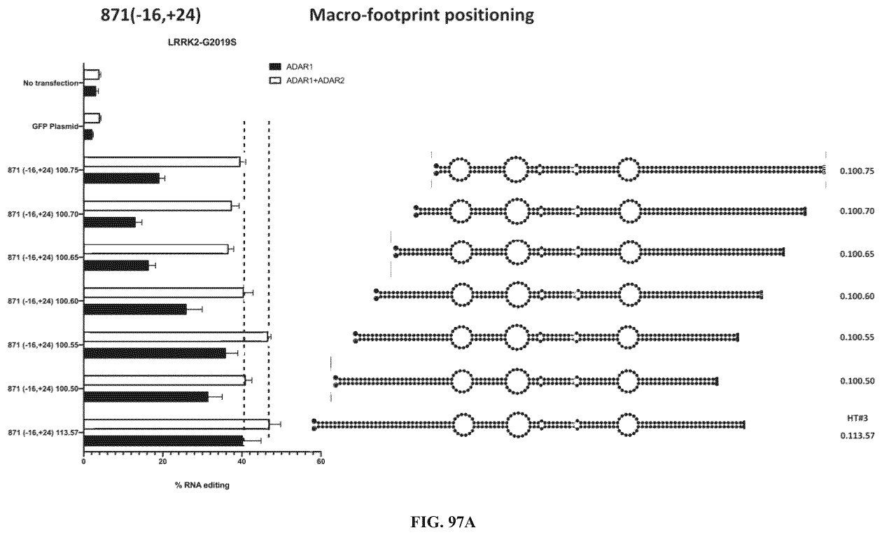

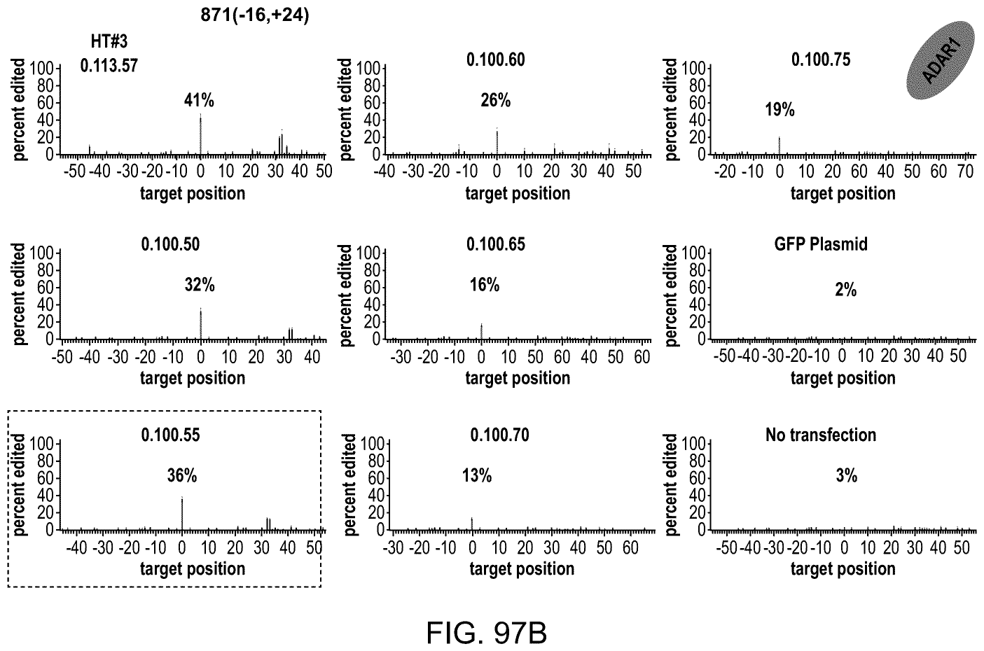

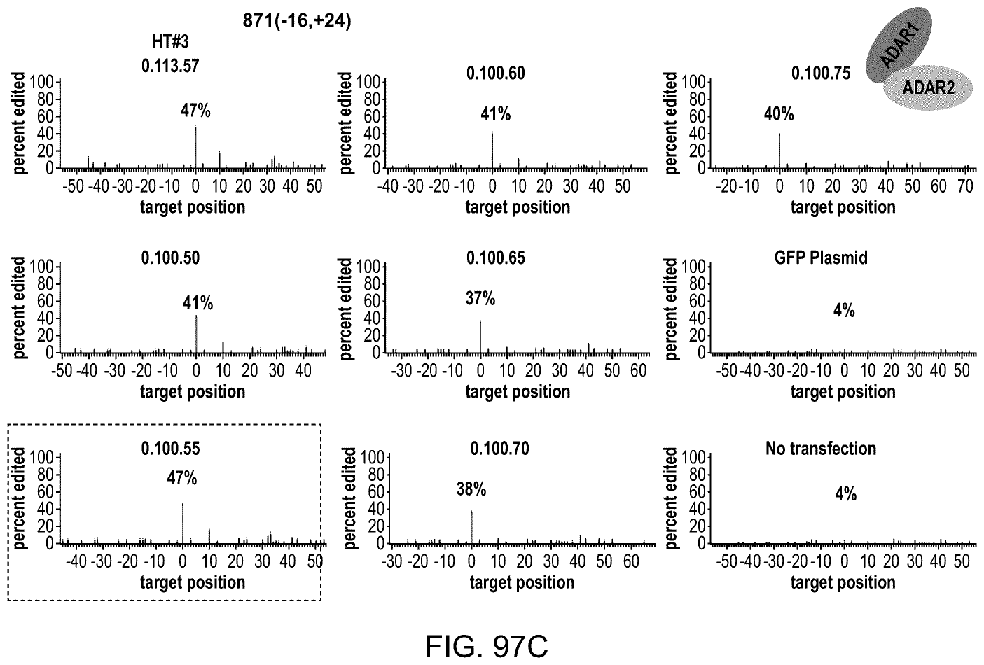

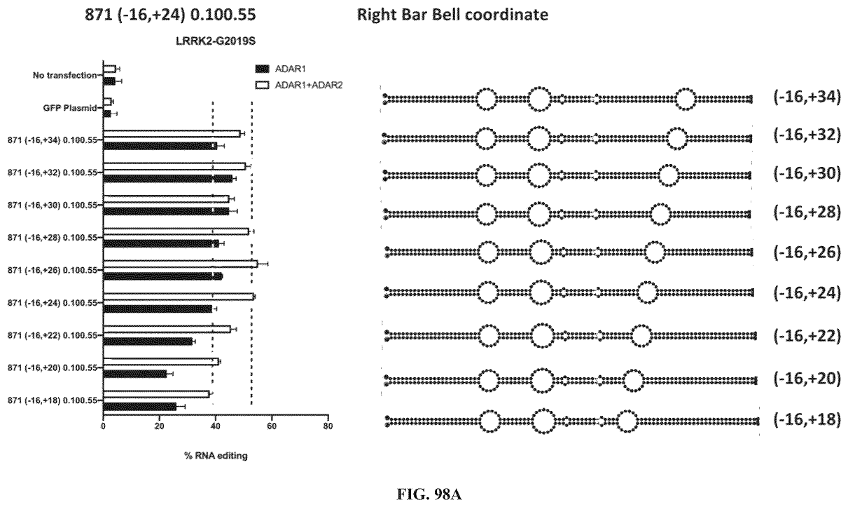

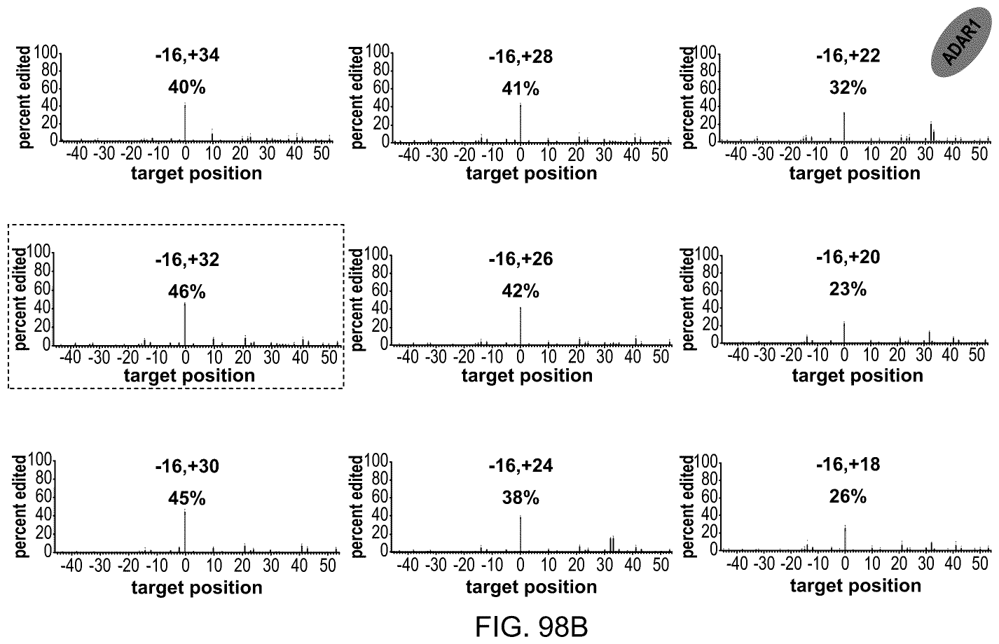

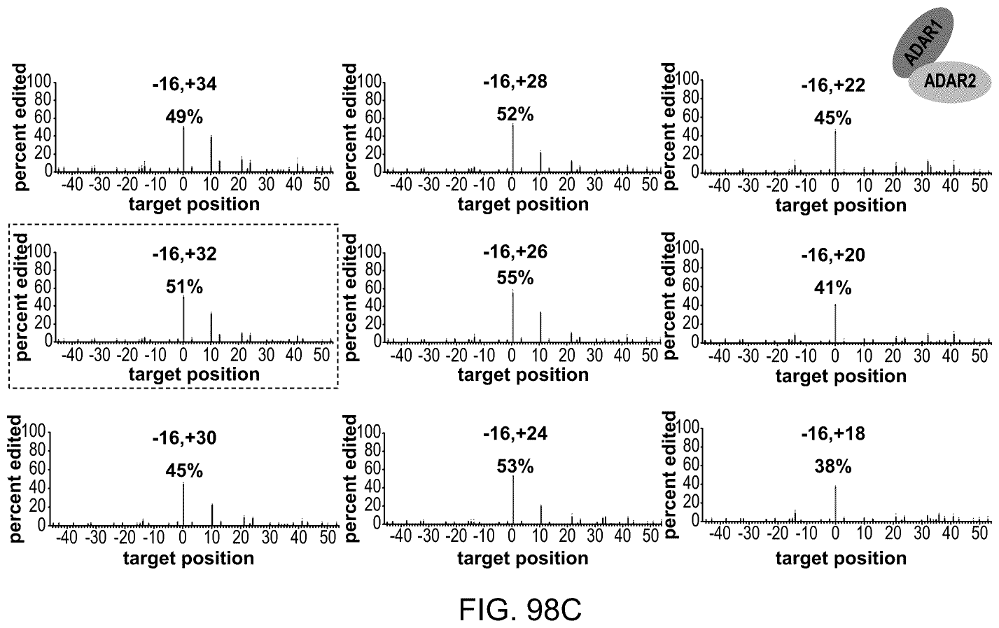

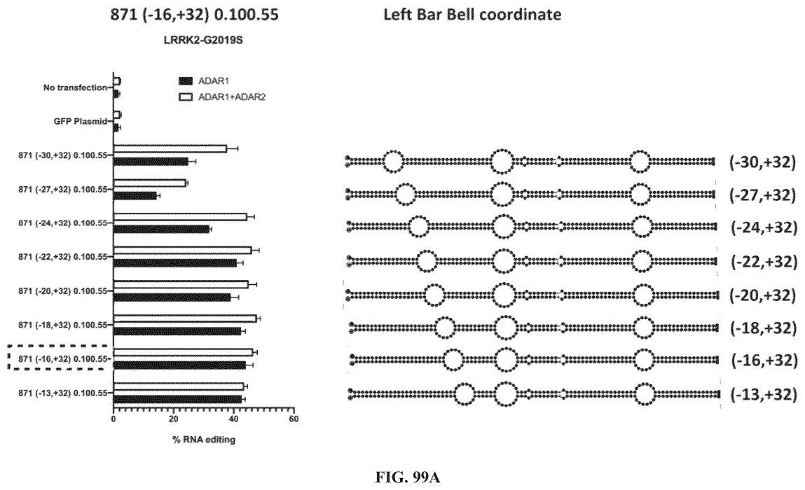

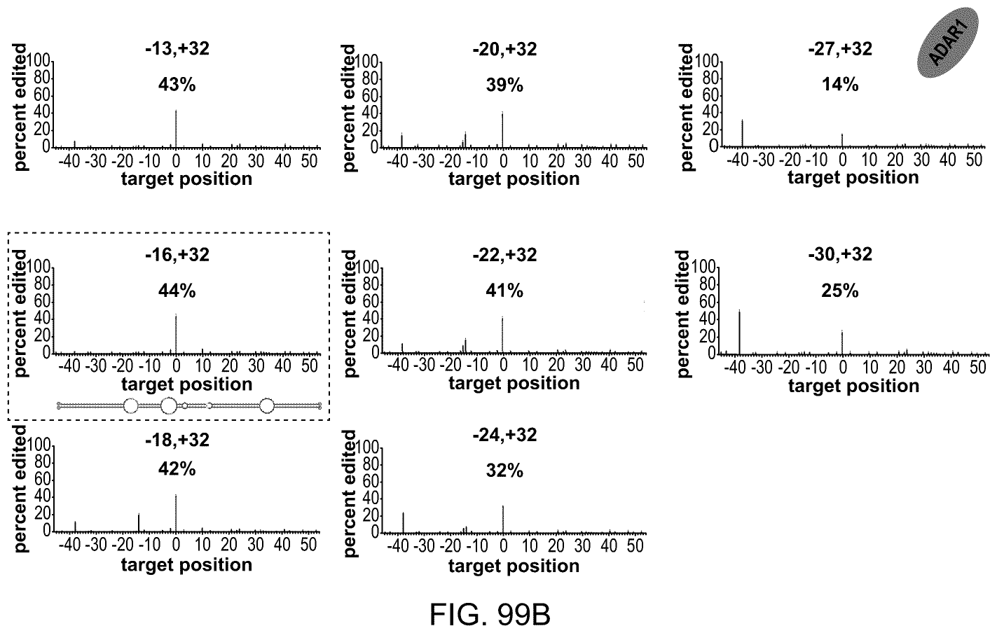

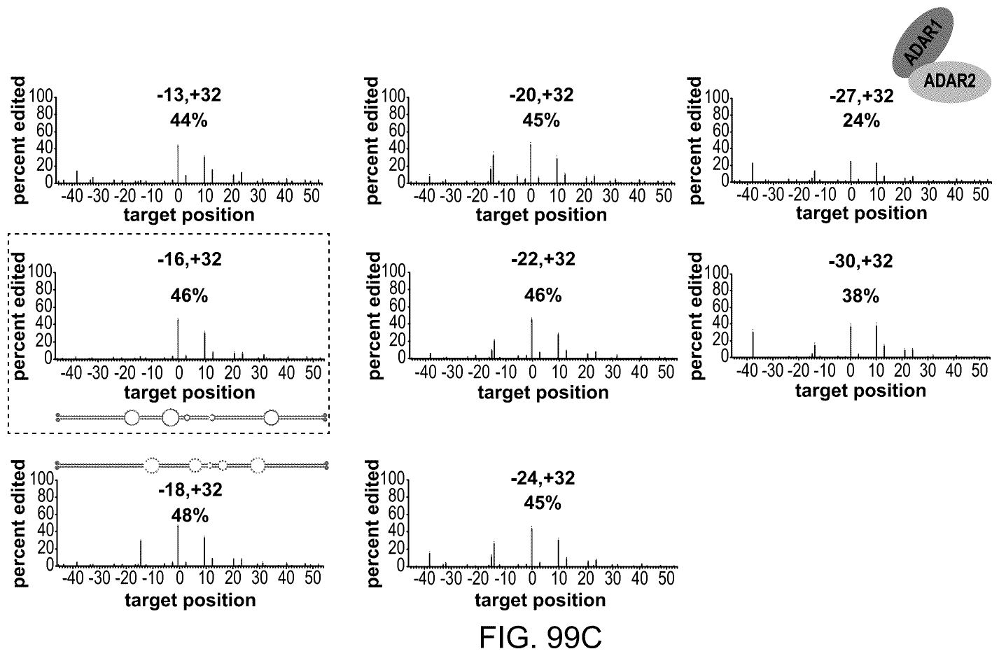

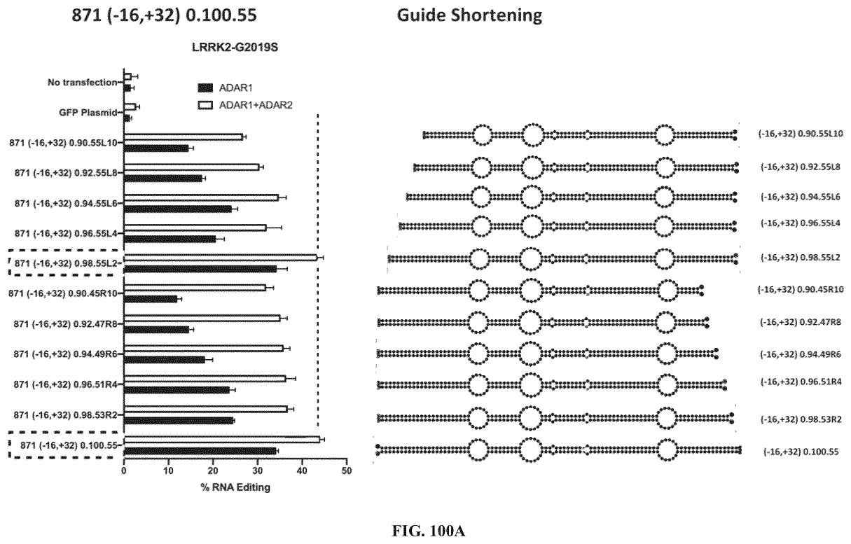

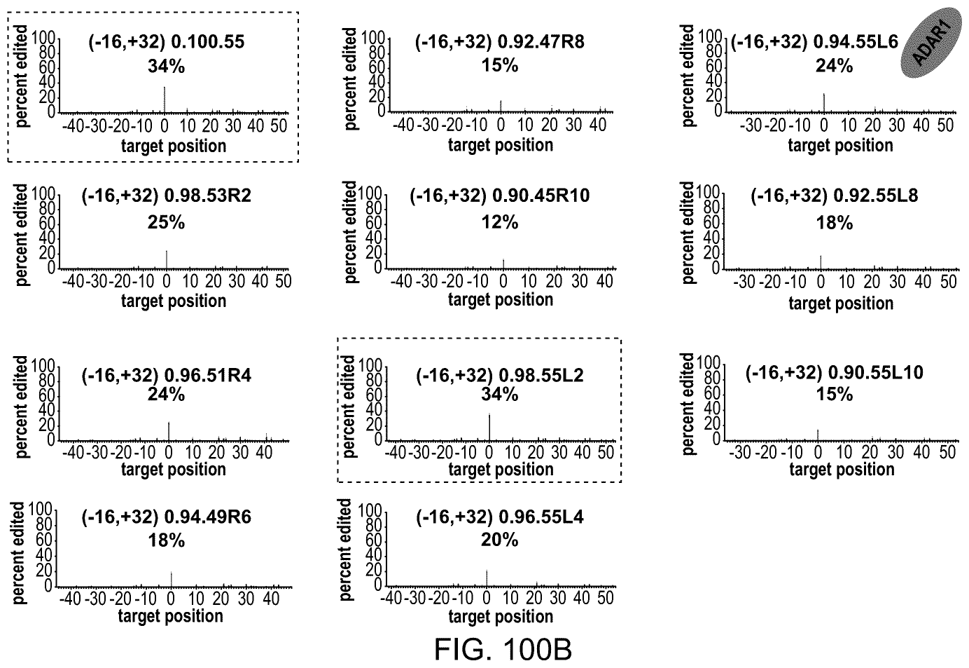

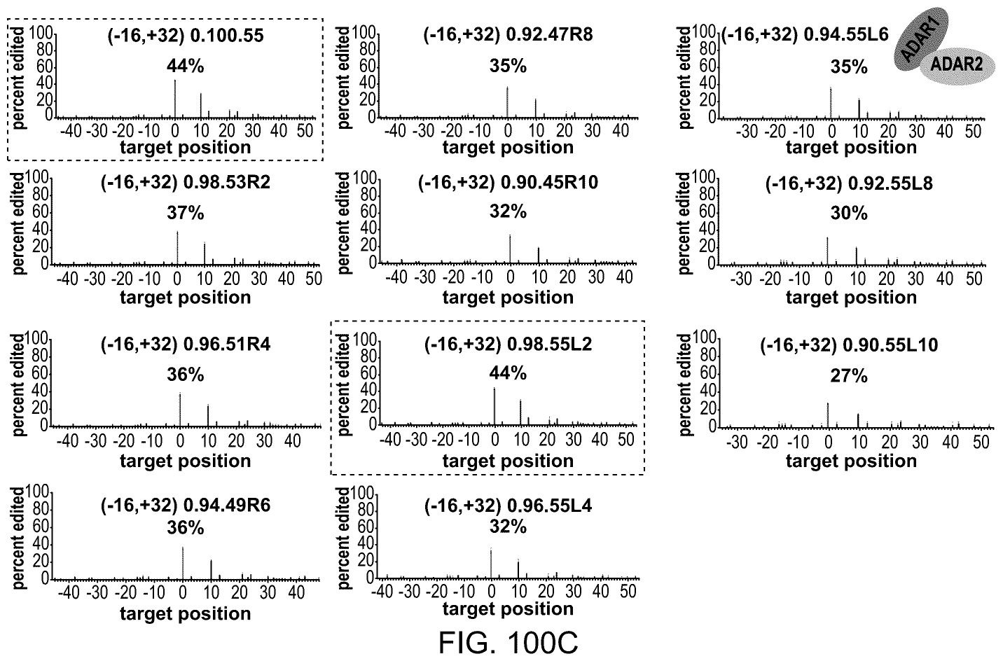

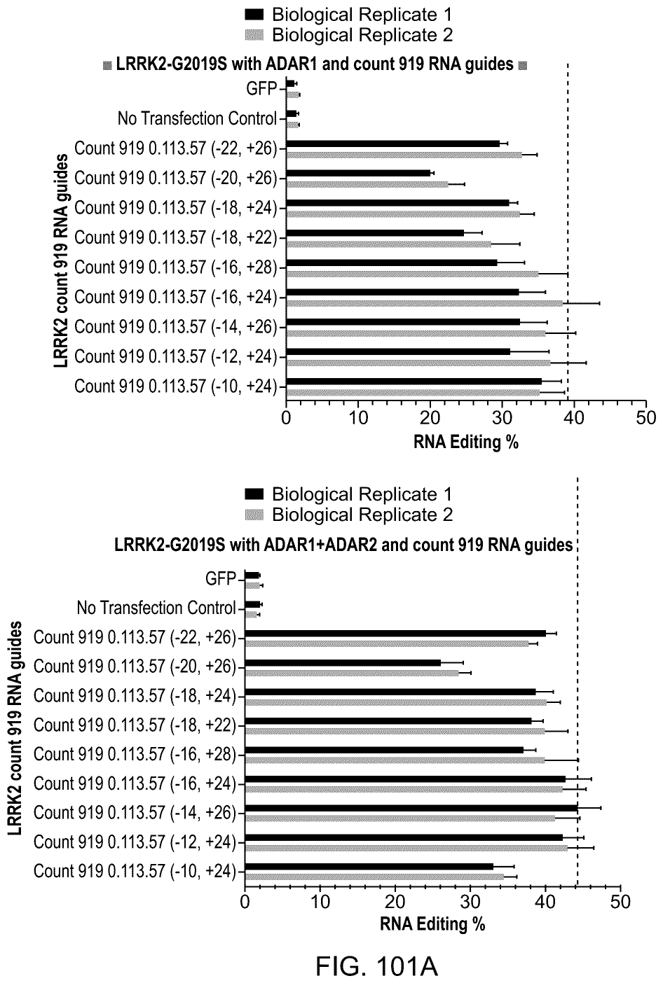

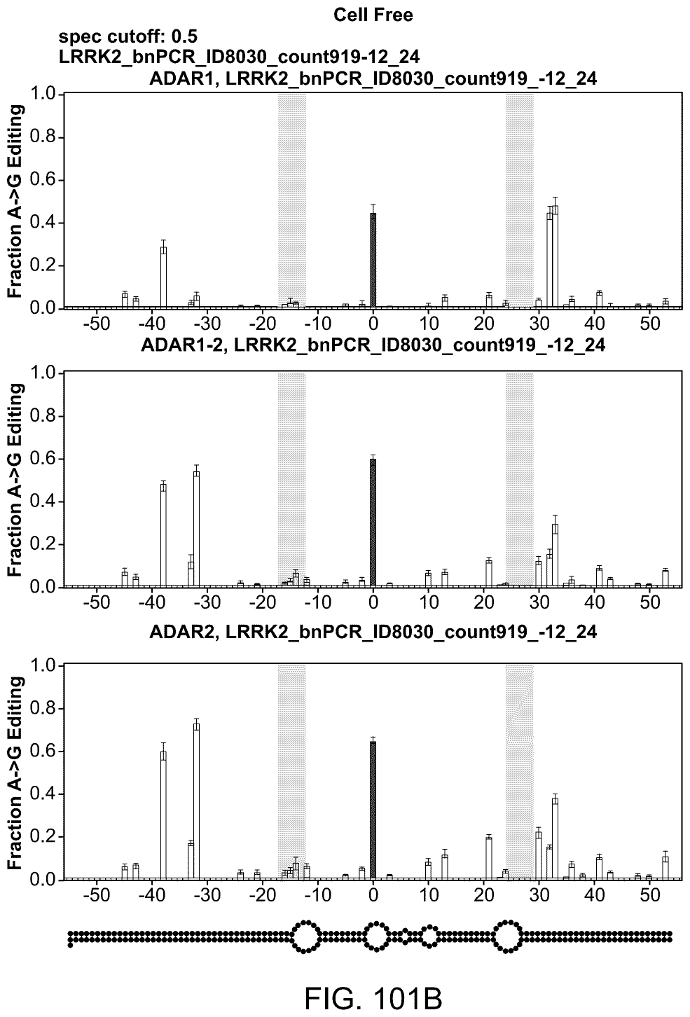

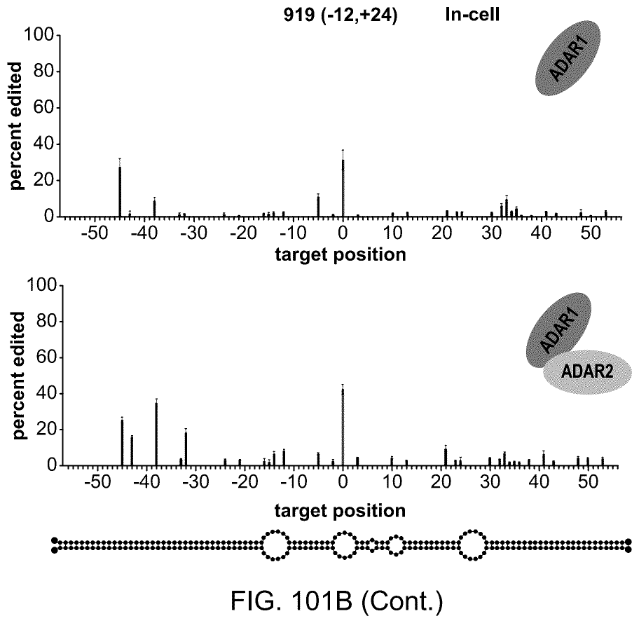

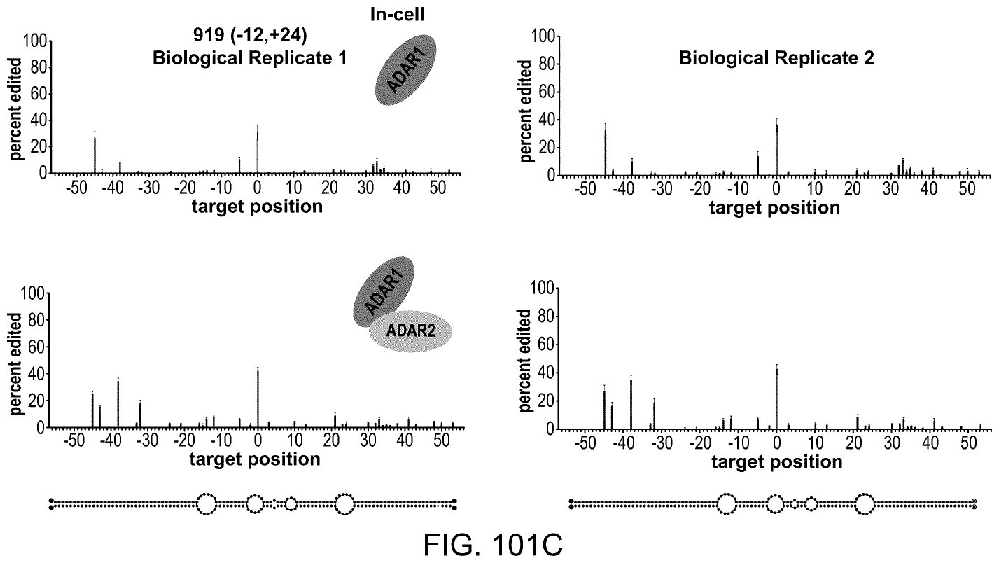

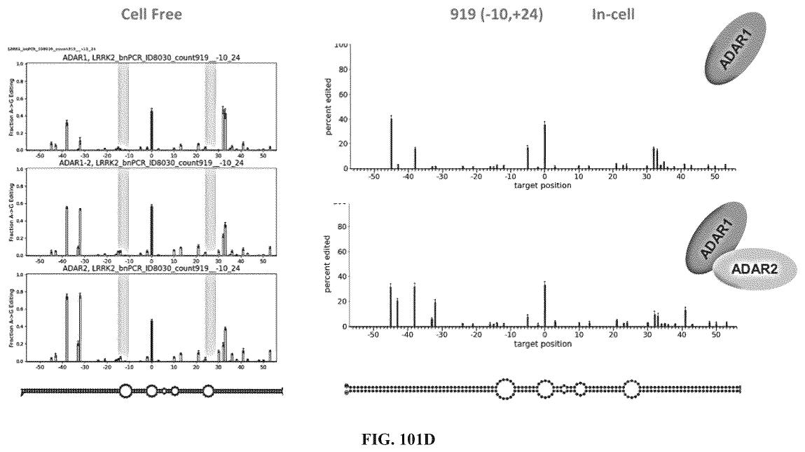

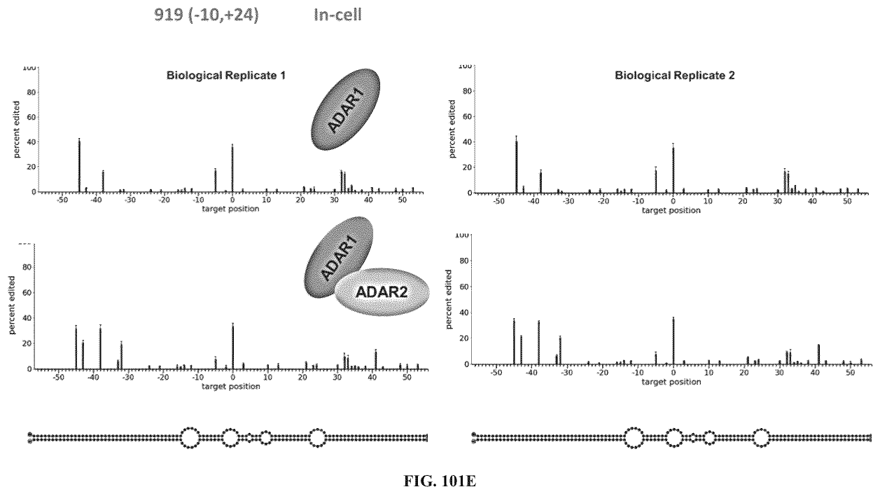

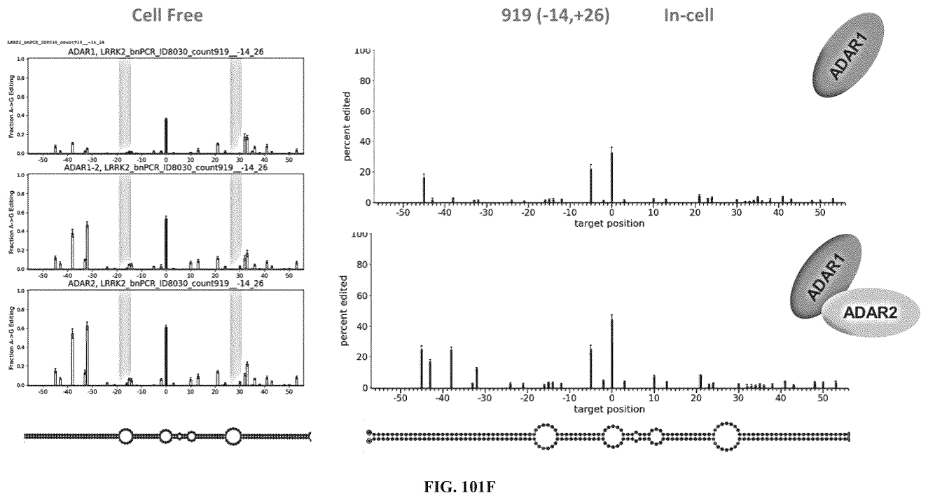

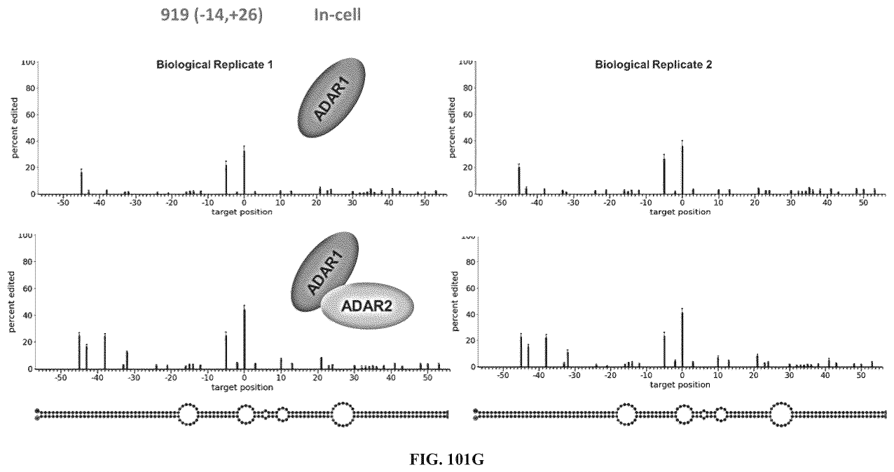

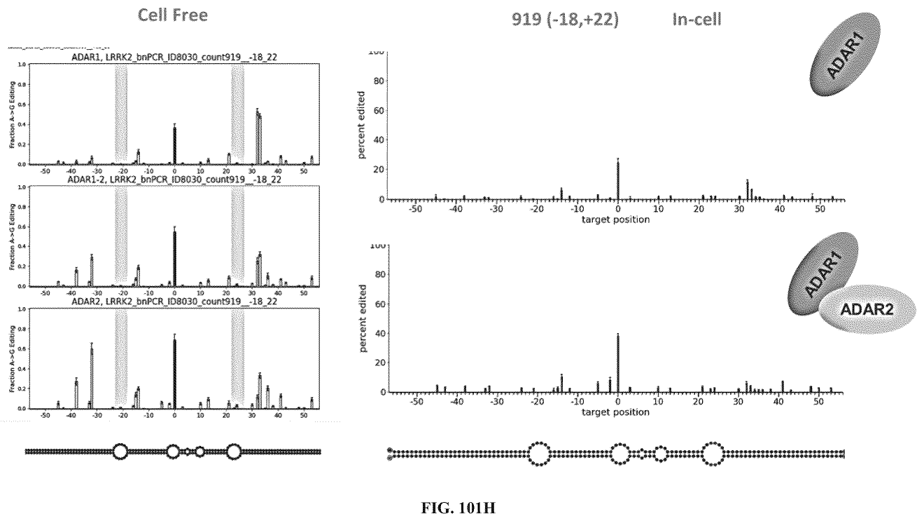

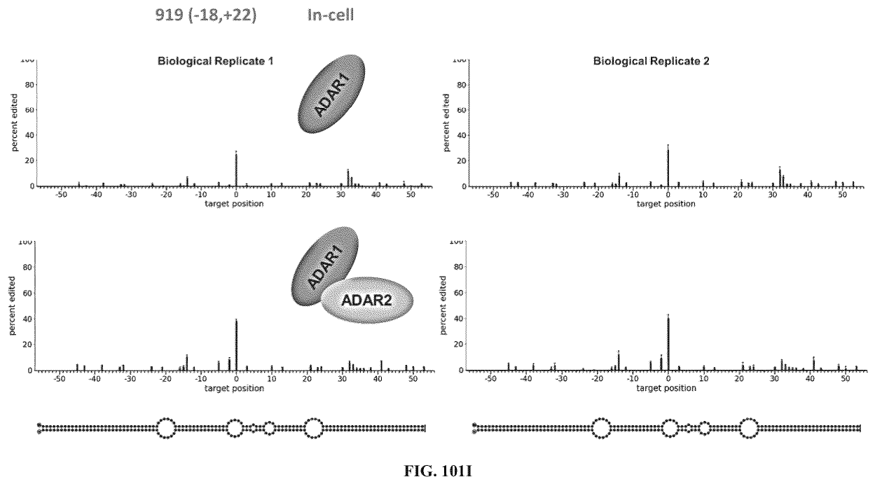

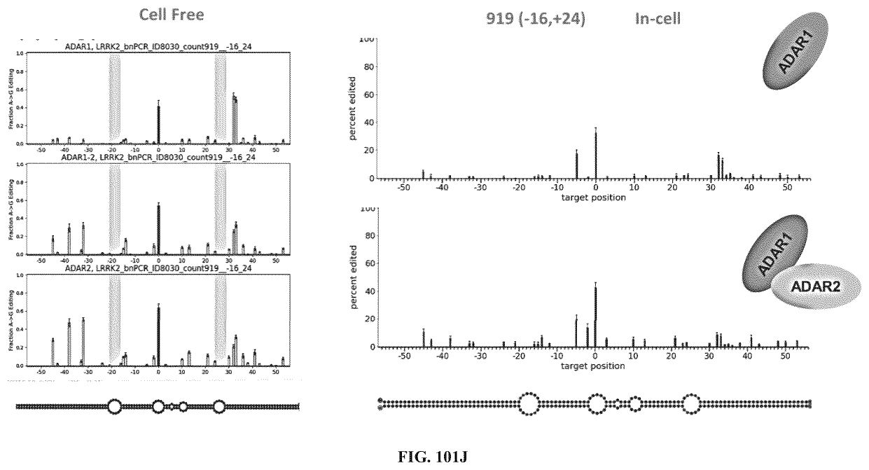

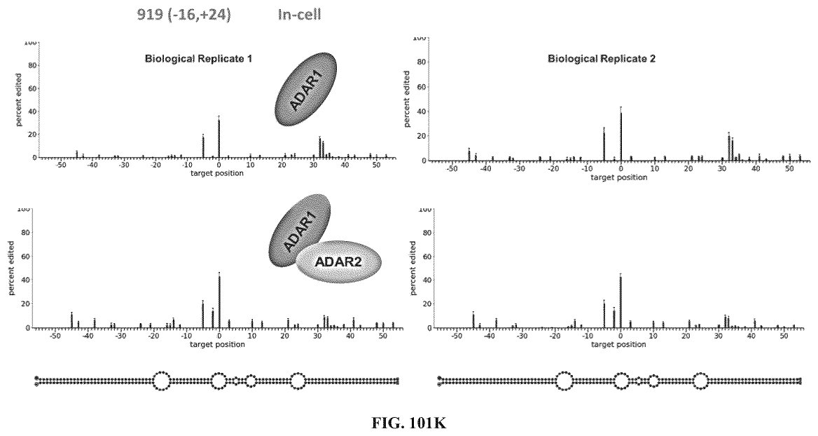

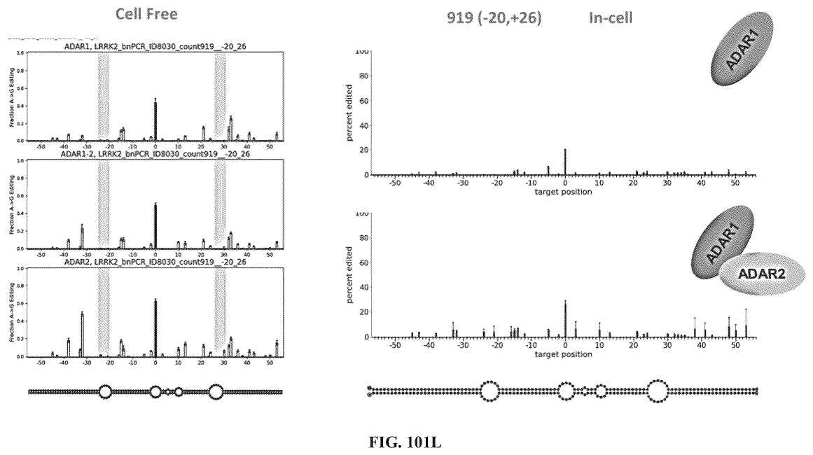

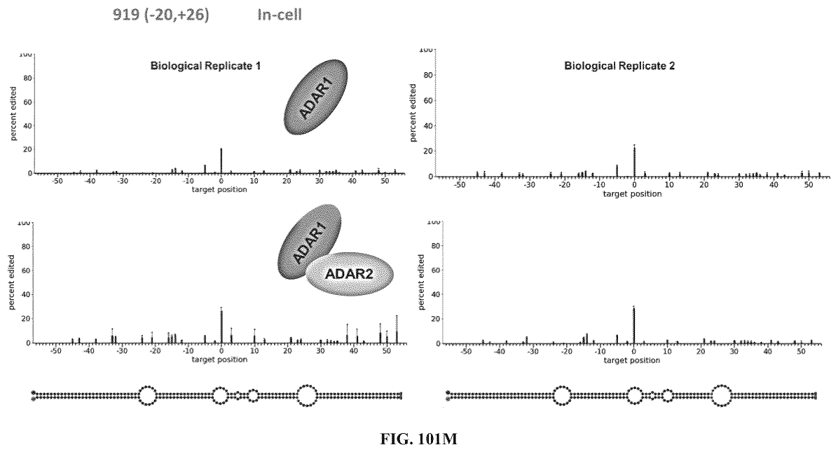

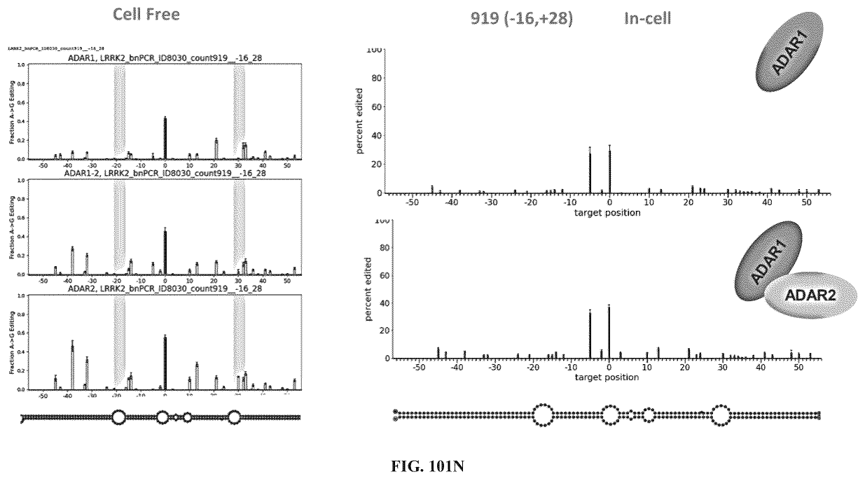

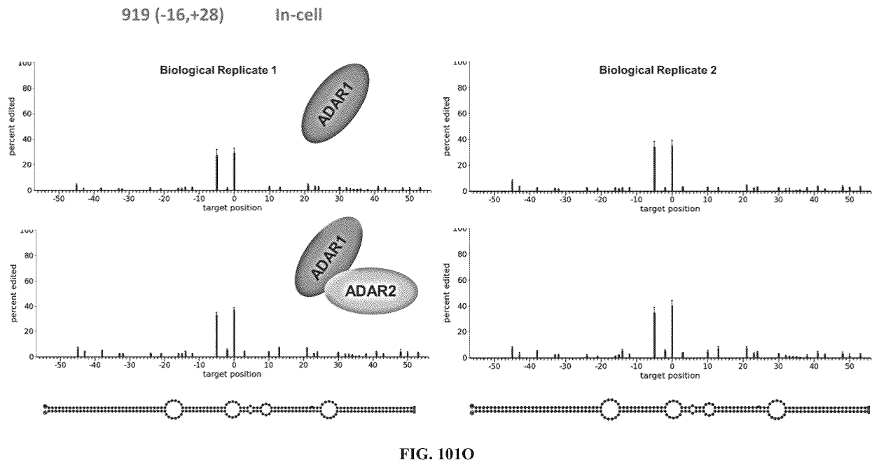

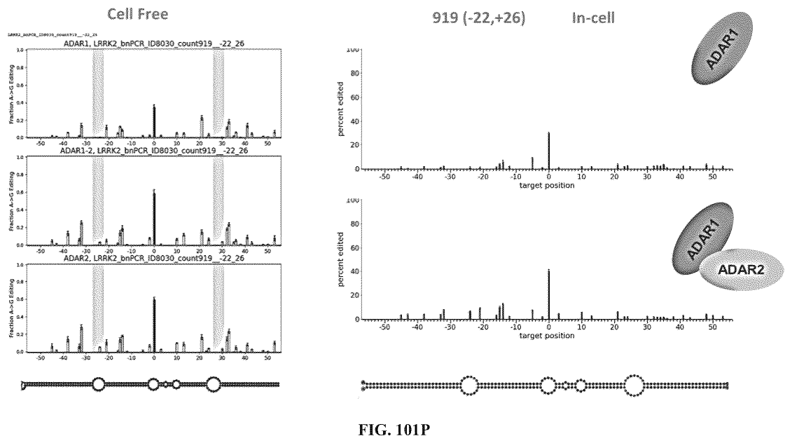

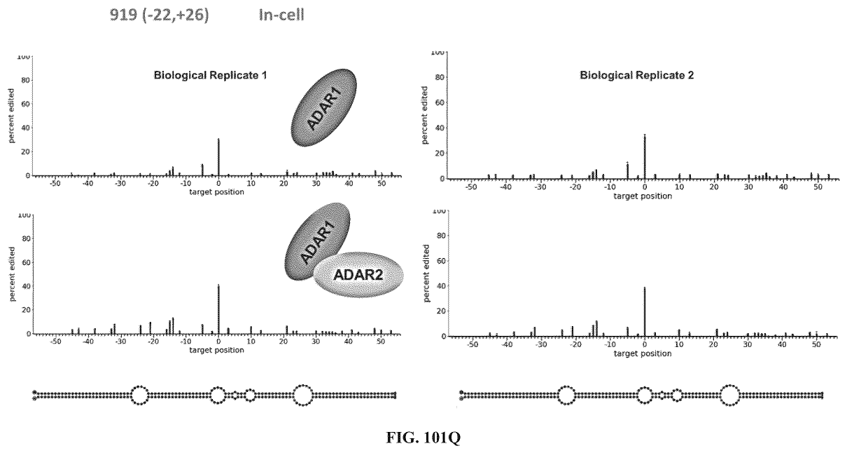

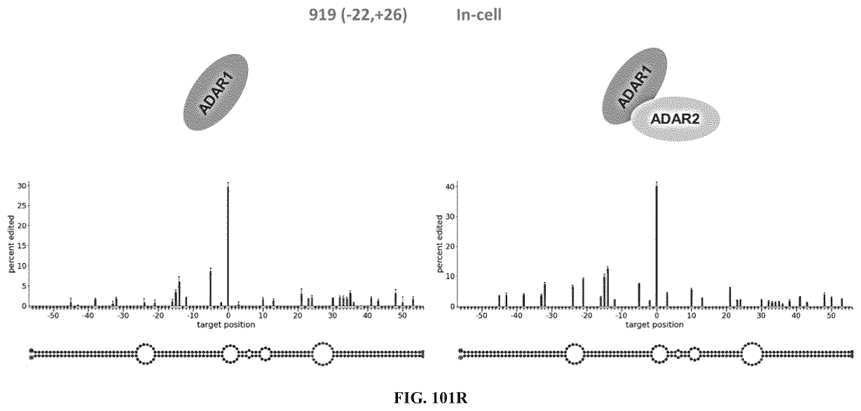

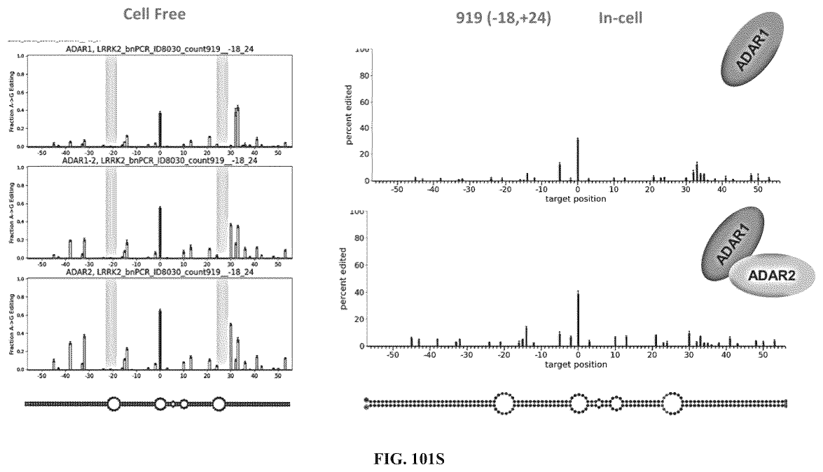

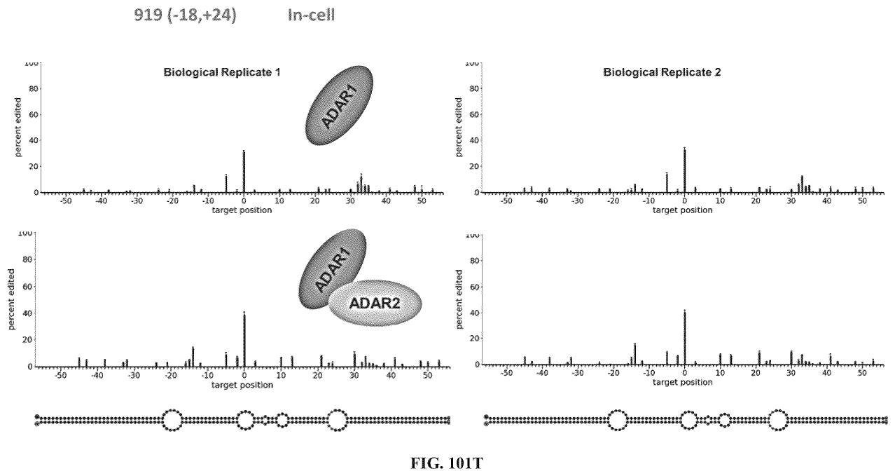

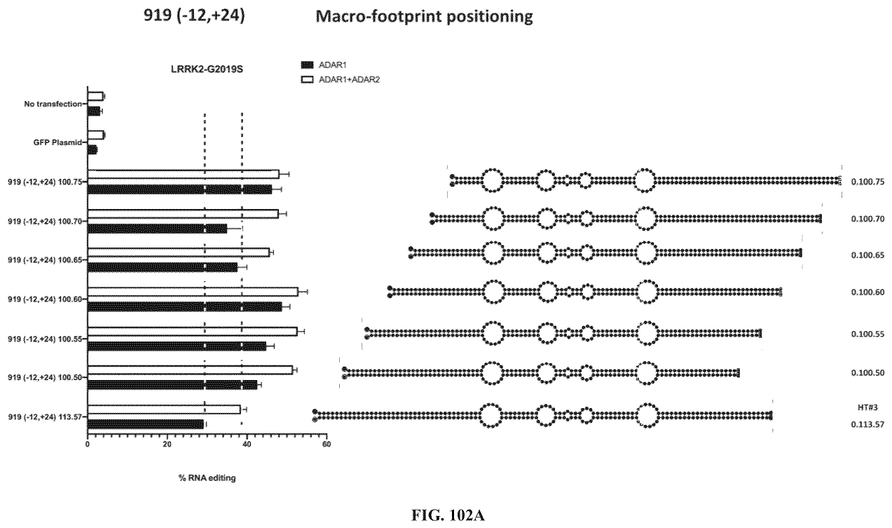

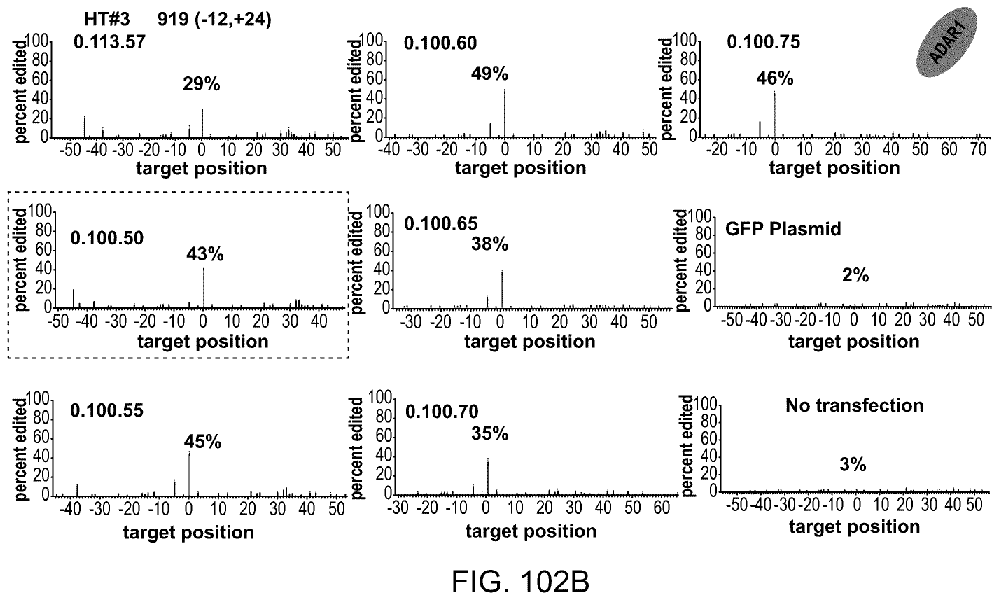

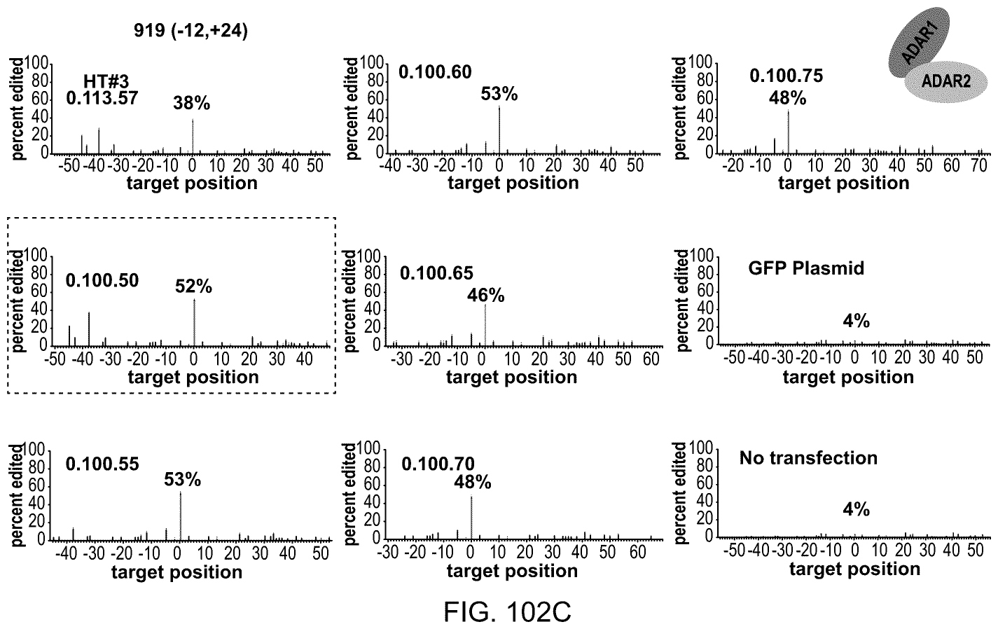

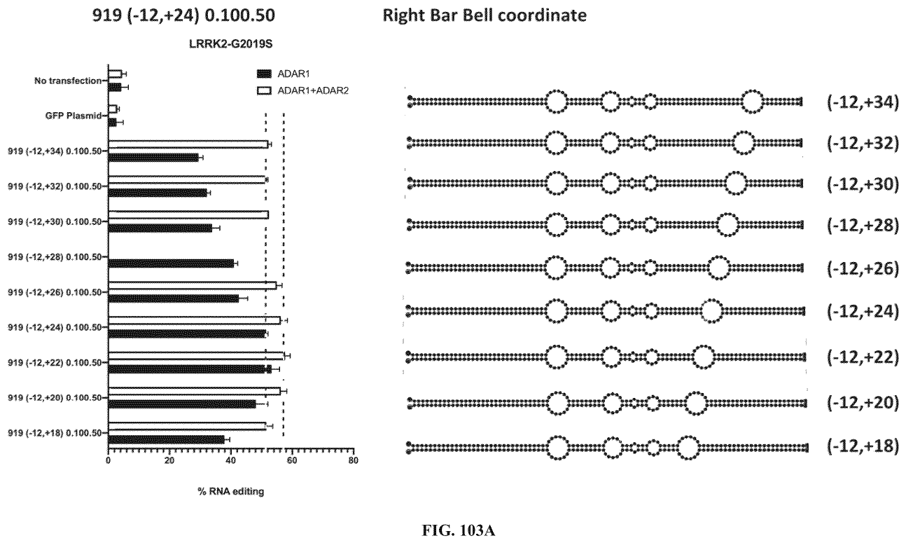

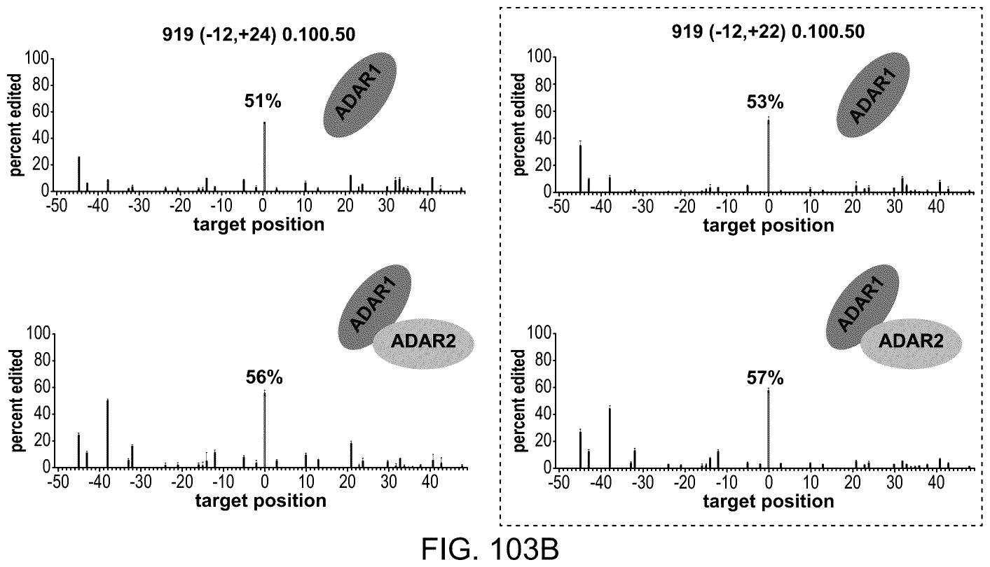

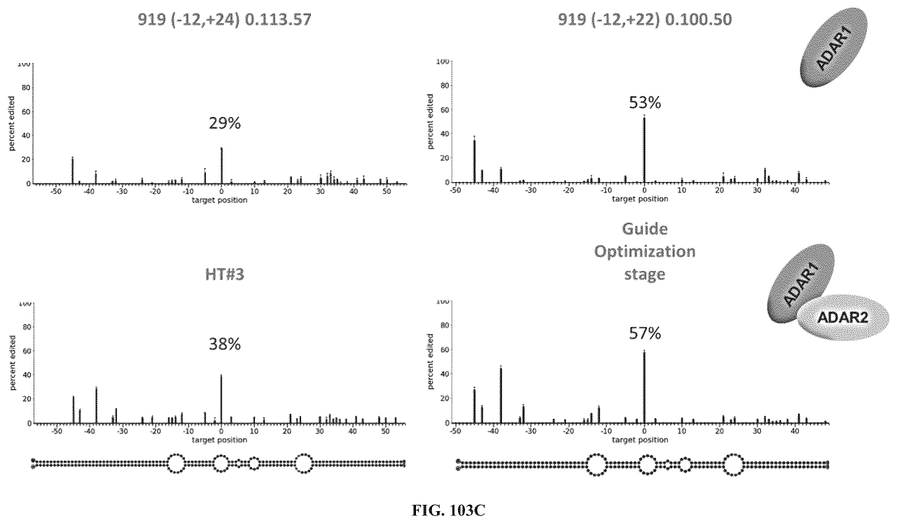

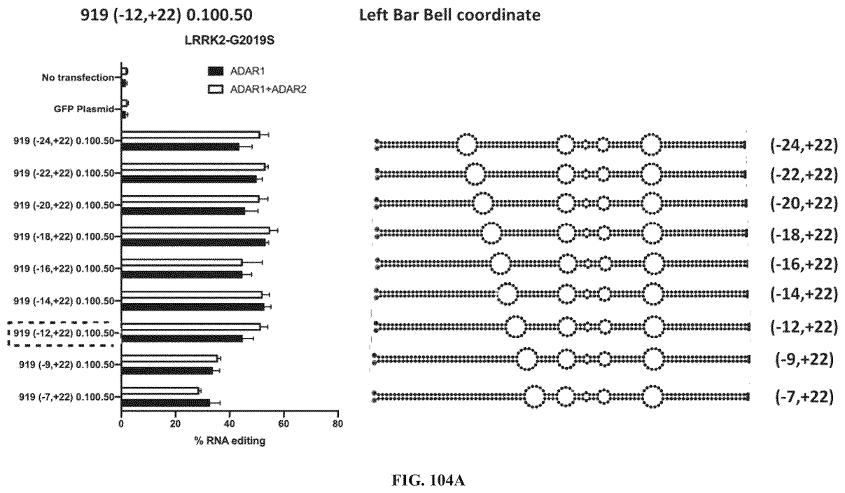

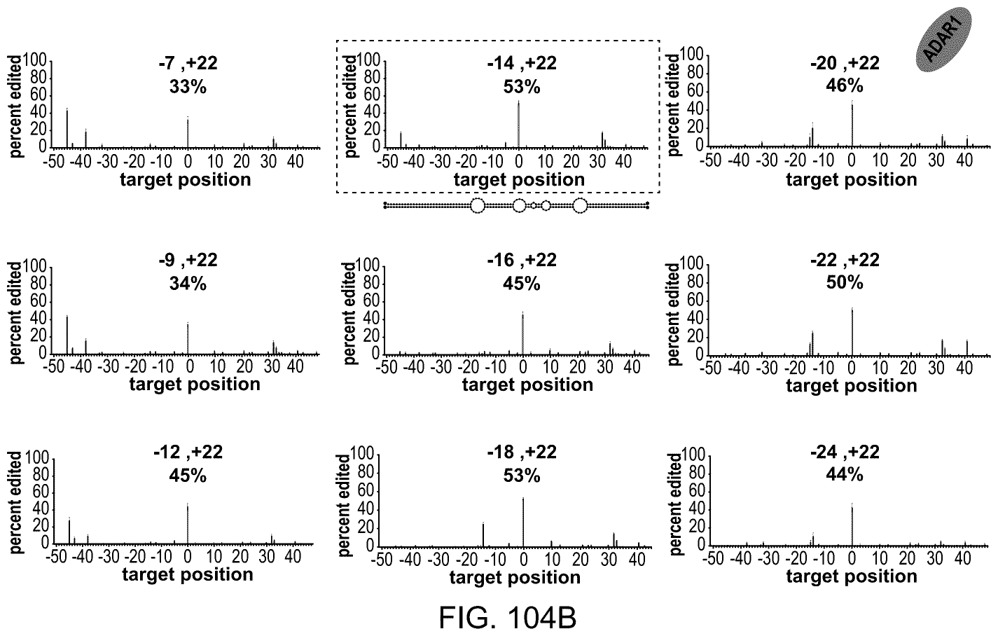

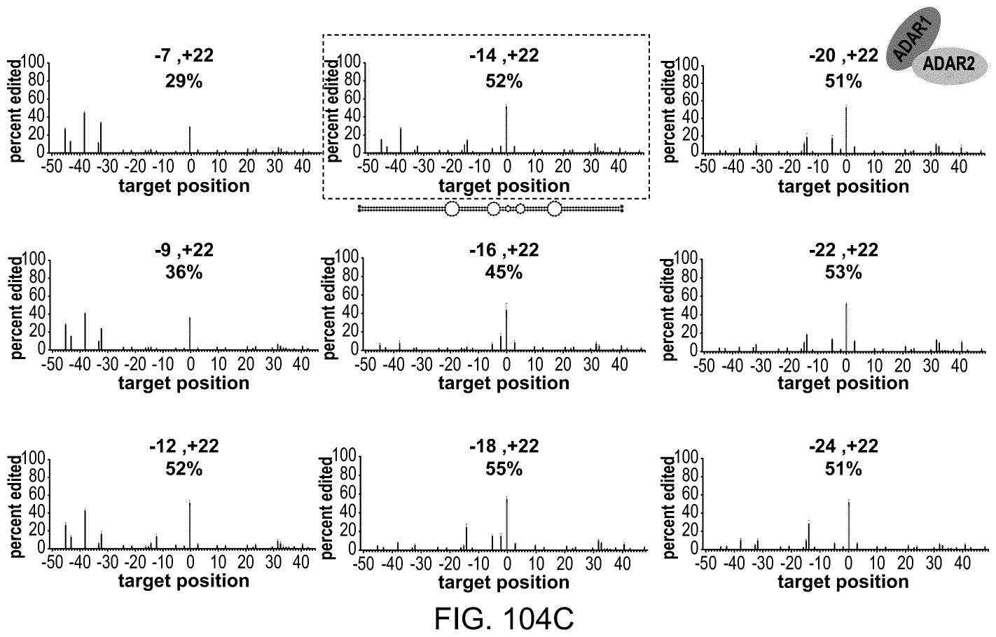

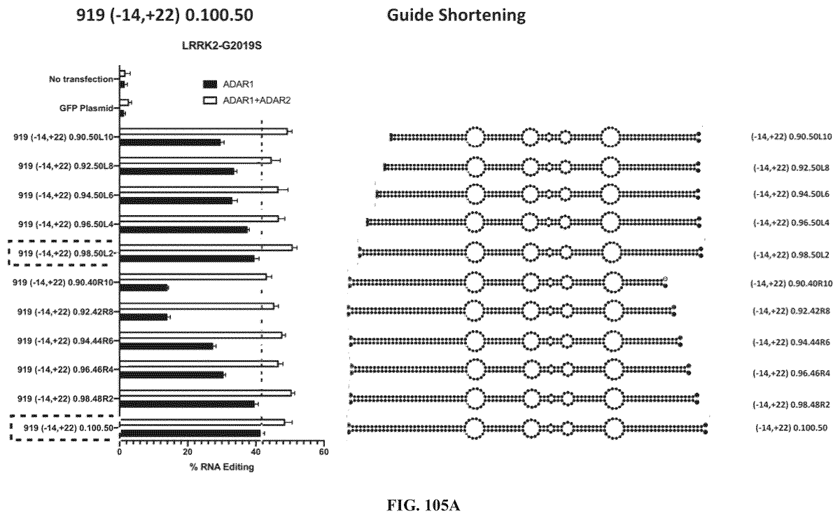

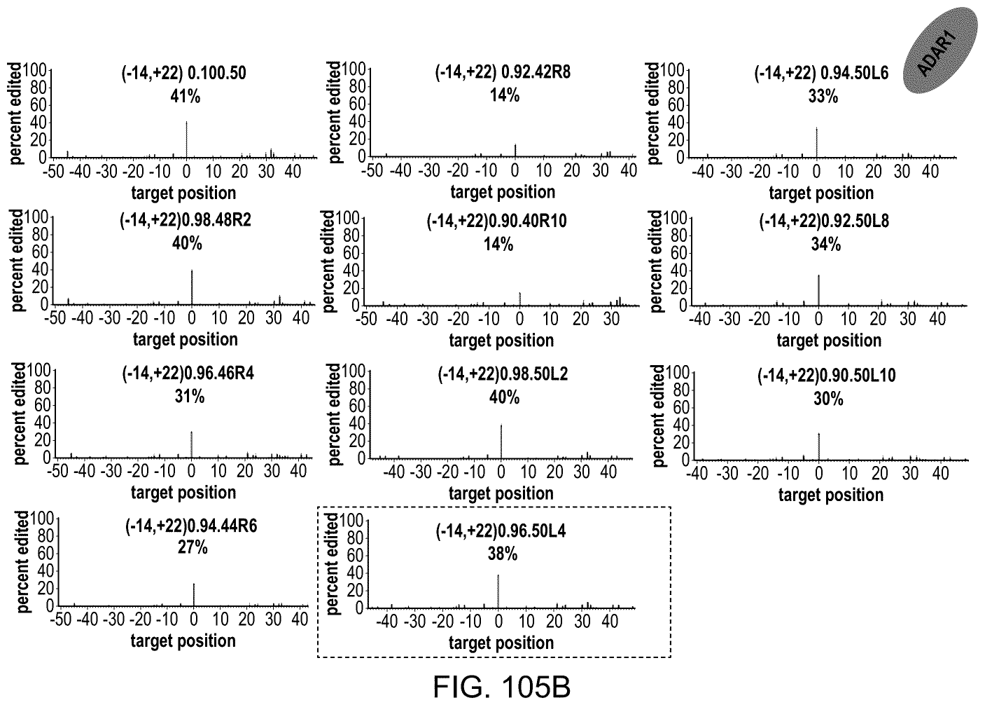

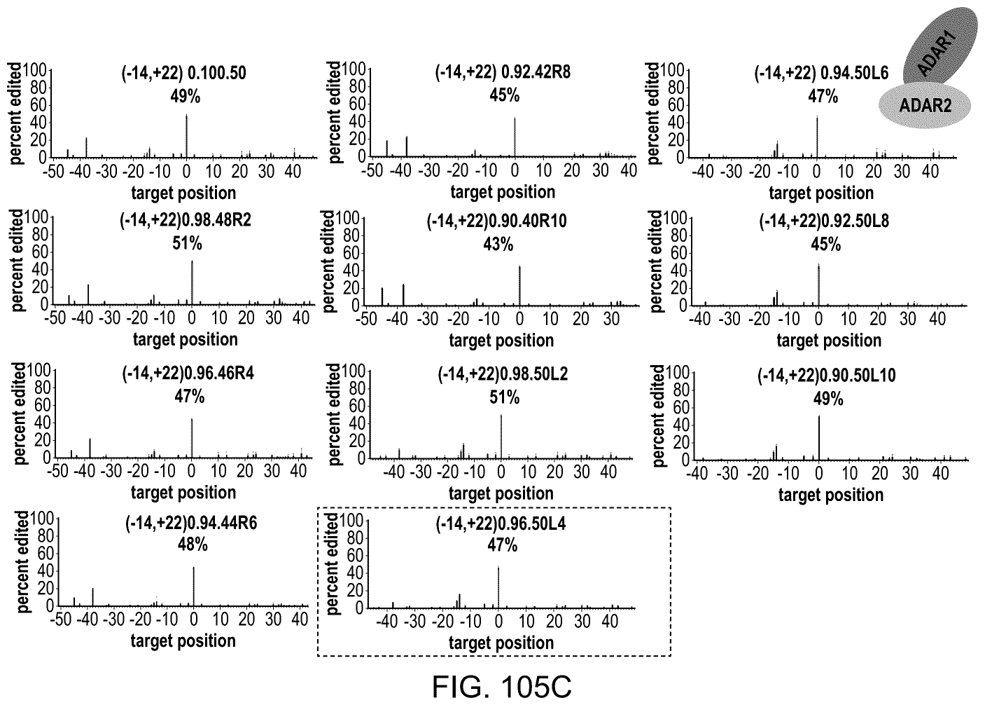

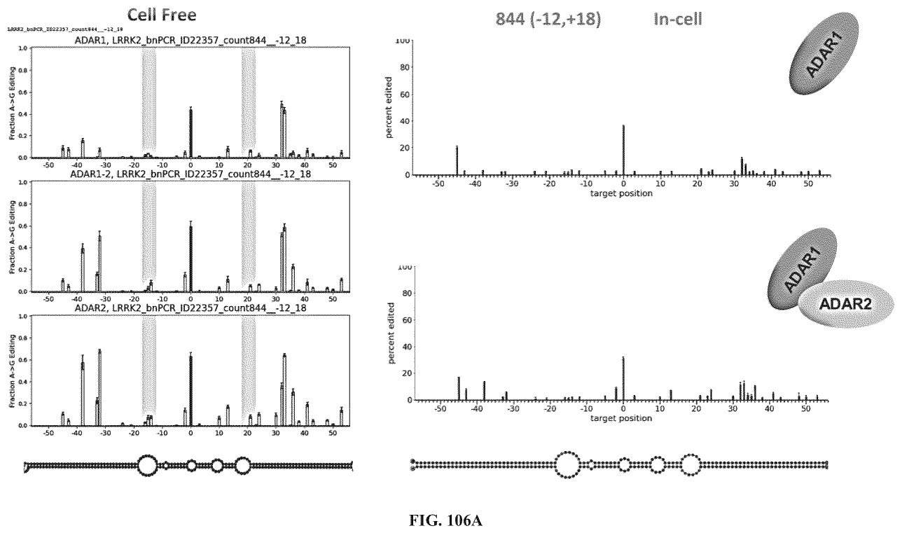

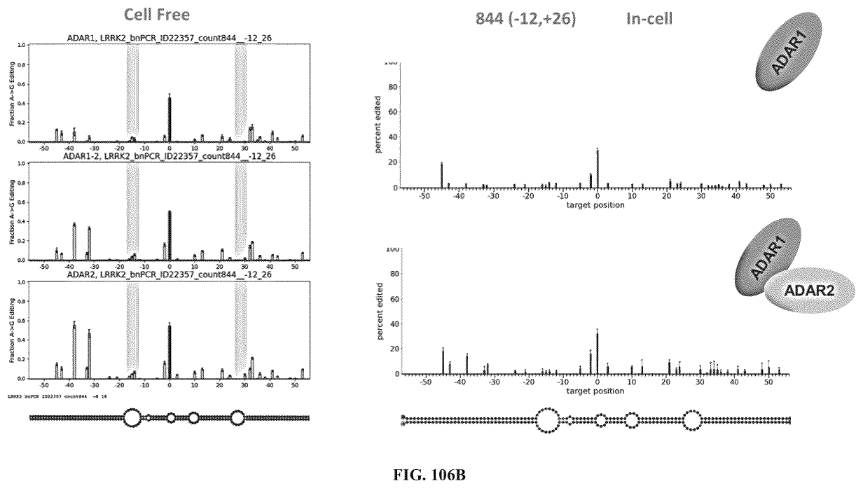

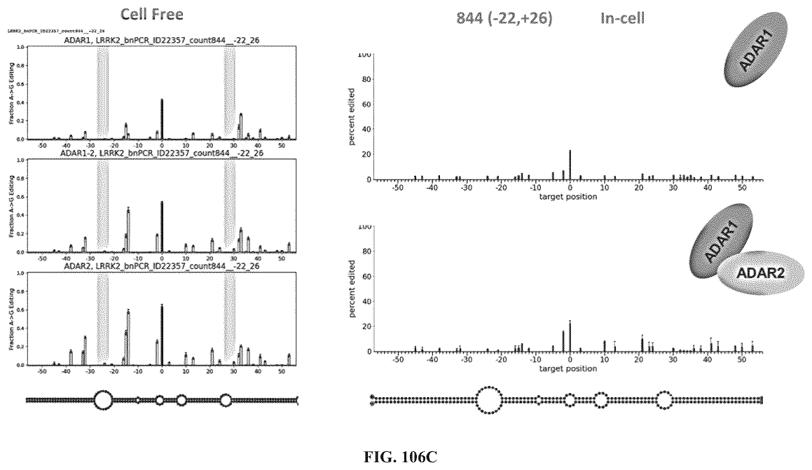

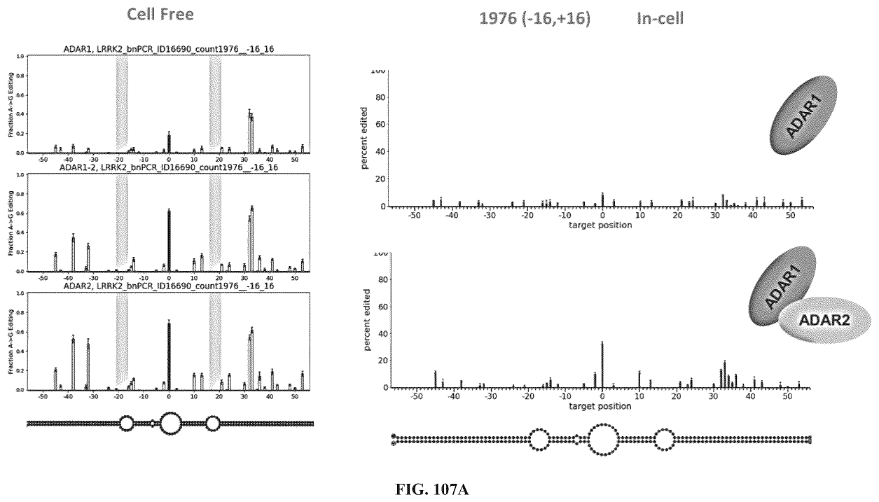

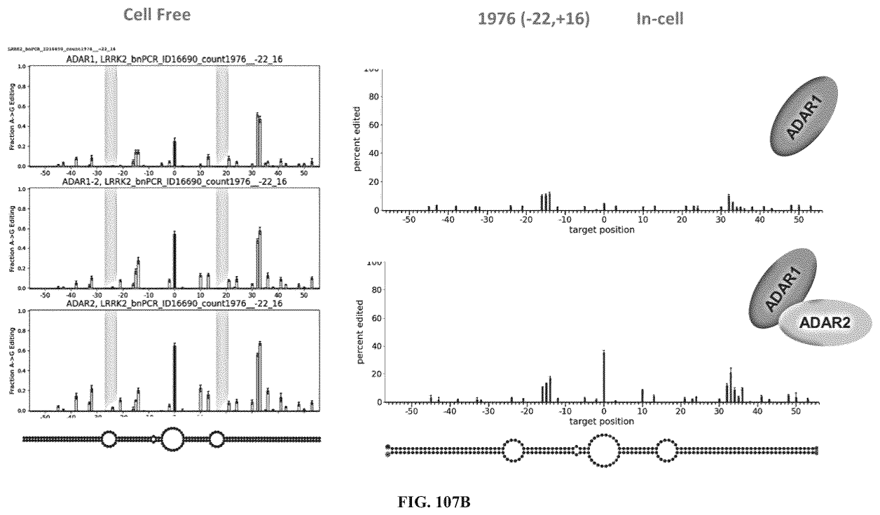

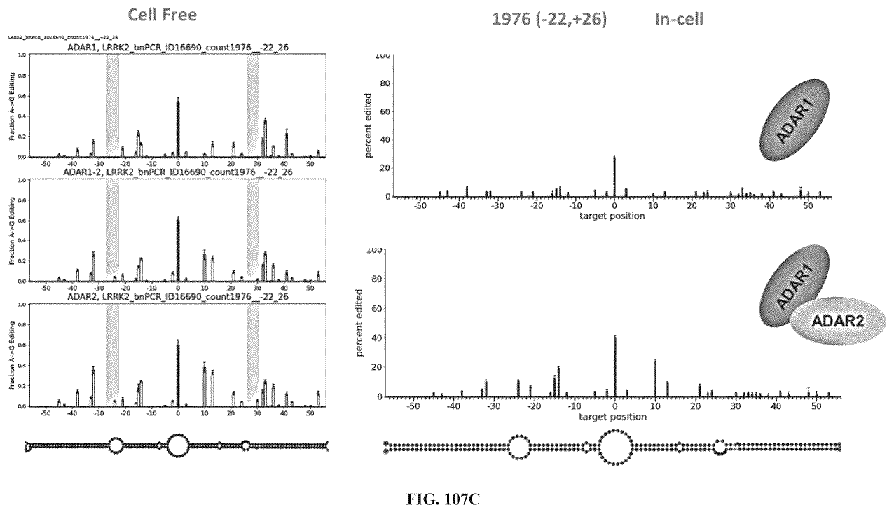

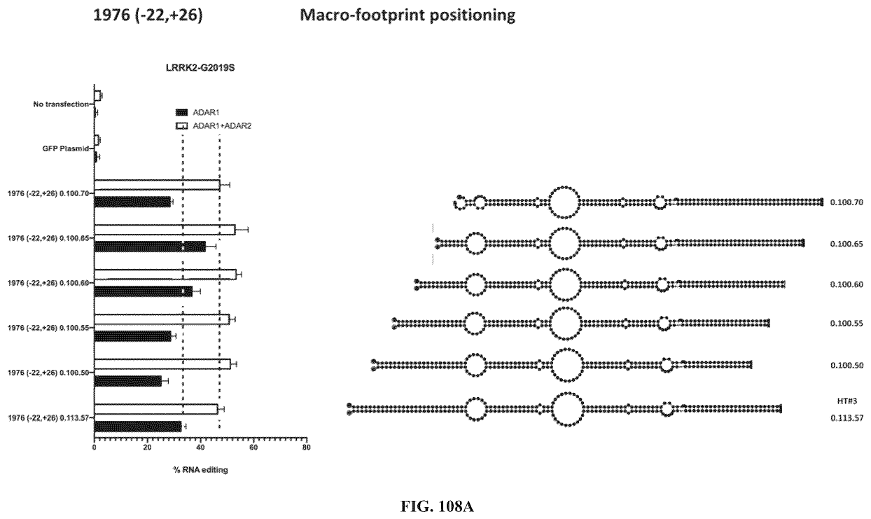

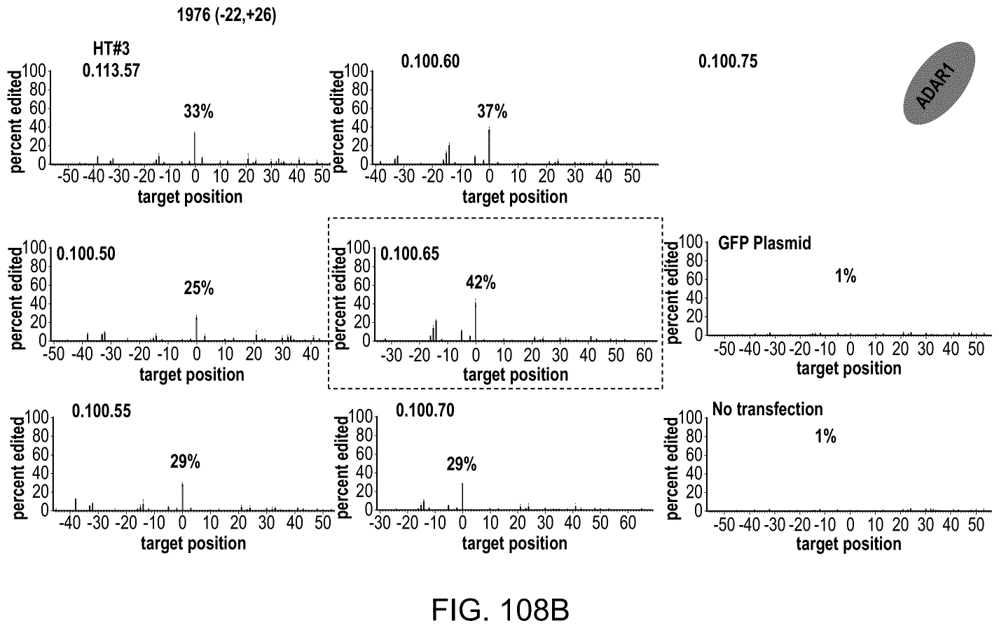

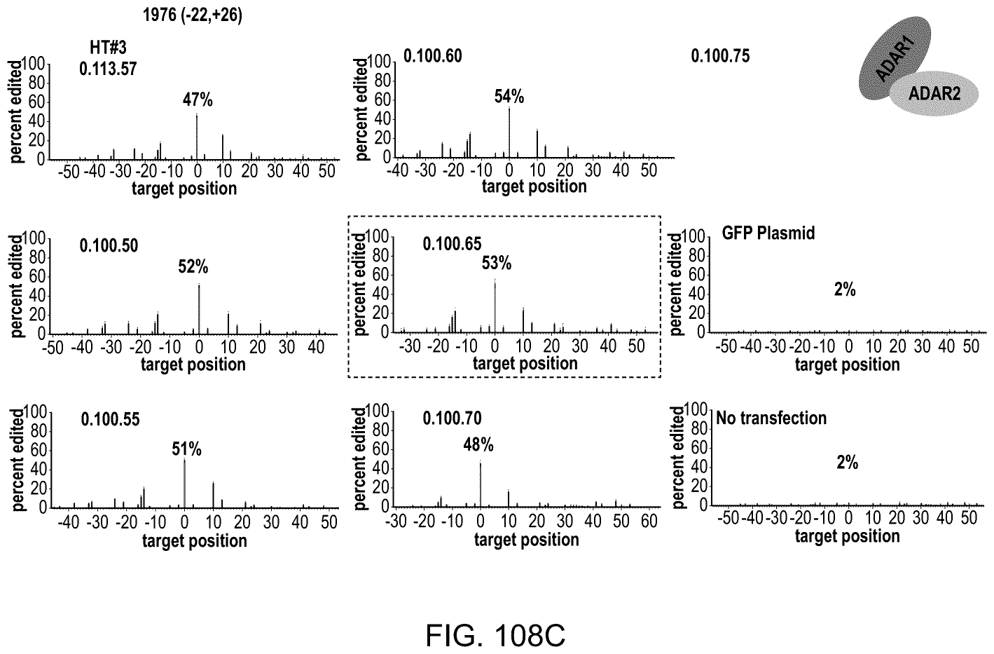

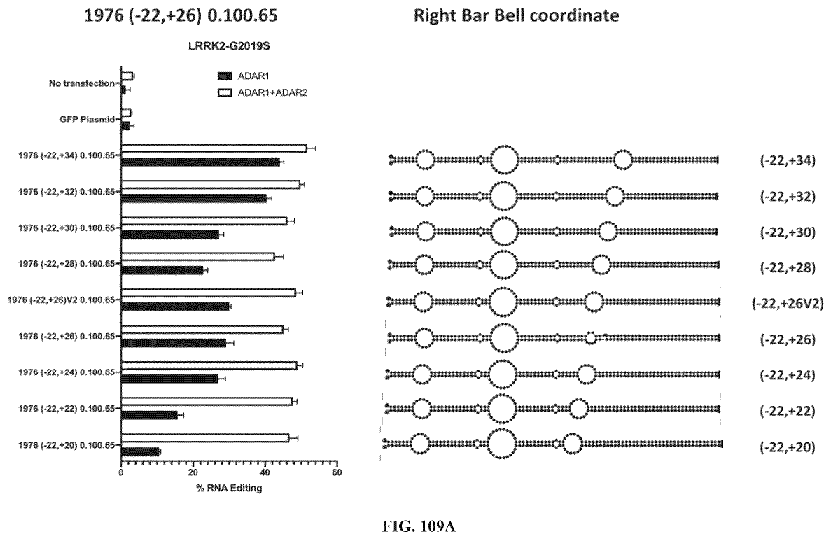

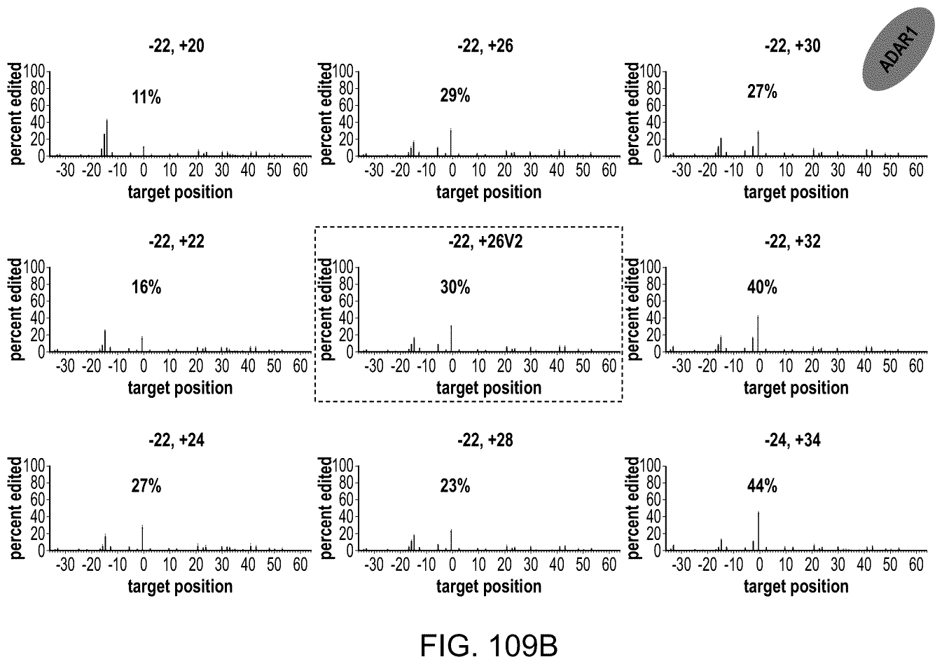

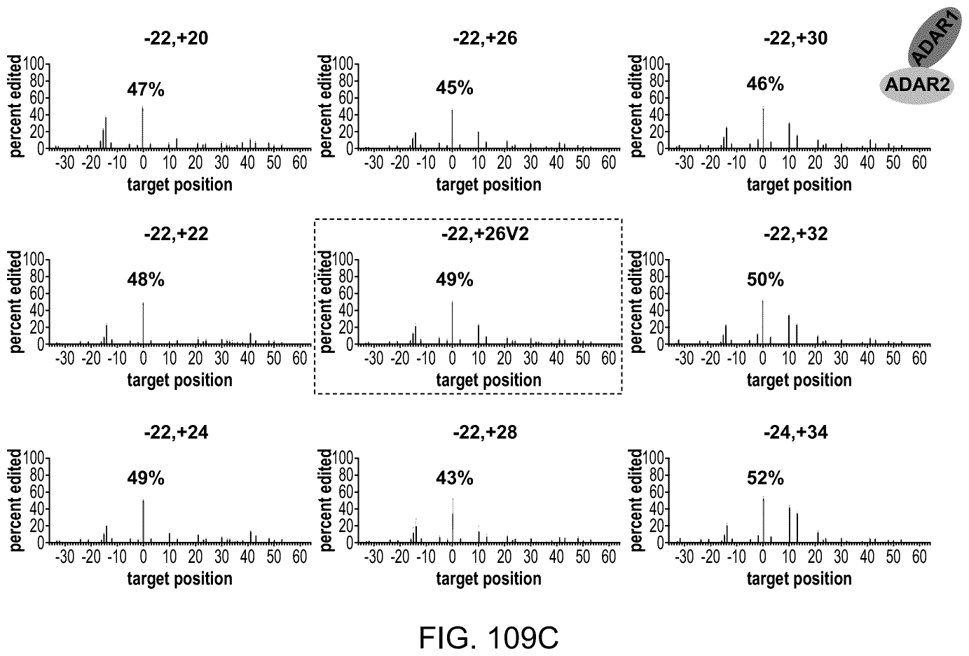

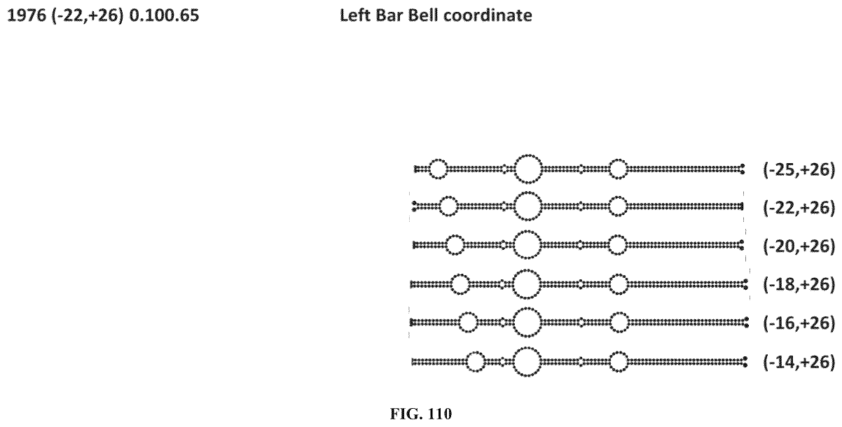

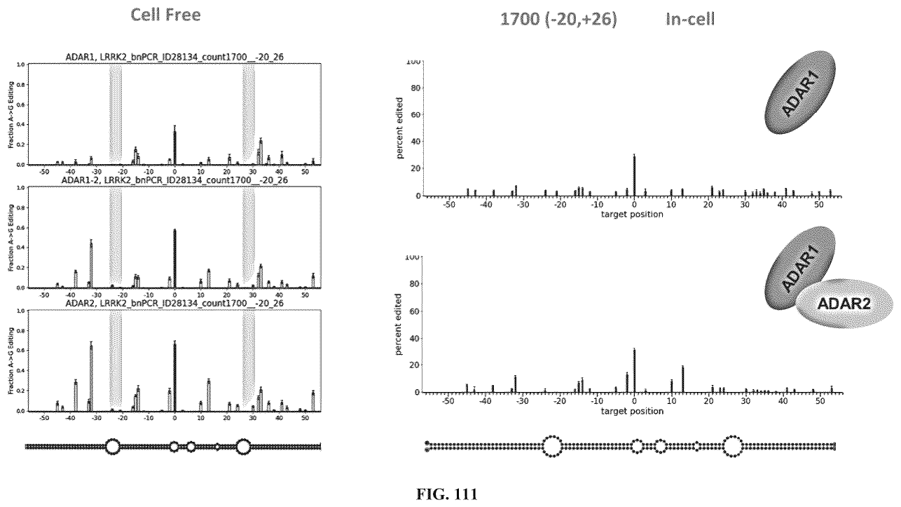

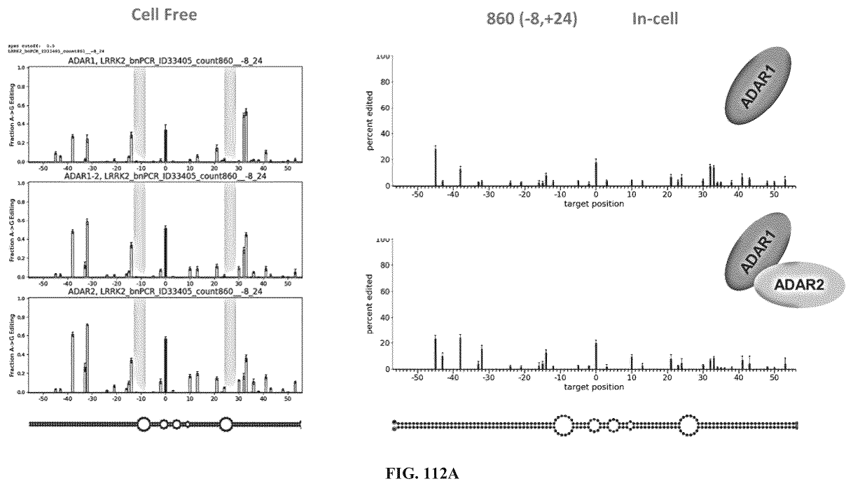

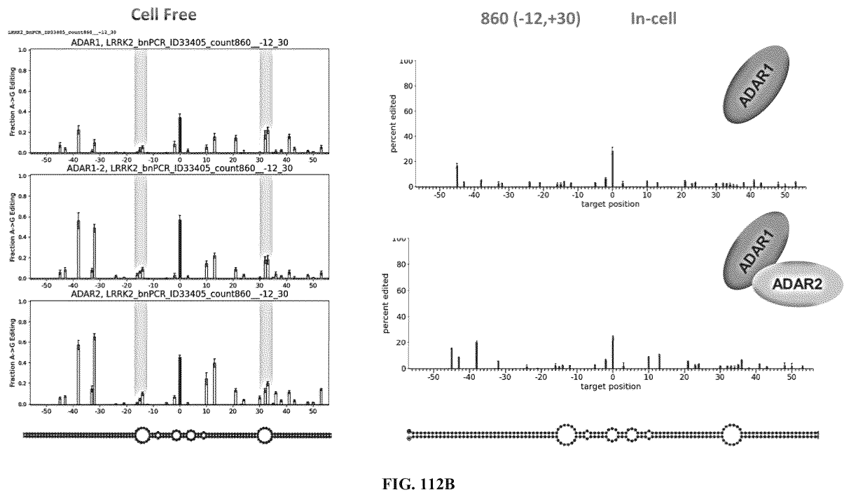

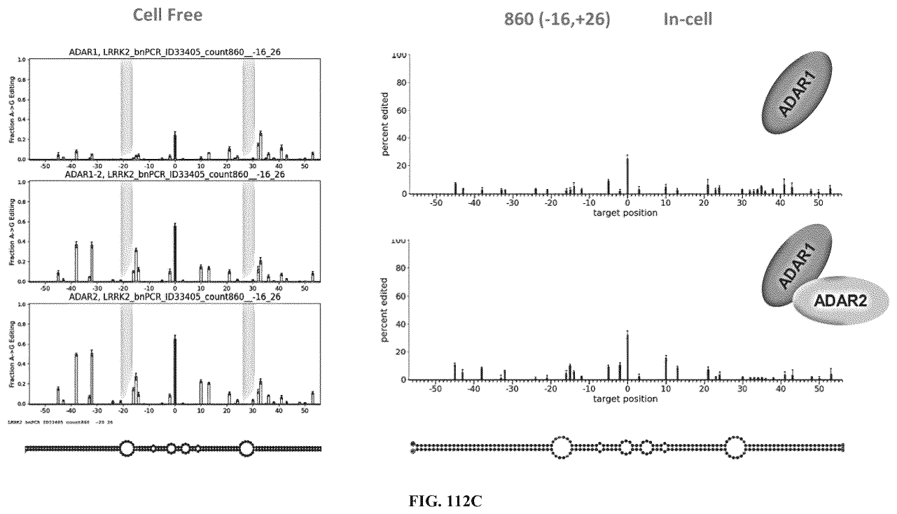

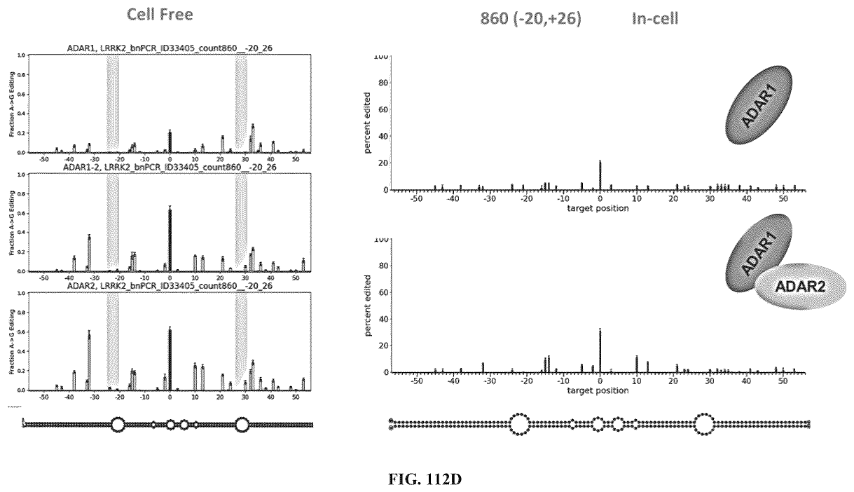

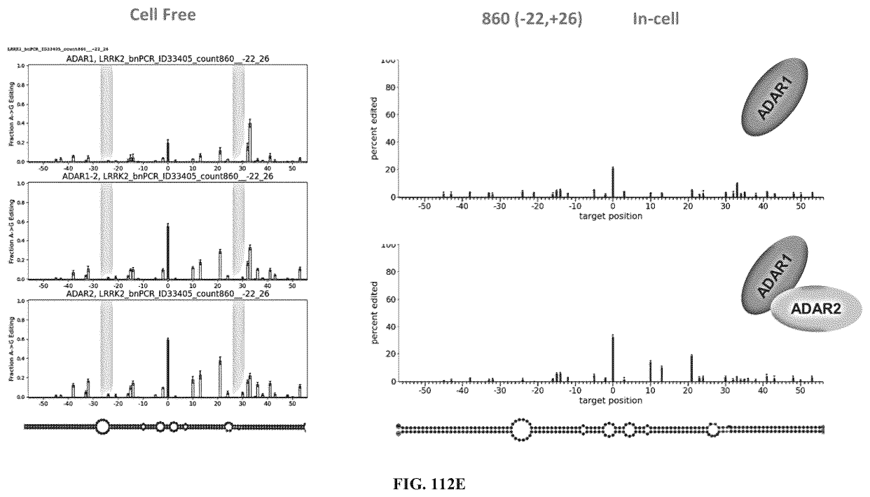

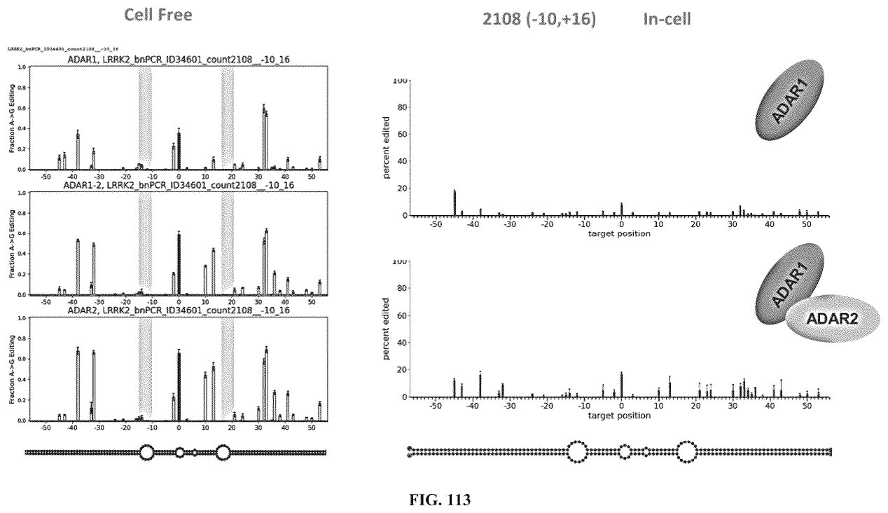

FIG. 84-FIG. 113 depict engineering of LRRK2 variants selected through high throughput screening as described above with respect to guide610. TABLE 13 below recites the sequences of the engineered guide RNAs depicted in FIGS. 84-114. While the engineered guide RNA sequences in TABLE 13 are provided as DNA sequences with a T substituted for each U, the corresponding RNA sequences are also encompassed herein.

TABLE 13
Engineered LRRK2 Guide RNA Sequences

| SEQ | | | | | Left | Right |
|---|---|---|---|---|---|---|
| ID | | | | | Barbell | Barbell |
| NO | FIG. | Guide Name | Sequence | Structural Features (target/guide) | Position | Position |
| 157 | 84A | 2063 (no loops) | CCCTGGTGTGCCCTCTGATG | −49_1-1_wobble_U-G, −8_1- | N/A | N/A |
| | | | TTTTTATCCCCATTCTACAG | 1_mismatch_C-U, −3_1-1_wobble_U-G, | | |
| | | | CAGTAGTGTGCAGTGCCGTG | 0_1-1_mismatch_A-C, 4_1- | | |
| | | | GTCATCAATCTTTGCAATGA | 1_wobble_U-G, 8_1-1_mismatch_U-U, | | |
| | | | TGGCAGCATTGGGATACAGT | 11_1-1_mismatch_G-G | | |
| | | | GTGAAGAGCAGCA | | | |
| 158 | 84B | 2063 (−14, +26) | CCCTGGTGTGCCCTCTGATG | −49_1-1_wobble_U-G, −14_6- | −14 | 26 |
| | | | TTTTTTAGGGGATTCTACAG | 6_internal_loop-symmetric_UGCAAA- | | |
| | | | CAGTAGTGTGCAGTGCCGTG | AAACGU, −8_1-1_mismatch_C-U, | | |
| | | | GTCATCAATCAAACGTATGA | −3_1-1_wobble_U-G, 0_1- | | |
| | | | TGGCAGCATTGGGATACAGT | 1_mismatch_A-C, 4_1-1_wobble_U-G, | | |
| | | | GTGAAGAGCAGCA | 8_1-1_mismatch_U-U, 11_1- | | |
| | | | | 1_mismatch_G-G, 26_6- | | |
| | | | | 6_internal_loop-symmetric_GGGGAU- | | |
| | | | | UAGGGG | | |
| 2917 | 85A | 1590 (no loops) | CCCTGGTGTGCCCTCTGATG | −49_1-1_wobble_U-G, −3_1- | N/A | 6 |
| | | | TTTTTATCCCCATTCTACAA | 1_wobble_U-G, 0_1-1_mismatch_A-C, | | |
| | | | CAGTACGGTGAAGTGCCGT | 4_1-1_wobble_U-G, 6_5- | | |
| | | | GGTCAGCAATCTTTGCAATG | 5_internal_loop-symmetric_GCUCA- | | |
| | | | ATGGCAGCATTGGGATACA | GGUGA, 17_1-1_mismatch_C-A | | |
| | | | GTGTGAAGAGCAGCA | | | |
| 2918 | 85B | 1590 (−14, +26) | CCCTGGTGTGCCCTCTGATG | −49_1-1_wobble_U-G, −14_6- | −14 | 26 |
| | | | TTTTTTAGGGGATTCTACAA | 6_internal_loop-symmetric_UGCAAA- | | |
| | | | CAGTACGGTGAAGTGCCGT | AAACGU, −3_1-1_wobble_U-G, 0_1- | | |
| | | | GGTCAGCAATCAAACGTAT | 1_mismatch_A-C, 4_1-1_wobble_U-G, | | |
| | | | GATGGCAGCATTGGGATAC | 6_5-5_internal_loop- | | |
| | | | AGTGTGAAGAGCAGCA | symmetric_GCUCA-GGUGA, 17_1- | | |
| | | | | 1_mismatch_C-A, 26_6- | | |
| | | | | 6_internal_loop-symmetric_GGGGAU- | | |
| | | | | UAGGGG | | |
| 2919 | 86A | 2397 (no loops) | CCCTGGTGTGCCCTCTGATG | −49_1-1_wobble_U-G, −3_1- | N/A | N/A |
| | | | TTTTTATCCCCATTCTACCGC | 1_wobble_U-G, 0_1-1_mismatch_A-C, | | |
| | | | TGTGCTGGGCAATCCCGTGG | 2_1-1_mismatch_C-C, 8_1- | | |
| | | | TCAGCAATCTTTGCAATGAT | 1_wobble_U-G, 12_1-1_wobble_U-G, | | |
| | | | GGCAGCATTGGGATACAGT | 15_1-1_mismatch_U-U, 18_1- | | |
| | | | GTGAAGAGCAGCA | 1_mismatch_U-C | | |
| 2920 | 86B | 2397 (−14, +26) | CCCTGGTGTGCCCTCTGATG | −49_1-1_wobble_U-G, −14 6- | −14 | 26 |
| | | | TTTTTTAGGGGATTCTACCG | 6_internal_loop-symmetric_UGCAAA- | | |
| | | | CTGTGCTGGGCAATCCCGTG | AAACGU, −3_1-1_wobble_U-G, 0_1- | | |
| | | | GTCAGCAATCAAACGTATG | 1_mismatch_A-C, 2_1-1_mismatch_C- | | |
| | | | ATGGCAGCATTGGGATACA | C, 8_1-1_wobble_U-G, 12_1- | | |
| | | | GTGTGAAGAGCAGCA | 1_wobble_U-G, 15_1-1_mismatch_U-U, | | |
| | | | | 18_1-1_mismatch_U-C, 26_6- | | |
| | | | | 6_internal_loop-symmetric_GGGGAU- | | |
| | | | | UAGGGG | | |
| 2921 | 86C | 2397 (−20, +26) | CCCTGGTGTGCCCTCTGATG | −49_1-1_wobble_U-G, −20_6- | −20 | 26 |
| | | | TTTTTTAGGGGATTCTACCG | 6_internal_loop-symmetric_CAUCAU- | | |
| | | | CTGTGCTGGGCAATCCCGTG | UACUAC, −3_1-1_wobble_U-G, 0_1- | | |
| | | | GTCAGCAATCTTTGCATACT | 1_mismatch_A-C, 2_1-1_mismatch_C- | | |
| | | | ACGCAGCATTGGGATACAG | C, 8_1-1_wobble_U-G, 12_1- | | |
| | | | TGTGAAGAGCAGCA | 1_wobble_U-G, 15_1-1_mismatch_U-U, | | |
| | | | | 18_1-1_mismatch_U-C, 26_6- | | |
| | | | | 6_internal_loop-symmetric_GGGGAU- | | |
| | | | | UAGGGG | | |
| 2922 | 87A-87C | 2397 (−14, +26) | AACTTCAGGTGCACGAAAC | -14_6-6_internal_loop- | −14 | 26 |
| | | 0.100.75 | CCTGGTGTGCCCTCTGATGT | symmetric_UGCAAA-AAACGU, −3_1- | | |
| | | | TTTTTAGGGGATTCTACCGC | 1_wobble_U-G, 0_1-1_mismatch_A-C, | | |
| | | | TGTGCTGGGCAATCCCGTGG | 2_1-1_mismatch_C-C, 8_1- | | |
| | | | TCAGCAATCAAACGTATGAT | 1_wobble_U-G, 12_1-1_wobble_U-G, | | |
| | | | G | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 2923 | 87A-87C | 2397 (−14, +26) | CAGGTGCACGAAACCCTGG | -14_6-6_internal_loop- | −14 | 26 |
| | | 0.100.70 | TGTGCCCTCTGATGTTTTTTA | symmetric_UGCAAA-AAACGU, −3_1- | | |
| | | | GGGGATTCTACCGCTGTGCT | 1_wobble_U-G, 0_1-1_mismatch_A-C, | | |
| | | | GGGCAATCCCGTGGTCAGC | 2_1-1_mismatch_C-C, 8_1- | | |
| | | | AATCAAACGTATGATGGCA | 1_wobble_U-G, 12_1-1_wobble_U-G, | | |
| | | | GC | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 2924 | 87A-87C | 2397 (−14, +26) | GCACGAAACCCTGGTGTGCC | -14_6-6_internal_loop- | −14 | 26 |
| | | 0.100.65 | CTCTGATGTTTTTTAGGGGA | symmetric_UGCAAA-AAACGU, −3_1- | | |
| | | | TTCTACCGCTGTGCTGGGCA | 1_wobble_U-G, 0_1-1_mismatch_A-C, | | |
| | | | ATCCCGTGGTCAGCAATCAA | 2_1-1_mismatch_C-C, 8_1- | | |
| | | | ACGTATGATGGCAGCATTGG | 1_wobble_U-G, 12_1-1_wobble_U-G, | | |
| | | | | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 2925 | 87A-87C | 2397 (−14, +26) | AAACCCTGGTGTGCCCTCTG | -14_6-6_internal_loop- | −14 | 26 |
| | | 0.100.60 | ATGTTTTTTAGGGGATTCTA | symmetric_UGCAAA-AAACGU, −3_1- | | |
| | | | CCGCTGTGCTGGGCAATCCC | 1_wobble_U-G, 0_1-1_mismatch_A-C, | | |
| | | | GTGGTCAGCAATCAAACGT | 2_1-1_mismatch_C-C, 8_1- | | |
| | | | ATGATGGCAGCATTGGGAT | 1_wobble_U-G, 12_1-1_wobble_U-G, | | |
| | | | AC | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 2926 | 87A-87C | 2397 (−14, +26) | CTGGTGTGCCCTCTGATGTT | -14_6-6_internal_loop- | −14 | 26 |
| | | 0.100.55 | TTTTAGGGGATTCTACCGCT | symmetric_UGCAAA-AAACGU, −3_1- | | |
| | | | GTGCTGGGCAATCCCGTGGT | 1_wobble_U-G, 0_1-1_mismatch_A-C, | | |
| | | | CAGCAATCAAACGTATGAT | 2_1-1_mismatch_C-C, 8_1- | | |
| | | | GGCAGCATTGGGATACAGT | 1_wobble_U-G, 12_1-1_wobble_U-G, | | |
| | | | GT | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 2927 | 87A-87C | 2397 (−14, +26) | GTGCCCTCTGATGTTTTTTA | -14_6-6_internal_loop- | −14 | 26 |
| | | 0.100.50 | GGGGATTCTACCGCTGTGCT | symmetric_UGCAAA-AAACGU, −3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 2928 | 87A-87C | 2397 (−14, +26) | CCCTGGTGTGCCCTCTGATG | −49_1-1_wobble_U-G, −14_6- | −14 | 26 |
| | | 0.113.57 | TTTTTTAGGGGATTCTACCG | 6_internal_loop-symmetric_UGCAAA- | | |
| | | | CTGTGCTGGGCAATCCCGTG | AAACGU, -3_1-1_wobble_U-G, 0_1- | | |
| | | | GTCAGCAATCAAACGTATG | 1_mismatch_A-C, 2_1-1_mismatch_C- | | |
| | | | ATGGCAGCATTGGGATACA | C, 8_1-1_wobble_U-G, 12_1- | | |
| | | | GTGTGAAGAGCAGCA | 1_wobble_U-G, 15_1-1_mismatch_U-U, | | |
| | | | | 18_1-1_mismatch_U-C, 26_6- | | |
| | | | | 6_internal_loop-symmetric_GGGGAU- | | |
| | | | | UAGGGG | | |
| 2929 | 88A-88C | 2397 (−14, +30) | GTGCCCTCTGATGTAAAATA | -14_6-6_internal_loop- | −14 | 30 |
| | | 0.100.50 | CCCCATTCTACCGCTGTGCT | symmetric_UGCAAA-AAACGU, -3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 30_6- | | |
| | | | | 6_internal_loop-symmetric_AUAAAA- | | |
| | | | | AAAAUA | | |
| 2930 | 88A-88C | 2397 (−14, +28) | GTGCCCTCTGATGTTTAATA | -14_6-6_internal_loop- | −14 | 28 |
| | | 0.100.50 | GGCCATTCTACCGCTGTGCT | symmetric_UGCAAA-AAACGU, -3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 2931 | 88A-88C | 2397 (−14, +26) | GTGCCCTCTGATGTTTTTTA | -14_6-6_internal_loop- | −14 | 26 |
| | | 0.100.50 | GGGGATTCTACCGCTGTGCT | symmetric_UGCAAA-AAACGU, -3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 2932 | 88A-88C | 2397 (−14, +25) | GTGCCCTCTGATGTTTTTAA | -14_6-6_internal_loop- | −14 | 25 |
| | | 0.100.50 | GGGGTTTCTACCGCTGTGCT | symmetric_UGCAAA-AAACGU, -3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 25_6- | | |
| | | | | 6_internal_loop-symmetric_UGGGGA- | | |
| | | | | AGGGGU | | |
| 2933 | 88A-88C | 2397 (−14, +23) | GTGCCCTCTGATGTTTTTAT | -14_6-6_internal_loop- | −14 | 23 |
| | | 0.100.50 | CGGGTCACTACCGCTGTGCT | symmetric_UGCAAA-AAACGU, -3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 23_6- | | |
| | | | | 6_internal_loop-symmetric_AAUGGG- | | |
| | | | | GGGUCA | | |
| 2934 | 88A-88C | 2397 (−14, +22) | GTGCCCTCTGATGTTTTTAT | -14_6-6_internal_loop- | −14 | 22 |
| | | 0.100.50 | CCGGTCAGTACCGCTGTGCT | symmetric_UGCAAA-AAACGU, -3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 22_6- | | |
| | | | | 6_internal_loop-symmetric_GAAUGG- | | |
| | | | | GGUCAG | | |
| 2935 | 88A-88C | 2397 (−14, +21) | GTGCCCTCTGATGTTTTTAT | -14_6-6_internal_loop- | −14 | 21 |
| | | 0.100.50 | CCCGTCAGAACCGCTGTGCT | symmetric_UGCAAA-AAACGU, -3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 21_6- | | |
| | | | | 6_internal_loop-symmetric_AGAAUG- | | |
| | | | | GUCAGA | | |
| 2936 | 89 | 2397 (−24, +28) | GTGCCCTCTGATGTTTAATA | -24_6-6_internal_loop- | −24 | 28 |
| | | 0.100.50 | GGCCATTCTACCGCTGTGCT | symmetric_CUGCCA-ACCGUC, -3_1- | | |
| | | | GGGCAATCCCGTGGTCAGC | 1_wobble_U-G, 0_1-1_mismatch_A-C, | | |
| | | | AATCTTTGCAATGAACCGTC | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 2937 | 89 | 2397 (−22, +28) | GTGCCCTCTGATGTTTAATA | -22_6-6_internal_loop- | −22 | 28 |
| | | 0.100.50 | GGCCATTCTACCGCTGTGCT | symmetric_GCCAUC-CUACCG, -3_1- | | |
| | | | GGGCAATCCCGTGGTCAGC | 1_wobble_U-G, 0_1-1_mismatch_A-C, | | |
| | | | AATCTTTGCAATCTACCGAG | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 2938 | 89 | 2397 (−20, +28) | GTGCCCTCTGATGTTTAATA | −20_6-6_internal_loop- | −20 | 28 |
| | | 0.100.50 | GGCCATTCTACCGCTGTGCT | symmetric_CAUCAU-UACUAC, -3_1- | | |
| | | | GGGCAATCCCGTGGTCAGC | 1_wobble_U-G, 0_1-1_mismatch_A-C, | | |
| | | | AATCTTTGCATACTACGCAG | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 2939 | 89 | 2397 (−18, +28) | GTGCCCTCTGATGTTTAATA | -18_6-6_internal_loop- | −18 | 28 |
| | | 0.100.50 | GGCCATTCTACCGCTGTGCT | symmetric_UCAUUG-GUUACU, -3_1- | | |
| | | | GGGCAATCCCGTGGTCAGC | 1_wobble_U-G, 0_1-1_mismatch_A-C, | | |
| | | | AATCTTTGGTTACTTGGCAG | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 2940 | 89 | 2397 (−16, +28) | GTGCCCTCTGATGTTTAATA | -16_6-6_internal_loop- | −16 | 28 |
| | | 0.100.50 | GGCCATTCTACCGCTGTGCT | symmetric_AUUGCA-ACGUUA, -3_1- | | |
| | | | GGGCAATCCCGTGGTCAGC | 1_wobble_U-G, 0_1-1_mismatch_A-C, | | |
| | | | AATCTTACGTTAGATGGCAG | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 2930 | 89 | 2397 (−14, +28) | GTGCCCTCTGATGTTTAATA | -14_6-6_internal_loop- | −14 | 28 |
| | | 0.100.50 | GGCCATTCTACCGCTGTGCT | symmetric_UGCAAA-AAACGU, -3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 2942 | 89 | 2397 (−12, +28) | GTGCCCTCTGATGTTTAATA | -12_6-6_internal_loop- | −12 | 28 |
| | | 0.100.50 | GGCCATTCTACCGCTGTGCT | symmetric_CAAAGA-AGAAAC, -3_1- | | |
| | | | GGGCAATCCCGTGGTCAGC | 1_wobble_U-G, 0_1-1_mismatch_A-C, | | |
| | | | AAAGAAACCAATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 2943 | 89 | 2397 (−10, +28) | GTGCCCTCTGATGTTTAATA | -10_6-6_internal_loop- | −10 | 28 |
| | | 0.100.50 | GGCCATTCTACCGCTGTGCT | symmetric_AAGAUU-UCAGAA, -3_1- | | |
| | | | GGGCAATCCCGTGGTCAGCT | 1_wobble_U-G, 0_1-1_mismatch_A-C, | | |
| | | | CAGAATGCAATGATGGCAG | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 2944 | 89 | 2397 (−9, +28) | GTGCCCTCTGATGTTTAATA | -9_6-6_internal_loop- | −9 | 28 |
| | | 0.100.50 | GGCCATTCTACCGCTGTGCT | symmetric_AGAUUG-GUCAGA, -3_1- | | |
| | | | GGGCAATCCCGTGGTCAGGT | 1_wobble_U-G, 0_1-1_mismatch_A-C, | | |
| | | | CAGATTGCAATGATGGCAG | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 2945 | 89 | 2397 (−7, +28) | GTGCCCTCTGATGTTTAATA | -7_6-6_internal_loop- | −7 | 28 |
| | | 0.100.50 | GGCCATTCTACCGCTGTGCT | symmetric_AUUGCU-UCGUCA, -3_1- | | |
| | | | GGGCAATCCCGTGGTCTCGT | 1_wobble_U-G, 0_1-1_mismatch_A-C, | | |
| | | | CACTTTGCAATGATGGCAGC | 2_1-1_mismatch_C-C, 8_1- | | |
| | | | ATTGGGATACAGTGTGAAA | 1_wobble_U-G, 12_1-1_wobble_U-G, | | |
| | | | A | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 2946 | 90A | 1321 (no loops) | CCCTGGTGTGCCCTCTGATG | −49_1-1_wobble_U-G, -7_1- | N/A | N/A |
| | | | TTTTTATCCCCATTCTAGGG | 1_wobble_U-G, -6_1-1_mismatch_G-G, | | |
| | | | CAGTAGGGTGCACTGCCGTG | -3_1-1_wobble_U-G, 0_1- | | |
| | | | GTGGGCAATCTTTGCAATGA | 1_mismatch_A-C, 4_1-1_mismatch_U- | | |
| | | | TGGCAGCATTGGGATACAGT | C, 8_1-1_mismatch_U-U, 10_2- | | |
| | | | GTGAAGAGCAGCA | 2_bulge-symmetric_AG-GG, 18_1- | | |
| | | | | 1_wobble_U-G, 19_1-1_mismatch_G-G | | |
| 2947 | 90B | 1321 (−14, +26) | CCCTGGTGTGCCCTCTGATG | −49_1-1_wobble_U-G, -14_6- | −14 | 26 |
| | | | TTTTTTAGGGGATTCTAGGG | 6_internal_loop-symmetric_UGCAAA- | | |
| | | | CAGTAGGGTGCACTGCCGTG | AAACGU, -7_1-1_wobble_U-G, -6_1- | | |
| | | | GTGGGCAATCAAACGTATG | 1_mismatch_G-G, -3_1-1_wobble_U-G, | | |
| | | | ATGGCAGCATTGGGATACA | 0_1-1_mismatch_A-C, 4_1- | | |
| | | | GTGTGAAGAGCAGCA | 1_mismatch_U-C, 8_1-1_mismatch_U- | | |
| | | | | U, 10_2-2_bulge-symmetric_AG-GG, | | |
| | | | | 18_1-1_wobble_U-G, 19_1- | | |
| | | | | 1_mismatch_G-G, 26_6- | | |
| | | | | 6_internal_loop-symmetric_GGGGAU- | | |
| | | | | UAGGGG | | |
| 2948 | 91A | 295 (no loops) | CCCTGGTGTGCCCTCTGATG | −49_1-1_wobble_U-G, -4_1- | N/A | N/A |
| | | | TTTTTATCCCCATTCTACAG | 1_mismatch_C-A, -3_1-1_wobble_U-G, | | |
| | | | CAGTCCTGTACAGTGCCGTG | 0_1-1_mismatch_A-C, 4_1- | | |
| | | | ATCAGCAATCTTTGCAATGA | 1_wobble_U-G, 7_2-2_bulge- | | |
| | | | TGGCAGCATTGGGATACAGT | symmetric_CU-UA, 12_1- | | |
| | | | GTGAAGAGCAGCA | 1_mismatch_U-C | | |
| 2949 | 91B | 295 (−14, +26) | CCCTGGTGTGCCCTCTGATG | −49_1-1_wobble_U-G, -14_6- | −14 | 26 |
| | | | TTTTTTAGGGGATTCTACAG | 6_internal_loop-symmetric_UGCAAA- | | |
| | | | CAGTCCTGTACAGTGCCGTG | AAACGU, -4_1-1_mismatch_C-A, | | |
| | | | ATCAGCAATCAAACGTATG | −3_1-1_wobble_U-G, 0_1- | | |
| | | | ATGGCAGCATTGGGATACA | 1_mismatch_A-C, 4_1-1_wobble_U-G, | | |
| | | | GTGTGAAGAGCAGCA | 7_2-2_bulge-symmetric_CU-UA, 12_1- | | |
| | | | | 1_mismatch_U-C, 26_6- | | |
| | | | | 6_internal_loop-symmetric_GGGGAU- | | |
| | | | | UAGGGG | | |
| 2950 | 92A | 730 (no loops) | CCCTGGTGTGCCCTCTGATG | −49_1-1_wobble_U-G, -3_1- | N/A | N/A |
| | | | TTTTTATCCCCCTTCTACAGC | 1_wobble_U-G, 0_1-1_mismatch_A-C, | | |
| | | | AGTAGTGAGTTTTGCCGTGG | 4_2-2_bulge-symmetric_UU-UU, 6_1- | | |
| | | | TCAGCAATCTTTGCAATGAT | 1_wobble_G-U, 11_1-1_mismatch_G-G, | | |
| | | | GGCAGCATTGGGATACAGT | 25_1-1_mismatch_U-C | | |
| | | | GTGAAGAGCAGCA | | | |
| 2951 | 92B | 730 (−14, +26) | CCCTGGTGTGCCCTCTGATG | −49_1-1_wobble_U-G, -14_6- | −14 | 25 |
| | | | TTTTTTAGGGGCTTCTACAG | 6_internal_loop-symmetric_UGCAAA- | | |
| | | | CAGTAGTGAGTTTTGCCGTG | AAACGU, -3_1-1_wobble_U-G, 0_1- | | |
| | | | GTCAGCAATCAAACGTATG | 1_mismatch_A-C, 4_2-2_bulge- | | |
| | | | ATGGCAGCATTGGGATACA | symmetric_UU-UU, 6_1-1_wobble_G- | | |
| | | | GTGTGAAGAGCAGCA | U, 11_1-1_mismatch_G-G, 25_7- | | |
| | | | | 7_internal_loop- | | |
| | | | | symmetric_UGGGGAU-UAGGGGC | | |
| 2952 | 93A | 708 (no loops) | CCCTGGTGTGCCCTCTGATG | −49_1-1_wobble_U-G, -3_1- | N/A | N/A |
| | | | TTTTTATCCCCATTCTACAG | 1_wobble_U-G, 0_1-1_mismatch_A-C, | | |
| | | | CAGTACGGTGCAGTGCCGTG | 4_1-1_wobble_U-G, 8_1- | | |
| | | | GTCAGCAATCTTTGCAATGA | 1_mismatch_U-U, 10_1-1_mismatch_A- | | |
| | | | TGGCAGCATTGGGATACAGT | G | | |
| | | | GTGAAGAGCAGCA | | | |
| 2953 | 93B | 708 (−14, +26) | CCCTGGTGTGCCCTCTGATG | −49_1-1_wobble_U-G, -14_6- | −14 | 26 |
| | | | TTTTTTAGGGGATTCTACAG | 6_internal_loop-symmetric_UGCAAA- | | |
| | | | CAGTACGGTGCAGTGCCGTG | AAACGU, -3_1-1_wobble_U-G, 0_1- | | |
| | | | GTCAGCAATCAAACGTATG | 1_mismatch_A-C, 4_1-1_wobble_U-G, | | |
| | | | ATGGCAGCATTGGGATACA | 8_1-1_mismatch_U-U, 10_1- | | |
| | | | GTGTGAAGAGCAGCA | 1_mismatch_A-G, 26_6- | | |
| | | | | 6_internal_loop-symmetric_GGGGAU- | | |
| | | | | UAGGGG | | |
| 2954 | 94A | 351 (no loops) | CCCTGGTGTGCCCTCTGATG | −49_1-1_wobble_U-G, -3_1- | N/A | N/A |
| | | | TTTTTATCCCCATTCTACAG | 1_wobble_U-G, 0_1-1_mismatch_A-C, | | |
| | | | CGGTACTGAGCAAATCCGTG | 2_2-2_bulge-symmetric_CA-AU, 15_1- | | |
| | | | GTCAGCAATCTTTGCAATGA | 1_wobble_U-G | | |
| | | | TGGCAGCATTGGGATACAGT | | | |
| | | | GTGAAGAGCAGCA | | | |
| 2955 | 94B | 351 (−14, +26) | CCCTGGTGTGCCCTCTGATG | −49_1-1_wobble_U-G, -14_6- | −14 | 26 |
| | | | TTTTTTAGGGGATTCTACAG | 6_internal_loop-symmetric_UGCAAA- | | |
| | | | CGGTACTGAGCAAATCCGTG | AAACGU, -3_1-1_wobble_U-G, 0_1- | | |
| | | | GTCAGCAATCAAACGTATG | 1_mismatch_A-C, 2_2-2_bulge- | | |
| | | | ATGGCAGCATTGGGATACA | symmetric_CA-AU, 15_1-1_wobble_U- | | |
| | | | GTGTGAAGAGCAGCA | G, 26_6-6_internal_loop- | | |
| | | | | symmetric_GGGGAU-UAGGGG | | |
| 2956 | 95A | 1326 (no loops) | CCCTGGTGTGCCCTCTGATG | −49_1-1_wobble_U-G, -4_1- | N/A | N/A |
| | | | TTTTTATCCCCATTCTACACC | 1_mismatch_C-C, 0_1-1_mismatch_A- | | |
| | | | TGGACTGAGAAATCCCGTAC | C, 2_1-1_mismatch_C-C, 6_1- | | |
| | | | TCAGCAATCTTTGCAATGAT | 1_mismatch_G-A, 13_3-3_bulge- | | |
| | | | GGCAGCATTGGGATACAGT | symmetric_ACU-UGG, 17_1- | | |
| | | | GTGAAGAGCAGCA | 1_mismatch_C-C | | |
| 2957 | 95B | 1326 (−14, +26) | CCCTGGTGTGCCCTCTGATG | −49_1-1_wobble_U-G, -14_6- | −14 | 26 |
| | | | TTTTTTAGGGGATTCTACAC | 6_internal_loop-symmetric_UGCAAA- | | |
| | | | CTGGACTGAGAAATCCCGTA | AAACGU, -4_1-1_mismatch_C-C, 0_1- | | |
| | | | CTCAGCAATCAAACGTATGA | 1_mismatch_A-C, 2_1-1_mismatch_C- | | |
| | | | TGGCAGCATTGGGATACAGT | C, 6_1-1_mismatch_G-A, 13_3- | | |
| | | | GTGAAGAGCAGCA | 3_bulge-symmetric_ACU-UGG, 17_1- | | |
| | | | | 1_mismatch_C-C, 26_6- | | |
| | | | | 6_internal_loop-symmetric_GGGGAU- | | |
| | | | | UAGGGG | | |
| 2958 | 96A-96B | 871 (−22, +26) | CCCTGGTGTGCCCTCTGATG | −49_1-1_wobble_U-G, -22_6- | −22 | 26 |
| | | | TTTTTCGAAGAATTCTACAG | 6_internal_loop-symmetric_GCCAUC- | | |
| | | | TAGGACTGAGCACTGCCGA | CAAAAA, -7_1-1_wobble_U-G, -6_1- | | |
| | | | GCTGGGCAATCTTTGCAATC | 1_mismatch_G-G, -4_0-1_bulge- | | |
| | | | AAAAAAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 26_6-6_internal_loop- | | |
| | | | | symmetric_GGGGAU-CGAAGA | | |
| 2959 | 96C-96D | 871 (−16, +22) | CCCTGGTGTGCCCTCTGATG | −49_1-1_wobble_U-G, -16_6- | −16 | 22 |
| | | | TTTTTATCCGAAACATACAG | 6_internal_loop-symmetric_AUUGCA- | | |
| | | | TAGGACTGAGCACTGCCGA | CUCCCA, -7_1-1_wobble_U-G, -6_1- | | |
| | | | GCTGGGCAATCTTCTCCCAG | 1_mismatch_G-G, -4_0-1_bulge- | | |
| | | | ATGGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 22_6-6_internal_loop- | | |
| | | | | symmetric_GAAUGG-GAAACA | | |
| 2960 | 96E-96F | 871 (−8, +26) | CCCTGGTGTGCCCTCTGATG | −49_1-1_wobble_U-G, -9_4-4_bulge- | N/A | 26 |
| | | | TTTTTTACAGAATTCTACAG | symmetric_AUUG-GUUC, -7_1- | | |
| | | | TAGGACTGAGCACTGCCGA | 1_wobble_U-G, -6_1-1_mismatch_G-G, | | |
| | | | GCTGGGGTTCCTTTGCAATG | -4_0-1_bulge-asymmetric_-C, -2_1- | | |
| | | | ATGGCAGCATTGGGATACA | 0_bulge-asymmetric_A-, 0_1- | | |
| | | | GTGTGAAGAGCAGCA | 1_mismatch_A-C, 4_1-1_mismatch_U- | | |
| | | | | C, 13_1-1_mismatch_A-G, 16_1- | | |
| | | | | 1_wobble_G-U, 26_6-6_internal_loop- | | |
| | | | | symmetric_GGGGAU-UACAGA | | |
| 139 | 96G-96I | 871 (−16, +24) | CCCTGGTGTGCCCTCTGATG | −49_1-1_wobble_U-G, -16_6- | −16 | 24 |
| | | | TTTTTATGGGGTGTCTACAG | 6_internal_loop-symmetric_AUUGCA- | | |
| | | | TAGGACTGAGCACTGCCGA | GAGUCG, -7_1-1_wobble_U-G, -6_1- | | |
| | | | GCTGGGCAATCTTGAGTCGG | 1_mismatch_G-G, -4_0-1_bulge- | | |
| | | | ATGGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 24_6-6_internal_loop- | | |
| | | | | symmetric_AUGGGG-GGGGUG | | |
| 2962 | 96J-96K | 871 (−10, +26) | CCCTGGTGTGCCCTCTGATG | −49_1-1_wobble_U-G, -10_6- | −10 | 26 |
| | | | TTTTTCATGAGATTCTACAG | 6_internal_loop-symmetric_AAGAUU- | | |
| | | | TAGGACTGAGCACTGCCGA | CUCAGG, -7_1-1_wobble_U-G, -6_1- | | |
| | | | GCTGGGCCTCAGGTGCAATG | 1_mismatch_G-G, -4_0-1_bulge- | | |
| | | | ATGGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 26_6-6_internal_loop- | | |
| | | | | symmetric_GGGGAU-CAUGAG | | |
| 2963 | 96L-96M | 871 (−16, +26) | CCCTGGTGTGCCCTCTGATG | −49_1-1_wobble_U-G, -16_6- | −16 | N/A |
| | | | TTTTTCTACAGATTCTACAG | 6_internal_loop-symmetric_AUUGCA- | | |
| | | | TAGGACTGAGCACTGCCGA | CAUUUG, -7_1-1_wobble_U-G, -6_1- | | |
| | | | GCTGGGCAATCTTCATTTGG | 1_mismatch_G-G, -4_0-1_bulge- | | |
| | | | ATGGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 26_2-2_bulge-symmetric_GG-AG, | | |
| | | | | 29_3-3_bulge-symmetric_GAU-CUA | | |
| 2964 | 96N-960 | 871 (−20, +26) | CCCTGGTGTGCCCTCTGATG | −49_1-1_wobble_U-G, −20_6- | −20 | N/A |
| | | | TTCTTTAGGGGATTCTACAG | 6_internal_loop-symmetric_CAUCAU- | | |
| | | | TAGGACTGAGCACTGCCGA | UACUAC, -7_1-1_wobble_U-G, -6_1- | | |
| | | | GCTGGGCAATCTTTGCATAC | 1_mismatch_G-G, -4_0-1_bulge- | | |
| | | | TACGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 26_5-4_internal_loop- | | |
| | | | | asymmetric_GGGGA-GGGG, 34_0- | | |
| | | | | 1_bulge-asymmetric-C | | |
| 2965 | 97A-97C | 871 (−16, +24) | AACTTCAGGTGCACGAAAC | -16_6-6_internal_loop- | −16 | 24 |
| | | 0.100.75 | CCTGGTGTGCCCTCTGATGT | symmetric_AUUGCA-GAGUCG, -7_1- | | |
| | | | TTTTATGGGGTGTCTACAGT | 1_wobble_U-G, -6_1-1_mismatch_G-G, | | |
| | | | AGGACTGAGCACTGCCGAG | -4_0-1_bulge-asymmetric_-C, -2_1- | | |
| | | | CTGGGCAATCTTGAGTCGGA | 0_bulge-asymmetric_A-, 0_1- | | |
| | | | TG | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 2966 | 97A-97C | 871 (−16, +24) | CAGGTGCACGAAACCCTGG | -16_6-6_internal_loop- | −16 | 24 |
| | | 0.100.70 | TGTGCCCTCTGATGTTTTTAT | symmetric_AUUGCA-GAGUCG, -7_1- | | |
| | | | GGGGTGTCTACAGTAGGACT | 1_wobble_U-G, -6_1-1_mismatch_G-G, | | |
| | | | GAGCACTGCCGAGCTGGGC | -4_0-1_bulge-asymmetric_-C, -2_1- | | |
| | | | AATCTTGAGTCGGATGGCAG | 0_bulge-asymmetric_A-, 0_1- | | |
| | | | C | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 2967 | 97A-97C | 871 (−16, +24) | GCACGAAACCCTGGTGTGCC | -16_6-6_internal_loop- | −16 | 24 |
| | | 0.100.65 | CTCTGATGTTTTTATGGGGT | symmetric_AUUGCA-GAGUCG, -7_1- | | |
| | | | GTCTACAGTAGGACTGAGC | 1_wobble_U-G, -6_1-1_mismatch_G-G, | | |
| | | | ACTGCCGAGCTGGGCAATCT | -4_0-1_bulge-asymmetric_-C, -2_1- | | |
| | | | TGAGTCGGATGGCAGCATTG | 0_bulge-asymmetric_A-, 0_1- | | |
| | | | G | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 2968 | 97A-97C | 871 (−16, +24) | AAACCCTGGTGTGCCCTCTG | -16_6-6_internal_loop- | −16 | 24 |
| | | 0.100.60 | ATGTTTTTATGGGGTGTCTA | symmetric_AUUGCA-GAGUCG, -7_1- | | |
| | | | CAGTAGGACTGAGCACTGC | 1_wobble_U-G, -6_1-1_mismatch_G-G, | | |
| | | | CGAGCTGGGCAATCTTGAGT | -4_0-1_bulge-asymmetric_-C, -2_1- | | |
| | | | CGGATGGCAGCATTGGGAT | 0_bulge-asymmetric_A-, 0_1- | | |
| | | | AC | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 2969 | 97A-97C | 871 (−16, +24) | CTGGTGTGCCCTCTGATGTT | -16_6-6_internal_loop- | −16 | 24 |
| | | 0.100.55 | TTTATGGGGTGTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_- C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 2970 | 97A-97C | 871 (−16, +24) | GTGCCCTCTGATGTTTTTAT | -16_6-6_internal_loop- | −16 | 24 |
| | | 0.100.50 | GGGGTGTCTACAGTAGGACT | symmetric_AUUGCA-GAGUCG, -7_1- | | |
| | | | GAGCACTGCCGAGCTGGGC | 1_wobble_U-G, -6_1-1_mismatch_G-G, | | |
| | | | AATCTTGAGTCGGATGGCAG | -4_0-1_bulge-asymmetric_-C, -2_1- | | |
| | | | CATTGGGATACAGTGTGAA | 0_bulge-asymmetric_A-, 0_1- | | |
| | | | AA | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 139 | 97A-97C | 871 (−16, +24) | CCCTGGTGTGCCCTCTGATG | −49_1-1_wobble_U-G, -16_6- | −16 | 24 |
| | | 0.113.57 | TTTTTATGGGGTGTCTACAG | 6_internal_loop-symmetric_AUUGCA- | | |
| | | | TAGGACTGAGCACTGCCGA | GAGUCG, -7_1-1_wobble_U-G, -6_1- | | |
| | | | GCTGGGCAATCTTGAGTCGG | 1_mismatch_G-G, -4_0-1_bulge- | | |
| | | | ATGGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 24_6-6_internal_loop- | | |
| | | | | symmetric_AUGGGG-GGGGUG | | |
| 2972 | 98A-98C | 871 (−16, +34) | CTGGTGTGCCCTCTGTACCA | -16_6-6_internal_loop- | −16 | 34 |
| | | 0.100.55 | ATTATCCCCATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 34_6-6_internal_loop- | | |
| | | | | symmetric_AAACAU-UACCAA | | |
| 2973 | 98A-98C | 871 (−16, +32) | CTGGTGTGCCCTCTGATCCA | -16_6-6_internal_loop- | −16 | 32 |
| | | 0.100.55 | AGCATCCCCATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2974 | 98A-98C | 871 (−16, +30) | CTGGTGTGCCCTCTGATGTA | -16_6-6_internal_loop- | −16 | 30 |
| | | 0.100.55 | AGCTACCCCATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 30_6-6_internal_loop- | | |
| | | | | symmetric_AUAAAA-AAGCUA | | |
| 2975 | 98A-98C | 871 (−16, +28) | CTGGTGTGCCCTCTGATGTT | -16_6-6_internal_loop- | −16 | 28 |
| | | 0.100.55 | TGCTAGGCCATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 28_6-6_internal_loop- | | |
| | | | | symmetric_GGAUAA-GCUAGG | | |
| 2976 | 98A-98C | 871 (−16, +26) | CTGGTGTGCCCTCTGATGTT | -16_6-6_internal_loop- | −16 | 26 |
| | | 0.100.55 | TTTTAGGGGATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 26_6-6_internal_loop- | | |
| | | | | symmetric_GGGGAU-UAGGGG | | |
| 2977 | 98A-98C | 871 (−16, +24) | CTGGTGTGCCCTCTGATGTT | -16_6-6_internal_loop- | −16 | 24 |
| | | 0.100.55 | TTTATGGGGTGTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 2978 | 98A-98C | 871 (−16, +22) | CTGGTGTGCCCTCTGATGTT | -16_6-6_internal_loop- | −16 | 22 |
| | | 0.100.55 | TTTATCCGGTGAGTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 22_6-6_internal_loop- | | |
| | | | | symmetric_GAAUGG-GGUGAG | | |
| 2979 | 98A-98C | 871 (−16, +20) | CTGGTGTGCCCTCTGATGTT | -16_6-6_internal_loop- | −16 | 20 |
| | | 0.100.55 | TTTATCCCCTGAGATCAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 20_6-6_internal_loop- | | |
| | | | | symmetric_UAGAAU-UGAGAU | | |
| 2980 | 98A-98C | 871 (−16, +18) | CTGGTGTGCCCTCTGATGTT | -16_6-6_internal_loop- | −16 | 18 |
| | | 0.100.55 | TTTATCCCCATAGATGTGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 18_6-6_internal_loop- | | |
| | | | | symmetric_UGUAGA-AGAUGU | | |
| 2981 | 99A-99C | 871 (−30, +32) | CTGGTGTGCCCTCTGATCCA | -30_6-6_internal_loop- | −30 | 32 |
| | | 0.100.55 | AGCATCCCCATTCTACAGTA | symmetric_CCAAUG-GUGACC, -7_1- | | |
| | | | GGACTGAGCACTGCCGAGC | 1_wobble_U-G, -6_1-1_mismatch_G-G, | | |
| | | | TGGGCAATCTTTGCAATGAT | -4_0-1_bulge-asymmetric_-C, -2_1- | | |
| | | | GGCAGGTGACCGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2982 | 99A-99C | 871 (−27, +32) | CTGGTGTGCCCTCTGATCCA | -27_6-6_internal_loop- | −27 | 32 |
| | | 0.100.55 | AGCATCCCCATTCTACAGTA | symmetric_AUGCUG-GUCGUG, -7_1- | | |
| | | | GGACTGAGCACTGCCGAGC | 1_wobble_U-G, -6_1-1_mismatch_G-G, | | |
| | | | TGGGCAATCTTTGCAATGAT | -4_0-1_bulge-asymmetric_-C, -2_1- | | |
| | | | GGGTCGTGTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2983 | 99A-99C | 871 (−24, +32) | CTGGTGTGCCCTCTGATCCA | -24_6-6_internal_loop- | −24 | 32 |
| | | 0.100.55 | AGCATCCCCATTCTACAGTA | symmetric_CUGCCA-ACCGUC, -7_1- | | |
| | | | GGACTGAGCACTGCCGAGC | 1_wobble_U-G, -6_1-1_mismatch_G-G, | | |
| | | | TGGGCAATCTTTGCAATGAA | -4_0-1_bulge-asymmetric_-C, -2_1- | | |
| | | | CCGTCCATTGGGATACAGTG | 0_bulge-asymmetric_A-, 0_1- | | |
| | | | T | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2984 | 99A-99C | 871 (−22, +32) | CTGGTGTGCCCTCTGATCCA | -22_6-6_internal_loop- | −22 | 32 |
| | | 0.100.55 | AGCATCCCCATTCTACAGTA | symmetric_GCCAUC-CUACCG, -7_1- | | |
| | | | GGACTGAGCACTGCCGAGC | 1_wobble_U-G, -6_1-1_mismatch_G-G, | | |
| | | | TGGGCAATCTTTGCAATCTA | -4_0-1_bulge-asymmetric_-C, -2_1- | | |
| | | | CCGAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2985 | 99A-99C | 871 (−20, +32) | CTGGTGTGCCCTCTGATCCA | −20_6-6_internal_loop- | −20 | 32 |
| | | 0.100.55 | AGCATCCCCATTCTACAGTA | symmetric_CAUCAU-CGCUAC, -7_1- | | |
| | | | GGACTGAGCACTGCCGAGC | 1_wobble_U-G, -6_1-1_mismatch_G-G, | | |
| | | | TGGGCAATCTTTGCACGCTA | -4_0-1_bulge-asymmetric_-C, -2_1- | | |
| | | | CGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2986 | 99A-99C | 871 (−18, +32) | CTGGTGTGCCCTCTGATCCA | -18_6-6_internal_loop- | −18 | 32 |
| | | 0.100.55 | AGCATCCCCATTCTACAGTA | symmetric_UCAUUG-GUCGCU, -7_1- | | |
| | | | GGACTGAGCACTGCCGAGC | 1_wobble_U-G, -6_1-1_mismatch_G-G, | | |
| | | | TGGGCAATCTTTGGTCGCTT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2987 | 99A-99C | 871 (−16, +32) | CTGGTGTGCCCTCTGATCCA | -16_6-6_internal_loop- | −16 | 32 |
| | | 0.100.55 | AGCATCCCCATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2988 | 99A-99C | 871 (−13, +32) | CTGGTGTGCCCTCTGATCCA | -13_6-6_internal_loop- | −13 | 32 |
| | | 0.100.55 | AGCATCCCCATTCTACAGTA | symmetric_GCAAAG-GAAGAG, -7_1- | | |
| | | | GGACTGAGCACTGCCGAGC | 1_wobble_U-G, -6_1-1_mismatch_G-G, | | |
| | | | TGGGCAATGAAGAGAATGA | -4_0-1_bulge-asymmetric_-C, -2_1- | | |
| | | | TGGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2989 | 100A-100C | 871 (−16, +32) | CTGGTGTGCCCTCTGATCCA | -16_6-6_internal_loop- | −16 | 32 |
| | | 0.90.55L10 | AGCATCCCCATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2 1- | | |
| | | | GGCAGCATTGG | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2990 | 100A-100C | 871 (−16, +32) | CTGGTGTGCCCTCTGATCCA | -16_6-6_internal_loop- | −16 | 32 |
| | | 0.92.55L8 | AGCATCCCCATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGA | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2991 | 100A-100C | 871 (−16, +32) | CTGGTGTGCCCTCTGATCCA | -16_6-6_internal_loop- | −16 | 32 |
| | | 0.94.55L6 | AGCATCCCCATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATA | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2992 | 100A-100C | 871 (−16, +32) | CTGGTGTGCCCTCTGATCCA | -16_6-6_internal_loop- | −16 | 32 |
| | | 0.96.55L4 | AGCATCCCCATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACA | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2993 | 100A-100C | 871 (−16, +32) | CTGGTGTGCCCTCTGATCCA | -16_6-6_internal_loop- | −16 | 32 |
| | | 0.98.55L2 | AGCATCCCCATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2994 | 100A-100C | 871 (−16, +32) | CTCTGATCCAAGCATCCCCA | -16_6-6_internal_loop- | −16 | 32 |
| | | 0.90.45R10 | TTCTACAGTAGGACTGAGCA | symmetric_AUUGCA-GAGUCG, -7_1- | | |
| | | | CTGCCGAGCTGGGCAATCTT | 1_wobble_U-G, -6_1-1_mismatch_G-G, | | |
| | | | GAGTCGGATGGCAGCATTG | -4_0-1_bulge-asymmetric_-C, -2_1- | | |
| | | | GGATACAGTGT | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2995 | 100A-100C | 871 (−16, +32) | CCCTCTGATCCAAGCATCCC | -16_6-6_internal_loop- | −16 | 32 |
| | | 0.92.47R8 | CATTCTACAGTAGGACTGAG | symmetric_AUUGCA-GAGUCG, -7_1- | | |
| | | | CACTGCCGAGCTGGGCAATC | 1_wobble_U-G, -6_1-1_mismatch_G-G, | | |
| | | | TTGAGTCGGATGGCAGCATT | -4_0-1_bulge-asymmetric_-C, -2_1- | | |
| | | | GGGATACAGTGT | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2996 | 100A-100C | 871 (−16, +32) | TGCCCTCTGATCCAAGCATC | -16_6-6_internal_loop- | −16 | 32 |
| | | 0.94.49R6 | CCCATTCTACAGTAGGACTG | symmetric_AUUGCA-GAGUCG, -7_1- | | |
| | | | AGCACTGCCGAGCTGGGCA | 1_wobble_U-G, -6_1-1_mismatch_G-G, | | |
| | | | ATCTTGAGTCGGATGGCAGC | -4_0-1_bulge-asymmetric_-C, -2_1- | | |
| | | | ATTGGGATACAGTGT | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2997 | 100A-100C | 871 (−16, +32) | TGTGCCCTCTGATCCAAGCA | -16_6-6_internal_loop- | −16 | 32 |
| | | 0.96.51R4 | TCCCCATTCTACAGTAGGAC | symmetric_AUUGCA-GAGUCG, -7_1- | | |
| | | | TGAGCACTGCCGAGCTGGG | 1_wobble_U-G, -6_1-1_mismatch_G-G, | | |
| | | | CAATCTTGAGTCGGATGGCA | -4_0-1_bulge-asymmetri_-C, -2_1- | | |
| | | | GCATTGGGATACAGTGT | 0_bulge-asymmetric_A-, 0_1- | | |
| | | | | 1_mismatch_A-C, 4_ 1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2998 | 100A-100C | 871 (−16, +32) | GGTGTGCCCTCTGATCCAAG | -16_6-6_internal_loop- | −16 | 32 |
| | | 0.98.53R2 | CATCCCCATTCTACAGTAGG | symmetric_AUUGCA-GAGUCG, -7_1- | | |
| | | | ACTGAGCACTGCCGAGCTG | 1_wobble_U-G, -6_1-1_mismatch_G-G, | | |
| | | | GGCAATCTTGAGTCGGATGG | -4_0-1_bulge-asymmetric_-C, -2_1- | | |
| | | | CAGCATTGGGATACAGTGT | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 2999 | 100A-100C | 871 (−16, +32) | CTGGTGTGCCCTCTGATCCA | -16_6-6_internal_loop- | −16 | 32 |
| | | 0.100.55 | AGCATCCCCATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 3000 | 101A, | 919 0.113.57 | CCCTGGTGTGCCCTCTGATG | −49_1-1_wobble_U-G, -22_6- | −22 | 25 |
| | 101P, | (−22, +26) | TTTTTCAGTAGCTTCTACAG | 6_internal_loop-symmetric_GCCAUC- | | |
| | 101Q, | | CAGTTCGGAGGAATCCCGA | AAGAGA, -3_1-1 wobble_U-G, -2_1- | | |
| | 101R | | GGTCAGCAATCTTTGCAATA | 1_mismatch_A-A, 0_1-1_mismatch_A- | | |
| | | | AGAGAAGCATTGGGATACA | C, 2_1-1_mismatch_C-C, 6_1- | | |
| | | | GTGTGAAGAGCAGCA | 1_mismatch_G-G, 10_3-3_bulge- | | |
| | | | | symmetric_AGU-UCG, 25_7- | | |
| | | | | 7_internal_loop- | | |
| | | | | symmetric_UGGGGAU-CAGUAGC | | |
| 3001 | 101A, | 919 0.113.57 | CCCTGGTGTGCCCTCTGATG | −49_1-1_wobble_U-G, −20_6- | −20 | 25 |
| | 101L, | (−20, +26) | TTTTTTAGGGGCTTCTACAG | 6_internal_loop-symmetric_CAUCAU- | | |
| | 101M | | CAGTTCGGAGGAATCCCGA | UACUAC, -3_1-1_wobble_U-G, -2_1- | | |
| | | | GGTCAGCAATCTTTGCATAC | 1_mismatch_A-A, 0_1-1_mismatch_A- | | |
| | | | TACGCAGCATTGGGATACA | C, 2_1-1_mismatch_C-C, 6_1- | | |
| | | | GTGTGAAGAGCAGCA | 1_mismatch_G-G, 10_3-3_bulge- | | |
| | | | | symmetric_AGU-UCG, 25_7- | | |
| | | | | 7_internal_loop- | | |
| | | | | symmetric_UGGGGAU-UAGGGGC | | |
| 3002 | 101A, | 919 0.113.57 | CCCTGGTGTGCCCTCTGATG | −49_1-1_wobble_U-G, -18_6- | −18 | 24 |
| | 101S, | (−18, +24) | TTTTTATGGAACGTCTACAG | 6_internal_loop-symmetric_UCAUUG- | | |
| | 101T | | CAGTTCGGAGGAATCCCGA | GUAGCU, -3_1-1_wobble_U-G, -2_1- | | |
| | | | GGTCAGCAATCTTTGGTAGC | 1_mismatch_A-A, 0_1-1_mismatch_A- | | |
| | | | TTGGCAGCATTGGGATACAG | C, 2_1-1_mismatch_C-C, 6_1- | | |
| | | | TGTGAAGAGCAGCA | 1_mismatch_G-G, 10_3-3_bulge- | | |
| | | | | symmetric_AGU-UCG, 24_6- | | |
| | | | | 6_internal_loop-symmetric_AUGGGG- | | |
| | | | | GGAACG | | |
| 3003 | 101A, | 919 0.113.57 | CCCTGGTGTGCCCTCTGATG | −49_1-1_wobble_U-G, -18_6- | −18 | 22 |
| | 101H, | (−18, +22) | TTTTTATCCAGCGGGTACAG | 6_internal_loop-symmetric_UCAUUG- | | |
| | 101I | | CAGTTCGGAGGAATCCCGA | GUCUUC, -3_1-1_wobble_U-G, -2_1- | | |
| | | | GGTCAGCAATCTTTGGTCTT | 1_mismatch_A-A, 0_1-1_mismatch_A- | | |
| | | | CTGGCAGCATTGGGATACA | C, 2_1-1_mismatch_C-C, 6_1- | | |
| | | | GTGTGAAGAGCAGCA | 1_mismatch_G-G, 10_3-3_bulge- | | |
| | | | | symmetric_AGU-UCG, 22_6- | | |
| | | | | 6_internal_loop-symmetric_GAAUGG- | | |
| | | | | AGCGGG | | |
| 3004 | 101A, | 919 0.113.57 | CCCTGGTGTGCCCTCTGATG | −49_1-1_wobble_U-G, -16_6- | −16 | N/A |
| | 101N, | (−16, +28) | TTTCAACGGCCCTTCTACAG | 6_internal_loop-symmetric_AUUGCA- | | |
| | 101O | | CAGTTCGGAGGAATCCCGA | ACCCUG, -3_1-1_wobble_U-G, -2_1- | | |
| | | | GGTCAGCAATCTTACCCTGG | 1_mismatch_A-A, 0_1-1_mismatch_A- | | |
| | | | ATGGCAGCATTGGGATACA | C, 2_1-1_mismatch_C-C, 6_1- | | |
| | | | GTGTGAAGAGCAGCA | 1_mismatch_G-G, 10_3-3_bulge- | | |
| | | | | symmetric_AGU-UCG, 25_1-0_bulge- | | |
| | | | | asymmetric_U-, 29_5-6_internal_loop- | | |
| | | | | asymmetric_GAUAA-CAACGG | | |
| 3005 | 101A, | 919 0.113.57 | CCCTGGTGTGCCCTCTGATG | −49_1-1_wobble_U-G, -16_6- | −16 | 24 |
| | 101J, | (−16, +24) | TTTTTATTGAGCCTCTACAG | 6_internal_loop-symmetric_AUUGCA- | | |
| | 101K | | CAGTTCGGAGGAATCCCGA | AAAUUA, -3_1-1_wobble_U-G, -2_1- | | |
| | | | GGTCAGCAATCTTAAATTAG | 1_mismatch_A-A, 0_1-1_mismatch_A- | | |
| | | | ATGGCAGCATTGGGATACA | C, 2_1-1_mismatch_C-C, 6_1- | | |
| | | | GTGTGAAGAGCAGCA | 1_mismatch_G-G, 10_3-3_bulge- | | |
| | | | | symmetric_AGU-UCG, 24_5- | | |
| | | | | 5_internal_loop-symmetric_AUGGG- | | |
| | | | | GAGCC, 29_1-1_wobble G-U | | |
| 3006 | 101A, | 919 0.113.57 | CCCTGGTGTGCCCTCTGATG | −49_1-1_wobble_U-G, -14_6- | −14 | 25 |
| | 101F, | (−14, +26) | TTTTTTAGGGGCTTCTACAG | 6_internal_loop-symmetric_UGCAAA- | | |
| | 101G | | CAGTTCGGAGGAATCCCGA | AAACGU, -3_1-1_wobble_U-G, -2_1- | | |
| | | | GGTCAGCAATCAAACGTAT | 1_mismatch_A-A, 0_1-1_mismatch_A- | | |
| | | | GATGGCAGCATTGGGATAC | C, 2_1-1_mismatch_C-C, 6_1- | | |
| | | | AGTGTGAAGAGCAGCA | 1_mismatch_G-G, 10_3-3_bulge- | | |
| | | | | symmetric_AGU-UCG, 25_7- | | |
| | | | | 7_internal_loop- | | |
| | | | | symmetric_UGGGGAU-UAGGGGC | | |
| 166 | 101A-101C | 919 0.113.57 | CCCTGGTGTGCCCTCTGATG | −49_1-1_wobble_U-G, -12_6- | −12 | 24 |
| | | (−12, +24) | TTTTTATGAGGCGTCTACAG | 6_internal_loop-symmetric_CAAAGA- | | |
| | | | CAGTTCGGAGGAATCCCGA | GACUAA, -3_1-1_wobble_U-G, -2_1- | | |
| | | | GGTCAGCAAGACTAACAAT | 1_mismatch_A-A, 0_1-1_mismatch_A- | | |
| | | | GATGGCAGCATTGGGATAC | C, 2_1-1_mismatch_C-C, 6 1- | | |
| | | | AGTGTGAAGAGCAGCA | 1_mismatch_G-G, 10_3-3_bulge- | | |
| | | | | symmetric_AGU-UCG, 24_6- | | |
| | | | | 6_internal_loop-symmetric_AUGGGG- | | |
| | | | | GAGGCG | | |
| 3008 | 101A, | 919 0.113.57 | CCCTGGTGTGCCCTCTGATG | −49_1-1_wobble_U-G, -10_6- | −10 | 24 |
| | 101D, | (−10, +24) | TTTTTATTGAGCCTCTACAG | 6_internal_loop-symmetric_AAGAUU- | | |
| | 101E | | CAGTTCGGAGGAATCCCGA | UUAGCC, -3_1-1_wobble_U-G, -2_1- | | |
| | | | GGTCAGCTTAGCCTGCAATG | 1_mismatch_A-A, 0_1-1_mismatch_A- | | |
| | | | ATGGCAGCATTGGGATACA | C, 2_1-1_mismatch_C-C, 6_1- | | |
| | | | GTGTGAAGAGCAGCA | 1_mismatch_G-G, 10_3-3_bulge- | | |
| | | | | symmetric_AGU-UCG, 24_5- | | |
| | | | | 5_internal_loop-symmetric_AUGGG- | | |
| | | | | GAGCC, 29_1-1_wobble_G-U | | |
| 3009 | 102A-102C | 919 (−12, +24) | AACTTCAGGTGCACGAAAC | -12_6-6_internal_loop- | −12 | 24 |
| | | 0.100.75 | CCTGGTGTGCCCTCTGATGT | symmetric_CAAAGA-GACUAA, -3_1- | | |
| | | | TTTTATGAGGCGTCTACAGC | 1_wobble_U-G, -2_1-1_mismatch_A-A, | | |
| | | | AGTTCGGAGGAATCCCGAG | 0_1-1_mismatch_A-C, 2_1- | | |
| | | | GTCAGCAAGACTAACAATG | 1_mismatch_C-C, 6_1-1_mismatch_G- | | |
| | | | ATG | G, 10_3-3_bulge-symmetric_AGU- | | |
| | | | | UCG, 24_6-6_internal_loop- | | |
| | | | | symmetric_AUGGGG-GAGGCG | | |
| 3010 | 102A-102C | 919 (−12, +24) | CAGGTGCACGAAACCCTGG | -12_6-6_internal_loop- | −12 | 24 |
| | | 0.100.70 | TGTGCCCTCTGATGTTTTTAT | symmetric_CAAAGA-GACUAA, -3_1- | | |
| | | | GAGGCGTCTACAGCAGTTCG | 1_wobble_U-G, -2_1-1_mismatch_A-A, | | |
| | | | GAGGAATCCCGAGGTCAGC | 0_1-1_mismatch_A-C, 2_1- | | |
| | | | AAGACTAACAATGATGGCA | 1_mismatch_C-C, 6_1-1_mismatch_G- | | |
| | | | GC | G, 10_3-3_bulge-symmetric_AGU- | | |
| | | | | UCG, 24_6-6_internal_loop- | | |
| | | | | symmetric_AUGGGG-GAGGCG | | |
| 3011 | 102A-102C | 919 (−12, +24) | GCACGAAACCCTGGTGTGCC | -12_6-6_internal_loop- | −12 | 24 |
| | | 0.100.65 | CTCTGATGTTTTTATGAGGC | symmetric_CAAAGA-GACUAA, -3_1- | | |
| | | | GTCTACAGCAGTTCGGAGG | 1_wobble_U-G, -2_1-1_mismatch_A-A, | | |
| | | | AATCCCGAGGTCAGCAAGA | 0_1-1_mismatch_A-C, 2_1- | | |
| | | | CTAACAATGATGGCAGCATT | 1_mismatch_C-C, 6_1-1_mismatch_G- | | |
| | | | GG | G, 10_3-3_bulge-symmetric_AGU- | | |
| | | | | UCG, 24_6-6_internal_loop- | | |
| | | | | symmetric_AUGGGG-GAGGCG | | |
| 3012 | 102A-102C | 919 (−12, +24) | AAACCCTGGTGTGCCCTCTG | -12_6-6_internal_loop- | −12 | 24 |
| | | 0.100.60 | ATGTTTTTATGAGGCGTCTA | symmetric_CAAAGA-GACUAA, -3_1- | | |
| | | | CAGCAGTTCGGAGGAATCC | 1_wobble_U-G, -2_1-1_mismatch_A-A, | | |
| | | | CGAGGTCAGCAAGACTAAC | 0_1-1_mismatch_A-C, 2_1- | | |
| | | | AATGATGGCAGCATTGGGA | 1_mismatch_C-C, 6_1-1_mismatch_G- | | |
| | | | TAC | G, 10_3-3_bulge-symmetric_AGU- | | |
| | | | | UCG, 24_6-6_internal_loop- | | |
| | | | | symmetric_AUGGGG-GAGGCG | | |
| 3013 | 102A-102C | 919 (−12, +24) | CTGGTGTGCCCTCTGATGTT | -12_6-6_internal_loop- | −12 | 24 |
| | | 0.100.55 | TTTATGAGGCGTCTACAGCA | symmetric_CAAAGA-GACUAA, -3_1- | | |
| | | | GTTCGGAGGAATCCCGAGG | 1_wobble_U-G, -2_1-1_mismatch_A-A, | | |
| | | | TCAGCAAGACTAACAATGA | 0_1-1_mismatch_A-C, 2_1- | | |
| | | | TGGCAGCATTGGGATACAGT | 1_mismatch_C-C, 6_1-1_mismatch_G- | | |
| | | | GT | G, 10_3-3_bulge-symmetric_AGU- | | |
| | | | | UCG, 24_6-6_internal_loop- | | |
| | | | | symmetric_AUGGGG-GAGGCG | | |
| 3014 | 102A-102C | 919 (−12, +24) | GTGCCCTCTGATGTTTTTAT | -12_6-6_internal_loop- | −12 | 24 |
| | | 0.100.50 | GAGGCGTCTACAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 24_6-6_internal_loop- | | |
| | | | | symmetric_AUGGGG-GAGGCG | | |
| 166 | 102A-102C | 919 (−12, +24) | CCCTGGTGTGCCCTCTGATG | −49_1-1_wobble_U-G, -12_6- | −12 | 24 |
| | | 0.113.57 | TTTTTATGAGGCGTCTACAG | 6_internal_loop-symmetric_CAAAGA- | | |
| | | | CAGTTCGGAGGAATCCCGA | GACUAA, -3_1-1_wobble_U-G, -2_1- | | |
| | | | GGTCAGCAAGACTAACAAT | 1_mismatch_A-A, 0_1-1_mismatch_A- | | |
| | | | GATGGCAGCATTGGGATAC | C, 2_1-1_mismatch_C-C, 6_1- | | |
| | | | AGTGTGAAGAGCAGCA | 1_mismatch_G-G, 10_3-3_bulge- | | |
| | | | | symmetric_AGU-UCG, 24_6- | | |
| | | | | 6_internal_loop-symmetric_AUGGGG- | | |
| | | | | GAGGCG | | |
| 3016 | 103A | 919 (−12, +34) | GTGCCCTCTGTACAAATTAT | -12_6-6_internal_loop- | −12 | 34 |
| | | 0.100.50 | CCCCATTCTACAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 34_6-6_internal_loop- | | |
| | | | | symmetric_AAACAU-UACAAA | | |
| 3017 | 103A | 919 (−12, +32) | GTGCCCTCTGATCAAAGCAT | -12_6-6_internal_loop- | −12 | 32 |
| | | 0.100.50 | CCCCATTCTACAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 32_6-6_internal_loop- | | |
| | | | | symmetric_AAAAAC-CAAAGC | | |
| 3018 | 103A | 919 (−12, +30) | GTGCCCTCTGATGTAAGCTA | -12_6-6_internal_loop- | −12 | 30 |
| | | 0.100.50 | CCCCATTCTACAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 30_6-6_internal_loop- | | |
| | | | | symmetric_AUAAAA-AAGCUA | | |
| 3019 | 103A | 919 (−12, +28) | GTGCCCTCTGATGTTTGCTA | -12_6-6_internal_loop- | −12 | 28 |
| | | 0.100.50 | GACCATTCTACAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 28_6-6_internal_loop- | | |
| | | | | symmetric_GGAUAA-GCUAGA | | |
| 3020 | 103A | 919 (−12, +26) | GTGCCCTCTGATGTTTTTTA | -12 6-6_internal_loop- | −12 | 26 |
| | | 0.100.50 | GAGGATTCTACAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 26_6-6_internal_loop- | | |
| | | | | symmetric_GGGGAU-UAGAGG | | |
| 3021 | 103A-103C | 919 (−12, +24) | GTGCCCTCTGATGTTTTTAT | -12_6-6_internal_loop- | −12 | 24 |
| | | 0.100.50 | GAGGCGTCTACAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 24_6-6_internal_loop- | | |
| | | | | symmetric_AUGGGG-GAGGCG | | |
| 3022 | 103A-103C | 919 (−12, +22) | GTGCCCTCTGATGTTTTTAT | -12_6-6_internal_loop- | −12 | 22 |
| | | 0.100.50 | CCGGCGAGTACAGCAGTTC | symmetric_CAAAGA-GACUAA, -3_1- | | |
| | | | GGAGGAATCCCGAGGTCAG | 1_wobble_U-G, -2_1-1_mismatch_A-A, | | |
| | | | CAAGACTAACAATGATGGC | 0_1-1_mismatch_A-C, 2_1- | | |
| | | | AGCATTGGGATACAGTGTG | 1_mismatch_C-C, 6_1-1_mismatch_G- | | |
| | | | AAAA | G, 10_3-3_bulge-symmetric_AGU- | | |
| | | | | UCG, 22_6-6_internal_loop- | | |
| | | | | symmetric_GAAUGG-GGCGAG | | |
| 3023 | 103A | 919 (−12, +20) | GTGCCCTCTGATGTTTTTAT | -12_6-6_internal_loop- | −12 | 20 |
| | | 0.100.50 | CCCCCGAGATCAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 20_6-6_internal_loop- | | |
| | | | | symmetric_UAGAAU-CGAGAU | | |
| 3024 | 103A | 919 (−12, +18) | GTGCCCTCTGATGTTTTTAT | -12_6-6_internal_loop- | −12 | 18 |
| | | 0.100.50 | CCCCATAGATGTGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 18_6-6_internal_loop- | | |
| | | | | symmetric_UGUAGA-AGAUGU | | |
| 3025 | 104A-104C | 919 (−24, +22) | GTGCCCTCTGATGTTTTTAT | -24_6-6_internal_loop- | −24 | 22 |
| | | 0.100.50 | CCGGCGAGTACAGCAGTTC | symmetric_CUGCCA-ACCGUC, -3_1- | | |
| | | | GGAGGAATCCCGAGGTCAG | 1_wobble_U-G, -2_1-1_mismatch_A-A, | | |
| | | | CAATCTTTGCAATGAACCGT | 0_1-1_mismatch_A-C, 2_1- | | |
| | | | CCATTGGGATACAGTGTGAA | 1_mismatch_C-C, 6_1-1_mismatch_G- | | |
| | | | AA | G, 10_3-3_bulge-symmetric_AGU- | | |
| | | | | UCG, 22_6-6_internal_loop- | | |
| | | | | symmetric_GAAUGG-GGCGAG | | |
| 3026 | 104A-104C | 919 (−22, +22) | GTGCCCTCTGATGTTTTTAT | -22_6-6_internal_loop- | −22 | 22 |
| | | 0.100.50 | CCGGCGAGTACAGCAGTTC | symmetric_GCCAUC-CUACCG, -3_1- | | |
| | | | GGAGGAATCCCGAGGTCAG | 1_wobble_U-G, -2_1-1_mismatch_A-A, | | |
| | | | CAATCTTTGCAATCTACCGA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 22_6-6_internal_loop- | | |
| | | | | symmetric_GAAUGG-GGCGAG | | |
| 3027 | 104A-104C | 919 (−20, +22) | GTGCCCTCTGATGTTTTTAT | −20_6-6_internal_loop- | −20 | 22 |
| | | 0.100.50 | CCGGCGAGTACAGCAGTTC | symmetric_CAUCAU-UACUAC, -3_1- | | |
| | | | GGAGGAATCCCGAGGTCAG | 1_wobble_U-G, -2_1-1_mismatch_A-A, | | |
| | | | CAATCTTTGCATACTACGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 22_6-6_internal_loop- | | |
| | | | | symmetric_GAAUGG-GGCGAG | | |
| 3028 | 104A-104C | 919 (−18, +22) | GTGCCCTCTGATGTTTTTAT | -18_6-6_internal_loop- | −18 | 22 |
| | | 0.100.50 | CCGGCGAGTACAGCAGTTC | symmetric_UCAUUG-GUUACU, -3_1- | | |
| | | | GGAGGAATCCCGAGGTCAG | 1_wobble_U-G, -2_1-1_mismatch_A-A, | | |
| | | | CAATCTTTGGTTACTTGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 22_6-6_internal_loop- | | |
| | | | | symmetric_GAAUGG-GGCGAG | | |
| 3029 | 104A-104C | 919 (−16, +22) | GTGCCCTCTGATGTTTTTAT | -16_6-6_internal_loop- | −16 | 22 |
| | | 0.100.50 | CCGGCGAGTACAGCAGTTC | symmetric_AUUGCA-AAGUUA, -3 1- | | |
| | | | GGAGGAATCCCGAGGTCAG | 1_wobble_U-G, -2_1-1_mismatch_A-A, | | |
| | | | CAATCTTAAGTTAGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 22_6-6_internal_loop- | | |
| | | | | symmetric_GAAUGG-GGCGAG | | |
| 3030 | 104A-104C | 919 (−14, +22) | GTGCCCTCTGATGTTTTTAT | -14_6-6_internal_loop- | −14 | 22 |
| | | 0.100.50 | CCGGCGAGTACAGCAGTTC | symmetric_UGCAAA-CUAAGU, -3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 22_6-6_internal_loop- | | |
| | | | | symmetric_GAAUGG-GGCGAG | | |
| 3031 | 104A-104C | 919 (−12, +22) | GTGCCCTCTGATGTTTTTAT | -12_6-6_internal_loop- | −12 | 22 |
| | | 0.100.50 | CCGGCGAGTACAGCAGTTC | symmetric_CAAAGA-GACUAA, -3_1- | | |
| | | | GGAGGAATCCCGAGGTCAG | 1_wobble_U-G, -2_1-1_mismatch_A-A, | | |
| | | | CAAGACTAACAATGATGGC | 0_1-1_mismatch_A-C, 2_1- | | |
| | | | AGCATTGGGATACAGTGTG | 1_mismatch_C-C, 6_1-1_mismatch_G- | | |
| | | | AAAA | G, 10_3-3_bulge-symmetric_AGU- | | |
| | | | | UCG, 22_6-6_internal_loop- | | |
| | | | | symmetric_GAAUGG-GGCGAG | | |
| 3032 | 104A-104C | 919 (−9, +22) | GTGCCCTCTGATGTTTTTAT | -9_6-6_internal_loop- | −9 | 22 |
| | | 0.100.50 | CCGGCGAGTACAGCAGTTC | symmetric_AGAUUG-GUUGAC, -3_1- | | |
| | | | GGAGGAATCCCGAGGTCAG | 1_wobble_U-G, -2_1-1_mismatch_A-A, | | |
| | | | GTTGACTTGCAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 22_6-6_internal_loop- | | |
| | | | | symmetric_GAAUGG-GGCGAG | | |
| 3033 | 104A-104C | 919 (−7, +22) | GTGCCCTCTGATGTTTTTAT | -7_6-6_internal_loop- | −7 | 22 |
| | | 0.100.50 | CCGGCGAGTACAGCAGTTC | symmetric_AUUGCU-UCGUUG, -3_1- | | |
| | | | GGAGGAATCCCGAGGTCTC | 1_wobble_U-G, -2_1-1_mismatch_A-A, | | |
| | | | GTTGCTTTGCAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 22_6-6_internal_loop- | | |
| | | | | symmetric_GAAUGG-GGCGAG | | |
| 3034 | 105A-105C | 919 (−14, +22) | GTGCCCTCTGATGTTTTTAT | -14_6-6_internal_loop- | −14 | 22 |
| | | 0.90.50L10 | CCGGCGAGTACAGCAGTTC | symmetric_UGCAAA-CUAAGU, -3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATAC | 1_mismatch_C-C, 6_1-1_mismatch_G- | | |
| | | | | G, 10_3-3_bulge-symmetric_AGU- | | |
| | | | | UCG, 22_6-6_internal_loop- | | |
| | | | | symmetric_GAAUGG-GGCGAG | | |
| 3035 | 105A-105C | 919 (−14, +22) | GTGCCCTCTGATGTTTTTAT | -14_6-6_internal_loop- | −14 | 22 |
| | | 0.92.50L8 | CCGGCGAGTACAGCAGTTC | symmetric_UGCAAA-CUAAGU, -3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAG | 1_mismatch_C-C, 6_1-1_mismatch_G- | | |
| | | | | G, 10_3-3_bulge-symmetric_AGU- | | |
| | | | | UCG, 22_6-6_internal_loop- | | |
| | | | | symmetric_GAAUGG-GGCGAG | | |
| 3036 | 105A-105C | 919 (−14, +22) | GTGCCCTCTGATGTTTTTAT | -14_6-6_internal_loop- | −14 | 22 |
| | | 0.94.50L6 | CCGGCGAGTACAGCAGTTC | symmetric_UGCAAA-CUAAGU, -3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAGTG | 1_mismatch_C-C, 6_1-1_mismatch_G- | | |
| | | | | G, 10_3-3_bulge-symmetric_AGU- | | |
| | | | | UCG, 22_6-6_internal_loop- | | |
| | | | | symmetric_GAAUGG-GGCGAG | | |
| 3037 | 105A-105C | 919 (−14, +22) | GTGCCCTCTGATGTTTTTAT | -14_6-6_internal_loop- | −14 | 22 |
| | | 0.96.50L4 | CCGGCGAGTACAGCAGTTC | symmetric_UGCAAA-CUAAGU, -3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAGTGTG | 1_mismatch_C-C, 6_1-1_mismatch_G- | | |
| | | | | G, 10_3-3_bulge-symmetric_AGU- | | |
| | | | | UCG, 22_6-6_internal_loop- | | |
| | | | | symmetric_GAAUGG-GGCGAG | | |
| 3038 | 105A-105C | 919 (−14, +22) | GTGCCCTCTGATGTTTTTAT | -14_6-6_internal_loop- | −14 | 22 |
| | | 0.98.50L2 | CCGGCGAGTACAGCAGTTC | symmetric_UGCAAA-CUAAGU, -3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAGTGTGA | 1_mismatch_C-C, 6_1-1_mismatch_G- | | |
| | | | A | G, 10_3-3_bulge-symmetric_AGU- | | |
| | | | | UCG, 22_6-6_internal_loop- | | |
| | | | | symmetric_GAAUGG-GGCGAG | | |
| 3039 | 105A-105C | 919 (−14, +22) | ATGTTTTTATCCGGCGAGTA | -14_6-6_internal_loop- | −14 | 22 |
| | | 0.90.40R10 | CAGCAGTTCGGAGGAATCC | symmetric_UGCAAA-CUAAGU, -3_1- | | |
| | | | CGAGGTCAGCAATCCTAAGT | 1_wobble_U-G, -2_1-1_mismatch_A-A, | | |
| | | | ATGATGGCAGCATTGGGAT | 0_1-1_mismatch_A-C, 2_1- | | |
| | | | ACAGTGTGAAAA | 1_mismatch_C-C, 6 1-1_mismatch_G- | | |
| | | | | G, 10_3-3_bulge-symmetric_AGU- | | |
| | | | | UCG, 22_6-6_internal_loop- | | |
| | | | | symmetric_GAAUGG-GGCGAG | | |
| 3040 | 105A-105C | 919 (−14, +22) | TGATGTTTTTATCCGGCGAG | -14_6-6_internal_loop- | −14 | 22 |
| | | 0.92.42R8 | TACAGCAGTTCGGAGGAAT | symmetric_UGCAAA-CUAAGU, -3_1- | | |
| | | | CCCGAGGTCAGCAATCCTAA | 1_wobble_U-G, -2_1-1_mismatch_A-A, | | |
| | | | GTATGATGGCAGCATTGGG | 0_1-1_mismatch_A-C, 2_1- | | |
| | | | ATACAGTGTGAAAA | 1_mismatch_C-C, 6_1-1_mismatch_G- | | |
| | | | | G, 10_3-3_bulge-symmetric_AGU- | | |
| | | | | UCG, 22_6-6_internal_loop- | | |
| | | | | symmetric_GAAUGG-GGCGAG | | |
| 3041 | 105A-105C | 919 (−14, +22) | TCTGATGTTTTTATCCGGCG | -14_6-6_internal_loop- | −14 | 22 |
| | | 0.94.44R6 | AGTACAGCAGTTCGGAGGA | symmetric_UGCAAA-CUAAGU, -3_1- | | |
| | | | ATCCCGAGGTCAGCAATCCT | 1_wobble_U-G, -2_1-1_mismatch_A-A, | | |
| | | | AAGTATGATGGCAGCATTG | 0_1-1_mismatch_A-C, 2_1- | | |
| | | | GGATACAGTGTGAAAA | 1_mismatch_C-C, 6_1-1_mismatch_G- | | |
| | | | | G, 10_3-3_bulge-symmetric_AGU- | | |
| | | | | UCG, 22_6-6_internal_loop- | | |
| | | | | symmetric_GAAUGG-GGCGAG | | |
| 3042 | 105A-105C | 919 (−14, +22) | CCTCTGATGTTTTTATCCGG | -14_6-6_internal_loop- | −14 | 22 |
| | | 0.96.46R4 | CGAGTACAGCAGTTCGGAG | symmetric_UGCAAA-CUAAGU, -3_1- | | |
| | | | GAATCCCGAGGTCAGCAAT | 1_wobble_U-G, -2_1-1_mismatch_A-A, | | |
| | | | CCTAAGTATGATGGCAGCAT | 0_1-1_mismatch_A-C, 2_1- | | |
| | | | TGGGATACAGTGTGAAAA | 1_mismatch_C-C, 6_1-1_mismatch_G- | | |
| | | | | G, 10_3-3_bulge-symmetric_AGU- | | |
| | | | | UCG, 22_6-6_internal_loop- | | |
| | | | | symmetric_GAAUGG-GGCGAG | | |
| 3043 | 105A-105C | 919 (−14, +22) | GCCCTCTGATGTTTTTATCC | -14_6-6_internal_loop- | −14 | 22 |
| | | 0.98.48R2 | GGCGAGTACAGCAGTTCGG | symmetric_UGCAAA-CUAAGU, -3_1- | | |
| | | | AGGAATCCCGAGGTCAGCA | 1_wobble_U-G, -2_1-1_mismatch_A-A, | | |
| | | | ATCCTAAGTATGATGGCAGC | 0_1-1_mismatch_A-C, 2_1- | | |
| | | | ATTGGGATACAGTGTGAAA | 1_mismatch_C-C, 6_1-1_mismatch_G- | | |
| | | | A | G, 10_3-3_bulge-symmetric_AGU- | | |
| | | | | UCG, 22_6-6_internal_loop- | | |
| | | | | symmetric_GAAUGG-GGCGAG | | |
| 3044 | 105A-105C | 919 (−14, +22) | GTGCCCTCTGATGTTTTTAT | -14_6-6_internal_loop- | −14 | 22 |
| | | 0.100.50 | CCGGCGAGTACAGCAGTTC | symmetric_UGCAAA-CUAAGU, -3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3-3_bulge-symmetric_AGU- | | |
| | | | | UCG, 22_6-6_internal_loop- | | |
| | | | | symmetric_GAAUGG-GGCGAG | | |
| 3045 | 106A | 844 (−12, +18) | CCCTGGTGTGCCCTCTGATG | −49_1-1_wobble_U-G, -12_8- | −12 | 18 |
| | | 0.113.57 | TTTTTATCCCCATCACCCTG | 8_internal_loop- | | |
| | | | CAGTTCATAGCAATCCCGTA | symmetric_UGCAAAGA- | | |
| | | | GTCAACAACAATATCCATGA | CAAUAUCC, -8_1-1_mismatch_C-A, | | |
| | | | TGGCAGCATTGGGATACAGT | 0_1-1_mismatch_A-C, 2_1- | | |
| | | | GTGAAGAGCAGCA | 1_mismatch_C-C, 9_2-2_bulge- | | |
| | | | | symmetric_CA-AU, 12_1- | | |
| | | | | 1_mismatch_U-U, 18_6- | | |
| | | | | 6_internal_loop-symmetric_UGUAGA- | | |
| | | | | CACCCU | | |
| 3046 | 106B | 844 (−12, +26) | CCCTGGTGTGCCCTCTGATG | −49_1-1_wobble_U-G, -12_8- | −12 | 26 |
| | | 0.113.57 | TTTTTCGGAGGATTCTACGG | 8_internal_loop- | | |
| | | | CAGTTCATAGCAATCCCGTA | symmetric_UGCAAAGA- | | |
| | | | GTCAACAAGGGTCCCCATG | GGGUCCCC, -8_1-1_mismatch_C-A, | | |
| | | | ATGGCAGCATTGGGATACA | 0_1-1_mismatch_A-C, 2_1- | | |
| | | | GTGTGAAGAGCAGCA | 1_mismatch_C-C, 9_2-2_bulge- | | |
| | | | | symmetric_CA-AU, 12_1- | | |
| | | | | 1_mismatch_U-U, 18_1-1_wobble_U-G, | | |
| | | | | 26_6-6_internal_loop- | | |
| | | | | symmetric_GGGGAU-CGGAGG | | |
| 3047 | 106C | 844 (−22, +26) | CCCTGGTGTGCCCTCTGATG | −49_1-1_wobble_U-G, -19_9- | −19 | 27 |
| | | 0.113.57 | TTTTTCTAGATATTCTACGG | 9_internal_loop- | | |
| | | | CAGTTCATAGCAATCCCGTA | symmetric_GCCAUCAUU- | | |
| | | | GTCAACAATCTTTGCCATTA | CAUUAUUUG, -8_1-1_mismatch_C- | | |
| | | | TTTGAGCATTGGGATACAGT | A, 0_1-1_mismatch_A-C, 2_1- | | |
| | | | GTGAAGAGCAGCA | 1_mismatch_C-C, 9_2-2_bulge- | | |
| | | | | symmetric_CA-AU, 12_1- | | |
| | | | | 1_mismatch_U-U, 18_1-1_wobble_U-G, | | |
| | | | | 26_1-1_wobble_G-U, 27_5- | | |
| | | | | 5_internal_loop-symmetric_GGGAU- | | |
| | | | | CUAGA | | |
| 3048 | 107A | 1976 (−16, +16) | CCCTGGTGTGCCCTCTGATG | −49_1-1_wobble_U-G, -16_6- | −16 | 16 |
| | | 0.113.57 | TTTTTATCCCCATTCGAATA | 6_internal_loop-symmetric_AUUGCA- | | |
| | | | AAGTACTGAGCTATCCCGAA | CUAUUA, -8_1-1_mismatch_C-A, -4- | | |
| | | | TTCAACAATCTTCTATTAGA | >5_10-10_internal_loop- | | |
| | | | TGGCAGCATTGGGATACAGT | symmetric_CUACAGCAUU- | | |
| | | | GTGAAGAGCAGCA | UAUCCCGAAU, 16_6- | | |
| | | | | 6_internal_loop-symmetric_GCUGUA- | | |
| | | | | GAAUAA | | |
| 3049 | 107B | 1976 (−22, +16) | CCCTGGTGTGCCCTCTGATG | −49_1-1_wobble_U-G, -22_6- | −22 | 16 |
| | | 0.113.57 | TTTTTATCCCCATTCGAAAA | 6_internal_loop-symmetric_GCCAUC- | | |
| | | | AAGTACTGAGCTATCCCGAA | CAAUCA, -8_1-1_mismatch_C-A, -4- | | |
| | | | TTCAACAATCTTTGCAATCA | >5_10-10_internal_loop- | | |
| | | | ATCAAGCATTGGGATACAGT | symmetric_CUACAGCAUU- | | |
| | | | GTGAAGAGCAGCA | UAUCCCGAAU, 16_6- | | |
| | | | | 6_internal_loop-symmetric_GCUGUA- | | |
| | | | | GAAAAA | | |
| 3050 | 107C | 1976 (−22, +26) | CCCTGGTGTGCCCTCTGATG | −49_1-1_wobble_U-G, -22_6- | −22 | N/A |
| | | 0.113.57 | TTTTTCTGGTGATTCTACAA | 6_internal_loop-symmetric_GCCAUC- | | |
| | | | CAGTACTGAGCTATCCCGAA | AAGCGA, -8_1-1_mismatch_C-A, -4- | | |
| | | | TTCAACAATCTTTGCAATAA | >5_10-10_internal_loop- | | |
| | | | GCGAAGCATTGGGATACAG | symmetric_CUACAGCAUU- | | |
| | | | TGTGAAGAGCAGCA | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 3051 | 108A-108C | 1976 (−22, +26) | CAGGTGCACGAAACCCTGG | -22_6-6_internal_loop- | −22 | N/A |
| | | 0.100.70 | TGTGCCCTCTGATGTTTTTCT | symmetric_GCCAUC-AAGCGA, -8_1- | | |
| | | | GGTGATTCTACAACAGTACT | 1_mismatch_C-A, -4→5_10- | | |
| | | | GAGCTATCCCGAATTCAACA | 10_internal_loop- | | |
| | | | ATCTTTGCAATAAGCGAAGC | symmetric_CUACAGCAUU- | | |
| | | | | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 3052 | 108A-108C | 1976 (−22, +26) | GCACGAAACCCTGGTGTGCC | -22_6-6_internal_loop- | −22 | N/A |
| | | 0.100.65 | CTCTGATGTTTTTCTGGTGA | symmetric_GCCAUC-AAGCGA, -8_1- | | |
| | | | TTCTACAACAGTACTGAGCT | 1_mismatch_C-A, -4→5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 3053 | 108A-108C | 1976 (−22, +26) | AAACCCTGGTGTGCCCTCTG | -22_6-6_internal_loop- | −22 | N/A |
| | | 0.100.60 | ATGTTTTTCTGGTGATTCTA | symmetric_GCCAUC-AAGCGA, -8_1- | | |
| | | | CAACAGTACTGAGCTATCCC | 1_mismatch_C-A, -4→5_10- | | |
| | | | GAATTCAACAATCTTTGCAA | 10_internal_loop- | | |
| | | | TAAGCGAAGCATTGGGATA | symmetric_CUACAGCAUU- | | |
| | | | C | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 3054 | 108A-108C | 1976 (−22, +26) | CTGGTGTGCCCTCTGATGTT | -22_6-6_internal_loop- | −22 | N/A |
| | | 0.100.55 | TTTCTGGTGATTCTACAACA | symmetric_GCCAUC-AAGCGA, -8_1- | | |
| | | | GTACTGAGCTATCCCGAATT | 1_mismatch_C-A, -4→5_10- | | |
| | | | CAACAATCTTTGCAATAAGC | 10_internal_loop- | | |
| | | | GAAGCATTGGGATACAGTG | symmetric_CUACAGCAUU- | | |
| | | | T | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 3055 | 108A-108C | 1976 (−22, +26) | GTGCCCTCTGATGTTTTTCT | -22_6-6_internal_loop- | −22 | N/A |
| | | 0.100.50 | GGTGATTCTACAACAGTACT | symmetric_GCCAUC-AAGCGA, -8_1- | | |
| | | | GAGCTATCCCGAATTCAACA | 1_mismatch_C-A, -4→5_10- | | |
| | | | ATCTTTGCAATAAGCGAAGC | 10_internal_loop- | | |
| | | | ATTGGGATACAGTGTGAAA | symmetric_CUACAGCAUU- | | |
| | | | A | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 3056 | 108A-108C | 1976 (−22, +26) | CCCTGGTGTGCCCTCTGATG | −49_1-1_wobble_U-G, -22_6- | −22 | N/A |
| | | 0.113.57 | TTTTTCTGGTGATTCTACAA | 6_internal_loop-symmetric_GCCAUC- | | |
| | | | CAGTACTGAGCTATCCCGAA | AAGCGA, -8_1-1_mismatch_C-A, -4- | | |
| | | | TTCAACAATCTTTGCAATAA | >5_10-10_internal_loop- | | |
| | | | GCGAAGCATTGGGATACAG | symmetric_CUACAGCAUU- | | |
| | | | TGTGAAGAGCAGCA | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 3057 | 109A-109C | 1976 (−22, +34) | GCACGAAACCCTGGTGTGCC | -22_6-6_internal_loop- | −22 | 34 |
| | | 0.100.65 | CTCTGTACAAATTATCCCCA | symmetric_GCCAUC-AAGCGA, -8_1- | | |
| | | | TTCTACAACAGTACTGAGCT | 1_mismatch_C-A, -4→5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 34_6-6_internal_loop- | | |
| | | | | symmetric_AAACAU-UACAAA | | |
| 3058 | 109A-109C | 1976 (−22, +32) | GCACGAAACCCTGGTGTGCC | -22_6-6_internal_loop- | −22 | 32 |
| | | 0.100.65 | CTCTGATCAAAAAATCCCCA | symmetric_GCCAUC-AAGCGA, -8_1- | | |
| | | | TTCTACAACAGTACTGAGCT | 1_mismatch_C-A, -4→5 10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 32_6-6_internal_loop- | | |
| | | | | symmetric_AAAAAC-CAAAAA | | |
| 3059 | 109A-109C | 1976 (−22, +30) | GCACGAAACCCTGGTGTGCC | -22_6-6_internal_loop- | −22 | 30 |
| | | 0.100.65 | CTCTGATGTAAAATGCCCCA | symmetric_GCCAUC-AAGCGA, -8_1- | | |
| | | | TTCTACAACAGTACTGAGCT | 1_mismatch_C-A, -4→5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 30_6-6_internal_loop- | | |
| | | | | symmetric_AUAAAA-AAAAUG | | |
| 3060 | 109A-109C | 1976 (−22, +28) | GCACGAAACCCTGGTGTGCC | -22_6-6_internal_loop- | −22 | 28 |
| | | 0.100.65 | CTCTGATGTTTAATGGGCCA | symmetric_GCCAUC-AAGCGA, -8_1- | | |
| | | | TTCTACAACAGTACTGAGCT | 1_mismatch_C-A, -4→5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 28_6-6_internal_loop- | | |
| | | | | symmetric_GGAUAA-AAUGGG | | |
| 3061 | 109A-109C | 1976 (−22, +26) V2 | GCACGAAACCCTGGTGTGCC | -22_6-6_internal_loop- | −22 | N/A |
| | | 0.100.65 | CTCTGATGTTTTTTGGGTGA | symmetric_GCCAUC-AAGCGA, -8_1- | | |
| | | | TTCTACAACAGTACTGAGCT | 1_mismatch_C-A, -4→5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-UGGG | | |
| 3062 | 109A-109C | 1976 (−22, +26) | GCACGAAACCCTGGTGTGCC | -22_6-6_internal_loop- | −22 | N/A |
| | | 0.100.65 | CTCTGATGTTTTTCTGGTGA | symmetric_GCCAUC-AAGCGA, -8_1- | | |
| | | | TTCTACAACAGTACTGAGCT | 1_mismatch_C-A, -4→5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 3063 | 109A-109C | 1976 (−22, +24) | GCACGAAACCCTGGTGTGCC | -22_6-6_internal_loop- | −22 | 24 |
| | | 0.100.65 | CTCTGATGTTTTTATGGTGT | symmetric_GCCAUC-AAGCGA, -8_1- | | |
| | | | ATCTACAACAGTACTGAGCT | 1_mismatch_C-A, -4→5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 24_6-6_internal_loop- | | |
| | | | | symmetric_AUGGGG-GGUGUA | | |
| 3064 | 109A-109C | 1976 (−22, +22) | GCACGAAACCCTGGTGTGCC | -22_6-6_internal_loop- | −22 | 22 |
| | | 0.100.65 | CTCTGATGTTTTTATCCGGT | symmetric_GCCAUC-AAGCGA, -8_1- | | |
| | | | AGGTACAACAGTACTGAGC | 1_mismatch_C-A, -4→5_10- | | |
| | | | TATCCCGAATTCAACAATCT | 10_internal_loop- | | |
| | | | TTGCAATAAGCGAAGCATTG | symmetric_CUACAGCAUU- | | |
| | | | G | UAUCCCGAAU, 17_1-1_mismatch_C- | | |
| | | | | A, 22_6-6_internal_loop- | | |
| | | | | symmetric_GAAUGG-GGUAGG | | |
| 3065 | 109A-109C | 1976 (−22, +20) | GCACGAAACCCTGGTGTGCC | -22_6-6_internal_loop- | −22 | 20 |
| | | 0.100.65 | CTCTGATGTTTTTATCCCCTA | symmetric_GCCAUC-AAGCGA, -8_1- | | |
| | | | GGATCAACAGTACTGAGCT | 1_mismatch_C-A, -4→5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 20_6-6_internal_loop- | | |
| | | | | symmetric_UAGAAU-UAGGAU | | |
| 3066 | 110 | 1976 (−25, +26) | GCACGAAACCCTGGTGTGCC | -25_6-6_internal_loop- | −25 | N/A |
| | | 0.100.65 | CTCTGATGTTTTTTGGGTGA | symmetric_GCUGCC-CAAUCA, -8_1- | | |
| | | | TTCTACAACAGTACTGAGCT | 1_mismatch_C-A, -4→5_10- | | |
| | | | ATCCCGAATTCAACAATCTT | 10_internal_loop- | | |
| | | | TGCAATGATCAATCAATTGG | symmetric_CUACAGCAUU- | | |
| | | | | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-UGGG | | |
| 3067 | 110 | 1976 (−22, +26) | GCACGAAACCCTGGTGTGCC | -22_6-6_internal_loop- | −22 | N/A |
| | | 0.100.65 | CTCTGATGTTTTTCTGGTGA | symmetric_GCCAUC-AAGCGA, -8_1- | | |
| | | | TTCTACAACAGTACTGAGCT | 1_mismatch_C-A, -4→5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 3068 | 110 | 1976 (−20, +26) | GCACGAAACCCTGGTGTGCC | −20_6-6_internal_loop- | −20 | N/A |
| | | 0.100.65 | CTCTGATGTTTTTTGGGTGA | symmetric_CAUCAU-CAAAGC, -8_1- | | |
| | | | TTCTACAACAGTACTGAGCT | 1_mismatch_C-A, -4→5_10- | | |
| | | | ATCCCGAATTCAACAATCTT | 10_internal_loop- | | |
| | | | TGCACAAAGCGCAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-UGGG | | |
| 3069 | 110 | 1976 (−18, +26) | GCACGAAACCCTGGTGTGCC | -18_6-6_internal_loop- | −18 | N/A |
| | | 0.100.65 | CTCTGATGTTTTTTGGGTGA | symmetric_UCAUUG-GUCAAU, -8_1- | | |
| | | | TTCTACAACAGTACTGAGCT | 1_mismatch_C-A, -4→5_10- | | |
| | | | ATCCCGAATTCAACAATCTT | 10_internal_loop- | | |
| | | | TGGTCAATTGGCAGCATTGG | symmetric_CUACAGCAUU- | | |
| | | | | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-UGGG | | |
| 3070 | 110 | 1976 (−16, +26) | GCACGAAACCCTGGTGTGCC | -16_6-6_internal_loop- | −16 | N/A |
| | | 0.100.65 | CTCTGATGTTTTTTGGGTGA | symmetric_AUUGCA-AAGUCA, -8_1- | | |
| | | | TTCTACAACAGTACTGAGCT | 1_mismatch_C-A, -4→5_10- | | |
| | | | ATCCCGAATTCAACAATCTT | 10_internal_loop- | | |
| | | | AAGTCAGATGGCAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-UGGG | | |
| 3071 | 110 | 1976 (−14, +26) | GCACGAAACCCTGGTGTGCC | -14_6-6_internal_loop- | −14 | N/A |
| | | 0.100.65 | CTCTGATGTTTTTTGGGTGA | symmetric UGCAAA-AAAAGU, -8_1- | | |
| | | | TTCTACAACAGTACTGAGCT | 1_mismatch_C-A, -4→5_10- | | |
| | | | ATCCCGAATTCAACAATCAA | 10_internal_loop- | | |
| | | | AAGTATGATGGCAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-UGGG | | |
| 3072 | 111 | 1700 (−20, +26) | CCCTGGTGTGCCCTCTGATG | −49_1-1_wobble_U-G, −20_6- | −20 | 26 |
| | | 0.113.57 | TTTTTTAGGGGATTCTACCG | 6_internal_loop-symmetric_CAUCAU- | | |
| | | | CAGTACTACCCGATCCCGTA | UACUAC, 0_1-1_mismatch_A-C, 2_1- | | |
| | | | GTCAGCAATCTTTGCATACT | 1_mismatch_C-C, 5_1-1_wobble_U-G, | | |
| | | | ACGCAGCATTGGGATACAG | 7_3-3_bulge-symmetric_CUC-ACC, | | |
| | | | TGTGAAGAGCAGCA | 18_1-1_mismatch_U-C, 26_6- | | |
| | | | | 6_internal_loop-symmetric_GGGGAU- | | |
| | | | | UAGGGG | | |
| 3073 | 112A | 860 (−8, +24) | CCCTGGTGTGCCCTCTGATG | −49_1-1_wobble_U-G, -8_6- | −8 | 24 |
| | | 0.113.57 | TTTTTATGGAAAGTCTACAG | 6_internal_loop-symmetric_GAUUGC- | | |
| | | | CAGTACAGAGGACTGCCGA | CUUUGA, -3_1-1_wobble_U-G, -2_1- | | |
| | | | GGTCACTTTGATTTGCAATG | 1_mismatch_A-A, 0_1-1_mismatch_A- | | |
| | | | ATGGCAGCATTGGGATACA | C, 4_1-1_mismatch_U-C, 6_1- | | |
| | | | GTGTGAAGAGCAGCA | 1_mismatch_G-G, 10_1-1_mismatch_A- | | |
| | | | | A, 24_6-6_internal_loop- | | |
| | | | | symmetric_AUGGGG-GGAAAG | | |
| 3074 | 112B | 860 (−12, +30) | CCCTGGTGTGCCCTCTGATG | −49_1-1_wobble_U-G, -12_6- | −12 | 30 |
| | | 0.113.57 | TATGTTCCCCCATTCTACAG | 6_internal_loop-symmetric_CAAAGA- | | |
| | | | CAGTACAGAGGACTGCCGA | CGCUAA, -8_1-1_mismatch_C-C, | | |
| | | | GGTCACCAACGCTAACAAT | −3_1-1_wobble_U-G, -2_1- | | |
| | | | GATGGCAGCATTGGGATAC | 1_mismatch_A-A, 0 1-1_mismatch_A- | | |
| | | | AGTGTGAAGAGCAGCA | C, 4_1-1_mismatch_U-C, 6_1- | | |
| | | | | 1_mismatch_G-G, 10_1-1_mismatch_A- | | |
| | | | | A, 30_6-6_internal_loop- | | |
| | | | | symmetric_AUAAAA-AUGUUC | | |
| 3075 | 112C | 860 (−16, +26) | CCCTGGTGTGCCCTCTGATG | −49_1-1_wobble_U-G, -16_6- | −16 | 26 |
| | | 0.113.57 | TTTTTCGGGAGATTCTACAG | 6_internal_loop-symmetric_AUUGCA- | | |
| | | | CAGTACAGAGGACTGCCGA | GUCUAG, -8_1-1_mismatch_C-C, | | |
| | | | GGTCACCAATCTTGTCTAGG | −3_1-1_wobble U-G, -2 1- | | |
| | | | ATGGCAGCATTGGGATACA | 1_mismatch_A-A, 0_1-1_mismatch_A- | | |
| | | | GTGTGAAGAGCAGCA | C, 4_1-1_mismatch_U-C, 6_1- | | |
| | | | | 1_mismatch_G-G, 10_1-1_mismatch_A- | | |
| | | | | A, 26_6-6_internal_loop- | | |
| | | | | symmetric_GGGGAU-CGGGAG | | |
| 3076 | 112D | 860 (−20, +26) | CCCTGGTGTGCCCTCTGATG | −49_1-1_wobble_U-G, −20_6- | −20 | 26 |
| | | 0.113.57 | TTTTTTAGGGGATTCTACAG | 6_internal_loop-symmetric_CAUCAU- | | |
| | | | CAGTACAGAGGACTGCCGA | UACUAC, -8_1-1_mismatch_C-C, | | |
| | | | GGTCACCAATCTTTGCATAC | −3_1-1_wobble_U-G, -2_1- | | |
| | | | TACGCAGCATTGGGATACA | 1_mismatch_A-A, 0_1-1_mismatch_A- | | |
| | | | GTGTGAAGAGCAGCA | C, 4_1-1_mismatch_U-C, 6_1- | | |
| | | | | 1_mismatch_G-G, 10_1-1_mismatch_A- | | |
| | | | | A, 26_6-6_internal_loop- | | |
| | | | | symmetric_GGGGAU-UAGGGG | | |
| 3077 | 112E | 860 (−22, +26) | CCCTGGTGTGCCCTCTGATG | −49_1-1_wobble_U-G, -22_6- | −22 | N/A |
| | | 0.113.57 | TTTTTCTACAGATTCTACAG | 6_internal_loop-symmetric_GCCAUC- | | |
| | | | CAGTACAGAGGACTGCCGA | AACCUG, -8_1-1_mismatch_C-C, | | |
| | | | GGTCACCAATCTTTGCAATA | −3_1-1_wobble U-G, -2_1- | | |
| | | | ACCTGAGCATTGGGATACA | 1_mismatch_A-A, 0_1-1_mismatch_A- | | |
| | | | GTGTGAAGAGCAGCA | C, 4_1-1_mismatch_U-C, 6_1- | | |
| | | | | 1_mismatch_G-G, 10_1-1_mismatch_A- | | |
| | | | | A, 26_2-2_bulge-symmetric_GG-AG, | | |
| | | | | 29_3-3_bulge-symmetric_GAU-CUA | | |
| 3078 | 113 | 2108 (−10, +16) | CCCTGGTGTGCCCTCTGATG | −49_1-1_wobble_U-G, -10_6- | −10 | 16 |
| | | 0.113.57 | TTTTTATCCCCATTCGCCTG | 6_internal_loop-symmetric_AAGAUU- | | |
| | | | AGGTACTGACCAATCCCGTA | CUAGGC, -6_1-1_wobble_G-U, 0_1- | | |
| | | | GTTAGCCTAGGCTGCAATGA | 1_mismatch_A-C, 2_1-1_mismatch_C- | | |
| | | | TGGCAGCATTGGGATACAGT | C, 7_1-1_mismatch_C-C, 15_1- | | |
| | | | GTGAAGAGCAGCA | 1_wobble_U-G, 16_6-6_internal_loop- | | |
| | | | | symmetric_GCUGUA-GCCUGA | | |
| 121 | 114 | 610 (−14, +26) | CCCTGGTGTGCCCTCTGATG | −49_1-1_wobble_U-G, -14_6- | −14 | 26 |
| | | 0.113.57 | TTTTTTAGGGGATTCTACAT | 6_internal_loop-symmetric_UGCAAA- | | |
| | | | CTGTAGTGAGCAATTCCGTG | AAACGU, -4_1-1_mismatch_C-C, | | |
| | | | CTCAGCAATCAAACGTATGA | −3_1-1_wobble_U-G, 0_1- | | |
| | | | TGGCAGCATTGGGATACAGT | 1_mismatch_A-C, 2_1-1_mismatch_C- | | |
| | | | GTGAAGAGCAGCA | U, 11_1-1_mismatch_G-G, 15_1- | | |
| | | | | 1_mismatch_U-U, 17_1-1_mismatch_C- | | |
| | | | | U, 26_6-6_internal_loop- | | |
| | | | | symmetric_GGGGAU-UAGGGG | | |
| 139 | 114 | 871 (−16, +24) | CCCTGGTGTGCCCTCTGATG | −49_1-1_wobble_U-G, -16_6- | −16 | 24 |
| | | 0.113.57 | TTTTTATGGGGTGTCTACAG | 6_internal_loop-symmetric_AUUGCA- | | |
| | | | TAGGACTGAGCACTGCCGA | GAGUCG, -7_1-1_wobble_U-G, -6_1- | | |
| | | | GCTGGGCAATCTTGAGTCGG | 1_mismatch_G-G, -4_0-1_bulge- | | |
| | | | ATGGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 24_6-6_internal_loop- | | |
| | | | | symmetric_AUGGGG-GGGGUG | | |
| 166 | 114 | 919 (−12, +24) | CCCTGGTGTGCCCTCTGATG | −49_1-1_wobble_U-G, -12_6- | −12 | 24 |
| | | 0.113.57 | TTTTTATGAGGCGTCTACAG | 6_internal_loop-symmetric_CAAAGA- | | |
| | | | CAGTTCGGAGGAATCCCGA | GACUAA, -3_1-1_wobble U-G, -2_1- | | |
| | | | GGTCAGCAAGACTAACAAT | 1_mismatch_A-A, 0_1-1_mismatch_A- | | |
| | | | GATGGCAGCATTGGGATAC | C, 2_1-1_mismatch_C-C, 6 1- | | |
| | | | AGTGTGAAGAGCAGCA | 1_mismatch_G-G, 10_3-3_bulge- | | |
| | | | | symmetric_AGU-UCG, 24_6- | | |
| | | | | 6_internal_loop-symmetric_AUGGGG- | | |
| | | | | GAGGCG | | |
| 2918 | 114 | 610 (−10, +34) | AAACCCTGGTGTGCCCTCTG | -10_6-6_internal_loop- | −10 | 34 |
| | | 0.92.60 | TCCAAATTATCCCCATTCTA | symmetric_AAGAUU-UCAGAA, -4_1- | | |
| | | | CATCTGTAGTGAGCAATTCC | 1_mismatch_C-C, -3_1-1_wobble_U-G, | | |
| | | | GTGCTCAGCTCAGAATGCAA | 0_1-1_mismatch_A-C, 2_1- | | |
| | | | TGATGGCAGCAT | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 2993 | 114 | 871 (−16, +32) | CTGGTGTGCCCTCTGATCCA | -16_6-6_internal_loop- | −16 | 32 |
| | | 0.98.55 | AGCATCCCCATTCTACAGTA | symmetric_AUUGCA-GAGUCG, -7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 3037 | 114 | 919 (−14, +22) | GTGCCCTCTGATGTTTTTAT | -14_6-6_internal_loop- | −14 | 22 |
| | | 0.96.50 | CCGGCGAGTACAGCAGTTC | symmetric_UGCAAA-CUAAGU, -3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAGTGTG | 1_mismatch_C-C, 6_1-1_mismatch_G- | | |
| | | | | G, 10_3-3_bulge-symmetric_AGU- | | |
| | | | | UCG, 22_6-6_internal_loop- | | |
| | | | | symmetric_GAAUGG-GGCGAG | | |

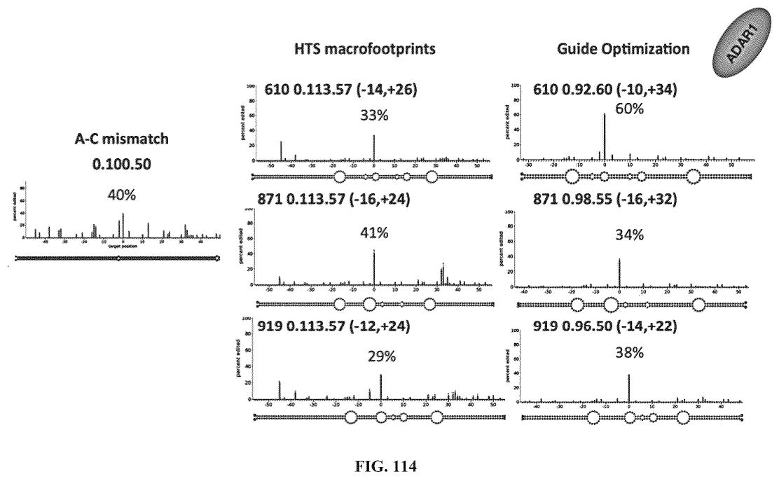

As shown in FIG. 114, engineering of guide610, guide871 and guide919 produced a significant increase in editing efficiency. Thus, this example demonstrates that guide RNAs selected via high throughput screening against LRRK2 can be systematically engineered to dramatically improve their editing efficiencies by modulating the positioning of the barbell macrofootprint within the guide-target RNA scaffold.

### Example 24

In Vitro Screening of LRRK2 gRNAs Selected Using HTS

This example describes construction of an scAAV vector for in vitro screening of LRRK2 engineered guide RNAs selected using a high throughput screen (see EXAMPLE 14) and/or engineered as described in EXAMPLE 23. For this example, count919 (−14, 22)—SEQ ID NO: 3037, count871 (−16, 32)—SEQ ID NO: 2993, count2397 (−14, 28)—SEQ ID NO: 2930, count610 (−14, 34)—SEQ ID NO: 2918 and count1976 (−22, 26)—SEQ ID NO: 3052 were evaluated.

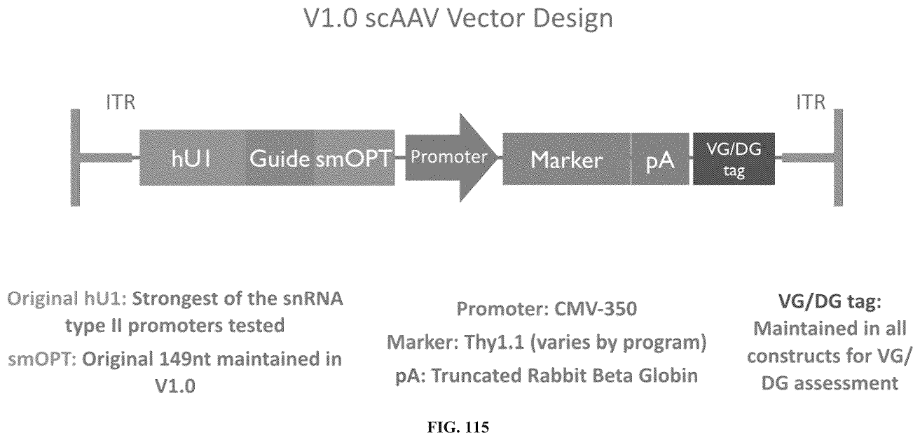

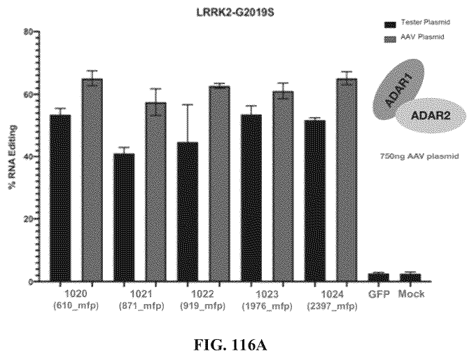

Each engineered guide RNA was cloned into an scAAV vector, as shown in FIG. 115, having a human U1 promotor (TAAGGACCAGCTTCTTTGGGAGAGAACAGACGCAGGGGCGGGAGGGAAAAAGGGAG AGGCAGACGTCACTTCCTCTTGGCGACTCTGGCAGCAGATTGGTCGGTTGAGTGGCAGA AAGGCAGACGGGGACTGGGCAAGGCACTGTCGGTGACATCACGGACAGGGCGACTTCT ATGTAGATGAGGCAGCGCAGAGGCTGCTGCTTCGCCACTTGCTGCTTCGCCACGAAGG GAGTTCCCGTGCCCTGGGAGCGGGTTCAGGACCGCTGATCGGAAGTGAGAATCCCAGC TGTGTGTCAGGGCTGGAAAGGGCTCGGGAGTGCGCGGGGCAAGTGACCGTGTGTGTAA AGAGTGAGGCGTATGAGGCTGTGTCGGGGCAGAGCCCGAAGATCTC)—SEQ ID NO: 3079 and an SmOPT sequence (AATTTTTGGAG)—SEQ ID NO: 3080 flanking each guide. Each vector was transfected into HEK293 cells, and the percent RNA editing facilitated by each engineered guide RNA via ADAR1+ADAR2 was compared to control. As shown in FIG. 116A, each engineered guide RNA transfected facilitated higher levels of editing relative to the control. Each variant was then packaged into an scAAV virus, and the ability of each guide to facilitate editing via ADAR1+ADAR2 after transduction was determined. As shown in FIG. 116B, each guide RNA displayed comparable editing when packaged as an scAAV virus via transduction as when transfected as an AAV plasmid. Following differentiation, all cell lines selected as LRRK2 in vitro models display key features of neuronal development including neurite outgrowth and cell-to-cell connections. As such, this example demonstrates repair of neuronal development in the in vitro cell model upon transfection with the scAAV vector containing engineered guide RNAs.

### Example 25

### Editing Specificity of Exemplary Engineered Guide RNAs Targeting ABCA4 G5882A Mutation (G1961E Amino Acid Mutation)

Exemplary engineered guide RNAs were screened for facilitating editing of the G5882A point mutation (G1961E amino acid mutation) in ABCA4 mRNA. Each engineered guide RNA contains latent structure that results in a micro-footprint and macro-footprint within the guide-target RNA scaffold, upon hybridization of the engineered guide RNA to the target RNA.

All polynucleotide sequences encoding for the engineered guide RNA of TABLE 14, are also encompassed herein. Further, while the engineered guide RNA sequences in TABLE 14 are provided as DNA sequences with a T substituted for each U, the corresponding RNA sequences are also encompassed herein. For reference, each structural feature is annotated as follows: position of the structural feature with a negative value indicating upstream of the target A and a positive value indicating downstream of the target A, the name of the structural feature (e.g., symmetric bulge, symmetric internal loop, asymmetric bulge, asymmetric internal loop, or mismatch), and the sequence of participating bases on the target RNA side and the guide RNA side. TABLE 14 further includes the amount of on target editing achieved via ADAR1 or ADAR2 separately, as well as ADAR1 and ADAR2. The specificity of each guide was also calculated for each engineered guide via ADAR1, ADAR2, and ADAR1+ADAR2. Specificity as provided in TABLE 14 was calculated using the formula: Specificity=(fraction on-target editing+1)/(sum(non-synonymous off-target fraction editing)). These data highlight the diverse sequence space represented by the ABCA4-targeting engineered guide RNAs of the present disclosure, which have a range of different structural features that drive sequence diversity and which exhibit high on-target editing efficiency. FIG. 117A-FIG. 117B illustrate editing of ABCA4 mRNA using exemplary guides of TABLE 14 via ADAR1.

TABLE 14
Engineered Guide RNAs with Barbells Targeting ABCA4

| SEQ | | | | | | ADAR2 | |
|---|---|---|---|---|---|---|---|
| ID | | | Engineered Guide RNAs with | ADAR1 | ADAR1 | on- | ADAR2 |
| NO | Guide Name | Structural Features (target/guide) | Barbells Targeting ABCA4 | on-target | specificity | target | specificity |
| 2729 | ABCA4-GA- | −6_6-6_internal_loop-symmetric_GCUGUG- | ATCTTGAATGTGGTTGTTTTGCCG | 0.486 | 1.442 | 0.116 | 1.077 |
| | GC_−6_30 | AAAAUG; −1->0_2-2_bulge-symmetric_GA- | GCACCATTCAACAGTTGGAGGCC | | | | |
| | | CG; 30_6-6_internal_loop- | AAAGCACTCTCCAGGGCGAACTC | | | | |
| | | symmetric_UGGGAG-ACAGUU | GGACAAAAATGCTGTCCACTGCTG | | | | |
| | | | GGCTGG | | | | |
| 2730 | ABCA4-GA- | −16_6-6_internal_loop-symmetric_CAGUGG- | ATCTTGAATGTGGTTGTTTTGCCG | 0.419 | 1.318 | 0.189 | 1.118 |
| | GC_−16_18 | GAUUAA; −1->0_2-2_bulge-symmetric_GA- | GCACCATTCACTCCCAGGAGGCGC | | | | |
| | | CG; 18_6-6_internal_loop- | ACTAACTCTCCAGGGCGAACTCGG | | | | |
| | | symmetric_GCUUUG-GCACUA | ACACACAGCCTGTGATTAACTGGG | | | | |
| | | | CTGG | | | | |
| 2731 | ABCA4-GA- | −10_6-6_internal_loop-symmetric_ACAGGC- | ATCTTGAATGTGGTTGTTTTGCCG | 0.319 | 1.286 | 0.152 | 1.113 |
| | GC_−10_26 | AGACAG; −1->0_2-2_bulge-symmetric_GA- | GCACCATTCACTCCAATCACGCCA | | | | |
| | | CG; 26_6-6_internal_loop- | AAGCACTCTCCAGGGCGAACTCG | | | | |
| | | symmetric_CUCCUG-AAUCAC | GACACACAAGACAGCCACTGCTG | | | | |
| | | | GGCTGG | | | | |
| 2732 | ABCA4-GA- | −6_6-6_internal_loop-symmetric_GCUGUG- | ATCTTGAATGTGGTTGTTTTGCCG | 0.255 | 1.206 | 0.200 | 1.150 |
| | GC_−6_34 | GCUCCA; −1->0_2-2_bulge-symmetric_GA- | GCACCAGTATAACCCAGGAGGCC | | | | |
| | | CG; 34_6-6_internal_loop- | AAAGCACTCTCCAGGGCGAACTC | | | | |
| | | symmetric_AGUGAA-GUAUAA | GGACAGCTCCACTGTCCACTGCTG | | | | |
| | | | GGCTGG | | | | |
| 2733 | ABCA4-G- | −6_6-6_internal_loop-symmetric_GCUGUG- | ATCTTGAATGTGGTTGTTTTGCCG | 0.376 | 1.310 | 0.162 | 1.080 |
| | G_−6_38 | GUAUGA; −1_1-1_mismatch_G-G; 38_6- | GCTTGGCACACTCCCAGGAGGCC | | | | |
| | | 6_internal_loop-symmetric_AAUGGU- | AAAGCACTCTCCAGGGCGAACTT | | | | |
| | | UUGGCA | GGACAGTATGACTGTCCACTGCTG | | | | |
| | | | GGCTGG | | | | |
| 2734 | ABCA4-G- | −10_6-6_internal_loop-symmetric_ACAGGC- | ATCTTGAATGTGGTTGTTTTGCCG | 0.365 | 1.324 | 0.106 | 1.066 |
| | G_−10_26 | AAGGGG; −1_1-1_mismatch_G-G; 26 6- | GCACCATTCACTCCGCATACGCCA | | | | |
| | | 6_internal_loop-symmetric_CUCCUG- | AAGCACTCTCCAGGGCGAACTTG | | | | |
| | | GCAUAC | GACACACAAAGGGGCCACTGCTG | | | | |
| | | | GGCTGG | | | | |
| 2735 | ABCA4-G- | −8_6-6_internal_loop-symmetric_AGGCUG- | ATCTTGAATGTGGTTGTTTTGCCG | 0.339 | 1.270 | 0.169 | 1.124 |
| | G_−8_34 | AUCUUG; −1_1-1_mismatch_G-G; 34_6- | GCACCAACTGTACCCAGGAGGCC | | | | |
| | | 6_internal_loop-symmetric_AGUGAA- | AAAGCACTCTCCAGGGCGAACTT | | | | |
| | | ACUGUA | GGACACAATCTTGGTCCACTGCTG | | | | |
| | | | GGCTGG | | | | |
| 2736 | ABCA4- | −14_8-7_internal_loop- | ATCTTGAATGTGGTTGTTTTGCCG | 0.431 | 1.388 | 0.382 | 1.294 |
| | fullSHAKER_ | asymmetric_CAGUGGAC-CAGAGAU; - | GCACCATTCACTCCCAGGAGGCGC | | | | |
| | −16 18 | 5_1-0_bulge-asymmetric_U-; −1->0_2- | TCCAACTCTCCAGTGAGAACTCGG | | | | |
| | | 2_bulge-symmetric_GA-CG; 6_1- | ACCACAGCCTCAGAGATCTGGGCT | | | | |
| | | 1_mismatch_G-A; 8_1-1_mismatch_C-U; | GG | | | | |
| | | 18_6-6_internal_loop-symmetric_GCUUUG- | | | | | |
| | | GCUCCA | | | | | |
| 2737 | ABCA4- | −5_7-6_internal_loop- | ATCTTGAATGTGGTTGTTTTGCCG | 0.342 | 1.286 | 0.190 | 1.141 |
| | fullSHAKER_ | asymmetric_GCUGUGU-UUUCUA; −1- | GCTATTTACACTCCCAGGAGGCCA | | | | |
| | −6_38 | >0_2-2_bulge-symmetric_GA-CG; 6_1- | AAGCACTCTCCAGTGAGAACTCG | | | | |
| | | 1_mismatch_G-A; 8_1-1_mismatch_C-U; | GACTTTCTACTGTCCACTGCTGGG | | | | |
| | | 38_6-6_internal_loop-symmetric_AAUGGU- | CTGG | | | | |
| | | UAUUUA | | | | | |
| 2738 | ABCA4- | −12_6-6_internal_loop-symmetric_GGACAG- | ATCTTGAATGTGGTTGTTTTGCCG | 0.256 | 1.210 | 0.154 | 1.101 |
| | fullSHAKER_ | GACCGA; −5_1-0_bulge-asymmetric_U-; −1- | GCACCACGAAGGCCCAGGAGGCC | | | | |
| | −12_34 | >0_2-2_bulge-symmetric_GA-CG; 6_1- | AAAGCACTCTCCAGTGAGAACTC | | | | |
| | | 1_mismatch_G-A; 8_1-1_mismatch_C-U; | GGACCACAGCGACCGAACTGCTG | | | | |
| | | 34_6-6_internal_loop-symmetric_AGUGAA- | GGCTGG | | | | |
| | | CGAAGG | | | | | |
| 2739 | ABCA4- | −10_6-6_internal_loop-symmetric_ACAGGC- | ATCTTGAATGTGGTTGTTTTGCCG | 0.147 | 1.117 | 0.152 | 1.120 |
| | fullSHAKER_ | AAGACG; −5_1-0_bulge-asymmetric_U-; −1- | GCACCATTCACTCCCAGGTTTTTC | | | | |
| | −10_22 | >0_2-2_bulge-symmetric_GA-CG; 6_1- | AAGCACTCTCCAGTGAGAACTCG | | | | |
| | | 1_mismatch_G-A; 8_1-1_mismatch_C-U; | GACCACAAAGACGCCACTGCTGG | | | | |
| | | 22_6-6_internal_loop-symmetric_UGGCCU- | GCTGG | | | | |
| | | UUUUUC | | | | | |
| 2740 | ABCA4- | −16_6-6_internal_loop-symmetric_CAGUGG- | ATCTTGAATGTGGTTGTTTTGCCG | 0.436 | 1.306 | 0.353 | 1.263 |
| | partSHAKER_ | AAUGAU; −1->0_2-2_bulge-symmetric_GA- | GCACCATTCACTCCCAGGAGGCGC | | | | |
| | −16_18 | CG; 6_1-1_mismatch_G-A; 8_1- | ACCAACTCTCCAGTGAGAACTCGG | | | | |
| | | 1_mismatch_C-U; 18_6-6_internal_loop- | ACACACAGCCTGTAATGATCTGGG | | | | |
| | | symmetric_GCUUUG-GCACCA | CTGG | | | | |
| 2741 | ABCA4- | −20_4-4_bulge-symmetric_AGCA-ACAA; | ATCTTGAATGTGGTACAGTTGCCG | 0.387 | 1.337 | 0.174 | 1.114 |
| | partSHAKER_ | −6_6-6_internal_loop-symmetric_GCUGUG- | GCTAGGGCCACTCCCAGGAGGCC | | | | |
| | −6_38_−20- | GUAAUG; −1->0_2-2_bulge-symmetric_GA- | AAAGCACTCTCCAGTGAGAACTC | | | | |
| | 52 | CG; 6_1-1_mismatch_G-A; 8_1- | GGACAGTAATGCTGTCCACACAA | | | | |
| | | 1_mismatch_C-U; 38_6-6_internal_loop- | GGGCTGG | | | | |
| | | symmetric_AAUGGU-UAGGGC; 52_4- | | | | | |
| | | 4_bulge-symmetric_AACA-ACAG | | | | | |
| 2742 | ABCA4- | −10_6-6_internal_loop-symmetric_ACAGGC- | ATCTTGAATGTGGTTGTTTTGCCG | 0.331 | 1.288 | 0.258 | 1.149 |
| | partSHAKER_ | AUGGAC; −1->0_2-2_bulge-symmetric_GA- | GCACACGACTCTCCCAGGAGGCC | | | | |
| | −10_36 | CG; 6_1-1_mismatch_G-A; 8_1- | AAAGCACTCTCCAGTGAGAACTC | | | | |
| | | 1_mismatch_C-U; 36_6-6_internal_loop- | GGACACACAATGGACCCACTGCT | | | | |
| | | symmetric_UGAAUG-ACGACU | GGGCTGG | | | | |
| 2743 | ABCA4- | −8_6-6_internal_loop-symmetric_AGGCUG- | ATCTTGAATGTGGTTGTTTTGCCG | 0.327 | 1.275 | 0.077 | 1.011 |
| | CAPSv0016_ | ACAAGA; −6_1-1_wobble_G-U; −3_1- | GCTATGGCCACTCCCAGGAGGCC | | | | |
| | −8_38 | 1_mismatch_U-U; −1_1-1_mismatch_G-G; | AAAGCACTCTCCAGGGCGAGCTT | | | | |
| | | 3_1-1_wobble_U-G; 38_6-6_internal_loop- | GGTCATAACAAGAGTCCACTGCTG | | | | |
| | | symmetric_AAUGGU-UAUGGC | GGCTGG | | | | |
| 2744 | ABCA4- | −3_7-7_internal_loop- | ATCTTGAATGTGGTTGTTTTGCCG | 0.343 | 1.278 | 0.079 | 1.025 |
| | CAPSv0016_ | symmetric_UGUGUGU-UAGUCUC; −1_1- | GCACCATTCAACGAGTGGAGGCC | | | | |
| | −4_30 | 1_mismatch_G-G; 3_1-1_wobble_U-G; 30_6- | AAAGCACTCTCCAGGGCGAGCTT | | | | |
| | | 6_internal_loop-symmetric_UGGGAG- | GGTAGTCTCGCCTGTCCACTGCTG | | | | |
| | | ACGAGU | GGCTGG | | | | |
| 2745 | ABCA4- | −12_6-6_internal_loop-symmetric_GGACAG- | ATCTTGAATGTGGTTGTTTTGCCG | 0.290 | 1.245 | 0.126 | 1.080 |
| | CAPSv0016_ | AUUUGA; −6_1-1_wobble_G-U; -3_1- | GCACCAGTGTTCCCCAGGAGGCC | | | | |
| | −12_34 | 1_mismatch_U-U; −1_1-1_mismatch_G-G; | AAAGCACTCTCCAGGGCGAGCTT | | | | |
| | | 3_1-1_wobble_U-G; 34_6-6_internal_loop- | GGTCATACAGCATTTGAACTGCTG | | | | |
| | | symmetric_AGUGAA-GUGUUC | GGCTGG | | | | |
| 2746 | ABCA4- | −6_6-6_internal_loop-symmetric_GCUGUG- | ATCTTGAATGTGGTTGTTTTGCCG | 0.290 | 1.200 | 0.171 | 1.110 |
| | CAPSv0016_ | AAGUUA; −3_1-1_mismatch_U-U; −1 1- | GCTTAGCACACTCCCAGGAGGCC | | | | |
| | −6_38 | 1_mismatch_G-G; 3_1-1_wobble_U-G; 38_6- | AAAGCACTCTCCAGGGCGAGCTT | | | | |
| | | 6_internal_loop-symmetric_AAUGGU- | GGTCAAAGTTACTGTCCACTGCTG | | | | |
| | | UUAGCA | GGCTGG | | | | |
| 2747 | ABCA4- | −6_6-6_internal_loop-symmetric_GCUGUG- | ATCTTGAATGTGGTTGTTTTGCCG | 0.307 | 1.260 | 0.094 | 1.044 |
| | CAPSv0015_ | GUUCAA; −3_1-1_mismatch_U-U; −1_1- | GCACACGCTTCTCCCAGGAGGCCA | | | | |
| | −6_36 | 1_mismatch_G-G; 3_1-1_wobble_U-G; 36_6- | AAGCACTCTCCAGGGCGAGCTTG | | | | |
| | | 6_internal_loop-symmetric_UGAAUG- | GTCAGTTCAACTGTCCACTGCTGG | | | | |
| | | ACGCUU | GCTGG | | | | |
| 2748 | ABCA4- | −20_6-6_internal_loop-symmetric_CCAGCA- | ATCTTGAATGTGGTTGTTTTGCCG | 0.277 | 0.989 | 0.102 | 1.006 |
| | CAPSv0015_ | GGGAAA; −3_1-1_mismatch_U-U; −1_1- | GCACCATTCACTCCCAGGAGGCGT | | | | |
| | −20_18 | 1_mismatch_G-G; 3_1-1_wobble_U-G; 18_6- | CTTAACTCTCCAGGGCGAGCTTGG | | | | |
| | | 6_internal_loop-symmetric_GCUUUG- | TCACACAGCCTGTCCACGGGAAA | | | | |
| | | GUCUUA | GCTGG | | | | |
| 2749 | ABCA4- | −12_6-6_internal_loop-symmetric_GGACAG- | ATCTTGAATGTGGTTGTTTTGCCG | 0.272 | 1.232 | 0.136 | 1.093 |
| | CAPSv0015_ | GACGAA; −3_1-1_mismatch_U-U; −1_1- | GCACCATTCACTCCGTCCCAGCCA | | | | |
| | −12_26 | 1_mismatch_G-G; 3_1-1_wobble_U-G; 26_6- | AAGCACTCTCCAGGGCGAGCTTG | | | | |
| | | 6_internal_loop-symmetric_CUCCUG- | GTCACACAGCGACGAAACTGCTG | | | | |
| | | GUCCCA | GGCTGG | | | | |
| 2750 | ABCA4- | −8_6-6_internal_loop-symmetric_AGGCUG- | ATCTTGAATGTGGTTGTTTTGCCG | 0.234 | 1.184 | 0.069 | 1.036 |
| | CAPSv0015_ | AACGGG; −3_1-1_mismatch_U-U; −1 1- | GCACCAAGTGGACCCAGGAGGCC | | | | |
| | −8_34 | 1_mismatch_G-G; 3_1-1_wobble_U-G; 34_6- | AAAGCACTCTCCAGGGCGAGCTT | | | | |
| | | 6_internal_loop-symmetric_AGUGAA- | GGTCACAAACGGGGTCCACTGCT | | | | |
| | | AGUGGA | GGGCTGG | | | | |
| 2751 | ABCA4- | −20_6-6_internal_loop-symmetric_CCAGCA- | ATCTTGAATGTGGTTGTTTTGCCG | 0.491 | 1.307 | 0.169 | 1.125 |
| | SHAKERv0445_ | GAUUAA; −11_1-1_mismatch_G-G; −1- | GCACCATTCACTCCCAGGAGGCAT | | | | |
| | −20_18 | >0_2-2_bulge-symmetric_GA-CG; 7_1- | TAAAACTCTCCAGGACGAACTCG | | | | |
| | | 1_mismatch_C-A; 18_6-6_internal_loop- | GACACACAGGCTGTCCACGATTA | | | | |
| | | symmetric_GCUUUG-AUUAAA | AGCTGG | | | | |
| 2752 | ABCA4- | −6_6-6_internal_loop-symmetric_GCUGUG- | ATCTTGAATGTGGTTGTTTTGCCG | 0.447 | 1.378 | 0.201 | 1.166 |
| | SHAKERv0445_ | AUUCUA; −1->0_2-2_bulge-symmetric_GA- | GCACCAAACCCACCCAGGAGGCC | | | | |
| | −6_34 | CG; 7_1-1_mismatch_C-A; 34_6- | AAAGCACTCTCCAGGACGAACTC | | | | |
| | | 6_internal_loop-symmetric_AGUGAA- | GGACAATTCTACTGTCCACTGCTG | | | | |
| | | AACCCA | GGCTGG | | | | |
| 2753 | ABCA4- | −14_6-6_internal_loop-symmetric_GUGGAC- | ATCTTGAATGTGGTTGTTTTGCCG | 0.406 | 1.363 | 0.185 | 1.139 |
| | SHAKERv0445_ | ACAUUG; −11_1-1_mismatch_G-G; −1->0_ | GCACCATTCACTCCCAGGTGAGAT | | | | |
| | −14_22 | 2-2_bulge-symmetric_GA-CG; 7_1- | AAGCACTCTCCAGGACGAACTCG | | | | |
| | | 1_mismatch_C-A; 22_6-6_internal_loop- | GACACACAGGCTACATTGTGCTGG | | | | |
| | | symmetric_UGGCCU-UGAGAU | GCTGG | | | | |
| 2754 | ABCA4- | −6_6-6_internal_loop-symmetric_GCUGUG- | ATCTTGAATGTGGTTGTTTTGCCG | 0.366 | 1.300 | 0.349 | 1.284 |
| | SHAKERv0445_ | ACGUUG; −1->0_2-2_bulge-symmetric_GA- | GCTTTTACCACTCCCAGGAGGCCA | | | | |
| | −6_38 | CG; 7_1-1_mismatch_C-A; 38_6- | AAGCACTCTCCAGGACGAACTCG | | | | |
| | | 6_internal_loop-symmetric_AAUGGU- | GACAACGTTGCTGTCCACTGCTGG | | | | |
| | | UUUUAC | GCTGG | | | | |
| 2755 | ABCA4- | −10_6-6_internal_loop-symmetric_ACAGGC- | ATCTTGAATGTGGTTGTTTTGCCG | 0.395 | 1.349 | 0.210 | 1.168 |
| | SHAKERv0445_ | UAAUUA; −1->0_2-2_bulge-symmetric_GA- | GCACCATTCACTCCCATACTTACA | | | | |
| | −10_24 | CG; 7_1-1_mismatch_C-A; 24_6- | AAGCACTCTCCAGGACGAACTCG | | | | |
| | | 6_internal_loop-symmetric_GCCUCC- | GACACACATAATTACCACTGCTGG | | | | |
| | | UACUUA | GCTGG | | | | |
| 2756 | ABCA4- | −20_6-6_internal_loop-symmetric_CCAGCA- | ATCTTGAATGTGGTTGTTTTGCCG | 0.461 | 1.015 | 0.177 | 1.116 |
| | AJUBAv0445_ | ACGACC; −10_1-1_mismatch_C-A; −1_1- | GCACCATTCACTCCCAGGAGGCGT | | | | |
| | −20_18 | 1_mismatch_G-G; 3_1-1_mismatch_U-U; | CCTAACTCTCCAGGGCGATCTTGG | | | | |
| | | 18_6-6_internal_loop-symmetric_GCUUUG- | ACACACAACCTGTCCACACGACC | | | | |
| | | GUCCUA | GCTGG | | | | |
| 2757 | ABCA4- | −6_6-6_internal_loop-symmetric_GCUGUG- | ATCTTGAATGTGGTTGTTTTGCCG | 0.379 | 1.322 | 0.104 | 1.063 |
| | AJUBAv0445_ | GUUACA; −1_1-1_mismatch_G-G; 3_1- | GCACGTTACCCTCCCAGGAGGCCA | | | | |
| | −6_36 | 1_mismatch_U-U; 36_6-6_internal_loop- | AAGCACTCTCCAGGGCGATCTTGG | | | | |
| | | symmetric_UGAAUG-GUUACC | ACAGTTACACTGTCCACTGCTGGG | | | | |
| | | | CTGG | | | | |
| 2758 | ABCA4- | −14_6-6_internal_loop-symmetric_GUGGAC- | ATCTTGAATGTGGTTGTTTTGCCG | 0.316 | 1.260 | 0.223 | 1.152 |
| | AJUBAv0445_ | CAUAGA; −10_1-1_mismatch_C-A; −1_1- | GCACCATTCACTCCCAGGAGATTC | | | | |
| | −14_20 | 1_mismatch_G-G; 3_1-1_mismatch_U-U; | TTGCACTCTCCAGGGCGATCTTGG | | | | |
| | | 20_6-6_internal_loop-symmetric_UUUGGC- | ACACACAACCTCATAGATGCTGG | | | | |
| | | AUUCUU | GCTGG | | | | |
| 2759 | ABCA4- | −8_6-6_internal_loop-symmetric_AGGCUG- | ATCTTGAATGTGGTTGTTTTGCCG | 0.371 | 1.315 | 0.247 | 1.200 |
| | AJUBAv0445_ | ACUCGG; −1_1-1_mismatch_G-G; 3_1- | GCTAAAACCACTCCCAGGAGGCC | | | | |
| | −8_38 | 1_mismatch_U-U; 38_6-6_internal_loop- | AAAGCACTCTCCAGGGCGATCTTG | | | | |
| | | symmetric_AAUGGU-UAAAAC | GACACAACTCGGGTCCACTGCTGG | | | | |
| | | | GCTGG | | | | |
| 2760 | ABCA4- | −14_6-6_internal_loop-symmetric_GUGGAC- | ATCTTGAATGTGGTTGTTTTGCCG | 0.252 | 1.207 | 0.096 | 1.051 |
| | AJUBAv0445_ | UUAAAA; −10_1-1_mismatch_C-A; −1_1- | GCACCATTCACTCCCACTTCTACA | | | | |
| | −14_24 | 1_mismatch_G-G; 3_1-1_mismatch_U-U; | AAGCACTCTCCAGGGCGATCTTGG | | | | |
| | | 24_6-6_internal_loop-symmetric_GCCUCC- | ACACACAACCTTTAAAATGCTGGG | | | | |
| | | CUUCUA | CTGG | | | | |
| 2761 | ABCA4- | −6_6-6_internal_loop-symmetric_GCUGUG- | ATCTTGAATGTGGTTGTTTTGCCG | 0.308 | 1.248 | 0.215 | 1.164 |
| | AJUBAv0445_ | GCGUUG; −1_1-1_mismatch_G-G; 3_1- | GCACCAGCCCAACCCAGGAGGCC | | | | |
| | −6_34 | 1_mismatch_U-U; 34_6-6_internal_loop- | AAAGCACTCTCCAGGGCGATCTTG | | | | |
| | | symmetric_AGUGAA-GCCCAA | GACAGCGTTGCTGTCCACTGCTGG | | | | |
| | | | GCTGG | | | | |

### Example 26

Exemplary ABCA4 Guide RNA that Facilitates Editing of ABCA4 Having a 5′ G

One of the most common missense mutations implicated in Stargardt disease is a G>A mutation (e.g., the c.5882 G>A mutation resulting in the G1961E amino acid mutaiton), which can be corrected by ADAR. However, ADAR enzymes may be generally disinclined to deaminate adenosines with an upstream 5′G, as is the case with targeting the c.5882G>A mutation.

An exemplary engineered guide RNA targeting ABCA4 0.92.62 (−15,+33) (SEQ ID NO: 101) (see also EXAMPLE 9) having a sequence of “CAGG” immediately upstream of the A/C mismatch was evaluated for the ability to facilitate ADAR-mediated RNA editing of an adenosine with a 5′G in ABCA4. Briefly, 750 ng of plasmid encoding an engineered guide RNA was delivered to HEK293 cells having an ABCA4 cDNA minigene. Data was collected 48 hours post-transfection. As shown in FIG. 118, the ABCA4 0.92.62 (−15,+33) engineered guide RNA of SEQ ID NO: 101 facilitates editing of the target adenosine at an efficiency of 55% in the presence of the 5′G.

Thus, this example demonstrates that an engineered guide RNA having a CAGG sequence motif prior to the A/C mismatch is capable of facilitating ADAR-mediated RNA editing of an ABCA4 mRNA having a 5′G directly upstream of the target adenosine.

### Example 27

### Design SERPINA1-Targeting Engineered Guide RNAs

This example describes design of SERPINA1 engineered guide RNAs having barbell macro-footprints. Engineered guide RNAs were designed using the process shown in FIG. 78 (see also EXAMPLE 23): 1. Positioning of the macro-footprint; 2. Fine-tuning of the left-barbell and right-barbell coordinates; and 3. Shortening of the guide length.

As shown in FIG. 119, engineered guide RNAs were designed by tiling the position of the barbell macro-footprint along the guide-target RNA scaffold to identify those engineered guide RNAs that facilitate the highest level of SERPINA1 editing. As shown in FIG. 119, the engineered guide RNA having a 6/6 right internal symmetric loop (“right barbell”) at position +34, a left barbell at position −12, and a centrally positioned mismatch (06566 0.100.50 (−12, +34)) facilitated an ADAR-mediated RNA editing efficiency of 24%. This macro-footprint positioning was carried forward for further engineering.

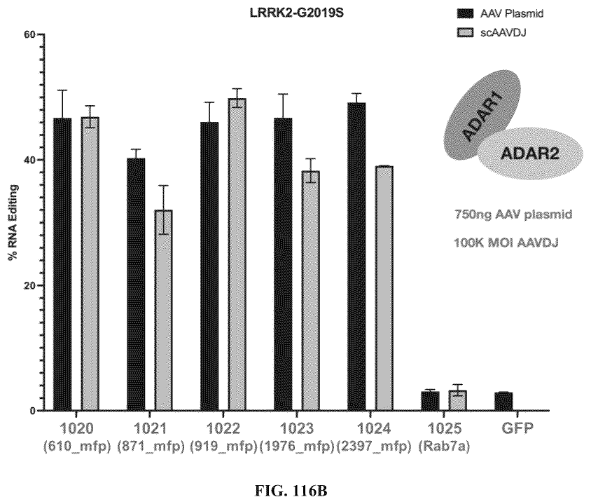

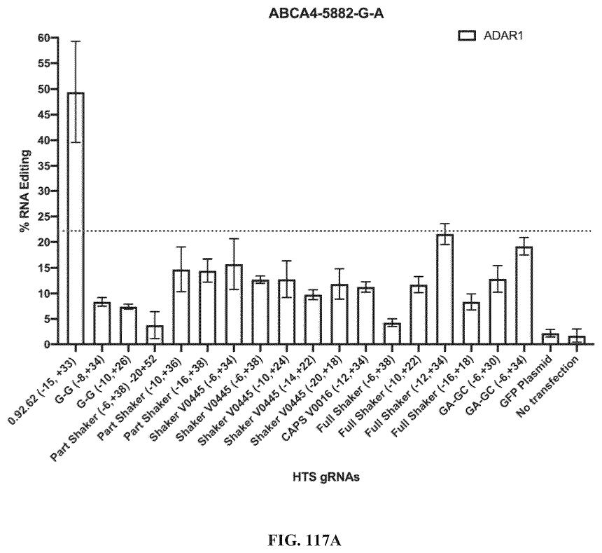

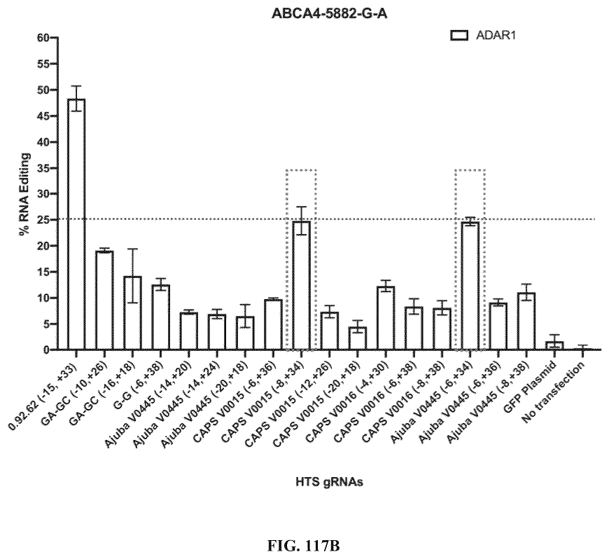

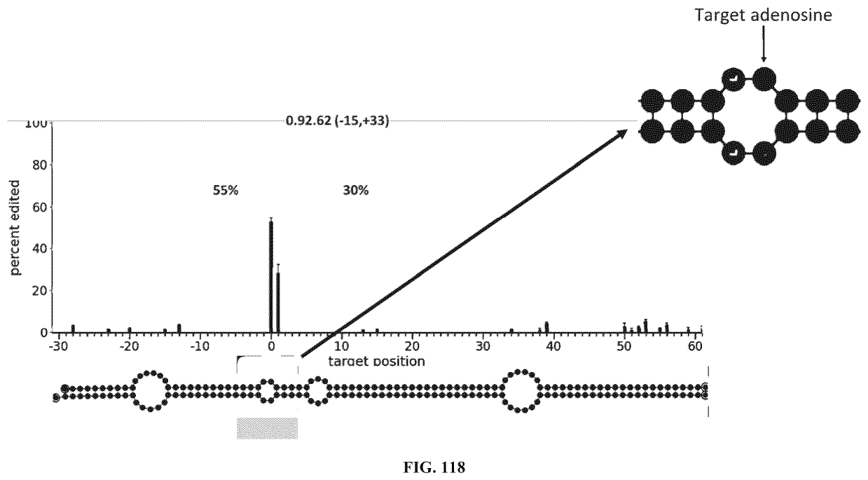

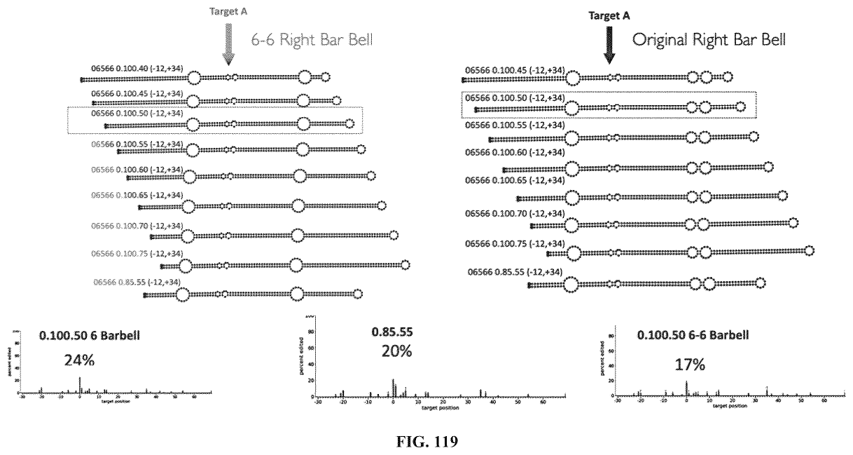

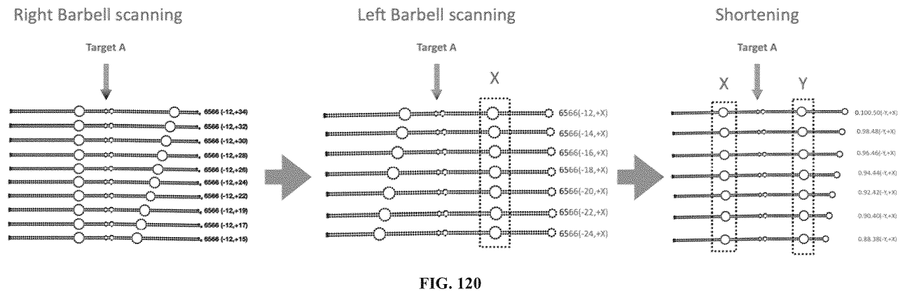

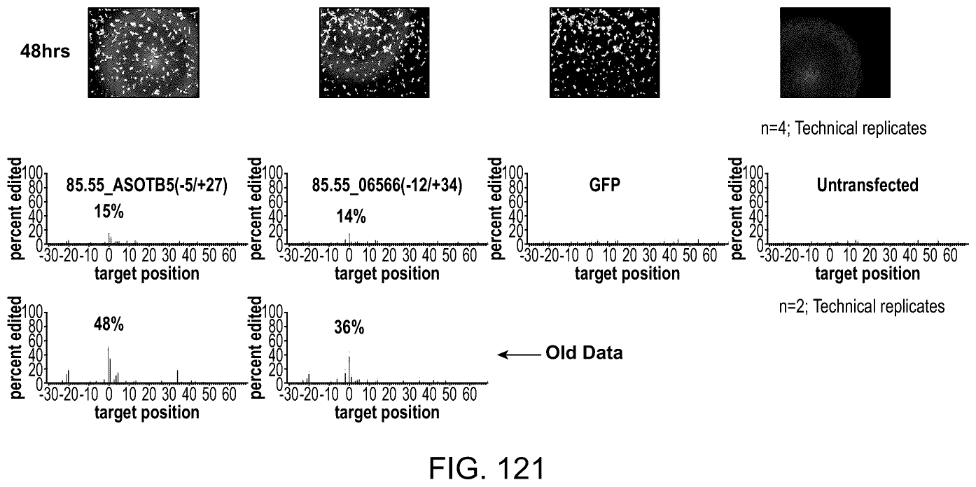

FIG. 120 shows design of the right and left barbell coordinates, as well as engineering of guide length for the 06566 engineered guide RNA.

TABLE 15 below recites the sequences of the engineered guide RNAs depicted in FIGS. 119 and 120. Polynucleotide sequences encoding the engineered guide RNA sequences in TABLE 15 are also encompassed herein.

TABLE 15
Engineered SERPINA1 Guide RNA Sequences

| SEQ | | | |
|---|---|---|---|
| ID | | | |
| NO | FIG. | Guide Name | Sequence |
| 3133 | 119 | SERP_006566_100.50_ | CAUGGGUAUGGCCUCUAAAAACAUGGCCCCUCGUCGUUC |
| | 120 | (−12_+15) | AGUCCUUACUCGUCGAUGGUCAUAAGUUCCUUAUGCACG |
| | | | GCCUUGGAGAGCUUCAGGGGUG |
| 3134 | 119 | SERP_006566_100.50_ | CAUGGGUAUGGCCUCUAAAAACAUGGCCGGUCGUGCUUC |
| | 120 | (−12_+17) | AGUCCUUACUCGUCGAUGGUCAUAAGUUCCUUAUGCACG |
| | | | GCCUUGGAGAGCUUCAGGGGUG |
| 3135 | 119 | SERP_006566_100.50_ | CAUGGGUAUGGCCUCUAAAAACAUGGAGGGUCCAGCUUC |
| | 120 | (−12_+19) | AGUCCUUACUCGUCGAUGGUCAUAAGUUCCUUAUGCACG |
| | | | GCCUUGGAGAGCUUCAGGGGUG |
| 3136 | 119 | SERP_006566_100.50_ | CAUGGGUAUGGCCUCUAAAAACAUCCAGGCAGCAGCUUC |
| | 120 | (−12_+22) | AGUCCUUACUCGUCGAUGGUCAUAAGUUCCUUAUGCACG |
| | | | GCCUUGGAGAGCUUCAGGGGUG |
| 3137 | 119 | SERP_006566_100.50_ | CAUGGGUAUGGCCUCUAAAAAGUCCCACCCAGCAGCUUC |
| | 120 | (−12_+24) | AGUCCUUACUCGUCGAUGGUCAUAAGUUCCUUAUGCACG |
| | | | GCCUUGGAGAGCUUCAGGGGUG |
| 3138 | 119 | SERP_006566_100.50_ | CAUGGGUAUGGCCUCUAAAUUGUCCGCCCCAGCAGCUUC |
| | 120 | (−12_+26) | AGUCCUUACUCGUCGAUGGUCAUAAGUUCCUUAUGCACG |
| | | | GCCUUGGAGAGCUUCAGGGGUG |
| 3139 | 119 | SERP_006566_100.50_ | CAUGGGUAUGGCCUCUAUUCCGUUGGCCCCAGCAGCUUC |
| | 120 | (−12_+28) | AGUCCUUACUCGUCGAUGGUCAUAAGUUCCUUAUGCACG |
| | | | GCCUUGGAGAGCUUCAGGGGUG |
| 3140 | 119 | SERP_006566_100.50_ | CAUGGGUAUGGCCUCGGUUCCCAUGGCCCCAGCAGCUUC |
| | 120 | (−12_+30) | AGUCCUUACUCGUCGAUGGUCAUAAGUUCCUUAUGCACG |
| | | | GCCUUGGAGAGCUUCAGGGGUG |
| 3141 | 119 | SERP_006566_100.50_ | CAUGGGUAUGGCCAGGGUUAACAUGGCCCCAGCAGCUUC |
| | 120 | (−12_+32) | AGUCCUUACUCGUCGAUGGUCAUAAGUUCCUUAUGCACG |
| | | | GCCUUGGAGAGCUUCAGGGGUG |
| 3142 | 119 | SERP_006566_100.50_ | CAUGGGUAUGGGGAGGUAAAACAUGGCCCCAGCAGCUUC |
| | 120 | (−12_+34) | AGUCCUUACUCGUCGAUGGUCAUAAGUUCCUUAUGCACG |
| | | | GCCUUGGAGAGCUUCAGGGGUG |
| 3143 | 119 | SERP_ASOTB5_85.55_ | UAGACAUGGGUAUGGCCUCUAAUUUGUAGGCCCCAGCAG |
| | 120 | (−5_+27) | CUUCAGUCCCUUACUCGUCGUACCAGAGCACAGCCAGUCG |
| | | | UAUGCACGGC |
| 3144 | 119 | SERP_006566_85.55_ | UAGACAUGGGUAUGGGGCAGUAUUCCCAUGGCCCCAGCA |
| | 120 | (−12_+34) | GCUUCAGUCCUUACUCGUCGAUGGUCAUAAGUUCCUUAU |
| | | | GCACGGC |

### Example 28

### Selection of SERPINA1 Engineered Guide RNAs for AAV Packaging

The following guides recited in TABLE 16 targeting SERPINA1 having barbell macro-footprints were selected for packaging into AAV vectors. While the engineered guide RNA sequences in TABLE 16 are provided as DNA sequences with a T substituted for each U, the corresponding RNA sequences are also encompassed herein.

TABLE 16
Exemplary guide RNAs targeting SERPINA1

| SEQ | | |
|---|---|---|
| ID | | |
| NO | Guide Name | Sequence |
| 2762 | 85.55_SERP_−5_27 | TAGACATGGGTATGGCCTCTAATTTGTAGGCCCCA |
| | | GCAGCTTCAGTCCCTTACTCGTCGTACCAGAGCAC |
| | | AGCCTTATGCACGGC |
| 2763 | 85.55_SERP-ASOTB5_−5_27 | TAGACATGGGTATGGCCTCTAATTTGTAGGCCCCA |
| | | GCAGCTTCAGTCCCTTACTCGTCGTACCAGAGCAC |
| | | AGCCAGTCGTATGCACGGC |
| 2764 | 85,55_SERP_6566_−12_34 | TAGACATGGGTATGGGGCAGTATTCCCATGGCCCC |
| | | AGCAGCTTCAGTCCTTACTCGTCGATGGTCATAAG |
| | | TTCCTTATGCACGGC |
| 2765 | 85.55_SERP-010980_−8_38 | TAGACATGGGTCACAAGTCTAAAAACAATACTAC |
| | | CCAGCAGCTTCAGTCCACCTCGTCGATGACAACGA |
| | | CAGCCTTATGCACGGC |
| 2766 | 85.55_SERP-011202_−4_38 | TAGACATGGGTCCATAGTCTAATCCATGGCTACCC |
| | | CAGCAGCTTGTCCCTTTCTCGTCACCAGACAGCAC |
| | | AGCCTTATGCACGGC |
| 2767 | 85.55_SERP-010900_−6_36 | TAGACATGGGTATATAAGGTAAAAACATGTACGC |
| | | CCCAGCCTTCAGTCCCTTCTCGTCGAACTATTGCA |
| | | CAGCCTTATGCACGGC |
| 2768 | 85.55_SERP-AC-AA_−19_27 | TAGACATGGGTATGGCCTCTAATTTGTAGGCCCCA |
| | | GCAGCTTCAGTCCCTTACTCGTCGATGGTCAGCAC |
| | | AGCGAATACCACGGC |
| 3083 | SERP-AC-AA_95-50_−10_26 | ATGGGTATGGCCTCTAAACCCCGAGCCCCAGCAG |
| | | CTTCAGTCCCTTACTCGTCGATGGTGTAAAAAGCC |
| | | TTATGCACGGCCTGGAGGGGAGAGAA |
| 3084 | SERP-AC-AA_95-50_−10_25 | ATGGGTATGGCCTCTAAAATGTCCCCCCCAGCAGC |
| | | TTCAGTCCCTTACTCGTCGATGGTGCTATAAGCCT |
| | | TATGCACGGCCTGGAGGGGAGAGAA |
| 3085 | SERP-AC-AA_95-50_−8_28 | ATGGGTATGGCCTCTACCCTGTTGGCCCCAGCAGC |
| | | TTCAGTCCCTTACTCGTCGATGAAGACGACAGCCT |
| | | TATGCACGGCCTGGAGGGGAGAGAA |
| 3086 | SERP-100.50-position_−20 | CATGGCCCCAGCAGCTTCAGTCCCTTTCTTGTCGA |
| | adenosine scan control | TGGTCAGCACAGCCCTATGCACGGCCTGGAGGGG |
| | | AGAGAAGCAGAGACACGTTGTAAGGCTGATC |

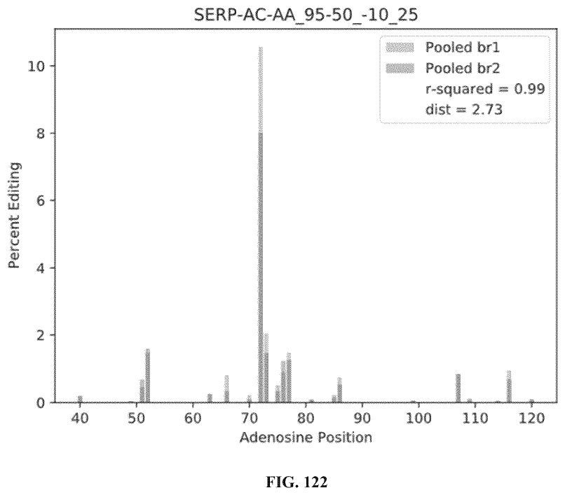

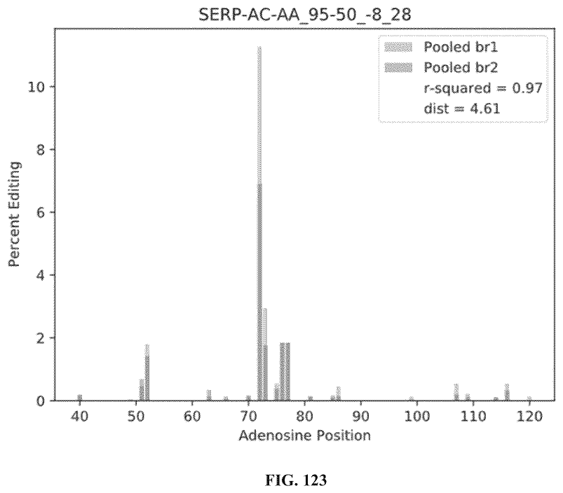

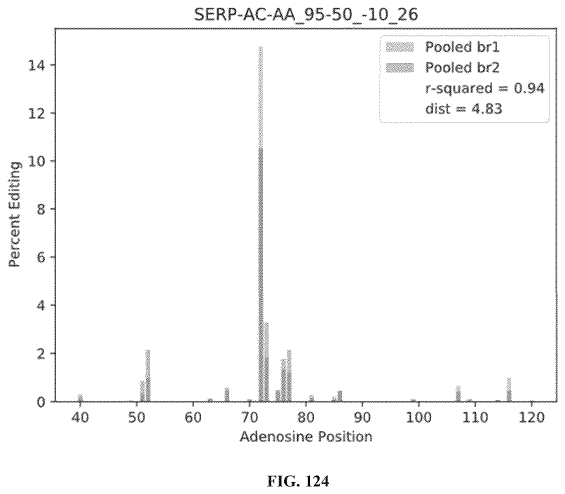

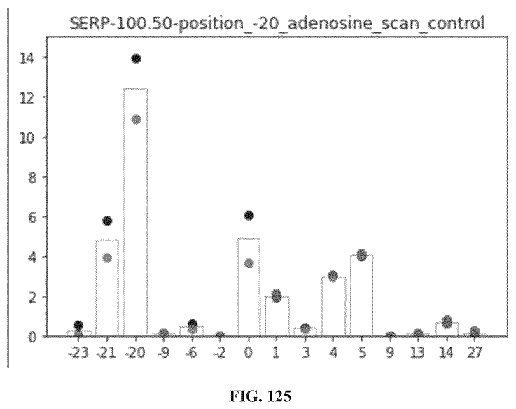

FIG. 122-FIG. 125 depict RNA editing efficiencies for guides SERP-AC-AA_95-50-10_25 (FIG. 122—SEQ ID NO: 3084); SERP-AC-AA_95-50_-8_28 (FIG. 123—SEQ ID NO: 3085); SERP-AC-AA_95-50_-10_26 (FIG. 124—SEQ ID NO: 3083) and SERP-100.50-position_-20 adenosine scan control (FIG. 125—SEQ ID NO: 3086) via ADAR.

### Example 29

### In Cell Screening of Engineered Guide RNAs Against ABCA4

A lead engineered guide RNA design of SEQ ID NO: 2772 was packaged into an AAV vector and delivered to HEK293 cells expressing the ABCA4 G1961E mutation and endogenous ADAR1, as well as ARPE-19 cells expressing the ABCA4 G1961E mutation and endogenous ADAR1 and ADAR2. 10,000 cells were plated per well in a 96-well plate and infected with 100,000 vg/dg of the scAAV2-ABCA4-Thy1.1 construct. After 48 hours, cells were harvested for analysis:
Flow cytometry to detect Thy1.1 fluorescence to assess transduction efficiency
RNA extraction→cDNA synthesis→PCR to amplify the ABCA4 target site→Sanger sequence to diagnose % editing

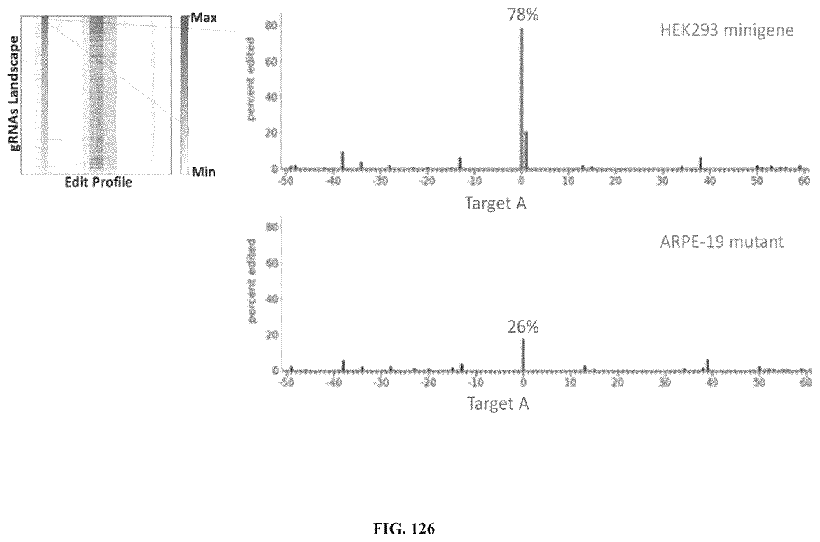

FIG. 126 shows the editing profile for the engineered guide RNA of SEQ ID NO: 2772 via endogenous ADAR1 for the ABCA4 target RNA in the human cells. The optimized gRNA obtained 78% editing (HEK minigene cells) and 26% editing (ARPE-19 cells) with no detectable non-synonymous bystander editing.

The data highlight the ability of the RNAfix platform the achieve robust and specific editing of a challenging 5′G target with endogenous ADAR when delivered by AAV.

### Example 30

### Editing of LRRK2 by Engineered Guide RNA Using a Broken GFP Reporter

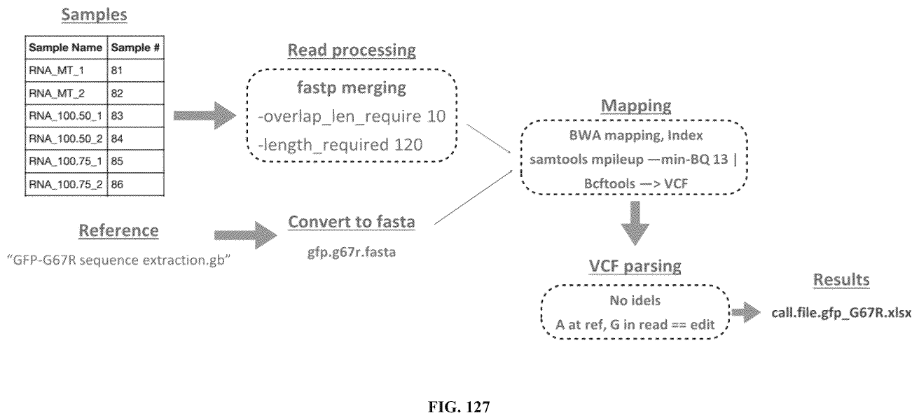

100 nucleotide engineered guide RNAs with an A/C mismatch at positions 25, 50, and 75 with LRRK2 guide mimicry or barbells at different positions were tested in K562 cells expressing a GFP-G67R reporter. FIG. 127 depicts a workflow for screening exemplary guide RNAs targeting LRRK2 in a broken GFP reporter system. The cells were selected by puromycin to enrich for plasmid and guide integration. The WT ADAR results were from cells captured following 14 days of puro selection and for the ADAR2 overexpression (with a weak constitutive promoter, PGK) 21 days of selection. The editing was assessed by NGS sequencing on the iSeq instrument.

TABLE 17 below recites the engineered guide RNA sequences utilized in this example. While the engineered guide RNA sequences in TABLE 17 are provided as DNA sequences with a T substituted for each U, the corresponding RNA sequences are also encompassed herein.

TABLE 17
Engineered LRRK2 Guide RNA Sequences utilized in Broken GFP Reporter
System

| SEQ | | | |
|---|---|---|---|
| ID | | | |
| NO | FIG. | Guide Name | Sequence |
| 3087 | 128 | 100.75_AC_CO1 | GCCTTCGGGCATGGCGGACTTGAAGAAGTCGTGCTGCTTCAT |
| | | | GTGGTCGGGGTAGCGGCTGAAGCACTGCACTCCGTAGGTCA |
| | | | GGGTCGTCACGAGGGTC |
| 3088 | 128 | 100.50_AC_CO1 | AAGTCGTGCTGCTTCATGTGGTCGGGGTAGCGGCTGAAGCAC |
| | | | TGCACTCCGTAGGTCAGGGTCGTCACGAGGGTCGGCCAGGG |
| | | | CACGGGCAGCTTGCCGG |
| 3089 | 128 | 100.25_AC_CO1 | GGTAGCGGCTGAAGCACTGCACTCCGTAGGTCAGGGTCGTC |
| | | | ACGAGGGTCGGCCAGGGCACGGGCAGCTTGCCGGTGGTGCA |
| | | | GATGAACTTCAGGGTCAG |
| 3090 | 128 | 0.100.75_(LRRK2_1976_ | GCCTTCGGGCATGGCGGACTTGAAGAAGTCGTGCTGCTTCAT |
| | | mimicry) | GTGGTCGGGGTAGCGCCTGAAGCACTGGACAGCCCTCGTCTG |
| | | | GGTCGTCACGAGGGTC |
| 3091 | 128 | 0.100.75_(LRRK2_919_ | GCCTTCGGGCATGGCGGACTTGAAGAAGTCGTGCTGCTTCAT |
| | | mimicry) | GTGGTCGGGGTAGCGGCTGATCAACTCCACAGCCAAGGTCA |
| | | | GGGTCGTCACGAGGGTC |
| 3092 | 128 | 0.100.75_(LRRK2_871_ | GCCTTCGGGCATGGCGGACTTGAAGAAGTCGTGCTGCTTCAT |
| | | mimicry) | GTGGTCGGGGTAGCGGCTGTAGCACTGCCCTCCCATCCACAG |
| | | | GGTCGTCACGAGGGTC |
| 3093 | 128 | 0.100.75_(LRRK2_860_ | GCCTTCGGGCATGGCGGACTTGAAGAAGTCGTGCTGCTTCAT |
| | | mimicry) | GTGGTCGGGGTAGCGGCTGAAGGACTCGTCTCCCAAGGTCTG |
| | | | GGTCGTCACGAGGGTC |
| 3094 | 128 | 0.100.50_(LRRK2_1976_ | AAGTCGTGCTGCTTCATGTGGTCGGGGTAGCGCCTGAAGCAC |
| | | mimicry) | TGGACAGCCCTCGTCTGGGTCGTCACGAGGGTCGGCCAGGG |
| | | | CACGGGCAGCTTGCCGG |
| 3095 | 128 | 0.100.50_(LRRK2_919_ | AAGTCGTGCTGCTTCATGTGGTCGGGGTAGCGGCTGATCAAC |
| | | mimicry) | TCCACAGCCAAGGTCAGGGTCGTCACGAGGGTCGGCCAGGG |
| | | | CACGGGCAGCTTGCCGG |
| 3096 | 128 | 0.100.50_(LRRK2_871_ | AAGTCGTGCTGCTTCATGTGGTCGGGGTAGCGGCTGTAGCAC |
| | | mimicry) | TGCCCTCCCATCCACAGGGTCGTCACGAGGGTCGGCCAGGGC |
| | | | ACGGGCAGCTTGCCGG |
| 3097 | 128 | 0.100.50_(LRRK2_860_ | AAGTCGTGCTGCTTCATGTGGTCGGGGTAGCGGCTGAAGGAC |
| | | mimicry) | TCGTCTCCCAAGGTCTGGGTCGTCACGAGGGTCGGCCAGGGC |
| | | | ACGGGCAGCTTGCCGG |
| 3098 | 128 | −30, +6_0.100.25 | GGTAGCGGCTGAACGTGACCACTCCGTAGGTCAGGGTCGTC |
| | | | ACGAGGGTCGGCCTCCCGTCGGGCAGCTTGCCGGTGGTGCA |
| | | | GATGAACTTCAGGGTCAG |
| 3099 | 128 | −30, +5_0.100.25 | GGTAGCGGCTGAAGGTGACGACTCCGTAGGTCAGGGTCGTC |
| | | | ACGAGGGTCGGCCTCCCGTCGGGCAGCTTGCCGGTGGTGCA |
| | | | GATGAACTTCAGGGTCAG |
| 3100 | 128 | −6, +30_0.100.75 | GCCTTCGGGCATGGCGGACTTGAAGAAGTCGTGCTGCTTGTA |
| | | | CACGTCGGGGTAGCGGCTGAAGCACTGCACTCCGTAGGAGT |
| | | | CCCTCGTCACGAGGGTC |
| 3101 | 128 | −6, +30_0.100.50 | AAGTCGTGCTGCTTGTACACGTCGGGGTAGCGGCTGAAGCAC |
| | | | TGCACTCCGTAGGAGTCCCTCGTCACGAGGGTCGGCCAGGGC |
| | | | ACGGGCAGCTTGCCGG |
| 3102 | 128 | −5, +30_0.100.75 | GCCTTCGGGCATGGCGGACTTGAAGAAGTCGTGCTGCTTGTA |
| | | | CACGTCGGGGTAGCGGCTGAAGCACTGCACTCCGTAGTAATC |
| | | | CGTCGTCACGAGGGTC |

FIG. 128 depicts the editing efficiency of the exemplary guides targeting LRRK2 in the broken GFP reporter system via exogenous or endogenous ADAR.

### Example 31

### In Vivo Efficacy of an Exemplary Engineered Guide RNA Targeting SERPINA1

### In Vivo Experimental Design.

All animal procedures were performed in accordance with protocol.

The plasmid containing the 85,55_BB(−5/+27)_ASOTB5 guide sequence (SEQ ID NO: 2763-TAGACATGGGTATGGCCTCTAATTTGTAGGCCCCAGCAGCTTCAGTCCCTTACTCGT CGTACCAGAGCACAGCCAGTCGTATGCACGGC) and mCherry as transduction marker was packaged in ssAAV8 and was injected intravenously into the lateral tail vein of male mice, 8 weeks of age, at a dose of 3.74 E12 vector genomes per mouse. Mice were monitored twice per week for the duration of the experiment (4 weeks).

### Tissue Collection and Processing & Ex Vivo Readouts

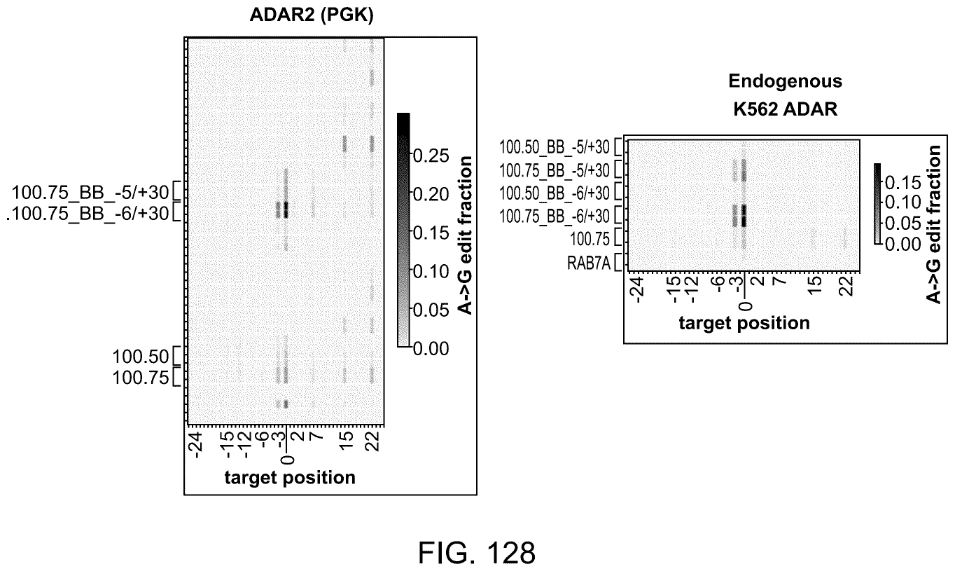

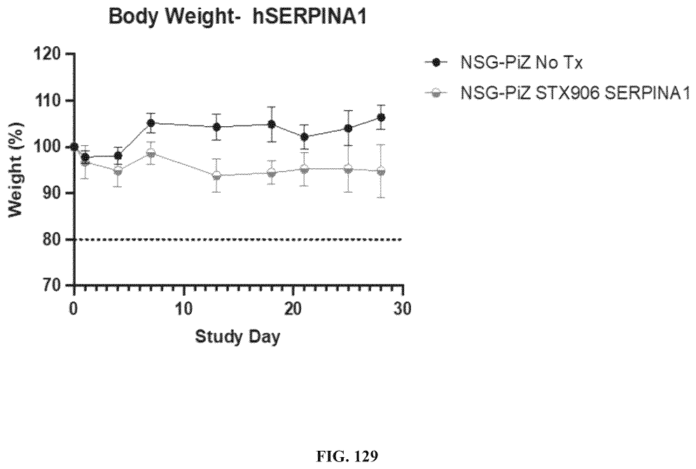

On Day 28 post-injection, all study animals were euthanized. One piece of the right lobe of the liver (approximately 0.5−1 cm³ or 50-100 mg) was collected into cryovials and snap-frozen in LN2 to be processed for RNA for guide expression and detection of editing. The remainder of the whole liver was dissociated using a kit manufacturer's protocol. Dissociated liver cells were used to evaluate transduction based on mCherry expression by flow cytometry staining. FIG. 129 shows the change in body weight for each subject over the course of the 28 day study.

### Flow Cytometry for Evaluating Transduction

Dissociated liver cells were stained with Fixable Yellow viability dye, mouse CD45 and Ter 19, ASGR1, and EpCam and evaluated for transduction by mCherry expression. FIG. 130 illustrates transduction of the engineered guide RNA payload in the liver, as measured by expression of the mCherry reporter. Data collected on the Attune N×T flow cytometer and analyzed using FlowJo flow analysis software.

### RNA Extraction

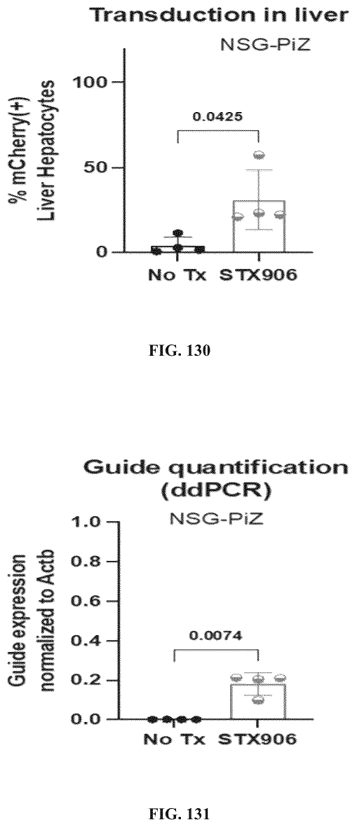

RNA was extracted from cells resuspended in RNA Lysis buffer and RNA was eluted in 20 μL of elution solution. RNA was quantified and cDNA synthesized from 100 ng RNA. FIG. 131 depicts normalized quantitation of the engineered guide RNA.

### Sanger Sequencing

PCR amplification for hSERPINA1 was performed on 1:10 diluted cDNA for 35 cycles using the below primers, and post-amplification analysis performed with 1 uL of PCR product using 2% E-Gel. SERPINA1 Fwd Primer: TGTCCATTACTGGAACCTATGATCTG—SEQ ID NO: 3103 (Tm=56C) SERPINA1 Rvs Primer: GATTCACCACTTTTCCCATGAAGAG—SEQ ID NO: 3104 (Tm=56C)

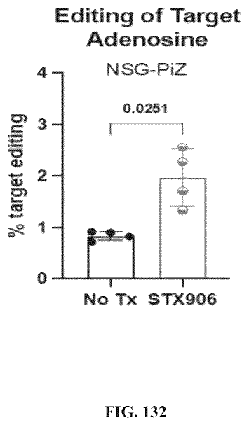

FIG. 132 depicts quantitation of the amount of target adenosine editing of the SERPINA1 RNA through administration of the AAV vector encoding the engineered guide RNA targeting SERPINA1, as compared to the level of editing of the control AAV vector. As depicted in FIG. 132, the engineered guide RNA facilitated an increased in ADAR-mediated editing of the target adenosine of the SERPINA1 RNA, relative to the amount of editing achieved through administration of the control vector. Thus, this example demonstrates that administration of an AAV vector encoding an engineered guide RNA targeting SERPINA1 to a subject produces editing of the target SERPINA1 mRNA in vivo.

### Example 32

### Editing Efficiency of Exemplary Circularized Engineered Guide RNAs Targeting LRRK2

HEK cells expressing endogenous ADARs and the LRRK2 minigene were utilized for these experiments. Specifically, 20,000 cells were transfected with 750 ng of plasmid and 3 μL Trans-IT 293 by reverse transfections. Cells were harvested 48 h post transfections. All linear gRNA were in expressed in a plasmid encoded U1 SmOpt format. All circular gRNA were created by flanking the antisense sequence with ribozymes, expressed from a U6 promoter in a plasmid encoded format.

TABLE 18 below contains the sequences of the engineered guide RNAs used in this example. Underlined nucleotides in the circular gRNA denote the ribozyme, ligation stem and golden gate scar.

TABLE 18
Engineered Linear and Circularized LRRK2 Guide RNA Sequences utilized
in Example 32

| SEQ | | | |
|---|---|---|---|
| ID | | | |
| NO | FIG. | Guide Name | Sequence |
| 2993 | 133 | Linear 871_98.55 | CUGGUGUGCCCUCUGAUCCAAGCAUCCCCAUUCUACAG |
| | | | UAGGACUGAGCACUGCCGAGCUGGGCAAUCUUGAGUCG |
| | | | GAUGGCAGCAUUGGGAUACAGU |
| 3105 | 133 | Circular 871_98.55 | GGCCAUCAGUCGCCGGUCCCAAGCCCGGAUAAAAUGGG |
| | | | AGGGGGCGGGAAACCGCCUAACCAUGCCGACUGAUGGC |
| | | | AGAUAAUCUGGUGUGCCCUCUGAUCCAAGCAUCCCCAU |
| | | | UCUACAGUAGGACUGAGCACUGCCGAGCUGGGCAAUCU |
| | | | UGAGUCGGAUGGCAGCAUUGGGAUACAGUAUAUACUGC |
| | | | CAUCAGUCGGCGUGGACUGUAGAACACUGCCAAUGCCG |
| | | | GUCCCAAGCCCGGAUAAAAGUGGAGGGUACAGUCCACG |
| | | | CUUUUUUU |
| 3037 | 133 | Linear 919_96.50 | GUGCCCUCUGAUGUUUUUAUCCGGCGAGUACAGCAGUU |
| | | | CGGAGGAAUCCCGAGGUCAGCAAUCCUAAGUAUGAUGG |
| | | | CAGCAUUGGGAUACAGUGUG |
| 3106 | 133 | Circular 919_96.50 | GGCCAUCAGUCGCCGGUCCCAAGCCCGGAUAAAAUGGG |
| | | | AGGGGGGGGGAAACCGCCUAACCAUGCCGACUGAUGGC |
| | | | AGAUAAUGUGCCCUCUGAUGUUUUUAUCCGGCGAGUAC |
| | | | AGCAGUUCGGAGGAAUCCCGAGGUCAGCAAUCCUAAGU |
| | | | AUGAUGGCAGCAUUGGGAUACAGUGUGAUAUACUGCCA |
| | | | UCAGUCGGCGUGGACUGUAGAACACUGCCAAUGCCGGU |
| | | | CCCAAGCCCGGAUAAAAGUGGAGGGUACAGUCCACGCU |
| | | | UUUUUU |
| 3107 | 134B | Circular 871_198.105 | GGCCAUCAGUCGCCGGUCCCAAGCCCGGAUAAAAUGGG |
| | | | AGGGGGCGGGAAACCGCCUAACCAUGCCGACUGAUGGC |
| | | | AGAUAAUCUGUUGGUUAUAAAUGACAUUUCCUCUGGCA |
| | | | ACUUCAGGUGCACGAAACCCUGGUGUGCCCUCUGAUCC |
| | | | AAGCAUCCCCAUUCUACAGUAGGACUGAGCACUGCCGA |
| | | | GCUGGGCAAUCUUGAGUCGGAUGGCAGCAUUGGGAUAC |
| | | | AGUGUGAAAAGCAGCACAUUGUGGGGUUUCAGGUAUCG |
| | | | GUAUAUAAUCAUGGCAUAUACUGCCAUCAGUCGGCGUG |
| | | | GACUGUAGAACACUGCCAAUGCCGGUCCCAAGCCCGGA |
| | | | UAAAAGUGGAGGGUACAGUCCACGCUUUUUUU |
| 3108 | 134B | Circular 871_198.105_4 | GGCCAUCAGUCGCCGGUCCCAAGCCCGGAUAAAAUGGG |
| | | | AGGGGGCGGGAAACCGCCUAACCAUGCCGACUGAUGGC |
| | | | AGAUAAUCUGUUGGUUAUAAAUGACAUUUCCUGACCGU |
| | | | ACUUCAGGUGCACGAUUGGGAGGUGUGCCCUCUGAUCC |
| | | | AAGCAUCCCCAUUCUACAGUAGGACUGAGCACUGCCGA |
| | | | GCUGGGCAAUCUUGAGUCGGAUGGCAGCAUUGGGUAUG |
| | | | UCUGUGAAAAGCAGCACUAACACGGGUUUCAGGUAUCG |
| | | | GUAUAUAAUCAUGGCAUAUACUGCCAUCAGUCGGCGUG |
| | | | GACUGUAGAACACUGCCAAUGCCGGUCCCAAGCCCGGA |
| | | | UAAAAGUGGAGGGUACAGUCCACGCUUUUUUU |
| 3109 | 134B | Circular 919_196.100 | GGCCAUCAGUCGCCGGUCCCAAGCCCGGAUAAAAUGGG |
| | | | AGGGGGCGGGAAACCGCCUAACCAUGCCGACUGAUGGC |
| | | | AGAUAAUGGUUAUAAAUGACAUUUCCUCUGGCAACUUC |
| | | | AGGUGCACGAAACCCUGGUGUGCCCUCUGAUGUUGUUA |
| | | | UCCGGCGAGUACAGCAGUUCGGAGGAAUCCCGAGGUCA |
| | | | GCAAUCCUAAGUAUGAUGGCAGCAUUGGGAUACAGUGU |
| | | | GAAAAGCAGCACAUUGUGGGGUUUCAGGUAUCGGUAUA |
| | | | UAAUCAUGGCUGAAUAUACUGCCAUCAGUCGGCGUGGA |
| | | | CUGUAGAACACUGCCAAUGCCGGUCCCAAGCCCGGAUA |
| | | | AAAGUGGAGGGUACAGUCCACGCUUUUUUU |
| 3110 | 134B | Circular 919_196.100_4 | GGCCAUCAGUCGCCGGUCCCAAGCCCGGAUAAAAUGGG |
| | | | AGGGGGCGGGAAACCGCCUAACCAUGCCGACUGAUGGC |
| | | | AGAUAAUGGUUAUAAAUGACAUUUCCUCUGGCAACUUG |
| | | | UCCACCACGAAACCCUGGUGACGGGACUGAUGUUGUUA |
| | | | UCCGGCGAGUACAGCAGUUCGGAGGAAUCCCGAGGUCA |
| | | | GCAAUCCUAAGUAUGAUGGCAGCAUUGCCUAUGAGUGU |
| | | | GAAAAGCAGCUGUAACUGGGGUUUCAGGUAUCGGUAUA |
| | | | UAAUCAUGGCUGAAUAUACUGCCAUCAGUCGGCCUGGA |
| | | | CUGUAGAACACUGCCAAUGCCGGUCCCAAGCCCGGAUA |
| | | | AAAGUGGAGGGUACAGUCCACGCUUUUUUU |
| 3111 | 134A | Circular 871_128.70 | GGCCAUCAGUCGCCGGUCCCAAGCCCGGAUAAAAUGGG |
| | | | AGGGGGCGGGAAACCGCCUAACCAUGCCGACUGAUGGC |
| | | | AGAUAAUCAGGUGCACGAAACCCUGGUGUGCCCUCUGA |
| | | | UCCAAGCAUCCCCAUUCUACAGUAGGACUGAGCACUGC |
| | | | CGAGCUGGGCAAUCUUGAGUCGGAUGGCAGCAUUGGGA |
| | | | UACAGUGUGAAAAGCAGCACAAUAUACUGCCAUCAGUC |
| | | | GGCGUGGACUGUAGAACACUGCCAAUGCCGGUCCCAAG |
| | | | CCCGGAUAAAAGUGGAGGGUACAGUCCACGCUUUUUUU |
| 3112 | 134A | Circular 871_158.85 | GGCCAUCAGUCGCCGGUCCCAAGCCCGGAUAAAAUGGG |
| | | | AGGGGGCGGGAAACCGCCUAACCAUGCCGACUGAUGGC |
| | | | AGAUAAUUUCCUCUGGCAACUUCAGGUGCACGAAACCC |
| | | | UGGUGUGCCCUCUGAUCCAAGCAUCCCCAUUCUACAGU |
| | | | AGGACUGAGCACUGCCGAGCUGGGCAAUCUUGAGUCGG |
| | | | AUGGCAGCAUUGGGAUACAGUGUGAAAAGCAGCACAUU |
| | | | GUGGGGUUUCAGGAUAUACUGCCAUCAGUCGGCGUGGA |
| | | | CUGUAGAACACUGCCAAUGCCGGUCCCAAGCCCGGAUA |
| | | | AAAGUGGAGGGUACAGUCCACGCUUUUUUU |
| 3113 | 134A | Circular 919_126.65 | GGCCAUCAGUCGCCGGUCCCAAGCCCGGAUAAAAUGGG |
| | | | AGGGGGCGGGAAACCGCCUAACCAUGCCGACUGAUGGC |
| | | | AGAUAAUGCACGAAACCCUGGUGUGCCCUCUGAUGUUG |
| | | | UUAUCCGGCGAGUACAGCAGUUCGGAGGAAUCCCGAGG |
| | | | UCAGCAAUCCUAAGUAUGAUGGCAGCAUUGGGAUACAG |
| | | | UGUGAAAAGCAGCACAUUGAUAUACUGCCAUCAGUCGG |
| | | | CGUGGACUGUAGAACACUGCCAAUGCCGGUCCCAAGCC |
| | | | CGGAUAAAAGUGGAGGGUACAGUCCACGCUUUUUUU |
| 3114 | 134A | Circular 919_156.80 | GGCCAUCAGUCGCCGGUCCCAAGCCCGGAUAAAAUGGG |
| | | | AGGGGGCGGGAAACCGCCUAACCAUGCCGACUGAUGGC |
| | | | AGAUAAUCUGGCAACUUCAGGUGCACGAAACCCUGGUG |
| | | | UGCCCUCUGAUGUUGUUAUCCGGCGAGUACAGCAGUUC |
| | | | GGAGGAAUCCCGAGGUCAGCAAUCCUAAGUAUGAUGGC |
| | | | AGCAUUGGGAUACAGUGUGAAAAGCAGCACAUUGUGGG |
| | | | GUUUCAGGUCUAUAUACUGCCAUCAGUCGGCGUGGACU |
| | | | GUAGAACACUGCCAAUGCCGGUCCCAAGCCCGGAUAAA |
| | | | AGUGGAGGGUACAGUCCACGCUUUUUUU |
| 3115 | 135 | Circular 919_196.100_2 | GGCCAUCAGUCGCCGGUCCCAAGCCCGGAUAAAAUGGG |
| | | | AGGGGGCGGGAAACCGCCUAACCAUGCCGACUGAUGGC |
| | | | AGAUAAUGGUUAUAAAUGACAUUUCCUCUGGCAACUUC |
| | | | AGGUGCACGAAACCCUGGUGACGGGACUGAUGUUGUUA |
| | | | UCCGGCGAGUACAGCAGUUCGGAGGAAUCCCGAGGUCA |
| | | | GCAAUCCUAAGUAUGAUGGCAGCAUUGCCUAUGAGUGU |
| | | | GAAAAGCAGCACAUUGUGGGGUUUCAGGUAUCGGUAUA |
| | | | UAAUCAUGGCUGAAUAUACUGCCAUCAGUCGGCGUGGA |
| | | | CUGUAGAACACUGCCAAUGCCGGUCCCAAGCCCGGAUA |
| | | | AAAGUGGAGGGUACAGUCCACGCUUUUUUU |
| 3116 | 135 | Circular | GGCCAUCAGUCGCCGGUCCCAAGCCCGGAUAAAAUGGG |
| | | 919_196.100_2_U- | AGGGGGGGGGAAACCGCCUAACCAUGCCGACUGAUGGC |
| | | deletions | AGAUAAUGGUUAUAAAUGACAUUUCCUCUGGCAACCAG |
| | | | GUGCACGAAACCCUGGUGACGGGACUGAUGUUGUUAUC |
| | | | CGGCGAGUACAGCAGUUCGGAGGAAUCCCGAGGUCAGC |
| | | | AAUCCUAAGUAUGAUGGCAGCAUUGCCUAUGAGUGUGA |
| | | | AAAGCAGCACAUUGUGGGGCAGGAUCGGAUAAAUCAUG |
| | | | GCUGAAUAUACUGCCAUCAGUCGGCGUGGACUGUAGAA |
| | | | CACUGCCAAUGCCGGUCCCAAGCCCGGAUAAAAGUGGA |
| | | | GGGUACAGUCCACGCUUUUUUU |

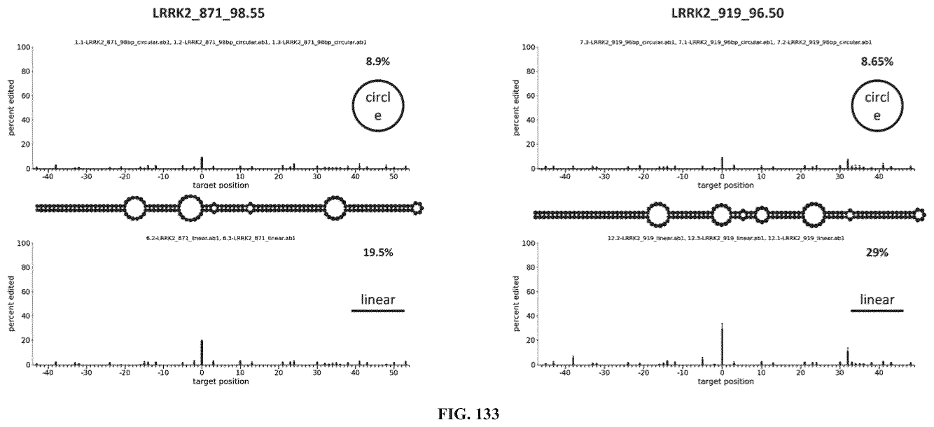

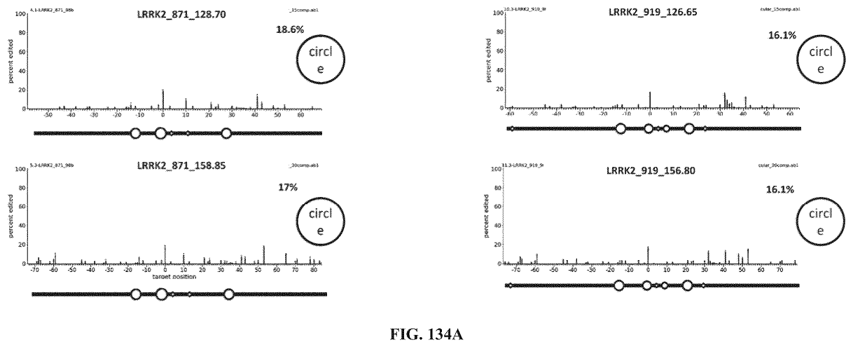

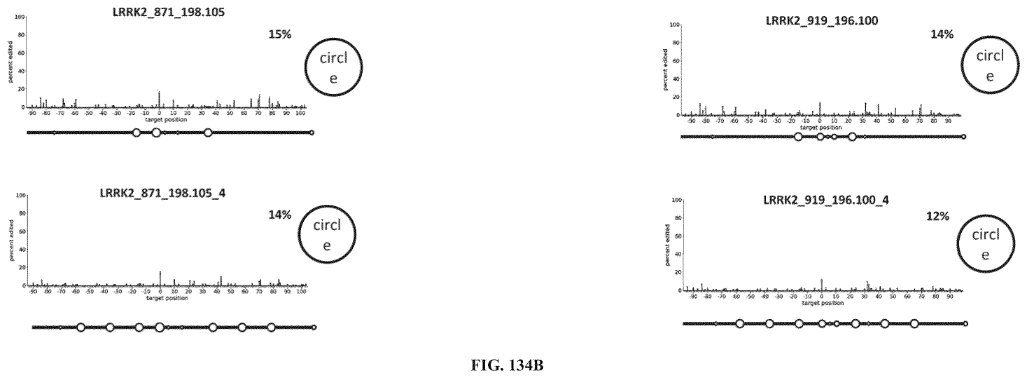

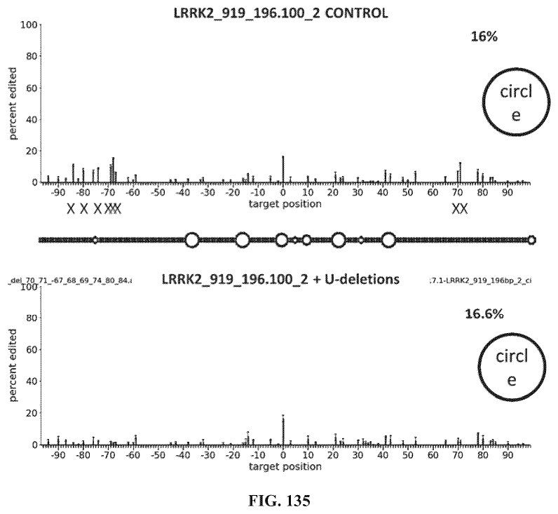

FIG. 133 provides a comparison between linear and circularized versions of exemplary guide RNAs guide871 and guide919 targeting LRRK2. While the editing efficiency of the circularized versions of the guide RNAs were lower than the linear counterparts, editing efficiency was increased by lengthening the circularized guide RNAs by an additional 15 nucleotides (FIG. 134A), 30 nucleotides (FIG. 134A), and 100 nucleotides (FIG. 134B). Finally, selected uridines were deleted from the circularized guide 919 to produce a U-deletion variant, and the effect of the U deletions on editing is depicted in FIG. 135.

### Example 33

### In Vivo Efficacy of AAV Vector Encoding an Engineered Guide RNA Targeting LRRK2

The scAAV vector constructed in EXAMPLE 23 was utilized in this example. Following QC validation of on-target LRRK2 G2019S editing and guide expression, the scAAV vector was packaged into scAAVDJ virus for in vivo testing in LRRK2 G2019S transgenic mice.

### Experimental Animals

Hemizygous BAC LRRK2 G2019S transgenic mice were utilized for this example (C57BL/6J-Tg(LRRK2*G2019S)2AMjff/J; Strain #018785).

### Thy1.1 Enrichment

Brain (ICV) and liver (IV) tissue samples were dissected from experimental mice and dissociated into single-cell suspensions using gentleMACS Dissociator (Miltenyi Biotec). Following dissociation, an aliquot of each sample was set aside and designated as “Pre-Enrichment”. The remainder of the samples were enriched for Thy1.1-expressing cells using CD90.1 MicroBeads (Miltenyi Biotec; Cat #130-121-273) by MACS and designated as “Post-Enrichment”.

### Sanger Sequencing/EditADAR Analysis

RNA extraction of “Pre-enrichment” and “Post-enrichment” brain and liver samples was performed using mirVana™ miRNA isolation kit (ThermoFisher) per manufacturer protocol. Synthesis of cDNA was performed using ProtoScript® II First Strand cDNA synthesis kit (NEB) per manufacturer protocol. PCR amplification of the LRRK2 G2019S target locus was performed using Q5@ High-Fidelity 2× master mix (NEB) using the following specific primers and thermocycler settings:

| Primer | Sequence |
|---|---|
| hLRRK2 | acaaagccagcctcactaga (SEQ ID NO: 3117) |
| 6020F | |
| hLRRK2 | tcaaagacctgggcagaagt (SEQ ID NO: 3118) |
| 6524R | |
| Thermo- | 98° C. - 30 s |
| cycler | [35 cycles] 98° C. - 10s, 67º C. - 30s, |
| Settings | 72° C. - 20 s |
| | 72° C. -2 min |
| | 4º C. - hold |

Following PCR amplification and gel confirmation of specific product, ExoSAP-IT™ PCR product cleanup reagent (ThermoFisher) was added to samples prior to Sanger sequencing submission. Sequencing trace files (.ab1) were analyzed via EditADAR (ShapeTX) to generate RNA editing profiles.

ddPCR Guide RNA Quantitation

cDNA synthesis of “Pre-enrichment” and “Post-enrichment” brain and liver RNA samples was performed using ProtoScript® II First Strand cDNA synthesis kit (NEB) per manufacturer protocol with the following modification—use of smOPT specific primer (5′-CAGAAAACCTGCTCCAAAAATTCCAC-3′) with oligo d(T)23 VN at 1:1 ratio. ddPCR was performed on the QX200 system (Bio-Rad) using ddPCR Supermix for Probes (No dUTP) (Bio-Rad; Cat #1863024) using the following specific ddPCR primers and probes and thermocycler settings:

| Primer / | |
|---|---|
| Probe | Sequence |
| (F) | GTGTGCCCTCTGATGTTTTTC (SEQ ID NO: 3119) |
| (R) | CCCAATGCTTCGCTTATTGC (SEQ ID NO: 3120) |
| (Probe) | /56FAM/ACAACAGTA/ZEN/CTGAGCTATCCCG/ |
| | 3IABKFQ/ (SEQ ID NO: 3121) |
| GAPDH (F) | TGTAGTTGAGGTCAATGAAGGG (SEQ ID NO: |
| | 3122) |
| GAPDH (R) | ACATCGCTCAGACACCATG (SEQ ID NO: 3123) |
| GAPDH | /5HEX/AAGGTCGGA/ZEN/GTCAACGGATTTGGTC/ |
| (Probe) | 3IABKFQ/ (SEQ ID NO: 3124) |
| Thermo- | 95° C. - 10 min |
| cycler | [40 cycles] 94° C. - 30 s (2° C./s), |
| Settings | 60° C. - 60 s (2° C./s), 72° C. - 15 s |
| | (2° C./s) |
| | 98° C. - 10 min |
| | 98° C. - 10 min |
| | 4° C. - hold |

Absolute ddPCR values for the engineered guide RNA and GAPDH were recorded as copies/uL. Guide RNA copy numbers were normalized to GAPDH copy numbers.

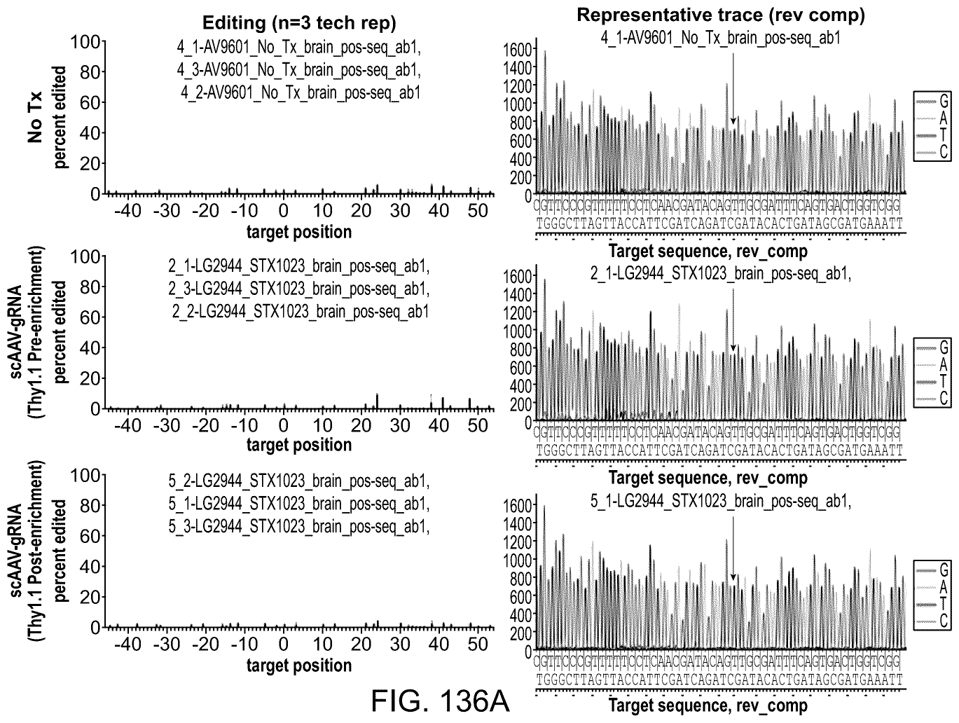

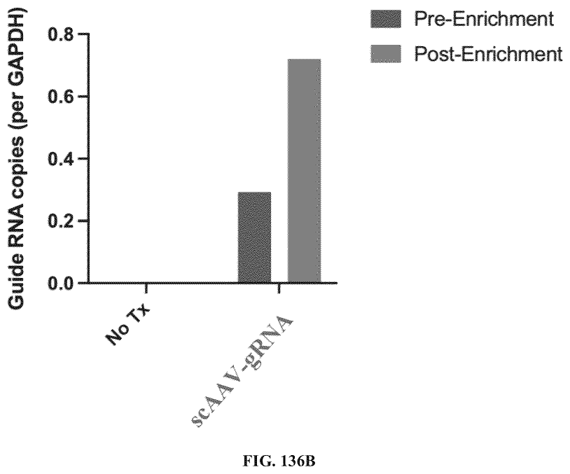

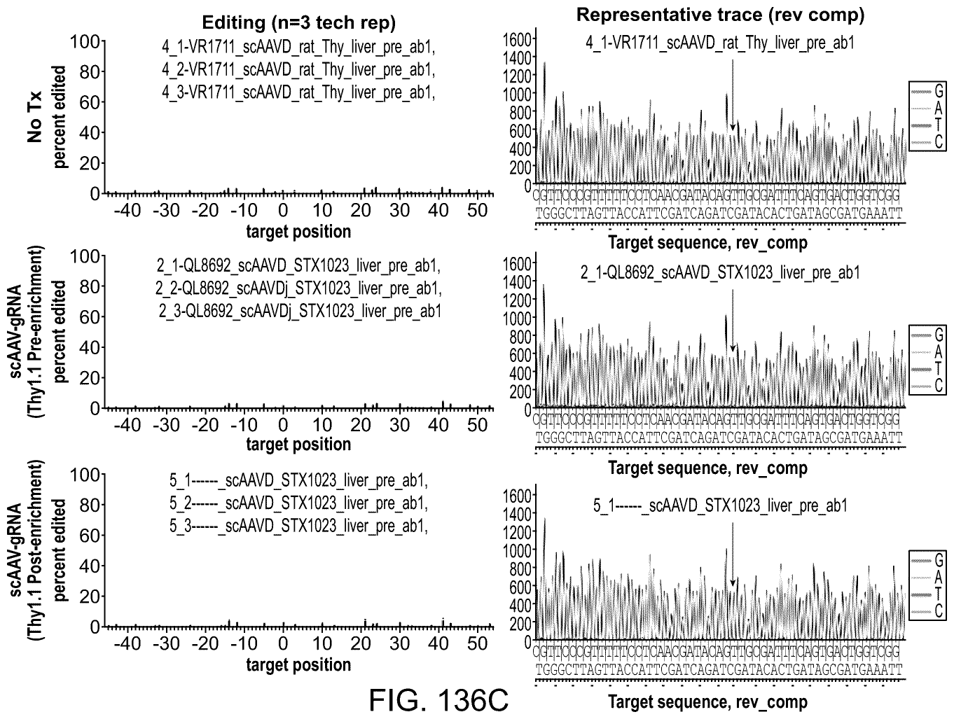

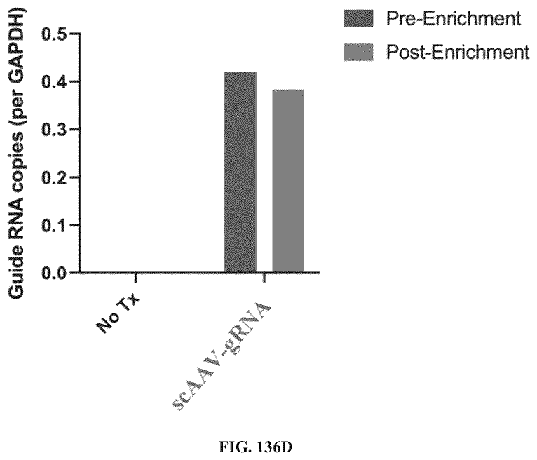

FIG. 136A and FIG. 136C depict the in vivo editing efficiencies for the scAAV vector encoding the engineered guide RNA targeting LRRK2, as measured in the brain (FIG. 136A) and liver (FIG. 136C). No detectable editing observed in scAAVDJ-engineered guide RNA ICV-injected mouse brain or liver tissue. EditADAR analysis and representative Sanger sequencing traces from no treatment, Thy1.1 pre-enrichment scAAVDJ-engineered guide RNA and Thy1.1 post-enrichment scAAVDJ-engineered guide RNA brain RNA samples. FIG. 136B and FIG. 136D illustrate quantitation of engineered guide RNA expression, as compared to expression of the GAPDH control, in the brain (FIG. 136B) and liver (FIG. 136D). Low levels of guide RNA expression (<1 guide RNA copy per GAPDH) in both Thy1.1 pre- and post-enrichment brain and liver samples as measured by ddPCR analysis was detected.

### Example 34

### Library Screen of SERPINA1 Targeting Guides

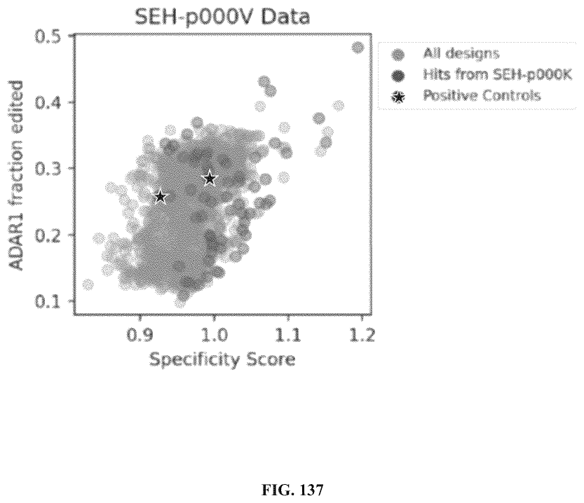

A library of 40 thousand short sequences (˜45 mers) targeting SERPINA1 was previously tested for in vitro editing of SERPINA1. The short sequences were grafted onto larger sequences with hairpins and/or internal loops for validation in targeting SERPINA1 and screened as a library. FIG. 137 shows the results of the library containing the short SERPINA1 targeting sequences grafted onto the larger sequences. The ADAR1 fraction edited is depicted on the Y-axis and the specificity score (taking into account only positions −9, −6, −2, 1, 3, 4, 9, 13) is depicted on the X-axis. The higher the specificity score, the lower the off-target; therefore, the more specific editing and highest ADAR 1 fraction edited designs are in the top right quadrant of the graph. The previous library of short sequences are shown in light gray. The hits from the larger grafted sequences are shown in the darker circles and the positive controls are indicated by stars.

### Exemplary Engineered Guide RNAS

The following TABLE 19 contains exemplary engineered guide RNAs utilized, for example, in the figures and examples provided in the present application. While the engineered guide RNA sequences in TABLE 19 are provided as DNA sequences with a T substituted for each U, the corresponding RNA sequences are also encompassed herein.

TABLE 19
Engineered Guide RNA Sequences

| | SEQ | | | | |
|---|---|---|---|---|---|
| | ID | Guide | | Structural Features | % RNA |
| FIG. | NO: | Name | Sequence | (target/guide) | Editing |
| FIG. 3 | 2 | 0.40.20 | AGCACTCTCCAGTGAGAACTC | 2/1 asymmetric bulge | 1.25 |
| | | | GGACCACAGCCTCCCGCTG | 1/0 asymmetric bulge | |
| | | | gtgg AATTTTTGGAG | 1 G/G mismatch | |
| | | | CAGGTTTTCTGACTTCGGTCGG | 1 A/C mismatch | |
| | | | AAAACCCCT | 3/3 symmetric bulge | |
| FIG. 3 | 3 | 0.45.25 | GCCAAAGCACTCTCCAGTGAG | 2/1 asymmetric bulge | 2.25 |
| | | | AACTCGGACCACAGCCTCCCG | 1/0 asymmetric bulge | |
| | | | CTG gtgg AATTTTTGGAG | 1 G/G mismatch | |
| | | | CAGGTTTTCTGACTTCGGTCGG | 1 A/C mismatch | |
| | | | AAAACCCCT | 3/3 symmetric bulge | |
| FIG. 3 | 4 | 0.50.30 | AGGAGGCCAAAGCACTCTCCA | 2/1 asymmetric bulge | 1.75 |
| | | | GTGAGAACTCGGACCACAGCC | 1/0 asymmetric bulge | |
| | | | TCCCGCTG gtgg | 1 G/G mismatch | |
| | | | AATTTTTGGAG | 1 A/C mismatch | |
| | | | CAGGTTTTCTGACTTCGGTCGG | 3/3 symmetric bulge | |
| | | | AAAACCCCT | | |
| FIG. 3 | 5 | 0.55.35 | CTCCCAGGAGGCCAAAGCACT | 2/1 asymmetric bulge | 2.75 |
| | | | CTCCAGTGAGAACTCGGACCA | 1/0 asymmetric bulge | |
| | | | CAGCCTCCCGCTG gtgg | 1 G/G mismatch | |
| | | | AATTTTTGGAG | 1 A/C mismatch | |
| | | | CAGGTTTTCTGACTTCGGTCGG | 3/3 symmetric bulge | |
| | | | AAAACCCCT | | |
| FIG. 3 | 6 | 0.60.40 | ATTCACTCCCAGGAGGCCAAA | 2/1 asymmetric bulge | 3.5 |
| | | | GCACTCTCCAGTGAGAACTCG | 1/0 asymmetric bulge | |
| | | | GACCACAGCCTCCCGCTG gtgg | 1 G/G mismatch | |
| | | | AATTTTTGGAG | 1 A/C mismatch | |
| | | | CAGGTTTTCTGACTTCGGTCGG | 3/3 symmetric bulge | |
| | | | AAAACCCCT | | |
| FIG. 3 | 7 | 0.65.45 | GCACCATTCACTCCCAGGAGG | 2/1 asymmetric bulge | 6.5 |
| | | | CCAAAGCACTCTCCAGTGAGA | 1/0 asymmetric bulge | |
| | | | ACTCGGACCACAGCCTCCCGC | 1 G/G mismatch | |
| | | | TG gtgg AATTTTTGGAG | 1 A/C mismatch | |
| | | | CAGGTTTTCTGACTTCGGTCGG | 3/3 symmetric bulge | |
| | | | AAAACCCCT | | |
| FIG. 3 | 8 | 0.70.50 | TGCCGGCACCATTCACTCCCA | 2/1 asymmetric bulge | 5.75 |
| | | | GGAGGCCAAAGCACTCTCCAG | 1/0 asymmetric bulge | |
| | | | TGAGAACTCGGACCACAGCCT | 1 G/G mismatch | |
| | | | CCCGCTG gtgg AATTTTTGGAG | 1 A/C mismatch | |
| | | | CAGGTTTTCTGACTTCGGTCGG | 3/3 symmetric bulge | |
| | | | AAAACCCCT | | |
| FIG. 3 | 9 | 0.75.55 | TGTATTGCCGGCACCATTCACT | 2/1 asymmetric bulge | 7.25 |
| | | | CCCAGGAGGCCAAAGCACTCT | 1/0 asymmetric bulge | |
| | | | CCAGTGAGAACTCGGACCACA | 1 G/G mismatch | |
| | | | GCCTCCCGCTG gtgg | 1 A/C mismatch | |
| | | | AATTTTTGGAG | 3/3 symmetric bulge | |
| | | | CAGGTTTTCTGACTTCGGTCGG | | |
| | | | AAAACCCCT | | |
| FIG. 3 | 10 | 0.80.60 | GTGGTTGTATTGCCGGCACCA | 2/1 asymmetric bulge | 8.75 |
| | | | TTCACTCCCAGGAGGCCAAAG | 1/0 asymmetric bulge | |
| | | | CACTCTCCAGTGAGAACTCGG | 1 G/G mismatch | |
| | | | ACCACAGCCTCCCGCTG gtgg | 1 A/C mismatch | |
| | | | AATTTTTGGAG | 3/3 symmetric bulge | |
| | | | CAGGTTTTCTGACTTCGGTCGG | | |
| | | | AAAACCCCT | | |
| FIG. 3 | 11 | 0.85.65 | TGAATGTGGTTGTATTGCCGG | 2/1 asymmetric bulge | 14 |
| | | | CACCATTCACTCCCAGGAGGC | 1/0 asymmetric bulge | |
| | | | CAAAGCACTCTCCAGTGAGAA | 1 G/G mismatch | |
| | | | CTCGGACCACAGCCTCCCGCT | 1 A/C mismatch | |
| | | | G gtgg AATTTTTGGAG | 3/3 symmetric bulge | |
| | | | CAGGTTTTCTGACTTCGGTCGG | | |
| | | | AAAACCCCT | | |
| FIG. 3 | 12 | 0.90.70 | CATCTTGAATGTGGTTGTATTG | 2/1 asymmetric bulge | 13.5 |
| | | | CCGGCACCATTCACTCCCAGG | 1/0 asymmetric bulge | |
| | | | AGGCCAAAGCACTCTCCAGTG | 1 G/G mismatch | |
| | | | AGAACTCGGACCACAGCCTCC | 1 A/C mismatch | |
| | | | CGCTG gtgg AATTTTTGGAG | 3/3 symmetric bulge | |
| | | | CAGGTTTTCTGACTTCGGTCGG | | |
| | | | AAAACCCCT | | |
| FIG. 3 | 13 | 0.95.75 | GTGAGCATCTTGAATGTGGTT | 2/1 asymmetric bulge | 10.25 |
| | | | GTATTGCCGGCACCATTCACT | 1/0 asymmetric bulge | |
| | | | CCCAGGAGGCCAAAGCACTCT | 1 G/G mismatch | |
| | | | CCAGTGAGAACTCGGACCACA | 1 A/C mismatch | |
| | | | GCCTCCCGCTG gtgg | 3/3 symmetric bulge | |
| | | | AATTTTTGGAG | | |
| | | | CAGGTTTTCTGACTTCGGTCGG | | |
| | | | AAAACCCCT | | |
| FIG. 3 | 14 | 0.100.80 | CCCCAGTGAGCATCTTGAATG | 2/1 asymmetric bulge | 9.5 |
| FIG. 20 | | | TGGTTGTATTGCCGGCACCATT | 1/0 asymmetric bulge | |
| | | | CACTCCCAGGAGGCCAAAGCA | 1 G/G mismatch | |
| | | | CTCTCCAGTGAGAACTCGGAC | 1 A/C mismatch | |
| | | | CACAGCCTCCCGCTG gtgg | 3/3 symmetric bulge | |
| | | | AATTTTTGGAG | | |
| | | | CAGGTTTTCTGACTTCGGTCGG | | |
| | | | AAAACCCCT | | |
| FIG. 4 | 15 | 0.85.65 | TGAATGTGGTTGTATTGCCGG | 2/1 asymmetric bulge | 3.5 |
| | | (−5, +39) | GTGGTATCACTCCCAGGAGGC | 4/4 symmetric bulge | |
| | | | CAAAGCACTCTCCAGTGAGAA | 1/0 asymmetric bulge | |
| | | | CTCGGACTGTTGGCTGTCGCT | 1 G/G mismatch | |
| | | | G gtgg AATTTTTGGAG | 1 A/C mismatch | |
| | | | CAGGTTTTCTGACTTCGGTCGG | 3/3 symmetric bulge | |
| | | | AAAACCCCT | 6/6 symmetric loop | |
| FIG. 4 | 16 | 0.86.65 | TGAATGTGGTTGTATTGCCGG | 2/1 asymmetric bulge | 9.6 |
| | | (−5, +36) | CACGTAAGTCTCCCAGGAGGC | 4/4 symmetric bulge | |
| | | | CAAAGCACTCTCCAGTGAGAA | 1/0 asymmetric bulge | |
| | | | CTCGGACTGTTGGCTGTCGCT | 1 G/G mismatch | |
| | | | G gtgg AATTTTTGGAG | 1 A/C mismatch | |
| | | | CAGGTTTTCTGACTTCGGTCGG | 3/3 symmetric bulge | |
| | | | AAAACCCCT | 6/6 symmetric loop | |
| FIG. 4 | 17 | 0.85.65 | TGAATGTGGTTGTATTGCCGG | 2/1 asymmetric bulge | 33.7 |
| FIG. 5 | | (−5, +33) | CACCATAGTGAGCCAGGAGGC | 4/4 symmetric bulge | |
| | | | CAAAGCACTCTCCAGTGAGAA | 1/0 asymmetric bulge | |
| | | | CTCGGACTGTTGGCTGTCGCT | 1 G/G mismatch | |
| | | | G gtgg AATTTTTGGAG | 1 A/C mismatch | |
| | | | CAGGTTTTCTGACTTCGGTCGG | 3/3 symmetric bulge | |
| | | | AAAACCCCT | 6/6 symmetric loop | |
| FIG. 4 | 18 | 0.85.65 | TGAATGTGGTTGTATTGCCGG | 2/1 asymmetric bulge | 13.2 |
| | | (−5, +30) | CACCATTCAGAGGGTGGAGGC | 4/4 symmetric bulge | |
| | | | CAAAGCACTCTCCAGTGAGAA | 1/0 asymmetric bulge | |
| | | | CTCGGACTGTTGGCTGTCGCT | 1 G/G mismatch | |
| | | | G gtgg AATTTTTGGAG | 1 A/C mismatch | |
| | | | CAGGTTTTCTGACTTCGGTCGG | 3/3 symmetric bulge | |
| | | | AAAACCCCT | 6/6 symmetric loop | |
| FIG. 4 | 19 | 0.85.65 | TGAATGTGGTTGTATTGCCGG | 2/1 asymmetric bulge | 22.8 |
| FIG. 5 | | (−5, +27) | CACCATTCACTCGGTCCTGGC | 4/4 symmetric bulge | |
| | | | CAAAGCACTCTCCAGTGAGAA | 1/0 asymmetric bulge | |
| | | | CTCGGACTGTTGGCTGTCGCT | 1 G/G mismatch | |
| | | | G gtgg AATTTTTGGAG | 1 A/C mismatch | |
| | | | CAGGTTTTCTGACTTCGGTCGG | 3/3 symmetric bulge | |
| | | | AAAACCCCT | 6/6 symmetric loop | |
| FIG. 4 | 20 | 0.85.65 | TGAATGTGGTTGTATTGCCGG | 2/1 asymmetric bulge | 15.6 |
| | | (−5, +24) | CACCATTCACTCCCACCTCCGC | 4/4 symmetric bulge | |
| | | | AAAGCACTCTCCAGTGAGAAC | 1/0 asymmetric bulge | |
| | | | TCGGACTGTTGGCTGTCGCTG | 1 G/G mismatch | |
| | | | gtgg AATTTTTGGAG | 1 A/C mismatch | |
| | | | CAGGTTTTCTGACTTCGGTCGG | 3/3 symmetric bulge | |
| | | | AAAACCCCT | 6/6 symmetric loop | |
| FIG. 4 | 21 | 0.85.65 | TGAATGTGGTTGTATTGCCGG | 2/1 asymmetric bulge | 9.5 |
| | | (−5, +20) | CACCATTCACTCCCAGGAGCG | 4/4 symmetric bulge | |
| | | | GTTTGCACTCTCCAGTGAGAA | 1/0 asymmetric bulge | |
| | | | CTCGGACTGTTGGCTGTCGCT | 1 G/G mismatch | |
| | | | G gtgg AATTTTTGGAG | 1 A/C mismatch | |
| | | | CAGGTTTTCTGACTTCGGTCGG | 3/3 symmetric bulge | |
| | | | AAAACCCCT | 6/6 symmetric loop | |
| FIG. 4 | 22 | 0.85.65 | TGAATGTGGTTGTATTGCCGG | 2/1 asymmetric bulge | 2.9 |
| | | (−5, +17) | CACCATTCACTCCCAGGAGGC | 4/4 symmetric bulge | |
| | | | CTTTCGTCTCTCCAGTGAGAAC | 1/0 asymmetric bulge | |
| | | | TCGGACTGTTGGCTGTCGCTG | 1 G/G mismatch | |
| | | | gtgg AATTTTTGGAG | 1 A/C mismatch | |
| | | | CAGGTTTTCTGACTTCGGTCGG | 3/3 symmetric bulge | |
| | | | AAAACCCCT | 6/6 symmetric loop | |
| FIG. 4 | 23 | 0.85.65 | TGAATGTGGTTGTATTGCCGG | 2/1 asymmetric bulge | 15.2 |
| FIG. 5 | | | CACCATTCACTCCCAGGAGGC | 1/0 asymmetric bulge | |
| FIG. 7 | | | CAAAGCACTCTCCAGTGAGAA | 1 G/G mismatch | |
| | | | CTCGGACCACAGCCTCCCGCT | 1 A/C mismatch | |
| | | | G gtgg AATTTTTGGAG | 3/3 symmetric bulge | |
| | | | CAGGTTTTCTGACTTCGGTCGG | | |
| | | | AAAACCCCT | | |
| FIG. 6 | 24 | 0.85.65 | TGAATGTGGTTGTTTTGCCGGC | 8/8 symmetrical loop | 46.3 |
| FIG. 7 | | (−12, +33) | ACCATAGTGAGCCAGGAGGCC | 1 G/G mismatch | |
| | | | AAAGCACTCTCCAGTGAGAAC | 1 A/C mismatch | |
| | | | TCGGACACACAGCGACAGCAC | 3/3 symmetric bulge | |
| | | | gtgg AATTTTTGGAG | 6/6 symmetric loop | |
| | | | CAGGTTTTCTGACTTCGGTCGG | | |
| | | | AAAACCCCT | | |
| FIG. 6 | 25 | 0.85.65 | TGAATGTGGTTGTTTTGCCGGC | 5/5 symmetrical loop | 38 |
| FIG. 7 | | (−11, +33) | ACCATAGTGAGCCAGGAGGCC | 1 G/G mismatch | |
| | | | AAAGCACTCTCCAGTGAGAAC | 1 A/C mismatch | |
| | | | TCGGACACACAGGGACACCAC | 3/3 symmetric bulge | |
| | | | gtgg AATTTTTGGAG | 6/6 symmetric loop | |
| | | | CAGGTTTTCTGACTTCGGTCGG | | |
| | | | AAAACCCCT | | |
| FIG. 6 | 26 | 0.85.65 | TGAATGTGGTTGTTTTGCCGGC | 5/5 symmetrical loop | 49.7 |
| FIG. 7 | | (−10, +33) | ACCATAGTGAGCCAGGAGGCC | 1 G/G mismatch | |
| FIG. 8 | | | AAAGCACTCTCCAGTGAGAAC | 1 A/C mismatch | |
| | | | TCGGACACACACGGACTCCAC | 3/3 symmetric bulge | |
| | | | gtgg AATTTTTGGAG | 6/6 symmetric loop | |
| | | | CAGGTTTTCTGACTTCGGTCGG | | |
| | | | AAAACCCCT | | |
| FIG. 6 | 27 | 0.85.65 | TGAATGTGGTTGTTTTGCCGGC | 5/5 symmetrical loop | 50.2 |
| FIG. 7 | | (−9, +33) | ACCATAGTGAGCCAGGAGGCC | 1 G/G mismatch | |
| FIG. 8 | | | AAAGCACTCTCCAGTGAGAAC | 1 A/C mismatch | |
| | | | TCGGACACACTCGGAGTCCAC | 3/3 symmetric bulge | |
| | | | gtgg AATTTTTGGAG | 6/6 symmetric loop | |
| | | | CAGGTTTTCTGACTTCGGTCGG | | |
| | | | AAAACCCCT | | |
| FIG. 6 | 28 | 0.85.65 | TGAATGTGGTTGTTTTGCCGGC | 4/4 symmetrical loop | 1.3 |
| FIG. 7 | | (−8, +33) | ACCATAGTGAGCCAGGAGGCC | 1 G/G mismatch | |
| | | | AAAGCACTCTCCAGTGAGAAC | 1 A/C mismatch | |
| | | | TCGGACACATTCGGTGTCCAC | 3/3 symmetric bulge | |
| | | | gtgg AATTTTTGGAG | 6/6 symmetric loop | |
| | | | CAGGTTTTCTGACTTCGGTCGG | | |
| | | | AAAACCCCT | | |
| FIG. 6 | 29 | 0.85.65 | TGAATGTGGTTGTTTTGCCGGC | 5/5 symmetrical loop | 42 |
| FIG. 7 | | (−7, +33) | ACCATAGTGAGCCAGGAGGCC | 1 G/G mismatch | |
| | | | AAAGCACTCTCCAGTGAGAAC | 1 A/C mismatch | |
| | | | TCGGACACTTTCGCTGTCCAC | 3/3 symmetric bulge | |
| | | | gtgg AATTTTTGGAG | 6/6 symmetric loop | |
| | | | CAGGTTTTCTGACTTCGGTCGG | | |
| | | | AAAACCCCT | | |
| FIG. 6 | 30 | 0.85.65 | TGAATGTGGTTGTTTTGCCGGC | 5/5 symmetrical loop | 34 |
| FIG. 7 | | (−6, +33) | ACCATAGTGAGCCAGGAGGCC | 1 G/G mismatch | |
| | | | AAAGCACTCTCCAGTGAGAAC | 1 A/C mismatch | |
| | | | TCGGACAGTTTCCCTGTCCAC | 3/3 symmetric bulge | |
| | | | gtgg AATTTTTGGAG | 6/6 symmetric loop | |
| | | | CAGGTTTTCTGACTTCGGTCGG | | |
| | | | AAAACCCCT | | |
| FIG. 6 | 31 | 0.85.65 | TGAATGTGGTTGTTTTGCCGGC | 3/3 symmetrical loop | 40.5 |
| FIG. 7 | | (−5, +33) | ACCATAGTGAGCCAGGAGGCC | 1 G/G mismatch | |
| FIG. 8 | | Version 3 | AAAGCACTCTCCAGTGAGAAC | 1 A/C mismatch | |
| | | | TCGGACTGTTGGCCTGTCCAC | 3/3 symmetric bulge | |
| | | | gtgg AATTTTTGGAG | 6/6 symmetric loop | |
| | | | CAGGTTTTCTGACTTCGGTCGG | | |
| | | | AAAACCCCT | | |
| FIG. 6 | 32 | 0.85.65 | TGAATGTGGTTGTATTGCCGG | 3/3 symmetrical loop | 39 |
| FIG. 7 | | (−5, +33) | CACCATAGTGAGCCAGGAGGC | 1 G/G mismatch | |
| | | Version 2 | CAAAGCACTCTCCAGTGAGAA | 1 A/C mismatch | |
| | | | CTCGGACTGTTGGCCTGTCCA | 3/3 symmetric bulge | |
| | | | C gtgg AATTTTTGGAG | 6/6 symmetric loop | |
| | | | CAGGTTTTCTGACTTCGGTCGG | | |
| | | | AAAACCCCT | | |
| FIG. 6 | 33 | 0.85.65 | TGAATGTGGTTGTATTGCCGG | 1/0 asymmetric bulge | 31.9 |
| FIG. 7 | | (−5, +33) | CACCATAGTGAGCCAGGAGGC | 4/4 symmetrical loop | |
| | | | CAAAGCACTCTCCAGTGAGAA | 1 G/G mismatch | |
| | | | CTCGGACTGTTGGCTGTCGCT | 1 A/C mismatch | |
| | | | G gtgg AATTTTTGGAG | 3/3 symmetric bulge | |
| | | | CAGGTTTTCTGACTTCGGTCGG | 6/6 symmetric loop | |
| | | | AAAACCCCT | | |
| FIG. 9 | 34 | 0.100.9 | TGAGAACTCGGACCACAGCCT | 2/1 asymmetric bulge | 0.5 |
| | | | CCCGCTGCTGGGCTGGAGGTG | 1/0 asymmetric bulge | |
| | | | CCTGGATAAATCTTGGTTAGTT | 1 G/G mismatch | |
| | | | CATGTAGCCTTAAGATGTCAG | 1 A/C mismatch | |
| | | | TATTATTTCCACCAG gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 35 | 0.100.12 | CAGTGAGAACTCGGACCACAG | 2/1 asymmetric bulge | 1.6 |
| | | | CCTCCCGCTGCTGGGCTGGAG | 1/0 asymmetric bulge | |
| | | | GTGCCTGGATAAATCTTGGTT | 1 G/G mismatch | |
| | | | AGTTCATGTAGCCTTAAGATG | 1 A/C mismatch | |
| | | | TCAGTATTATTTCCAC gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 36 | 0.100.18 | ACTCTCCAGTGAGAACTCGGA | 2/1 asymmetric bulge | 0 |
| | | | CCACAGCCTCCCGCACTGCTG | 1/0 asymmetric bulge | |
| | | | GGCTGGAGGTGCCTGGATAAA | 1 G/G mismatch | |
| | | | TCTTGGTTAGTTCATGTAGCCT | 1 A/C mismatch | |
| | | | TAAGATGTCAGTATT gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 37 | 0.100.20 | GCACTCTCCAGTGAGAACTCG | 2/1 asymmetric bulge | 0 |
| | | | GACCACAGCCTCCCGCTGCTG | 1/0 asymmetric bulge | |
| | | | GGCTGGAGGTGCCTGGATAAA | 1 G/G mismatch | |
| | | | TCTTGGTTAGTTCATGTAGCCT | 1 A/C mismatch | |
| | | | TAAGATGTCAGTATT gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 38 | 0.100.23 | AAAGCACTCTCCAGTGAGAAC | 2/1 asymmetric bulge | 0 |
| | | | TCGGACCACAGCCTCCCGCAC | 1/0 asymmetric bulge | |
| | | | TGCTGGGCTGGAGGTGCCTGG | 1 G/G mismatch | |
| | | | ATAAATCTTGGTTAGTTCATGT | 1 A/C mismatch | |
| | | | AGCCTTAAGATGTCA gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 39 | 0.100.25 | CCAAAGCACTCTCCAGTGAGA | 2/1 asymmetric bulge | 0 |
| | | | ACTCGGACCACAGCCTCCCGC | 1/0 asymmetric bulge | |
| | | | ACTGCTGGGCTGGAGGTGCCT | 1 G/G mismatch | |
| | | | GGATAAATCTTGGTTAGTTCA | 1 A/C mismatch | |
| | | | TGTAGCCTTAAGATGT gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 40 | 0.100.28 | AGGCCAAAGCACTCTCCAGTG | 2/1 asymmetric bulge | 0 |
| | | | AGAACTCGGACCACAGCCTCC | 1/0 asymmetric bulge | |
| | | | CGCACTGCTGGGCTGGAGGTG | 1 G/G mismatch | |
| | | | CCTGGATAAATCTTGGTTAGTT | 1 A/C mismatch | |
| | | | CATGTAGCCTTAAGA gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 41 | 0.100.30 | GGAGGCCAAAGCACTCTCCAG | 2/1 asymmetric bulge | 0 |
| | | | TGAGAACTCGGACCACAGCCT | 1/0 asymmetric bulge | |
| | | | CCCGCTGCTGGGCTGGAGGTG | 1 G/G mismatch | |
| | | | CCTGGATAAATCTTGGTTAGTT | 1 A/C mismatch | |
| | | | CATGTAGCCTTAAGA gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 42 | 0.100.33 | CCAGGAGGCCAAAGCACTCTC | 2/1 asymmetric bulge | 0 |
| | | | CAGTGAGAACTCGGACCACAG | 1/0 asymmetric bulge | |
| | | | CCTCCCGCACTGCTGGGCTGG | 1 G/G mismatch | |
| | | | AGGTGCCTGGATAAATCTTGG | 1 A/C mismatch | |
| | | | TTAGTTCATGTAGCCT gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 43 | 0.100.35 | TCCCAGGAGGCCAAAGCACTC | 2/1 asymmetric bulge | 0 |
| | | | TCCAGTGAGAACTCGGACCAC | 1/0 asymmetric bulge | |
| | | | AGCCTCCCGCACTGCTGGGCT | 1 G/G mismatch | |
| | | | GGAGGTGCCTGGATAAATCTT | 1 A/C mismatch | |
| | | | GGTTAGTTCATGTAGC gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 44 | 0.100.37 | ACTCCCAGGAGGCCAAAGCAC | 2/1 asymmetric bulge | 12.9 |
| | | | TCTCCAGTGAGAACTCGGACC | 1/0 asymmetric bulge | |
| | | | ACAGCCTCCCGCACTGCTGGG | 1 G/G mismatch | |
| | | | CTGGAGGTGCCTGGATAAATC | 1 A/C mismatch | |
| | | | TTGGTTAGTTCATGTA gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 45 | 0.100.40 | TTCACTCCCAGGAGGCCAAAG | 2/1 asymmetric bulge | 8.6 |
| | | | CACTCTCCAGTGAGAACTCGG | 1/0 asymmetric bulge | |
| | | | ACCACAGCCTCCCGCTGCTGG | 1 G/G mismatch | |
| | | | GCTGGAGGTGCCTGGATAAAT | 1 A/C mismatch | |
| | | | CTTGGTTAGTTCATGT gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 46 | 0.100.45 | CACCATTCACTCCCAGGAGGC | 2/1 asymmetric bulge | 8.2 |
| | | | CAAAGCACTCTCCAGTGAGAA | 1/0 asymmetric bulge | |
| | | | CTCGGACCACAGCCTCCCGCT | 1 G/G mismatch | |
| | | | GCTGGGCTGGAGGTGCCTGGA | 1 A/C mismatch | |
| | | | TAAATCTTGGTTAGTT gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 47 | 0.100.50 | GCCGGCACCATTCACTCCCAG | 2/1 asymmetric bulge | 6.7 |
| | | | GAGGCCAAAGCACTCTCCAGT | 1/0 asymmetric bulge | |
| | | | GAGAACTCGGACCACAGCCTC | 1 G/G mismatch | |
| | | | CCGCTGCTGGGCTGGAGGTGC | 1 A/C mismatch | |
| | | | CTGGATAAATCTTGGT gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 48 | 0.100.55 | GTATTGCCGGCACCATTCACT | 2/1 asymmetric bulge | 5.5 |
| | | | CCCAGGAGGCCAAAGCACTCT | 1/0 asymmetric bulge | |
| | | | CCAGTGAGAACTCGGACCACA | 1 G/G mismatch | |
| | | | GCCTCCCGCTGCTGGGCTGGA | 1 A/C mismatch | |
| | | | GGTGCCTGGATAAATC gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 49 | 0.100.60 | TGGTTGTATTGCCGGCACCATT | 2/1 asymmetric bulge | 13.4 |
| | | | CACTCCCAGGAGGCCAAAGCA | 1/0 asymmetric bulge | |
| | | | CTCTCCAGTGAGAACTCGGAC | 1 G/G mismatch | |
| | | | CACAGCCTCCCGCTGCTGGGC | 1 A/C mismatch | |
| | | | TGGAGGTGCCTGGAT gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 50 | 0.100.65 | GAATGTGGTTGTATTGCCGGC | 2/1 asymmetric bulge | 22.1 |
| | | | ACCATTCACTCCCAGGAGGCC | 1/0 asymmetric bulge | |
| | | | AAAGCACTCTCCAGTGAGAAC | 1 G/G mismatch | |
| | | | TCGGACCACAGCCTCCCGCAC | 1 A/C mismatch | |
| | | | TGCTGGGCTGGAGGTG gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 51 | 0.100.67 | TTGAATGTGGTTGTATTGCCG | 2/1 asymmetric bulge | 21.7 |
| | | | GCACCATTCACTCCCAGGAGG | 1/0 asymmetric bulge | |
| | | | CCAAAGCACTCTCCAGTGAGA | 1 G/G mismatch | |
| | | | ACTCGGACCACAGCCTCCCGC | 1 A/C mismatch | |
| | | | ACTGCTGGGCTGGAGG gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 52 | 0.100.70 | ATCTTGAATGTGGTTGTATTGC | 2/1 asymmetric bulge | 26.7 |
| | | | CGGCACCATTCACTCCCAGGA | 1/0 asymmetric bulge | |
| | | | GGCCAAAGCACTCTCCAGTGA | 1 G/G mismatch | |
| | | | GAACTCGGACCACAGCCTCCC | 1 A/C mismatch | |
| | | | GCTGCTGGGCTGGAG gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 53 | 0.100.72 | GCATCTTGAATGTGGTTGTATT | 2/1 asymmetric bulge | 22.1 |
| | | | GCCGGCACCATTCACTCCCAG | 1/0 asymmetric bulge | |
| | | | GAGGCCAAAGCACTCTCCAGT | 1 G/G mismatch | |
| | | | GAGAACTCGGACCACAGCCTC | 1 A/C mismatch | |
| | | | CCGCACTGCTGGGCT gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 54 | 0.100.75 | TGAGCATCTTGAATGTGGTTG | 2/1 asymmetric bulge | 14.5 |
| | | | TATTGCCGGCACCATTCACTCC | 1/0 asymmetric bulge | |
| | | | CAGGAGGCCAAAGCACTCTCC | 1 G/G mismatch | |
| | | | AGTGAGAACTCGGACCACAGC | 1 A/C mismatch | |
| | | | CTCCCGCACTGCTGG gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 55 | 0.100.78 | CAGTGAGCATCTTGAATGTGG | 2/1 asymmetric bulge | 11.3 |
| | | | TTGTATTGCCGGCACCATTCAC | 1/0 asymmetric bulge | |
| | | | TCCCAGGAGGCCAAAGCACTC | 1 G/G mismatch | |
| | | | TCCAGTGAGAACTCGGACCAC | 1 A/C mismatch | |
| | | | AGCCTCCCGCACTGC gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 56 | 0.100.81 | CCCCAGTGAGCATCTTGAATG | 2/1 asymmetric bulge | 14 |
| | | | TGGTTGTATTGCCGGCACCATT | 1/0 asymmetric bulge | |
| | | | CACTCCCAGGAGGCCAAAGCA | 1 G/G mismatch | |
| | | | CTCTCCAGTGAGAACTCGGAC | 1 A/C mismatch | |
| | | | CACAGCCTCCCGCTG gtgg | 3/3 symmetric bulge | |
| | | | AATTTTTGGAG | | |
| | | | CAGGTTTTCTGACTTCGGTCGG | | |
| | | | AAAACCCCT | | |
| FIG. 9 | 57 | 0.100.84 | TGTCCCCAGTGAGCATCTTGA | 2/1 asymmetric bulge | 7.9 |
| | | | ATGTGGTTGTATTGCCGGCAC | 1/0 asymmetric bulge | |
| | | | CATTCACTCCCAGGAGGCCAA | 1 G/G mismatch | |
| | | | AGCACTCTCCAGTGAGAACTC | 1 A/C mismatch | |
| | | | GGACCACAGCCTCCCG gtgg | 3/3 symmetric bulge | |
| | | | AATTTTTGGAG | | |
| | | | CAGGTTTTCTGACTTCGGTCGG | | |
| | | | AAAACCCCT | | |
| FIG. 10 | 58 | 0.100.70 | ATCTTGAATGTGGTTGTATTGC | 2/1 asymmetric bulge | 3.8 |
| FIG. 11 | | (−5, +40) | CGCGTGGTTTCACTCCCAGGA | 6/6 symmetric loop | |
| | | | GGCCAAAGCACTCTCCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 59 | 0.100.70 | ATCTTGAATGTGGTTGTATTGC | 2/1 asymmetric bulge | 4.5 |
| FIG. 11 | | (−5, +38) | CGGCTGGTAACACTCCCAGGA | 6/6 symmetric loop | |
| | | | GGCCAAAGCACTCTCCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 60 | 0.100.70 | ATCTTGAATGTGGTTGTATTGC | 2/1 asymmetric bulge | 6 |
| FIG. 11 | | (−5, +36) | CGGCACGTAAGTCTCCCAGGA | 6/6 symmetric loop | |
| | | | GGCCAAAGCACTCTCCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 61 | 0.100.70 | ATCTTGAATGTGGTTGTATTGC | 2/1 asymmetric bulge | 17.9 |
| FIG. 11 | | (−5, +34) | CGGCACCAAAGTGACCCAGGA | 6/6 symmetric loop | |
| | | | GGCCAAAGCACTCTCCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 62 | 0.100.70 | ATCTTGAATGTGGTTGTATTGC | 2/1 asymmetric bulge | 23.7 |
| FIG. 11 | | (−5, +32) | CGGCACCATTGTGAGGCAGGA | 6/6 symmetric loop | |
| | | | GGCCAAAGCACTCTCCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 63 | 0.100.70 | ATCTTGAATGTGGTTGTATTGC | 2/1 asymmetric bulge | 10.8 |
| FIG. 11 | | (−5, +30) | CGGCACCATTCAGAGGGTGGA | 6/6 symmetric loop | |
| | | | GGCCAAAGCACTCTCCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 64 | 0.100.70 | ATCTTGAATGTGGTTGTATTGC | 2/1 asymmetric bulge | 9.8 |
| FIG. 11 | | (−5, +27) | CGGCACCATTCACTCGGTCCT | 6/6 symmetric loop | |
| | | | GGCCAAAGCACTCTCCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 65 | 0.100.70 | ATCTTGAATGTGGTTGTATTGC | 2/1 asymmetric bulge | 15 |
| FIG. 11 | | (−5, +25) | CGGCACCATTCACTCCCTCCTC | 6/6 symmetric loop | |
| | | | CCCAAAGCACTCTCCAGTGAG | 1 G/G mismatch | |
| | | | AACTCGGACTGTGTCCCTCCC | 1 A/C mismatch | |
| | | | GCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 66 | 0.100.70 | ATCTTGAATGTGGTTGTATTGC | 2/1 asymmetric bulge | 12.3 |
| FIG. 11 | | (−5, +23) | CGGCACCATTCACTCCCAGCT | 6/6 symmetric loop | |
| | | | CCGGAAAGCACTCTCCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 67 | 0.100.70 | ATCTTGAATGTGGTTGTATTGC | 2/1 asymmetric bulge | 17 |
| FIG. 11 | | (−5, +20) | CGGCACCATTCACTCCCAGGA | 6/6 symmetric loop | |
| | | | GCGGTTTGCACTCTCCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 68 | 0.100.70 | ATCTTGAATGTGGTTGTATTGC | 2/1 asymmetric bulge | 13.3 |
| FIG. 11 | | (−5, +18) | CGGCACCATTCACTCCCAGGA | 6/6 symmetric loop | |
| | | | GGCGTTTCGACTCTCCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 69 | 0.100.70 | ATCTTGAATGTGGTTGTATTGC | 2/1 asymmetric bulge | 2.5 |
| FIG. 11 | | (−5, +16) | CGGCACCATTCACTCCCAGGA | 6/6 symmetric loop | |
| | | | GGCCATTCGTGTCTCCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 70 | 0.100.70 | ATCTTGAATGTGGTTGTATTGC | 2/1 asymmetric bulge | 1 |
| FIG. 11 | | (−5, +14) | CGGCACCATTCACTCCCAGGA | 6/6 symmetric loop | |
| | | | GGCCAAACGTGAGTCCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 71 | 0.100.70 | ATCTTGAATGTGGTTGTATTGC | 2/1 asymmetric bulge | 1 |
| FIG. 11 | | (−5, +12) | CGGCACCATTCACTCCCAGGA | 6/6 symmetric loop | |
| | | | GGCCAAAGCTGAGAGCAGTGA | 1 G/G mismatch | |
| | | | GAACTCGGACTGTGTCCCTCC | 1 A/C mismatch | |
| | | | CGCTGCTGGGCTGGAgtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 10 | 72 | 0.100.70 | ATCTTGAATGTGGTTGTATTGC | 2/1 asymmetric bulge | 16.5 |
| FIG. 11 | | | CGGCACCATTCACTCCCAGGA | 1/0 asymmetric bulge | |
| FIG. 12 | | | GGCCAAAGCACTCTCCAGTGA | 1 G/G mismatch | |
| FIG. 13 | | | GAACTCGGACCACAGCCTCCC | 1 A/C mismatch | |
| FIG. 14 | | | GCTGCTGGGCTGGAG gtgg | 3/3 symmetric bulge | |
| | | | AATTTTTGGAG | | |
| | | | CAGGTTTTCTGACTTCGGTCGG | | |
| | | | AAAACCCCT | | |
| FIG. 12 | 73 | 0.100.70 | ATCTTGAATGTGGTTGTTTTGC | 6/6 symmetric loop | 48.8 |
| FIG. 13 | | (−15, +33) | CGGCACCATAGTGAGCCAGGA | 1 G/G mismatch | |
| FIG. 14 | | | GGCCAAAGCACTCTCCAGTGA | 1 A/C mismatch | |
| FIG. 18 | | | GAACTCGGACACACAGCCTGA | 3/3 symmetric bulge | |
| | | | GTGAGCTGGGCTGGA gtgg | 6/6 symmetric loop | |
| | | | AATTTTTGGAG | | |
| | | | CAGGTTTTCTGACTTCGGTCGG | | |
| | | | AAAACCCCT | | |
| FIG. 12 | 74 | 0.100.70 | ATCTTGAATGTGGTTGTTTTGC | 6/6 symmetric loop | 45.2 |
| FIG. 13 | | (−14, +33) | CGGCACCATAGTGAGCCAGGA | 1 G/G mismatch | |
| FIG. 14 | | | GGCCAAAGCACTCTCCAGTGA | 1 A/C mismatch | |
| | | | GAACTCGGACACACAGCCTCA | 3/3 symmetric bulge | |
| | | | GTGTGCTGGGCTGGA gtgg | 6/6 symmetric loop | |
| | | | AATTTTTGGAG | | |
| | | | CAGGTTTTCTGACTTCGGTCGG | | |
| | | | AAAACCCCT | | |
| FIG. 12 | 75 | 0.100.70 | ATCTTGAATGTGGTTGTTTTGC | 5/5 symmetric loop | 43 |
| FIG. 13 | | (−13, +33) | CGGCACCATAGTGAGCCAGGA | 1 G/G mismatch | |
| FIG. 14 | | | GGCCAAAGCACTCTCCAGTGA | 1 A/C mismatch | |
| | | | GAACTCGGACACACAGCCACA | 3/3 symmetric bulge | |
| | | | GACTGCTGGGCTGGA gtgg | 6/6 symmetric loop | |
| | | | AATTTTTGGAG | | |
| | | | CAGGTTTTCTGACTTCGGTCGG | | |
| | | | AAAACCCCT | | |
| FIG. 12 | 76 | 0.100.70 | ATCTTGAATGTGGTTGTTTTGC | 6/6 symmetric loop | 42.8 |
| FIG. 13 | | (−12, +33) | CGGCACCATAGTGAGCCAGGA | 1 G/G mismatch | |
| | | | GGCCAAAGCACTCTCCAGTGA | 1 A/C mismatch | |
| | | | GAACTCGGACACACAGCGACA | 3/3 symmetric bulge | |
| | | | GGCTGCTGGGCTGGA gtgg | 6/6 symmetric loop | |
| | | | AATTTTTGGAG | | |
| | | | CAGGTTTTCTGACTTCGGTCGG | | |
| | | | AAAACCCCT | | |
| FIG. 12 | 77 | 0.100.70 | ATCTTGAATGTGGTTGTTTTGC | 6/6 symmetric loop | 37 |
| FIG. 13 | | (−11, +33) | CGGCACCATAGTGAGCCAGGA | 1 G/G mismatch | |
| | | | GGCCAAAGCACTCTCCAGTGA | 1 A/C mismatch | |
| | | | GAACTCGGACACACAGGGACA | 3/3 symmetric bulge | |
| | | | CGCTGCTGGGCTGGA gtgg | 6/6 symmetric loop | |
| | | | AATTTTTGGAG | | |
| | | | CAGGTTTTCTGACTTCGGTCGG | | |
| | | | AAAACCCCT | | |
| FIG. 12 | 78 | 0.100.70 | ATCTTGAATGTGGTTGTTTTGC | 1/0 asymmetric bulge | 44.4 |
| FIG. 13 | | (−9, +33) | CGGCACCATAGTGAGCCAGGA | 5/5 symmetric loop | |
| FIG. 14 | | | GGCCAAAGCACTCTCCAGTGA | 1 G/G mismatch | |
| | | | GAACTCGGACACACTCGGAGT | 1 A/C mismatch | |
| | | | CGCTGCTGGGCTGGA gtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 12 | 79 | 0.100.70 | ATCTTGAATGTGGTTGTTTTGC | 1/0 asymmetric bulge | 34 |
| FIG. 13 | | (−6, +33) | CGGCACCATAGTGAGCCAGGA | 5/5 symmetric loop | |
| | | | GGCCAAAGCACTCTCCAGTGA | 1 G/G mismatch | |
| | | | GAACTCGGACAGTGTCCCTGT | 1 A/C mismatch | |
| | | | CGCTGCTGGGCTGGA gtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 12 | 80 | 0.100.70 | ATCTTGAATGTGGTTGTTTTGC | 1/0 asymmetric bulge | 33.7 |
| FIG. 13 | | (−5, +33) | CGGCACCATAGTGAGCCAGGA | 6/6 symmetric loop | |
| | | Version 2 | GGCCAAAGCACTCTCCAGTGA | 1 G/G mismatch | |
| | | | GAACTCGGACTGTGTCCCTGT | 1 A/C mismatch | |
| | | | CGCTGCTGGGCTGGA gtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 12 | 81 | 0.100.70 | ATCTTGAATGTGGTTGTATTGC | 2/1 asymmetric bulge | 24.4 |
| FIG. 13 | | (−5, +32) | CGGCACCATTGTGAGGCAGGA | 6/6 symmetric loop | |
| | | | GGCCAAAGCACTCTCCAGTGA | 1 G/G mismatch | |
| | | | GAACTCGGACTGTGTCCCTCC | 1 A/C mismatch | |
| | | | CGCTGCTGGGCTGGA gtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 15 | 82 | 0.78.48 | CGGCACCATAGTGAGCCAGGA | 1/0 asymmetric bulge | 6.6 |
| FIG. 16 | | (−9, +33) | GGCCAAAGCACTCTCCAGTGA | 5/5 symmetric loop | |
| | | | GAACTCGGACACACTCGGAGT | 1 G/G mismatch | |
| | | | CGCTGCTGGGCTGGA gtgg | 1 A/C mismatch | |
| | | | AATTTTTGGAG | 3/3 symmetric bulge | |
| | | | CAGGTTTTCTGACTTCGGTCGG | 6/6 symmetric loop | |
| | | | AAAACCCCT | | |
| FIG. 15 | 83 | 0.80.50 | GCCGGCACCATAGTGAGCCAG | 1/0 asymmetric bulge | 9.1 |
| FIG. 16 | | (−9, +33) | GAGGCCAAAGCACTCTCCAGT | 5/5 symmetric loop | |
| | | | GAGAACTCGGACACACTCGGA | 1 G/G mismatch | |
| | | | GTCGCTGCTGGGCTGGA gtgg | 1 A/C mismatch | |
| | | | AATTTTTGGAG | 3/3 symmetric bulge | |
| | | | CAGGTTTTCTGACTTCGGTCGG | 6/6 symmetric loop | |
| | | | AAAACCCCT | | |
| FIG. 15 | 84 | 0.82.52 | TTGCCGGCACCATAGTGAGCC | 1/0 asymmetric bulge | 16.8 |
| FIG. 16 | | (−9, +33) | AGGAGGCCAAAGCACTCTCCA | 5/5 symmetric loop | |
| | | | GTGAGAACTCGGACACACTCG | 1 G/G mismatch | |
| | | | GAGTCGCTGCTGGGCTGGA | 1 A/C mismatch | |
| | | | gtgg AATTTTTGGAG | 3/3 symmetric bulge | |
| | | | CAGGTTTTCTGACTTCGGTCGG | 6/6 symmetric loop | |
| | | | AAAACCCCT | | |
| FIG. 15 | 85 | 0.84.54 | TTTTGCCGGCACCATAGTGAG | 1/0 asymmetric bulge | 37.2 |
| FIG. 16 | | (−9, +33) | CCAGGAGGCCAAAGCACTCTC | 5/5 symmetric loop | |
| | | | CAGTGAGAACTCGGACACACT | 1 G/G mismatch | |
| | | | CGGAGTCGCTGCTGGGCTGGA | 1 A/C mismatch | |
| | | | gtgg AATTTTTGGAG | 3/3 symmetric bulge | |
| | | | CAGGTTTTCTGACTTCGGTCGG | 6/6 symmetric loop | |
| | | | AAAACCCCT | | |
| FIG. 15 | 86 | 0.86.56 | TGTTTTGCCGGCACCATAGTG | 1/0 asymmetric bulge | 37.5 |
| FIG. 16 | | (−9, +33) | AGCCAGGAGGCCAAAGCACTC | 5/5 symmetric loop | |
| | | | TCCAGTGAGAACTCGGACACA | 1 G/G mismatch | |
| | | | CTCGGAGTCGCTGCTGGGCTG | 1 A/C mismatch | |
| | | | GA gtgg AATTTTTGGAG | 3/3 symmetric bulge | |
| | | | CAGGTTTTCTGACTTCGGTCGG | 6/6 symmetric loop | |
| | | | AAAACCCCT | | |
| FIG. 15 | 87 | 0.88.58 | GTTGTTTTGCCGGCACCATAGT | 1/0 asymmetric bulge | 37.9 |
| FIG. 16 | | (−9, +33) | GAGCCAGGAGGCCAAAGCACT | 5/5 symmetric loop | |
| | | | CTCCAGTGAGAACTCGGACAC | 1 G/G mismatch | |
| | | | ACTCGGAGTCGCTGCTGGGCT | 1 A/C mismatch | |
| | | | GGA gtgg AATTTTTGGAG | 3/3 symmetric bulge | |
| | | | CAGGTTTTCTGACTTCGGTCGG | 6/6 symmetric loop | |
| | | | AAAACCCCT | | |
| FIG. 15 | 88 | 0.90.60 | TGGTTGTTTTGCCGGCACCATA | 1/0 asymmetric bulge | 37.3 |
| FIG. 16 | | (−9, +33) | GTGAGCCAGGAGGCCAAAGC | 5/5 symmetric loop | |
| | | | ACTCTCCAGTGAGAACTCGGA | 1 G/G mismatch | |
| | | | CACACTCGGAGTCGCTGCTGG | 1 A/C mismatch | |
| | | | GCTGGA gtgg AATTTTTGGAG | 3/3 symmetric bulge | |
| | | | CAGGTTTTCTGACTTCGGTCGG | 6/6 symmetric loop | |
| | | | AAAACCCCT | | |
| FIG. 15 | 89 | 0.92.62 | TGTGGTTGTTTTGCCGGCACCA | 1/0 asymmetric bulge | 46.6 |
| FIG. 16 | | (−9, +33) | TAGTGAGCCAGGAGGCCAAAG | 5/5 symmetric loop | |
| FIG. 17 | | | CACTCTCCAGTGAGAACTCGG | 1 G/G mismatch | |
| | | | ACACACTCGGAGTCGCTGCTG | 1 A/C mismatch | |
| | | | GGCTGGA gtgg AATTTTTGGAG | 3/3 symmetric bulge | |
| | | | CAGGTTTTCTGACTTCGGTCGG | 6/6 symmetric loop | |
| | | | AAAACCCCT | | |
| FIG. 15 | 90 | 0.94.64 | AATGTGGTTGTTTTGCCGGCA | 1/0 asymmetric bulge | 47.4 |
| FIG. 16 | | (−9, +33) | CCATAGTGAGCCAGGAGGCCA | 5/5 symmetric loop | |
| FIG. 17 | | | AAGCACTCTCCAGTGAGAACT | 1 G/G mismatch | |
| | | | CGGACACACTCGGAGTCGCTG | 1 A/C mismatch | |
| | | | CTGGGCTGGA gtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 15 | 91 | 0.96.66 | TGAATGTGGTTGTTTTGCCGGC | 1/0 asymmetric bulge | 46.1 |
| FIG. 16 | | (−9, +33) | ACCATAGTGAGCCAGGAGGCC | 5/5 symmetric loop | |
| FIG. 17 | | | AAAGCACTCTCCAGTGAGAAC | 1 G/G mismatch | |
| | | | TCGGACACACTCGGAGTCGCT | 1 A/C mismatch | |
| | | | GCTGGGCTGGA gtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 15 | 92 | 0.98.68 | CTTGAATGTGGTTGTTTTGCCG | 1/0 asymmetric bulge | 40.7 |
| FIG. 16 | | (−9, +33) | GCACCATAGTGAGCCAGGAGG | 5/5 symmetric loop | |
| | | | CCAAAGCACTCTCCAGTGAGA | 1 G/G mismatch | |
| | | | ACTCGGACACACTCGGAGTCG | 1 A/C mismatch | |
| | | | CTGCTGGGCTGGA gtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 15 | 93 | 0.100.70 | ATCTTGAATGTGGTTGTTTTGC | 1/0 asymmetric bulge | 41.2 |
| FIG. 16 | | (−9, +33) | CGGCACCATAGTGAGCCAGGA | 5/5 symmetric loop | |
| | | | GGCCAAAGCACTCTCCAGTGA | 1 G/G mismatch | |
| | | | GAACTCGGACACACTCGGAGT | 1 A/C mismatch | |
| | | | CGCTGCTGGGCTGGA gtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 18 | 94 | 0.78.48 | CGGCACCATAGTGAGCCAGGA | 6/6 symmetric loop | 7.1 |
| | | (−15, +33) | GGCCAAAGCACTCTCCAGTGA | 1 G/G mismatch | |
| | | | GAACTCGGACACACAGCCTGA | 1 A/C mismatch | |
| | | | GTGAGCTGGGCTGGA gtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 18 | 95 | 0.80.50 | GCCGGCACCATAGTGAGCCAG | 6/6 symmetric loop | 12.4 |
| | | (−15, +33) | GAGGCCAAAGCACTCTCCAGT | 1 G/G mismatch | |
| | | | GAGAACTCGGACACACAGCCT | 1 A/C mismatch | |
| | | | GAGTGAGCTGGGCTGGA gtgg | 3/3 symmetric bulge | |
| | | | AATTTTTGGAG | 6/6 symmetric loop | |
| | | | CAGGTTTTCTGACTTCGGTCGG | | |
| | | | AAAACCCCT | | |
| FIG. 18 | 96 | 0.82.52 | TTGCCGGCACCATAGTGAGCC | 6/6 symmetric loop | 19.8 |
| | | (−15, +33) | AGGAGGCCAAAGCACTCTCCA | 1 G/G mismatch | |
| | | | GTGAGAACTCGGACACACAGC | 1 A/C mismatch | |
| | | | CTGAGTGAGCTGGGCTGGA | 3/3 symmetric bulge | |
| | | | gtgg AATTTTTGGAG | 6/6 symmetric loop | |
| | | | CAGGTTTTCTGACTTCGGTCGG | | |
| | | | AAAACCCCT | | |
| FIG. 18 | 97 | 0.84.54 | TTTTGCCGGCACCATAGTGAG | 6/6 symmetric loop | 38.2 |
| | | (−15, +33) | CCAGGAGGCCAAAGCACTCTC | 1 G/G mismatch | |
| | | | CAGTGAGAACTCGGACACACA | 1 A/C mismatch | |
| | | | GCCTGAGTGAGCTGGGCTGGA | 3/3 symmetric bulge | |
| | | | gtgg AATTTTTGGAG | 6/6 symmetric loop | |
| | | | CAGGTTTTCTGACTTCGGTCGG | | |
| | | | AAAACCCCT | | |
| FIG. 18 | 98 | 0.86.56 | TGTTTTGCCGGCACCATAGTG | 6/6 symmetric loop | 41.9 |
| | | (−15, +33) | AGCCAGGAGGCCAAAGCACTC | 1 G/G mismatch | |
| | | | TCCAGTGAGAACTCGGACACA | 1 A/C mismatch | |
| | | | CAGCCTGAGTGAGCTGGGCTG | 3/3 symmetric bulge | |
| | | | GA gtgg AATTTTTGGAG | 6/6 symmetric loop | |
| | | | CAGGTTTTCTGACTTCGGTCGG | | |
| | | | AAAACCCCT | | |
| FIG. 18 | 99 | 0.88.58 | GTTGTTTTGCCGGCACCATAGT | 6/6 symmetric loop | 37.8 |
| | | (−15, +33) | GAGCCAGGAGGCCAAAGCACT | 1 G/G mismatch | |
| | | | CTCCAGTGAGAACTCGGACAC | 1 A/C mismatch | |
| | | | ACAGCCTGAGTGAGCTGGGCT | 3/3 symmetric bulge | |
| | | | GGA gtgg AATTTTTGGAG | 6/6 symmetric loop | |
| | | | CAGGTTTTCTGACTTCGGTCGG | | |
| | | | AAAACCCCT | | |
| FIG. 18 | 100 | 0.90.60 | TGGTTGTTTTGCCGGCACCATA | 6/6 symmetric loop | 50.7 |
| FIG. 19 | | (−15, +33) | GTGAGCCAGGAGGCCAAAGC | 1 G/G mismatch | |
| | | | ACTCTCCAGTGAGAACTCGGA | 1 A/C mismatch | |
| | | | CACACAGCCTGAGTGAGCTGG | 3/3 symmetric bulge | |
| | | | GCTGGA gtgg AATTTTTGGAG | 6/6 symmetric loop | |
| | | | CAGGTTTTCTGACTTCGGTCGG | | |
| | | | AAAACCCCT | | |
| FIG. 18 | 101 | 0.92.62 | TGTGGTTGTTTTGCCGGCACCA | 6/6 symmetric loop | 53 |
| FIG. 19 | | (−15, +33) | TAGTGAGCCAGGAGGCCAAAG | 1 G/G mismatch | |
| FIG. 20 | | | CACTCTCCAGTGAGAACTCGG | 1 A/C mismatch | |
| | | | ACACACAGCCTGAGTGAGCTG | 3/3 symmetric bulge | |
| | | | GGCTGGA gtgg AATTTTTGGAG | 6/6 symmetric loop | |
| | | | CAGGTTTTCTGACTTCGGTCGG | | |
| | | | AAAACCCCT | | |
| FIG. 18 | 102 | 0.94.64 | AATGTGGTTGTTTTGCCGGCA | 6/6 symmetric loop | 53.1 |
| FIG. 19 | | (−15, +33) | CCATAGTGAGCCAGGAGGCCA | 1 G/G mismatch | |
| | | | AAGCACTCTCCAGTGAGAACT | 1 A/C mismatch | |
| | | | CGGACACACAGCCTGAGTGAG | 3/3 symmetric bulge | |
| | | | CTGGGCTGGA gtgg | 6/6 symmetric loop | |
| | | | AATTTTTGGAG | | |
| | | | CAGGTTTTCTGACTTCGGTCGG | | |
| | | | AAAACCCCT | | |
| FIG. 18 | 103 | 0.96.66 | TGAATGTGGTTGTTTTGCCGGC | 6/6 symmetric loop | 49.4 |
| | | (−15, +33) | ACCATAGTGAGCCAGGAGGCC | 1 G/G mismatch | |
| | | | AAAGCACTCTCCAGTGAGAAC | 1 A/C mismatch | |
| | | | TCGGACACACAGCCTGAGTGA | 3/3 symmetric bulge | |
| | | | GCTGGGCTGGA gtgg | 6/6 symmetric loop | |
| | | | AATTTTTGGAG | | |
| | | | CAGGTTTTCTGACTTCGGTCGG | | |
| | | | AAAACCCCT | | |
| FIG. 18 | 104 | 0.98.68 | CTTGAATGTGGTTGTTTTGCCG | 6/6 symmetric loop | 41.4 |
| | | (−15, +33) | GCACCATAGTGAGCCAGGAGG | 1 G/G mismatch | |
| | | | CCAAAGCACTCTCCAGTGAGA | 1 A/C mismatch | |
| | | | ACTCGGACACACAGCCTGAGT | 3/3 symmetric bulge | |
| | | | GAGCTGGGCTGGA gtgg | | |
| | | | AATTTTTGGAG | | |
| | | | CAGGTTTTCTGACTTCGGTCGG | 6/6 symmetric loop | |
| | | | AAAACCCCT | | |
| FIG. 21 | 105 | ABCA4 | This is the same sequence of the the | | |
| | | Macro- | guide 0.100.70 (−9, +33) inserted | | |
| | | footprint | above | | |
| FIG. 21 | 106 | GAPDH | CCACAGTTTCCCGGAGGGGCC | 1 A/C mismatch | 4.5 |
| FIG. 22 | | 100.70 A- | ATCCACAGTCTTCTGGGTGGC | | |
| | | C | AGTGATGGCATGGACTGTGGT | | |
| | | | CATGAGCCCTTCCACGATACC | | |
| | | | AAAGTTGTCATGGATG gtgg | | |
| | | | AATTTTTGGAG | | |
| | | | CAGGTTTTCTGACTTCGGTCGG | | |
| | | | AAAACCCCT | | |
| FIG. 21 | 107 | GAPDH | CCACAGTTTCCCGGAGGGGCC | 2/2 symmetric bulge | 6.3 |
| FIG. 22 | | Shaker | ATCCACAGTCTTCTGGGTGGC | 1 G/G mismatch | |
| | | mimicry | AGTGATGGCATGGACTGTGCA | 1 A/C mismatch | |
| | | | GATGAGCGCTTCCACGATAGG | 3/3 symmetric bulge | |
| | | | AAAGTTGTCATGGATG gtgg | | |
| | | | AATTTTTGGAG | | |
| | | | CAGGTTTTCTGACTTCGGTCGG | | |
| | | | AAAACCCCT | | |
| FIG. 21 | 108 | GAPDH | CCACAGTTTCCCGGAGGGGCC | 6/6 symmetric loop | 27 |
| FIG. 22 | | Shaker | ATCCACAGTCAAGACCGTGGC | 1 G/G mismatch | |
| | | mimicry | AGTGATGGCATGGACTGTGCA | 1 A/C mismatch | |
| | | −9, +33 | GATGAGCGCTTCCACCTATGG | 3/3 symmetric bulge | |
| | | | AAAGTTGTCATGGATG gtgg | 6/6 symmetric loop | |
| | | | AATTTTTGGAG | | |
| | | | CAGGTTTTCTGACTTCGGTCGG | | |
| | | | AAAACCCCT | | |
| FIG. 24 | 109 | Rab7a | TTCCCCCTGGAGAGATGAAAA | 1 A/C mismatch | 6.5 |
| FIG. 25 | | GAC | GCTTTTTGGCTCTTAAGTCTTT | | |
| FIG. 26 | | 100.70 A- | GATAAAAGGCGTACATAATTC | | |
| | | C | TTGTGCCTACTGTACAGAATA | | |
| | | | CTGCCGCTAGCTGGA gtgg | | |
| | | | AATTTTTGGAG | | |
| | | | CAGGTTTTCTGACTTCGGTCGG | | |
| | | | AAAACCCCT | | |
| FIG. 24 | 110 | Rab7a | TTCCCCCTGGAGAGATGAAAA | 2/2 symmetric bulge | 12 |
| FIG. 25 | | GAC | GCTTTTTGGCTCTTAAGTCTTT | 1 G/G mismatch | |
| FIG. 26 | | 100.70 | GATAAAAGGCGTACATAAAAG | 1 A/C mismatch | |
| | | Shaker | TTGTGCGTACTGTACAGACCA | 3/3 symmetric bulge | |
| | | mimicry | CTGCCGCTAGCTGGA gtgg | | |
| | | | AATTTTTGGAG | | |
| | | | CAGGTTTTCTGACTTCGGTCGG | | |
| | | | AAAACCCCT | | |
| FIG. 24 | 111 | Rab7a | TTCCCCCTGGAGAGATGAAAA | 6/6 symmetric loop | 28.5 |
| FIG. 25 | | GAC | GCTTTTTGGCAGCCTTGTCTTT | 1 G/G mismatch | |
| FIG. 26 | | 100.70 | GATAAAAGGCGTACATAAAAG | 1 A/C mismatch | |
| | | Shaker | TTGTGCGTACTGTAGTCTCCAC | 3/3 symmetric bulge | |
| | | mimicry | TGCCGCTAGCTGGA gtgg | 6/6 symmetric loop | |
| | | −9, +33 | AATTTTTGGAG | | |
| | | | CAGGTTTTCTGACTTCGGTCGG | | |
| | | | AAAACCCCT | | |
| FIG. 27 | 112 | APP GAA | CTGCAAAGAACACCAATTTTT | 1 A/C mismatch | 1.25 |
| FIG. 28 | | 100.70 A- | GATGATGAACTTCATATCCTG | | |
| FIG. 29 | | C | AGTCATGTCGGAATTCTGCAT | | |
| | | | CCATCTCCACTTCAGAGATCTC | | |
| | | | CTCCGTCTTGATATT gtgg | | |
| | | | AATTTTTGGAG | | |
| | | | CAGGTTTTCTGACTTCGGTCGG | | |
| | | | AAAACCCCT | | |
| FIG. 27 | 113 | APP GAA | CTGCAAAGAACACCAATTTTT | 2/2 symmetric bulge | 1.25 |
| FIG. 28 | | 100.70 | GATGATGAACTTCATATCCTG | 1 G/G mismatch | |
| FIG. 29 | | Shaker | AGTCATGTCGGAATTCTGCCA | 1 A/C mismatch | |
| | | mimicry | GCATCTCGACTTCAGAGATGA | 3/3 symmetric bulge | |
| | | | CCTCCGTCTTGATATT gtgg | | |
| | | | AATTTTTGGAG | | |
| | | | CAGGTTTTCTGACTTCGGTCGG | | |
| | | | AAAACCCCT | | |
| FIG. 27 | 114 | APP GAA | CTGCAAAGAACACCAATTTTT | 6/6 symmetric loop | 13.8 |
| FIG. 28 | | 100.70 | GATGATGAACAAGTTTTCCTG | 1 G/G mismatch | |
| FIG. 29 | | Shaker | AGTCATGTCGGAATTCTGCCA | 1 A/C mismatch | |
| | | mimicry | GCATCTCGACTTCAGTCTAGA | 3/3 symmetric bulge | |
| | | −9, +33 | CCTCCGTCTTGATATT gtgg | 6/6 symmetric loop | |
| | | | AATTTTTGGAG | | |
| | | | CAGGTTTTCTGACTTCGGTCGG | | |
| | | | AAAACCCCT | | |
| FIG. 30 | 115 | MAPT | TCCTGTCCCCCAACCCGTACGT | 1 A/C mismatch | 6.3 |
| FIG. 31 | | 100.70 A- | CCCAGCGTGATCTTCCATCACT | | |
| FIG. 32 | | C | TCGAACTCCTGGCGGGGCTCA | | |
| | | | GCCACCCTGGTTCAAAGTTCA | | |
| | | | CCTGATAGTCGACA gtgg | | |
| | | | AATTTTTGGAG | | |
| | | | CAGGTTTTCTGACTTCGGTCGG | | |
| | | | AAAACCCCT | | |
| FIG. 30 | 116 | MAPT | TCCTGTCCCCCAACCCGTACGT | 2/2 symmetric bulge | 5.3 |
| FIG. 31 | | 100.70 | CCCAGCGTGATCTTCCATCACT | 1 G/G mismatch | |
| FIG. 32 | | Shaker | TCGAACTCCTGGCGGGGGAGA | 1 A/C mismatch | |
| | | mimicry | GCCACGCTGGTTCAAAGAACA | 3/3 symmetric bulge | |
| | | | CCTGATAGTCGACA gtgg | | |
| | | | AATTTTTGGAG | | |
| | | | CAGGTTTTCTGACTTCGGTCGG | | |
| | | | AAAACCCCT | | |
| FIG. 30 | 117 | MAPT | TCCTGTCCCCCAACCCGTACGT | 6/6 symmetric loop | 43.5 |
| FIG. 31 | | 100.70 | CCCAGCGTGTAGAAGCATCAC | 1 G/G mismatch | |
| FIG. 32 | | Shaker | TTCGAACTCCTGGCGGGGGAG | 1 A/C mismatch | |
| | | mimicry | AGCCACGCTGGTTCTTTCAAC | 3/3 symmetric bulge | |
| | | −9, +33 | ACCTGATAGTCGACA gtgg | 6/6 symmetric loop | |
| | | | AATTTTTGGAG | | |
| | | | CAGGTTTTCTGACTTCGGTCGG | | |
| | | | AAAACCCCT | | |

While preferred embodiments have been shown and described herein, such embodiments are provided by way of example only. Numerous variations, changes, and substitutions are contemplated without departing from the present disclosure. It should be understood that various alternatives to the embodiments described herein can be employed.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

LENGTHY TABLES
The patent application contains a lengthy table section. A copy of the table is available in electronic form from the USPTO web site (https://seqdata.uspto.gov/?pageRequest=docDetail&DocID=US20250009904A1). An electronic copy of the table will also be available from the USPTO upon request and payment of the fee set forth in 37 CFR 1.19(b)(3).

1-126. (canceled)

127. An engineered guide RNA or a polynucleotide encoding the engineered guide RNA, wherein the engineered guide RNA comprises a sequence with complementarity to a sequence of a target RNA, wherein the complementarity is sufficient for the engineered guide RNA to hybridize to the target RNA, thereby forming a guide-target RNA scaffold that is a substrate for an adenosine deaminase acting on RNA (ADAR) enzyme, wherein the guide-target RNA scaffold comprises a micro-footprint and a barbell macro-footprint that each independently comprise a structural feature formed upon hybridization of the sequence of the engineered guide RNA to the sequence of the target RNA; wherein:
(a) the micro-footprint comprises a structural feature comprising an A/C mismatch formed between a target adenosine in the sequence of the target RNA and a cytosine of the engineered guide RNA; and
(b) the barbell macro-footprint comprises structural features:
(i) a first internal loop that is 5′ of the micro-footprint; and
(ii) a second internal loop that is 3′ of the micro-footprint; and wherein the guide-target RNA scaffold that comprises the micro-footprint and the barbell macro-footprint, upon contact with the ADAR enzyme, facilitates increased editing of the adenosine of the A/C mismatch in the sequence of the target RNA by the ADAR enzyme, relative to an otherwise comparable guide-target RNA scaffold lacking the barbell macro-footprint.

128. The engineered guide RNA or the polynucleotide encoding the engineered guide RNA of claim 127, wherein the micro-footprint comprises at least one additional structural feature selected from the group consisting of: a bulge, an internal loop, a mismatch, a wobble base pair, a hairpin, and any combination thereof.

129. The engineered guide RNA or the polynucleotide encoding the engineered guide RNA of claim 127, wherein the first internal loop or the second internal loop of the barbell macro-footprint is a symmetric internal loop.

130. The engineered guide RNA or the polynucleotide encoding the engineered guide RNA of claim 129, wherein the first internal loop and the second internal loop of the barbell macro-footprint are each symmetric internal loops that independently are 5/5, 6/6, 7/7, 8/8, 9/9, 10/10, 11/11, 12/12, 13/13, 14/14, 15/15, 16/16, 17/17, 18/18, 19/19, or 20/20 symmetric internal loops, wherein the first number is the number of nucleotides contributed to the symmetric internal loop from the engineered guide RNA side of the guide-target RNA scaffold and the second number is the number of nucleotides contributed to the symmetric internal loop from the target RNA side of the guide-target RNA scaffold.

131. The engineered guide RNA or the polynucleotide encoding the engineered guide RNA of claim 127, wherein the first internal loop or the second internal loop of the barbell macro-footprint is an asymmetric internal loop.

132. The engineered guide RNA or the polynucleotide encoding the engineered guide RNA of claim 127, wherein the guide-target RNA scaffold that comprises the micro-footprint and the barbell macro-footprint, upon contact with the ADAR enzyme, facilitates decreased editing of an off-target adenosine in the sequence of the target RNA by the ADAR enzyme, relative to an otherwise comparable guide-target RNA scaffold lacking the barbell macro-footprint.

133. The engineered guide RNA or the polynucleotide encoding the engineered guide RNA of claim 132, wherein the first internal loop of the barbell macro-footprint is positioned from about 1 base away from the A/C mismatch to about 30 bases away from the A/C mismatch with respect to a base of the first internal loop that is most proximal to the A/C mismatch.

134. The engineered guide RNA or the polynucleotide encoding the engineered guide RNA of claim 133, wherein the first internal loop of the barbell macro-footprint is positioned 4 bases, 5 bases, 6 bases, 8 bases, 10 bases, 11 bases, 15 bases, 18 bases, or 22 bases away from the A/C mismatch with respect to the base of the first internal loop that is most proximal to the A/C mismatch.

135. The engineered guide RNA or the polynucleotide encoding the engineered guide RNA of claim 127, wherein the second internal loop of the barbell macro-footprint is positioned from about 12 bases away from the A/C mismatch to about 40 bases away from the A/C mismatch with respect to a base of the second internal loop that is most proximal to the A/C mismatch.

136. The engineered guide RNA or the polynucleotide encoding the engineered guide RNA of claim 135, wherein the second internal loop of the barbell macro-footprint is positioned 26 bases, 27 bases, 28 bases, 30 bases, 32 bases, 33 bases, 35 bases, 36 bases, or about 40 bases away from the A/C mismatch with respect to the base of the second internal loop that is most proximal to the A/C mismatch.

137. An engineered guide RNA or a polynucleotide encoding the engineered guide RNA, wherein the engineered guide RNA comprises a sequence with complementarity to a sequence of a target RNA, wherein the complementarity is sufficient for the engineered guide RNA to hybridize to the target RNA, thereby forming a guide-target RNA scaffold that is a substrate for an adenosine deaminase acting on RNA (ADAR) enzyme, wherein the guide-target RNA scaffold comprises a micro-footprint and a barbell macro-footprint; wherein:
(a) the micro-footprint comprises a structural feature formed upon hybridization of the sequence of the engineered guide RNA to the sequence of the target RNA that is selected from the group consisting of: a bulge, an internal loop, a mismatch, a wobble base pair, a hairpin, and any combination thereof, and
(b) the barbell macro-footprint comprises a first internal loop and a second internal loop that are each formed upon hybridization of the sequence of the engineered guide RNA to the sequence of the target RNA, wherein the first internal loop is 5′ of the micro-footprint and the second internal loop is 3′ of the micro-footprint;
wherein the guide-target RNA scaffold that comprises the micro-footprint and the barbell macro-footprint, upon contact with the ADAR enzyme, facilitates increased editing of a target adenosine in the sequence of the target RNA by the ADAR enzyme, relative to an otherwise comparable guide-target RNA scaffold lacking the barbell macro-footprint; and wherein:
(i) the engineered guide RNA is a linear guide RNA;
(ii) the first internal loop or the second internal loop is a 5/5, 6/6, or 7/7 symmetric internal loop;
(iii) at least one of the first internal loop or the second internal loop is not an 8/8 symmetric internal loop; or
(iv) any combination of (i), (ii), and/or (iii).

138. An engineered guide RNA or a polynucleotide encoding the engineered guide RNA, wherein the engineered guide RNA comprises a sequence with complementarity to a sequence of a target RNA, wherein the complementarity is sufficient for the engineered guide RNA to hybridize to the target RNA, thereby forming a guide-target RNA scaffold that is a substrate for an adenosine deaminase acting on RNA (ADAR) enzyme, wherein the guide-target RNA scaffold comprises a micro-footprint and a barbell macro-footprint; wherein:
(a) the micro-footprint comprises a structural feature formed upon hybridization of the sequence of the engineered guide RNA to the sequence of the target RNA that is selected from the group consisting of: a bulge, an internal loop, a mismatch, a wobble base pair, a hairpin, and any combination thereof, and
(b) the barbell macro-footprint comprises a first internal loop and a second internal loop that are each formed upon hybridization of the sequence of the engineered guide RNA to the sequence of the target RNA, wherein the first internal loop is 5′ of the micro-footprint and the second internal loop is 3′ of the micro-footprint;
wherein the target RNA is selected from the group consisting of: ABCA4, APP, CFTR, DMPK, DUX4, GBA, GRN, HEXA, LIPA, LRRK2, MAPT, PINK1, PMP22, SERPINA1, SNCA, SOD1, a fragment of any one of these, and any combination thereof.

139. The engineered guide RNA or the polynucleotide encoding the engineered guide RNA of claim 138, wherein the structural feature of the micro-footprint is the mismatch, and wherein the mismatch is an A/C mismatch formed between a target adenosine in the sequence of the target RNA and a cytosine of the engineered guide RNA.

140. The engineered guide RNA or the polynucleotide encoding the engineered guide RNA of claim 139, wherein the target RNA is ABCA4.

141. The engineered guide RNA or the polynucleotide encoding the engineered guide RNA of claim 140, wherein the first internal loop of the barbell macro-footprint is positioned from about 5 bases away from the A/C mismatch to about 15 bases away from the A/C mismatch with respect to a base of the first internal loop that is most proximal to the A/C mismatch, and wherein the second internal loop of the barbell macro-footprint is positioned from about 12 bases away from the A/C mismatch to about 40 bases away from the A/C mismatch with respect to a base of the second internal loop that is most proximal to the A/C mismatch.

142. The engineered guide RNA or the polynucleotide encoding the engineered guide RNA of claim 140, wherein the engineered guide RNA comprises a polynucleotide sequence with at least 80%, 85%, 90%, 92%, 95%, 97%, or 99% sequence identity to any one of SEQ ID NO: 1-105, 2729-2761, or 2772-2843.

143. The engineered guide RNA or the polynucleotide encoding the engineered guide RNA of claim 139, wherein the target RNA is APP.

144. The engineered guide RNA or the polynucleotide encoding the engineered guide RNA of claim 143, wherein the first internal loop of the barbell macro-footprint is positioned from about 5 bases away from the A/C mismatch to about 20 bases away from the A/C mismatch with respect to the base of the first internal loop that is most proximal to the A/C mismatch; and wherein the second internal loop of the barbell macro-footprint is positioned from about 15 bases away from the A/C mismatch to about 40 bases away from the A/C mismatch with respect to the base of the second internal loop that is most proximal to the A/C mismatch.

145. The engineered guide RNA or the polynucleotide encoding the engineered guide RNA of claim 143, wherein the engineered guide RNA comprises a polynucleotide sequence with at least 80%, 85%, 90%, 92%, 95%, 97%, or 99% sequence identity to any one of SEQ ID NO: 112-114.

146. The engineered guide RNA or the polynucleotide encoding the engineered guide RNA of claim 139, wherein the target RNA is SERPINA1.

147. The engineered guide RNA or the polynucleotide encoding the engineered guide RNA of claim 146, wherein the first internal loop of the barbell macro-footprint is positioned from about 5 bases away from the A/C mismatch to about 20 bases away from the A/C mismatch with respect to the base of the first internal loop that is most proximal to the A/C mismatch, and wherein the second internal loop of the barbell macro-footprint is positioned from about 12 bases away from the A/C mismatch to about 40 bases away from the A/C mismatch with respect to the base of the second internal loop that is most proximal to the A/C mismatch.

148. The engineered guide RNA or the polynucleotide encoding the engineered guide RNA of claim 146, wherein the engineered guide RNA comprises a polynucleotide sequence with at least 80%, 85%, 90%, 92%, 95%, 97%, or 99% sequence identity to any one of SEQ ID NO: 2762-2768 or 3083-3086.

149. The engineered guide RNA or the polynucleotide encoding the engineered guide RNA of claim 139, wherein the target RNA is LRRK2.

150. The engineered guide RNA or the polynucleotide encoding the engineered guide RNA of claim 149, wherein the first internal loop of the barbell macro-footprint is positioned from about 7 bases away from the A/C mismatch to about 30 bases away from the A/C mismatch with respect to the base of the first internal loop that is most proximal to the A/C mismatch; and wherein the second internal loop of the barbell macro-footprint is positioned from about 18 bases away from the A/C mismatch to about 34 bases away from the A/C mismatch with respect to the base of the second internal loop that is most proximal to the A/C mismatch.

151. The engineered guide RNA or the polynucleotide encoding the engineered guide RNA of claim 149, wherein the engineered guide RNA comprises a polynucleotide sequence with at least 80%, 85%, 90%, 92%, 95%, 97%, or 99% sequence identity to any one of SEQ ID NO: 118-167, 2686-2728, 2769-2771, 2844-3078, or 3081-3082.

152. The engineered guide RNA or the polynucleotide encoding the engineered guide RNA of claim 139, wherein the target RNA is SNCA.

153. The engineered guide RNA or the polynucleotide encoding the engineered guide RNA of claim 152, wherein the first internal loop of the barbell macro-footprint is positioned from about 4 bases away from the A/C mismatch to about 18 bases away from the A/C mismatch with respect to the base of the first internal loop that is most proximal to the A/C mismatch; and wherein the second internal loop of the barbell macro-footprint is positioned from about 26 bases away from the A/C mismatch to about 33 bases away from the A/C mismatch with respect to the base of the second internal loop that is most proximal to the A/C mismatch.

154. The engineered guide RNA or the polynucleotide encoding the engineered guide RNA of claim 152, wherein the engineered guide RNA comprises a polynucleotide sequence with at least 80%, 85%, 90%, 92%, 95%, 97%, or 99% sequence identity to any one of SEQ ID NO: 2480-2681.

155. The engineered guide RNA or the polynucleotide encoding the engineered guide RNA of claim 139, wherein the target RNA is MAPT.

156. The engineered guide RNA or the polynucleotide encoding the engineered guide RNA of claim 155, wherein the first internal loop of the barbell macro-footprint is positioned from about 5 bases away from the A/C mismatch to about 18 bases away from the A/C mismatch with respect to the base of the first internal loop that is most proximal to the A/C mismatch; and wherein the second internal loop of the barbell macro-footprint is positioned from about 12 bases away from the A/C mismatch to about 36 bases away from the A/C mismatch with respect to the base of the second internal loop that is most proximal to the A/C mismatch.

157. The engineered guide RNA or the polynucleotide encoding the engineered guide RNA of claim 155, wherein the engineered guide RNA comprises a polynucleotide sequence with at least 80%, 85%, 90%, 92%, 95%, 97%, or 99% sequence identity to any one of SEQ ID NO: 115-117, 1519-2479 or 2682-2685.

158. The engineered guide RNA or the polynucleotide encoding the engineered guide RNA of claim 139, wherein the target RNA is DUX4.

159. The engineered guide RNA or the polynucleotide encoding the engineered guide RNA of claim 158, wherein the first internal loop of the barbell macro-footprint is positioned from about 1 base away from the A/C mismatch to about 22 bases away from the A/C mismatch with respect to the base of the first internal loop that is most proximal to the A/C mismatch; and wherein the second internal loop of the barbell macro-footprint is positioned from about 15 bases away from the A/C mismatch to about 40 bases away from the A/C mismatch with respect to the base of the second internal loop that is most proximal to the A/C mismatch.

160. The engineered guide RNA or the polynucleotide encoding the engineered guide RNA of claim 158, wherein the engineered guide RNA comprises a polynucleotide sequence with at least 80%, 85%, 90%, 92%, 95%, 97%, or 99% sequence identity to any one of SEQ ID NO: 172-1518.

161. A method of treating a disease in a subject in need thereof, the method comprising administering to the subject in need thereof an effective amount of an engineered guide RNA or a polynucleotide encoding the engineered guide RNA, wherein the engineered guide RNA comprises a sequence with complementarity to a sequence of a target RNA, wherein the complementarity is sufficient for the engineered guide RNA to hybridize to the target RNA, thereby forming a guide-target RNA scaffold that is a substrate for an adenosine deaminase acting on RNA (ADAR) enzyme, wherein the guide-target RNA scaffold comprises a micro-footprint and a barbell macro-footprint; wherein:
(a) the micro-footprint comprises a structural feature formed upon hybridization of the sequence of the engineered guide RNA to the sequence of the target RNA that is selected from the group consisting of: a bulge, an internal loop, a mismatch, a wobble base pair, a hairpin, and any combination thereof, and
(b) the barbell macro-footprint comprises a first internal loop and a second internal loop that are each formed upon hybridization of the sequence of the engineered guide RNA to the sequence of the target RNA, wherein the first internal loop is 5′ of the micro-footprint and the second internal loop is 3′ of the micro-footprint; and
wherein the disease is selected from the group consisting of: a macular degeneration, a Stargardt Disease, a Parkinson's disease, an Alzheimer's disease, a facioscapulohumeral muscular dystrophy (FSHD), a tauopathy, a dementia, a liver cirrhosis, an alpha-1 antitrypsin deficiency (AAT deficiency), and any combination thereof.
